SEQUENCE LISTING (71 sequences)

SEQ ID NO: 1 - protein, 219 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 2 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 3 - protein, 15 aa, synthetic construct
GGGGSGGGGSGGGGS

SEQ ID NO: 4 - protein, 287 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 5 - protein, 352 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 6 - protein, 620 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 638 aa, synthetic construct
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 370 aa, synthetic construct
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 9 - DNA, 657 bp, Homo sapiens
  source 1..657: mol_type = genomic DNA
agcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggag

SEQ ID NO: 10 - protein, 219 aa, Homo sapiens
SGTTNTVAAYNLTWKSTNFATALEWEPKPVNQVYTVQISTKSGDWKSKCFYTTDTECALTDEIVKDVKQTYLARVFSYPAGNVESTGSAGEPLYENSPEFTPYLETNLGQPTIQSFEQVGTKVNVTVEDERTLVARNNTALSLRDVFGKDLIYTLYYWKSSSSGKKTAKTNTNEFLIDVDKGENYCFSVQAVIPSRTVNRKSTDSPVECMGQEKGEFRE

SEQ ID NO: 11 - protein, 219 aa, Homo sapiens
SGTTNTVAAYNLTWKSTNFATALEWEPKPVNQVYTVQISTKSGDAKSKCFYTTDTECALTDEIVKDVKQTYLARVFSYPAGNVESTGSAGEPLAENSPEFTPYLETNLGQPTIQSFEQVGTKVNVTVEDERTLVARNNTALSLRDVFGKDLIYTLYYWKSSSSGKKTAKTNTNEFLIDVDKGENYCFSVQAVIPSRTVNRKSTDSPVECMGQEKGEFRE

SEQ ID NO: 12 - protein, 223 aa, Mus musculus
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGE

SEQ ID NO: 13 - protein, 224 aa, Rattus norvegicus
AGTPPGKAFNLTWISTDFKTILEWQPKPTNYTYTVQISDRSRNWKYKCTGTTDTECDLTDEIVKDVNWTYEARVLSVPWRNSTHGKETLFGTHGEEPPFTNARKFLPYRDTKIGQPVIQKYEQGGTKLKVTVKDSFTLVRKNGTFLTLRQVFGNDLGYILTYRKDSSTGRKTNTTHTNEFLIDVEKGVSYCFFAQAVIFSRKTNHKSPESITKCTEQWKSVLGE

SEQ ID NO: 14 - DNA, 45 bp, synthetic construct
  source 1..45: mol_type = other DNA
ggcggtggaggatccggaggaggtggctccggcggcggaggatct

SEQ ID NO: 15 - protein, 8 aa, synthetic construct
GGGSGGGS

SEQ ID NO: 16 - protein, 65 aa, synthetic construct
ITCPPPMSVEHADIWVKSYSLYSRERYICNSGFKRKAGTSSLTECVLNKATNVAHWTTPSLKCIR

SEQ ID NO: 17 - DNA, 195 bp, synthetic construct
  source 1..195: mol_type = other DNA
attacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 18 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 19 - DNA, 342 bp, Homo sapiens
  source 1..342: mol_type = genomic DNA
aactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 20 - protein, 18 aa, synthetic construct
MKWVTFISLLFLFSSAYS

SEQ ID NO: 21 - DNA, 54 bp, synthetic construct
  source 1..54: mol_type = other DNA
atgaaatgggtgacctttatttctttactgttcctctttagcagcgcctactcc

SEQ ID NO: 22 - DNA, 54 bp, synthetic construct
  source 1..54: mol_type = other DNA
atgaagtgggtcacatttatctctttactgttcctcttctccagcgcctacagc

SEQ ID NO: 23 - DNA, 54 bp, synthetic construct; identical to SEQ ID NO: 21
  source 1..54: mol_type = other DNA

SEQ ID NO: 24 - protein, 16 aa, synthetic construct
MKCLLYLAFLFLGVNC

SEQ ID NO: 25 - protein, 58 aa, synthetic construct
MGQIVTMFEALPHIIDEVINIVIIVLIIITSIKAVYNFATCGILALVSFLFLAGRSCG

SEQ ID NO: 26 - protein, 97 aa, synthetic construct
MPNHQSGSPTGSSDLLLSGKKQRPHLALRRKRRREMRKINRKVRRMNLAPIKEKTAWQHLQALISEAEEVLKTSQTPQNSLTLFLALLSVLGPPVTG

SEQ ID NO: 27 - protein, 30 aa, synthetic construct
MDSKGSSQKGSRLLLLLVVSNLLLCQGVVS

SEQ ID NO: 28 - protein, 16 aa, synthetic construct
MKCLLYLAFLFLGVNC

SEQ ID NO: 29 - protein, 15 aa, synthetic construct
GLNDIFEAQKIEWHE

SEQ ID NO: 30 - protein, 26 aa, synthetic construct
KRRWKKNFIAVSAANRFKKISSSGAL

SEQ ID NO: 31 - protein, 6 aa, synthetic construct
EEEEEE

SEQ ID NO: 32 - protein, 13 aa, synthetic construct
GAPVPYPDPLEPR

SEQ ID NO: 33 - protein, 8 aa, synthetic construct
DYKDDDDK

SEQ ID NO: 34 - protein, 9 aa, synthetic construct
YPYDVPDYA

SEQ ID NO: 35 - protein, 5 aa, synthetic construct
HHHHH

SEQ ID NO: 36 - protein, 6 aa, synthetic construct
HHHHHH

SEQ ID NO: 37 - protein, 7 aa, synthetic construct
HHHHHHH

SEQ ID NO: 38 - protein, 8 aa, synthetic construct
HHHHHHHH

SEQ ID NO: 39 - protein, 9 aa, synthetic construct
HHHHHHHHH

SEQ ID NO: 40 - protein, 10 aa, synthetic construct
HHHHHHHHHH

SEQ ID NO: 41 - protein, 10 aa, synthetic construct
EQKLISEEDL

SEQ ID NO: 42 - protein, 18 aa, synthetic construct
TKENPRSNQEESYDDNES

SEQ ID NO: 43 - protein, 15 aa, synthetic construct
KETAAAKFERQHMDS

SEQ ID NO: 44 - protein, 38 aa, synthetic construct
MDEKTTGWRGGHVVEGLAGELEQLRARLEHHPQGQREP

SEQ ID NO: 45 - protein, 13 aa, synthetic construct
SLAELLNAGLGGS

SEQ ID NO: 46 - protein, 8 aa, synthetic construct
TQDPSRVG

SEQ ID NO: 47 - protein, 12 aa, synthetic construct
PDRVRAVSHWSS

SEQ ID NO: 48 - protein, 8 aa, synthetic construct
WSHPQFEK

SEQ ID NO: 49 - protein, 6 aa, synthetic construct
CCPGCC

SEQ ID NO: 50 - protein, 10 aa, synthetic construct
EVHTNQDPLD

SEQ ID NO: 51 - protein, 14 aa, synthetic construct
GKPIPNPLLGLDST

SEQ ID NO: 52 - protein, 11 aa, synthetic construct
YTDIEMNRLGK

SEQ ID NO: 53 - protein, 8 aa, synthetic construct
DLYDDDDK

SEQ ID NO: 54 - protein, 10 aa, synthetic construct
EQKLISEEDL

SEQ ID NO: 55 - DNA, 408 bp, Homo sapiens
  source 1..408: mol_type = genomic DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgat

SEQ ID NO: 56 - DNA, 408 bp, Homo sapiens
  source 1..408: mol_type = genomic DNA
attcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 57 - DNA, 861 bp, synthetic construct
  source 1..861: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 58 - DNA, 1860 bp, synthetic construct
  source 1..1860: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 59 - DNA, 1914 bp, synthetic construct
  source 1..1914: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 60 - DNA, 1056 bp, synthetic construct
  source 1..1056: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 61 - DNA, 1110 bp, synthetic construct
  source 1..1110: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 62 - protein, 352 aa, synthetic construct; identical to SEQ ID NO: 5

SEQ ID NO: 63 - DNA, 1056 bp, synthetic construct; identical to SEQ ID NO: 60
  source 1..1056: mol_type = other DNA

SEQ ID NO: 64 - protein, 370 aa, synthetic construct; identical to SEQ ID NO: 8

SEQ ID NO: 65 - DNA, 1110 bp, synthetic construct; identical to SEQ ID NO: 61
  source 1..1110: mol_type = other DNA

SEQ ID NO: 66 - protein, 620 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 67 - DNA, 1860 bp, synthetic construct
  source 1..1860: mol_type = other DNA
atcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgactcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaaactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 68 - protein, 637 aa, synthetic construct
MGVKVLFALICIAVAEAIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - DNA, 1911 bp, synthetic construct
  source 1..1911: mol_type = other DNA
atgggagtgaaagttctttttgcccttatttgtattgctgtggccgaggccatcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgactcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaaactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 70 - protein, 114 aa, synthetic construct
NWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 71 - DNA, 342 bp, synthetic construct
  source 1..342: mol_type = other DNA
aactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

(19) United States
(12) Patent Application Publication
(10) Pub. No.: US 2023/0125173 A1
(43) Pub. Date: Apr. 27, 2023

(54) MULTI-CHAIN CHIMERIC POLYPEPTIDES AND USE THEREOF IN THE TREATMENT OF LIVER DISEASES

(71) Applicant: HCW Biologics, Inc., Miramar, FL (US)

(72) Inventors: Hing C. Wong, Miramar, FL (US); Xiaoyun Zhu, Miramar, FL (US); Pallavi Chaturvedi, Miramar, FL (US); Varghese George, Miramar, FL (US); Niraj Shrestha, Miramar, FL (US); Michael Dee, Miramar, FL (US)

(73) Assignee: HCW Biologics, Inc., Miramar, FL (US)

(21) Appl. No.: 17/819,240

(22) Filed: Aug. 11, 2022

Related U.S. Application Data
(60) Provisional application No. 63/330,757, filed on Apr. 13, 2022.
(60) Provisional application No. 63/232,140, filed on Aug. 11, 2021.

(51) Int. Cl. A61K 47/68; A61K 47/64; A61K 47/65; A61P 1/16

(52) U.S. Cl. CPC A61K 47/6813; A61K 47/641; A61K 47/65; A61P 1/16

(57) ABSTRACT

Provided herein are multi-chain chimeric polypeptides and use thereof in the treatment of liver diseases.

### CROSS-REFERENCE TO RELATED APPLICATION

This application claims priority to U.S. Provisional Application Ser. No. 63/232,140, filed on Aug. 11, 2021, and U.S. Provisional Application Ser. No. 63/330,757, filed on Apr. 13, 2022, each of which are incorporated herein by reference in their entirety.

### SEQUENCE LISTING

This application contains a Sequence Listing that has been submitted electronically as an XML, file named 47039-0025001_SL_ST26.XML. The XML file, created on Aug. 9, 2022, is 83,507 bytes in size. The material in the XML file is hereby incorporated by reference in its entirety.

### TECHNICAL FIELD

The present disclosure relates to the field of biotechnology, and more specifically, to antigen-binding molecules and the treatment of liver diseases.

### BACKGROUND

Tissue factor (TF), a 263 amino acid integral membrane glycoprotein with a molecular weight of ˜46 kDa and the trigger protein of the extrinsic blood coagulation pathway, is the primary initiator of coagulation in vivo. Tissue factor, normally not in contact with circulating blood, initiates the coagulation cascade upon exposure to the circulating coagulation serine protease factors. Vascular damage exposes sub-endothelial cells expressing tissue factor, resulting in the formation of a calcium-dependent, high-affinity complex with pre-existing plasma factor VIIa (FVIIa). Binding of the serine protease FVIIa to tissue factor promotes rapid cleavage of FX to FXa and FIX to FIXa. The proteolytic activity of the resulting FXa and an active membrane surface then inefficiently converts a small amount of prothrombin to thrombin. The thrombin generated by FXa initiates platelet activation and activates minute amounts of the pro-cofactors factor V (FV) and factor VIII (FVIII) to become active cofactors, factor Va (FVa) and factor VIIIa (FVIIIa). FIXa complexes with FVIIIa on the platelet surface forming the intrinsic tenase complex, which results in rapid generation of FXa. FXa complexes with FVa to form the pro-thrombinase complex on the activated platelet surface which results in rapid cleavage of prothrombin to thrombin.

In addition to the tissue factor-FVIIa complex, a recent study showed that the tissue factor-FVIIa-FXa complex can activate FVIII, which would provide additional levels of FVIIIa during the initiation phase. The extrinsic pathway is paramount in initiating coagulation via the activation of limited amounts of thrombin, whereas the intrinsic pathway maintains coagulation by dramatic amplification of the initial signal.

Much of the tissue factor expressed on a cell surface is “encrypted,” which must be “decrypted” for full participation in coagulation. The mechanism of “decryption” of cell-surface tissue factor is still unclear at this time, however, exposure of anionic phospholipids plays a major role in this process. Healthy cells actively sequester anionic phospholipids such as phosphatidyl serine (PS) to the inner leaflet of the plasma membrane. Following cellular damage, activation, or increased levels of cytosolic Ca', this bilayer asymmetry is lost, resulting in increased PS exposure on the outer leaflet, which increases the specific activity of cell-surface tissue factor-FVIIa complexes. PS exposure is known to decrease the apparent Km for activation of FIX and FX by tissue factor-FVIIa complexes, but additional mechanisms could include conformational rearrangement of tissue factor or tissue factor-FVIIa and subsequent exposure of substrate binding sites.

### SUMMARY

Provided herein are methods of treating a liver disease or a metabolic syndrome in a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. In some embodiments of any of the methods described herein, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments of any of the methods described herein, the metabolic syndrome is selected from the group of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

Also provided herein are methods of reducing one or more of the rate of: progression from non-alcoholic fatty liver disease (NAFL) to non-alcoholic steatohepatitis (NASH), progression from NASH to cirrhosis, and progression from cirrhosis to hepatocellular carcinoma, that include administering to a subject identified or diagnosed as having NAFL, NASH, or cirrhosis, a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. In some embodiments of any of the methods described herein, the method results in a decreasing in the rate of progression from NAFL to NASH. In some embodiments of any of the methods described herein, the method results in decreasing the rate of progression of NASH to cirrhosis. In some embodiments of any of the methods described herein, the method results in decreasing the rate of progression from cirrhosis to hepatocellular carcinoma.

Also provided herein are methods of reducing inflammation in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of decreasing gluconeogenesis in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of decreasing lipogenesis in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βPRII.

Also provided herein are methods of decreasing hepatocytic senescence in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of rebalancing metabolic function in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII.

Also provided herein are methods of modulating expression of one or more genes in Tables 1-4 in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGT-βRII) and the second target-binding domain binds specifically to a ligand of TGF-βRII. In some embodiments of any of the methods described herein, the administering results in a decrease in the expression of one or more genes in the liver of the subject selected from the group consisting of: ACSS1, RETN, SLC2A4, PDK4, PNPLA3, GADD45B, PPARGC1A, CAV1, ENDOD1, REG3G, IGHG3, IGHG2B, SCGB3A1, GLYCAM1, IGHG2C, IGKC, LTF, MS4A1, JCHAIN, CD19, IGHM, IFI27L2A, ACKR3, LSP1, PMEPA1, CORO1A, GPX3, MYH8, NPPA, TCAP, FLNC, SLC36A2, MYH6, ACTC1, ACTA2, and TPM2, as compared to the level of expression of the one or more genes in the subject prior to the administering. In some embodiments of any of the methods described herein, the administering results in an increase in the expression of one or more genes in the liver of the subject selected from the group consisting of: SLC34A2, and CISH, as compared to the level of expression of the one or more genes in the subject prior to the administering. In some embodiments of any of the methods described herein, the administering results in a decrease in the expression of one or more genes in the liver of the subject selected from the group consisting of: CSF3R, IFI27L2A, GM17066, GNL3, FABP1, GM14303, AURKA, RPL14-PS1, QTRT2, G6PC, C8B, DYNLL1, LCN2, LRG1, CEBPD, COL4A3, ST3GAL5, RSAD2, 9330162G02RIK, PINX1, SRA1, SPATA2L, PNRC1, MUP20, IL6RA, APOA1, IL1B, WDR54, CTCFLOS, GM16973, 4632427E13RIK, IGHG2B, TGFB1I1, SELENBP2, SEMA6B, NEXN, ZFP653, NOB1, PCK1, FAM25C, MAPK15, GM16551, ESM1, RPL37RT, FAM133B, PDE8B, TUT1, S100A11, PDILT, PPARD, IER2, GM15401, MX2, WNK4, GOS2, BC005561, AA986860, JDP2, GM26982, NOP58, ACTB, GM14586, RPP38, GM13436, NT5DC2, IMPDH1, CYTIP, AI846148, CHKA, GM37963, NROB2, CYP4A32, ALKBH2, FAU-PS2, PPP1R15A, KLF2, SLC25A22, GM13341, IGHM, SATB1, SNRPF, DNASE1L2, CD3EAP, GM2788, DANCR, ZFP612, NOP56, JUND, ID1, HSPB1, KLHDC8A, KLF10, ANGPT2, THBS1, GM44891, GM9752, ABLIM3, PTGES, GM28438, 2410002F23RIK, FOSL2, CRIP3, JUN, ALAS1, GM2000, RHOC, LMCD1, GM2061, GM42595, GM11478, IKZF2, PNLDC1, COMTD1, SNORA31, COL20A1, AKAP12, C1QTNF12, 1810032008RIK, 2310033P09RIK, GM47528, SERPINE2, NPFF, SERPINA3K, RFXANK, IGKV5-39, NAB2, MAFF, CEP85, CSAD, LTB4R1, 1810012K08RIK, BCL7C, NRBP2, NLE1, ALKBH1, ARIDSA, CFAP43, GM45767, CD8A, PPRC1, GM26870, TMC7, BCL6B, GM16348, GM26981, SLC16A3, TNFRSF12A, CYP2J9, NR4A2, MMP9, MIR17HG, TMEM191C, PCDH11X, HILPDA, RAPGEF4, GM17300, SLC25A47, KCNJ2, NYAP1, LAX1, RPS19-PS3, HES1, RGS16, DUSP1, GM43323, ASB4, MUC6, GM15502, UNG, FOXQ1, GM17936, UBE2C, SLC16A6, MIR7052, NLRP12, GM14286, FGF21, KLFS, GM37969, PF4, GM21738, HOTAIRM1, GM6493, LOR, MFSD2B, MATK, SYNE4, GM44694, TRBC1, GM37274, PLN, CXCR4, PHF24, SNORD104, SERPINA7, RGS4, TCIM, EGFR, GM37760, FBXL22, TEDC2, ENHO, GM26917, GM43775, 4833411C07RIK, GM45053, INHBB, OPN3, SNHG15, B230206H07RIK, KCNE3, GM43305, C530043K16RIK, KLF4, LEPR, JCHAIN, TSKU, LGALS4, PCP4L1, GM44829, DUSP8, GM44620, IGFBP1, JUNB, GM32017, GM2814, GM37144, MYADML2OS, GM37666, HDC, SLFN4, A530041M06RIK, GM43359, GM2602, GM10277, FAM222A, FOXA3, AOC2, SERPINA1E, CTXN1, RAPGEF4OS2, SOCS2, PPAN, PRKAG2OS1, GADD45B, HOXAS, GRHL1, EIF4EBP3, OSGIN1, GM28513, MAP3K6, SLC34A2, B630019A1ORIK, IGKC, PLIN4, ANGPTL4, DUSPS, EGR1, GM42507, GM14257, APOLD1, IER3, ZBTB16, GM37033, IGLC1, GADD45G, IGLC3, GM45244, RGS1, CXCL1, RNF225, GM44005, ANKRD37, NR4A1, GM8893, GM26762, CDKN1A, 5330406M23RIK, IGLV1, IGKV3-2, FOS, GM43637, IGKV3-10, S100A9, GM15622, S100A8, MT1, RETNLG, MT2, IGKV19-93, GM45774, and SERPINA4-PS1, as compared to the level of expression of the one or more genes in the subject prior to the administering. In some embodiments of any of the methods described herein, the administering results in an increase in the expression of one or more genes in the liver of the subject selected from the group consisting of: DBP, IGKV4-55, PER3, MUP-PS10, GPAM, TMPRSS4, MUP-PS14, AC166078.1, MUP-PS12, GM2065, A530020G20RIK, ACSS2OS, DCLK3, KLF12, GM44669, MFSD9, B4GALNT3, GM3776, TMEM167-PS1, KRT23, LMBRD2, GM22935, SULT2A-PS1, SNAI3, GM15908, MIR6392, ACSS2, NR1D1, BC049987, CCDC85C, CES2C, ACPP, MUP2, PTK6, UGT1A5, 1810008I18RIK, IL22RA1, ACSS3, ADNP, RDH16, SNTB1, 4933411K16RIK, NTRK2, EXTL1, PSTPIP2, RASSF6, AQP4, UGT1A9, PROM1, ZFP608, FAM13A, NFE2, TEF, TNFAIP8L3, SCD1, MMD2, SYNE3, ACLY, C330021F23RIK, STON2, LRFN4, HHIPL1, WNT9B, NR1D2, 1810049J17RIK, PDPR, NA, GM45884, SLC2A5, FAM83F, ZFP526, SGK2, GM43080, DEAF1, MEI, BMF, WDFY2, ADCY9, CLSTN3, ACOT11, LYST, LRTM1, OAT, VPS13C, E330011021RIK, P2RY4, GM11437, RWDD2A, SVIL, ECHDC1, TRIM14, SLC10A5, TRHDE, MASP1, 2900097C17RIK, NDST1, RDH9, 1110002L01RIK, ABTB2, RGR, ACACB, SACM1L, DYRK2, ROBO1, GM44744, EIF4EBP2, KLHL24, CYP2A5, TIAM2, RAB43, GM13855, 9130409I23RIK, STON1, USP9X, UGT3A1, 9030616G12RIK, DOCK8, KLB, ACE, VLDLR, PCDHGC3, ABCA6, 4932422M17RIK, GM45838, FARP2, GM47205, SP4, UGT1A6B, KLHL28, D130043K22RIK, ASIC5, PM20D2, A1CF, SORBS1, SLC10A2, GM10642, UTP14B, GM38394, AFP, INSIG1, HNF1AOS2, METTL4, LSS, MTMR9, HMGCR, GDAP10, ADRA1A, ZFP773, CRKL, CHRNE, STARD13, CRY2, FADS2, COG5, FV1, RCAN2, ABCB1A, PPARA, ATP7A, MVD, 2610037D02RIK, TNFRSF14, SUCNR1, ECI3, ABCC4, LNCBATE1, MINDY2, BTBD7, 4933404O12RIK, ABCD1, FMN1, FNIP2, ABHD15, NKX2-6, C77080, GM43611, SGTB, ACSL3, NR5A2, FAM198A, KCTD7, ACACA, ZFP955B, SULT2A3, FZD4, FASN, CYP3A59, ZFP354B, TNFSF10, SESN3, MN1, RNF152, DHCR24, SPHK2, SYTLS, GM6652, BAHCC1, GAREM1, MFSD4A, HGF, GM3571, NOS1AP, DIXDC1, KANK1, REPS2, ASAH2, SEMA3B, RNF103, ZC3H12C, CDS2, DCUN1D4, 2900026A02RIK, CYYR1, EEPD1, P2RY2, CYP2C39, SEC22C, EHHADH, ABCA3, HIPK2, RBM20, GRAMD4, FCHSD2, MOB3A, HMGN3, KLHDC7A, VCP-RS, TERT, CYP3A41B, ARL13B, ZC3H12D, TLCD2, SNHG11, SORL1, GPR157, DNAJA4, TMEM253, TACO1, SPATA5L1, RHBG, COL15A1, PCDH12, IRS1, ASCC3, KIF16B, and MR1, as compared to the level of expression of the one or more genes in the subject prior to the administering.

In some embodiments of any of the methods described herein, the subject has been previously identified or diagnosed as having a liver disease or a metabolic syndrome. In some embodiments of any of the methods described herein, the subject has been previously identified or diagnosed as having a liver disease. In some embodiments of any of the metthods described herein, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments of any of the methods described herein, the subject has been previously identified or diagnosed as having a metabolic syndrome. In some embodiments of any of the methods described herein, the metabolic syndrome is selected from the group consisting of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

In some embodiments of any of the methods described herein, the first target-binding domain and the soluble tissue factor domain directly abut each other in the first chimeric polypeptide. In some embodiments of any of the methods described herein, the first chimeric polypeptide further comprises a linker sequence between the first target-binding domain and the soluble tissue factor domain in the first chimeric polypeptide. In some embodiments of any of the methods described herein, the soluble tissue factor domain and the first domain of the pair of affinity domains directly abut each other in the first chimeric polypeptide. In some embodiments of any of the methods described herein, the first chimeric polypeptide further comprises a linker sequence between the soluble tissue factor domain and the first domain of the pair of affinity domains in the first chimeric polypeptide.

In some embodiments of any of the methods described herein, the second domain of the pair of affinity domains and the second target-binding domain directly abut each other in the second chimeric polypeptide. In some embodiments of any of the methods described herein, the second chimeric polypeptide further comprises a linker sequence between the second domain of the pair of affinity domains and the second target-binding domain in the second chimeric polypeptide.

In some embodiments of any of the methods described herein, one or both of the first target-binding domain and the second target-binding domain is an antigen-binding domain. In some embodiments of any of the methods described herein, one or both of the first target-binding domain and the second target-binding domain is a soluble interleukin or cytokine receptor.

In some embodiments of any of the methods described herein, the first chimeric polypeptide further comprises one or more additional target-binding domain(s). In some embodiments of any of the methods described herein, the second chimeric polypeptide further comprises one or more additional target-binding domain(s).

In some embodiments of any of the methods described herein, the soluble tissue factor domain is a soluble human tissue factor domain. In some embodiments of any of the methods described herein, the soluble human tissue factor domain comprises a sequence that is at least 80% identical to SEQ ID NO: 1. In some embodiments of any of the methods described herein, the pair of affinity domains is a sushi domain from an alpha chain of human IL-15 receptor (IL-15Rα) and a soluble IL-15.

In some embodiments of any of the methods described herein, the first target-binding domain comprises a soluble TGF-PRII. In some embodiments of any of the methods described herein, the first target-binding domain comprises a first sequence that is at least 80% identical to SEQ ID NO: 2 and a second sequence that is at least 80% identical to SEQ ID NO: 2, wherein the first and second sequence are separated by a linker. In some embodiments of any of the methods described herein, the first target-binding domain comprises a first sequence that is at least 90% identical to SEQ ID NO: 2 and a second sequence that is at least 90% identical to SEQ ID NO: 2. In some embodiments of any of the methods described herein, the first target-binding domain comprises a first sequence of SEQ ID NO: 2 and a second sequence of SEQ ID NO: 2. In some embodiments of any of the methods described herein, the linker comprises a sequence of SEQ ID NO: 3. In some embodiments of any of the methods described herein, the first target-binding domain comprises a sequence that is at least 80% identical to SEQ ID NO: 4. In some embodiments of any of the methods described herein, the first target-binding domain comprises a sequence that is at least 90% identical to SEQ ID NO: 4. In some embodiments of any of the methods described herein, the first target-binding domain comprises a sequence of SEQ ID NO: 4. In some embodiments of any of the methods described herein, the first chimeric polypeptide comprises a sequence that is at least 80% identical to SEQ ID NO: 6. In some embodiments of any of the methods described herein, the first chimeric polypeptide comprises a sequence that is at least 90% identical to SEQ ID NO: 6. In some embodiments of any of the methods described herein, the first chimeric polypeptide comprises a sequence of SEQ ID NO: 6. In some embodiments of any of the methods described herein, the first chimeric polypeptide comprises a sequence of SEQ ID NO: 7.

In some embodiments of any of the methods described herein, the second target-binding domain comprises a soluble TGF-βRII. In some embodiments of any of the methods described herein, the second target-binding domain comprises a first sequence that is at least 80% identical to SEQ ID NO: 2 and a second sequence that is at least 80% identical to SEQ ID NO: 2, wherein the first and second sequence are separated by a linker. In some embodiments of any of the methods described herein, the second target-binding domain comprises a first sequence that is at least 90% identical to SEQ ID NO: 2 and a second sequence that is at least 90% identical to SEQ ID NO: 2. In some embodiments of any of the methods described herein, the second target-binding domain comprises a first sequence of SEQ ID NO: 2 and a second sequence of SEQ ID NO: 2. In some embodiments of any of the methods described herein, the linker comprises a sequence of SEQ ID NO: 3. In some embodiments of any of the methods described herein, the second target-binding domain comprises a sequence that is at least 80% identical to SEQ ID NO: 4. In some embodiments of any of the methods described herein, the second target-binding domain comprises a sequence that is at least 90% identical to SEQ ID NO: 4. In some embodiments of any of the methods described herein, the second target-binding domain comprises a sequence of SEQ ID NO: 4. In some embodiments of any of the methods described herein, the second chimeric polypeptide comprises a sequence that is at least 80% identical to SEQ ID NO: 5. In some embodiments of any of the methods described herein, the first chimeric polypeptide comprises a sequence that is at least 80% identical to SEQ ID NO: 6. In some embodiments of any of the methods described herein, the second chimeric polypeptide comprises a sequence that is at least 90% identical to SEQ ID NO: 5. In some embodiments of any of the methods described herein, the second chimeric polypeptide comprises a sequence of SEQ ID NO: 5. In some embodiments of any of the methods described herein, the first chimeric polypeptide comprises a sequence of SEQ ID NO: 6. In some embodiments of any of the methods described herein, the second chimeric polypeptide comprises a sequence of SEQ ID NO: 8.

As used herein, the term “chimeric” refers to a polypeptide that includes amino acid sequences (e.g., domains) originally derived from two different sources (e.g., two different naturally-occurring proteins, e.g., from the same or different species). For example, a chimeric polypeptide can include domains from at least two different naturally occurring human proteins. In some examples, a chimeric polypeptide can include a domain that is a synthetic sequence (e.g., an scFv) and a domain that is derived from a naturally-occurring protein (e.g., a naturally-occurring human protein). In some embodiments, a chimeric polypeptide can include at least two different domains that are synthetic sequences (e.g., two different scFvs).

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof In some embodiments, an antigen-binding domain can include an alternative scaffold. Non-limiting examples of antigen-binding domains are described herein. Additional examples of antigen-binding domains are known in the art.

A “soluble tissue factor domain” refers to a polypeptide having at least 70% identity (e.g., at least 75% identity, at least 80% identity, at least 85% identity, at least 90% identity, at least 95% identity, at least 99% identity, or 100% identical) to a segment of a wildtype mammalian tissue factor protein (e.g., a wildtype human tissue factor protein) that lacks the transmembrane domain and the intracellular domain. Non-limiting examples of soluble tissue factor domains are described herein.

The term “soluble interleukin receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble interleukin receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype interleukin receptor and retains its ability to specifically bind to one or more of its natural ligands, but lacks its transmembrane domain (and optionally, further lacks its intracellular domain). Non-limiting examples of soluble interleukin receptors are described herein.

The term “soluble cytokine receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble cytokine receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype cytokine receptor and retains its ability to specifically bind to one or more of its natural ligands, but lacks its transmembrane domain (and optionally, further lacks its intracellular domain). Non-limiting examples of soluble cytokine receptors are described herein.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, and multi-specific antibodies. One example of an antigen-binding domain is an antigen-binding domain formed by a VH-VL dimer. Additional examples of an antibody are described herein. Additional examples of an antibody are known in the art.

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an antigen-binding domain and an antigen or epitope. The affinity of a molecule X for its partner Y can be represented by the dissociation equilibrium constant (KD). The kinetic components that contribute to the dissociation equilibrium constant are described in more detail below. Affinity can be measured by common methods known in the art, including those described herein.

Affinity can be determined, for example, using surface plasmon resonance (SPR) technology (e.g., BIACORE®) or biolayer interferometry (e.g., FORTEBIO®). Additional methods for determining the affinity for an antigen-binding domain and its corresponding antigen or epitope are known in the art.

A “multi-chain polypeptide” as used herein to refers to a polypeptide comprising two or more (e.g., three, four, five, six, seven, eight, nine, or ten) protein chains (e.g., at least a first chimeric polypeptide and a second polypeptide), where the two or more proteins chains associate through non-covalent bonds to form a quaternary structure.

The term “pair of affinity domains” is two different protein domain(s) that bind specifically to each other with a K_{D} of less than of less than 1×10⁻⁷ M (e.g., less than 1×10⁻⁸ M, less than 1×10⁻⁹ M, less than 1×10⁻¹⁰ M, or less than 1×10⁻¹¹ M). In some examples, a pair of affinity domains can be a pair of naturally-occurring proteins. In some embodiments, a pair of affinity domains can be a pair of synthetic proteins. Non-limiting examples of pairs of affinity domains are described herein.

The term “epitope” means a portion of an antigen that specifically binds to an antigen-binding domain. Epitopes can, e.g., consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. Methods for identifying an epitope to which an antigen-binding domain binds are known in the art.

An “immune effector cell” refers to a cell of the immune system of a mammal that is capable, directly or indirectly, of recognizing and/or causing cytostasis or cell death of a pathogenic cell (e.g., a cancer cell) in the mammal. Non-limiting examples of immune effector cells include macrophages, T-lymphocytes (e.g., cytotoxic T-lymphocytes and T-helper cells), natural killer cells, neutrophils, monocytes, and eosinophils. Additional examples of immune effector cells are known in the art.

The term “treatment” means to ameliorate at least one symptom of a disorder. In some examples, the disorder being treated is cancer and to ameliorate at least one symptom of cancer includes reducing aberrant proliferation, gene expression, signaling, translation, and/or secretion of factors. Generally, the methods of treatment include administering a therapeutically effective amount of composition that reduces at least one symptom of a disorder to a subject who is in need of, or who has been determined to be in need of such treatment.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 shows exemplary diagrams for a multi-chain chimeric polypeptide: (i) a first chimeric polypeptide including a first target-binding domain (A), a soluble tissue factor domain, a first domain of an affinity pair of domains (soluble interleukin IL-15), and an additional target-binding domain (B); and (ii) second chimeric polypeptide including a second domain of an affinity pair of domains (IL-15 receptor alpha sushi domain), a second target-binding domain (C), and an additional antigen-binding domain (D). The top cartoon diagram depicts the association of the first and the second chimeric polypeptides through the pair of affinity domains. The bottom schematic diagrams show the order of the domains in the first and second chimeric polypeptides.

FIG. 2 shows exemplary diagrams for a multi-chain chimeric polypeptide: (i) a first chimeric polypeptide including a first target-binding domain (A), a soluble tissue factor domain including five amino acid substitutions in order to remove binding of the soluble tissue factor domain to FVIIa, a first domain of an affinity pair of domains (soluble interleukin IL-15 including a D8N or D8A amino acid substitution), and an additional target-binding domain (B); and (ii) second chimeric polypeptide including a second domain of an affinity pair of domains (IL-15 receptor alpha sushi domain), a second target-binding domain (C), and an additional antigen-binding domain (D). The top cartoon diagram depicts the association of the first and the second chimeric polypeptides through the pair of affinity domains. The bottom schematic diagrams show the order of the domains in the first and second chimeric polypeptides. In other embodiments of any of the multi-chain chimeric polypeptides described herein the soluble tissue factor domain can comprise or consists of a soluble wildtype human tissue factor domain (comprising or consisting of a contiguous sequence within wildtype human tissue factor).

FIG. 3 shows a schematic of the TGFRt15-TGFRs construct.

FIG. 4 shows an additional schematic of the TGFRt15-TGFRs construct.

FIG. 5 shows results of TGFβ1 inhibition by TGFRt15-TGFRs and TGFR-Fc.

FIG. 6 shows results of 32Dβ cell proliferation assay with TGFRt15-TGFRs or recombinant IL-15

FIGS. 7A and 7B show results of detecting IL-15 and TGFPRII in TGFRt15-TGFRs with corresponding antibodies using ELISA.

FIG. 8 is a line graph showing the chromatographic profile of TGFRt15-TGFRs protein containing cell culture supernatant following binding and elution on anti-TF antibody resin.

FIG. 9 shows the analytical SEC profile of TGFRt15-TGFRs.

FIG. 10 shows TGFRt15-TGFRs before and after deglycosylation as analyzed by reduced SDS-PAGE.

FIGS. 11A and 11B show spleen weight and the percentages of immune cell types in TGFRt15-TGFRs-treated and control-treated mice. FIG. 85A shows spleen weight in mice treated with TGFRt15-TGFRs as compared to PBS control. FIG. 85B shows the percentage of CD4+ T cells, CD8+ T cells, and NK cells in mice treated with TGFRt15-TGFRs as compared to PBS control.

FIG. 12A and 12B show the spleen weight and immunostimulation over 92 hours in mice treated with TGFRt15-TGFRs. FIG. 86A shows spleen weight of mice treated with TGFRt15-TGFRs at 16, 24, 48, 72, and 92 hours after treatment. FIG. 86B shows the percentages of immune cells in mice treated with TGFRt15-TGFRs at 16, 24, 48, 72, and 92 hours after treatment.

FIG. 13A and 13B show Ki67 and Granzyme B expression in mice treated with TGFRt15-TGFRs over time.

FIG. 14 shows enhancement of cytotoxicity of splenocytes by TGFRt15-TGFRs in C57BL/6 Mice.

FIG. 15 shows changes in tumor size in response to PBS treatment, chemotherapy alone, TGFRt15-TGFRs alone, or chemotherapy and TGFRt15-TGFRs combination, in a pancreatic cancer mouse model.

FIG. 16 shows the cytotoxicity of NK cells isolated from mice treated with TGFRt15-TGFRs.

FIGS. 17A-17C show in vivo stimulation of Tregs, NK cells, and CD8+ T cells in ApoE−/− mice fed with a Western diet and treated with TGFRt15-TGFRs.

FIGS. 18A-18C show immunostimulation in C57BL/6 mice following treatment with TGFRt15-TGFRs.

FIGS. 19A and 19B show in vivo induction of proliferation of NK cells and CD8+ T cells in ApoE−/− mice fed with a Western diet and treated with TGFRt15-TGFRs.

FIGS. 20A and 20B show enhancement of cytotoxicity of NK cells following treatment of NK cells with TGFRt15-TGFRs.

FIGS. 21A and 21B show enhancement of ADCC activity of NK cells following treatment of NK cells with TGFRt15-TGFRs.

FIGS. 22A-22H show antitumor activity of TGFRt15-TGFRs plus anti-TRP1 antibody (TA99) in combination with chemotherapy in a melanoma mouse model.

FIGS. 23A-23C show amelioration of the Western diet-induced hyperglycemia in ApoE−/− mice by TGFRt15-TGFRs.

FIG. 24 shows upregulation shows upregulation of CD44hi memory T cells upon treatment with TGFRt15-TGFRs.

FIG. 25 shows RNA-seq analysis of differentially expressed genes between the PBS (control group) or TGFRt15-TGFRs (TGFRt15-TGFRs group) in the liver of db/db mice.

FIG. 26 shows RNA-seq analysis of differentially expressed genes between the PBS (control group) or TGFRt15-TGFRs (TGFRt15-TGFRs group) in aged mice liver.

FIG. 27 shows a volcano plot of RNA-seq analysis of the livers of db/db mice. FIG. 28 shows a heatmap representing differentially expressed genes as measured by RNA-seq analysis of livers of db/db mice treated with TGFRt15-TGFRs (HCW9218) and PBS negative controls.

FIG. 29 shows a heat map of differentially expressed senescence-related and inflammation-related genes measured by RNA-seq in livers of aged mice.

FIG. 30 shows a schematic of a study design for investigating TGFRt15-TGFRs (HCW9218) treatment in a db/db mouse model.

FIG. 31 shows relative mRNA expression of IL1β, IL1α, PAI-1, IL6, and Tnfa in liver as measured by quantitative PCR after treatment with TGFRt15-TGFRs (HCW9218) compared to control at day 10 or day 60.

FIG. 32 shows relative mRNA expression of IL162 , IL1α, PAI-1, IL6, and Cdkn1a in liver after treatment with one or two TGFRt15-TGFRs (HCW9218) doses compared to control at day 120 as measured by quantitative PCR.

FIG. 33 shows ELISA data of protein levels of IL-1α, IL-6, IL-8, in liver tissue after treatment with TGFRt15-TGFRs (HCW9218) compared to control 120 days after treatment.

FIG. 34 shows ELISA data of protein levels of PAI-1, and Fibronectin in liver tissue after treatment with TGFRt15-TGFRs (HCW9218) compared to control 120 days after treatment.

FIG. 35 shows shows immunofluorescent staining of liver tissue cells expressing p21+after treatment with two doses of TGFRt15-TGFRs (HCW9218) compared to PBS negative control.

FIG. 36 shows heatmaps of differentially expressed genes as detected by RNA-seq data generated from the livers of aged mice receiving either TGFRt15-TGFRs (HCW9218) treatment of PBS-only negative control.

FIG. 37 shows flow cytometric analysis of Ki67 expression in CD4, CD8, Treg, and CD16+ NK cells in blood from Cynomolgus monkeys following treatment with TGFRt15-TGFRs (HCW9218).

FIG. 38 shows flow cytometric analysis of absolute numbers of CD4, CD8, Treg, and CD16+ NK cells in blood from Cynomolgus monkeys following treatment with

TGFRt15-TGFRs (HCW9218).

FIG. 39 shows TGFRt15-TGFRs treatment enhances immune cell populations in db/db mice.

FIG. 40 shows the effect of TGFRt15-TGFRs treatment or TGFRt15*-TGFRs treatment on cytotoxic activity of splenocytes in db/db mice after day 4 post-treatment.

FIG. 41 shows the effect of TGFRt15-TGFRs and TGFRt15*-TGFRs treatment on interferon-gamma production of splenocytes in db/db mice after day 4 post-treatment and in vitro αCD3/CD28 stimulation assays.

FIG. 42 shows the effect of TGFRt15-TGFRs on the glycolytic activity of splenocytes in db/db mice after day 4 post-treatment.

FIG. 43 shows the effect of TGFRt15-TGFRs on mitochondrial respiration of splenocytes in db/db mice after day 4 post-treatment.

FIG. 44 shows the effect of TGFRt15-TGFRs on plasma TGFβ1 and TGFIβ2 levels in db/db mice after day 4 post-treatment.

FIG. 45 shows the effect of TGFRt15-TGFRs (HCW9218) from chemical induced liver damages.

### DETAILED DESCRIPTION

Nonalcoholic fatty liver disease (NAFLD) is emerging as the leading chronic liver disease worldwide and is estimated to affect one billion individuals globally (Younossi et al., Hepatology 69(6):2672-2682, 2019). NAFLD represents a spectrum of liver diseases ranging from non-alcoholic fatty liver (NAFL), in the case of isolated steatosis to non-alcoholic steatohepatitis (NASH), fibrotic NASH, advanced fibrosis, cirrhosis and hepatocellular carcinoma (HCC) (Meijnikman et al., JHEP Rep. 3(4):100301, 2021).

The metabolic mechanism leading to NAFLD reflects an imbalance of energy metabolism in the liver. The inability of the liver to oxidize the excess energy, mostly in the form of carbohydrates and fat, to CO₂ or to export it as very-low-density lipoproteins. This leads to a net accumulation of energy in the liver as triglycerides, which explains the widespread presence of NAFLD in obese individuals and in individuals with lipodystrophy. Skeletal muscle insulin resistance, one of the earliest defects associated with metabolic syndrome and prediabetes, can also promote development of NAFLD through increase hepatic de novo lipogenesis (DNL) and hypertriglyceridemia by diverting ingested glucose away from skeletal muscles glycogen synthesis and into the liver for DNL. Development of hepatic insulin resistance, where insulin activation of glycogen synthase is impaired (Loomba et al., Cell 184(10):2537-2564, 2021), redirects glucose into lipogenic pathways and further promotes NAFLD.

Adipocyte dysfunction also promotes development of NAFLD. Severe NAFLD/NASH is a complication of congenital lipodystrophies, where the absence of adipose tissue forces the liver to store excess fatty acids, leading to severe insulin resistance. Other metabolic causes of hepatic steatosis include (1) defects in intrahepatic lipolysis, (2) defects in triglyceride export, (3) increased glucokinase activity resulting in hepatic DNL, and (4) reductions in hepatic mitochondrial/peroxisomal β-oxidation (Loomba et al., Cell 184(10):2537-2564, 2021).

Hepatocytes begin to amass fat when they synthesize new lipids through the DNL pathway, an adaptive response to counter the generation of toxic lipid metabolites and balance free fatty acid excess (Piccinin et al., Nat. Rev. Gastroenterol. Hepatol. 16(3):160-174, 2019). The accumulative toxic metabolites promote a hepatic inflammatory state that is further exacerbated by endotoxins derived from increased gut permeability and dysbiosis and release of IL-6 and TNF from inflamed adipose tissues (Loomba et al., Cell 184(10):2537-2564, 2021; Yki-Jarvinen et al., Nat. Rev. Gastroenterol. Hepatol. 2021). Thus, the hepatic inflammatory microenvironment plays a critical role in the development of NAFLD and progression toward HCC. In addition, cytokine mediated hepatocytes injury and death are followed by hepatic progenitor cell population growth, which, in an inflammatory environment, induces the fibrogenic response in hepatic stellate cells, thereby promoting progression toward liver fibrosis and NASH (Loomba et al., Cell 184(10):2537-2564, 2021). Recently, there is also evidence showing that hepatocyte cellular senescence is also a causal factor in NAFLD development.

NAFLD is associated with several markers of senescence in hepatocytes, such as increased senescence-associated damage foci, increased senescence-associated distention of satellites and larger nuclear areas (Ogrodnik et al., Nat. Comm. 8:15691, 2017).

Hepatocytic senescence was also shown to impair hepatic mitochondrial (3-oxidation, thereby hindering fatty acid elimination and promoting triglyceride accumulation (Ogrodnik et al., Nat. Comm. 8:15691, 2017). The paracrine effects of proinflammatory factors secreted from the senescence-associated secretory phenotype of senescent hepatocytes disrupt the normal metabolism of the normal hepatocytes (Loomba et al., Cell 184(10):2537-2564, 2021). Finally, a causal link between hepatocytic senescence and hepatic steatosis was unraveled using transgenic mice and a senolytic cocktail (Childs et al., Nat. Rev. Drug Discov. 16(10):718-735, 2017).

Herein, we describe that subcutaneous administration of TGFRt15-TGFRs can eliminate hepatocytic senescence, lower the inflammatory microenvironment of the liver, rebalance the metabolic functions of the liver, and reduce gluconeogenesis and lipogenesis in Db/Db and naturally-aging mouse models. Thus, TGFRt15-TGFRs has the potential to treat a variety of liver diseases and metabolic syndrome.

Provided herein are methods of treating a liver disease or a metabolic syndrome in a subject diagnosed as having the liver disease or the metabolic syndrome; methods of reducing one or more of the rate of progression from non-alcoholic fatty liver disease

(NAFL) to non-alcoholic steatohepatitis (NASH), progression from NASH to cirrhosis, and progression from cirrhosis to hepatocellular carcinoma; methods of reducing inflammation in a liver of a subject identified as being in need thereof; methods of decreasing gluconeogenesis in a liver of a subject identified as being in need thereof; methods of decreasing lipogenesis in a liver of a subject identified as being in need thereof; methods of decreasing hepatocytic senescence in a liver of a subject identified as being in need thereof; methods of rebalancing metabolic function in a liver of a subject identified as being in need thereof; and methods of modulating expression of one or more genes in Tables 1-4 in a liver of a subject identified as being in need thereof, that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGT-βRII) and the second target-binding domain binds specifically to a ligand of TGF-βRII.

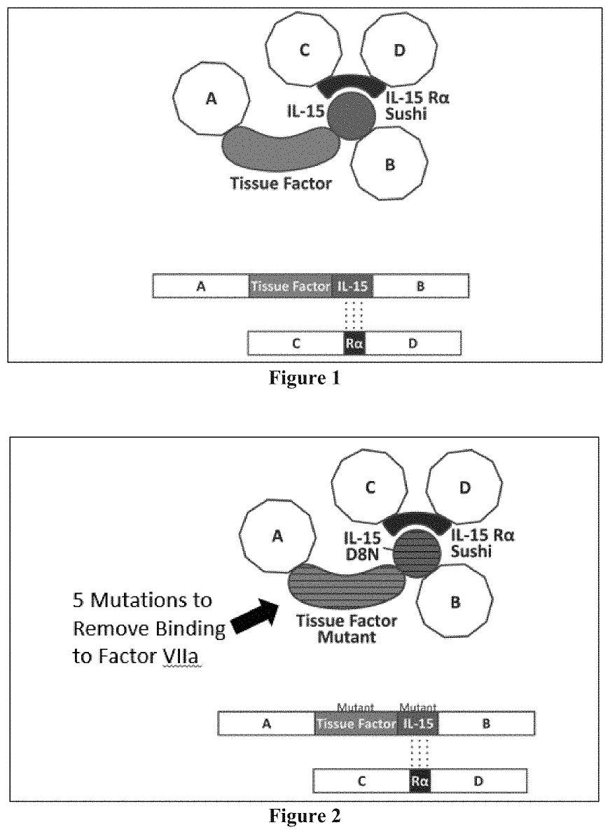

In some examples of any of the multi-chain chimeric polypeptides described herein the total length of first chimeric polypeptide and/or the second chimeric polypeptide can each independently be about 50 amino acids to about 3000 amino acids, about 50 amino acids to about 2500 amino acids, about 50 amino acids to about 2000 amino acids, about 50 amino acids to about 1500 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 480 amino acids, about 50 amino acids to about 460 amino acids, about 50 amino acids to about 440 amino acids, about 50 amino acids to about 420 amino acids, about 50 amino acids to about 400 amino acids, about 50 amino acids to about 380 amino acids, about 50 amino acids to about 360 amino acids, about 50 amino acids to about 340 amino acids, about 50 amino acids to about 320 amino acids, about 50 amino acids to about 300 amino acids, about 50 amino acids to about 280 amino acids, about 50 amino acids to about 260 amino acids, about 50 amino acids to about 240 amino acids, about 50 amino acids to about 220 amino acids, about 50 amino acids to about 200 amino acids, about 50 amino acids to about 150 amino acids, about 50 amino acids to about 100 amino acids, about 100 amino acids to about 3000 amino acids, about 100 amino acids to about 2500 amino acids, about 100 amino acids to about 2000 amino acids, about 100 amino acids to about 1500 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 480 amino acids, about 100 amino acids to about 460 amino acids, about 100 amino acids to about 440 amino acids, about 100 amino acids to about 420 amino acids, about 100 amino acids to about 400 amino acids, about 100 amino acids to about 380 amino acids, about 100 amino acids to about 360 amino acids, about 100 amino acids to about 340 amino acids, about 100 amino acids to about 320 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 150 amino acids, about 150 amino acids to about 3000 amino acids, about 150 amino acids to about 2500 amino acids, about 150 amino acids to about 2000 amino acids, about 150 amino acids to about 1500 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 480 amino acids, about 150 amino acids to about 460 amino acids, about 150 amino acids to about 440 amino acids, about 150 amino acids to about 420 amino acids, about 150 amino acids to about 400 amino acids, about 150 amino acids to about 380 amino acids, about 150 amino acids to about 360 amino acids, about 150 amino acids to about 340 amino acids, about 150 amino acids to about 320 amino acids, about 150 amino acids to about 300 amino acids, about 150 amino acids to about 280 amino acids, about 150 amino acids to about 260 amino acids, about 150 amino acids to about 240 amino acids, about 150 amino acids to about 220 amino acids, about 150 amino acids to about 200 amino acids, about 200 amino acids to about 3000 amino acids, about 200 amino acids to about 2500 amino acids, about 200 amino acids to about 2000 amino acids, about 200 amino acids to about 1500 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 480 amino acids, about 200 amino acids to about 460 amino acids, about 200 amino acids to about 440 amino acids, about 200 amino acids to about 420 amino acids, about 200 amino acids to about 400 amino acids, about 200 amino acids to about 380 amino acids, about 200 amino acids to about 360 amino acids, about 200 amino acids to about 340 amino acids, about 200 amino acids to about 320 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 3000 amino acids, about 220 amino acids to about 2500 amino acids, about 220 amino acids to about 2000 amino acids, about 220 amino acids to about 1500 amino acids, about 220 amino acids to about 1000 amino acids, about 220 amino acids to about 950 amino acids, about 220 amino acids to about 900 amino acids, about 220 amino acids to about 850 amino acids, about 220 amino acids to about 800 amino acids, about 220 amino acids to about 750 amino acids, about 220 amino acids to about 700 amino acids, about 220 amino acids to about 650 amino acids, about 220 amino acids to about 600 amino acids, about 220 amino acids to about 550 amino acids, about 220 amino acids to about 500 amino acids, about 220 amino acids to about 480 amino acids, about 220 amino acids to about 460 amino acids, about 220 amino acids to about 440 amino acids, about 220 amino acids to about 420 amino acids, about 220 amino acids to about 400 amino acids, about 220 amino acids to about 380 amino acids, about 220 amino acids to about 360 amino acids, about 220 amino acids to about 340 amino acids, about 220 amino acids to about 320 amino acids, about 220 amino acids to about 300 amino acids, about 220 amino acids to about 280 amino acids, about 220 amino acids to about 260 amino acids, about 220 amino acids to about 240 amino acids, about 240 amino acids to about 3000 amino acids, about 240 amino acids to about 2500 amino acids, about 240 amino acids to about 2000 amino acids, about 240 amino acids to about 1500 amino acids, about 240 amino acids to about 1000 amino acids, about 240 amino acids to about 950 amino acids, about 240 amino acids to about 900 amino acids, about 240 amino acids to about 850 amino acids, about 240 amino acids to about 800 amino acids, about 240 amino acids to about 750 amino acids, about 240 amino acids to about 700 amino acids, about 240 amino acids to about 650 amino acids, about 240 amino acids to about 600 amino acids, about 240 amino acids to about 550 amino acids, about 240 amino acids to about 500 amino acids, about 240 amino acids to about 480 amino acids, about 240 amino acids to about 460 amino acids, about 240 amino acids to about 440 amino acids, about 240 amino acids to about 420 amino acids, about 240 amino acids to about 400 amino acids, about 240 amino acids to about 380 amino acids, about 240 amino acids to about 360 amino acids, about 240 amino acids to about 340 amino acids, about 240 amino acids to about 320 amino acids, about 240 amino acids to about 300 amino acids, about 240 amino acids to about 280 amino acids, about 240 amino acids to about 260 amino acids, about 260 amino acids to about 3000 amino acids, about 260 amino acids to about 2500 amino acids, about 260 amino acids to about 2000 amino acids, about 260 amino acids to about 1500 amino acids, about 260 amino acids to about 1000 amino acids, about 260 amino acids to about 950 amino acids, about 260 amino acids to about 900 amino acids, about 260 amino acids to about 850 amino acids, about 260 amino acids to about 800 amino acids, about 260 amino acids to about 750 amino acids, about 260 amino acids to about 700 amino acids, about 260 amino acids to about 650 amino acids, about 260 amino acids to about 600 amino acids, about 260 amino acids to about 550 amino acids, about 260 amino acids to about 500 amino acids, about 260 amino acids to about 480 amino acids, about 260 amino acids to about 460 amino acids, about 260 amino acids to about 440 amino acids, about 260 amino acids to about 420 amino acids, about 260 amino acids to about 400 amino acids, about 260 amino acids to about 380 amino acids, about 260 amino acids to about 360 amino acids, about 260 amino acids to about 340 amino acids, about 260 amino acids to about 320 amino acids, about 260 amino acids to about 300 amino acids, about 260 amino acids to about 280 amino acids, about 280 amino acids to about 3000 amino acids, about 280 amino acids to about 2500 amino acids, about 280 amino acids to about 2000 amino acids, about 280 amino acids to about 1500 amino acids, about 280 amino acids to about 1000 amino acids, about 280 amino acids to about 950 amino acids, about 280 amino acids to about 900 amino acids, about 280 amino acids to about 850 amino acids, about 280 amino acids to about 800 amino acids, about 280 amino acids to about 750 amino acids, about 280 amino acids to about 700 amino acids, about 280 amino acids to about 650 amino acids, about 280 amino acids to about 600 amino acids, about 280 amino acids to about 550 amino acids, about 280 amino acids to about 500 amino acids, about 280 amino acids to about 480 amino acids, about 280 amino acids to about 460 amino acids, about 280 amino acids to about 440 amino acids, about 280 amino acids to about 420 amino acids, about 280 amino acids to about 400 amino acids, about 280 amino acids to about 380 amino acids, about 280 amino acids to about 360 amino acids, about 280 amino acids to about 340 amino acids, about 280 amino acids to about 320 amino acids, about 280 amino acids to about 300 amino acids, about 300 amino acids to about 3000 amino acids, about 300 amino acids to about 2500 amino acids, about 300 amino acids to about 2000 amino acids, about 300 amino acids to about 1500 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 480 amino acids, about 300 amino acids to about 460 amino acids, about 300 amino acids to about 440 amino acids, about 300 amino acids to about 420 amino acids, about 300 amino acids to about 400 amino acids, about 300 amino acids to about 380 amino acids, about 300 amino acids to about 360 amino acids, about 300 amino acids to about 340 amino acids, about 300 amino acids to about 320 amino acids, about 320 amino acids to about 3000 amino acids, about 320 amino acids to about 2500 amino acids, about 320 amino acids to about 2000 amino acids, about 320 amino acids to about 1500 amino acids, about 320 amino acids to about 1000 amino acids, about 320 amino acids to about 950 amino acids, about 320 amino acids to about 900 amino acids, about 320 amino acids to about 850 amino acids, about 320 amino acids to about 800 amino acids, about 320 amino acids to about 750 amino acids, about 320 amino acids to about 700 amino acids, about 320 amino acids to about 650 amino acids, about 320 amino acids to about 600 amino acids, about 320 amino acids to about 550 amino acids, about 320 amino acids to about 500 amino acids, about 320 amino acids to about 480 amino acids, about 320 amino acids to about 460 amino acids, about 320 amino acids to about 440 amino acids, about 320 amino acids to about 420 amino acids, about 320 amino acids to about 400 amino acids, about 320 amino acids to about 380 amino acids, about 320 amino acids to about 360 amino acids, about 320 amino acids to about 340 amino acids, about 340 amino acids to about 3000 amino acids, about 340 amino acids to about 2500 amino acids, about 340 amino acids to about 2000 amino acids, about 340 amino acids to about 1500 amino acids, about 340 amino acids to about 1000 amino acids, about 340 amino acids to about 950 amino acids, about 340 amino acids to about 900 amino acids, about 340 amino acids to about 850 amino acids, about 340 amino acids to about 800 amino acids, about 340 amino acids to about 750 amino acids, about 340 amino acids to about 700 amino acids, about 340 amino acids to about 650 amino acids, about 340 amino acids to about 600 amino acids, about 340 amino acids to about 550 amino acids, about 340 amino acids to about 500 amino acids, about 340 amino acids to about 480 amino acids, about 340 amino acids to about 460 amino acids, about 340 amino acids to about 440 amino acids, about 340 amino acids to about 420 amino acids, about 340 amino acids to about 400 amino acids, about 340 amino acids to about 380 amino acids, about 340 amino acids to about 360 amino acids, about 360 amino acids to about 3000 amino acids, about 360 amino acids to about 2500 amino acids, about 360 amino acids to about 2000 amino acids, about 360 amino acids to about 1500 amino acids, about 360 amino acids to about 1000 amino acids, about 360 amino acids to about 950 amino acids, about 360 amino acids to about 900 amino acids, about 360 amino acids to about 850 amino acids, about 360 amino acids to about 800 amino acids, about 360 amino acids to about 750 amino acids, about 360 amino acids to about 700 amino acids, about 360 amino acids to about 650 amino acids, about 360 amino acids to about 600 amino acids, about 360 amino acids to about 550 amino acids, about 360 amino acids to about 500 amino acids, about 360 amino acids to about 480 amino acids, about 360 amino acids to about 460 amino acids, about 360 amino acids to about 440 amino acids, about 360 amino acids to about 420 amino acids, about 360 amino acids to about 400 amino acids, about 360 amino acids to about 380 amino acids, about 380 amino acids to about 3000 amino acids, about 380 amino acids to about 2500 amino acids, about 380 amino acids to about 2000 amino acids, about 380 amino acids to about 1500 amino acids, about 380 amino acids to about 1000 amino acids, about 380 amino acids to about 950 amino acids, about 380 amino acids to about 900 amino acids, about 380 amino acids to about 850 amino acids, about 380 amino acids to about 800 amino acids, about 380 amino acids to about 750 amino acids, about 380 amino acids to about 700 amino acids, about 380 amino acids to about 650 amino acids, about 380 amino acids to about 600 amino acids, about 380 amino acids to about 550 amino acids, about 380 amino acids to about 500 amino acids, about 380 amino acids to about 480 amino acids, about 380 amino acids to about 460 amino acids, about 380 amino acids to about 440 amino acids, about 380 amino acids to about 420 amino acids, about 380 amino acids to about 400 amino acids, about 400 amino acids to about 3000 amino acids, about 400 amino acids to about 2500 amino acids, about 400 amino acids to about 2000 amino acids, about 400 amino acids to about 1500 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 480 amino acids, about 400 amino acids to about 460 amino acids, about 400 amino acids to about 440 amino acids, about 400 amino acids to about 420 amino acids, about 420 amino acids to about 3000 amino acids, about 420 amino acids to about 2500 amino acids, about 420 amino acids to about 2000 amino acids, about 420 amino acids to about 1500 amino acids, about 420 amino acids to about 1000 amino acids, about 420 amino acids to about 950 amino acids, about 420 amino acids to about 900 amino acids, about 420 amino acids to about 850 amino acids, about 420 amino acids to about 800 amino acids, about 420 amino acids to about 750 amino acids, about 420 amino acids to about 700 amino acids, about 420 amino acids to about 650 amino acids, about 420 amino acids to about 600 amino acids, about 420 amino acids to about 550 amino acids, about 420 amino acids to about 500 amino acids, about 420 amino acids to about 480 amino acids, about 420 amino acids to about 460 amino acids, about 420 amino acids to about 440 amino acids, about 440 amino acids to about 3000 amino acids, about 440 amino acids to about 2500 amino acids, about 440 amino acids to about 2000 amino acids, about 440 amino acids to about 1500 amino acids, about 440 amino acids to about 1000 amino acids, about 440 amino acids to about 950 amino acids, about 440 amino acids to about 900 amino acids, about 440 amino acids to about 850 amino acids, about 440 amino acids to about 800 amino acids, about 440 amino acids to about 750 amino acids, about 440 amino acids to about 700 amino acids, about 440 amino acids to about 650 amino acids, about 440 amino acids to about 600 amino acids, about 440 amino acids to about 550 amino acids, about 440 amino acids to about 500 amino acids, about 440 amino acids to about 480 amino acids, about 440 amino acids to about 460 amino acids, about 460 amino acids to about 3000 amino acids, about 460 amino acids to about 2500 amino acids, about 460 amino acids to about 2000 amino acids, about 460 amino acids to about 1500 amino acids, about 460 amino acids to about 1000 amino acids, about 460 amino acids to about 950 amino acids, about 460 amino acids to about 900 amino acids, about 460 amino acids to about 850 amino acids, about 460 amino acids to about 800 amino acids, about 460 amino acids to about 750 amino acids, about 460 amino acids to about 700 amino acids, about 460 amino acids to about 650 amino acids, about 460 amino acids to about 600 amino acids, about 460 amino acids to about 550 amino acids, about 460 amino acids to about 500 amino acids, about 460 amino acids to about 480 amino acids, about 480 amino acids to about 3000 amino acids, about 480 amino acids to about 2500 amino acids, about 480 amino acids to about 2000 amino acids, about 480 amino acids to about 1500 amino acids, about 480 amino acids to about 1000 amino acids, about 480 amino acids to about 950 amino acids, about 480 amino acids to about 900 amino acids, about 480 amino acids to about 850 amino acids, about 480 amino acids to about 800 amino acids, about 480 amino acids to about 750 amino acids, about 480 amino acids to about 700 amino acids, about 480 amino acids to about 650 amino acids, about 480 amino acids to about 600 amino acids, about 480 amino acids to about 550 amino acids, about 480 amino acids to about 500 amino acids, about 500 amino acids to about 3000 amino acids, about 500 amino acids to about 2500 amino acids, about 500 amino acids to about 2000 amino acids, about 500 amino acids to about 1500 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 3000 amino acids, about 550 amino acids to about 2500 amino acids, about 550 amino acids to about 2000 amino acids, about 550 amino acids to about 1500 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 3000 amino acids, about 600 amino acids to about 2500 amino acids, about 600 amino acids to about 2000 amino acids, about 600 amino acids to about 1500 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 3000 amino acids, about 650 amino acids to about 2500 amino acids, about 650 amino acids to about 2000 amino acids, about 650 amino acids to about 1500 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 3000 amino acids, about 700 amino acids to about 2500 amino acids, about 700 amino acids to about 2000 amino acids, about 700 amino acids to about 1500 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 3000 amino acids, about 750 amino acids to about 2500 amino acids, about 750 amino acids to about 2000 amino acids, about 750 amino acids to about 1500 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 3000 amino acids, about 800 amino acids to about 2500 amino acids, about 800 amino acids to about 2000 amino acids, about 800 amino acids to about 1500 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 3000 amino acids, about 850 amino acids to about 2500 amino acids, about 850 amino acids to about 2000 amino acids, about 850 amino acids to about 1500 amino acids, about 850 amino acids to about 1000 amino acids, about 850 amino acids to about 950 amino acids, about 850 amino acids to about 900 amino acids, about 900 amino acids to about 3000 amino acids, about 900 amino acids to about 2500 amino acids, about 900 amino acids to about 2000 amino acids, about 900 amino acids to about 1500 amino acids, about 900 amino acids to about 1000 amino acids, about 900 amino acids to about 950 amino acids, about 950 amino acids to about 3000 amino acids, about 950 amino acids to about 2500 amino acids, about 950 amino acids to about 2000 amino acids, about 950 amino acids to about 1500 amino acids, about 950 amino acids to about 1000 amino acids, about 1000 amino acids to about 3000 amino acids, about 1000 amino acids to about 2500 amino acids, about 1000 amino acids to about 2000 amino acids, about 1000 amino acids to about 1500 amino acids, about 1500 amino acids to about 3000 amino acids, about 1500 amino acids to about 2500 amino acids, about 1500 amino acids to about 2000 amino acids, about 2000 amino acids to about 3000 amino acids, about 2000 amino acids to about 2500 amino acids, or about 2500 amino acids to about 3000 amino acids. Diagrams of exemplary multi-chain chimeric polypeptides provided herein are depicted in FIGS. 1 and 2.

In some embodiments of any of the multi-chain chimeric polypeptides described herein, the first target-binding domain (e.g., any of the first target-binding domains described herein) and the soluble tissue factor domain (e.g., any of the exemplary soluble tissue factor domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the first target-binding domain (e.g., any of the exemplary first target-binding domains described herein) and the soluble tissue factor domain (e.g., any of the exemplary soluble tissue factor domains described herein) in the first chimeric polypeptide.

In some embodiments of any of the multi-chain chimeric polypeptides described herein, the soluble tissue factor domain (e.g., any of the exemplary soluble tissue factor domains described herein) and the first domain of the pair of affinity domains (e.g., any of the exemplary first domains of any of the exemplary pairs of affinity domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the soluble tissue factor domain (e.g., any of the exemplary soluble tissue factor domains described herein) and the first domain of the pair of affinity domains (e.g., any of the exemplary first domains of any of the exemplary pairs of affinity domains described herein) in the first chimeric polypeptide.

In some embodiments of any of the multi-chain chimeric polypeptides described herein, the second domain of the pair of affinity domains (e.g., any of the exemplary second domains of any of the exemplary pairs of affinity domains described herein) and the second target-binding domain (e.g., any of the exemplary second target-binding domains described herein) directly abut each other in the second chimeric polypeptide.

In some embodiments of any of the multi-chain chimeric polypeptides described herein, the second chimeric polypeptide further comprises a linker sequence (e.g., any of the exemplary linker sequences described herein or known in the art) between the second domain of the pair of affinity domains (e.g., any of the exemplary second domains of any of the exemplary pairs of affinity domains described herein) and the second target-binding domain (e.g., any of the exemplary second target-binding domains described herein) in the second chimeric polypeptide.

Non-limiting aspects of these chimeric polypeptides, nucleic acids, vectors, cells, and methods are described below, and can be used in any combination without limitation. Additional aspects of these chimeric polypeptides, nucleic acids, vectors, cells, and methods are known in the art.

### Tissue Factor

Human tissue factor is a 263 amino-acid transmembrane protein containing three domains: (1) a 219-amino acid N-terminal extracellular domain (residues 1-219); (2) a 22-amino acid transmembrane domain (residues 220-242); and (3) a 21-amino acid cytoplasmic C-terminal tail (residues 242-263) ((UniProtKB Identifier Number: P13726). The cytoplasmic tail contains two phosphorylation sites at Ser253 and Ser258, and one S-palmitoylation site at Cys245. Deletion or mutation of the cytoplasmic domain was not found to affect tissue factor coagulation activity. Tissue factor has one S-palmitoylation site in the intracellular domain of the protein at Cys245. The Cys245 is located at the amino acid terminus of the intracellular domain and close to the membrane surface. The tissue factor transmembrane domain is composed of a single-spanning a-helix.

The extracellular domain of tissue factor, composed of two fibronectin type III domains, is connected to the transmembrane domain through a six-amino acid linker. This linker provides conformational flexibility to decouple the tissue factor extracellular domain from its transmembrane and cytoplasmic domains. Each tissue factor fibronectin type III module is composed of two overlapping β sheets with the top sheet domain containing three antiparallel β-strands and the bottom sheet containing four β-strands. The β-strands are connected by β-loops between strand βA and βB, βC and βD, and βE and βF, all of which are conserved in conformation in the two modules. There are three short α-helix segments connecting the β-strands. A unique feature of tissue factor is a 17-amino acid α-hairpin between strand α10 and strand α11, which is not a common element of the fibronectin superfamily. The N-terminal domain also contains a 12 amino acid loop between β6F and β7G that is not present in the C-terminal domain and is unique to tissue factor. Such a fibronectin type III domain structure is a feature of the immunoglobulin-like family of protein folds and is conserved among a wide variety of extracellular proteins.

The zymogen FVII is rapidly converted to FVIIa by limited proteolysis once it binds to tissue to form the active tissue factor-FVIIa complex. The FVIIa, which circulates as an enzyme at a concentration of approximately 0.1 nM (1% of plasma FVII), can also bind directly to tissue factor. The allosteric interaction between tissue factor and FVIIa on the tissue factor-FVIIa complex greatly increases the enzymatic activity of FVIIa: an approximate 20- to 100-fold increase in the rate of hydrolysis of small, chromogenic peptidyl substrates, and nearly a million-fold increase in the rate of activation of the natural macromolecular substrates FIX and FX. In concert with allosteric activation of the active site of FVIIa upon binding to tissue factor, the formation of tissue factor-FVIIa complex on phospholipid bilayer (i.e., upon exposure of phosphatidyl-L-serine on membrane surfaces) increases the rate of FIX or FX activation, in a Ca²⁺-dependent manner, an additional 1,000-fold. The roughly million-fold overall increase in FX activation by tissue factor-FVIIa-phospholipid complex relative to free FVIIa is a critical regulatory point for the coagulation cascade.

FVII is a ˜50 kDa, single-chain polypeptide consisting of 406 amino acid residues, with an N-terminal γ-carboxyglutamate-rich (GLA) domain, two epidermal growth factor-like domains (EGF1 and EFG2), and a C-terminal serine protease domain. FVII is activated to FVIIa by a specific proteolytic cleavage of the Ile-¹⁵⁴-Arg¹⁵² bond in the short linker region between the EGF2 and the protease domain. This cleavage results in the light and heavy chains being held together by a single disulfide bond of Cys¹³⁵ and Cys²⁶². FVIIa binds phospholipid membrane in a Ca²⁺-dependent manner through its N-terminal GLA-domain. Immediately C-terminal to the GLA domain is an aromatic stack and two EGF domains. The aromatic stack connects the GLA to EGF1 domain which binds a single Ca²⁺ ion. Occupancy of this Ca²⁺-binding site increases FVIIa amidolytic activity and tissue factor association. The catalytic triad consist of His¹⁹³, Asp²⁴², and Ser³⁴⁴, and binding of a single Ca²⁺ ion within the FVIIa protease domain is critical for its catalytic activity. Proteolytic activation of FVII to FVIIa frees the newly formed amino terminus at Ile¹⁵³ to fold back and be inserted into the activation pocket forming a salt bridge with the carboxylate of Asp³⁴³ to generate the oxyanion hole. Formation of this salt bridge is critical for FVIIa activity. However, oxyanion hole formation does not occur in free FVIIa upon proteolytic activation. As a result, FVIIa circulates in a zymogen-like state that is poorly recognized by plasma protease inhibitors, allowing it to circulate with a half-life of approximately 90 minutes.

Tissue factor-mediated positioning of the FVIIa active site above the membrane surface is important for FVIIa towards cognate substrates. Free FVIIa adopts a stable, extended structure when bound to the membrane with its active site positioned ˜80 Å above the membrane surface. Upon FVIIa binding to tissue factor, the FVa active site is repositioned ˜6 Å closer to the membrane. This modulation may aid in a proper alignment of the FVIIa catalytic triad with the target substrate cleavage site. Using GLA-domainless FVIIa, it has been shown that the active site was still positioned a similar distance above the membrane, demonstrating that tissue factor is able to fully support FVIIa active site positioning even in the absence of FVIIa-membrane interaction. Additional data showed that tissue factor supported full FVIIa proteolytic activity as long as the tissue factor extracellular domain was tethered in some way to the membrane surface. However, raising the active site of FVIIa greater than 80 Å above the membrane surface greatly reduced the ability of the tissue factor-FVIIa complex to activate FX but did not diminish tissue factor-FVIIa amidolytic activity.

Alanine scanning mutagenesis has been used to assess the role of specific amino acid side chains in the tissue factor extracellular domain for interaction with FVIIa (Gibbs et al., Biochemistry 33(47): 14003-14010, 1994; Schullek et al., J Blot Chem 269(30): 19399-19403, 1994). Alanine substitution identified a limited number of residue positions at which alanine replacements cause 5- to 10-fold lower affinity for FVIIa binding. Most of these residue side chains were found to be well-exposed to solvent in the crystal structure, concordant with macromolecular ligand interaction. The FVIIa ligand-binding site is located over an extensive region at the boundary between the two modules. In the C-module, residues Arg¹³⁵ and Phe¹⁴⁰ located on the protruding B-C loop provide an independent contact with FVIIa. Leu¹³³ is located at the base of the fingerlike structure and packed into the cleft between the two modules. This provides continuity to a major cluster of important binding residues consisting of Lys²⁰, Thr⁶⁰, Asp⁵⁸, and Ile²². Thr⁶ is only partially solvent-exposed and may play a local structural role rather than making a significant contact with ligand. The binding site extends onto the concave side of the intermodule angle involving Glu²⁴ and Gln¹¹⁰, and potentially the more distant residue Val²⁰⁷. The binding region extends from Asp58 onto a convex surface area formed by Lys⁴⁸, Lys⁴⁶, Gln³⁷, Asp⁴⁴, and Trp⁴⁵. Trp⁴⁵ and Asp⁴⁴ do not interact independently with FVIIa, indicating that the mutational effect at the Trp⁴⁵ position may reflect a structural importance of this side chain for the local packing of the adjacent Asp⁴⁴ and Gln³⁷ side chain. The interactive area further includes two surface-exposed aromatic residues, Phe⁷⁶ and Tyr⁷⁸, which form part of the hydrophobic cluster in the N-module.

The known physiologic substrates of tissue factor-FVIIa are FVII, FIX, and FX and certain proteinase-activated receptors. Mutational analysis has identified a number of residues that, when mutated, support full FVIIa amidolytic activity towards small peptidyl substrates but are deficient in their ability to support macromolecular substrate (i.e., FVII, FIX, and FX) activation (Ruf et al., J Biol Chem 267(31): 22206-22210, 1992; Ruf et al., J Biol Chem 267(9): 6375-6381, 1992; Huang et al., J Biol Chem 271(36): 21752-21757, 1996; Kirchhofer et al., Biochemistry 39(25): 7380-7387, 2000). The tissue factor loop region at residues 159-165, and residues in or adjacent to this flexible loop have been shown to be critical for the proteolytic activity of the tissue factor-FVIIa complex. This defines the proposed substrate-binding exosite region of tissue factor that is quite distant from the FVIIa active site. A substitution of the glycine residue by a marginally bulkier residue alanine, significantly impairs tissue factor-FVIIa proteolytic activity. This suggests that the flexibility afforded by glycine is critical for the loop of residues 159-165 for tissue factor macromolecular substrate recognition.

The residues Lys¹⁶⁵ and Lys¹⁶⁶ have also been demonstrated to be important for substrate recognition and binding. Mutation of either of these residues to alanine results in a significant decrease in the tissue factor co-factor function. Lys¹⁶⁵ and Lys¹⁶⁶ face away from each other, with Lys¹⁶⁵ pointing towards FVIIa in most tissue factor-FVIIa structures, and Lys¹⁶⁶ pointing into the substrate binding exosite region in the crystal structure. Putative salt bridge formation between Lys¹⁶⁵ of and Gla³⁵ of FVIIa would support the notion that tissue factor interaction with the GLA domain of FVIIa modulates substrate recognition. These results suggest that the C-terminal portion of the tissue factor ectodomain directly interacts with the GLA-domain, the possible adjacent EGF1 domains, of FIX and FX, and that the presence of the FVIIa GLA-domain may modulate these interactions either directly or indirectly.

### Soluble Tissue Factor Domain

In some embodiments of any of the polypeptides, compositions, or methods described herein, the soluble tissue factor domain can be a wildtype tissue factor polypeptide lacking the signal sequence, the transmembrane domain, and the intracellular domain. In some examples, the soluble tissue factor domain can be a tissue factor mutant, wherein a wildtype tissue factor polypeptide lacking the signal sequence, the transmembrane domain, and the intracellular domain, and has been further modified at selected amino acids. In some examples, the soluble tissue factor domain can be a soluble human tissue factor domain. In some examples, the soluble tissue factor domain can be a soluble mouse tissue factor domain. In some examples, the soluble tissue factor domain can be a soluble rat tissue factor domain. Non-limiting examples of soluble human tissue factor domains, a mouse soluble tissue factor domain, a rat soluble tissue factor domain, and mutant soluble tissue factor domains are shown below.

Exemplary Soluble Human Tissue Factor Domain
(SEQ ID NO: 1)
SGTTNTVAAYNLTWKSTNFKTILEWEPKPVNQVYTVQISTKSGDWKSKC
FYTTDTECDLTDEIVKDVKQTYLARVFSYPAGNVESTGSAGEPLYENSP
EFTPYLETNLGQPTIQSFEQVGTKVNVTVEDERTLVRRNNTFLSLRDVF
GKDLIYTLYYWKSSSSGKKTAKTNTNEFLIDVDKGENYCFSVQAVIPSR
TVNRKSTDSPVECMGQEKGEFRE
Exemplary Nucleic Acid Encoding Soluble Human
Tissue Factor Domain
(SEQ ID NO: 9)
AGCGGCACAACCAACACAGTCGCTGCCTATAACCTCACTTGGAAGAGCA
CCAACTTCAAAACCATCCTCGAATGGGAACCCAAACCCGTTAACCAAGT
TTACACCGTGCAGATCAGCACCAAGTCCGGCGACTGGAAGTCCAAATGT
TTCTATACCACCGACACCGAGTGCGATCTCACCGATGAGATCGTGAAAG
ATGTGAAACAGACCTACCTCGCCCGGGTGTTTAGCTACCCCGCCGGCAA
TGTGGAGAGCACTGGTTCCGCTGGCGAGCCTTTATACGAGAACAGCCCC
GAATTTACCCCTTACCTCGAGACCAATTTAGGACAGCCCACCATCCAAA
GCTTTGAGCAAGTTGGCACAAAGGTGAATGTGACAGTGGAGGACGAGCG
GACTTTAGTGCGGCGGAACAACACCTTTCTCAGCCTCCGGGATGTGTTC
GGCAAAGATTTAATCTACACACTGTATTACTGGAAGTCCTCTTCCTCCG
GCAAGAAGACAGCTAAAACCAACACAAACGAGTTTTTAATCGACGTGGA
TAAAGGCGAAAACTACTGTTTCAGCGTGCAAGCTGTGATCCCCTCCCGG
ACCGTGAATAGGAAAAGCACCGATAGCCCCGTTGAGTGCATGGGCCAAG
AAAAGGGCGAGTTCCGGGAG
Exemplary Mutant Soluble Human Tissue Factor
Domain
(SEQ ID NO: 10)
SGTTNTVAAYNLTWKSTNFATALEWEPKPVNQVYTVQISTKSGDWKSKC
FYTTDTECALTDEIVKDVKQTYLARVFSYPAGNVESTGSAGEPLYENSP
EFTPYLETNLGQPTIQSFEQVGTKVNVTVEDERTLVARNNTALSLRDVF
GKDLIYTLYYWKSSSSGKKTAKTNTNEFLIDVDKGENYCFSVQAVIPSR
TVNRKSTDSPVECMGQEKGEFRE
Exemplary Mutant Soluble Human Tissue Factor
Domain
(SEQ ID NO: 11)
SGTTNTVAAYNLTWKSTNFATALEWEPKPVNQVYTVQISTKSGDAKSKC
FYTTDTECALTDEIVKDVKQTYLARVFSYPAGNVESTGSAGEPLAENSP
EFTPYLETNLGQPTIQSFEQVGTKVNVTVEDERTLVARNNTALSLRDVF
GKDLIYTLYYWKSSSSGKKTAKTNTNEFLIDVDKGENYCFSVQAVIPSR
TVNRKSTDSPVECMGQEKGEFRE
Exemplary Soluble Mouse Tissue Factor Domain
(SEQ ID NO: 12)
agipekafnltwistdfktilewqpkptnytytvqisdrsrnwknkcfs
ttdtecdltdeivkdvtwayeakvlsvprrnsvhgdgdqlvihgeeppf
tnapkflpyrdtnlgqpviqqfeqdgrklnvvvkdsltlvrkngtfltl
rqvfgkdlgyiityrkgsstgkktnitntnefsidveegvsycffvqam
ifsrktnqnspgsstveteqwksflge
Exemplary Soluble Rat Tissue Factor Domain
(SEQ ID NO: 13)
agtppgkafnltwistdfktilewqpkptnytytvqisdrsrnwkykct
gttdtecdltdeivkdvnwtyearvlsvpwrnsthgketlfgthgeepp
ftnarkfipyrdtkigqpviqkyeqggtklkvtvkdsftlvrkngtflt
lrqvfgndlgyiltyrkdsstgrktntthtneflidvekgvsycffaqa
vifsrktnhkspesitkcteqwksvlge

In some embodiments, a soluble tissue factor domain can include a sequence that is at least 70% identical, at least 72% identical, at least 74% identical, at least 76% 30 identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to SEQ ID NO: 1, 10, 11, 12, or 13. In some embodiments, a soluble tissue factor domain can include a sequence of SEQ ID NO: 1, 10, 11, 12, or 13, with one to twenty amino acids (e.g., 2, 3, 4, 5, 6, 7, 8, 9, 10, 11, 12, 13, 14, 15, 16, 17, 18, 19, or 20) amino acids removed from its N-terminus and/or one to twenty amino acids (e.g., 2, 3, 4, 5, 6, 7, 8, 9, 10, 11, 12, 13, 14, 15, 16, 17, 18, 19, or 20) amino acids removed from its C-terminus.

As can be appreciated in the art, one skilled in the art would understand that mutation of amino acids that are conserved between different mammalian species is more likely to decrease the activity and/or structural stability of the protein, while mutation of amino acids that are not conserved between different mammalian species is less likely to decrease the activity and/or structural stability of the protein.

In some examples of any of the multi-chain chimeric polypeptides described herein, the soluble tissue factor domain is not capable of binding to Factor VIIa. In some examples of any of the multi-chain chimeric polypeptides described herein, the soluble tissue factor domain does not convert inactive Factor X into Factor Xa. In some embodiments of any of the multi-chain chimeric polypeptides described herein, the multi-chain chimeric polypeptide does not stimulate blood coagulation in a mammal.

In some examples, the soluble tissue factor domain can be a soluble human tissue factor domain. In some embodiments, the soluble tissue factor domain can be a soluble mouse tissue factor domain. In some embodiments, the soluble tissue factor domain can be a soluble rat tissue factor domain.

In some examples, the soluble tissue factor domain does not include one or more (e.g., two, three, four, five, six, or seven) of: a lysine at an amino acid position that corresponds to amino acid position 20 of mature wildtype human tissue factor protein; an isoleucine at an amino acid position that corresponds to amino acid position 22 of mature wildtype human tissue factor protein; a tryptophan at an amino acid position that corresponds to amino acid position 45 of mature wildtype human tissue factor protein; an aspartic acid at an amino acid position that corresponds to amino acid position 58 of mature wildtype human tissue factor protein; a tyrosine at an amino acid position that corresponds to amino acid position 94 of mature wildtype human tissue factor protein; an arginine at an amino acid position that corresponds to amino acid position 135 of mature wildtype human tissue factor protein; and a phenylalanine at an amino acid position that corresponds to amino acid position 140 of mature wildtype human tissue factor protein. In some embodiments, the mutant soluble tissue factor possesses the amino acid sequence of SEQ ID NO: 10 or SEQ ID NO: 11.

In some examples, the soluble tissue factor domain can be encoded by a nucleic acid including a sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to SEQ ID NO: 9.

In some embodiments, the soluble tissue factor domain can have a total length of about 20 amino acids to about 220 amino acids, about 20 amino acids to about 215 amino acids, about 20 amino acids to about 210 amino acids, about 20 amino acids to about 205 amino acids, about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 175 amino acids, about 20 amino acids to about 170 amino acids, about 20 amino acids to about 165 amino acids, about 20 amino acids to about 160 amino acids, about 20 amino acids to about 155 amino acids, about 20 amino acids to about 150 amino acids, about 20 amino acids to about 145 amino acids, about 20 amino acids to about 140 amino acids, about 20 amino acids to about 135 amino acids, about 20 amino acids to about 130 amino acids, about 20 amino acids to about 125 amino acids, about 20 amino acids to about 120 amino acids, about 20 amino acids to about 115 amino acids, about 20 amino acids to about 110 amino acids, about 20 amino acids to about 105 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 60 amino acids, about 20 amino acids to about 50 amino acids, about 20 amino acids to about 40 amino acids, about 20 amino acids to about 30 amino acids, about 30 amino acids to about 220 amino acids, about 30 amino acids to about 215 amino acids, about 30 amino acids to about 210 amino acids, about 30 amino acids to about 205 amino acids, about 30 amino acids to about 200 amino acids, about 30 amino acids to about 195 amino acids, about 30 amino acids to about 190 amino acids, about 30 amino acids to about 185 amino acids, about 30 amino acids to about 180 amino acids, about 30 amino acids to about 175 amino acids, about 30 amino acids to about 170 amino acids, about 30 amino acids to about 165 amino acids, about 30 amino acids to about 160 amino acids, about 30 amino acids to about 155 amino acids, about 30 amino acids to about 150 amino acids, about 30 amino acids to about 145 amino acids, about 30 amino acids to about 140 amino acids, about 30 amino acids to about 135 amino acids, about 30 amino acids to about 130 amino acids, about 30 amino acids to about 125 amino acids, about 30 amino acids to about 120 amino acids, about 30 amino acids to about 115 amino acids, about 30 amino acids to about 110 amino acids, about 30 amino acids to about 105 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 60 amino acids, about 30 amino acids to about 50 amino acids, about 30 amino acids to about 40 amino acids, about 40 amino acids to about 220 amino acids, about 40 amino acids to about 215 amino acids, about 40 amino acids to about 210 amino acids, about 40 amino acids to about 205 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 175 amino acids, about 40 amino acids to about 170 amino acids, about 40 amino acids to about 165 amino acids, about 40 amino acids to about 160 amino acids, about 40 amino acids to about 155 amino acids, about 40 amino acids to about 150 amino acids, about 40 amino acids to about 145 amino acids, about 40 amino acids to about 140 amino acids, about 40 amino acids to about 135 amino acids, about 40 amino acids to about 130 amino acids, about 40 amino acids to about 125 amino acids, about 40 amino acids to about 120 amino acids, about 40 amino acids to about 115 amino acids, about 40 amino acids to about 110 amino acids, about 40 amino acids to about 105 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 60 amino acids, about 40 amino acids to about 50 amino acids, about 50 amino acids to about 220 amino acids, about 50 amino acids to about 215 amino acids, about 50 amino acids to about 210 amino acids, about 50 amino acids to about 205 amino acids, about 50 amino acids to about 200 amino acids, about 50 amino acids to about 195 amino acids, about 50 amino acids to about 190 amino acids, about 50 amino acids to about 185 amino acids, about 50 amino acids to about 180 amino acids, about 50 amino acids to about 175 amino acids, about 50 amino acids to about 170 amino acids, about 50 amino acids to about 165 amino acids, about 50 amino acids to about 160 amino acids, about 50 amino acids to about 155 amino acids, about 50 amino acids to about 150 amino acids, about 50 amino acids to about 145 amino acids, about 50 amino acids to about 140 amino acids, about 50 amino acids to about 135 amino acids, about 50 amino acids to about 130 amino acids, about 50 amino acids to about 125 amino acids, about 50 amino acids to about 120 amino acids, about 50 amino acids to about 115 amino acids, about 50 amino acids to about 110 amino acids, about 50 amino acids to about 105 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 60 amino acids, about 60 amino acids to about 220 amino acids, about 60 amino acids to about 215 amino acids, about 60 amino acids to about 210 amino acids, about 60 amino acids to about 205 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 175 amino acids, about 60 amino acids to about 170 amino acids, about 60 amino acids to about 165 amino acids, about 60 amino acids to about 160 amino acids, about 60 amino acids to about 155 amino acids, about 60 amino acids to about 150 amino acids, about 60 amino acids to about 145 amino acids, about 60 amino acids to about 140 amino acids, about 60 amino acids to about 135 amino acids, about 60 amino acids to about 130 amino acids, about 60 amino acids to about 125 amino acids, about 60 amino acids to about 120 amino acids, about 60 amino acids to about 115 amino acids, about 60 amino acids to about 110 amino acids, about 60 amino acids to about 105 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 70 amino acids to about 220 amino acids, about 70 amino acids to about 215 amino acids, about 70 amino acids to about 210 amino acids, about 70 amino acids to about 205 amino acids, about 70 amino acids to about 200 amino acids, about 70 amino acids to about 195 amino acids, about 70 amino acids to about 190 amino acids, about 70 amino acids to about 185 amino acids, about 70 amino acids to about 180 amino acids, about 70 amino acids to about 175 amino acids, about 70 amino acids to about 170 amino acids, about 70 amino acids to about 165 amino acids, about 70 amino acids to about 160 amino acids, about 70 amino acids to about 155 amino acids, about 70 amino acids to about 150 amino acids, about 70 amino acids to about 145 amino acids, about 70 amino acids to about 140 amino acids, about 70 amino acids to about 135 amino acids, about 70 amino acids to about 130 amino acids, about 70 amino acids to about 125 amino acids, about 70 amino acids to about 120 amino acids, about 70 amino acids to about 115 amino acids, about 70 amino acids to about 110 amino acids, about 70 amino acids to about 105 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 80 amino acids to about 220 amino acids, about 80 amino acids to about 215 amino acids, about 80 amino acids to about 210 amino acids, about 80 amino acids to about 205 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 175 amino acids, about 80 amino acids to about 170 amino acids, about 80 amino acids to about 165 amino acids, about 80 amino acids to about 160 amino acids, about 80 amino acids to about 155 amino acids, about 80 amino acids to about 150 amino acids, about 80 amino acids to about 145 amino acids, about 80 amino acids to about 140 amino acids, about 80 amino acids to about 135 amino acids, about 80 amino acids to about 130 amino acids, about 80 amino acids to about 125 amino acids, about 80 amino acids to about 120 amino acids, about 80 amino acids to about 115 amino acids, about 80 amino acids to about 110 amino acids, about 80 amino acids to about 105 amino acids, about 80 amino acids to about 100 amino acids, about 80 amino acids to about 95 amino acids, about 80 amino acids to about 90 amino acids, about 90 amino acids to about 220 amino acids, about 90 amino acids to about 215 amino acids, about 90 amino acids to about 210 amino acids, about 90 amino acids to about 205 amino acids, about 90 amino acids to about 200 amino acids, about 90 amino acids to about 195 amino acids, about 90 amino acids to about 190 amino acids, about 90 amino acids to about 185 amino acids, about 90 amino acids to about 180 amino acids, about 90 amino acids to about 175 amino acids, about 90 amino acids to about 170 amino acids, about 90 amino acids to about 165 amino acids, about 90 amino acids to about 160 amino acids, about 90 amino acids to about 155 amino acids, about 90 amino acids to about 150 amino acids, about 90 amino acids to about 145 amino acids, about 90 amino acids to about 140 amino acids, about 90 amino acids to about 135 amino acids, about 90 amino acids to about 130 amino acids, about 90 amino acids to about 125 amino acids, about 90 amino acids to about 120 amino acids, about 90 amino acids to about 115 amino acids, about 90 amino acids to about 110 amino acids, about 90 amino acids to about 105 amino acids, about 90 amino acids to about 100 amino acids, about 100 amino acids to about 220 amino acids, about 100 amino acids to about 215 amino acids, about 100 amino acids to about 210 amino acids, about 100 amino acids to about 205 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 175 amino acids, about 100 amino acids to about 170 amino acids, about 100 amino acids to about 165 amino acids, about 100 amino acids to about 160 amino acids, about 100 amino acids to about 155 amino acids, about 100 amino acids to about 150 amino acids, about 100 amino acids to about 145 amino acids, about 100 amino acids to about 140 amino acids, about 100 amino acids to about 135 amino acids, about 100 amino acids to about 130 amino acids, about 100 amino acids to about 125 amino acids, about 100 amino acids to about 120 amino acids, about 100 amino acids to about 115 amino acids, about 100 amino acids to about 110 amino acids, about 110 amino acids to about 220 amino acids, about 110 amino acids to about 215 amino acids, about 110 amino acids to about 210 amino acids, about 110 amino acids to about 205 amino acids, about 110 amino acids to about 200 amino acids, about 110 amino acids to about 195 amino acids, about 110 amino acids to about 190 amino acids, about 110 amino acids to about 185 amino acids, about 110 amino acids to about 180 amino acids, about 110 amino acids to about 175 amino acids, about 110 amino acids to about 170 amino acids, about 110 amino acids to about 165 amino acids, about 110 amino acids to about 160 amino acids, about 110 amino acids to about 155 amino acids, about 110 amino acids to about 150 amino acids, about 110 amino acids to about 145 amino acids, about 110 amino acids to about 140 amino acids, about 110 amino acids to about 135 amino acids, about 110 amino acids to about 130 amino acids, about 110 amino acids to about 125 amino acids, about 110 amino acids to about 120 amino acids, about 110 amino acids to about 115 amino acids, about 115 amino acids to about 220 amino acids, about 115 amino acids to about 215 amino acids, about 115 amino acids to about 210 amino acids, about 115 amino acids to about 205 amino acids, about 115 amino acids to about 200 amino acids, about 115 amino acids to about 195 amino acids, about 115 amino acids to about 190 amino acids, about 115 amino acids to about 185 amino acids, about 115 amino acids to about 180 amino acids, about 115 amino acids to about 175 amino acids, about 115 amino acids to about 170 amino acids, about 115 amino acids to about 165 amino acids, about 115 amino acids to about 160 amino acids, about 115 amino acids to about 155 amino acids, about 115 amino acids to about 150 amino acids, about 115 amino acids to about 145 amino acids, about 115 amino acids to about 140 amino acids, about 115 amino acids to about 135 amino acids, about 115 amino acids to about 130 amino acids, about 115 amino acids to about 125 amino acids, about 115 amino acids to about 120 amino acids, about 120 amino acids to about 220 amino acids, about 120 amino acids to about 215 amino acids, about 120 amino acids to about 210 amino acids, about 120 amino acids to about 205 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 175 amino acids, about 120 amino acids to about 170 amino acids, about 120 amino acids to about 165 amino acids, about 120 amino acids to about 160 amino acids, about 120 amino acids to about 155 amino acids, about 120 amino acids to about 150 amino acids, about 120 amino acids to about 145 amino acids, about 120 amino acids to about 140 amino acids, about 120 amino acids to about 135 amino acids, about 120 amino acids to about 130 amino acids, about 120 amino acids to about 125 amino acids, about 125 amino acids to about 220 amino acids, about 125 amino acids to about 215 amino acids, about 125 amino acids to about 210 amino acids, about 125 amino acids to about 205 amino acids, about 125 amino acids to about 200 amino acids, about 125 amino acids to about 195 amino acids, about 125 amino acids to about 190 amino acids, about 125 amino acids to about 185 amino acids, about 125 amino acids to about 180 amino acids, about 125 amino acids to about 175 amino acids, about 125 amino acids to about 170 amino acids, about 125 amino acids to about 165 amino acids, about 125 amino acids to about 160 amino acids, about 125 amino acids to about 155 amino acids, about 125 amino acids to about 150 amino acids, about 125 amino acids to about 145 amino acids, about 125 amino acids to about 140 amino acids, about 125 amino acids to about 135 amino acids, about 125 amino acids to about 130 amino acids, about 130 amino acids to about 220 amino acids, about 130 amino acids to about 215 amino acids, about 130 amino acids to about 210 amino acids, about 130 amino acids to about 205 amino acids, about 130 amino acids to about 200 amino acids, about 130 amino acids to about 195 amino acids, about 130 amino acids to about 190 amino acids, about 130 amino acids to about 185 amino acids, about 130 amino acids to about 180 amino acids, about 130 amino acids to about 175 amino acids, about 130 amino acids to about 170 amino acids, about 130 amino acids to about 165 amino acids, about 130 amino acids to about 160 amino acids, about 130 amino acids to about 155 amino acids, about 130 amino acids to about 150 amino acids, about 130 amino acids to about 145 amino acids, about 130 amino acids to about 140 amino acids, about 130 amino acids to about 135 amino acids, about 135 amino acids to about 220 amino acids, about 135 amino acids to about 215 amino acids, about 135 amino acids to about 210 amino acids, about 135 amino acids to about 205 amino acids, about 135 amino acids to about 200 amino acids, about 135 amino acids to about 195 amino acids, about 135 amino acids to about 190 amino acids, about 135 amino acids to about 185 amino acids, about 135 amino acids to about 180 amino acids, about 135 amino acids to about 175 amino acids, about 135 amino acids to about 170 amino acids, about 135 amino acids to about 165 amino acids, about 135 amino acids to about 160 amino acids, about 135 amino acids to about 155 amino acids, about 135 amino acids to about 150 amino acids, about 135 amino acids to about 145 amino acids, about 135 amino acids to about 140 amino acids, about 140 amino acids to about 220 amino acids, about 140 amino acids to about 215 amino acids, about 140 amino acids to about 210 amino acids, about 140 amino acids to about 205 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 175 amino acids, about 140 amino acids to about 170 amino acids, about 140 amino acids to about 165 amino acids, about 140 amino acids to about 160 amino acids, about 140 amino acids to about 155 amino acids, about 140 amino acids to about 150 amino acids, about 140 amino acids to about 145 amino acids, about 145 amino acids to about 220 amino acids, about 145 amino acids to about 215 amino acids, about 145 amino acids to about 210 amino acids, about 145 amino acids to about 205 amino acids, about 145 amino acids to about 200 amino acids, about 145 amino acids to about 195 amino acids, about 145 amino acids to about 190 amino acids, about 145 amino acids to about 185 amino acids, about 145 amino acids to about 180 amino acids, about 145 amino acids to about 175 amino acids, about 145 amino acids to about 170 amino acids, about 145 amino acids to about 165 amino acids, about 145 amino acids to about 160 amino acids, about 145 amino acids to about 155 amino acids, about 145 amino acids to about 150 amino acids, about 150 amino acids to about 220 amino acids, about 150 amino acids to about 215 amino acids, about 150 amino acids to about 210 amino acids, about 150 amino acids to about 205 amino acids, about 150 amino acids to about 200 amino acids, about 150 amino acids to about 195 amino acids, about 150 amino acids to about 190 amino acids, about 150 amino acids to about 185 amino acids, about 150 amino acids to about 180 amino acids, about 150 amino acids to about 175 amino acids, about 150 amino acids to about 170 amino acids, about 150 amino acids to about 165 amino acids, about 150 amino acids to about 160 amino acids, about 150 amino acids to about 155 amino acids, about 155 amino acids to about 220 amino acids, about 155 amino acids to about 215 amino acids, about 155 amino acids to about 210 amino acids, about 155 amino acids to about 205 amino acids, about 155 amino acids to about 200 amino acids, about 155 amino acids to about 195 amino acids, about 155 amino acids to about 190 amino acids, about 155 amino acids to about 185 amino acids, about 155 amino acids to about 180 amino acids, about 155 amino acids to about 175 amino acids, about 155 amino acids to about 170 amino acids, about 155 amino acids to about 165 amino acids, about 155 amino acids to about 160 amino acids, about 160 amino acids to about 220 amino acids, about 160 amino acids to about 215 amino acids, about 160 amino acids to about 210 amino acids, about 160 amino acids to about 205 amino acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 160 amino acids to about 175 amino acids, about 160 amino acids to about 170 amino acids, about 160 amino acids to about 165 amino acids, about 165 amino acids to about 220 amino acids, about 165 amino acids to about 215 amino acids, about 165 amino acids to about 210 amino acids, about 165 amino acids to about 205 amino acids, about 165 amino acids to about 200 amino acids, about 165 amino acids to about 195 amino acids, about 165 amino acids to about 190 amino acids, about 165 amino acids to about 185 amino acids, about 165 amino acids to about 180 amino acids, about 165 amino acids to about 175 amino acids, about 165 amino acids to about 170 amino acids, about 170 amino acids to about 220 amino acids, about 170 amino acids to about 215 amino acids, about 170 amino acids to about 210 amino acids, about 170 amino acids to about 205 amino acids, about 170 amino acids to about 200 amino acids, about 170 amino acids to about 195 amino acids, about 170 amino acids to about 190 amino acids, about 170 amino acids to about 185 amino acids, about 170 amino acids to about 180 amino acids, about 170 amino acids to about 175 amino acids, about 175 amino acids to about 220 amino acids, about 175 amino acids to about 215 amino acids, about 175 amino acids to about 210 amino acids, about 175 amino acids to about 205 amino acids, about 175 amino acids to about 200 amino acids, about 175 amino acids to about 195 amino acids, about 175 amino acids to about 190 amino acids, about 175 amino acids to about 185 amino acids, about 175 amino acids to about 180 amino acids, about 180 amino acids to about 220 amino acids, about 180 amino acids to about 215 amino acids, about 180 amino acids to about 210 amino acids, about 180 amino acids to about 205 amino acids, about 180 amino acids to about 200 amino acids, about 180 amino acids to about 195 amino acids, about 180 amino acids to about 190 amino acids, about 180 amino acids to about 185 amino acids, about 185 amino acids to about 220 amino acids, about 185 amino acids to about 215 amino acids, about 185 amino acids to about 210 amino acids, about 185 amino acids to about 205 amino acids, about 185 amino acids to about 200 amino acids, about 185 amino acids to about 195 amino acids, about 185 amino acids to about 190 amino acids, about 190 amino acids to about 220 amino acids, about 190 amino acids to about 215 amino acids, about 190 amino acids to about 210 amino acids, about 190 amino acids to about 205 amino acids, about 190 amino acids to about 200 amino acids, about 190 amino acids to about 195 amino acids, about 195 amino acids to about 220 amino acids, about 195 amino acids to about 215 amino acids, about 195 amino acids to about 210 amino acids, about 195 amino acids to about 205 amino acids, about 195 amino acids to about 200 amino acids, about 200 amino acids to about 220 amino acids, about 200 amino acids to about 215 amino acids, about 200 amino acids to about 210 amino acids, about 200 amino acids to about 205 amino acids, about 205 amino acids to about 220 amino acids, about 205 amino acids to about 215 amino acids, about 205 amino acids to about 210 amino acids, about 210 amino acids to about 220 amino acids, about 210 amino acids to about 215 amino acids, or about 215 amino acids to about 220 amino acids.

### Linker Sequences

In some embodiments, the linker sequence can be a flexible linker sequence. Non-limiting examples of linker sequences that can be used are described in Klein et al., Protein Engineering, Design & Selection 27(10):325-330, 2014; Priyanka et al., Protein Sci. 22(2):153-167, 2013. In some examples, the linker sequence is a synthetic linker sequence.

In some embodiments of any of the multi-chain chimeric polypeptides described herein, the first chimeric polypeptide can include one, two, three, four, five, six, seven, eight, nine, or ten linker sequence(s) (e.g., the same or different linker sequences, e.g., any of the exemplary linker sequences described herein or known in the art). In some embodiments of any of the multi-chain chimeric polypeptides described herein, the second chimeric polypeptide can include one, two, three, four, five, six, seven, eight, nine, or ten linker sequence(s) (e.g., the same or different linker sequences, e.g., any of the exemplary linker sequences described herein or known in the art).

In some embodiments, a linker sequence can have a total length of 1 amino acid to about 100 amino acids, 1 amino acid to about 90 amino acids, 1 amino acid to about 80 amino acids, 1 amino acid to about 70 amino acids, 1 amino acid to about 60 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 24 amino acids, 1 amino acid to about 22 amino acids, 1 amino acid to about 20 amino acids, 1 amino acid to about 18 amino acids, 1 amino acid to about 16 amino acids, 1 amino acid to about 14 amino acids, 1 amino acid to about 12 amino acids, 1 amino acid to about 10 amino acids, 1 amino acid to about 8 amino acids, 1 amino acid to about 6 amino acids, 1 amino acid to about 4 amino acids, about 2 amino acids to about 100 amino acids, about 2 amino acids to about 90 amino acids, about 2 amino acids to about 80 amino acids, about 2 amino acids to about 70 amino acids, about 2 amino acids to about 60 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 24 amino acids, about 2 amino acids to about 22 amino acids, about 2 amino acids to about 20 amino acids, about 2 amino acids to about 18 amino acids, about 2 amino acids to about 16 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 4 amino acids, about 4 amino acids to about 100 amino acids, about 4 amino acids to about 90 amino acids, about 4 amino acids to about 80 amino acids, about 4 amino acids to about 70 amino acids, about 4 amino acids to about 60 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 24 amino acids, about 4 amino acids to about 22 amino acids, about 4 amino acids to about 20 amino acids, about 4 amino acids to about 18 amino acids, about 4 amino acids to about 16 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 6 amino acids to about 100 amino acids, about 6 amino acids to about 90 amino acids, about 6 amino acids to about 80 amino acids, about 6 amino acids to about 70 amino acids, about 6 amino acids to about 60 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 24 amino acids, about 6 amino acids to about 22 amino acids, about 6 amino acids to about 20 amino acids, about 6 amino acids to about 18 amino acids, about 6 amino acids to about 16 amino acids, about 6 amino acids to about 14 amino acids, about 6 amino acids to about 12 amino acids, about 6 amino acids to about 10 amino acids, about 6 amino acids to about 8 amino acids, about 8 amino acids to about 100 amino acids, about 8 amino acids to about 90 amino acids, about 8 amino acids to about 80 amino acids, about 8 amino acids to about 70 amino acids, about 8 amino acids to about 60 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 24 amino acids, about 8 amino acids to about 22 amino acids, about 8 amino acids to about 20 amino acids, about 8 amino acids to about 18 amino acids, about 8 amino acids to about 16 amino acids, about 8 amino acids to about 14 amino acids, about 8 amino acids to about 12 amino acids, about 8 amino acids to about 10 amino acids, about 10 amino acids to about 100 amino acids, about 10 amino acids to about 90 amino acids, about 10 amino acids to about 80 amino acids, about 10 amino acids to about 70 amino acids, about 10 amino acids to about 60 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 24 amino acids, about 10 amino acids to about 22 amino acids, about 10 amino acids to about 20 amino acids, about 10 amino acids to about 18 amino acids, about 10 amino acids to about 16 amino acids, about 10 amino acids to about 14 amino acids, about 10 amino acids to about 12 amino acids, about 12 amino acids to about 100 amino acids, about 12 amino acids to about 90 amino acids, about 12 amino acids to about 80 amino acids, about 12 amino acids to about 70 amino acids, about 12 amino acids to about 60 amino acids, about 12 amino acids to about 50 amino acids, about 12 amino acids to about 45 amino acids, about 12 amino acids to about 40 amino acids, about 12 amino acids to about 35 amino acids, about 12 amino acids to about 30 amino acids, about 12 amino acids to about 25 amino acids, about 12 amino acids to about 24 amino acids, about 12 amino acids to about 22 amino acids, about 12 amino acids to about 20 amino acids, about 12 amino acids to about 18 amino acids, about 12 amino acids to about 16 amino acids, about 12 amino acids to about 14 amino acids, about 14 amino acids to about 100 amino acids, about 14 amino acids to about 90 amino acids, about 14 amino acids to about 80 amino acids, about 14 amino acids to about 70 amino acids, about 14 amino acids to about 60 amino acids, about 14 amino acids to about 50 amino acids, about 14 amino acids to about 45 amino acids, about 14 amino acids to about 40 amino acids, about 14 amino acids to about 35 amino acids, about 14 amino acids to about 30 amino acids, about 14 amino acids to about 25 amino acids, about 14 amino acids to about 24 amino acids, about 14 amino acids to about 22 amino acids, about 14 amino acids to about 20 amino acids, about 14 amino acids to about 18 amino acids, about 14 amino acids to about 16 amino acids, about 16 amino acids to about 100 amino acids, about 16 amino acids to about 90 amino acids, about 16 amino acids to about 80 amino acids, about 16 amino acids to about 70 amino acids, about 16 amino acids to about 60 amino acids, about 16 amino acids to about 50 amino acids, about 16 amino acids to about 45 amino acids, about 16 amino acids to about 40 amino acids, about 16 amino acids to about 35 amino acids, about 16 amino acids to about 30 amino acids, about 16 amino acids to about 25 amino acids, about 16 amino acids to about 24 amino acids, about 16 amino acids to about 22 amino acids, about 16 amino acids to about 20 amino acids, about 16 amino acids to about 18 amino acids, about 18 amino acids to about 100 amino acids, about 18 amino acids to about 90 amino acids, about 18 amino acids to about 80 amino acids, about 18 amino acids to about 70 amino acids, about 18 amino acids to about 60 amino acids, about 18 amino acids to about 50 amino acids, about 18 amino acids to about 45 amino acids, about 18 amino acids to about 40 amino acids, about 18 amino acids to about 35 amino acids, about 18 amino acids to about 30 amino acids, about 18 amino acids to about 25 amino acids, about 18 amino acids to about 24 amino acids, about 18 amino acids to about 22 amino acids, about 18 amino acids to about 20 amino acids, about 20 amino acids to about 100 amino acids, about 20 amino acids to about 90 amino acids, about 20 amino acids to about 80 amino acids, about 20 amino acids to about 70 amino acids, about 20 amino acids to about 60 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 20 amino acids to about 24 amino acids, about 20 amino acids to about 22 amino acids, about 22 amino acids to about 100 amino acids, about 22 amino acids to about 90 amino acids, about 22 amino acids to about 80 amino acids, about 22 amino acids to about 70 amino acids, about 22 amino acids to about 60 amino acids, about 22 amino acids to about 50 amino acids, about 22 amino acids to about 45 amino acids, about 22 amino acids to about 40 amino acids, about 22 amino acids to about 35 amino acids, about 22 amino acids to about 30 amino acids, about 22 amino acids to about 25 amino acids, about 22 amino acids to about 24 amino acids, about 25 amino acids to about 100 amino acids, about 25 amino acids to about 90 amino acids, about 25 amino acids to about 80 amino acids, about 25 amino acids to about 70 amino acids, about 25 amino acids to about 60 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 100 amino acids, about 30 amino acids to about 90 amino acids, about 30 amino acids to about 80 amino acids, about 30 amino acids to about 70 amino acids, about 30 amino acids to about 60 amino acids, about 30 amino acids to about 50 amino acids, about 30 amino acids to about 45 amino acids, about 30 amino acids to about 40 amino acids, about 30 amino acids to about 35 amino acids, about 35 amino acids to about 100 amino acids, about 35 amino acids to about 90 amino acids, about 35 amino acids to about 80 amino acids, about 35 amino acids to about 70 amino acids, about 35 amino acids to about 60 amino acids, about 35 amino acids to about 50 amino acids, about 35 amino acids to about 45 amino acids, about 35 amino acids to about 40 amino acids, about 40 amino acids to about 100 amino acids, about 40 amino acids to about 90 amino acids, about 40 amino acids to about 80 amino acids, about 40 amino acids to about 70 amino acids, about 40 amino acids to about 60 amino acids, about 40 amino acids to about 50 amino acids, about 40 amino acids to about 45 amino acids, about 45 amino acids to about 100 amino acids, about 45 amino acids to about 90 amino acids, about 45 amino acids to about 80 amino acids, about 45 amino acids to about 70 amino acids, about 45 amino acids to about 60 amino acids, about 45 amino acids to about 50 amino acids, about 50 amino acids to about 100 amino acids, about 50 amino acids to about 90 amino acids, about 50 amino acids to about 80 amino acids, about 50 amino acids to about 70 amino acids, about 50 amino acids to about 60 amino acids, about 60 amino acids to about 100 amino acids, about 60 amino acids to about 90 amino acids, about 60 amino acids to about 80 amino acids, about 60 amino acids to about 70 amino acids, about 70 amino acids to about 100 amino acids, about 70 amino acids to about 90 amino acids, about 70 amino acids to about 80 amino acids, about 80 amino acids to about 100 amino acids, about 80 amino acids to about 90 amino acids, or about 90 amino acids to about 100 amino acids.

In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs. In some embodiments, the linker has one or more Gly-Gly-Gly-Ser (GGGS) sequences, e.g., 1, 2, 3, 4, 5, 6, 7, 8, 9, or 10 or more GGGS sequences. In some embodiments, the linker has one or more Gly-Gly-Gly-Gly-Ser (GGGGS) sequences, e.g., 1, 2, 3, 4, 5, 6, 7, 8, 9, or 10 or more GGGGS sequences. In some embodiments, the linker has one or more Gly-Gly-Ser-Gly (GGSG) sequences, e.g., 1, 2, 3, 4, 5, 6, 7, 8, 9, or 10 or more GGSG sequences. In some embodiments, the linker sequence can comprise or consist of

GGGGSGGGGSGGGGS (SEQ ID NO: 3). In some embodiments, the linker sequence can be encoded by a nucleic acid comprising or consisting of: GGCGGTGGAGGATCCGGAGGAGGTGGCTCCGGCGGCGGAGGATCT (SEQ ID NO: 14). In some embodiments, the linker sequence can comprise or consist of:

(SEQ ID NO: 15)
GGGSGGGS

### Target-Binding Domains

In some embodiments of any of the multi-chain chimeric polypeptides described herein, the first target-binding domain, the second target-binding domain, and/or the additional one or more target-binding domains can be an antigen-binding domain that binds specifically to a ligand of TGF-PRII (e.g., any of the exemplary antigen-binding domains described herein or known in the art) or a soluble interleukin or cytokine receptor that binds specifically to a ligand of TGF-PRII (e.g., any of the exemplary soluble interleukin receptors or soluble cytokine receptors described herein).

In some embodiments of any of the multi-chain chimeric polypeptides described herein, the first target-binding domain, the second target-binding domain, and/or the one or more additional target-binding domains can each independent have a total number of amino acids of about 5 amino acids to about 1000 amino acids, about 5 amino acids to about 950 amino acids, about 5 amino acids to about 900 amino acids, about 5 amino acids to about 850 amino acids, about 5 amino acids to about 800 amino acids, about 5 amino acids to about 750 amino acids, about 5 amino acids to about 700 amino acids, about 5 amino acids to about 650 amino acids, about 5 amino acids to about 600 amino acids, about 5 amino acids to about 550 amino acids, about 5 amino acids to about 500 amino acids, about 5 amino acids to about 450 amino acids, about 5 amino acids to about 400 amino acids, about 5 amino acids to about 350 amino acids, about 5 amino acids to about 300 amino acids, about 5 amino acids to about 280 amino acids, about 5 amino acids to about 260 amino acids, about 5 amino acids to about 240 amino acids, about 5 amino acids to about 220 amino acids, about 5 amino acids to about 200 amino acids, about 5 amino acids to about 195 amino acids, about 5 amino acids to about 190 amino acids, about 5 amino acids to about 185 amino acids, about 5 amino acids to about 180 amino acids, about 5 amino acids to about 175 amino acids, about 5 amino acids to about 170 amino acids, about 5 amino acids to about 165 amino acids, about 5 amino acids to about 160 amino acids, about 5 amino acids to about 155 amino acids, about 5 amino acids to about 150 amino acids, about 5 amino acids to about 145 amino acids, about 5 amino acids to about 140 amino acids, about 5 amino acids to about 135 amino acids, about 5 amino acids to about 130 amino acids, about 5 amino acids to about 125 amino acids, about 5 amino acids to about 120 amino acids, about 5 amino acids to about 115 amino acids, about 5 amino acids to about 110 amino acids, about 5 amino acids to about 105 amino acids, about 5 amino acids to about 100 amino acids, about 5 amino acids to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 1000 amino acids, about 10 amino acids to about 950 amino acids, about 10 amino acids to about 900 amino acids, about 10 amino acids to about 850 amino acids, about 10 amino acids to about 800 amino acids, about 10 amino acids to about 750 amino acids, about 10 amino acids to about 700 amino acids, about 10 amino acids to about 650 amino acids, about 10 amino acids to about 600 amino acids, about 10 amino acids to about 550 amino acids, about 10 amino acids to about 500 amino acids, about 10 amino acids to about 450 amino acids, about 10 amino acids to about 400 amino acids, about 10 amino acids to about 350 amino acids, about 10 amino acids to about 300 amino acids, about 10 amino acids to about 280 amino acids, about 10 amino acids to about 260 amino acids, about 10 amino acids to about 240 amino acids, about 10 amino acids to about 220 amino acids, about 10 amino acids to about 200 amino acids, about 10 amino acids to about 195 amino acids, about 10 amino acids to about 190 amino acids, about 10 amino acids to about 185 amino acids, about 10 amino acids to about 180 amino acids, about 10 amino acids to about 175 amino acids, about 10 amino acids to about 170 amino acids, about 10 amino acids to about 165 amino acids, about 10 amino acids to about 160 amino acids, about 10 amino acids to about 155 amino acids, about 10 amino acids to about 150 amino acids, about 10 amino acids to about 145 amino acids, about 10 amino acids to about 140 amino acids, about 10 amino acids to about 135 amino acids, about 10 amino acids to about 130 amino acids, about 10 amino acids to about 125 amino acids, about 10 amino acids to about 120 amino acids, about 10 amino acids to about 115 amino acids, about 10 amino acids to about 110 amino acids, about 10 amino acids to about 105 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 15 amino acids to about 1000 amino acids, about 15 amino acids to about 950 amino acids, about 15 amino acids to about 900 amino acids, about 15 amino acids to about 850 amino acids, about 15 amino acids to about 800 amino acids, about 15 amino acids to about 750 amino acids, about 15 amino acids to about 700 amino acids, about 15 amino acids to about 650 amino acids, about 15 amino acids to about 600 amino acids, about 15 amino acids to about 550 amino acids, about 15 amino acids to about 500 amino acids, about 15 amino acids to about 450 amino acids, about 15 amino acids to about 400 amino acids, about 15 amino acids to about 350 amino acids, about 15 amino acids to about 300 amino acids, about 15 amino acids to about 280 amino acids, about 15 amino acids to about 260 amino acids, about 15 amino acids to about 240 amino acids, about 15 amino acids to about 220 amino acids, about 15 amino acids to about 200 amino acids, about 15 amino acids to about 195 amino acids, about 15 amino acids to about 190 amino acids, about 15 amino acids to about 185 amino acids, about 15 amino acids to about 180 amino acids, about 15 amino acids to about 175 amino acids, about 15 amino acids to about 170 amino acids, about 15 amino acids to about 165 amino acids, about 15 amino acids to about 160 amino acids, about 15 amino acids to about 155 amino acids, about 15 amino acids to about 150 amino acids, about 15 amino acids to about 145 amino acids, about 15 amino acids to about 140 amino acids, about 15 amino acids to about 135 amino acids, about 15 amino acids to about 130 amino acids, about 15 amino acids to about 125 amino acids, about 15 amino acids to about 120 amino acids, about 15 amino acids to about 115 amino acids, about 15 amino acids to about 110 amino acids, about 15 amino acids to about 105 amino acids, about 15 amino acids to about 100 amino acids, about 15 amino acids to about 95 amino acids, about 15 amino acids to about 90 amino acids, about 15 amino acids to about 85 amino acids, about 15 amino acids to about 80 amino acids, about 15 amino acids to about 75 amino acids, about 15 amino acids to about 70 amino acids, about 15 amino acids to about 65 amino acids, about 15 amino acids to about 60 amino acids, about 15 amino acids to about 55 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 1000 amino acids, about 20 amino acids to about 950 amino acids, about 20 amino acids to about 900 amino acids, about 20 amino acids to about 850 amino acids, about 20 amino acids to about 800 amino acids, about 20 amino acids to about 750 amino acids, about 20 amino acids to about 700 amino acids, about 20 amino acids to about 650 amino acids, about 20 amino acids to about 600 amino acids, about 20 amino acids to about 550 amino acids, about 20 amino acids to about 500 amino acids, about 20 amino acids to about 450 amino acids, about 20 amino acids to about 400 amino acids, about 20 amino acids to about 350 amino acids, about 20 amino acids to about 300 amino acids, about 20 amino acids to about 280 amino acids, about 20 amino acids to about 260 amino acids, about 20 amino acids to about 240 amino acids, about 20 amino acids to about 220 amino acids, about 20 amino acids to about 200 amino acids, about 20 amino acids to about 195 amino acids, about 20 amino acids to about 190 amino acids, about 20 amino acids to about 185 amino acids, about 20 amino acids to about 180 amino acids, about 20 amino acids to about 175 amino acids, about 20 amino acids to about 170 amino acids, about 20 amino acids to about 165 amino acids, about 20 amino acids to about 160 amino acids, about 20 amino acids to about 155 amino acids, about 20 amino acids to about 150 amino acids, about 20 amino acids to about 145 amino acids, about 20 amino acids to about 140 amino acids, about 20 amino acids to about 135 amino acids, about 20 amino acids to about 130 amino acids, about 20 amino acids to about 125 amino acids, about 20 amino acids to about 120 amino acids, about 20 amino acids to about 115 amino acids, about 20 amino acids to about 110 amino acids, about 20 amino acids to about 105 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 1000 amino acids, about 25 amino acids to about 950 amino acids, about 25 amino acids to about 900 amino acids, about 25 amino acids to about 850 amino acids, about 25 amino acids to about 800 amino acids, about 25 amino acids to about 750 amino acids, about 25 amino acids to about 700 amino acids, about 25 amino acids to about 650 amino acids, about 25 amino acids to about 600 amino acids, about 25 amino acids to about 550 amino acids, about 25 amino acids to about 500 amino acids, about 25 amino acids to about 450 amino acids, about 25 amino acids to about 400 amino acids, about 25 amino acids to about 350 amino acids, about 25 amino acids to about 300 amino acids, about 25 amino acids to about 280 amino acids, about 25 amino acids to about 260 amino acids, about 25 amino acids to about 240 amino acids, about 25 amino acids to about 220 amino acids, about 25 amino acids to about 200 amino acids, about 25 amino acids to about 195 amino acids, about 25 amino acids to about 190 amino acids, about 25 amino acids to about 185 amino acids, about 25 amino acids to about 180 amino acids, about 25 amino acids to about 175 amino acids, about 25 amino acids to about 170 amino acids, about 25 amino acids to about 165 amino acids, about 25 amino acids to about 160 amino acids, about 25 amino acids to about 155 amino acids, about 25 amino acids to about 150 amino acids, about 25 amino acids to about 145 amino acids, about 25 amino acids to about 140 amino acids, about 25 amino acids to about 135 amino acids, about 25 amino acids to about 130 amino acids, about 25 amino acids to about 125 amino acids, about 25 amino acids to about 120 amino acids, about 25 amino acids to about 115 amino acids, about 25 amino acids to about 110 amino acids, about 25 amino acids to about 105 amino acids, about 25 amino acids to about 100 amino acids, about 25 amino acids to about 95 amino acids, about 25 amino acids to about 90 amino acids, about 25 amino acids to about 85 amino acids, about 25 amino acids to about 80 amino acids, about 25 amino acids to about 75 amino acids, about 25 amino acids to about 70 amino acids, about 25 amino acids to about 65 amino acids, about 25 amino acids to about 60 amino acids, about 25 amino acids to about 55 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 1000 amino acids, about 30 amino acids to about 950 amino acids, about 30 amino acids to about 900 amino acids, about 30 amino acids to about 850 amino acids, about 30 amino acids to about 800 amino acids, about 30 amino acids to about 750 amino acids, about 30 amino acids to about 700 amino acids, about 30 amino acids to about 650 amino acids, about 30 amino acids to about 600 amino acids, about 30 amino acids to about 550 amino acids, about 30 amino acids to about 500 amino acids, about 30 amino acids to about 450 amino acids, about 30 amino acids to about 400 amino acids, about 30 amino acids to about 350 amino acids, about 30 amino acids to about 300 amino acids, about 30 amino acids to about 280 amino acids, about 30 amino acids to about 260 amino acids, about 30 amino acids to about 240 amino acids, about 30 amino acids to about 220 amino acids, about 30 amino acids to about 200 amino acids, about 30 amino acids to about 195 amino acids, about 30 amino acids to about 190 amino acids, about 30 amino acids to about 185 amino acids, about 30 amino acids to about 180 amino acids, about 30 amino acids to about 175 amino acids, about 30 amino acids to about 170 amino acids, about 30 amino acids to about 165 amino acids, about 30 amino acids to about 160 amino acids, about 30 amino acids to about 155 amino acids, about 30 amino acids to about 150 amino acids, about 30 amino acids to about 145 amino acids, about 30 amino acids to about 140 amino acids, about 30 amino acids to about 135 amino acids, about 30 amino acids to about 130 amino acids, about 30 amino acids to about 125 amino acids, about 30 amino acids to about 120 amino acids, about 30 amino acids to about 115 amino acids, about 30 amino acids to about 110 amino acids, about 30 amino acids to about 105 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 1000 amino acids, about 35 amino acids to about 950 amino acids, about 35 amino acids to about 900 amino acids, about 35 amino acids to about 850 amino acids, about 35 amino acids to about 800 amino acids, about 35 amino acids to about 750 amino acids, about 35 amino acids to about 700 amino acids, about 35 amino acids to about 650 amino acids, about 35 amino acids to about 600 amino acids, about 35 amino acids to about 550 amino acids, about 35 amino acids to about 500 amino acids, about 35 amino acids to about 450 amino acids, about 35 amino acids to about 400 amino acids, about 35 amino acids to about 350 amino acids, about 35 amino acids to about 300 amino acids, about 35 amino acids to about 280 amino acids, about 35 amino acids to about 260 amino acids, about 35 amino acids to about 240 amino acids, about 35 amino acids to about 220 amino acids, about 35 amino acids to about 200 amino acids, about 35 amino acids to about 195 amino acids, about 35 amino acids to about 190 amino acids, about 35 amino acids to about 185 amino acids, about 35 amino acids to about 180 amino acids, about 35 amino acids to about 175 amino acids, about 35 amino acids to about 170 amino acids, about 35 amino acids to about 165 amino acids, about 35 amino acids to about 160 amino acids, about 35 amino acids to about 155 amino acids, about 35 amino acids to about 150 amino acids, about 35 amino acids to about 145 amino acids, about 35 amino acids to about 140 amino acids, about 35 amino acids to about 135 amino acids, about 35 amino acids to about 130 amino acids, about 35 amino acids to about 125 amino acids, about 35 amino acids to about 120 amino acids, about 35 amino acids to about 115 amino acids, about 35 amino acids to about 110 amino acids, about 35 amino acids to about 105 amino acids, about 35 amino acids to about 100 amino acids, about 35 amino acids to about 95 amino acids, about 35 amino acids to about 90 amino acids, about 35 amino acids to about 85 amino acids, about 35 amino acids to about 80 amino acids, about 35 amino acids to about 75 amino acids, about 35 amino acids to about 70 amino acids, about 35 amino acids to about 65 amino acids, about 35 amino acids to about 60 amino acids, about 35 amino acids to about 55 amino acids, about 35 amino acids to about 50 amino acids, about 35 amino acids to about 45 amino acids, about 35 amino acids to about 40 amino acids, about 40 amino acids to about 1000 amino acids, about 40 amino acids to about 950 amino acids, about 40 amino acids to about 900 amino acids, about 40 amino acids to about 850 amino acids, about 40 amino acids to about 800 amino acids, about 40 amino acids to about 750 amino acids, about 40 amino acids to about 700 amino acids, about 40 amino acids to about 650 amino acids, about 40 amino acids to about 600 amino acids, about 40 amino acids to about 550 amino acids, about 40 amino acids to about 500 amino acids, about 40 amino acids to about 450 amino acids, about 40 amino acids to about 400 amino acids, about 40 amino acids to about 350 amino acids, about 40 amino acids to about 300 amino acids, about 40 amino acids to about 280 amino acids, about 40 amino acids to about 260 amino acids, about 40 amino acids to about 240 amino acids, about 40 amino acids to about 220 amino acids, about 40 amino acids to about 200 amino acids, about 40 amino acids to about 195 amino acids, about 40 amino acids to about 190 amino acids, about 40 amino acids to about 185 amino acids, about 40 amino acids to about 180 amino acids, about 40 amino acids to about 175 amino acids, about 40 amino acids to about 170 amino acids, about 40 amino acids to about 165 amino acids, about 40 amino acids to about 160 amino acids, about 40 amino acids to about 155 amino acids, about 40 amino acids to about 150 amino acids, about 40 amino acids to about 145 amino acids, about 40 amino acids to about 140 amino acids, about 40 amino acids to about 135 amino acids, about 40 amino acids to about 130 amino acids, about 40 amino acids to about 125 amino acids, about 40 amino acids to about 120 amino acids, about 40 amino acids to about 115 amino acids, about 40 amino acids to about 110 amino acids, about 40 amino acids to about 105 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 1000 amino acids, about 45 amino acids to about 950 amino acids, about 45 amino acids to about 900 amino acids, about 45 amino acids to about 850 amino acids, about 45 amino acids to about 800 amino acids, about 45 amino acids to about 750 amino acids, about 45 amino acids to about 700 amino acids, about 45 amino acids to about 650 amino acids, about 45 amino acids to about 600 amino acids, about 45 amino acids to about 550 amino acids, about 45 amino acids to about 500 amino acids, about 45 amino acids to about 450 amino acids, about 45 amino acids to about 400 amino acids, about 45 amino acids to about 350 amino acids, about 45 amino acids to about 300 amino acids, about 45 amino acids to about 280 amino acids, about 45 amino acids to about 260 amino acids, about 45 amino acids to about 240 amino acids, about 45 amino acids to about 220 amino acids, about 45 amino acids to about 200 amino acids, about 45 amino acids to about 195 amino acids, about 45 amino acids to about 190 amino acids, about 45 amino acids to about 185 amino acids, about 45 amino acids to about 180 amino acids, about 45 amino acids to about 175 amino acids, about 45 amino acids to about 170 amino acids, about 45 amino acids to about 165 amino acids, about 45 amino acids to about 160 amino acids, about 45 amino acids to about 155 amino acids, about 45 amino acids to about 150 amino acids, about 45 amino acids to about 145 amino acids, about 45 amino acids to about 140 amino acids, about 45 amino acids to about 135 amino acids, about 45 amino acids to about 130 amino acids, about 45 amino acids to about 125 amino acids, about 45 amino acids to about 120 amino acids, about 45 amino acids to about 115 amino acids, about 45 amino acids to about 110 amino acids, about 45 amino acids to about 105 amino acids, about 45 amino acids to about 100 amino acids, about 45 amino acids to about 95 amino acids, about 45 amino acids to about 90 amino acids, about 45 amino acids to about 85 amino acids, about 45 amino acids to about 80 amino acids, about 45 amino acids to about 75 amino acids, about 45 amino acids to about 70 amino acids, about 45 amino acids to about 65 amino acids, about 45 amino acids to about 60 amino acids, about 45 amino acids to about 55 amino acids, about 45 amino acids to about 50 amino acids, about 50 amino acids to about 1000 amino acids, about 50 amino acids to about 950 amino acids, about 50 amino acids to about 900 amino acids, about 50 amino acids to about 850 amino acids, about 50 amino acids to about 800 amino acids, about 50 amino acids to about 750 amino acids, about 50 amino acids to about 700 amino acids, about 50 amino acids to about 650 amino acids, about 50 amino acids to about 600 amino acids, about 50 amino acids to about 550 amino acids, about 50 amino acids to about 500 amino acids, about 50 amino acids to about 450 amino acids, about 50 amino acids to about 400 amino acids, about 50 amino acids to about 350 amino acids, about 50 amino acids to about 300 amino acids, about 50 amino acids to about 280 amino acids, about 50 amino acids to about 260 amino acids, about 50 amino acids to about 240 amino acids, about 50 amino acids to about 220 amino acids, about 50 amino acids to about 200 amino acids, about 50 amino acids to about 195 amino acids, about 50 amino acids to about 190 amino acids, about 50 amino acids to about 185 amino acids, about 50 amino acids to about 180 amino acids, about 50 amino acids to about 175 amino acids, about 50 amino acids to about 170 amino acids, about 50 amino acids to about 165 amino acids, about 50 amino acids to about 160 amino acids, about 50 amino acids to about 155 amino acids, about 50 amino acids to about 150 amino acids, about 50 amino acids to about 145 amino acids, about 50 amino acids to about 140 amino acids, about 50 amino acids to about 135 amino acids, about 50 amino acids to about 130 amino acids, about 50 amino acids to about 125 amino acids, about 50 amino acids to about 120 amino acids, about 50 amino acids to about 115 amino acids, about 50 amino acids to about 110 amino acids, about 50 amino acids to about 105 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 55 amino acids to about 1000 amino acids, about 55 amino acids to about 950 amino acids, about 55 amino acids to about 900 amino acids, about 55 amino acids to about 850 amino acids, about 55 amino acids to about 800 amino acids, about 55 amino acids to about 750 amino acids, about 55 amino acids to about 700 amino acids, about 55 amino acids to about 650 amino acids, about 55 amino acids to about 600 amino acids, about 55 amino acids to about 550 amino acids, about 55 amino acids to about 500 amino acids, about 55 amino acids to about 450 amino acids, about 55 amino acids to about 400 amino acids, about 55 amino acids to about 350 amino acids, about 55 amino acids to about 300 amino acids, about 55 amino acids to about 280 amino acids, about 55 amino acids to about 260 amino acids, about 55 amino acids to about 240 amino acids, about 55 amino acids to about 220 amino acids, about 55 amino acids to about 200 amino acids, about 55 amino acids to about 195 amino acids, about 55 amino acids to about 190 amino acids, about 55 amino acids to about 185 amino acids, about 55 amino acids to about 180 amino acids, about 55 amino acids to about 175 amino acids, about 55 amino acids to about 170 amino acids, about 55 amino acids to about 165 amino acids, about 55 amino acids to about 160 amino acids, about 55 amino acids to about 155 amino acids, about 55 amino acids to about 150 amino acids, about 55 amino acids to about 145 amino acids, about 55 amino acids to about 140 amino acids, about 55 amino acids to about 135 amino acids, about 55 amino acids to about 130 amino acids, about 55 amino acids to about 125 amino acids, about 55 amino acids to about 120 amino acids, about 55 amino acids to about 115 amino acids, about 55 amino acids to about 110 amino acids, about 55 amino acids to about 105 amino acids, about 55 amino acids to about 100 amino acids, about 55 amino acids to about 95 amino acids, about 55 amino acids to about 90 amino acids, about 55 amino acids to about 85 amino acids, about 55 amino acids to about 80 amino acids, about 55 amino acids to about 75 amino acids, about 55 amino acids to about 70 amino acids, about 55 amino acids to about 65 amino acids, about 55 amino acids to about 60 amino acids, about 60 amino acids to about 1000 amino acids, about 60 amino acids to about 950 amino acids, about 60 amino acids to about 900 amino acids, about 60 amino acids to about 850 amino acids, about 60 amino acids to about 800 amino acids, about 60 amino acids to about 750 amino acids, about 60 amino acids to about 700 amino acids, about 60 amino acids to about 650 amino acids, about 60 amino acids to about 600 amino acids, about 60 amino acids to about 550 amino acids, about 60 amino acids to about 500 amino acids, about 60 amino acids to about 450 amino acids, about 60 amino acids to about 400 amino acids, about 60 amino acids to about 350 amino acids, about 60 amino acids to about 300 amino acids, about 60 amino acids to about 280 amino acids, about 60 amino acids to about 260 amino acids, about 60 amino acids to about 240 amino acids, about 60 amino acids to about 220 amino acids, about 60 amino acids to about 200 amino acids, about 60 amino acids to about 195 amino acids, about 60 amino acids to about 190 amino acids, about 60 amino acids to about 185 amino acids, about 60 amino acids to about 180 amino acids, about 60 amino acids to about 175 amino acids, about 60 amino acids to about 170 amino acids, about 60 amino acids to about 165 amino acids, about 60 amino acids to about 160 amino acids, about 60 amino acids to about 155 amino acids, about 60 amino acids to about 150 amino acids, about 60 amino acids to about 145 amino acids, about 60 amino acids to about 140 amino acids, about 60 amino acids to about 135 amino acids, about 60 amino acids to about 130 amino acids, about 60 amino acids to about 125 amino acids, about 60 amino acids to about 120 amino acids, about 60 amino acids to about 115 amino acids, about 60 amino acids to about 110 amino acids, about 60 amino acids to about 105 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 65 amino acids to about 1000 amino acids, about 65 amino acids to about 950 amino acids, about 65 amino acids to about 900 amino acids, about 65 amino acids to about 850 amino acids, about 65 amino acids to about 800 amino acids, about 65 amino acids to about 750 amino acids, about 65 amino acids to about 700 amino acids, about 65 amino acids to about 650 amino acids, about 65 amino acids to about 600 amino acids, about 65 amino acids to about 550 amino acids, about 65 amino acids to about 500 amino acids, about 65 amino acids to about 450 amino acids, about 65 amino acids to about 400 amino acids, about 65 amino acids to about 350 amino acids, about 65 amino acids to about 300 amino acids, about 65 amino acids to about 280 amino acids, about 65 amino acids to about 260 amino acids, about 65 amino acids to about 240 amino acids, about 65 amino acids to about 220 amino acids, about 65 amino acids to about 200 amino acids, about 65 amino acids to about 195 amino acids, about 65 amino acids to about 190 amino acids, about 65 amino acids to about 185 amino acids, about 65 amino acids to about 180 amino acids, about 65 amino acids to about 175 amino acids, about 65 amino acids to about 170 amino acids, about 65 amino acids to about 165 amino acids, about 65 amino acids to about 160 amino acids, about 65 amino acids to about 155 amino acids, about 65 amino acids to about 150 amino acids, about 65 amino acids to about 145 amino acids, about 65 amino acids to about 140 amino acids, about 65 amino acids to about 135 amino acids, about 65 amino acids to about 130 amino acids, about 65 amino acids to about 125 amino acids, about 65 amino acids to about 120 amino acids, about 65 amino acids to about 115 amino acids, about 65 amino acids to about 110 amino acids, about 65 amino acids to about 105 amino acids, about 65 amino acids to about 100 amino acids, about 65 amino acids to about 95 amino acids, about 65 amino acids to about 90 amino acids, about 65 amino acids to about 85 amino acids, about 65 amino acids to about 80 amino acids, about 65 amino acids to about 75 amino acids, about 65 amino acids to about 70 amino acids, about 70 amino acids to about 1000 amino acids, about 70 amino acids to about 950 amino acids, about 70 amino acids to about 900 amino acids, about 70 amino acids to about 850 amino acids, about 70 amino acids to about 800 amino acids, about 70 amino acids to about 750 amino acids, about 70 amino acids to about 700 amino acids, about 70 amino acids to about 650 amino acids, about 70 amino acids to about 600 amino acids, about 70 amino acids to about 550 amino acids, about 70 amino acids to about 500 amino acids, about 70 amino acids to about 450 amino acids, about 70 amino acids to about 400 amino acids, about 70 amino acids to about 350 amino acids, about 70 amino acids to about 300 amino acids, about 70 amino acids to about 280 amino acids, about 70 amino acids to about 260 amino acids, about 70 amino acids to about 240 amino acids, about 70 amino acids to about 220 amino acids, about 70 amino acids to about 200 amino acids, about 70 amino acids to about 195 amino acids, about 70 amino acids to about 190 amino acids, about 70 amino acids to about 185 amino acids, about 70 amino acids to about 180 amino acids, about 70 amino acids to about 175 amino acids, about 70 amino acids to about 170 amino acids, about 70 amino acids to about 165 amino acids, about 70 amino acids to about 160 amino acids, about 70 amino acids to about 155 amino acids, about 70 amino acids to about 150 amino acids, about 70 amino acids to about 145 amino acids, about 70 amino acids to about 140 amino acids, about 70 amino acids to about 135 amino acids, about 70 amino acids to about 130 amino acids, about 70 amino acids to about 125 amino acids, about 70 amino acids to about 120 amino acids, about 70 amino acids to about 115 amino acids, about 70 amino acids to about 110 amino acids, about 70 amino acids to about 105 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 70 amino acids to about 75 amino acids, about 75 amino acids to about 1000 amino acids, about 75 amino acids to about 950 amino acids, about 75 amino acids to about 900 amino acids, about 75 amino acids to about 850 amino acids, about 75 amino acids to about 800 amino acids, about 75 amino acids to about 750 amino acids, about 75 amino acids to about 700 amino acids, about 75 amino acids to about 650 amino acids, about 75 amino acids to about 600 amino acids, about 75 amino acids to about 550 amino acids, about 75 amino acids to about 500 amino acids, about 75 amino acids to about 450 amino acids, about 75 amino acids to about 400 amino acids, about 75 amino acids to about 350 amino acids, about 75 amino acids to about 300 amino acids, about 75 amino acids to about 280 amino acids, about 75 amino acids to about 260 amino acids, about 75 amino acids to about 240 amino acids, about 75 amino acids to about 220 amino acids, about 75 amino acids to about 200 amino acids, about 75 amino acids to about 195 amino acids, about 75 amino acids to about 190 amino acids, about 75 amino acids to about 185 amino acids, about 75 amino acids to about 180 amino acids, about 75 amino acids to about 175 amino acids, about 75 amino acids to about 170 amino acids, about 75 amino acids to about 165 amino acids, about 75 amino acids to about 160 amino acids, about 75 amino acids to about 155 amino acids, about 75 amino acids to about 150 amino acids, about 75 amino acids to about 145 amino acids, about 75 amino acids to about 140 amino acids, about 75 amino acids to about 135 amino acids, about 75 amino acids to about 130 amino acids, about 75 amino acids to about 125 amino acids, about 75 amino acids to about 120 amino acids, about 75 amino acids to about 115 amino acids, about 75 amino acids to about 110 amino acids, about 75 amino acids to about 105 amino acids, about 75 amino acids to about 100 amino acids, about 75 amino acids to about 95 amino acids, about 75 amino acids to about 90 amino acids, about 75 amino acids to about 85 amino acids, about 75 amino acids to about 80 amino acids, about 80 amino acids to about 1000 amino acids, about 80 amino acids to about 950 amino acids, about 80 amino acids to about 900 amino acids, about 80 amino acids to about 850 amino acids, about 80 amino acids to about 800 amino acids, about 80 amino acids to about 750 amino acids, about 80 amino acids to about 700 amino acids, about 80 amino acids to about 650 amino acids, about 80 amino acids to about 600 amino acids, about 80 amino acids to about 550 amino acids, about 80 amino acids to about 500 amino acids, about 80 amino acids to about 450 amino acids, about 80 amino acids to about 400 amino acids, about 80 amino acids to about 350 amino acids, about 80 amino acids to about 300 amino acids, about 80 amino acids to about 280 amino acids, about 80 amino acids to about 260 amino acids, about 80 amino acids to about 240 amino acids, about 80 amino acids to about 220 amino acids, about 80 amino acids to about 200 amino acids, about 80 amino acids to about 195 amino acids, about 80 amino acids to about 190 amino acids, about 80 amino acids to about 185 amino acids, about 80 amino acids to about 180 amino acids, about 80 amino acids to about 175 amino acids, about 80 amino acids to about 170 amino acids, about 80 amino acids to about 165 amino acids, about 80 amino acids to about 160 amino acids, about 80 amino acids to about 155 amino acids, about 80 amino acids to about 150 amino acids, about 80 amino acids to about 145 amino acids, about 80 amino acids to about 140 amino acids, about 80 amino acids to about 135 amino acids, about 80 amino acids to about 130 amino acids, about 80 amino acids to about 125 amino acids, about 80 amino acids to about 120 amino acids, about 80 amino acids to about 115 amino acids, about 80 amino acids to about 110 amino acids, about 80 amino acids to about 105 amino acids, about 80 amino acids to about 100 amino acids, about 80 amino acids to about 95 amino acids, about 80 amino acids to about 90 amino acids, about 80 amino acids to about 85 amino acids, about 85 amino acids to about 1000 amino acids, about 85 amino acids to about 950 amino acids, about 85 amino acids to about 900 amino acids, about 85 amino acids to about 850 amino acids, about 85 amino acids to about 800 amino acids, about 85 amino acids to about 750 amino acids, about 85 amino acids to about 700 amino acids, about 85 amino acids to about 650 amino acids, about 85 amino acids to about 600 amino acids, about 85 amino acids to about 550 amino acids, about 85 amino acids to about 500 amino acids, about 85 amino acids to about 450 amino acids, about 85 amino acids to about 400 amino acids, about 85 amino acids to about 350 amino acids, about 85 amino acids to about 300 amino acids, about 85 amino acids to about 280 amino acids, about 85 amino acids to about 260 amino acids, about 85 amino acids to about 240 amino acids, about 85 amino acids to about 220 amino acids, about 85 amino acids to about 200 amino acids, about 85 amino acids to about 195 amino acids, about 85 amino acids to about 190 amino acids, about 85 amino acids to about 185 amino acids, about 85 amino acids to about 180 amino acids, about 85 amino acids to about 175 amino acids, about 85 amino acids to about 170 amino acids, about 85 amino acids to about 165 amino acids, about 85 amino acids to about 160 amino acids, about 85 amino acids to about 155 amino acids, about 85 amino acids to about 150 amino acids, about 85 amino acids to about 145 amino acids, about 85 amino acids to about 140 amino acids, about 85 amino acids to about 135 amino acids, about 85 amino acids to about 130 amino acids, about 85 amino acids to about 125 amino acids, about 85 amino acids to about 120 amino acids, about 85 amino acids to about 115 amino acids, about 85 amino acids to about 110 amino acids, about 85 amino acids to about 105 amino acids, about 85 amino acids to about 100 amino acids, about 85 amino acids to about 95 amino acids, about 85 amino acids to about 90 amino acids, about 90 amino acids to about 1000 amino acids, about 90 amino acids to about 950 amino acids, about 90 amino acids to about 900 amino acids, about 90 amino acids to about 850 amino acids, about 90 amino acids to about 800 amino acids, about 90 amino acids to about 750 amino acids, about 90 amino acids to about 700 amino acids, about 90 amino acids to about 650 amino acids, about 90 amino acids to about 600 amino acids, about 90 amino acids to about 550 amino acids, about 90 amino acids to about 500 amino acids, about 90 amino acids to about 450 amino acids, about 90 amino acids to about 400 amino acids, about 90 amino acids to about 350 amino acids, about 90 amino acids to about 300 amino acids, about 90 amino acids to about 280 amino acids, about 90 amino acids to about 260 amino acids, about 90 amino acids to about 240 amino acids, about 90 amino acids to about 220 amino acids, about 90 amino acids to about 200 amino acids, about 90 amino acids to about 195 amino acids, about 90 amino acids to about 190 amino acids, about 90 amino acids to about 185 amino acids, about 90 amino acids to about 180 amino acids, about 90 amino acids to about 175 amino acids, about 90 amino acids to about 170 amino acids, about 90 amino acids to about 165 amino acids, about 90 amino acids to about 160 amino acids, about 90 amino acids to about 155 amino acids, about 90 amino acids to about 150 amino acids, about 90 amino acids to about 145 amino acids, about 90 amino acids to about 140 amino acids, about 90 amino acids to about 135 amino acids, about 90 amino acids to about 130 amino acids, about 90 amino acids to about 125 amino acids, about 90 amino acids to about 120 amino acids, about 90 amino acids to about 115 amino acids, about 90 amino acids to about 110 amino acids, about 90 amino acids to about 105 amino acids, about 90 amino acids to about 100 amino acids, about 90 amino acids to about 95 amino acids, about 95 amino acids to about 1000 amino acids, about 95 amino acids to about 950 amino acids, about 95 amino acids to about 900 amino acids, about 95 amino acids to about 850 amino acids, about 95 amino acids to about 800 amino acids, about 95 amino acids to about 750 amino acids, about 95 amino acids to about 700 amino acids, about 95 amino acids to about 650 amino acids, about 95 amino acids to about 600 amino acids, about 95 amino acids to about 550 amino acids, about 95 amino acids to about 500 amino acids, about 95 amino acids to about 450 amino acids, about 95 amino acids to about 400 amino acids, about 95 amino acids to about 350 amino acids, about 95 amino acids to about 300 amino acids, about 95 amino acids to about 280 amino acids, about 95 amino acids to about 260 amino acids, about 95 amino acids to about 240 amino acids, about 95 amino acids to about 220 amino acids, about 95 amino acids to about 200 amino acids, about 95 amino acids to about 195 amino acids, about 95 amino acids to about 190 amino acids, about 95 amino acids to about 185 amino acids, about 95 amino acids to about 180 amino acids, about 95 amino acids to about 175 amino acids, about 95 amino acids to about 170 amino acids, about 95 amino acids to about 165 amino acids, about 95 amino acids to about 160 amino acids, about 95 amino acids to about 155 amino acids, about 95 amino acids to about 150 amino acids, about 95 amino acids to about 145 amino acids, about 95 amino acids to about 140 amino acids, about 95 amino acids to about 135 amino acids, about 95 amino acids to about 130 amino acids, about 95 amino acids to about 125 amino acids, about 95 amino acids to about 120 amino acids, about 95 amino acids to about 115 amino acids, about 95 amino acids to about 110 amino acids, about 95 amino acids to about 105 amino acids, about 95 amino acids to about 100 amino acids, about 100 amino acids to about 1000 amino acids, about 100 amino acids to about 950 amino acids, about 100 amino acids to about 900 amino acids, about 100 amino acids to about 850 amino acids, about 100 amino acids to about 800 amino acids, about 100 amino acids to about 750 amino acids, about 100 amino acids to about 700 amino acids, about 100 amino acids to about 650 amino acids, about 100 amino acids to about 600 amino acids, about 100 amino acids to about 550 amino acids, about 100 amino acids to about 500 amino acids, about 100 amino acids to about 450 amino acids, about 100 amino acids to about 400 amino acids, about 100 amino acids to about 350 amino acids, about 100 amino acids to about 300 amino acids, about 100 amino acids to about 280 amino acids, about 100 amino acids to about 260 amino acids, about 100 amino acids to about 240 amino acids, about 100 amino acids to about 220 amino acids, about 100 amino acids to about 200 amino acids, about 100 amino acids to about 195 amino acids, about 100 amino acids to about 190 amino acids, about 100 amino acids to about 185 amino acids, about 100 amino acids to about 180 amino acids, about 100 amino acids to about 175 amino acids, about 100 amino acids to about 170 amino acids, about 100 amino acids to about 165 amino acids, about 100 amino acids to about 160 amino acids, about 100 amino acids to about 155 amino acids, about 100 amino acids to about 150 amino acids, about 100 amino acids to about 145 amino acids, about 100 amino acids to about 140 amino acids, about 100 amino acids to about 135 amino acids, about 100 amino acids to about 130 amino acids, about 100 amino acids to about 125 amino acids, about 100 amino acids to about 120 amino acids, about 100 amino acids to about 115 amino acids, about 100 amino acids to about 110 amino acids, about 100 amino acids to about 105 amino acids, about 105 amino acids to about 1000 amino acids, about 105 amino acids to about 950 amino acids, about 105 amino acids to about 900 amino acids, about 105 amino acids to about 850 amino acids, about 105 amino acids to about 800 amino acids, about 105 amino acids to about 750 amino acids, about 105 amino acids to about 700 amino acids, about 105 amino acids to about 650 amino acids, about 105 amino acids to about 600 amino acids, about 105 amino acids to about 550 amino acids, about 105 amino acids to about 500 amino acids, about 105 amino acids to about 450 amino acids, about 105 amino acids to about 400 amino acids, about 105 amino acids to about 350 amino acids, about 105 amino acids to about 300 amino acids, about 105 amino acids to about 280 amino acids, about 105 amino acids to about 260 amino acids, about 105 amino acids to about 240 amino acids, about 105 amino acids to about 220 amino acids, about 105 amino acids to about 200 amino acids, about 105 amino acids to about 195 amino acids, about 105 amino acids to about 190 amino acids, about 105 amino acids to about 185 amino acids, about 105 amino acids to about 180 amino acids, about 105 amino acids to about 175 amino acids, about 105 amino acids to about 170 amino acids, about 105 amino acids to about 165 amino acids, about 105 amino acids to about 160 amino acids, about 105 amino acids to about 155 amino acids, about 105 amino acids to about 150 amino acids, about 105 amino acids to about 145 amino acids, about 105 amino acids to about 140 amino acids, about 105 amino acids to about 135 amino acids, about 105 amino acids to about 130 amino acids, about 105 amino acids to about 125 amino acids, about 105 amino acids to about 120 amino acids, about 105 amino acids to about 115 amino acids, about 105 amino acids to about 110 amino acids, about 110 amino acids to about 1000 amino acids, about 110 amino acids to about 950 amino acids, about 110 amino acids to about 900 amino acids, about 110 amino acids to about 850 amino acids, about 110 amino acids to about 800 amino acids, about 110 amino acids to about 750 amino acids, about 110 amino acids to about 700 amino acids, about 110 amino acids to about 650 amino acids, about 110 amino acids to about 600 amino acids, about 110 amino acids to about 550 amino acids, about 110 amino acids to about 500 amino acids, about 110 amino acids to about 450 amino acids, about 110 amino acids to about 400 amino acids, about 110 amino acids to about 350 amino acids, about 110 amino acids to about 300 amino acids, about 110 amino acids to about 280 amino acids, about 110 amino acids to about 260 amino acids, about 110 amino acids to about 240 amino acids, about 110 amino acids to about 220 amino acids, about 110 amino acids to about 200 amino acids, about 110 amino acids to about 195 amino acids, about 110 amino acids to about 190 amino acids, about 110 amino acids to about 185 amino acids, about 110 amino acids to about 180 amino acids, about 110 amino acids to about 175 amino acids, about 110 amino acids to about 170 amino acids, about 110 amino acids to about 165 amino acids, about 110 amino acids to about 160 amino acids, about 110 amino acids to about 155 amino acids, about 110 amino acids to about 150 amino acids, about 110 amino acids to about 145 amino acids, about 110 amino acids to about 140 amino acids, about 110 amino acids to about 135 amino acids, about 110 amino acids to about 130 amino acids, about 110 amino acids to about 125 amino acids, about 110 amino acids to about 120 amino acids, about 110 amino acids to about 115 amino acids, about 115 amino acids to about 1000 amino acids, about 115 amino acids to about 950 amino acids, about 115 amino acids to about 900 amino acids, about 115 amino acids to about 850 amino acids, about 115 amino acids to about 800 amino acids, about 115 amino acids to about 750 amino acids, about 115 amino acids to about 700 amino acids, about 115 amino acids to about 650 amino acids, about 115 amino acids to about 600 amino acids, about 115 amino acids to about 550 amino acids, about 115 amino acids to about 500 amino acids, about 115 amino acids to about 450 amino acids, about 115 amino acids to about 400 amino acids, about 115 amino acids to about 350 amino acids, about 115 amino acids to about 300 amino acids, about 115 amino acids to about 280 amino acids, about 115 amino acids to about 260 amino acids, about 115 amino acids to about 240 amino acids, about 115 amino acids to about 220 amino acids, about 115 amino acids to about 200 amino acids, about 115 amino acids to about 195 amino acids, about 115 amino acids to about 190 amino acids, about 115 amino acids to about 185 amino acids, about 115 amino acids to about 180 amino acids, about 115 amino acids to about 175 amino acids, about 115 amino acids to about 170 amino acids, about 115 amino acids to about 165 amino acids, about 115 amino acids to about 160 amino acids, about 115 amino acids to about 155 amino acids, about 115 amino acids to about 150 amino acids, about 115 amino acids to about 145 amino acids, about 115 amino acids to about 140 amino acids, about 115 amino acids to about 135 amino acids, about 115 amino acids to about 130 amino acids, about 115 amino acids to about 125 amino acids, about 115 amino acids to about 120 amino acids, about 120 amino acids to about 1000 amino acids, about 120 amino acids to about 950 amino acids, about 120 amino acids to about 900 amino acids, about 120 amino acids to about 850 amino acids, about 120 amino acids to about 800 amino acids, about 120 amino acids to about 750 amino acids, about 120 amino acids to about 700 amino acids, about 120 amino acids to about 650 amino acids, about 120 amino acids to about 600 amino acids, about 120 amino acids to about 550 amino acids, about 120 amino acids to about 500 amino acids, about 120 amino acids to about 450 amino acids, about 120 amino acids to about 400 amino acids, about 120 amino acids to about 350 amino acids, about 120 amino acids to about 300 amino acids, about 120 amino acids to about 280 amino acids, about 120 amino acids to about 260 amino acids, about 120 amino acids to about 240 amino acids, about 120 amino acids to about 220 amino acids, about 120 amino acids to about 200 amino acids, about 120 amino acids to about 195 amino acids, about 120 amino acids to about 190 amino acids, about 120 amino acids to about 185 amino acids, about 120 amino acids to about 180 amino acids, about 120 amino acids to about 175 amino acids, about 120 amino acids to about 170 amino acids, about 120 amino acids to about 165 amino acids, about 120 amino acids to about 160 amino acids, about 120 amino acids to about 155 amino acids, about 120 amino acids to about 150 amino acids, about 120 amino acids to about 145 amino acids, about 120 amino acids to about 140 amino acids, about 120 amino acids to about 135 amino acids, about 120 amino acids to about 130 amino acids, about 120 amino acids to about 125 amino acids, about 125 amino acids to about 1000 amino acids, about 125 amino acids to about 950 amino acids, about 125 amino acids to about 900 amino acids, about 125 amino acids to about 850 amino acids, about 125 amino acids to about 800 amino acids, about 125 amino acids to about 750 amino acids, about 125 amino acids to about 700 amino acids, about 125 amino acids to about 650 amino acids, about 125 amino acids to about 600 amino acids, about 125 amino acids to about 550 amino acids, about 125 amino acids to about 500 amino acids, about 125 amino acids to about 450 amino acids, about 125 amino acids to about 400 amino acids, about 125 amino acids to about 350 amino acids, about 125 amino acids to about 300 amino acids, about 125 amino acids to about 280 amino acids, about 125 amino acids to about 260 amino acids, about 125 amino acids to about 240 amino acids, about 125 amino acids to about 220 amino acids, about 125 amino acids to about 200 amino acids, about 125 amino acids to about 195 amino acids, about 125 amino acids to about 190 amino acids, about 125 amino acids to about 185 amino acids, about 125 amino acids to about 180 amino acids, about 125 amino acids to about 175 amino acids, about 125 amino acids to about 170 amino acids, about 125 amino acids to about 165 amino acids, about 125 amino acids to about 160 amino acids, about 125 amino acids to about 155 amino acids, about 125 amino acids to about 150 amino acids, about 125 amino acids to about 145 amino acids, about 125 amino acids to about 140 amino acids, about 125 amino acids to about 135 amino acids, about 125 amino acids to about 130 amino acids, about 130 amino acids to about 1000 amino acids, about 130 amino acids to about 950 amino acids, about 130 amino acids to about 900 amino acids, about 130 amino acids to about 850 amino acids, about 130 amino acids to about 800 amino acids, about 130 amino acids to about 750 amino acids, about 130 amino acids to about 700 amino acids, about 130 amino acids to about 650 amino acids, about 130 amino acids to about 600 amino acids, about 130 amino acids to about 550 amino acids, about 130 amino acids to about 500 amino acids, about 130 amino acids to about 450 amino acids, about 130 amino acids to about 400 amino acids, about 130 amino acids to about 350 amino acids, about 130 amino acids to about 300 amino acids, about 130 amino acids to about 280 amino acids, about 130 amino acids to about 260 amino acids, about 130 amino acids to about 240 amino acids, about 130 amino acids to about 220 amino acids, about 130 amino acids to about 200 amino acids, about 130 amino acids to about 195 amino acids, about 130 amino acids to about 190 amino acids, about 130 amino acids to about 185 amino acids, about 130 amino acids to about 180 amino acids, about 130 amino acids to about 175 amino acids, about 130 amino acids to about 170 amino acids, about 130 amino acids to about 165 amino acids, about 130 amino acids to about 160 amino acids, about 130 amino acids to about 155 amino acids, about 130 amino acids to about 150 amino acids, about 130 amino acids to about 145 amino acids, about 130 amino acids to about 140 amino acids, about 130 amino acids to about 135 amino acids, about 135 amino acids to about 1000 amino acids, about 135 amino acids to about 950 amino acids, about 135 amino acids to about 900 amino acids, about 135 amino acids to about 850 amino acids, about 135 amino acids to about 800 amino acids, about 135 amino acids to about 750 amino acids, about 135 amino acids to about 700 amino acids, about 135 amino acids to about 650 amino acids, about 135 amino acids to about 600 amino acids, about 135 amino acids to about 550 amino acids, about 135 amino acids to about 500 amino acids, about 135 amino acids to about 450 amino acids, about 135 amino acids to about 400 amino acids, about 135 amino acids to about 350 amino acids, about 135 amino acids to about 300 amino acids, about 135 amino acids to about 280 amino acids, about 135 amino acids to about 260 amino acids, about 135 amino acids to about 240 amino acids, about 135 amino acids to about 220 amino acids, about 135 amino acids to about 200 amino acids, about 135 amino acids to about 195 amino acids, about 135 amino acids to about 190 amino acids, about 135 amino acids to about 185 amino acids, about 135 amino acids to about 180 amino acids, about 135 amino acids to about 175 amino acids, about 135 amino acids to about 170 amino acids, about 135 amino acids to about 165 amino acids, about 135 amino acids to about 160 amino acids, about 135 amino acids to about 155 amino acids, about 135 amino acids to about 150 amino acids, about 135 amino acids to about 145 amino acids, about 135 amino acids to about 140 amino acids, about 140 amino acids to about 1000 amino acids, about 140 amino acids to about 950 amino acids, about 140 amino acids to about 900 amino acids, about 140 amino acids to about 850 amino acids, about 140 amino acids to about 800 amino acids, about 140 amino acids to about 750 amino acids, about 140 amino acids to about 700 amino acids, about 140 amino acids to about 650 amino acids, about 140 amino acids to about 600 amino acids, about 140 amino acids to about 550 amino acids, about 140 amino acids to about 500 amino acids, about 140 amino acids to about 450 amino acids, about 140 amino acids to about 400 amino acids, about 140 amino acids to about 350 amino acids, about 140 amino acids to about 300 amino acids, about 140 amino acids to about 280 amino acids, about 140 amino acids to about 260 amino acids, about 140 amino acids to about 240 amino acids, about 140 amino acids to about 220 amino acids, about 140 amino acids to about 200 amino acids, about 140 amino acids to about 195 amino acids, about 140 amino acids to about 190 amino acids, about 140 amino acids to about 185 amino acids, about 140 amino acids to about 180 amino acids, about 140 amino acids to about 175 amino acids, about 140 amino acids to about 170 amino acids, about 140 amino acids to about 165 amino acids, about 140 amino acids to about 160 amino acids, about 140 amino acids to about 155 amino acids, about 140 amino acids to about 150 amino acids, about 140 amino acids to about 145 amino acids, about 145 amino acids to about 1000 amino acids, about 145 amino acids to about 950 amino acids, about 145 amino acids to about 900 amino acids, about 145 amino acids to about 850 amino acids, about 145 amino acids to about 800 amino acids, about 145 amino acids to about 750 amino acids, about 145 amino acids to about 700 amino acids, about 145 amino acids to about 650 amino acids, about 145 amino acids to about 600 amino acids, about 145 amino acids to about 550 amino acids, about 145 amino acids to about 500 amino acids, about 145 amino acids to about 450 amino acids, about 145 amino acids to about 400 amino acids, about 145 amino acids to about 350 amino acids, about 145 amino acids to about 300 amino acids, about 145 amino acids to about 280 amino acids, about 145 amino acids to about 260 amino acids, about 145 amino acids to about 240 amino acids, about 145 amino acids to about 220 amino acids, about 145 amino acids to about 200 amino acids, about 145 amino acids to about 195 amino acids, about 145 amino acids to about 190 amino acids, about 145 amino acids to about 185 amino acids, about 145 amino acids to about 180 amino acids, about 145 amino acids to about 175 amino acids, about 145 amino acids to about 170 amino acids, about 145 amino acids to about 165 amino acids, about 145 amino acids to about 160 amino acids, about 145 amino acids to about 155 amino acids, about 145 amino acids to about 150 amino acids, about 150 amino acids to about 1000 amino acids, about 150 amino acids to about 950 amino acids, about 150 amino acids to about 900 amino acids, about 150 amino acids to about 850 amino acids, about 150 amino acids to about 800 amino acids, about 150 amino acids to about 750 amino acids, about 150 amino acids to about 700 amino acids, about 150 amino acids to about 650 amino acids, about 150 amino acids to about 600 amino acids, about 150 amino acids to about 550 amino acids, about 150 amino acids to about 500 amino acids, about 150 amino acids to about 450 amino acids, about 150 amino acids to about 400 amino acids, about 150 amino acids to about 350 amino acids, about 150 amino acids to about 300 amino acids, about 150 amino acids to about 280 amino acids, about 150 amino acids to about 260 amino acids, about 150 amino acids to about 240 amino acids, about 150 amino acids to about 220 amino acids, about 150 amino acids to about 200 amino acids, about 150 amino acids to about 195 amino acids, about 150 amino acids to about 190 amino acids, about 150 amino acids to about 185 amino acids, about 150 amino acids to about 180 amino acids, about 150 amino acids to about 175 amino acids, about 150 amino acids to about 170 amino acids, about 150 amino acids to about 165 amino acids, about 150 amino acids to about 160 amino acids, about 150 amino acids to about 155 amino acids, about 155 amino acids to about 1000 amino acids, about 155 amino acids to about 950 amino acids, about 155 amino acids to about 900 amino acids, about 155 amino acids to about 850 amino acids, about 155 amino acids to about 800 amino acids, about 155 amino acids to about 750 amino acids, about 155 amino acids to about 700 amino acids, about 155 amino acids to about 650 amino acids, about 155 amino acids to about 600 amino acids, about 155 amino acids to about 550 amino acids, about 155 amino acids to about 500 amino acids, about 155 amino acids to about 450 amino acids, about 155 amino acids to about 400 amino acids, about 155 amino acids to about 350 amino acids, about 155 amino acids to about 300 amino acids, about 155 amino acids to about 280 amino acids, about 155 amino acids to about 260 amino acids, about 155 amino acids to about 240 amino acids, about 155 amino acids to about 220 amino acids, about 155 amino acids to about 200 amino acids, about 155 amino acids to about 195 amino acids, about 155 amino acids to about 190 amino acids, about 155 amino acids to about 185 amino acids, about 155 amino acids to about 180 amino acids, about 155 amino acids to about 175 amino acids, about 155 amino acids to about 170 amino acids, about 155 amino acids to about 165 amino acids, about 155 amino acids to about 160 amino acids, about 160 amino acids to about 1000 amino acids, about 160 amino acids to about 950 amino acids, about 160 amino acids to about 900 amino acids, about 160 amino acids to about 850 amino acids, about 160 amino acids to about 800 amino acids, about 160 amino acids to about 750 amino acids, about 160 amino acids to about 700 amino acids, about 160 amino acids to about 650 amino acids, about 160 amino acids to about 600 amino acids, about 160 amino acids to about 550 amino acids, about 160 amino acids to about 500 amino acids, about 160 amino acids to about 450 amino acids, about 160 amino acids to about 400 amino acids, about 160 amino acids to about 350 amino acids, about 160 amino acids to about 300 amino acids, about 160 amino acids to about 280 amino acids, about 160 amino acids to about 260 amino acids, about 160 amino acids to about 240 amino acids, about 160 amino acids to about 220 amino acids, about 160 amino acids to about 200 amino acids, about 160 amino acids to about 195 amino acids, about 160 amino acids to about 190 amino acids, about 160 amino acids to about 185 amino acids, about 160 amino acids to about 180 amino acids, about 160 amino acids to about 175 amino acids, about 160 amino acids to about 170 amino acids, about 160 amino acids to about 165 amino acids, about 165 amino acids to about 1000 amino acids, about 165 amino acids to about 950 amino acids, about 165 amino acids to about 900 amino acids, about 165 amino acids to about 850 amino acids, about 165 amino acids to about 800 amino acids, about 165 amino acids to about 750 amino acids, about 165 amino acids to about 700 amino acids, about 165 amino acids to about 650 amino acids, about 165 amino acids to about 600 amino acids, about 165 amino acids to about 550 amino acids, about 165 amino acids to about 500 amino acids, about 165 amino acids to about 450 amino acids, about 165 amino acids to about 400 amino acids, about 165 amino acids to about 350 amino acids, about 165 amino acids to about 300 amino acids, about 165 amino acids to about 280 amino acids, about 165 amino acids to about 260 amino acids, about 165 amino acids to about 240 amino acids, about 165 amino acids to about 220 amino acids, about 165 amino acids to about 200 amino acids, about 165 amino acids to about 195 amino acids, about 165 amino acids to about 190 amino acids, about 165 amino acids to about 185 amino acids, about 165 amino acids to about 180 amino acids, about 165 amino acids to about 175 amino acids, about 165 amino acids to about 170 amino acids, about 170 amino acids to about 1000 amino acids, about 170 amino acids to about 950 amino acids, about 170 amino acids to about 900 amino acids, about 170 amino acids to about 850 amino acids, about 170 amino acids to about 800 amino acids, about 170 amino acids to about 750 amino acids, about 170 amino acids to about 700 amino acids, about 170 amino acids to about 650 amino acids, about 170 amino acids to about 600 amino acids, about 170 amino acids to about 550 amino acids, about 170 amino acids to about 500 amino acids, about 170 amino acids to about 450 amino acids, about 170 amino acids to about 400 amino acids, about 170 amino acids to about 350 amino acids, about 170 amino acids to about 300 amino acids, about 170 amino acids to about 280 amino acids, about 170 amino acids to about 260 amino acids, about 170 amino acids to about 240 amino acids, about 170 amino acids to about 220 amino acids, about 170 amino acids to about 200 amino acids, about 170 amino acids to about 195 amino acids, about 170 amino acids to about 190 amino acids, about 170 amino acids to about 185 amino acids, about 170 amino acids to about 180 amino acids, about 170 amino acids to about 175 amino acids, about 175 amino acids to about 1000 amino acids, about 175 amino acids to about 950 amino acids, about 175 amino acids to about 900 amino acids, about 175 amino acids to about 850 amino acids, about 175 amino acids to about 800 amino acids, about 175 amino acids to about 750 amino acids, about 175 amino acids to about 700 amino acids, about 175 amino acids to about 650 amino acids, about 175 amino acids to about 600 amino acids, about 175 amino acids to about 550 amino acids, about 175 amino acids to about 500 amino acids, about 175 amino acids to about 450 amino acids, about 175 amino acids to about 400 amino acids, about 175 amino acids to about 350 amino acids, about 175 amino acids to about 300 amino acids, about 175 amino acids to about 280 amino acids, about 175 amino acids to about 260 amino acids, about 175 amino acids to about 240 amino acids, about 175 amino acids to about 220 amino acids, about 175 amino acids to about 200 amino acids, about 175 amino acids to about 195 amino acids, about 175 amino acids to about 190 amino acids, about 175 amino acids to about 185 amino acids, about 175 amino acids to about 180 amino acids, about 180 amino acids to about 1000 amino acids, about 180 amino acids to about 950 amino acids, about 180 amino acids to about 900 amino acids, about 180 amino acids to about 850 amino acids, about 180 amino acids to about 800 amino acids, about 180 amino acids to about 750 amino acids, about 180 amino acids to about 700 amino acids, about 180 amino acids to about 650 amino acids, about 180 amino acids to about 600 amino acids, about 180 amino acids to about 550 amino acids, about 180 amino acids to about 500 amino acids, about 180 amino acids to about 450 amino acids, about 180 amino acids to about 400 amino acids, about 180 amino acids to about 350 amino acids, about 180 amino acids to about 300 amino acids, about 180 amino acids to about 280 amino acids, about 180 amino acids to about 260 amino acids, about 180 amino acids to about 240 amino acids, about 180 amino acids to about 220 amino acids, about 180 amino acids to about 200 amino acids, about 180 amino acids to about 195 amino acids, about 180 amino acids to about 190 amino acids, about 180 amino acids to about 185 amino acids, about 185 amino acids to about 1000 amino acids, about 185 amino acids to about 950 amino acids, about 185 amino acids to about 900 amino acids, about 185 amino acids to about 850 amino acids, about 185 amino acids to about 800 amino acids, about 185 amino acids to about 750 amino acids, about 185 amino acids to about 700 amino acids, about 185 amino acids to about 650 amino acids, about 185 amino acids to about 600 amino acids, about 185 amino acids to about 550 amino acids, about 185 amino acids to about 500 amino acids, about 185 amino acids to about 450 amino acids, about 185 amino acids to about 400 amino acids, about 185 amino acids to about 350 amino acids, about 185 amino acids to about 300 amino acids, about 185 amino acids to about 280 amino acids, about 185 amino acids to about 260 amino acids, about 185 amino acids to about 240 amino acids, about 185 amino acids to about 220 amino acids, about 185 amino acids to about 200 amino acids, about 185 amino acids to about 195 amino acids, about 185 amino acids to about 190 amino acids, about 190 amino acids to about 1000 amino acids, about 190 amino acids to about 950 amino acids, about 190 amino acids to about 900 amino acids, about 190 amino acids to about 850 amino acids, about 190 amino acids to about 800 amino acids, about 190 amino acids to about 750 amino acids, about 190 amino acids to about 700 amino acids, about 190 amino acids to about 650 amino acids, about 190 amino acids to about 600 amino acids, about 190 amino acids to about 550 amino acids, about 190 amino acids to about 500 amino acids, about 190 amino acids to about 450 amino acids, about 190 amino acids to about 400 amino acids, about 190 amino acids to about 350 amino acids, about 190 amino acids to about 300 amino acids, about 190 amino acids to about 280 amino acids, about 190 amino acids to about 260 amino acids, about 190 amino acids to about 240 amino acids, about 190 amino acids to about 220 amino acids, about 190 amino acids to about 200 amino acids, about 190 amino acids to about 195 amino acids, about 195 amino acids to about 1000 amino acids, about 195 amino acids to about 950 amino acids, about 195 amino acids to about 900 amino acids, about 195 amino acids to about 850 amino acids, about 195 amino acids to about 800 amino acids, about 195 amino acids to about 750 amino acids, about 195 amino acids to about 700 amino acids, about 195 amino acids to about 650 amino acids, about 195 amino acids to about 600 amino acids, about 195 amino acids to about 550 amino acids, about 195 amino acids to about 500 amino acids, about 195 amino acids to about 450 amino acids, about 195 amino acids to about 400 amino acids, about 195 amino acids to about 350 amino acids, about 195 amino acids to about 300 amino acids, about 195 amino acids to about 280 amino acids, about 195 amino acids to about 260 amino acids, about 195 amino acids to about 240 amino acids, about 195 amino acids to about 220 amino acids, about 195 amino acids to about 200 amino acids, about 200 amino acids to about 1000 amino acids, about 200 amino acids to about 950 amino acids, about 200 amino acids to about 900 amino acids, about 200 amino acids to about 850 amino acids, about 200 amino acids to about 800 amino acids, about 200 amino acids to about 750 amino acids, about 200 amino acids to about 700 amino acids, about 200 amino acids to about 650 amino acids, about 200 amino acids to about 600 amino acids, about 200 amino acids to about 550 amino acids, about 200 amino acids to about 500 amino acids, about 200 amino acids to about 450 amino acids, about 200 amino acids to about 400 amino acids, about 200 amino acids to about 350 amino acids, about 200 amino acids to about 300 amino acids, about 200 amino acids to about 280 amino acids, about 200 amino acids to about 260 amino acids, about 200 amino acids to about 240 amino acids, about 200 amino acids to about 220 amino acids, about 220 amino acids to about 1000 amino acids, about 220 amino acids to about 950 amino acids, about 220 amino acids to about 900 amino acids, about 220 amino acids to about 850 amino acids, about 220 amino acids to about 800 amino acids, about 220 amino acids to about 750 amino acids, about 220 amino acids to about 700 amino acids, about 220 amino acids to about 650 amino acids, about 220 amino acids to about 600 amino acids, about 220 amino acids to about 550 amino acids, about 220 amino acids to about 500 amino acids, about 220 amino acids to about 450 amino acids, about 220 amino acids to about 400 amino acids, about 220 amino acids to about 350 amino acids, about 220 amino acids to about 300 amino acids, about 220 amino acids to about 280 amino acids, about 220 amino acids to about 260 amino acids, about 220 amino acids to about 240 amino acids, about 240 amino acids to about 1000 amino acids, about 240 amino acids to about 950 amino acids, about 240 amino acids to about 900 amino acids, about 240 amino acids to about 850 amino acids, about 240 amino acids to about 800 amino acids, about 240 amino acids to about 750 amino acids, about 240 amino acids to about 700 amino acids, about 240 amino acids to about 650 amino acids, about 240 amino acids to about 600 amino acids, about 240 amino acids to about 550 amino acids, about 240 amino acids to about 500 amino acids, about 240 amino acids to about 450 amino acids, about 240 amino acids to about 400 amino acids, about 240 amino acids to about 350 amino acids, about 240 amino acids to about 300 amino acids, about 240 amino acids to about 280 amino acids, about 240 amino acids to about 260 amino acids, about 260 amino acids to about 1000 amino acids, about 260 amino acids to about 950 amino acids, about 260 amino acids to about 900 amino acids, about 260 amino acids to about 850 amino acids, about 260 amino acids to about 800 amino acids, about 260 amino acids to about 750 amino acids, about 260 amino acids to about 700 amino acids, about 260 amino acids to about 650 amino acids, about 260 amino acids to about 600 amino acids, about 260 amino acids to about 550 amino acids, about 260 amino acids to about 500 amino acids, about 260 amino acids to about 450 amino acids, about 260 amino acids to about 400 amino acids, about 260 amino acids to about 350 amino acids, about 260 amino acids to about 300 amino acids, about 260 amino acids to about 280 amino acids, about 280 amino acids to about 1000 amino acids, about 280 amino acids to about 950 amino acids, about 280 amino acids to about 900 amino acids, about 280 amino acids to about 850 amino acids, about 280 amino acids to about 800 amino acids, about 280 amino acids to about 750 amino acids, about 280 amino acids to about 700 amino acids, about 280 amino acids to about 650 amino acids, about 280 amino acids to about 600 amino acids, about 280 amino acids to about 550 amino acids, about 280 amino acids to about 500 amino acids, about 280 amino acids to about 450 amino acids, about 280 amino acids to about 400 amino acids, about 280 amino acids to about 350 amino acids, about 280 amino acids to about 300 amino acids, about 300 amino acids to about 1000 amino acids, about 300 amino acids to about 950 amino acids, about 300 amino acids to about 900 amino acids, about 300 amino acids to about 850 amino acids, about 300 amino acids to about 800 amino acids, about 300 amino acids to about 750 amino acids, about 300 amino acids to about 700 amino acids, about 300 amino acids to about 650 amino acids, about 300 amino acids to about 600 amino acids, about 300 amino acids to about 550 amino acids, about 300 amino acids to about 500 amino acids, about 300 amino acids to about 450 amino acids, about 300 amino acids to about 400 amino acids, about 300 amino acids to about 350 amino acids, about 350 amino acids to about 1000 amino acids, about 350 amino acids to about 950 amino acids, about 350 amino acids to about 900 amino acids, about 350 amino acids to about 850 amino acids, about 350 amino acids to about 800 amino acids, about 350 amino acids to about 750 amino acids, about 350 amino acids to about 700 amino acids, about 350 amino acids to about 650 amino acids, about 350 amino acids to about 600 amino acids, about 350 amino acids to about 550 amino acids, about 350 amino acids to about 500 amino acids, about 350 amino acids to about 450 amino acids, about 350 amino acids to about 400 amino acids, about 400 amino acids to about 1000 amino acids, about 400 amino acids to about 950 amino acids, about 400 amino acids to about 900 amino acids, about 400 amino acids to about 850 amino acids, about 400 amino acids to about 800 amino acids, about 400 amino acids to about 750 amino acids, about 400 amino acids to about 700 amino acids, about 400 amino acids to about 650 amino acids, about 400 amino acids to about 600 amino acids, about 400 amino acids to about 550 amino acids, about 400 amino acids to about 500 amino acids, about 400 amino acids to about 450 amino acids, about 450 amino acids to about 1000 amino acids, about 450 amino acids to about 950 amino acids, about 450 amino acids to about 900 amino acids, about 450 amino acids to about 850 amino acids, about 450 amino acids to about 800 amino acids, about 450 amino acids to about 750 amino acids, about 450 amino acids to about 700 amino acids, about 450 amino acids to about 650 amino acids, about 450 amino acids to about 600 amino acids, about 450 amino acids to about 550 amino acids, about 450 amino acids to about 500 amino acids, about 500 amino acids to about 1000 amino acids, about 500 amino acids to about 950 amino acids, about 500 amino acids to about 900 amino acids, about 500 amino acids to about 850 amino acids, about 500 amino acids to about 800 amino acids, about 500 amino acids to about 750 amino acids, about 500 amino acids to about 700 amino acids, about 500 amino acids to about 650 amino acids, about 500 amino acids to about 600 amino acids, about 500 amino acids to about 550 amino acids, about 550 amino acids to about 1000 amino acids, about 550 amino acids to about 950 amino acids, about 550 amino acids to about 900 amino acids, about 550 amino acids to about 850 amino acids, about 550 amino acids to about 800 amino acids, about 550 amino acids to about 750 amino acids, about 550 amino acids to about 700 amino acids, about 550 amino acids to about 650 amino acids, about 550 amino acids to about 600 amino acids, about 600 amino acids to about 1000 amino acids, about 600 amino acids to about 950 amino acids, about 600 amino acids to about 900 amino acids, about 600 amino acids to about 850 amino acids, about 600 amino acids to about 800 amino acids, about 600 amino acids to about 750 amino acids, about 600 amino acids to about 700 amino acids, about 600 amino acids to about 650 amino acids, about 650 amino acids to about 1000 amino acids, about 650 amino acids to about 950 amino acids, about 650 amino acids to about 900 amino acids, about 650 amino acids to about 850 amino acids, about 650 amino acids to about 800 amino acids, about 650 amino acids to about 750 amino acids, about 650 amino acids to about 700 amino acids, about 700 amino acids to about 1000 amino acids, about 700 amino acids to about 950 amino acids, about 700 amino acids to about 900 amino acids, about 700 amino acids to about 850 amino acids, about 700 amino acids to about 800 amino acids, about 700 amino acids to about 750 amino acids, about 750 amino acids to about 1000 amino acids, about 750 amino acids to about 950 amino acids, about 750 amino acids to about 900 amino acids, about 750 amino acids to about 850 amino acids, about 750 amino acids to about 800 amino acids, about 800 amino acids to about 1000 amino acids, about 800 amino acids to about 950 amino acids, about 800 amino acids to about 900 amino acids, about 800 amino acids to about 850 amino acids, about 850 amino acids to about 1000 amino acids, about 850 amino acids to about 950 amino acids, about 850 amino acids to about 900 amino acids, about 900 amino acids to about 1000 amino acids, about 900 amino acids to about 950 amino acids, or about 950 amino acids to about 1000 amino acids.

Any of the target-binding domains described herein can bind to a ligand of TGF-βRII with a dissociation equilibrium constant (K_{D}) of less than 1×10⁻⁷ M, less than 1×10⁻⁸M, less than 1×10⁻⁹ M, less than 1×10⁻¹⁰ NI less than 1×10⁻¹¹M, less than 1×10⁻¹² M, or less than 1×10⁻¹³ M. In some embodiments, the antigen-binding protein construct provided herein can bind to an identifying antigen with a K_{D} of about 1×10⁻³ M to about 1×10⁻⁵ M, about 1×10⁻⁴M to about 1×10⁻⁶M, about 1−10⁻⁵ M to about 1−10⁻⁷ M, about 1×10⁻⁶ M to about 1×10⁻⁸ M, about 1×10⁻⁷ M to about 1×10⁻⁹ M, about 1−10⁻⁸ M to about 1−10⁻¹⁰ M, or about 1×10⁻⁹ M to about 1−10⁻¹¹ M (inclusive).

Any of the target-binding domains described herein can bind to a ligand of TGF-βRII (e.g., TGF-β) with a K_{D} of between about 1 pM to about 30 nM (e.g., about 1 pM to about 25 nM, about 1 pM to about 20 nM, about 1 pM to about 15 nM, about 1 pM to about 10 nM, about 1 pM to about 5 nM, about 1 pM to about 2 nM, about 1 pM to about 1 nM, about 1 pM to about 950 pM, about 1 pM to about 900 pM, about 1 pM to about 850 pM, about 1 pM to about 800 pM, about 1 pM to about 750 pM, about 1 pM to about 700 pM, about 1 pM to about 650 pM, about 1 pM to about 600 pM, about 1 pM to about 550 pM, about 1 pM to about 500 pM, about 1 pM to about 450 pM, about 1 pM to about 400 pM, about 1 pM to about 350 pM, about 1 pM to about 300 pM, about 1 pM to about 250 pM, about 1 pM to about 200 pM, about 1 pM to about 150 pM, about 1 pM to about 100 pM, about 1 pM to about 90 pM, about 1 pM to about 80 pM, about 1 pM to about 70 pM, about 1 pM to about 60 pM, about 1 pM to about 50 pM, about 1 pM to about 40 pM, about 1 pM to about 30 pM, about 1 pM to about 20 pM, about 1 pM to about 10 pM, about 1 pM to about 5 pM, about 1 pM to about 4 pM, about 1 pM to about 3 pM, about 1 pM to about 2 pM, about 2 pM to about 30 nM, about 2 pM to about 25 nM, about 2 pM to about 20 nM, about 2 pM to about 15 nM, about 2 pM to about 10 nM, about 2 pM to about 5 nM, about 2 pM to about 2 nM, about 2 pM to about 1 nM, about 2 pM to about 950 pM, about 2 pM to about 900 pM, about 2 pM to about 850 pM, about 2 pM to about 800 pM, about 2 pM to about 750 pM, about 2 pM to about 700 pM, about 2 pM to about 650 pM, about 2 pM to about 600 pM, about 2 pM to about 550 pM, about 2 pM to about 500 pM, about 2 pM to about 450 pM, about 2 pM to about 400 pM, about 2 pM to about 350 pM, about 2 pM to about 300 pM, about 2 pM to about 250 pM, about 2 pM to about 200 pM, about 2 pM to about 150 pM, about 2 pM to about 100 pM, about 2 pM to about 90 pM, about 2 pM to about 80 pM, about 2 pM to about 70 pM, about 2 pM to about 60 pM, about 2 pM to about 50 pM, about 2 pM to about 40 pM, about 2 pM to about 30 pM, about 2 pM to about 20 pM, about 2 pM to about 10 pM, about 2 pM to about 5 pM, about 2 pM to about 4 pM, about 2 pM to about 3 pM, about 5 pM to about 30 nM, about 5 pM to about 25 nM, about 5 pM to about 20 nM, about 5 pM to about 15 nM, about 5 pM to about 10 nM, about 5 pM to about 5 nM, about 5 pM to about 2 nM, about 5 pM to about 1 nM, about 5 pM to about 950 pM, about 5 pM to about 900 pM, about 5 pM to about 850 pM, about 5 pM to about 800 pM, about 5 pM to about 750 pM, about 5 pM to about 700 pM, about 5 pM to about 650 pM, about 5 pM to about 600 pM, about 5 pM to about 550 pM, about 5 pM to about 500 pM, about 5 pM to about 450 pM, about 5 pM to about 400 pM, about 5 pM to about 350 pM, about 5 pM to about 300 pM, about 5 pM to about 250 pM, about 5 pM to about 200 pM, about 5 pM to about 150 pM, about 5 pM to about 100 pM, about 5 pM to about 90 pM, about 5 pM to about 80 pM, about 5 pM to about 70 pM, about 5 pM to about 60 pM, about 5 pM to about 50 pM, about 5 pM to about 40 pM, about 5 pM to about 30 pM, about 5 pM to about 20 pM, about 5 pM to about 10 pM, about 10 pM to about 30 nM, about 10 pM to about 25 nM, about 10 pM to about 20 nM, about 10 pM to about 15 nM, about 10 pM to about 10 nM, about 10 pM to about 5 nM, about 10 pM to about 2 nM, about 10 pM to about 1 nM, about 10 pM to about 950 pM, about 10 pM to about 900 pM, about 10 pM to about 850 pM, about 10 pM to about 800 pM, about 10 pM to about 750 pM, about 10 pM to about 700 pM, about 10 pM to about 650 pM, about 10 pM to about 600 pM, about 10 pM to about 550 pM, about 10 pM to about 500 pM, about 10 pM to about 450 pM, about 10 pM to about 400 pM, about 10 pM to about 350 pM, about 10 pM to about 300 pM, about 10 pM to about 250 pM, about 10 pM to about 200 pM, about 10 pM to about 150 pM, about 10 pM to about 100 pM, about 10 pM to about 90 pM, about 10 pM to about 80 pM, about 10 pM to about 70 pM, about 10 pM to about 60 pM, about 10 pM to about 50 pM, about 10 pM to about 40 pM, about 10 pM to about 30 pM, about 10 pM to about 20 pM, about 15 pM to about 30 nM, about 15 pM to about 25 nM, about 15 pM to about 20 nM, about 15 pM to about 15 nM, about 15 pM to about 10 nM, about 15 pM to about 5 nM, about 15 pM to about 2 nM, about 15 pM to about 1 nM, about 15 pM to about 950 pM, about 15 pM to about 900 pM, about 15 pM to about 850 pM, about 15 pM to about 800 pM, about 15 pM to about 750 pM, about 15 pM to about 700 pM, about 15 pM to about 650 pM, about 15 pM to about 600 pM, about 15 pM to about 550 pM, about 15 pM to about 500 pM, about 15 pM to about 450 pM, about 15 pM to about 400 pM, about 15 pM to about 350 pM, about 15 pM to about 300 pM, about 15 pM to about 250 pM, about 15 pM to about 200 pM, about 15 pM to about 150 pM, about 15 pM to about 100 pM, about 15 pM to about 90 pM, about 15 pM to about 80 pM, about 15 pM to about 70 pM, about 15 pM to about 60 pM, about 15 pM to about 50 pM, about 15 pM to about 40 pM, about 15 pM to about 30 pM, about 15 pM to about 20 pM, about 20 pM to about 30 nM, about 20 pM to about 25 nM, about 20 pM to about 20 nM, about 20 pM to about 15 nM, about 20 pM to about 10 nM, about 20 pM to about 5 nM, about 20 pM to about 2 nM, about 20 pM to about 1 nM, about 20 pM to about 950 pM, about 20 pM to about 900 pM, about 20 pM to about 850 pM, about 20 pM to about 800 pM, about 20 pM to about 750 pM, about 20 pM to about 700 pM, about 20 pM to about 650 pM, about 20 pM to about 600 pM, about 20 pM to about 550 pM, about 20 pM to about 500 pM, about 20 pM to about 450 pM, about 20 pM to about 400 pM, about 20 pM to about 350 pM, about 20 pM to about 300 pM, about 20 pM to about 250 pM, about 20 pM to about 20 pM, about 200 pM to about 150 pM, about 20 pM to about 100 pM, about 20 pM to about 90 pM, about 20 pM to about 80 pM, about 20 pM to about 70 pM, about 20 pM to about 60 pM, about 20 pM to about 50 pM, about 20 pM to about 40 pM, about 20 pM to about 30 pM, about 30 pM to about 30 nM, about 30 pM to about 25 nM, about 30 pM to about 30 nM, about 30 pM to about 15 nM, about 30 pM to about 10 nM, about 30 pM to about 5 nM, about 30 pM to about 2 nM, about 30 pM to about 1 nM, about 30 pM to about 950 pM, about 30 pM to about 900 pM, about 30 pM to about 850 pM, about 30 pM to about 800 pM, about 30 pM to about 750 pM, about 30 pM to about 700 pM, about 30 pM to about 650 pM, about 30 pM to about 600 pM, about 30 pM to about 550 pM, about 30 pM to about 500 pM, about 30 pM to about 450 pM, about 30 pM to about 400 pM, about 30 pM to about 350 pM, about 30 pM to about 300 pM, about 30 pM to about 250 pM, about 30 pM to about 200 pM, about 30 pM to about 150 pM, about 30 pM to about 100 pM, about 30 pM to about 90 pM, about 30 pM to about 80 pM, about 30 pM to about 70 pM, about 30 pM to about 60 pM, about 30 pM to about 50 pM, about 30 pM to about 40 pM, about 40 pM to about 30 nM, about 40 pM to about 25 nM, about 40 pM to about 30 nM, about 40 pM to about 15 nM, about 40 pM to about 10 nM, about 40 pM to about 5 nM, about 40 pM to about 2 nM, about 40 pM to about 1 nM, about 40 pM to about 950 pM, about 40 pM to about 900 pM, about 40 pM to about 850 pM, about 40 pM to about 800 pM, about 40 pM to about 750 pM, about 40 pM to about 700 pM, about 40 pM to about 650 pM, about 40 pM to about 600 pM, about 40 pM to about 550 pM, about 40 pM to about 500 pM, about 40 pM to about 450 pM, about 40 pM to about 400 pM, about 40 pM to about 350 pM, about 40 pM to about 300 pM, about 40 pM to about 250 pM, about 40 pM to about 200 pM, about 40 pM to about 150 pM, about 40 pM to about 100 pM, about 40 pM to about 90 pM, about 40 pM to about 80 pM, about 40 pM to about 70 pM, about 40 pM to about 60 pM, about 40 pM to about 50 pM, about 50 pM to about 30 nM, about 50 pM to about 25 nM, about 50 pM to about 30 nM, about 50 pM to about 15 nM, about 50 pM to about 10 nM, about 50 pM to about 5 nM, about 50 pM to about 2 nM, about 50 pM to about 1 nM, about 50 pM to about 950 pM, about 50 pM to about 900 pM, about 50 pM to about 850 pM, about 50 pM to about 800 pM, about 50 pM to about 750 pM, about 50 pM to about 700 pM, about 50 pM to about 650 pM, about 50 pM to about 600 pM, about 50 pM to about 550 pM, about 50 pM to about 500 pM, about 50 pM to about 450 pM, about 50 pM to about 400 pM, about 50 pM to about 350 pM, about 50 pM to about 300 pM, about 50 pM to about 250 pM, about 50 pM to about 200 pM, about 50 pM to about 150 pM, about 50 pM to about 100 pM, about 50 pM to about 90 pM, about 50 pM to about 80 pM, about 50 pM to about 70 pM, about 50 pM to about 60 pM, about 60 pM to about 30 nM, about 60 pM to about 25 nM, about 60 pM to about 30 nM, about 60 pM to about 15 nM, about 60 pM to about 10 nM, about 60 pM to about 5 nM, about 60 pM to about 2 nM, about 60 pM to about 1 nM, about 60 pM to about 950 pM, about 60 pM to about 900 pM, about 60 pM to about 850 pM, about 60 pM to about 800 pM, about 60 pM to about 750 pM, about 60 pM to about 700 pM, about 60 pM to about 650 pM, about 60 pM to about 600 pM, about 60 pM to about 550 pM, about 60 pM to about 500 pM, about 60 pM to about 450 pM, about 60 pM to about 400 pM, about 60 pM to about 350 pM, about 60 pM to about 300 pM, about 60 pM to about 250 pM, about 60 pM to about 200 pM, about 60 pM to about 150 pM, about 60 pM to about 100 pM, about 60 pM to about 90 pM, about 60 pM to about 80 pM, about 60 pM to about 70 pM, about 70 pM to about 30 nM, about 70 pM to about 25 nM, about 70 pM to about 30 nM, about 70 pM to about 15 nM, about 70 pM to about 10 nM, about 70 pM to about 5 nM, about 70 pM to about 2 nM, about 70 pM to about 1 nM, about 70 pM to about 950 pM, about 70 pM to about 900 pM, about 70 pM to about 850 pM, about 70 pM to about 800 pM, about 70 pM to about 750 pM, about 70 pM to about 700 pM, about 70 pM to about 650 pM, about 70 pM to about 600 pM, about 70 pM to about 550 pM, about 70 pM to about 500 pM, about 70 pM to about 450 pM, about 70 pM to about 400 pM, about 70 pM to about 350 pM, about 70 pM to about 300 pM, about 70 pM to about 250 pM, about 70 pM to about 200 pM, about 70 pM to about 150 pM, about 70 pM to about 100 pM, about 70 pM to about 90 pM, about 70 pM to about 80 pM, about 80 pM to about 30 nM, about 80 pM to about 25 nM, about 80 pM to about 30 nM, about 80 pM to about 15 nM, about 80 pM to about 10 nM, about 80 pM to about 5 nM, about 80 pM to about 2 nM, about 80 pM to about 1 nM, about 80 pM to about 950 pM, about 80 pM to about 900 pM, about 80 pM to about 850 pM, about 80 pM to about 800 pM, about 80 pM to about 750 pM, about 80 pM to about 700 pM, about 80 pM to about 650 pM, about 80 pM to about 600 pM, about 80 pM to about 550 pM, about 80 pM to about 500 pM, about 80 pM to about 450 pM, about 80 pM to about 400 pM, about 80 pM to about 350 pM, about 80 pM to about 300 pM, about 80 pM to about 250 pM, about 80 pM to about 200 pM, about 80 pM to about 150 pM, about 80 pM to about 100 pM, about 80 pM to about 90 pM, about 90 pM to about 30 nM, about 90 pM to about 25 nM, about 90 pM to about 30 nM, about 90 pM to about 15 nM, about 90 pM to about 10 nM, about 90 pM to about 5 nM, about 90 pM to about 2 nM, about 90 pM to about 1 nM, about 90 pM to about 950 pM, about 90 pM to about 900 pM, about 90 pM to about 850 pM, about 90 pM to about 800 pM, about 90 pM to about 750 pM, about 90 pM to about 700 pM, about 90 pM to about 650 pM, about 90 pM to about 600 pM, about 90 pM to about 550 pM, about 90 pM to about 500 pM, about 90 pM to about 450 pM, about 90 pM to about 400 pM, about 90 pM to about 350 pM, about 90 pM to about 300 pM, about 90 pM to about 250 pM, about 90 pM to about 200 pM, about 90 pM to about 150 pM, about 90 pM to about 100 pM, about 100 pM to about 30 nM, about 100 pM to about 25 nM, about 100 pM to about 30 nM, about 100 pM to about 15 nM, about 100 pM to about 10 nM, about 100 pM to about 5 nM, about 100 pM to about 2 nM, about 100 pM to about 1 nM, about 100 pM to about 950 pM, about 100 pM to about 900 pM, about 100 pM to about 850 pM, about 100 pM to about 800 pM, about 100 pM to about 750 pM, about 100 pM to about 700 pM, about 100 pM to about 650 pM, about 100 pM to about 600 pM, about 100 pM to about 550 pM, about 100 pM to about 500 pM, about 100 pM to about 450 pM, about 100 pM to about 400 pM, about 100 pM to about 350 pM, about 100 pM to about 300 pM, about 100 pM to about 250 pM, about 100 pM to about 200 pM, about 100 pM to about 150 pM, about 150 pM to about 30 nM, about 150 pM to about 25 nM, about 150 pM to about 30 nM, about 150 pM to about 15 nM, about 150 pM to about 10 nM, about 150 pM to about 5 nM, about 150 pM to about 2 nM, about 150 pM to about 1 nM, about 150 pM to about 950 pM, about 150 pM to about 900 pM, about 150 pM to about 850 pM, about 150 pM to about 800 pM, about 150 pM to about 750 pM, about 150 pM to about 700 pM, about 150 pM to about 650 pM, about 150 pM to about 600 pM, about 150 pM to about 550 pM, about 150 pM to about 500 pM, about 150 pM to about 450 pM, about 150 pM to about 400 pM, about 150 pM to about 350 pM, about 150 pM to about 300 pM, about 150 pM to about 250 pM, about 150 pM to about 200 pM, about 200 pM to about 30 nM, about 200 pM to about 25 nM, about 200 pM to about 30 nM, about 200 pM to about 15 nM, about 200 pM to about 10 nM, about 200 pM to about 5 nM, about 200 pM to about 2 nM, about 200 pM to about 1 nM, about 200 pM to about 950 pM, about 200 pM to about 900 pM, about 200 pM to about 850 pM, about 200 pM to about 800 pM, about 200 pM to about 750 pM, about 200 pM to about 700 pM, about 200 pM to about 650 pM, about 200 pM to about 600 pM, about 200 pM to about 550 pM, about 200 pM to about 500 pM, about 200 pM to about 450 pM, about 200 pM to about 400 pM, about 200 pM to about 350 pM, about 200 pM to about 300 pM, about 200 pM to about 250 pM, about 300 pM to about 30 nM, about 300 pM to about 25 nM, about 300 pM to about 30 nM, about 300 pM to about 15 nM, about 300 pM to about 10 nM, about 300 pM to about 5 nM, about 300 pM to about 2 nM, about 300 pM to about 1 nM, about 300 pM to about 950 pM, about 300 pM to about 900 pM, about 300 pM to about 850 pM, about 300 pM to about 800 pM, about 300 pM to about 750 pM, about 300 pM to about 700 pM, about 300 pM to about 650 pM, about 300 pM to about 600 pM, about 300 pM to about 550 pM, about 300 pM to about 500 pM, about 300 pM to about 450 pM, about 300 pM to about 400 pM, about 300 pM to about 350 pM, about 400 pM to about 30 nM, about 400 pM to about 25 nM, about 400 pM to about 30 nM, about 400 pM to about 15 nM, about 400 pM to about 10 nM, about 400 pM to about 5 nM, about 400 pM to about 2 nM, about 400 pM to about 1 nM, about 400 pM to about 950 pM, about 400 pM to about 900 pM, about 400 pM to about 850 pM, about 400 pM to about 800 pM, about 400 pM to about 750 pM, about 400 pM to about 700 pM, about 400 pM to about 650 pM, about 400 pM to about 600 pM, about 400 pM to about 550 pM, about 400 pM to about 500 pM, about 500 pM to about 30 nM, about 500 pM to about 25 nM, about 500 pM to about 30 nM, about 500 pM to about 15 nM, about 500 pM to about 10 nM, about 500 pM to about 5 nM, about 500 pM to about 2 nM, about 500 pM to about 1 nM, about 500 pM to about 950 pM, about 500 pM to about 900 pM, about 500 pM to about 850 pM, about 500 pM to about 800 pM, about 500 pM to about 750 pM, about 500 pM to about 700 pM, about 500 pM to about 650 pM, about 500 pM to about 600 pM, about 500 pM to about 550 pM, about 600 pM to about 30 nM, about 600 pM to about 25 nM, about 600 pM to about 30 nM, about 600 pM to about 15 nM, about 600 pM to about 10 nM, about 600 pM to about 5 nM, about 600 pM to about 2 nM, about 600 pM to about 1 nM, about 600 pM to about 950 pM, about 600 pM to about 900 pM, about 600 pM to about 850 pM, about 600 pM to about 800 pM, about 600 pM to about 750 pM, about 600 pM to about 700 pM, about 600 pM to about 650 pM, about 700 pM to about 30 nM, about 700 pM to about 25 nM, about 700 pM to about 30 nM, about 700 pM to about 15 nM, about 700 pM to about 10 nM, about 700 pM to about 5 nM, about 700 pM to about 2 nM, about 700 pM to about 1 nM, about 700 pM to about 950 pM, about 700 pM to about 900 pM, about 700 pM to about 850 pM, about 700 pM to about 800 pM, about 700 pM to about 750 pM, about 800 pM to about 30 nM, about 800 pM to about 25 nM, about 800 pM to about 30 nM, about 800 pM to about 15 nM, about 800 pM to about 10 nM, about 800 pM to about 5 nM, about 800 pM to about 2 nM, about 800 pM to about 1 nM, about 800 pM to about 950 pM, about 800 pM to about 900 pM, about 800 pM to about 850 pM, about 900 pM to about 30 nM, about 900 pM to about 25 nM, about 900 pM to about 30 nM, about 900 pM to about 15 nM, about 900 pM to about 10 nM, about 900 pM to about 5 nM, about 900 pM to about 2 nM, about 900 pM to about 1 nM, about 900 pM to about 950 pM, about 1 nM to about 30 nM, about 1 nM to about 25 nM, about 1 nM to about 20 nM, about 1 nM to about 15 nM, about 1 nM to about 10 nM, about 1 nM to about 5 nM, about 2 nM to about 30 nM, about 2 nM to about 25 nM, about 2 nM to about 20 nM, about 2 nM to about 15 nM, about 2 nM to about 10 nM, about 2 nM to about 5 nM, about 4 nM to about 30 nM, about 4 nM to about 25 nM, about 4 nM to about 20 nM, about 4 nM to about 15 nM, about 4 nM to about 10 nM, about 4 nM to about 5 nM, about 5 nM to about 30 nM, about 5 nM to about 25 nM, about 5 nM to about 20 nM, about 5 nM to about 15 nM, about 5 nM to about 10 nM, about 10 nM to about 30 nM, about 10 nM to about 25 nM, about 10 nM to about 20 nM, about 10 nM to about 15 nM, about 15 nM to about 30 nM, about 15 nM to about 25 nM, about 15 nM to about 20 nM, about 20 nM to about 30 nM, and about 20 nM to about 25 nM).

Any of the target-binding domains described herein can bind to a ligand of TGFβRII with a K_{D} of between about 1 nM to about 10 nM (e.g., about 1 nM to about 9 nM, about 1 nM to about 8 nM, about 1 nM to about 7 nM, about 1 nM to about 6 nM, about 1 nM to about 5 nM, about 1 nM to about 4 nM, about 1 nM to about 3 nM, about 1 nM to about 2 nM, about 2 nM to about 10 nM, about 2 nM to about 9 nM, about 2 nM to about 8 nM, about 2 nM to about 7 nM, about 2 nM to about 6 nM, about 2 nM to about 5 nM, about 2 nM to about 4 nM, about 2 nM to about 3 nM, about 3 nM to about 10 nM, about 3 nM to about 9 nM, about 3 nM to about 8 nM, about 3 nM to about 7 nM, about 3 nM to about 6 nM, about 3 nM to about 5 nM, about 3 nM to about 4 nM, about 4 nM to about 10 nM, about 4 nM to about 9 nM, about 4 nM to about 8 nM, about 4 nM to about 7 nM, about 4 nM to about 6 nM, about 4 nM to about 5 nM, about 5 nM to about 10 nM, about 5 nM to about 9 nM, about 5 nM to about 8 nM, about 5 nM to about 7 nM, about 5 nM to about 6 nM, about 6 nM to about 10 nM, about 6 nM to about 9 nM, about 6 nM to about 8 nM, about 6 nM to about 7 nM, about 7 nM to about 10 nM, about 7 nM to about 9 nM, about 7 nM to about 8 nM, about 8 nM to about 10 nM, about 8 nM to about 9 nM, and about 9 nM to about 10 nM).

A variety of different methods known in the art can be used to determine the K_{D} values of any of the antigen-binding protein constructs described herein (e.g., an electrophoretic mobility shift assay, a filter binding assay, surface plasmon resonance, and a biomolecular binding kinetics assay, etc.).

### Antigen-Binding Domains

In some embodiments of any of the multi-chain chimeric polypeptides described herein, the first target-binding domain and the second target-binding domain bind specifically to the same antigen. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of any of the multi-chain chimeric polypeptides described herein, the first target-binding domain and the second target-binding domain bind specifically to different antigens.

In some embodiments of any of the multi-chain chimeric polypeptides described herein, one or both of the first target-binding domain and the second target-binding domain is an antigen-binding domain. In some embodiments of any of the multi-chain chimeric polypeptides described herein, the first target-binding domain and the second target-binding domain are each antigen-binding domains.

In some embodiments of any of the multi-chain chimeric polypeptides described herein, the antigen-binding domain includes or is a scFv or a single domain antibody (e.g., a VHH or a VNAR domain).

In some examples, an antigen-binding domain (e.g., any of the antigen-binding domains described herein) can bind specifically to a ligand of TGF-βRII (see, e.g., antigen-binding domains that can bind specifically to TGF-β described in US 2021/0061897, US 2020/0399358, US 2020/0392221, US 2019/0315850, and US 2019/0177406, each of which is herein incorporated by reference).

The antigen-binding domains present in any of the multi-chain chimeric polypeptides described herein are each independently selected from the group consisting of: a VHH domain, a VNAR domain, and a scFv. In some embodiments, any of the antigen-binding domains described herein is a BiTe, a (scFv)2, a nanobody, a nanobody-HSA, a DART, a TandAb, a scDiabody, a scDiabody-CH3, scFv-CH-CL-scFv, a HSAbody, scDiabody-HAS, or a tandem-scFv. Additional examples of antigen-binding domains that can be used in any of the multi-chain chimeric polypeptide are known in the art.

A VHH domain is a single monomeric variable antibody domain that can be found in camelids. A VNAR domain is a single monomeric variable antibody domain that can be found in cartilaginous fish. Non-limiting aspects of VHH domains and VNAR domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments, each of the antigen-binding domains in the multi-chain chimeric polypeptides described herein are both VHH domains, or at least one antigen-binding domain is a VHH domain. In some embodiments, each of the antigen-binding domains in the multi-chain chimeric polypeptides described herein are both VNAR domains, or at least one antigen-binding domain is a VNAR domain. In some embodiments, each of the antigen-binding domains in the multi-chain chimeric polypeptides described herein are both scFv domains, or at least one antigen-binding domain is a scFv domain.

In some embodiments, two or more of polypeptides present in the multi-chain chimeric polypeptide can assemble (e.g., non-covalently assemble) to form any of the antigen-binding domains described herein, e.g., an antigen-binding fragment of an antibody (e.g., any of the antigen-binding fragments of an antibody described herein), a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, and a scFv1-PEG-scFv₂. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)₂ fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

An “Fv” fragment includes a non-covalently-linked dimer of one heavy chain variable domain and one light chain variable domain.

A “Fab” fragment includes, the constant domain of the light chain and the first constant domain (C_{H1}) of the heavy chain, in addition to the heavy and light chain variable domains of the Fv fragment.

A “F(ab′)₂” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

In some embodiments of any of the antigen-binding domains described herein can bind to an antigen selected from the group consisting of: a protein, a carbohydrate, a lipid, and a combination thereof.

Additional examples and aspects of antigen-binding domains are known in the art.

### Soluble Receptor

In some embodiments of any of the multi-chain chimeric polypeptides described herein, one or both of the first target-binding domain and the second target-binding domain is a soluble interleukin receptor, a soluble cytokine receptor or a ligand receptor. In some embodiments, the soluble receptor is a soluble TGF-β receptor II (TGF-β RII) (see, e.g., those described in Yung et al., Am. J. Resp. Crit. Care Med. 194(9):1140-1151, 2016) or a soluble TGF-βRIII (see, e.g., those described in Heng et al., Placenta 57:320,

Additional examples of soluble interleukin receptors and soluble cytokine receptors are known in the art.

### Additional Target-Binding Domains

In some embodiments of any of the multi-chain chimeric polypeptides, the first chimeric polypeptide further includes one or more (e.g., two, three, four, five, six, seven, eight, nine, or ten) additional target-binding domain(s) (e.g., any of the exemplary target-binding domains described herein or known in the art), where at least one of the one or more additional antigen-binding domain(s) is positioned between the soluble tissue factor domain (e.g., any of the exemplary soluble tissue factor domains described herein or known in the art) and the first domain of the pair of affinity domains (e.g., any of the exemplary first domains of any of the exemplary pairs of affinity domains described herein). In some embodiments, the first chimeric polypeptide can further include a linker sequence (e.g., any of the exemplary linker sequences described herein or known in the art) between the soluble tissue factor domain (e.g., any of the exemplary soluble tissue factor domains described herein) and the at least one of the one or more additional target-binding domain(s) (e.g., any of the exemplary target-binding domains described herein or known in the art), and/or a linker sequence (e.g., any of the exemplary linker sequences described herein or known in the art) between the at least one of the one or more additional target-binding domain(s) (e.g., any of the exemplary target-binding domains described herein or known in the art) and the first domain of the pair of affinity domains (e.g., any of the exemplary first domains described herein of any of the exemplary pairs of affinity domains described herein).

In some embodiments of any of the multi-chain chimeric polypeptides described herein, the first chimeric polypeptide further includes one or more (e.g., two, three, four, five, six, seven, eight, nine, or ten) additional target-binding domains at the N-terminal and/or C-terminal end of the first chimeric polypeptide. In some embodiments, at least one of the one or more additional target-binding domains (e.g., any of the exemplary target-binding domains described herein or known in the art) directly abuts the first domain of the pair of affinity domains (e.g., any of the exemplary first domains described herein of any of the exemplary pairs of affinity domains described herein) in the first chimeric polypeptide. In some embodiments, the first chimeric polypeptide further includes a linker sequence (e.g., any of the exemplary linker sequences described herein or known in the art) between the at least one of the one or more additional target-binding domains (e.g., any of the exemplary target-binding domains described herein or known in the art) and the first domain of the pair of affinity domains (e.g., any of the exemplary first domains described herein of any of the exemplary pairs of affinity domains described herein). In some embodiments, the at least one of the one or more additional target-binding domains (e.g., any of the exemplary target-binding domains described herein or known in the art) directly abuts the first target-binding domain (e.g., any of the exemplary target-binding domains described herein or known in the art) in the first chimeric polypeptide. In some embodiments, the first chimeric polypeptide further comprises a linker sequence (e.g., any of the exemplary linker sequences described herein or known in the art) between the at least one of the one or more additional target-binding domains (e.g., any of the exemplary target-binding domains described herein or known in the art) and the first target-binding domain (e.g., any of the exemplary target-binding domains described herein or known in the art).

In some embodiments of any of the multi-chain chimeric polypeptides described herein, at least one of the one or more additional target-binding domains (e.g., any of the exemplary target-binding domains described herein or known in the art) is disposed at the N- and/or C-terminus of the first chimeric polypeptide, and at least one of the one or more additional target-binding domains (e.g., any of the exemplary target-binding domains described herein or known in the art) is positioned between the soluble tissue factor domain (e.g., any of the exemplary soluble tissue factor domains described herein or known in the art) and the first domain of the pair of affinity domains (e.g., any of the exemplary first domains of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the N-terminus directly abuts the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the first chimeric polypeptide further comprises a linker sequence (e.g., any of the linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the C-terminus directly abuts the first target-binding domain (e.g., any of the exemplary target-binding domains described herein or known in the art) or the first domain of the pair of affinity domains (e.g., any of the exemplary first domains of any of the exemplary pairs of affinity domains described herein) in the first chimeric polypeptide. In some embodiments, the first chimeric polypeptide further includes a linker sequence (e.g., any of the exemplary linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one of the one or more additional target-binding domains (e.g., any of the exemplary target-binding domains described herein or known in the art) positioned between the soluble tissue factor domain (e.g., any of the exemplary soluble tissue factor domains described herein) and the first domain of the pair of affinity domains (e.g., any of the first domains described herein or any of the exemplary pairs of affinity domains described herein), directly abuts the soluble tissue factor domain and/or the first domain of the pair of affinity domains. In some embodiments, the first chimeric polypeptide further comprises a linker sequence (e.g., any of the exemplary linker sequences described herein or known in the art) disposed (i) between the soluble tissue factor domain (e.g., any of the exemplary soluble tissue factor domains described herein) and the at least one of the one or more additional target-binding domains (e.g., any of the exemplary target-binding domains described herein or known in the art) positioned between the soluble tissue factor domain (e.g., any of the exemplary soluble tissue factor domains described herein) and the first domain of the pair of affinity domains (e.g., any of the exemplary first domains of any of the exemplary pairs of affinity domains described herein), and/or (ii) between the first domain of the pair of affinity domains and the at least one of the one or more additional target-binding domains positioned between the soluble tissue factor domain and the first domain of the pair of affinity domains.

In some embodiments of any of the multi-chain chimeric polypeptides described herein, the second chimeric polypeptide further includes one or more (e.g., two, three, four, five, six, seven, eight, nine, or ten) additional target-binding domains (e.g., any of the exemplary target-binding domains described herein or known in the art) at the N-terminal end and/or the C-terminal end of the second chimeric polypeptide. In some embodiments, at least one of the one or more additional target-binding domains (e.g., any of the exemplary target-binding domains described herein or known in the art) directly abuts the second domain of the pair of affinity domains (e.g., any of the exemplary second domains of any of the exemplary pairs of affinity domains described herein) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target-binding domains described herein or known in the art) and the second domain of the pair of affinity domains (e.g., any of the second domains described herein of any of the exemplary pairs of affinity domains described herein) in the second chimeric polypeptide. In some embodiments, at least one of the one or more additional target-binding domains (e.g., any of the exemplary target-binding domains described herein or known in the art) directly abuts the second target-binding domain (e.g., any of the target-binding domains described herein or known in the art) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target binding domains described herein or known in the art) and the second target-binding domain (e.g., any of the exemplary target binding domains described herein or known in the art) in the second chimeric polypeptide.

In some embodiments of any of the multi-chain chimeric polypeptides described herein, two or more (e.g., three or more, four or more, five or more, six or more, seven or more, eight or more, nine or more, or ten or more) of the first target-binding domain, the second target-binding domain, and the one or more additional target-binding domains bind specifically to the same antigen. In some embodiments, two or more (e.g., three or more, four or more, five or more, six or more, seven or more, eight or more, nine or more, or ten or more) of the first target-binding domain, the second target-binding domain, and the one or more additional target-binding domains bind specifically to the same epitope. In some embodiments, two or more (e.g., three or more, four or more, five or more, six or more, seven or more, eight or more, nine or more, or ten or more) of the first target-binding domain, the second target-binding domain, and the one or more additional target-binding domains include the same amino acid sequence. In some embodiments, the first target-binding domain, the second target-binding domain, and the one or more additional target-binding domains each bind specifically to the same antigen. In some embodiments, the first target-binding domain, the second target-binding domain, and the one or more additional target-binding domains each bind specifically to the same epitope. In some embodiments, the first target-binding domain, the second target-binding domain, and the one or more additional target-binding domains each include the same amino acid sequence.

In some embodiments of any of the multi-chain chimeric polypeptides described herein, the first target-binding domain, the second target-binding domain, and the one or more additional target-binding domains bind specifically to different antigens. In some embodiments of any of the multi-chain chimeric polypeptides described herein, one or more (e.g., two or more, three or more, four or more, five or more, six or more, seven or more, eight or more, nine or more, or ten or more) of the first target-binding domain, the second target-binding domain, and the one or more target-binding domains is an antigen-binding domain. In some embodiments, the first target-binding domain, the second target-binding domain, and the one or more additional target-binding domains are each an antigen-binding domain (e.g., a scFv or a single-domain antibody).

### Pairs of Affinity Domains

In some embodiments, a multi-chain chimeric polypeptide includes: 1) a first chimeric polypeptide that includes a first domain of a pair of affinity domains, and 2) a second chimeric polypeptide that includes a second domain of a pair of affinity domains such that the first chimeric polypeptide and the second chimeric polypeptide associate through the binding of the first domain and the second domain of the pair of affinity domains. In some embodiments, the pair of affinity domains is a sushi domain from an alpha chain of human IL-15 receptor (IL15Rα) and a soluble IL-15. A sushi domain, also known as a short consensus repeat or type 1 glycoprotein motif, is a common motif in protein-protein interaction. Sushi domains have been identified on a number of protein-binding molecules, including complement components C1r, C1s, factor H, and C2m, as well as the nonimmunologic molecules factor XIII and β2-glycoprotein. A typical Sushi domain has approximately 60 amino acid residues and contains four cysteines (Ranganathan, Pac. Symp Biocomput. 2000:155-67). The first cysteine can form a disulfide bond with the third cysteine, and the second cysteine can form a disulfide bridge with the fourth cysteine. In some embodiments in which one member of the pair of affinity domains is a soluble IL-15, the soluble IL15 has a D8N or D8A amino acid substitution. In some embodiments in which one member of the pair of affinity domains is an alpha chain of human IL-15 receptor (IL15Rα), the human IL15Rα is a mature full-length IL15Ra. In some embodiments, the pair of affinity domains is barnase and barnstar. In some embodiments, the pair of affinity domains is a PKA and an AKAP. In some embodiments, the pair of affinity domains is an adapter/docking tag module based on mutated RNase I fragments (Rossi, Proc Natl Acad Sci USA. 103:6841-6846, 2006; Sharkey et al., Cancer Res. 68:5282-5290, 2008; Rossi et al., Trends Pharmacol Sci. 33:474-481, 2012) or SNARE modules based on interactions of the proteins syntaxin, synaptotagmin, synaptobrevin, and SNAP25 (Deyev et al., Nat Biotechnol. 1486-1492, 2003).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains and the second domain of the pair of affinity domains bind to each other with a dissociation equilibrium constant (K_{D}) of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the first domain of the pair of affinity domains and the second domain of the pair of affinity domains bind to each other with a K_{D} of about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, about 1×10⁻⁹M to about 1×10⁻¹¹ M, about 1×10 M to about 1×10⁻¹² M, about 1×10⁻¹¹M to about 1×10⁻¹³ M, about 1×10⁻⁴ M to about 1×10^{×5} M, about 1×10^{×5} M to about 1×10⁻⁶ M, about 1×10⁻⁶ M to about 1×10⁻⁷ M, about 1×10⁻⁷ M to about 1×10⁻⁸ M, about 1×10⁻⁸M to about 1×10⁻⁹ M, about 1×10⁻⁹M to about 1×10⁻¹⁰ M, about 1×10⁻¹⁰ M to about 1×10⁻¹¹ about 1×10⁻¹¹M to about 1×10¹² M, or about 1×10¹² M to about 1×10⁻¹³ M (inclusive). Any of a variety of different methods known in the art can be used to determine the K_{D} value of the binding of the first domain of the pair of affinity domains and the second domain of the pair of affinity domains (e.g., an electrophoretic mobility shift assay, a filter binding assay, surface plasmon resonance, and a biomolecular binding kinetics assay, etc.).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains, the second domain of the pair of affinity domains, or both is about 10 to 100 amino acids in length. For example, a first domain of a pair of affinity domains, a second domain of a pair of affinity domains, or both can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a first domain of a pair of affinity domains, a second domain of a pair of affinity domains, or both is about 10, 15, 20, 25, 30, 35, 40, 45, 50, 55, 60, 65, 70, 75, 80, 85, 90, 95, or 100 amino acids in length.

In some embodiments, any of the first and/or second domains of a pair of affinity domains disclosed herein can include one or more additional amino acids (e.g., 1, 2, 3, 5, 6, 7, 8, 9, 10, or more amino acids) at its N-terminus and/or C-terminus, so long as the function of the first and/or second domains of a pair of affinity domains remains intact. For example, a sushi domain from an alpha chain of human IL-15 receptor (IL15Rα) can include one or more additional amino acids at the N-terminus and/or the C-terminus, while still retaining the ability to bind to a soluble IL-15. Additionally or alternatively, a soluble IL-15 can include one or more additional amino acids at the N-terminus and/or the C-terminus, while still retaining the ability to bind to a sushi domain from an alpha chain of human IL-15 receptor (IL15Rα).

A non-limiting example of a sushi domain from an alpha chain of IL-15 receptor alpha (IL15Rα) can include a sequence that is at least 70% identical, at least 75% identical, at least 80% identical, at least 85% identical, at least 90% identical, at least 95% identical, at least 99% identical, or 100% identical to ITCPPPMSVEHADIWVKSYSLYSRERYICNSGFKRKAGTSSLTECVLNKATNVAH WTTPSLKCIR (SEQ ID NO: 16). In some embodiments, a sushi domain from an alpha chain of IL15Rα can be encoded by a nucleic acid including

(SEQ ID NO: 17)
ATTACATGCCCCCCTCCCATGAGCGTGGAGCACGCCGACATCTGGGTGA
AGAGCTATAGCCTCTACAGCCGGGAGAGGTATATCTGTAACAGCGGCTT
CAAGAGGAAGGCCGGCACCAGCAGCCTCACCGAGTGCGTGCTGAATAAG
GCTACCAACGTGGCTCACTGGACAACACCCTCTTTAAAGTGCATCCGG.

In some embodiments, a soluble IL-15 can include a sequence that is at least 70% identical, at least 75% identical, at least 80% identical, at least 85% identical, at least 90% identical, at least 95% identical, at least 99% identical, or 100% identical to NWVNVISDLKKIEDLIQSMHIDATLYTESDVHP SCKVTAMKCFLLELQVISLESGD ASIHDTVENLIILANNSLSSNGNVTESGCKECEELEEKNIKEFLQSFVHIVQ1VIFINT S (SEQ ID NO: 18). In some embodiments, a soluble IL-15 can be encoded by a nucleic acid including the sequence of

(SEQ ID NO: 19)
AACTGGGTGAACGTCATCAGCGATTTAAAGAAGATCGAAGATTTAATTC
AGTCCATGCATATCGACGCCACTTTATACACAGAATCCGACGTGCACCC
CTCTTGTAAGGTGACCGCCATGAAATGTTTTTTACTGGAGCTGCAAGTT
ATCTCTTTAGAGAGCGGAGACGCTAGCATCCACGACACCGTGGAGAATT
TAATCATTTTAGCCAATAACTCTTTATCCAGCAACGGCAACGTGACAGA
GTCCGGCTGCAAGGAGTGCGAAGAGCTGGAGGAGAAGAACATCAAGGAG
TTTCTGCAATCCTTTGTGCACATTGTCCAGATGTTCATCAATACCTCC.

In some embodiments, a soluble IL-15 can include a D8N amino acid substitution. In some embodiments, the soluble IL-15 with D8N mutant (IL15D8N) can include a sequence that is at least 70% identical, at least 75% identical, at least 80% identical, at least 85% identical, at least 90% identical, at least 92% identical, at least 94% identical, at least 95% identical, at least 96% identical, at least 98% identical, at least 99% identical, or 100% identical to NWVNVISNLKKIEDLIQ SMIIIDATLYTE SD VHP SCKVTAMKCFLLELQVISLESGD A SIHD TVENLIILANNSLSSNGNVTESGCKECEELEEKNIKEFLQSFVHIVQMFINT S (SEQ ID NO: 70). In some embodiments, the soluble IL-15 with D8N mutant (IL15D8N) can be encoded by a nucleic acid including the sequence of

(SEQ ID NO: 71)
AACTGGGTGAATGTAATAAGTAATTTGAAAAAAATTGAAGATCTTATTC
AATCTATGCATATTGATGCTACTTTATATACGGAAAGTGATGTTCACCCC
AGTTGCAAAGTAACAGCAATGAAGTGCTTTCTCTTGGAGTTACAAGTTAT
TTCACTTGAGTCCGGAGATGCAAGTATTCATGATACAGTAGAAAATCTGA
TCATCCTAGCAAACAACAGTTTGTCTTCTAATGGGAATGTAACAGAATCT
GGATGCAAAGAATGTGAGGAACTGGAGGAAAAAAATATTAAAGAATTTTT
GCAGAGTTTTGTACATATTGTCCAAATGTTCATCAACACTTCT.

### Signal Sequence

In some embodiments, a multi-chain chimeric polypeptide includes a first chimeric polypeptide that includes a signal sequence at its N-terminal end. In some embodiments, a multi-chain chimeric polypeptide includes a second chimeric polypeptide that includes a signal sequence at its N-terminal end. In some embodiments, both the first chimeric polypeptide of a multi-chain chimeric polypeptide and a second chimeric polypeptide of the multi-chain chimeric polypeptide include a signal sequence. As will be understood by those of ordinary skill in the art, a signal sequence is an amino acid sequence that is present at the N-terminus of a number of endogenously produced proteins that directs the protein to the secretory pathway (e.g., the protein is directed to reside in certain intracellular organelles, to reside in the cell membrane, or to be secreted from the cell). Signal sequences are heterogeneous and differ greatly in their primary amino acid sequences. However, signal sequences are typically 16 to 30 amino acids in length and include a hydrophilic, usually positively charged N-terminal region, a central hydrophobic domain, and a C-terminal region that contains the cleavage site for signal peptidase.

In some embodiments, a first chimeric polypeptide of a multi-chain chimeric polypeptide, a second chimeric polypeptide of the multi-chain chimeric polypeptide, or both includes a signal sequence having an amino acid sequence MKWVTFISLLFLFSSAYS (SEQ ID NO: 20). In some embodiments, a first chimeric polypeptide of a multi-chain chimeric polypeptide, a second chimeric polypeptide of the multi-chain chimeric polypeptide, or both includes a signal sequence encoded by the nucleic acid sequence

(SEQ ID NO: 21)
ATGAAATGGGTGACCTTTATTTCTTTACTGTTCCTCTTTAGCAGCGCCTA
CTCC,
(SEQ ID NO: 22)
ATGAAGTGGGTCACATTTATCTCTTTACTGTTCCTCTTCTCCAGCGCCTA
CAGC,
or
(SEQ ID NO: 23)
ATGAAATGGGTGACCTTTATTTCTTTACTGTTCCTCTTTAGCAGCGCCTA
CTCC.

In some embodiments, a first chimeric polypeptide of a multi-chain chimeric polypeptide, a second chimeric polypeptide of the multi-chain chimeric polypeptide, or both includes a signal sequence having an amino acid sequence MKCLLYLAFLFLGVNC (SEQ ID NO: 24). In some embodiments, a first chimeric polypeptide of a multi-chain chimeric polypeptide, a second chimeric polypeptide of the multi-chain chimeric polypeptide, or both includes a signal sequence having an amino acid sequence MGQIVT1VIFEALPHIIDEVINIVIIVLIIITSIKAVYNFATCGILALVSFLFLAGRSCG (SEQ ID NO: 25). In some embodiments, a first chimeric polypeptide of a multi-chain chimeric polypeptide, a second chimeric polypeptide of the multi-chain chimeric polypeptide, or both includes a signal sequence having an amino acid sequence

(SEQ ID NO: 26)
MPNHQSGSPTGSSDLLLSGKKQRPHLALRRKRRREMRKINRKVRRMNLAP
IKEKTAWQHLQALISEAEEVLKTSQTPQNSLTLFLALLSVLGPPVTG.

In some embodiments, a first chimeric polypeptide of a multi-chain chimeric polypeptide, a second chimeric polypeptide of the multi-chain chimeric polypeptide, or both includes a signal sequence having an amino acid sequence MDSKGSSQKGSRLLLLLVVSNLLLCQGVVS (SEQ ID NO: 27). Those of ordinary skill in the art will be aware of other appropriate signal sequences for use in a first chimeric polypeptide and/or a second chimeric polypeptide of multi-chain chimeric polypeptides described herein.

In some embodiments, a first chimeric polypeptide of a multi-chain chimeric polypeptide, a second chimeric polypeptide of the multi-chain chimeric polypeptide, or both includes a signal sequence that is about 10 to 100 amino acids in length. For example, a signal sequence can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a signal sequence is about 10, 15, 20, 25, 30, 35, 40, 45, 50, 55, 60, 65, 70, 75, 80, 85, 90, 95, or 100 amino acids in length.

In some embodiments, any of the signal sequences disclosed herein can include one or more additional amino acids (e.g., 1, 2, 3, 5, 6, 7, 8, 9, 10, or more amino acids) at its N-terminus and/or C-terminus, so long as the function of the signal sequence remains intact. For example, a signal sequence having the amino acid sequence MKCLLYLAFLFLGVNC (SEQ ID NO: 28) can include one or more additional amino acids at the N-terminus or C-terminus, while still retaining the ability to direct a first chimeric polypeptide of a multi-chain chimeric polypeptide, a second chimeric polypeptide of the multi-chain chimeric polypeptide, or both to the secretory pathway.

In some embodiments, a first chimeric polypeptide of a multi-chain chimeric polypeptide, a second chimeric polypeptide of the multi-chain chimeric polypeptide, or both includes a signal sequence that directs the multi-chain chimeric polypeptide into the extracellular space. Such embodiments are useful in producing multi-chain chimeric polypeptides that are relatively easy to be isolated and/or purified.

### Peptide Tags

In some embodiments, a multi-chain chimeric polypeptide includes a first chimeric polypeptide that includes a peptide tag (e.g., at the N-terminal end or the C-terminal end of the first chimeric polypeptide). In some embodiments, a multi-chain chimeric polypeptide includes a second chimeric polypeptide that includes a peptide tag (e.g., at the N-terminal end or the C-terminal end of the second chimeric polypeptide). In some embodiments, both the first chimeric polypeptide of a multi-chain chimeric polypeptide and a second chimeric polypeptide of the multi-chain chimeric polypeptide include a peptide tag. In some embodiments, a first chimeric polypeptide of a multi-chain chimeric polypeptide, a second chimeric polypeptide of the multi-chain chimeric polypeptide, or both include two or more peptide tags.

Exemplary peptide tags that can be included in a first chimeric polypeptide of a multi-chain chimeric polypeptide, a second chimeric polypeptide of the multi-chain chimeric polypeptide, or both include, without limitation, AviTag (GLNDIFEAQKIEWHE; SEQ ID NO: 29), a calmodulin-tag (KRRWKKNFIAVSAANRFKKISSSGAL; SEQ ID NO: 30), a polyglutamate tag (EEEEEE; SEQ ID NO: 31), an E-tag (GAPVPYPDPLEPR; SEQ ID NO: 32), a FLAG-tag (DYKDDDDK; SEQ ID NO: 33), an HA-tag, a peptide from hemagglutinin (YPYDVPDYA; SEQ ID NO: 34), a his-tag (HHHHH (SEQ ID NO: 35); HI IH (SEQ ID NO: 36); HHHHHHH (SEQ ID NO: 37); HHHHHHHH (SEQ ID NO: 38); HHHHHHHHH (SEQ ID NO: 39); or HHHHHHHHHH (SEQ ID NO: 40)), a myc-tag (EQKLISEEDL; SEQ ID NO: 41), NE-tag (TKENPRSNQEESYDDNES; SEQ ID NO: 42), S-tag, (KETAAAKFERQHMDS; SEQ ID NO: 43), SBP-tag (MDEKTTGWRGGHVVEGLAGELEQLRARLEHHPQGQREP; SEQ ID NO: 44), Softag 1 (SLAELLNAGLGGS; SEQ ID NO: 45), Softag 3 (TQDPSRVG; SEQ ID NO: 46), Spot-tag (PDRVRAVSHWSS; SEQ ID NO: 47), Strep-tag (WSHPQFEK; SEQ ID NO: 48), TC tag (CCPGCC; SEQ ID NO: 49), Ty tag (EVHTNQDPLD; SEQ ID NO: 50), V5 tag (GKPIPNPLLGLDST; SEQ ID NO: 51), VSV-tag (YTDIEMNRLGK; SEQ ID NO: 52), and Xpress tag (DLYDDDDK; SEQ ID NO: 53). In some embodiments, tissue factor protein is a peptide tag.

Peptide tags that can be included in a first chimeric polypeptide of a multi-chain chimeric polypeptide, a second chimeric polypeptide of the multi-chain chimeric polypeptide, or both can be used in any of a variety of applications related to the multi-chain chimeric polypeptide. For example, a peptide tag can be used in the purification of a multi-chain chimeric polypeptide. As one non-limiting example, a first chimeric polypeptide of a multi-chain chimeric polypeptide (e.g., a recombinantly expressed first chimeric polypeptide), a second chimeric polypeptide of the multi-chain chimeric polypeptide (e.g., a recombinantly expressed second chimeric polypeptide), or both can include a myc tag; the multi-chain chimeric polypeptide that includes the myc-tagged first chimeric polypeptide, the myc-tagged second chimeric polypeptide, or both can be purified using an antibody that recognizes the myc tag(s). One non-limiting example of an antibody that recognizes a myc tag is 9E10, available from the non-commercial Developmental Studies Hybridoma Bank. As another non-limiting example, a first chimeric polypeptide of a multi-chain chimeric polypeptide (e.g., a recombinantly expressed first chimeric polypeptide), a second chimeric polypeptide of the multi-chain chimeric polypeptide (e.g., a recombinantly expressed second chimeric polypeptide), or both can include a histidine tag; the multi-chain chimeric polypeptide that includes the histidine-tagged first chimeric polypeptide, the histidine-tagged second chimeric polypeptide, or both can be purified using a nickel or cobalt chelate. Those of ordinary skill in the art will be aware of other suitable tags and agent that bind those tags for use in purifying multi-chain chimeric polypeptide. In some embodiments, a peptide tag is removed from the first chimeric polypeptide and/or the second chimeric polypeptide of the multi-chain chimeric polypeptide after purification. In some embodiments, a peptide tag is not removed from the first chimeric polypeptide and/or the second chimeric polypeptide of the multi-chain chimeric polypeptide after purification.

Peptide tags that can be included in a first chimeric polypeptide of a multi-chain chimeric polypeptide, a second chimeric polypeptide of the multi-chain chimeric polypeptide, or both can be used, for example, in immunoprecipitation of the multi-chain chimeric polypeptide, imaging of the multi-chain chimeric polypeptide (e.g., via Western blotting, ELISA, flow cytometry, and/or immunocytochemistry), and/or solubilization of the multi-chain chimeric polypeptide.

In some embodiments, a first chimeric polypeptide of a multi-chain chimeric polypeptide, a second chimeric polypeptide of the multi-chain chimeric polypeptide, or both includes a peptide tag that is about 10 to 100 amino acids in length. For example, a peptide tag can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a peptide tag is about 10, 15, 20, 25, 30, 35, 40, 45, 50, 55, 60, 65, 70, 75, 80, 85, 90, 95, or 100 amino acids in length.

Peptide tags included in a first chimeric polypeptide of a multi-chain chimeric polypeptide, a second chimeric polypeptide of the multi-chain chimeric polypeptide, or both can be of any suitable length. For example, peptide tags can be 5, 6, 7, 8, 9, 10, 11, 12, 13, 14, 15, 16, 17, 18, 19, 20, or more amino acids in length. In embodiments in which a multi-chain chimeric polypeptide includes two or more peptide tags, the two or more peptide tags can be of the same or different lengths. In some embodiments, any of the peptide tags disclosed herein may include one or more additional amino acids (e.g., 1, 2, 3, 5, 6, 7, 8, 9, 10, or more amino acids) at the N-terminus and/or C-terminus, so long as the function of the peptide tag remains intact. For example, a myc tag having the amino acid sequence EQKLISEEDL (SEQ ID NO: 54) can include one or more additional amino acids (e.g., at the N-terminus and/or the C- terminus of the peptide tag), while still retaining the ability to be bound by an antibody.

### Exemplary Multi-Chain Chimeric Polypeptides

In some embodiments of any of the multi-chain chimeric polypeptides described herein, the first target-binding domain and the second targeting-binding domain each independently bind specifically to TGF-β. In some examples of these multi-chain chimeric polypeptides, the first target-binding domain and the soluble tissue factor domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble tissue factor domain in the first chimeric polypeptide.

In some embodiments of these multi-chain chimeric polypeptides, the soluble tissue factor domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble tissue factor domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble tissue factor domain can be any of the exemplary soluble tissue factor domains described herein. In some embodiments of these multi-chain chimeric polypeptides, the pair of affinity domains can be any of the exemplary pairs of affinity domains described herein.

In some embodiments of these multi-chain chimeric polypeptides, the first target-binding domain and the second target-binding domain each independently bind specifically to TGF-β. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of these multi-chain chimeric polypeptides, the first target-binding domain and the second target-binding domain is a soluble TGF-β receptor (e.g., a soluble TGFβRII receptor, e.g., a soluble human TGFβRII). In some embodiments of these multi-chain chimeric polypeptides, the soluble human TGFRβRII includes a first sequence of soluble human TGFRβRII and a second sequence of soluble human TGFRβRII. In some embodiments of these multi-chain chimeric polypeptides, the soluble human TGFRβRII includes a linker disposed between the first sequence of soluble human TGFRβRII and the second sequence of soluble human TGFRβRII. In some examples of these multi-chain chimeric polypeptides, the linker includes the sequence GGGGSGGGGSGGGGS (SEQ ID NO: 3).

In some embodiments of these multi-chain chimeric polypeptides, the first sequence of soluble human TGFRβRII receptor comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 2)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPD.

In some embodiments of these multi-chain chimeric polypeptides, the second sequence of soluble human TGFRβRII receptor comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 2)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPD.

In some embodiments of these multi-chain chimeric polypeptides, the first sequence of soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 55)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGAT.

In some embodiments of these multi-chain chimeric polypeptides, the second sequence of soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 56)
ATTCCTCCCCACGTGCAGAAGAGCGTGAATAATGACATGATCGTGACCG
ATAACAATGGCGCCGTGAAATTTCCCCAGCTGTGCAAATTCTGCGATGT
GAGGTTTTCCACCTGCGACAACCAGAAGTCCTGTATGAGCAACTGCTCC
ATCACCTCCATCTGTGAGAAGCCTCAGGAGGTGTGCGTGGCTGTCTGGC
GGAAGAATGACGAGAATATCACCCTGGAAACCGTCTGCCACGATCCCAA
GCTGCCCTACCACGATTTCATCCTGGAAGACGCCGCCAGCCCTAAGTGC
ATCATGAAAGAGAAAAAGAAGCCTGGCGAGACCTTTTTCATGTGCTCCT
GCAGCAGCGACGAATGCAACGACAATATCATCTTTAGCGAGGAATACAA
TACCAGCAACCCCGAC.

In some embodiments of these multi-chain chimeric polypeptides, the soluble TGF-β receptor includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 4)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPD.

In some embodiments of these multi-chain chimeric polypeptides, the soluble

TGF-β receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 57)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCTCCATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGAC.

In some embodiments, the first chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 15 100% identical) to:

(SEQ ID NO: 6)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPDSGTTNTV
AAYNLTWKSTNFKTILEWEPKPVNQVYTVQISTKSGDWKSKCFYTTDTE
CDLTDEIVKDVKQTYLARVFSYPAGNVESTGSAGEPLYENSPEFTPYLE
TNLGQPTIQSFEQVGTKVNVTVEDERTLVRRNNTFLSLRDVFGKDLIYT
LYYWKSSSSGKKTAKTNTNEFLIDVDKGENYCFSVQAVIPSRTVNRKST
DSPVECMGQEKGEFRENWVNVISDLKKIEDLIQSMHIDATLYTESDVHP
SCKVTAMKCFLLELQVISLESGDASIHDTVENLIILANNSLSSNGNVTE
SGCKECEELEEKNIKEFLQSFVHIVQMFINTS.

In some embodiments, a first chimeric polypeptide is encoded by a sequence that is at least 80% identical (e.g., at least 82% identical, at least 84% identical, at least 86% 30 identical, at least 88% identical, at least 90% identical, at least 92% identical, at least 94% identical, at least 96% identical, at least 98% identical, at least 99% identical, or 100% identical) to:

(SEQ ID NO: 58)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCTCCATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGACAGCGGCACAACCAACACAGTC
GCTGCCTATAACCTCACTTGGAAGAGCACCAACTTCAAAACCATCCTCG
AATGGGAACCCAAACCCGTTAACCAAGTTTACACCGTGCAGATCAGCAC
CAAGTCCGGCGACTGGAAGTCCAAATGTTTCTATACCACCGACACCGAG
TGCGATCTCACCGATGAGATCGTGAAAGATGTGAAACAGACCTACCTCG
CCCGGGTGTTTAGCTACCCCGCCGGCAATGTGGAGAGCACTGGTTCCGC
TGGCGAGCCTTTATACGAGAACAGCCCCGAATTTACCCCTTACCTCGAG
ACCAATTTAGGACAGCCCACCATCCAAAGCTTTGAGCAAGTTGGCACAA
AGGTGAATGTGACAGTGGAGGACGAGCGGACTTTAGTGCGGCGGAACAA
CACCTTTCTCAGCCTCCGGGATGTGTTCGGCAAAGATTTAATCTACACA
CTGTATTACTGGAAGTCCTCTTCCTCCGGCAAGAAGACAGCTAAAACCA
ACACAAACGAGTTTTTAATCGACGTGGATAAAGGCGAAAACTACTGTTT
CAGCGTGCAAGCTGTGATCCCCTCCCGGACCGTGAATAGGAAAAGCACC
GATAGCCCCGTTGAGTGCATGGGCCAAGAAAAGGGCGAGTTCCGGGAGA
AACTGGGTGAACGTCATCAGCGATTTAAAGAAGATCGAAGATTTAATTC
AGTCCATGCATATCGACGCCACTTTATACACAGAATCCGACGTGCCCCC
TCTTGTAAGGTGACCGCCATGAAATGTTTTTTACTGGAGCTGCAAGTTA
TCTCTTTAGAGAGCGGAGACGCTAGCATCCACGACACCGTGGAGAATTT
AATCATTTTAGCCAATAACTCTTTATCCAGCAACGGCAACGTGACAGAG
TCCGGCTGCAAGGAGTGCGAAGAGCTGGAGGAGAAGAACATCAAGGAGT
TTCTGCAATCCTTTGTGCACATTGTCCAGATGTTCATCAATACCTCC.

In some embodiments, a first chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 7)
MKWVTFISLLFLFSSAYSIPPHVQKSVNNDMIVTDNNGAVKFPQLCKFCD
VRFSTCDNQKSCMSNCSITSICEKPQEVCVAVWRKNDENITLETVCHDPK
LPYHDFILEDAASPKCIMKEKKKPGETFFMCSCSSDECNDNIIFSEEYNT
SNPDGGGGSGGGGSGGGGSIPPHVQKSVNNDMIVTDNNGAVKFPQLCKFC
DVRFSTCDNQKSCMSNCSITSICEKPQEVCVAVWRKNDENITLETVCHDP
KLPYHDFILEDAASPKCIMKEKKKPGETFFMCSCSSDECNDNIIFSEEYN
TSNPDSGTTNTVAAYNLTWKSTNFKTILEWEPKPVNQVYTVQISTKSGDW
KSKCFYTTDTECDLTDEIVKDVKQTYLARVFSYPAGNVESTGSAGEPLYE
NSPEFTPYLETNLGQPTIQSFEQVGTKVNVTVEDERTLVRRNNTFLSLRD
VFGKDLIYTLYYWKSSSSGKKTAKTNTNEFLIDVDKGENYCFSVQAVIPS
RTVNRKSTDSPVECMGQEKGEFRENWVNVISDLKKIEDLIQSMHIDATLY
TESDVHPSCKVTAMKCFLLELQVISLESGDASIHDTVENLIILANNSLSS
NGNVTESGCKECEELEEKNIKEFLQSFVHIVQMFINTS.

In some embodiments, a first chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 59)
ATGAAGTGGGTGACCTTCATCAGCCTGCTGTTCCTGTTCTCCAGCGCCT
ACTCCATCCCCCCCCATGTGCAAAAGAGCGTGAACAACGATATGATCGT
GACCGACAACAACGGCGCCGTGAAGTTTCCCCAGCTCTGCAAGTTCTGC
GATGTCAGGTTCAGCACCTGCGATAATCAGAAGTCCTGCATGTCCAACT
GCAGCATCACCTCCATCTGCGAGAAGCCCCAAGAAGTGTGCGTGGCCGT
GTGGCGGAAAAATGACGAGAACATCACCCTGGAGACCGTGTGTCACGAC
CCCAAGCTCCCTTATCACGACTTCATTCTGGAGGACGCTGCCTCCCCCA
AATGCATCATGAAGGAGAAGAAGAAGCCCGGAGAGACCTTCTTTATGTG
TTCCTGTAGCAGCGACGAGTGTAACGACAACATCATCTTCAGCGAAGAG
TACAACACCAGCAACCCTGATGGAGGTGGCGGATCCGGAGGTGGAGGTT
CTGGTGGAGGTGGGAGTATTCCTCCCCACGTGCAGAAGAGCGTGAATAA
TGACATGATCGTGACCGATAACAATGGCGCCGTGAAATTTCCCCAGCTG
TGCAAATTCTGCGATGTGAGGTTTTCCACCTGCGACAACCAGAAGTCCT
GTATGAGCAACTGCTCCATCACCTCCATCTGTGAGAAGCCTCAGGAGGT
GTGCGTGGCTGTCTGGCGGAAGAATGACGAGAATATCACCCTGGAAACC
GTCTGCCACGATCCCAAGCTGCCCTACCACGATTTCATCCTGGAAGACG
CCGCCAGCCCTAAGTGCATCATGAAAGAGAAAAAGAAGCCTGGCGAGAC
CTTTTTCATGTGCTCCTGCAGCAGCGACGAATGCAACGACAATATCATC
TTTAGCGAGGAATACAATACCAGCAACCCCGACAGCGGCACAACCAACA
CAGTCGCTGCCTATAACCTCACTTGGAAGAGCACCAACTTCAAAACCAT
CCTCGAATGGGAACCCAAACCCGTTAACCAAGTTTACACCGTGCAGATC
AGCACCAAGTCCGGCGACTGGAAGTCCAAATGTTTCTATACCACCGACA
CCGAGTGCGATCTCACCGATGAGATCGTGAAAGATGTGAAACAGACCTA
CCTCGCCCGGGTGTTTAGCTACCCCGCCGGCAATGTGGAGAGCACTGGT
TCCGCTGGCGAGCCTTTATACGAGAACAGCCCCGAATTTACCCCTTACC
TCGAGACCAATTTAGGACAGCCCACCATCCAAAGCTTTGAGCAAGTTGG
CACAAAGGTGAATGTGACAGTGGAGGACGAGCGGACTTTAGTGCGGCGG
AACAACACCTTTCTCAGCCTCCGGGATGTGTTCGGCAAAGATTTAATCT
ACACACTGTATTACTGGAAGTCCTCTTCCTCCGGCAAGAAGACAGCTAA
AACCAACACAAACGAGTTTTTAATCGACGTGGATAAAGGCGAAAACTAC
TGTTTCAGCGTGCAAGCTGTGATCCCCTCCCGGACCGTGAATAGGAAAA
GCACCGATAGCCCCGTTGAGTGCATGGGCCAAGAAAAGGGCGAGTTCCG
GGAGAACTGGGTGAACGTCATCAGCGATTTAAAGAAGATCGAAGATTTA
ATTCAGTCCATGCATATCGACGCCACTTTATACACAGAATCCGACGTGC
ACCCCTCTTGTAAGGTGACCGCCATGAAATGTTTTTTACTGGAGCTGCA
AGTTATCTCTTTAGAGAGCGGAGACGCTAGCATCCACGACACCGTGGAG
AATTTAATCATTTTAGCCAATAACTCTTTATCCAGCAACGGCAACGTGA
CAGAGTCCGGCTGCAAGGAGTGCGAAGAGCTGGAGGAGAAGAACATCAA
GGAGTTTCTGCAATCCTTTGTGCACATTGTCCAGATGTTCATCAATACC
TCC.

In some embodiments, the second chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 5)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPDITCPPPM
SVEHADIWVKSYSLYSRERYICNSGFKRKAGTSSLTECVLNKATNVAHW
TTPSLKCIR.

In some embodiments, a second chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 60)
ATCCCCCCCCATGTGCAAAAGAGCGTGAACAACGATATGATCGTGACCG
ACAACAACGGCGCCGTGAAGTTTCCCCAGCTCTGCAAGTTCTGCGATGT
CAGGTTCAGCACCTGCGATAATCAGAAGTCCTGCATGTCCAACTGCAGC
ATCACCTCCATCTGCGAGAAGCCCCAAGAAGTGTGCGTGGCCGTGTGGC
GGAAAAATGACGAGAACATCACCCTGGAGACCGTGTGTCACGACCCCAA
GCTCCCTTATCACGACTTCATTCTGGAGGACGCTGCCTCCCCCAAATGC
ATCATGAAGGAGAAGAAGAAGCCCGGAGAGACCTTCTTTATGTGTTCCT
GTAGCAGCGACGAGTGTAACGACAACATCATCTTCAGCGAAGAGTACAA
CACCAGCAACCCTGATGGAGGTGGCGGATCCGGAGGTGGAGGTTCTGGT
GGAGGTGGGAGTATTCCTCCCCACGTGCAGAAGAGCGTGAATAATGACA
TGATCGTGACCGATAACAATGGCGCCGTGAAATTTCCCCAGCTGTGCAA
ATTCTGCGATGTGAGGTTTTCCACCTGCGACAACCAGAAGTCCTGTATG
AGCAACTGCTCCATCACCTCCATCTGTGAGAAGCCTCAGGAGGTGTGCG
TGGCTGTCTGGCGGAAGAATGACGAGAATATCACCCTGGAAACCGTCTG
CCACGATCCCAAGCTGCCCTACCACGATTTCATCCTGGAAGACGCCGCC
AGCCCTAAGTGCATCATGAAAGAGAAAAAGAAGCCTGGCGAGACCTTTT
TCATGTGCTCCTGCAGCAGCGACGAATGCAACGACAATATCATCTTTAG
CGAGGAATACAATACCAGCAACCCCGACATTACATGCCCCCCTCCCATG
AGCGTGGAGCACGCCGACATCTGGGTGAAGAGCTATAGCCTCTACAGCC
GGGAGAGGTATATCTGTAACAGCGGCTTCAAGAGGAAGGCCGGCACCAG
CAGCCTCACCGAGTGCGTGCTGAATAAGGCTACCAACGTGGCTCACTGG
ACAACACCCTCTTTAAAGTGCATCCGG.

In some embodiments, a second chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 8)
MKWVTFISLLFLFSSAYSIPPHVQKSVNNDMIVTDNNGAVKFPQLCKFC
DVRFSTCDNQKSCMSNCSITSICEKPQEVCVAVWRKNDENITLETVCHD
PKLPYHDFILEDAASPKCIMKEKKKPGETFFMCSCSSDECNDNIIFSEE
YNTSNPDGGGGSGGGGSGGGGSIPPHVQKSVNNDMIVTDNNGAVKFPQL
CKFCDVRFSTCDNQKSCMSNCSITSICEKPQEVCVAVWRKNDENITLET
VCHDPKLPYHDFILEDAASPKCIMKEKKKPGETFFMCSCSSDECNDNII
FSEEYNTSNPDITCPPPMSVEHADIWVKSYSLYSRERYICNSGFKRKAG
TSSLTECVLNKATNVAHWTTPSLKCIR.

In some embodiments, a second chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 61)
ATGAAGTGGGTGACCTTCATCAGCCTGCTGTTCCTGTTCTCCAGCGCCT
ACTCCATCCCCCCCCATGTGCAAAAGAGCGTGAACAACGATATGATCGT
GACCGACAACAACGGCGCCGTGAAGTTTCCCCAGCTCTGCAAGTTCTGC
GATGTCAGGTTCAGCACCTGCGATAATCAGAAGTCCTGCATGTCCAACT
GCAGCATCACCTCCATCTGCGAGAAGCCCCAAGAAGTGTGCGTGGCCGT
GTGGCGGAAAAATGACGAGAACATCACCCTGGAGACCGTGTGTCACGAC
CCCAAGCTCCCTTATCACGACTTCATTCTGGAGGACGCTGCCTCCCCCA
AATGCATCATGAAGGAGAAGAAGAAGCCCGGAGAGACCTTCTTTATGTG
TTCCTGTAGCAGCGACGAGTGTAACGACAACATCATCTTCAGCGAAGAG
TACAACACCAGCAACCCTGATGGAGGTGGCGGATCCGGAGGTGGAGGTT
CTGGTGGAGGTGGGAGTATTCCTCCCCACGTGCAGAAGAGCGTGAATAA
TGACATGATCGTGACCGATAACAATGGCGCCGTGAAATTTCCCCAGCTG
TGCAAATTCTGCGATGTGAGGTTTTCCACCTGCGACAACCAGAAGTCCT
GTATGAGCAACTGCTCCATCACCTCCATCTGTGAGAAGCCTCAGGAGGT
GTGCGTGGCTGTCTGGCGGAAGAATGACGAGAATATCACCCTGGAAACC
GTCTGCCACGATCCCAAGCTGCCCTACCACGATTTCATCCTGGAAGACG
CCGCCAGCCCTAAGTGCATCATGAAAGAGAAAAAGAAGCCTGGCGAGAC
CTTTTTCATGTGCTCCTGCAGCAGCGACGAATGCAACGACAATATCATC
TTTAGCGAGGAATACAATACCAGCAACCCCGACATTACATGCCCCCCTC
CCATGAGCGTGGAGCACGCCGACATCTGGGTGAAGAGCTATAGCCTCTA
CAGCCGGGAGAGGTATATCTGTAACAGCGGCTTCAAGAGGAAGGCCGGC
ACCAGCAGCCTCACCGAGTGCGTGCTGAATAAGGCTACCAACGTGGCTC
ACTGGACAACACCCTCTTTAAAGTGCATCCGG.

In some embodiments, the first chimeric polypeptide can include a sequence that 10 is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 62)
IPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKC
IMKEKKKPGETFFMCSCSSDECNDNIIFSEEYNTSNPDGGGGSGGGGSG
GGGSIPPHVQKSVNNDMIVTDNNGAVKFPQLCKFCDVRFSTCDNQKSCM
SNCSITSICEKPQEVCVAVWRKNDENITLETVCHDPKLPYHDFILEDAA
SPKCIMKEKKKPGETFFMCSCSSDECNDNIIFSEEYNTSNPDITCPPPM
SVEHADIWVKSYSLYSRERYICNSGFKRKAGTSSLTECVLNKATNVAHW
TTPSLKCIR .

In some embodiments, a first chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at 25 least 98% identical, at least 99% identical, or 100% identical) to:

(SEQ ID NO: 63)
ATCCCCCCCCATGTGCAAAAGAGCGTGAACAACGA
TATGATCGTGACCGACAACAACGGCGCCGTGAAGT
TTCCCCAGCTCTGCAAGTTCTGCGATGTCAGGTTC
AGCACCTGCGATAATCAGAAGTCCTGCATGTCCAA
CTGCAGCATCACCTCCATCTGCGAGAAGCCCCAAG
AAGTGTGCGTGGCCGTGTGGCGGAAAAATGACGAG
AACATCACCCTGGAGACCGTGTGTCACGACCCCAA
GCTCCCTTATCACGACTTCATTCTGGAGGACGCTG
CCTCCCCCAAATGCATCATGAAGGAGAAGAAGAAG
CCCGGAGAGACCTTCTTTATGTGTTCCTGTAGCAG
CGACGAGTGTAACGACAACATCATCTTCAGCGAAG
AGTACAACACCAGCAACCCTGATGGAGGTGGCGGA
TCCGGAGGTGGAGGTTCTGGTGGAGGTGGGAGTAT
TCCTCCCCACGTGCAGAAGAGCGTGAATAATGACA
TGATCGTGACCGATAACAATGGCGCCGTGAAATTT
CCCCAGCTGTGCAAATTCTGCGATGTGAGGTTTTC
CACCTGCGACAACCAGAAGTCCTGTATGAGCAACT
GCTCCATCACCTCCATCTGTGAGAAGCCTCAGGAG
GTGTGCGTGGCTGTCTGGCGGAAGAATGACGAGAA
TATCACCCTGGAAACCGTCTGCCACGATCCCAAGC
TGCCCTACCACGATTTCATCCTGGAAGACGCCGCC
AGCCCTAAGTGCATCATGAAAGAGAAAAAGAAGCC
TGGCGAGACCTTTTTCATGTGCTCCTGCAGCAGCG
ACGAATGCAACGACAATATCATCTTTAGCGAGGAA
TACAATACCAGCAACCCCGACATTACATGCCCCCC
TCCCATGAGCGTGGAGCACGCCGACATCTGGGTGA
AGAGCTATAGCCTCTACAGCCGGGAGAGGTATATC
TGTAACAGCGGCTTCAAGAGGAAGGCCGGCACCAG
CAGCCTCACCGAGTGCGTGCTGAATAAGGCTACCA
ACGTGGCTCACTGGACAACACCCTCTTTAAAGTGC
ATCCGG.

In some embodiments, the first chimeric polypeptide can include a sequence that 15 is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 64)
MKWVTFISLLFLFSSAYSIPPHVQKSVNNDMIVTD
NNGAVKFPQLCKFCDVRFSTCDNQKSCMSNCSITS
ICEKPQEVCVAVWRKNDENITLETVCHDPKLPYHD
FILEDAASPKCIMKEKKKPGETFFMCSCSSDECND
NIIFSEEYNTSNPDGGGGSGGGGSGGGGSIPPHVQ
KSVNNDMIVTDNNGAVKFPQLCKFCDVRFSTCDNQ
KSCMSNCSITSICEKPQEVCVAVWRKNDENITLET
VCHDPKLPYHDFILEDAASPKCIMKEKKKPGETFF
MCSCSSDECNDNIIFSEEYNTSNPDITCPPPMSVE
HADIWVKSYSLYSRERYICNSGFKRKAGTSSLTEC
VLNKATNVAHWTTPSLKCIR.

In some embodiments, a first chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at 30 least 98% identical, at least 99% identical, or 100% identical) to:

(SEQ ID NO: 65)
ATGAAGTGGGTGACCTTCATCAGCCTGCTGTTCCT
GTTCTCCAGCGCCTACTCCATCCCCCCCCATGTGC
AAAAGAGCGTGAACAACGATATGATCGTGACCGAC
AACAACGGCGCCGTGAAGTTTCCCCAGCTCTGCAA
GTTCTGCGATGTCAGGTTCAGCACCTGCGATAATC
AGAAGTCCTGCATGTCCAACTGCAGCATCACCTCC
ATCTGCGAGAAGCCCCAAGAAGTGTGCGTGGCCGT
GTGGCGGAAAAATGACGAGAACATCACCCTGGAGA
CCGTGTGTCACGACCCCAAGCTCCCTTATCACGAC
TTCATTCTGGAGGACGCTGCCTCCCCCAAATGCAT
CATGAAGGAGAAGAAGAAGCCCGGAGAGACCTTCT
TTATGTGTTCCTGTAGCAGCGACGAGTGTAACGAC
AACATCATCTTCAGCGAAGAGTACAACACCAGCAA
CCCTGATGGAGGTGGCGGATCCGGAGGTGGAGGTT
CTGGTGGAGGTGGGAGTATTCCTCCCCACGTGCAG
AAGAGCGTGAATAATGACATGATCGTGACCGATAA
CAATGGCGCCGTGAAATTTCCCCAGCTGTGCAAAT
TCTGCGATGTGAGGTTTTCCACCTGCGACAACCAG
AAGTCCTGTATGAGCAACTGCTCCATCACCTCCAT
CTGTGAGAAGCCTCAGGAGGTGTGCGTGGCTGTCT
GGCGGAAGAATGACGAGAATATCACCCTGGAAACC
GTCTGCCACGATCCCAAGCTGCCCTACCACGATTT
CATCCTGGAAGACGCCGCCAGCCCTAAGTGCATCA
TGAAAGAGAAAAAGAAGCCTGGCGAGACCTTTTTC
ATGTGCTCCTGCAGCAGCGACGAATGCAACGACAA
TATCATCTTTAGCGAGGAATACAATACCAGCAACC
CCGACATTACATGCCCCCCTCCCATGAGCGTGGAG
CACGCCGACATCTGGGTGAAGAGCTATAGCCTCTA
CAGCCGGGAGAGGTATATCTGTAACAGCGGCTTCA
AGAGGAAGGCCGGCACCAGCAGCCTCACCGAGTGC
GTGCTGAATAAGGCTACCAACGTGGCTCACTGGAC
AACACCCTCTTTAAAGTGCATCCGG.

In some embodiments, a second chimeric polypeptide can include a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 66)
IPPHVQKSVNNDMIVTDNNGAVKFPQLCKFCDVRF
STCDNQKSCMSNCSITSICEKPQEVCVAVWRKNDE
NITLETVCHDPKLPYHDFILEDAASPKCIMKEKKK
PGETFFMCSCSSDECNDNIIFSEEYNTSNPDGGGG
SGGGGSGGGGSIPPHVQKSVNNDMIVTDNNGAVKF
PQLCKFCDVRFSTCDNQKSCMSNCSITSICEKPQE
VCVAVWRKNDENITLETVCHDPKLPYHDFILEDAA
SPKCIMKEKKKPGETFFMCSCSSDECNDNIIFSEE
YNTSNPDSGTTNTVAAYNLTWKSTNFKTILEWEPK
PVNQVYTVQISTKSGDWKSKCFYTTDTECDLTDEI
VKDVKQTYLARVFSYPAGNVESTGSAGEPLYENSP
EFTPYLETNLGQPTIQSFEQVGTKVNVTVEDERTL
VRRNNTFLSLRDVFGKDLIYTLYYWKSSSSGKKTA
KTNTNEFLIDVDKGENYCFSVQAVIPSRTVNRKST
DSPVECMGQEKGEFRENWVNVISNLKKIEDLIQSM
HIDATLYTESDVHPSCKVTAMKCFLLELQVISLES
GDASIHDTVENLIILANNSLSSNGNVTESGCKECE
ELEEKNIKEFLQSFVHIVQMFINTS.

In some embodiments, a second chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 67)
ATCCCACCGCACGTTCAGAAGTCGGTGAATAACGA
CATGATAGTCACTGACAACAACGGTGCAGTCAAGT
TTCCACAACTGTGTAAATTTTGTGATGTGAGATTT
TCCACCTGTGACAACCAGAAATCCTGCATGAGCAA
CTGCAGCATCACCTCCATCTGTGAGAAGCCACAGG
AAGTCTGTGTGGCTGTATGGAGAAAGAATGACGAG
AACATAACACTAGAGACAGTTTGCCATGACCCCAA
GCTCCCCTACCATGACTTTATTCTGGAAGATGCTG
CTTCTCCAAAGTGCATTATGAAGGAAAAAAAAAAG
CCTGGTGAGACTTTCTTCATGTGTTCCTGTAGCTC
TGATGAGTGCAATGACAACATCATCTTCTCAGAAG
AATATAACACCAGCAATCCTGACGGAGGTGGCGGA
TCCGGAGGTGGAGGTTCTGGTGGAGGTGGGAGTAT
TCCTCCCCACGTGCAGAAGAGCGTGAATAATGACA
TGATCGTGACCGATAACAATGGCGCCGTGAAATTT
CCCCAGCTGTGCAAATTCTGCGATGTGAGGTTTTC
CACCTGCGACAACCAGAAGTCCTGTATGAGCAACT
GCTCCATCACCTCCATCTGTGAGAAGCCTCAGGAG
GTGTGCGTGGCTGTCTGGCGGAAGAATGACGAGAA
TATCACCCTGGAAACCGTCTGCCACGATCCCAAGC
TGCCCTACCACGATTTCATCCTGGAAGACGCCGCC
AGCCCTAAGTGCATCATGAAAGAGAAAAAGAAGCC
TGGCGAGACCTTTTTCATGTGCTCCTGCAGCAGCG
ACGAATGCAACGACAATATCATCTTTAGCGAGGAA
TACAATACCAGCAACCCCGACTCAGGCACTACAAA
TACTGTGGCAGCATATAATTTAACTTGGAAATCAA
CTAATTTCAAGACAATTTTGGAGTGGGAACCCAAA
CCCGTCAATCAAGTCTACACTGTTCAAATAAGCAC
TAAGTCAGGAGATTGGAAAAGCAAATGCTTTTACA
CAACAGACACAGAGTGTGACCTCACCGACGAGATT
GTGAAGGATGTGAAGCAGACGTACTTGGCACGGGT
CTTCTCCTACCCGGCAGGGAATGTGGAGAGCACCG
GTTCTGCTGGGGAGCCTCTGTATGAGAACTCCCCA
GAGTTCACACCTTACCTGGAGACAAACCTCGGACA
GCCAACAATTCAGAGTTTTGAACAGGTGGGAACAA
AAGTGAATGTGACCGTAGAAGATGAACGGACTTTA
GTCAGAAGGAACAACACTTTCCTAAGCCTCCGGGA
TGTTTTTGGCAAGGACTTAATTTATACACTTTATT
ATTGGAAATCTTCAAGTTCAGGAAAGAAAACAGCC
AAAACAAACACTAATGAGTTTTTGATTGATGTGGA
TAAAGGAGAAAACTACTGTTTCAGTGTTCAAGCAG
TGATTCCCTCCCGAACAGTTAACCGGAAGAGTACA
GACAGCCCGGTAGAGTGTATGGGCCAGGAGAAAGG
GGAATTCAGAGAAAACTGGGTGAATGTAATAAGTA
ATTTGAAAAAAATTGAAGATCTTATTCAATCTATG
CATATTGATGCTACTTTATATACGGAAAGTGATGT
TCACCCCAGTTGCAAAGTAACAGCAATGAAGTGCT
TTCTCTTGGAGTTACAAGTTATTTCACTTGAGTCC
GGAGATGCAAGTATTCATGATACAGTAGAAAATCT
GATCATCCTAGCAAACAACAGTTTGTCTTCTAATG
GGAATGTAACAGAATCTGGATGCAAAGAATGTGAG
GAACTGGAGGAAAAAAATATTAAAGAATTTTTGCA
GAGTTTTGTACATATTGTCCAAATGTTCATCAACA
CTTCT.

In some embodiments, a second chimeric polypeptide can include a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 68)
MGVKVLFALICIAVAEAIPPHVQKSVNNDMIVTDN
NGAVKFPQLCKFCDVRFSTCDNQKSCMSNCSITSI
CEKPQEVCVAVWRKNDENITLETVCHDPKLPYHDF
ILEDAASPKCIMKEKKKPGETFFMCSCSSDECNDN
IIFSEEYNTSNPDGGGGSGGGGSGGGGSIPPHVQK
SVNNDMIVTDNNGAVKFPQLCKFCDVRFSTCDNQK
SCMSNCSITSICEKPQEVCVAVWRKNDENITLETV
CHDPKLPYHDFILEDAASPKCIMKEKKKPGETFFM
CSCSSDECNDNIIFSEEYNTSNPDSGTTNTVAAYN
LTWKSTNFKTILEWEPKPVNQVYTVQISTKSGDWK
SKCFYTTDTECDLTDEIVKDVKQTYLARVFSYPAG
NVESTGSAGEPLYENSPEFTPYLETNLGQPTIQSF
EQVGTKVNVTVEDERTLVRRNNTFLSLRDVFGKDL
IYTLYYWKSSSSGKKTAKTNTNEFLIDVDKGENYC
FSVQAVIPSRTVNRKSTDSPVECMGQEKGEFRENW
VNVISNLKKIEDLIQSMHIDATLYTESDVHPSCKV
TAMKCFLLELQVISLESGDASIHDTVENLIILANN
SLSSNGNVTESGCKECEELEEKNIKEFLQSFVHIV
QMFINTS.

In some embodiments, a second chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 69)
ATGGGAGTGAAAGTTCTTTTTGCCCTTATTTGTAT
TGCTGTGGCCGAGGCCATCCCACCGCACGTTCAGA
AGTCGGTGAATAACGACATGATAGTCACTGACAAC
AACGGTGCAGTCAAGTTTCCACAACTGTGTAAATT
TTGTGATGTGAGATTTTCCACCTGTGACAACCAGA
AATCCTGCATGAGCAACTGCAGCATCACCTCCATC
TGTGAGAAGCCACAGGAAGTCTGTGTGGCTGTATG
GAGAAAGAATGACGAGAACATAACACTAGAGACAG
TTTGCCATGACCCCAAGCTCCCCTACCATGACTTT
ATTCTGGAAGATGCTGCTTCTCCAAAGTGCATTAT
GAAGGAAAAAAAAAAGCCTGGTGAGACTTTCTTCA
TGTGTTCCTGTAGCTCTGATGAGTGCAATGACAAC
ATCATCTTCTCAGAAGAATATAACACCAGCAATCC
TGACGGAGGTGGCGGATCCGGAGGTGGAGGTTCTG
GTGGAGGTGGGAGTATTCCTCCCCACGTGCAGAAG
AGCGTGAATAATGACATGATCGTGACCGATAACAA
TGGCGCCGTGAAATTTCCCCAGCTGTGCAAATTCT
GCGATGTGAGGTTTTCCACCTGCGACAACCAGAAG
TCCTGTATGAGCAACTGCTCCATCACCTCCATCTG
TGAGAAGCCTCAGGAGGTGTGCGTGGCTGTCTGGC
GGAAGAATGACGAGAATATCACCCTGGAAACCGTC
TGCCACGATCCCAAGCTGCCCTACCACGATTTCAT
CCTGGAAGACGCCGCCAGCCCTAAGTGCATCATGA
AAGAGAAAAAGAAGCCTGGCGAGACCTTTTTCATG
TGCTCCTGCAGCAGCGACGAATGCAACGACAATAT
CATCTTTAGCGAGGAATACAATACCAGCAACCCCG
ACTCAGGCACTACAAATACTGTGGCAGCATATAAT
TTAACTTGGAAATCAACTAATTTCAAGACAATTTT
GGAGTGGGAACCCAAACCCGTCAATCAAGTCTACA
CTGTTCAAATAAGCACTAAGTCAGGAGATTGGAAA
AGCAAATGCTTTTACACAACAGACACAGAGTGTGA
CCTCACCGACGAGATTGTGAAGGATGTGAAGCAGA
CGTACTTGGCACGGGTCTTCTCCTACCCGGCAGGG
AATGTGGAGAGCACCGGTTCTGCTGGGGAGCCTCT
GTATGAGAACTCCCCAGAGTTCACACCTTACCTGG
AGACAAACCTCGGACAGCCAACAATTCAGAGTTTT
GAACAGGTGGGAACAAAAGTGAATGTGACCGTAGA
AGATGAACGGACTTTAGTCAGAAGGAACAACACTT
TCCTAAGCCTCCGGGATGTTTTTGGCAAGGACTTA
ATTTATACACTTTATTATTGGAAATCTTCAAGTTC
AGGAAAGAAAACAGCCAAAACAAACACTAATGAGT
TTTTGATTGATGTGGATAAAGGAGAAAACTACTGT
TTCAGTGTTCAAGCAGTGATTCCCTCCCGAACAGT
TAACCGGAAGAGTACAGACAGCCCGGTAGAGTGTA
TGGGCCAGGAGAAAGGGGAATTCAGAGAAAACTGG
GTGAATGTAATAAGTAATTTGAAAAAAATTGAAGA
TCTTATTCAATCTATGCATATTGATGCTACTTTAT
ATACGGAAAGTGATGTTCACCCCAGTTGCAAAGTA
ACAGCAATGAAGTGCTTTCTCTTGGAGTTACAAGT
TATTTCACTTGAGTCCGGAGATGCAAGTATTCATG
ATACAGTAGAAAATCTGATCATCCTAGCAAACAAC
AGTTTGTCTTCTAATGGGAATGTAACAGAATCTGG
ATGCAAAGAATGTGAGGAACTGGAGGAAAAAAATA
TTAAAGAATTTTTGCAGAGTTTTGTACATATTGTC
CAAATGTTCATCAACACTTCT.

### Compositions/Kits

Also provided herein are compositions (e.g., pharmaceutical compositions) that include at least one of any multi-chain chimeric polypeptides, any of the cells, or any of the nucleic acids described herein. In some embodiments, the compositions include at least one of any of the multi-chain chimeric polypeptides described herein. In some embodiments, the compositions include any of the immune cells (e.g., any of the immune cells described herein, e.g., any of the immune cells produced using any of the methods described herein).

In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

Single or multiple administrations of pharmaceutical compositions can be given to a subject in need thereof depending on for example: the dosage and frequency as required and tolerated by the subject. The formulation should provide a sufficient quantity of active agent to effectively treat, prevent or ameliorate conditions, diseases or symptoms.

Also provided herein are kits that include any of the multi-chain chimeric polypeptides, compositions, nucleic acids, or cells (e.g., immune cells) described herein. In some embodiments, the kits can include instructions for performing any of the methods described herein. In some embodiments, the kits can include at least one dose of any of the pharmaceutical compositions described herein.

### Nucleic Acids/Vectors

Also provided herein are nucleic acids that encode any of the multi-chain chimeric polypeptides described herein. In some embodiments, a first nucleic acid can encode the first chimeric polypeptide and a second nucleic acid can encode the second chimeric polypeptide. In some embodiments, a single nucleic acid can encode both the first chimeric polypeptide and the second chimeric polypeptide.

Also provided herein are vectors that include any of the nucleic acids encoding any of the multi-chain chimeric polypeptides described herein. In some embodiments, a first vector can include a nucleic acid encoding the first chimeric polypeptide and a second vector can include a nucleic acid encoding the second chimeric polypeptide. In some embodiments, a single vector can include a first nucleic acid encoding the first chimeric polypeptide and a second nucleic acid encoding the second chimeric polypeptide.

Any of the vectors described herein can be an expression vector. For example, an expression vector can include a promoter sequence operably linked to the sequence encoding the first chimeric polypeptide and the second chimeric polypeptide.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, e.g., include sufficient cis-acting elements for expression; other elements for expression can be supplied by the host mammalian cell or in an in vitro expression system. Skilled practitioners will be capable of selecting suitable vectors and mammalian cells for making any of the multi-chain chimeric polypeptides described herein.

### Cells

Also provided herein are cells (e.g., any of the exemplary cells described herein or known in the art) comprising any of the nucleic acids described herein that encode any of the multi-chain chimeric polypeptides described herein (e.g., encoding both the first and second chimeric polypeptides). Also provided herein are cells (e.g., any of the exemplary cells described herein or known in the art) comprising any of the nucleic acids described herein that encode any of the first chimeric polypeptides described herein. Also provided are cells (e.g., any of the exemplary cells described herein or known in the art) comprising any of the nucleic acids described herein that encode any of the second chimeric polypeptides described herein.

Also provided herein are cells (e.g., any of the exemplary cells described herein or known in the art) that include any of the vectors described herein that encode any of the multi-chain chimeric polypeptides described herein (e.g., encoding both the first and second chimeric polypeptides). Also provided herein are cells (e.g., any of the exemplary cells described herein or known in the art) that include any of the vectors described herein that encode any of the first chimeric polypeptides described herein. Also provided herein are cells (e.g., any of the exemplary cells described herein or known in the art) that include any of the vectors described herein that encode any of the second chimeric polypeptides described herein).

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

### Methods of Producing Multi-Chain Chimeric Polypeptides

Also provided herein are methods of producing any of the multi-chain chimeric polypeptides described herein that include culturing any of the cells described herein in a culture medium under conditions sufficient to result in the production of the multi-chain chimeric polypeptide; and recovering the multi-chain chimeric polypeptide from the cell and/or the culture medium.

Also provided herein are method of producing any of the multi-chain chimeric polypeptides described herein that include: culturing any of cells described herein in a first culture medium under conditions sufficient to result in the production of the first chimeric polypeptide; recovering the first chimeric polypeptide from the cell and/or the first culture medium; culturing any of the cells described herein in a second culture medium under conditions sufficient to result in the production of the second chimeric polypeptide; recovering the second chimeric polypeptide from the cell and/or the second culture medium; and combining (e.g., mixing) the recovered first chimeric polypeptide and the recovered second chimeric polypeptide to form the multi-chain chimeric polypeptide (e.g., any of the multi-chain chimeric polypeptides described herein).

The recovery of the multi-chain chimeric polypeptide, the first chimeric polypeptide, or the second chimeric polypeptide from a cell (e.g., a eukaryotic cell) can be performed using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

Methods of culturing cells are well known in the art. Cells can be maintained in vitro under conditions that favor proliferation, differentiation and growth. Briefly, cells can be cultured by contacting a cell (e.g., any cell) with a cell culture medium that includes the necessary growth factors and supplements to support cell viability and growth.

Also provided herein are multi-chain chimeric polypeptides (e.g., any of the multi-chain chimeric polypeptides described herein), first chimeric polypeptides (e.g., any of the first chimeric polypeptides), or second chimeric polypeptides (e.g., any of the second chimeric polypeptides described herein) produced by any of the methods described herein.

### Senescent Cells

Senescence is a form of irreversible growth arrest accompanied by phenotypic changes, resistance to apoptosis and activation of damage-sensing signaling pathways. Cellular senescence was first described in cultured human fibroblast cells that lost their ability to proliferate, reaching permanent arrest after about 50 population doublings (referred to as the Hayflick limit). Senescence is considered a stress response that can be induced by a wide range of intrinsic and extrinsic insults, including oxidative and genotoxic stress, DNA damage, telomere attrition, oncogenic activation, mitochondrial dysfunction, or chemotherapeutic agents.

Senescent cells remain metabolically active and can influence the tissue hemostasis, disease and aging through their secretory phenotype. Senescence is considered as a physiologic process and is important in promoting wound healing, tissue homeostasis, regeneration, and fibrosis regulation. For instance, transient induction of senescent cells is observed during would healing and contributes to wound resolution. Perhaps one of the most important roles of senescence is its role in tumor suppression. However, the accumulation of senescent cells also drives aging- and aging-related diseases and conditions. The senescent phenotype also can trigger chronic inflammatory responses and consequently augment chronic inflammatory conditions to promote tumor growth. The connection between senescence and aging was initially based on observations that senescent cells accumulate in aged tissue. The use of transgenic models has enabled the detection of senescent cells systematically in many age-related pathologies. Strategies to selectively eliminate senescent cells has demonstrated that senescent cells can indeed play a causal role in aging and related pathologies.

Senescent cells display important and unique properties which include changes in morphology, chromatin organization, gene expression, and metabolism. There are several biochemical and functional properties associated with cellular senescence, such as (i) increased expression of p16 and p21, inhibitors of cyclin-dependent kinases, (ii) presence of senescence-associated β-galactosidase, a marker of lysosomal activity, (iii) appearance of senescence-associated heterochromatin foci and downregulation of lamin B1 levels, (iv) resistance to apoptosis caused by an increased expression of anti-apoptotic BCL-family protein, and (v) upregulation of CD26 (DPP4), CD36 (Scavenger receptor), forkhead box 4 (FOXO4), and secretory carrier membrane protein 4 (SCAMP4). Senescent cells also express an inflammatory signature, the so-called senescence-associated secretory phenotype (SASP). Through SASP, the senescent cells produce a wide range of inflammatory cytokines (IL-6, IL-8), growth factors (TGF-β, chemokines (CCL-2), and matrix metalloproteinases (MMP-3, MMP-9) that operate in a cell-autonomous manner to reinforce senescence (autocrine effects) and communicate with and modify the microenvironment (paracrine effects). SASP factors can contribute to tumor suppression by triggering senescence surveillance, an immune-mediated clearance of senescent cells. However, chronic inflammation is also a known driver of tumorigenesis, and accumulating evidence indicates that chronic SASP can also boost cancer and aging-related diseases.

The secretion profile of senescent cells is context dependent. For instance, the mitochondrial dysfunction-associated senescence (MiDAS), induced by different mitochondrial dysfunction in human fibroblasts, led to the appearance of a SASP that was deficient in IL-1-dependent inflammatory factors. A decrease in the NAD+/NADH ratio activated AMPK signaling which induced MiDAS through the activation of p53. As a result, p53 inhibited NF-KB signaling which is a crucial inducer of pro-inflammatory SASP. In contrast, the cellular senescence caused by persistent DNA damage in human cells induced an inflammatory SASP, which was dependent on the activation of ataxia-telangiectasia mutated (ATM) kinase but not on that of p53. In particular, the expression and secretion levels of IL-6 and IL-8 were increased. It was also demonstrated that cellular senescence caused by the ectopic expression p16INK4a and p21CIP1 induced the senescent phenotype in human fibroblasts without an inflammatory SASP indicating that the growth arrest itself did not stimulate SASP.

One of the most defining characteristics of senescence is stable growth arrest. This is achieved by two important pathways, the p16/Rb and the p53/p21, both of which are central in tumor suppression. DNA damage results in: (1) high deposition of yH2Ax (histone coding gene) and 53BP1 (involved in DNA damage response) in chromatin: this leads to activation of a kinase cascade eventually resulting in p53 activation, and (2) activation of pl6INK4a and ARF (both encoded by CDKN2A) and P15INK4b (encoded by CDKN2B): p53 induces transcription of cyclin-dependent kinase inhibitor (p21) and along with both pl6INK4a and pl5INK4b block genes for cell cycle progression (CDK4 and CDK6). This eventually leads to hypophosphorylation of Retinoblastoma protein (Rb) and cell cycle arrest at the G1 phase.

Selectively killing senescent cells has been shown to significantly improve the health span of mice in the context of normal aging and ameliorates the consequences of age-related disease or cancer therapy (Ovadya, J Clin Invest. 128(4):1247-1254, 2018). In nature, the senescent cells are normally removed by the innate immune cells. Induction of senescence not only prevents the potential proliferation and transformation of damaged/altered cells, but also favors tissue repair through the production of SASP factors that function as chemoattractants mainly for Natural Killer (NK) cells (such as IL-15 and CCL2) and macrophages (such as CFS-1 and CCL2). These innate immune cells mediate the immunosurveillance mechanism for eliminating stressed cells. Senescent cells usually up-regulate the NK-cell activating receptor NKG2D and DNAM-1 ligands, which belong to a family of stress-inducible ligands: an important component of the frontline immune defense against infectious diseases and malignancies. Upon receptor activation, NK cells can then specifically induce the death of senescent cells through their cytolytic machinery. A role for NK cells in the immune surveillance of senescent cells has been pointed out in liver fibrosis (Sagiv, Oncogene 32(15): 1971-1977, 2013), hepatocellular carcinoma (Iannello, J Exp Med 210(10): 2057-2069, 2013), multiple myeloma (Soriani, Blood 113(15): 3503-3511, 2009), and glioma cells stressed by dysfunction of the mevalonate pathway (Ciaglia, Int J Cancer 142(1): 176-190, 2018). Endometrial cells undergo acute cellular senescence and do not differentiate into decidual cells. The differentiated decidual cells secrete IL-15 and thereby recruit uterine NK cells to target and eliminate the undifferentiated senescent cells thus helping to re-model and rejuvenate the endometrium (Brighton, Elife 6: e31274, 2017). With a similar mechanism, during liver fibrosis, p53-expressing senescent liver satellite cells skewed the polarization of resident Kupfer macrophages and freshly infiltrated macrophages toward the pro-inflammatory M1 phenotype, which display senolytic activity. F4/80+ macrophages have been shown to play a key role in the clearance of mouse uterine senescent cells to maintain postpartum uterine function.

Senescent cells recruit NK cells by mainly upregulating ligands to NKG2D (expressed on NK cells), chemokines, and other SASP factors. In vivo models of liver fibrosis have shown effective clearance of senescent cells by activated NK cells (Krizhanovsky, Cell 134(4): 657-667, 2008). Studies have described various models to study senescence including liver fibrosis (Krizhanovsky, Cell 134(4): 657-667, 2008), osteoarthritis (Xu, J Gerontol A Blot Sci Med Sci 72(6): 780-785, 2017), and Parkinson's disease (Chinta, Cell Rep 22(4): 930-940, 2018). Animal models for studying senescent cells are described in: Krizhanovsky, Cell 134(4): 657-667, 2008; Baker, Nature 479(7372): 232-236, 2011; Farr, Nat Med 23(9): 1072-1079, 2017; Bourgeois, FEBS Lett 592(12): 2083-2097, 2018; Xu, Nat Med 24(8): 1246-1256, 2018).

### Methods of Treating a Liver Disease or a Metabolic Syndrome in a Subject

Also provided herein are methods of treating a liver disease or a metabolic syndrome in a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGT-βRII) and the second target-binding domain binds specifically to a ligand of TGF-βRII (e.g., any of the exemplary multi-chain chimeric polypeptides described herein). In some embodiments, the subject is diagnosed or identified as having the liver disease or the metabolic syndrome.

In some embodiments, the multi-chain chimeric polypeptide is administered via intramuscular administration, subcutaneous administration, intravenous administration, intrahepatic administration, or intraperitoneal administration.

In some embodiments, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments, the metabolic syndrome is selected from the group of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts. Methods for accessing successful treatment of the liver diseases and metabolic syndromes are known in the art.

In some embodiments, the subject has not been previously identified or diagnosed as having type 2 diabetes mellitus. In some embodiments, the subject has not been previously identified or diagnosed as having adipose atrophy. In some embodiments, the subject has not been previously identified or diagnosed as having lipodystrophy. In some embodiments, the subject has not been previously identified or diagnosed as having liver cirrhosis. In some embodiments, the subject has not been previously identified or diagnosed as having NAFLD. In some embodiments, the subject has not been previously identified or diagnosed as having non-alcoholic steatohepatitis.

Methods of Reducing One or More of the Rate of Progression from NAFL to NASH, Rate of Progression from NASH to Cirrhosis, and Rate of Progression from Cirrhosis to Hepatocellular Carcinoma

Also provided are methods of reducing one or more of the rate of: progression from non-alcoholic fatty liver disease (NAFL) to non-alcoholic steatohepatitis (NASH), progression from NASH to cirrhosis, and progression from cirrhosis to hepatocellular carcinoma, that include administering to a subject identified or diagnosed as having NAFL, NASH, or cirrhosis, a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGT-βRII) and the second target-binding domain binds specifically to a ligand of TGF-βRII (e.g., any of the exemplary multi-chain chimeric polypeptides described herein).

In some embodiments, the multi-chain chimeric polypeptide is administered via intramuscular administration, subcutaneous administration, intravenous administration, intrahepatic administration, or intraperitoneal administration.

In some embodiments, the method results in a decrease (e.g.., about a 1% decrease to about a 100% decrease, about a 1% decrease to about a 95% decrease, about a 1% decrease to about a 90% decrease, about a 1% decrease to about a 85% decrease, about a 1% decrease to about a 80% decrease, about a 1% decrease to about a 75% decrease, about a 1% decrease to about a 70% decrease, about a 1% decrease to about a 65% decrease, about a 1% decrease to about a 60% decrease, about a 1% decrease to about a 55% decrease, about a 1% decrease to about a 50% decrease, about a 1% decrease to about a 45% decrease, about a 1% decrease to about a 40% decrease, about a 1% decrease to about a 35% decrease, about a 1% decrease to about a 30% decrease, about a 1% decrease to about a 25% decrease, about a 1% decrease to about a 20% decrease, about a 1% decrease to about a 15% decrease, about a 1% decrease to about a 10% decrease, about a 1% decrease to about a 5% decrease, about a 5% decrease to about a 100% decrease, about a 5% decrease to about a 95% decrease, about a 5% decrease to about a 90% decrease, about a 5% decrease to about a 85% decrease, about a 5% decrease to about a 80% decrease, about a 5% decrease to about a 75% decrease, about a 5% decrease to about a 70% decrease, about a 5% decrease to about a 65% decrease, about a 5% decrease to about a 60% decrease, about a 5% decrease to about a 55% decrease, about a 5% decrease to about a 50% decrease, about a 5% decrease to about a 45% decrease, about a 5% decrease to about a 40% decrease, about a 5% decrease to about a 35% decrease, about a 5% decrease to about a 30% decrease, about a 5% decrease to about a 25% decrease, about a 5% decrease to about a 20% decrease, about a 5% decrease to about a 15% decrease, about a 5% decrease to about a 10% decrease, about a 10% decrease to about a 100% decrease, about a 10% decrease to about a 95% decrease, about a 10% decrease to about a 90% decrease, about a 10% decrease to about a 85% decrease, about a 10% decrease to about a 80% decrease, about a 10% decrease to about a 75% decrease, about a 10% decrease to about a 70% decrease, about a 10% decrease to about a 65% decrease, about a 10% decrease to about a 60% decrease, about a 10% decrease to about a 55% decrease, about a 10% decrease to about a 50% decrease, about a 10% decrease to about a 45% decrease, about a 10% decrease to about a 40% decrease, about a 10% decrease to about a 35% decrease, about a 10% decrease to about a 30% decrease, about a 10% decrease to about a 25% decrease, about a 10% decrease to about a 20% decrease, about a 10% decrease to about a 15% decrease, about a 15% decrease to about a 100% decrease, about a 15% decrease to about a 95% decrease, about a 15% decrease to about a 90% decrease, about a 15% decrease to about a 85% decrease, about a 15% decrease to about a 80% decrease, about a 15% decrease to about a 75% decrease, about a 15% decrease to about a 70% decrease, about a 15% decrease to about a 65% decrease, about a 15% decrease to about a 60% decrease, about a 15% decrease to about a 55% decrease, about a 15% decrease to about a 50% decrease, about a 15% decrease to about a 45% decrease, about a 15% decrease to about a 40% decrease, about a 15% decrease to about a 35% decrease, about a 15% decrease to about a 30% decrease, about a 15% decrease to about a 25% decrease, about a 15% decrease to about a 20% decrease, about a 20% decrease to about a 100% decrease, about a 20% decrease to about a 95% decrease, about a 20% decrease to about a 90% decrease, about a 20% decrease to about a 85% decrease, about a 20% decrease to about a 80% decrease, about a 20% decrease to about a 75% decrease, about a 20% decrease to about a 70% decrease, about a 20% decrease to about a 65% decrease, about a 20% decrease to about a 60% decrease, about a 20% decrease to about a 55% decrease, about a 20% decrease to about a 50% decrease, about a 20% decrease to about a 45% decrease, about a 20% decrease to about a 40% decrease, about a 20% decrease to about a 35% decrease, about a 20% decrease to about a 30% decrease, about a 20% decrease to about a 25% decrease, about a 25% decrease to about a 100% decrease, about a 25% decrease to about a 95% decrease, about a 25% decrease to about a 90% decrease, about a 25% decrease to about a 85% decrease, about a 25% decrease to about a 80% decrease, about a 25% decrease to about a 75% decrease, about a 25% decrease to about a 70% decrease, about a 25% decrease to about a 65% decrease, about a 25% decrease to about a 60% decrease, about a 25% decrease to about a 55% decrease, about a 25% decrease to about a 50% decrease, about a 25% decrease to about a 45% decrease, about a 25% decrease to about a 40% decrease, about a 25% decrease to about a 35% decrease, about a 25% decrease to about a 30% decrease, about a 30% decrease to about a 100% decrease, about a 30% decrease to about a 95% decrease, about a 30% decrease to about a 90% decrease, about a 30% decrease to about a 85% decrease, about a 30% decrease to about a 80% decrease, about a 30% decrease to about a 75% decrease, about a 30% decrease to about a 70% decrease, about a 30% decrease to about a 65% decrease, about a 30% decrease to about a 60% decrease, about a 30% decrease to about a 55% decrease, about a 30% decrease to about a 50% decrease, about a 30% decrease to about a 45% decrease, about a 30% decrease to about a 40% decrease, about a 30% decrease to about a 35% decrease, about a 35% decrease to about a 100% decrease, about a 35% decrease to about a 95% decrease, about a 35% decrease to about a 90% decrease, about a 35% decrease to about a 85% decrease, about a 35% decrease to about a 80% decrease, about a 35% decrease to about a 75% decrease, about a 35% decrease to about a 70% decrease, about a 35% decrease to about a 65% decrease, about a 35% decrease to about a 60% decrease, about a 35% decrease to about a 55% decrease, about a 35% decrease to about a 50% decrease, about a 35% decrease to about a 45% decrease, about a 35% decrease to about a 40% decrease, about a 40% decrease to about a 100% decrease, about a 40% decrease to about a 95% decrease, about a 40% decrease to about a 90% decrease, about a 40% decrease to about a 85% decrease, about a 40% decrease to about a 80% decrease, about a 40% decrease to about a 75% decrease, about a 40% decrease to about a 70% decrease, about a 40% decrease to about a 65% decrease, about a 40% decrease to about a 60% decrease, about a 40% decrease to about a 55% decrease, about a 40% decrease to about a 50% decrease, about a 40% decrease to about a 45% decrease, about a 45% decrease to about a 100% decrease, about a 45% decrease to about a 95% decrease, about a 45% decrease to about a 90% decrease, about a 45% decrease to about a 85% decrease, about a 45% decrease to about a 80% decrease, about a 45% decrease to about a 75% decrease, about a 45% decrease to about a 70% decrease, about a 45% decrease to about a 65% decrease, about a 45% decrease to about a 60% decrease, about a 45% decrease to about a 55% decrease, about a 45% decrease to about a 50% decrease, about a 50% decrease to about a 100% decrease, about a 50% decrease to about a 95% decrease, about a 50% decrease to about a 90% decrease, about a 50% decrease to about a 85% decrease, about a 50% decrease to about a 80% decrease, about a 50% decrease to about a 75% decrease, about a 50% decrease to about a 70% decrease, about a 50% decrease to about a 65% decrease, about a 50% decrease to about a 60% decrease, about a 50% decrease to about a 55% decrease, about a 55% decrease to about a 100% decrease, about a 55% decrease to about a 95% decrease, about a 55% decrease to about a 90% decrease, about a 55% decrease to about a 85% decrease, about a 55% decrease to about a 80% decrease, about a 55% decrease to about a 75% decrease, about a 55% decrease to about a 70% decrease, about a 55% decrease to about a 65% decrease, about a 55% decrease to about a 60% decrease, about a 60% decrease to about a 100% decrease, about a 60% decrease to about a 95% decrease, about a 60% decrease to about a 90% decrease, about a 60% decrease to about a 85% decrease, about a 60% decrease to about a 80% decrease, about a 60% decrease to about a 75% decrease, about a 60% decrease to about a 70% decrease, about a 60% decrease to about a 65% decrease, about a 65% decrease to about a 100% decrease, about a 65% decrease to about a 95% decrease, about a 65% decrease to about a 90% decrease, about a 65% decrease to about a 85% decrease, about a 65% decrease to about a 80% decrease, about a 65% decrease to about a 75% decrease, about a 65% decrease to about a 70% decrease, about a 70% decrease to about a 100% decrease, about a 70% decrease to about a 95% decrease, about a 70% decrease to about a 90% decrease, about a 70% decrease to about a 85% decrease, about a 70% decrease to about a 80% decrease, about a 70% decrease to about a 75% decrease, about a 75% decrease to about a 100% decrease, about a 75% decrease to about a 95% decrease, about a 75% decrease to about a 90% decrease, about a 75% decrease to about a 85% decrease, about a 75% decrease to about a 80% decrease, about a 80% decrease to about a 100% decrease, about a 80% decrease to about a 95% decrease, about a 80% decrease to about a 90% decrease, about a 80% decrease to about a 85% decrease, about a 85% decrease to about a 100% decrease, about a 85% decrease to about a 95% decrease, about a 85% decrease to about a 90% decrease, about a 90% decrease to about a 100% decrease, about a 90% decrease to about a 95% decrease, about a 95% decrease to about a 100% decrease, or about a 95% to about a 99% decrease) in the rate of progression from NAFL to NASH, e.g., as compared to the rate of progression before treatment or the rate of progression in a similar subject identified as having NAFL and receiving no treatment or a different treatment.

In some embodiments, the method results in a decrease (e.g., about a 1% decrease to about a 100% decrease, or any of the subranges of this range described herein) in the rate of progression from NASH to cirrhosis, e.g., as compared to the rate of progression before treatment or the rate of progression in a similar subject identified as having NASH and receiving no treatment or a different treatment.

In some embodiments, the method results in a decrease (e.g., about a 1% decrease to about a 100% decrease, or any of the subranges of this range described herein) in the rate of progression from cirrhosis to hepatocellular carcinoma, e.g., e.g., as compared to the rate of progression before treatment or the rate of progression in a similar subject identified as having cirrhosis and receiving no treatment or a different treatment.

### Methods of Reducing Inflammation in a Liver of a Subject

Also provided herein are methods of reducing inflammation in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGT-βRII) and the second target-binding domain binds specifically to a ligand of TGF-βRII (e.g., any of the exemplary multi-chain chimeric polypeptides described herein). In some embodiments, the subject is identified as being in need of a reduction in inflammation in their liver. Methods for determining a level of inflammation in the liver of a subject are known in the art and include, e.g., detecting the level of expression of one or more inflammatory cytokines in the liver of the subject.

In some embodiments, the multi-chain chimeric polypeptide is administered via intramuscular administration, subcutaneous administration, intravenous administration, intrahepatic administration, or intraperitoneal administration.

In some embodiments, the method results in a decrease (e.g., about a 1% decrease to about a 100% decrease, or any of the subranges of this range described herein) in the level of inflammation in the liver of a subject (e.g., any of the subjects described herein), e.g., as compared to the level of inflammation in the liver of the subject prior to the administering.

In some embodiments, the subject has been previously identified or diagnosed as having a liver disease (e.g., any of the exemplary liver diseases described herein or known in the art) or a metabolic syndrome (e.g., any of the exemplary metabolic syndromes described herein or known in the art). In some embodiments, the subject has been previously identified or diagnosed as having a liver disease (e.g., any of the exemplary liver diseases described herein or known in the art). In some embodiments, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments, the subject has been previously identified or diagnosed as having a metabolic syndrome (e.g., any of the exemplary metabolic syndromes described herein or known in the art). In some embodiments, the metabolic syndrome is selected from the group of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

In some embodiments, the subject has not been previously identified or diagnosed as having type 2 diabetes mellitus. In some embodiments, the subject has not been previously identified or diagnosed as having adipose atrophy. In some embodiments, the subject has not been previously identified or diagnosed as having lipodystrophy. In some embodiments, the subject has not been previously identified or diagnosed as having liver cirrhosis. In some embodiments, the subject has not been previously identified or diagnosed as having NAFLD. In some embodiments, the subject has not been previously identified or diagnosed as having non-alcoholic steatohepatitis.

### Methods of Decreasing Gluconeogenesis in a Liver of a Subject

Also provided herein are methods of decreasing gluconeogenesis in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and

(b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGT-βRII) and the second target-binding domain binds specifically to a ligand of TGF-βRII (e.g., any of the exemplary multi-chain chimeric polypeptides described herein). In some embodiments, the subject is identified as being in need of a decrease in gluconeogenesis in their liver. Methods of detecting the level of gluconeogenesis in a liver of a subject are known in the art.

In some embodiments, the multi-chain chimeric polypeptide is administered via intramuscular administration, subcutaneous administration, intravenous administration, intrahepatic administration, or intraperitoneal administration.

In some embodiments, the method results in a decrease (e.g., about a 1% decrease to about a 100% decrease, or any of the subranges of this range described herein) in the level of gluconeogenesis in the liver of a subject (e.g., any of the subjects described herein), e.g., as compared to the level of gluconeogenesis in the liver of the subject prior to the administering.

In some embodiments, the subject has been previously identified or diagnosed as having a liver disease (e.g., any of the exemplary liver diseases described herein or known in the art) or a metabolic syndrome (e.g., any of the exemplary metabolic syndromes described herein or known in the art). In some embodiments, the subject has been previously identified or diagnosed as having a liver disease (e.g., any of the exemplary liver diseases described herein or known in the art). In some embodiments, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments, the subject has been previously identified or diagnosed as having a metabolic syndrome (e.g., any of the exemplary metabolic syndromes described herein or known in the art). In some embodiments, the metabolic syndrome is selected from the group of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

In some embodiments, the subject has not been previously identified or diagnosed as having type 2 diabetes mellitus. In some embodiments, the subject has not been previously identified or diagnosed as having adipose atrophy. In some embodiments, the subject has not been previously identified or diagnosed as having lipodystrophy. In some embodiments, the subject has not been previously identified or diagnosed as having liver cirrhosis. In some embodiments, the subject has not been previously identified or diagnosed as having NAFLD. In some embodiments, the subject has not been previously identified or diagnosed as having non-alcoholic steatohepatitis.

### Methods of Decreasing Lipogenesis in a Liver of a Subject

Also provided herein are methods of decreasing lipogenesis in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII (e.g., any of the exemplary multi-chain chimeric polypeptides described herein). In some embodiments, the subject is identified as being in need of a decrease in lipogenesis in their liver. Methods for detecting the level of lipogenesis in a liver of a subject are known in the art.

In some embodiments, the multi-chain chimeric polypeptide is administered via intramuscular administration, subcutaneous administration, intravenous administration, intrahepatic administration, or intraperitoneal administration.

In some embodiments, the method results in a decrease (e.g., about a 1% decrease to about a 100% decrease, or any of the subranges of this range described herein) in the level of lipogenesis in the liver of a subject (e.g., any of the subjects described herein), e.g., as compared to the level of lipogenesis in the liver of the subject prior to the administering.

In some embodiments, the subject has been previously identified or diagnosed as having a liver disease (e.g., any of the exemplary liver diseases described herein or known in the art) or a metabolic syndrome (e.g., any of the exemplary metabolic syndromes described herein or known in the art). In some embodiments, the subject has been previously identified or diagnosed as having a liver disease (e.g., any of the exemplary liver diseases described herein or known in the art). In some embodiments, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments, the subject has been previously identified or diagnosed as having a metabolic syndrome (e.g., any of the exemplary metabolic syndromes described herein or known in the art). In some embodiments, the metabolic syndrome is selected from the group of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

In some embodiments, the subject has not been previously identified or diagnosed as having type 2 diabetes mellitus. In some embodiments, the subject has not been previously identified or diagnosed as having adipose atrophy. In some embodiments, the subject has not been previously identified or diagnosed as having lipodystrophy. In some embodiments, the subject has not been previously identified or diagnosed as having liver cirrhosis. In some embodiments, the subject has not been previously identified or diagnosed as having NAFLD. In some embodiments, the subject has not been previously identified or diagnosed as having non-alcoholic steatohepatitis.

### Methods of Decreasing Hepatocytic Senescence in a Liver of a Subject

Also provided herein are methods of decreasing hepatocytic senescence in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGT-βRII) and the second target-binding domain binds specifically to a ligand of TGF-βRII (e.g., any of the exemplary multi-chain chimeric polypeptides described herein). In some embodiments, the subject is identified as being in need of decreased hepatocytic senescence in their liver. Methods for determining a level of hepatocytic senescence in a liver of a subject are known in the art. In some embodiments, the multi-chain chimeric polypeptide is administered via intramuscular administration, subcutaneous administration, intravenous administration, intrahepatic administration, or intraperitoneal administration.

In some embodiments, the method results in a decrease (e.g., about a 1% decrease to about a 100% decrease, or any of the subranges of this range described herein) in the level of hepatocytic senescence in the liver of a subject (e.g., any of the subjects described herein), e.g., as compared to the level of hepatocytic senescence in the liver of the subject prior to the administering.

In some embodiments, the subject has been previously identified or diagnosed as having a liver disease (e.g., any of the exemplary liver diseases described herein or known in the art) or a metabolic syndrome (e.g., any of the exemplary metabolic syndromes described herein or known in the art). In some embodiments, the subject has been previously identified or diagnosed as having a liver disease (e.g., any of the exemplary liver diseases described herein or known in the art). In some embodiments, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments, the subject has been previously identified or diagnosed as having a metabolic syndrome (e.g., any of the exemplary metabolic syndromes described herein or known in the art). In some embodiments, the metabolic syndrome is selected from the group of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

In some embodiments, the subject has not been previously identified or diagnosed as having type 2 diabetes mellitus. In some embodiments, the subject has not been previously identified or diagnosed as having adipose atrophy. In some embodiments, the subject has not been previously identified or diagnosed as having lipodystrophy. In some embodiments, the subject has not been previously identified or diagnosed as having liver cirrhosis. In some embodiments, the subject has not been previously identified or diagnosed as having NAFLD. In some embodiments, the subject has not been previously identified or diagnosed as having non-alcoholic steatohepatitis.

### Methods of Rebalancing Metabolic Function in a Liver of a Subject

Also provided herein are methods of rebalancing metabolic function in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGT-βRII) and the second target-binding domain binds specifically to a ligand of TGF-βRII (e.g., any of the exemplary multi-chain chimeric polypeptides described herein). In some embodiments, the subject is identified as being in need of rebalancing of metabolic function in their liver. Methods for determining the rebalancing of metabolic function in a liver in the subject are known in the art. Non-limiting embodiments of rebalancing of metabolic function include normalizing blood glucose levels (e.g., hemoglobin Al c levels or fasting glucose levels in a subject), reducing insulin resistance, and normalizing gene expression of Retn (Resistin).

In some embodiments, the multi-chain chimeric polypeptide is administered via intramuscular administration, subcutaneous administration, intravenous administration, intrahepatic administration, or intraperitoneal administration.

In some embodiments, the subject has been previously identified or diagnosed as having a liver disease (e.g., any of the exemplary liver diseases described herein or known in the art) or a metabolic syndrome (e.g., any of the exemplary metabolic syndromes described herein or known in the art). In some embodiments, the subject has been previously identified or diagnosed as having a liver disease (e.g., any of the exemplary liver diseases described herein or known in the art). In some embodiments, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments, the subject has been previously identified or diagnosed as having a metabolic syndrome (e.g., any of the exemplary metabolic syndromes described herein or known in the art). In some embodiments, the metabolic syndrome is selected from the group of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

In some embodiments, the subject has not been previously identified or diagnosed as having type 2 diabetes mellitus. In some embodiments, the subject has not been previously identified or diagnosed as having adipose atrophy. In some embodiments, the subject has not been previously identified or diagnosed as having lipodystrophy. In some embodiments, the subject has not been previously identified or diagnosed as having liver cirrhosis. In some embodiments, the subject has not been previously identified or diagnosed as having NAFLD. In some embodiments, the subject has not been previously identified or diagnosed as having non-alcoholic steatohepatitis.

### Methods of Modulating Expression of One or More Genes in Tables 1-4 in a Liver of a Subject

Also provided herein are methods of modulating expression of one or more (e.g., two or more, three or more, four or more, five or more, six or more, seven or more, eight or more, nine or more, ten or more, fifteen or more, twenty or more, or thirty or more) genes in Tables 1-4 in a liver of a subject that include administering to the subject a therapeutically effective amount of a multi-chain chimeric polypeptide comprising: (a) a first chimeric polypeptide comprising: (i) a first target-binding domain; (ii) soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII (e.g., any of the exemplary multi-chain chimeric polypeptides described herein). In some embodiments, the subject is identified as being in need of modulation of expression of one or more genes listed in Tables 1-4 in their liver.

In some embodiments, the multi-chain chimeric polypeptide is administered via intramuscular administration, subcutaneous administration, intravenous administration, intrahepatic administration, or intraperitoneal administration.

In some embodiments, the administering results in a decrease (e.g., about a 1% decrease to about a 100% decrease, or any of the subranges of this range described herein) in the expression of one or more (e.g., two or more, three or more, four or more, five or more, six or more, seven or more, eight or more, nine or more, ten or more, fifteen or more, or twenty or more) genes in the liver of the subject selected from the group of: ACSS1, RETN, SLC2A4, PDK4, PNPLA3, GADD45B, PPARGC1A, CAV1, ENDOD1, REG3G, IGHG3, IGHG2B, SCGB3A1, GLYCAM1, IGHG2C, IGKC, LTF, MS4A1, JCHAIN, CD19, IGHM, IFI27L2A, ACKR3, LSP1, PMEPA1, CORO1A, GPX3, MYH8, NPPA, TCAP, FLNC, SLC36A2, MYH6, ACTC1, ACTA2, and TPM2, as compared to the level of expression of the one or more genes in the subject prior to the administering.

In some embodiments, the administering results in an increase (e.g., about a 1% increase to about a 500% increase, about a 1% increase to about a 400% increase, about a 1% increase to about a 300% increase, about a 1% increase to about a 200% increase, about a 1% increase to about a 150% increase, about a 1% increase to about a 100% increase, about a 1% increase to about a 80% increase, about a 1% increase to about a 60% increase, about a 1% increase to about a 40% increase, about a 1% increase to about a 20% increase, about a 1% increase to about a 10% increase, about a 1% increase to about a 5% increase, about a 5% increase to about a 500% increase, about a 5% increase to about a 400% increase, about a 5% increase to about a 300% increase, about a 5% increase to about a 200% increase, about a 5% increase to about a 150% increase, about a 5% increase to about a 100% increase, about a 5% increase to about a 80% increase, about a 5% increase to about a 60% increase, about a 5% increase to about a 40% increase, about a 5% increase to about a 20% increase, about a 5% increase to about a 10% increase, about a 10% increase to about a 500% increase, about a 10% increase to about a 400% increase, about a 10% increase to about a 300% increase, about a 10% increase to about a 200% increase, about a 10% increase to about a 150% increase, about a 10% increase to about a 100% increase, about a 10% increase to about a 80% increase, about a 10% increase to about a 60% increase, about a 10% increase to about a 40% increase, about a 10% increase to about a 20% increase, about a 20% increase to about a 500% increase, about a 20% increase to about a 400% increase, about a 20% increase to about a 300% increase, about a 20% increase to about a 200% increase, about a 20% increase to about a 150% increase, about a 20% increase to about a 100% increase, about a 20% increase to about a 80% increase, about a 20% increase to about a 60% increase, about a 20% increase to about a 40% increase, about a 40% increase to about a 500% increase, about a 40% increase to about a 400% increase, about a 40% increase to about a 300% increase, about a 40% increase to about a 200% increase, about a 40% increase to about a 150% increase, about a 40% increase to about a 100% increase, about a 40% increase to about a 80% increase, about a 40% increase to about a 60% increase, about a 60% increase to about a 500% increase, about a 60% increase to about a 400% increase, about a 60% increase to about a 300% increase, about a 60% increase to about a 200% increase, about a 60% increase to about a 150% increase, about a 60% increase to about a 100% increase, about a 60% increase to about a 80% increase, about a 80% increase to about a 500% increase, about a 80% increase to about a 400% increase, about a 80% increase to about a 300% increase, about a 80% increase to about a 200% increase, about a 80% increase to about a 150% increase, about a 80% increase to about a 100% increase, about a 100% increase to about a 500% increase, about a 100% increase to about a 400% increase, about a 100% increase to about a 300% increase, about a 100% increase to about a 200% increase, about a 100% increase to about a 150% increase, about a 150% increase to about a 500% increase, about a 150% increase to about a 400% increase, about a 150% increase to about a 300% increase, about a 150% increase to about a 200% increase, about a 200% increase to about a 500% increase, about a 200% increase to about a 400% increase, about a 200% increase to about a 300% increase, about a 300% increase to about a 500% increase, about a 300% increase to about a 400% increase, or about a 400% increase to about a 500% increase, in the expression of one or more genes in the liver of the subject selected from the group consisting of: SLC34A2, and CISH, as compared to the level of expression of the one or more genes in the subject prior to the administering.

In some embodiments, the administering results in a decrease (e.g., about a 1% decrease to about a 100% decrease, or any of the subranges of this range described herein) in the expression of one or more (e.g., two or more, three or more, four or more, five or more, six or more, seven or more, eight or more, nine or more, ten or more, fifteen or more, twenty or more, or thirty or more) genes in the liver of the subject selected from the group consisting of: CSF3R, IFI27L2A, GM17066, GNL3, FABP1, GM14303, AURKA, RPL14-PS1, QTRT2, G6PC, C8B, DYNLL1, LCN2, LRG1, CEBPD, COL4A3, ST3GAL5, RSAD2, 9330162G02RIK, PINX1, SRA1, SPATA2L, PNRC1, MUP20, IL6RA, APOA1, IL1B, WDR54, CTCFLOS, GM16973, 4632427E13RIK, IGHG2B, TGFB1I1, SELENBP2, SEMA6B, NEXN, ZFP653, NOB1, PCK1, FAM25C, MAPK15, GM16551, ESM1, RPL37RT, FAM133B, PDE8B, TUT1, S100A11, PDILT, PPARD, IER2, GM15401, MX2, WNK4, G0S2, BC005561, AA986860, JDP2, GM26982, NOP58, ACTB, GM14586, RPP38, GM13436, NT5DC2, EVIPDHL CYTIP, AI846148, CHKA, GM37963, NROB2, CYP4A32, ALKBH2, FAU-PS2, PPP1R15A, KLF2, SLC25A22, GM13341, IGHM, SATB1, SNRPF, DNASE1L2, CD3EAP, GM2788, DANCR, ZFP612, NOP56, JUND, ID1, HSPB1, KLHDC8A, KLF10, ANGPT2, THBS1, GM44891, GM9752, ABLIM3, PTGES, GM28438, 2410002F23RIK, FOSL2, CRIP3, JUN, ALAS1, GM2000, RHOC, LMCD1, GM2061, GM42595, GM11478, IKZF2, PNLDC1, COMTD1, SNORA31, COL20A1, AKAP12, C1QTNF12, 1810032008RIK, 2310033P09RIK, GM47528, SERPINE2, NPFF, SERPINA3K, RFXANK, IGKV5-39, NAB2, MAFF, CEP85, CSAD, LTB4R1, 1810012K08RIK, BCL7C, NRBP2, NLE1, ALKBH1, ARID5A, CFAP43, GM45767, CD8A, PPRC1, GM26870, TMC7, BCL6B, GM16348, GM26981, SLC16A3, TNFRSF12A, CYP2J9, NR4A2, MMP9, MIR17HG, TMEM191C, PCDH11X, HILPDA, RAPGEF4, GM17300, SLC25A47, KCNJ2, NYAP1, LAX1, RPS19-PS3, HES1, RGS16, DUSP1, GM43323, ASB4, MUC6, GM15502, UNG, FOXQ1, GM17936, UBE2C, SLC16A6, MIR7052, NLRP12, GM14286, FGF21, KLFS, GM37969, PF4, GM21738, HOTAIRM1, GM6493, LOR, MFSD2B, MATK, SYNE4, GM44694, TRBC1, GM37274, PLN, CXCR4, PHF24, SNORD104, SERPINA7, RGS4, TCIM, EGFR, GM37760, FBXL22, TEDC2, ENHO, GM26917, GM43775, 4833411C07RIK, GM45053, INHBB, OPN3, SNHG15, B230206H07RIK, KCNE3, GM43305, C530043K16RIK, KLF4, LEPR, JCHAIN, TSKU, LGALS4, PCP4L1, GM44829, DUSP8, GM44620, IGFBP1, JUNB, GM32017, GM2814, GM37144, MYADML2OS, GM37666, HDC, SLFN4, A530041M06RIK, GM43359, GM2602, GM10277, FAM222A, FOXA3, AOC2, SERPINA1E, CTXN1, RAPGEF4OS2, SOCS2, PPAN, PRKAG2OS1, GADD45B, HOXAS, GRHL1, EIF4EBP3, OSGIN1, GM28513, MAP3K6, SLC34A2, B630019A1ORIK, IGKC, PLIN4, ANGPTL4, DUSPS, EGR1, GM42507, GM14257, APOLD1, IER3, ZBTB16, GM37033, IGLC1, GADD45G, IGLC3, GM45244, RGS1, CXCL1, RNF225, GM44005, ANKRD37, NR4A1, GM8893, GM26762, CDKN1A, 5330406M23RIK, IGLV1, IGKV3-2, FOS, GM43637, IGKV3-10, S100A9, GM15622, S100A8, MT1, RETNLG, MT2, IGKV19-93, GM45774, and SERPINA4-PS1, as compared to the level of expression of the one or more genes in the subject prior to the administering.

In some embodiments, the administering results in an increase in the expression of one or more genes in the liver of the subject selected from the group consisting of: DBP, IGKV4-55, PER3, MUP-PS10, GPAM, TMPRSS4, MUP-PS14, AC166078.1, MUP-PS12, GM2065, A530020G20RIK, ACSS2OS, DCLK3, KLF12, GM44669, MFSD9, B4GALNT3, GM3776, TMEM167-PS1, KRT23, LMBRD2, GM22935, SULT2A-PS1, SNAI3, GM15908, MIR6392, ACSS2, NR1D1, BC049987, CCDC85C, CES2C, ACPP, MUP2, PTK6, UGT1A5, 1810008I18RIK, IL22RA1, ACSS3, ADNP, RDH16, SNTB1, 4933411K16RIK, NTRK2, EXTL1, PSTPIP2, RASSF6, AQP4, UGT1A9, PROM1, ZFP608, FAM13A, NFE2, TEF, TNFAIP8L3, SCD1, MMD2, SYNE3, ACLY, C330021F23RIK, STON2, LRFN4, HHIPL1, WNT9B, NR1D2, 1810049J17RIK, PDPR, NA, GM45884, SLC2A5, FAM83F, ZFP526, SGK2, GM43080, DEAF1, MEI, BMF, WDFY2, ADCY9, CLSTN3, ACOT11, LYST, LRTM1, OAT, VPS13C, E330011021RIK, P2RY4, GM11437, RWDD2A, SVIL,

ECHDC1, TRIM14, SLC10A5, TRHDE, MASP1, 2900097C17RIK, NDST1, RDH9, 1110002L01RIK, ABTB2, RGR, ACACB, SACM1L, DYRK2, ROBO1, GM44744, EIF4EBP2, KLHL24, CYP2A5, TIAM2, RAB43, GM13855, 9130409I23RIK, STON1, USP9X, UGT3A1, 9030616G12RIK, DOCKS, KLB, ACE, VLDLR, PCDHGC3, ABCA6, 4932422M17RIK, GM45838, FARP2, GM47205, SP4, UGT1A6B, KLHL28, D130043K22RIK, ASICS, PM20D2, A1CF, SORBS1, SLC10A2, GM10642, UTP14B, GM38394, AFP, INSIG1, HNF1AOS2, METTL4, LSS, MTMR9, HMGCR, GDAP10, ADRA1A, ZFP773, CRKL, CHRNE, STARD13, CRY2, FADS2, COGS, FV1, RCAN2, ABCB1A, PPARA, ATP7A, MVD, 2610037D02RIK, TNFRSF14, SUCNR1, ECI3, ABCC4, LNCBATE1, MINDY2, BTBD7, 4933404012RIK, ABCD1, FMN1, FNIP2, ABHD15, NKX2-6, C77080, GM43611, SGTB, ACSL3, NR5A2, FAM198A, KCTD7, ACACA, ZFP955B, SULT2A3, FZD4, FASN, CYP3A59, ZFP354B, TNFSF10, SESN3, MN1, RNF152, DHCR24, SPHK2, SYTLS, GM6652, BAHCC1, GAREM1, MFSD4A, HGF, GM3571, NOS1AP, DIXDC1, KANK1, REPS2, ASAH2, SEMA3B, RNF103, ZC3H12C, CDS2, DCUN1D4, 2900026A02RIK, CYYR1, EEPD1, P2RY2, CYP2C39, SEC22C, EHHADH, ABCA3, HIPK2, RBM20, GRAIVID4, FCHSD2, MOB3A, HMGN3, KLHDC7A, VCP-RS, TERT, CYP3A41B, ARL13B, ZC3H12D, TLCD2, SNHG11, SORL1, GPR157, DNAJA4, TMEM253, TACO1, SPATA5L1, RHBG, COL15A1, PCDH12, IRS1, ASCC3, KIF16B, and MR1, as compared to the level of expression of the one or more genes in the subject prior to the administering.

In some embodiments, the subject has been previously identified or diagnosed as having a liver disease (e.g., any of the exemplary liver diseases described herein or known in the art) or a metabolic syndrome (e.g., any of the exemplary metabolic syndromes described herein or known in the art). In some embodiments, the subject has been previously identified or diagnosed as having a liver disease (e.g., any of the exemplary liver diseases described herein or known in the art). In some embodiments, the liver disease is selected from the group of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease. In some embodiments, the subject has been previously identified or diagnosed as having a metabolic syndrome (e.g., any of the exemplary metabolic syndromes described herein or known in the art). In some embodiments, the metabolic syndrome is selected from the group of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

In some embodiments, the subject has not been previously identified or diagnosed as having type 2 diabetes mellitus. In some embodiments, the subject has not been previously identified or diagnosed as having adipose atrophy. In some embodiments, the subject has not been previously identified or diagnosed as having lipodystrophy. In some embodiments, the subject has not been previously identified or diagnosed as having liver cirrhosis. In some embodiments, the subject has not been previously identified or diagnosed as having NAFLD. In some embodiments, the subject has not been previously identified or diagnosed as having non-alcoholic steatohepatitis.

### Additional Therapeutic Agents

Some embodiments of any of the methods described herein can further include administering to a subject (e.g., any of the subjects described herein) a therapeutically effective amount of one or more additional therapeutic agents. The one or more additional therapeutic agents can be administered to the subject at substantially the same time as the multi-chain chimeric polypeptide (e.g., any of the multi-chain chimeric polypeptides described herein). In some embodiments, one or more additional therapeutic agents can be administered to the subject prior to administration of the multi-chain chimeric polypeptide (e.g., any of the multi-chain chimeric polypeptides described herein). In some embodiments, one or more additional therapeutic agents can be administered to the subject after administration of the multi-chain chimeric polypeptide (e.g., any of the multi-chain chimeric polypeptides described herein) to the subject.

Non-limiting examples of additional therapeutic agents include: anti-inflammatory agents, anti-cancer drugs, activating receptor agonists, immune checkpoint inhibitors, agents for blocking HLA-specific inhibitory receptors, Glucogen Synthase Kinase (GSK) 3 inhibitors, antibodies, and ex-vivo activated immune cells.

Non-limiting examples of anticancer drugs include antimetabolic drugs (e.g., 5-fluorouracil (5-FU), 6-mercaptopurine (6-MP), capecitabine, cytarabine, floxuridine, fludarabine, gemcitabine, hydroxycarbamide, methotrexate, 6-thioguanine, cladribine, nelarabine, pentostatin, or pemetrexed), plant alkaloids (e.g., vinblastine, vincristine, vindesine, camptothecin, 9-methoxycamptothecin, coronaridine, taxol, naucleaorals, diprenylated indole alkaloid, montamine, schischkiniin, protoberberine, berberine, sanguinarine, chelerythrine, chelidonine, liriodenine, clivorine, (3-carboline, antofine, tylophorine, cryptolepine, neocryptolepine, corynoline, sampangine, carbazole, crinamine, montanine, ellipticine, paclitaxel, docetaxel, etoposide, tenisopide, irinotecan, topotecan, or acridone alkaloids), proteasome inhibitors (e.g., lactacystin, disulfiram, epigallocatechin-3-gallate, marizomib (salinosporamide A), oprozomib (ONX-0912), delanzomib (CEP-18770), epoxomicin, MG132, beta-hydroxy beta-methylbutyrate, bortezomib, carfilzomib, or ixazomib), antitumor antibiotics (e.g., doxorubicin, daunorubicin, epirubicin, mitoxantrone, idarubicin, actinomycin, plicamycin, mitomycin, or bleomycin), histone deacetylase inhibitors (e.g., vorinostat, panobinostat, belinostat, givinostat, abexinostat, depsipeptide, entinostat, phenyl butyrate, valproic acid, trichostatin A, dacinostat, mocetinostat, pracinostat, nicotinamide, cambinol, tenovin 1, tenovin 6, sirtinol, ricolinostat, tefinostat, kevetrin, quisinostat, resminostat, tacedinaline, chidamide, or selisistat), tyrosine kinase inhibitors (e.g., axitinib, dasatinib, encorafinib, erlotinib, imatinib, nilotinib, pazopanib, and sunitinib), and chemotherapeutic agents (e.g., all-trans retinoic acid, azacitidine, azathioprine, doxifluridine, epothilone, hydroxyurea, imatinib, teniposide, tioguanine, valrubicin, vemurafenib, and lenalidomide). Additional examples of chemotherapeutic agents include alkylating agents, e.g., mechlorethamine, cyclophosphamide, chlorambucil, melphalan, ifosfamide, thiotepa, hexamethylmelamine, busulfan, altretamine, procarbazine, dacarbazine, temozolomide, carmustine, lumustine, streptozocin, carboplatin, cisplatin, and oxaliplatin.

Non-limiting examples of activating receptor agonists include any agonists for activating receptors which activate and enhance the cytotoxicity of NK cells, including anti-CD16 antibodies (e.g., anti-CD16/CD30 bispecific monoclonal antibody (BiMAb)) and Fc-based fusion proteins. Non-limiting examples of checkpoint inhibitors include anti-PD-1 antibodies (e.g., MEDI0680), anti-PD-Ll antibodies (e.g., BCD-135, BGB-A333, CBT-502, CK-301, CS1001, FAZ053, KN035, MDX-1105, MSB2311, SHR-1316, anti-PD-L1/CTLA-4 bispecific antibody KN046, anti-PD-Ll/TGF(3RII fusion protein M7824, anti-PD-L1/TIM-3 bispecific antibody LY3415244, atezolizumab, or avelumab), anti-TIM3 antibodies (e.g., TSR-022, Sym023, or MBG453) and anti-CTLA-4 antibodies (e.g., AGEN1884, MK-1308, or an anti-CTLA-4/OX40 bispecific antibody ATOR-1015). Non-limiting examples of agents for blocking HLA-specific inhibitory receptors include monalizumab (e.g., an anti-HLA-E NKG2A inhibitory receptor monoclonal antibody). Non-limiting examples of GSK3 inhibitor include tideglusib or CHIR99021. Non-limiting examples of antibodies that can be used as additional therapeutic agents include anti-CD26 antibodies (e.g., YS110), anti-CD36 antibodies, and any other antibody or antibody construct that can bind to and activate an Fc receptor (e.g., CD16) on a NK cell. In some embodiments, an additional therapeutic agent can be insulin or metformin.

Non-limiting examples of in-vitro activated immune cells include regulatory T cells, CAR-regulatory T cells, NK cells, CAR-NK cells, cytotoxic T cells, and CAR-cytotoxic T cells.

### EXAMPLES

The invention is further described in the following examples, which do not limit the scope of the invention described in the claims.

### Example 1

### Construction of Exemplary Multi-Chain Chimeric Polypeptides and Evaluation of Properties Thereof

Two multi-chain chimeric polypeptides were generated and their properties were evaluated. Each of the two multi-chain chimeric polypeptides includes a first chimeric polypeptide that includes a soluble tissue factor domain covalently linked a first target-binding domain and a first domain of an affinity pair of domains. The second chimeric polypeptide in each of the two multi-chain chimeric polypeptides includes a second domain of the affinity pair of domains, and a second target-binding domain.

### Description of Logic Underlying Construction of Multi-Chain Chimeric Polypeptides

Tissue Factor (TF) is a stable, transmembrane protein containing 236 amino acid residues. The truncated, recombinant 219-amino-acid extracellular domain of tissue factor is soluble and is known to be expressed at high levels in bacteria or mammalian cells. Without wishing to be bound to a particular theory, the applicants speculated that the 219-aa tissue factor could be used as a connector linker for creation of unique multi-chain chimeric polypeptides.

First chimeric polypeptides including soluble tissue factor domain were produced at high levels by CHO cells grown in fermentation broth. These first chimeric polypeptides were purified by an anti-tissue factor monoclonal antibody (mAb) coupled on a solid matrix. Notably, tissue factor contains binding sites for FVIIa and FX. The catalytic activity of the tissue factor-FVIIa complex for FX is approximately 1 million-fold lower when tissue factor is not anchored to a phospholipid bilayer. Thus, without wishing to be bound to a particular theory, applicants speculated that using the 219-aa extracellular domain of tissue factor without the transmembrane in construction of the first chimeric polypeptides may eliminate the pro-coagulation activity of tissue factor in the first chimeric polypeptides. In an effort to further reduce or eliminate the pro-coagulation activity of the 219-aa tissue factor, select mutations in tissue factor can be made, specifically at seven amino acid residues that are known to contribute to binding energy of the FVIIa binding site.

### Characterization of Binding Interactions for Described Chimeric Polypeptides

To determine if the first and second chimeric polypeptides bind to each other to form multi-chain chimeric polypeptides, in vitro binding assays were performed. To determine if the first chimeric polypeptide comprising soluble tissue factor domain are recognized and bound by anti-TF mAb, in vitro binding assays were performed. Notably, the data indicated that the mutated tissue factor proteins are still recognized and selectively bound by the anti-TF mAb which is known to bind to the FX binding site on tissue factor. To determine if the first chimeric polypeptides comprising soluble tissue factor domain covalently linked to scFvs or cytokines (see FIG. 1 and FIG. 2) possess functional scFvs or cytokines, in vitro binding assays were performed. The data from the aforementioned assays were consistent with the purified first chimeric polypeptides having the expected biological activities (e.g. scFvs selectively bind expected target antigens or cytokines selectively bind expected receptors or binding proteins).

In addition, experiments performed using the two multi-chain chimeric polypeptides including a first and second chimeric polypeptide bound to each other demonstrate the expected target binding activity (e.g., the multi-chain chimeric polypeptide binds specifically to the target specifically recognized by the first target-binding domain and the target specifically recognized by the second target-binding domain).

Based on the aforementioned results, applicants concluded that the soluble tissue factor connecter linker provided or enabled appropriate display of the polypeptides encoding either scFvs, interleukins, cytokines, interleukin receptors, or cytokine receptors in three-dimensional space relative to soluble tissue factor domain and relative to one another such that each retained expected biological properties and activities.

When both the first and second chimeric polypeptides were co-expressed, the heterodimeric complexes were secreted into the fermentation broths at high levels. The complexes were captured and readily purified by anti-TF mAb conjugated to a solid matrix using affinity chromatography. The first and second target-binding domains of these multi-chain chimeric polypeptides retained their expected biological activities as assayed by in vitro binding assays. Thus, the assembly of the multi-chain chimeric polypeptides provides the appropriate spatial display and folding of the domains for biological activities. Importantly, the spatial arrangement of the multi-chain chimeric polypeptides does not interfere with the FX binding site on tissue factor which enables the use of anti-TF mAb for affinity purification.

### Characterization of Stability for Described Chimeric Polypeptides

Both purified multi-chain chimeric polypeptides are stable. These multi-chain chimeric polypeptides are structurally intact and fully biologically active when they are incubated in human serum at 37° C. for 72 hours.

### Characterization of Propensity of Described Chimeric Polypeptides to Aggregate

Both purified multi-chain chimeric polypeptides developed do not form aggregates when stored at 4° C. in PBS.

### Characterization of Viscosity of Described Chimeric Polypeptides

There is no viscosity issue when the multi-chain chimeric polypeptides are formulated at a concentration as high as 50 mg/mL in PBS.

### Additional Applications of the Multi-Chain Chimeric Polypeptide Platform

The data from these studies show that the platform technologies described herein can be utilized to create molecules that could be fused to target-binding domains derived from antibodies, in any of the formats as described herein including, without limitation, adhesion molecules, receptors, cytokines, ligands, and chemokines. With the appropriate target-binding domain, the resulting multi-chain chimeric polypeptides could promote conjugation of various immune effector cells and mediate destruction of target cells, including cancer cells, virally-infected cells, or senescent cells. Other domains in the multi-chain chimeric polypeptides stimulate, activate, and attract the immune system for enhancing cytotoxicity of effector cells for the targeted cells.

### Example 2

### TGFRt15-TGFRs Fusion Protein Generation and Characterization

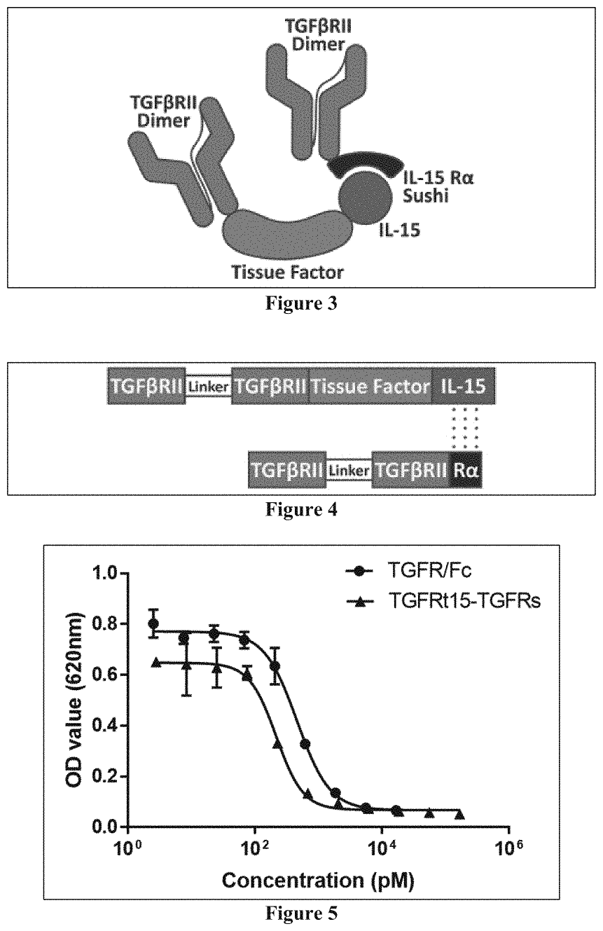

A fusion protein complex was generated comprising of TGFβ Receptor II/IL-15RαSu and TGFβ Receptor II/TF/IL-15 fusion proteins (FIG. 3 and FIG. 4). The human TGFP Receptor II (Ile24-Asp159), tissue factor 219, and IL-15 sequences were obtained from the UniProt website and DNA for these sequences was synthesized by Genewiz. Specifically, a construct was made linking two TGFβ Receptor II sequences with a G45(3) linker to generate a single chain version of TGFβ Receptor II and then directly linking to the N-terminus coding region of tissue factor 219 followed by the N-terminus coding region of IL-15.

The nucleic acid and protein sequences of a construct comprising two TGFβ Receptor II linked to the N-terminus of tissue factor 219 following with the N-terminus of IL-15 are shown below.

The nucleic acid sequence of the two TGFβ Receptor II/TF/IL-15 construct (including signal peptide sequence) is as follows:

(Signal peptide)
ATGAAGTGGGTGACCTTCATCAGCCTGCTGTTCCT
GTTCTCCAGCGCCTACTCC
(Two Human TGFβ Receptor II fragments)
ATCCCCCCCCATGTGCAAAAGAGCGTGAACAACGA
TATGATCGTGACCGACAACAACGGCGCCGTGAAGT
TTCCCCAGCTCTGCAAGTTCTGCGATGTCAGGTTC
AGCACCTGCGATAATCAGAAGTCCTGCATGTCCAA
CTGCAGCATCACCTCCATCTGCGAGAAGCCCCAAG
AAGTGTGCGTGGCCGTGTGGCGGAAAAATGACGAG
AACATCACCCTGGAGACCGTGTGTCACGACCCCAA
GCTCCCTTATCACGACTTCATTCTGGAGGACGCTG
CCTCCCCCAAATGCATCATGAAGGAGAAGAAGAAG
CCCGGAGAGACCTTCTTTATGTGTTCCTGTAGCAG
CGACGAGTGTAACGACAACATCATCTTCAGCGAAG
AGTACAACACCAGCAACCCTGATGGAGGTGGCGGA
TCCGGAGGTGGAGGTTCTGGTGGAGGTGGGAGTAT
TCCTCCCCACGTGCAGAAGAGCGTGAATAATGACA
TGATCGTGACCGATAACAATGGCGCCGTGAAATTT
CCCCAGCTGTGCAAATTCTGCGATGTGAGGTTTTC
CACCTGCGACAACCAGAAGTCCTGTATGAGCAACT
GCTCCATCACCTCCATCTGTGAGAAGCCTCAGGAG
GTGTGCGTGGCTGTCTGGCGGAAGAATGACGAGAA
TATCACCCTGGAAACCGTCTGCCACGATCCCAAGC
TGCCCTACCACGATTTCATCCTGGAAGACGCCGCC
AGCCCTAAGTGCATCATGAAAGAGAAAAAGAAGCC
TGGCGAGACCTTTTTCATGTGCTCCTGCAGCAGCG
ACGAATGCAACGACAATATCATCTTTAGCGAGGAA
TACAATACCAGCAACCCCGAC
(Human Tissue Factor 219)
AGCGGCACAACCAACACAGTCGCTGCCTATAACCT
CACTTGGAAGAGCACCAACTTCAAAACCATCCTCG
AATGGGAACCCAAACCCGTTAACCAAGTTTACACC
GTGCAGATCAGCACCAAGTCCGGCGACTGGAAGTC
CAAATGTTTCTATACCACCGACACCGAGTGCGATC
TCACCGATGAGATCGTGAAAGATGTGAAACAGACC
TACCTCGCCCGGGTGTTTAGCTACCCCGCCGGCAA
TGTGGAGAGCACTGGTTCCGCTGGCGAGCCTTTAT
ACGAGAACAGCCCCGAATTTACCCCTTACCTCGAG
ACCAATTTAGGACAGCCCACCATCCAAAGCTTTGA
GCAAGTTGGCACAAAGGTGAATGTGACAGTGGAGG
ACGAGCGGACTTTAGTGCGGCGGAACAACACCTTT
CTCAGCCTCCGGGATGTGTTCGGCAAAGATTTAAT
CTACACACTGTATTACTGGAAGTCCTCTTCCTCCG
GCAAGAAGACAGCTAAAACCAACACAAACGAGTTT
TTAATCGACGTGGATAAAGGCGAAAACTACTGTTT
CAGCGTGCAAGCTGTGATCCCCTCCCGGACCGTGA
ATAGGAAAAGCACCGATAGCCCCGTTGAGTGCATG
GGCCAAGAAAAGGGCGAGTTCCGGGAG
(Human IL-15)
(SEQ ID NO: 59)
AACTGGGTGAACGTCATCAGCGATTTAAAGAAGAT
CGAAGATTTAATTCAGTCCATGCATATCGACGCCA
CTTTATACACAGAATCCGACGTGCACCCCTCTTGT
AAGGTGACCGCCATGAAATGTTTTTTACTGGAGCT
GCAAGTTATCTCTTTAGAGAGCGGAGACGCTAGCA
TCCACGACACCGTGGAGAATTTAATCATTTTAGCC
AATAACTCTTTATCCAGCAACGGCAACGTGACAGA
GTCCGGCTGCAAGGAGTGCGAAGAGCTGGAGGAGA
AGAACATCAAGGAGTTTCTGCAATCCTTTGTGCAC
ATTGTCCAGATGTTCATCAATACCTCC

The amino acid sequence of TGFβ Receptor II/TF/IL-15 fusion protein (including the leader sequence) is as follows:

(Signal peptide)
MKWVTFISLLFLF S SAYS
(Human TGFβ Receptor II)
IPPHVQKSVNNDMIVTDNNGAVKFPQLCKFCDVRF
STCDNQKSCMSNCSITSICEKPQEVCVAVWRKNDE
NITLETVCHDPKLPYHDFILEDAASPKCIMKEKKK
PGETFFMCSCSSDECNDNIIFSEEYNTSNPDGGGG
SGGGGSGGGGSIPPHVQKSVNNDMIVTDNNGAVKF
PQLCKFCDVRFSTCDNQKSCMSNCSITSICEKPQE
VCVAVWRKNDENITLETVCHDPKLPYHDFILEDAA
SPKCIMKEKKKPGETFFMCSCSSDECNDNIIFSEE
YNTSNPD
(Human Tissue Factor 219)
SGTTNTVAAYNLTWKSTNFKTILEWEPKPVNQVYT
VQISTKSGDWKSKCFYTTDTECDLTDEIVKDVKQT
YLARVFSYPAGNVESTGSAGEPLYENSPEFTPYLE
TNLGQPTIQSFEQVGTKVNVTVEDERTLVRRNNTF
LSLRDVFGKDLIYTLYYWKSSSSGKKTAKTNTNEF
LIDVDKGENYCFSVQAVIPSRTVNRKSTDSPVECM
GQEKGEFRE
(Human IL-15)
(SEQ ID NO: 7)
NWVNVISDLKKIEDLIQSMHIDATLYTESDVHPSC
KVTAMKCFLLELQVISLESGDASIHDTVENLIILA
NNSLSSNGNVTESGCKECEELEEKNIKEFLQSFVH
IVQMFINTS

Constructs were also made by attaching two TGFβ Receptor II directly to the IL-20 15RαSu chain which was synthesized by Genewiz. The nucleic acid and protein sequences of a construct comprising the TGFβ Receptor II linked to the N-terminus of IL-15RαSu are shown below.

The nucleic acid sequence of the TGFβ Receptor II/IL-15 RαSu construct (including signal peptide sequence) is as follows:

(Signal peptide)
ATGAAGTGGGTGACCTTCATCAGCCTGCTGTTCCT
GTTCTCCAGCGCCTACTCC
(Two human TGFβ Receptor II fragments)
ATCCCCCCCCATGTGCAAAAGAGCGTGAACAACGA
TATGATCGTGACCGACAACAACGGCGCCGTGAAGT
TTCCCCAGCTCTGCAAGTTCTGCGATGTCAGGTTC
AGCACCTGCGATAATCAGAAGTCCTGCATGTCCAA
CTGCAGCATCACCTCCATCTGCGAGAAGCCCCAAG
AAGTGTGCGTGGCCGTGTGGCGGAAAAATGACGAG
AACATCACCCTGGAGACCGTGTGTCACGACCCCAA
GCTCCCTTATCACGACTTCATTCTGGAGGACGCTG
CCTCCCCCAAATGCATCATGAAGGAGAAGAAGAAG
CCCGGAGAGACCTTCTTTATGTGTTCCTGTAGCAG
CGACGAGTGTAACGACAACATCATCTTCAGCGAAG
AGTACAACACCAGCAACCCTGATGGAGGTGGCGGA
TCCGGAGGTGGAGGTTCTGGTGGAGGTGGGAGTAT
TCCTCCCCACGTGCAGAAGAGCGTGAATAATGACA
TGATCGTGACCGATAACAATGGCGCCGTGAAATTT
CCCCAGCTGTGCAAATTCTGCGATGTGAGGTTTTC
CACCTGCGACAACCAGAAGTCCTGTATGAGCAACT
GCTCCATCACCTCCATCTGTGAGAAGCCTCAGGAG
GTGTGCGTGGCTGTCTGGCGGAAGAATGACGAGAA
TATCACCCTGGAAACCGTCTGCCACGATCCCAAGC
TGCCCTACCACGATTTCATCCTGGAAGACGCCGCC
AGCCCTAAGTGCATCATGAAAGAGAAAAAGAAGCC
TGGCGAGACCTTTTTCATGTGCTCCTGCAGCAGCG
ACGAATGCAACGACAATATCATCTTTAGCGAGGAA
TACAATACCAGCAACCCCGAC
(Human IL-15R a sushi domain)
(SEQ ID NO: 61)
ATTACATGCCCCCCTCCCATGAGCGTGGAGCACGC
CGACATCTGGGTGAAGAGCTATAGCCTCTACAGCC
GGGAGAGGTATATCTGTAACAGCGGCTTCAAGAGG
AAGGCCGGCACCAGCAGCCTCACCGAGTGCGTGCT
GAATAAGGCTACCAACGTGGCTCACTGGACAACAC
CCTCTTTAAAGTGCATCCGG

The amino acid sequence of the two TGFβ Receptor IFIL-15RαSu construct (including signal peptide sequence) is as follows:

(Signal peptide)
MKWVTFISLLFLFSSAYS
(Two human TGFβ Receptor II
extra-cellular domains)
IPPHVQKSVNNDMIVTDNNGAVKFPQLCKFCDVRF
STCDNQKSCMSNCSITSICEKPQEVCVAVWRKNDE
NITLETVCHDPKLPYHDFILEDAASPKCIMKEKKK
PGETFFMCSCSSDECNDNIIFSEEYNTSNPDGGGG
SGGGGSGGGGSIPPHVQKSVNNDMIVTDNNGAVKF
PQLCKFCDVRFSTCDNQKSCMSNCSITSICEKPQE
VCVAVWRKNDENITLETVCHDPKLPYHDFILEDAA
SPKCIMKEKKKPGETFFMCSCSSDECNDNIIFSEE
YNTSNPD
(Human IL-15R a sushi domain)
(SEQ ID NO: 8)
ITCPPPMSVEHADIWVKSYSLYSRERYICNSGFKR
KAGTSSLTECVLNKATNVAHWTTPSLKCIR

In some cases, the leader peptide is cleaved from the intact polypeptide to generate the mature form that may be soluble or secreted.

The TGFβR/IL-15RαSu and TGFβR/TF/IL-15 constructs were cloned into a modified retrovirus expression vectors as described previously (Hughes M S, Yu Y Y, Dudley M E, Zheng Z, Robbins P F, Li Y, et al. Transfer of a TCR gene derived from a patient with a marked antitumor response conveys highly active T-cell effector functions. Hum Gene Ther 2005;16:457-72), and the expression vectors were transfected into CHO-K1 cells. Co-expression of the two constructs in CHO-K1 cells allowed for formation and secretion of the soluble TGFβR/TF/IL-15:TGFβR/IL-15RαSu protein complex (referred to as TGFRt15-TGFRs), which can be purified by anti-TF IgG1 affinity and other chromatography methods.

Effect of TGFRt15-TGFRs on TGFβ1 activity in HEK-Blue TGFβ cells

To evaluate the activity of TGFβRII in TGFRt15-TGFRs, the effect of TGFRt15-TGFRs on the activity of TGFβ1 in HEK-Blue TGFβ cells was analyzed. HEK-Blue TGFβ cells (Invivogen) were washed twice with pre-warmed PBS and resuspended in the testing medium (DMEM, 10% heat-inactivated FCS, 1× glutamine, 1× anti-anti, and 2× glutamine) at 5×10⁵ cells/mL. In a flat-bottom 96-well plate, 50 μL cells were added to each well (2.5×10⁴ cells/well) and followed with 50 μL 0.1nM TGFβ1 (R&D systems). TGFRt15-TGFRs or TGFR-Fc (R&D Systems) prepared at a 1:3 serial dilution was then added to the plate to reach a total volume of 200 μL. After 24hrs of incubation at 37° C., 40 μL of induced HEK-Blue TGFβ cell supernatant was added to 160 μL pre-warmed QUANTI-Blue (Invivogen) in a flat-bottom 96-well plate, and incubated at 37° C. for 1-3 hrs. The OD values were then determined using a plate reader (Multiscan Sky) at 620-655 nM. The IC₅₀ of each protein sample was calculated with GraphPad Prism 7.04. The IC₅₀ of TGFRt15-TGFRs and TGFR-Fc were 216.9 pM and 460.6 pM respectively. These results showed that the TGFβRII domain in TGFRt15-TGFRs was able to block the activity of TGFβ1 in HEK-Blue TGFβ cells (FIG. 5).

The IL-15 in TGFRt15-TGFRs promotes IL-2Rβ and common γ chain containing 32Dβ cell proliferation

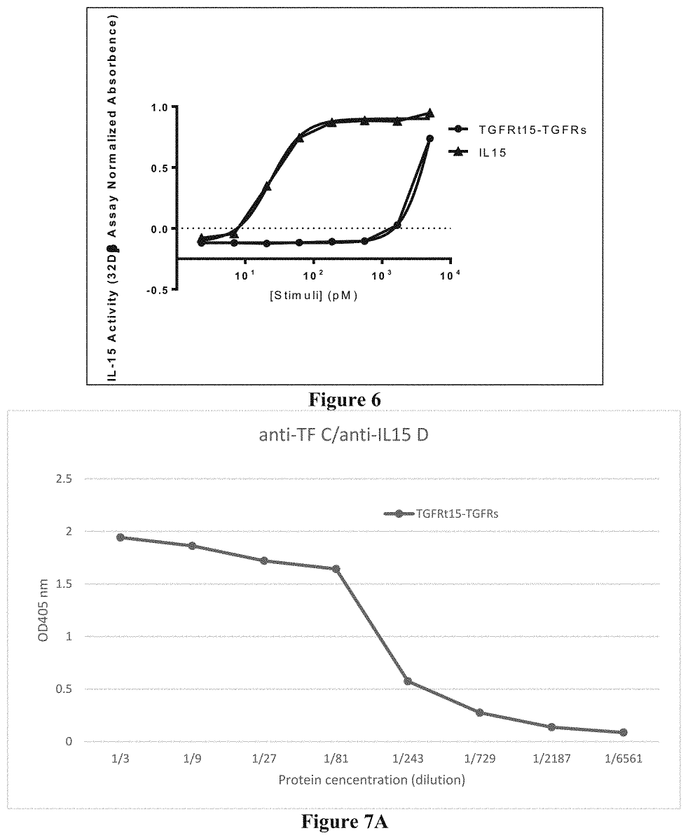

To evaluate the activity of IL-15 in TGFRt15-TGFRs, the IL-15 activity of TGFRt15-TGFRs was compared to recombinant IL-15 using 32Dβ cells that express IL2Rβ and common y chain, and evaluating their effects on promoting cell proliferation. IL-15 dependent 32Dβ cells were washed 5 times with IMDM-10% FBS and seeded in the wells at 2×10⁴ cells/well. Serially-diluted TGFRt15-TGFRs or IL-15 were added to the cells (FIG. 6). Cells were incubated in a CO₂ incubator at 37° C. for 3 days. Cell proliferation was detected by adding 10 μL of WST1 to each well on day 3 and incubating for an additional 3 hours in a CO₂ incubator at 37° C. The absorbance at 450 rim was measured by analyzing the amount of formazan dye produced. As shown in FIG. 5, TGFRt15-TGFRs and promoted 32Dβ cell proliferation, with the EC₅₀ of TGFRt15-TGFRs and IL-15 being 1901 pM and 10.63 pM, respectively.

Detection of IL-15 and TGFβRII domains in TGFRt15-TGFRs with corresponding antibodies using ELISA

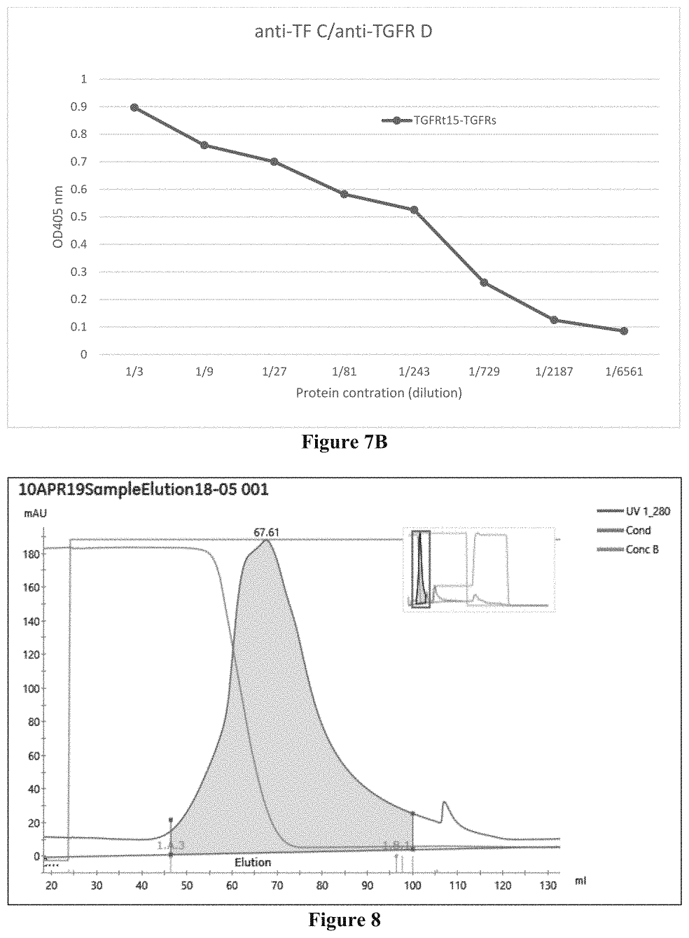

A 96-well plate was coated with 100 μL (8 μg/mL) of anti-TF IgG1 in R5 (coating buffer) and incubated at room temperature (RT) for 2 hrs. The plates were washed 3 times and blocked with 100 μL of 1% BSA in PBS. TGFRt15-TGFRs was added at a 1:3 serial dilution, and incubated at RT for 60 min. After 3 washes, 50 ng/mL of biotinylated-anti-IL-15 antibody (BAM247, R&D Systems), or 200 ng/mL of biotinylated-anti-TGFβRII antibody (BAF241, R&D Systems) was added to the wells and incubated at RT for 60 min. Next the plates were washed 3 times, and 0.25 μg/mL of HRP-SA (Jackson ImmunoResearch) at 100 μL per well was added and incubated for 30 min at RT, followed by 4 washes and incubation with 100 μL of ABTS for 2 mins at RT. Absorbance at 405 nm was read. As shown in FIG. 7A and 7B, the IL-15 and TGFβRII domains in TGFRt15-TGFRs were detected by the individual antibodies.

Purification Elution Chromatograph of TGFRt15-TGFRs from Anti-TF Antibody Affinity Column

TGFRt15-TGFRs harvested from cell culture was loaded onto the anti-TF antibody affinity column equilibrated with 5 column volumes of PBS. After sample loading, the column was washed with 5 column volumes of PBS, followed by elution with 6 column volumes of 0.1M acetic acid (pH 2.9). A280 elution peak was collected and then neutralized to pH 7.5-8.0 with 1M Tris base. The neutralized sample was then buffer exchanged into PBS using Amicon centrifugal filters with a 30 KDa molecular weight cutoff. As shown in FIG. 8, the anti-TF antibody affinity column bound to TGFRt15-TGFRs which contains TF as a fusion partner. The buffer-exchanged protein sample was stored at 2-8° C. for further biochemical analyses and biological activity tests. After each elution, the anti-TF antibody affinity column was stripped using 6 column volumes of 0.1M glycine (pH 2.5). The column was then neutralized using 5 column volumes of PBS, and 7 column volumes of 20% ethanol for storage. The anti-TF antibody affinity column was connected to a GE Healthcare AKTA Avant system. The flow rate was 4 mL/min for all steps except for the elution step, which was 2 mL/min.

### Analytical Size Exclusion Chromatography (SEC) Analysis of TGFRt15-TGFRs

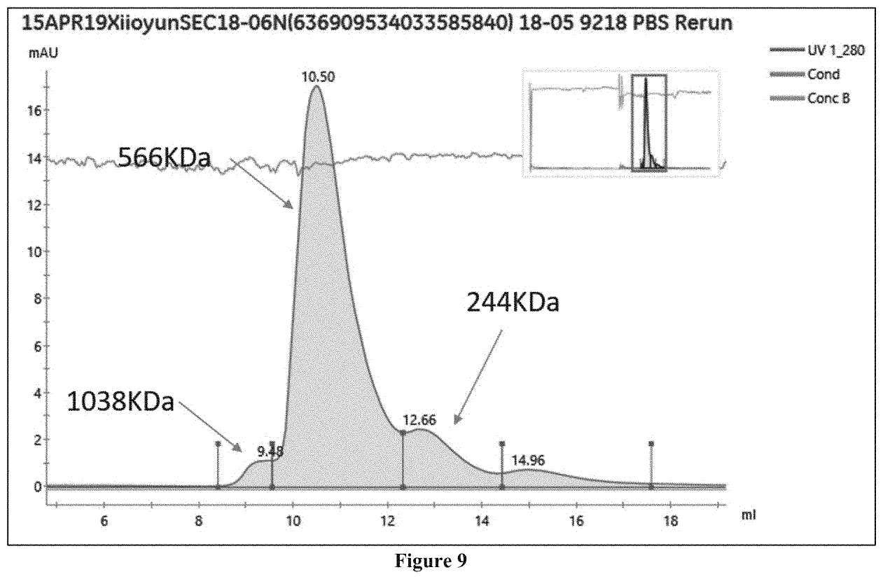

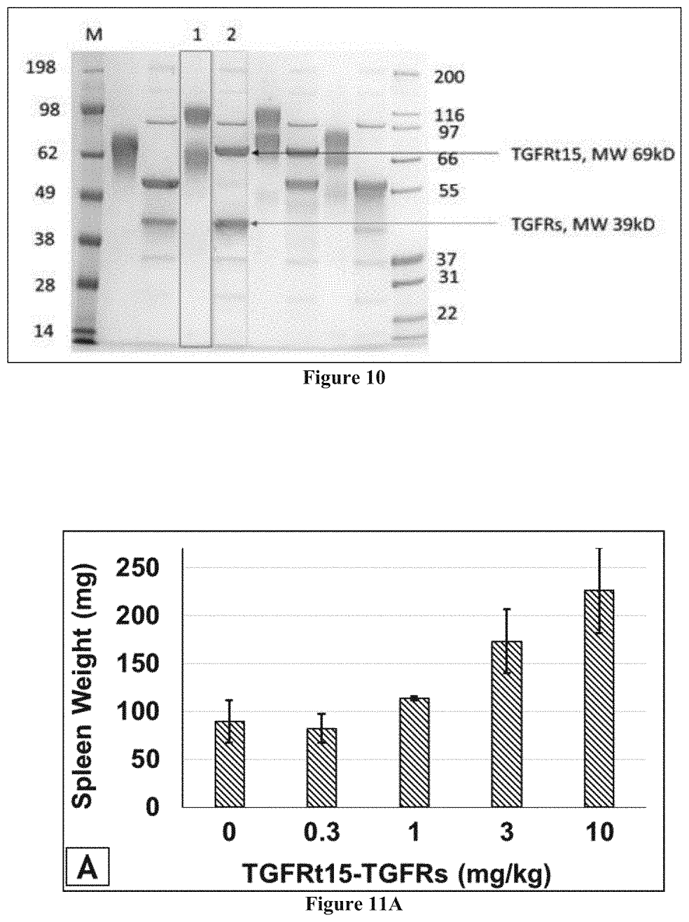

A Superdex 200 Increase 10/300 GL gel filtration column (from GE Healthcare) was connected to an AKTA Avant system (from GE Healthcare). The column was equilibrated with 2 column volumes of PBS. The flow rate was 0.7 mL/min. A sample containing TGFRt15-TGFRs in PBS was injected into the Superdex 200 column using a capillary loop, and analyzed by SEC. The SEC chromatograph of the sample is shown in FIG. 9. The SEC results showed four protein peaks for TGFRt15-TGFRs.

### Reduced SDS PAGE Analysis of TGFRt15-TGFRs

To determine the purity and molecular weight of the TGFRt15-TGFRs protein, protein sample purified with anti-TF antibody affinity column was analyzed by sodium dodecyl sulfate polyacrylamide gel (4-12% NuPage Bis-Tris gel) electrophoresis (SDS-PAGE) method under reduced condition. After electrophoresis, the gel was stained with InstantBlue for about 30 min, followed by destaining overnight in purified water.

To verify that the TGFRt15-TGFRs protein undergoes glycosylation after translation in CHO cells, a deglycosylation experiment was conducted using the Protein Deglycosylation Mix 1:11 kit from New :England Biolabs and the manufacturer's instructions. FIG. 10 shows the reduced SDS-PAGE analysis of the sample in non-deglycosylated (lane 1 in red outline) and deglycosylated (lane 2 in yellow outline) state. The results showed that the TGFRt15-TGFRs protein is glycosylated when expressed in MO cells. Alter deglycosylation, the purified sample showed expected molecular weights (69 kDa and 39 kDa) in the reduced SDS gel. Lane M was loaded with 10 ul of SeeBlue Plus2 Prestained Standard.

Immunostimulatory Activity of TGFRt15-TGFRs in C57BL/6 mice

TGFRt15-TGFRs is a multi-chain polypeptide (a type A multi-chain polypeptide described herein) that includes a first polypeptide that is a soluble fusion of two TGFβRII domains, human tissue factor 219 fragment and human IL-15, and the second polypeptide that is a soluble fusion of two TGFPRII domains and sushi domain of human IL-15 receptor alpha chain.

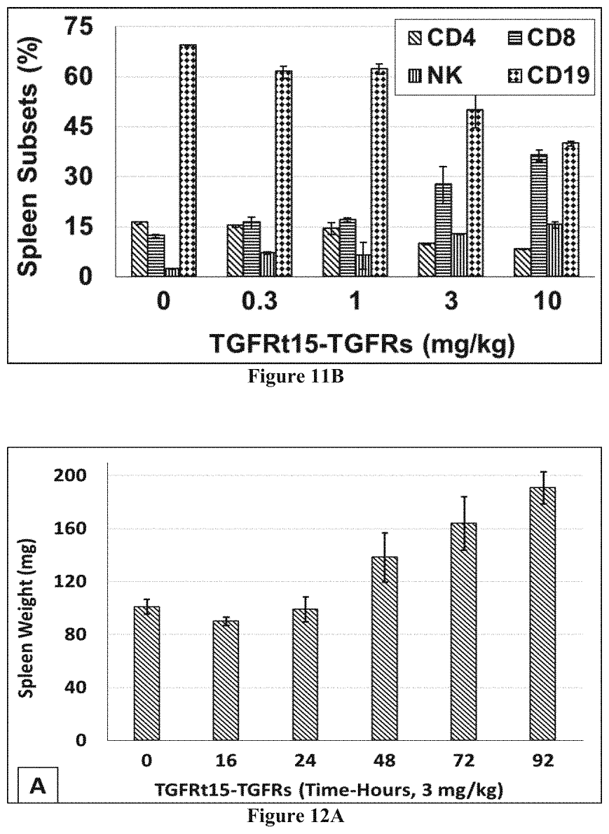

Wild type C57BL/6 mice were treated subcutaneously with either control solution or with TGFRt15-TGFRs at a dosage of 0.3 mg/kg, 1 mg/kg, 3 mg/kg, or 10 mg/kg. Four days after treatment, spleen weight and the percentages of various immune cell types present in the spleen were evaluated. As shown in FIG. 11A, the spleen weight in mice treated with TGFRt15-TGFRs increased with increasing dosage of TGFRt15-TGFRs. Moreover, the spleen weight in mice treated with 1 mg/kg, 3 mg/kg, and 10 mg/kg of TGFRt15-TGFRs were higher as compared to mice treated with the control solution, respectively. In addition, the percentages of CD4⁺ T cells, CDS⁺ T cells, NK cells, and CD19⁺ B cells present in the spleen of control-treated and TGFRt15-TGFRs-treated mice were evaluated. As shown in FIG. 11B, in the spleens of mice treated with TGFRt15-TGFRs, the percentages of CDS⁺ T cells and NK cells both increased with increasing dosage of TGFRt15-TGFRs. Specifically, the percentages of CDS⁺T cells were higher in mice treated with 0.3 mg/kg, 3 mg/kg, and 10 mg/kg of TGFRt15-TGFRs compared to control-treated mice, and the percentages of NK cells were higher in mice treated with 0.3 mg/kg, 1 mg/kg, 3 mg/kg, and 10 mg/kg of TGFRt15-TGFRs compared to control-treated mice. These results demonstrate that TGFRt15-TGFRs is able to stimulate immune cells in the spleen, in particular CD8⁺ T cells and NK cells.

The pharmacokinetics of TGFRt15-TGFRs molecules were evaluated in wild type C57BL/6 mice. The mice were treated subcutaneously with TGFRt15-TGFRs at a dosage of 3 mg/kg. The mouse blood was drained from tail vein at various time points and the serum was prepared. The TGFRt15-TGFRs concentrations in mouse serum was determined with ELISA (capture: anti-human tissue factor antibody; detection: biotinylated anti-human TGFβ receptor antibody and followed by peroxidase conjugated streptavidin and ABTS substrate). The results showed that the half-life of TGFRt15-TGFRs was 12.66 hours in C57BL/6 mice.

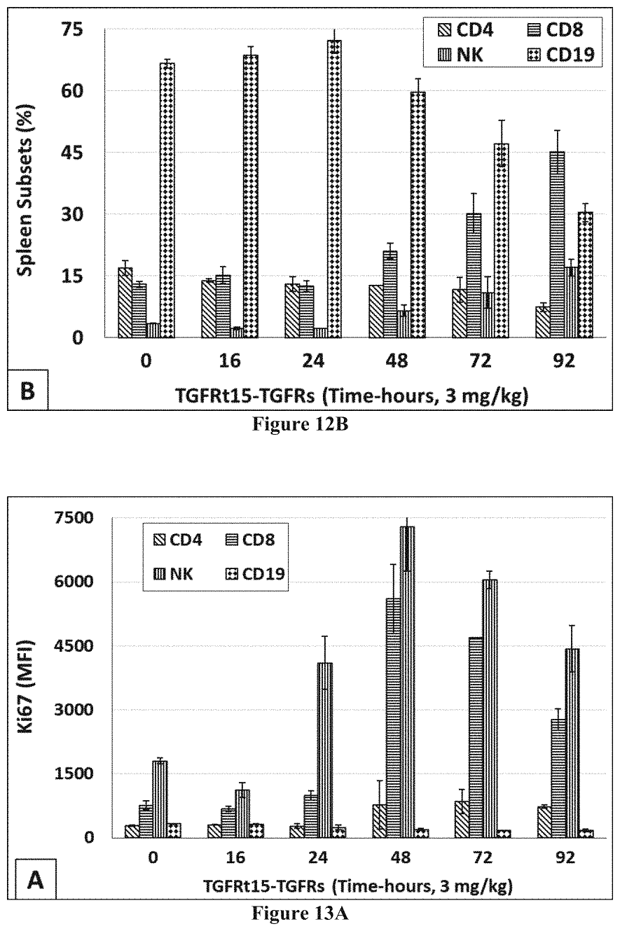

The mouse splenocytes were prepared in order to evaluate the immunostimulatory activity of TGFRt15-TGFRs over time in mice. As shown in FIG. 12A, the spleen weight in mice treated with TGFRt15-TGFRs increased 48 hours posttreatment and continued to increase over time. In addition, the percentages of CD4⁺ T cells, CD8⁺ T cells, NK cells, and CD19⁺ B cells present in the spleen of control-treated and TGFRt15-TGFRs-treated mice were evaluated. As shown in FIG. 12B, in the spleens of mice treated with TGFRt15-TGFRs, the percentages of CD8⁺ T cells and NK cells both increased at 48 hours after treatment and were higher and higher overtime after the single dose treatment. These results further demonstrate that TGFRt15-TGFRs is able to stimulate immune cells in the spleen, in particular CD8⁺ T cells and NK cells.

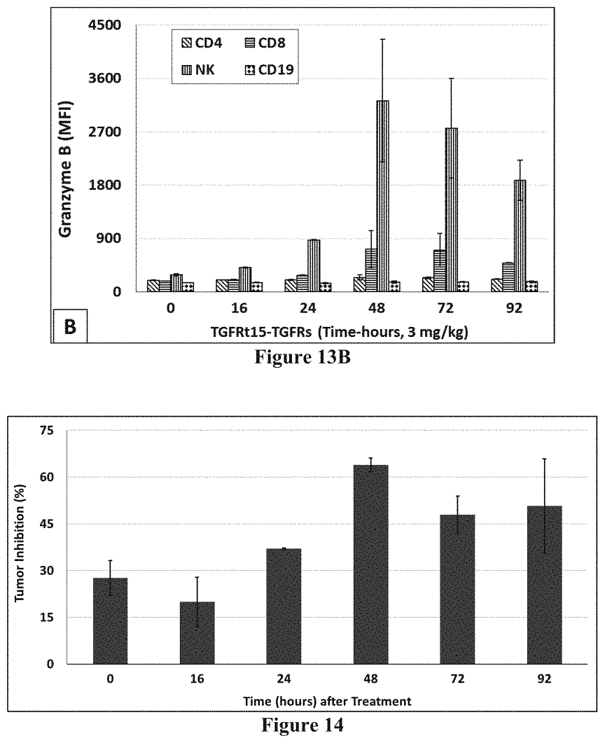

Furthermore, the dynamic proliferation of immune cells based on Ki67 expression of splenocytes and cytotoxicity potential based on granzyme B expression were evaluated in splenocytes isolated from mice following a single dose (3 mg/kg) of TGFRt15-TGFRs. As shown in FIG. 13A and 13B, in the spleens of mice treated with TGFRt15-TGFRs, the expression of Ki67 and granzyme B by NK cells increased at 24 hours after treatment and its expression of CD8⁺ T cells and NK cells both increased at 48 hours and later time points after the single dose treatment. These results demonstrate that TGFRt15-TGFRs not only increases the numbers of CD8⁺ T cells and NK cells but also enhance the cytotoxicity of these cells. The single dose treatment of TGFRt15-TGFRs led CD8⁺ T cells and NK cells to proliferate for at least 4 days.

The cytotoxicity of the splenocytes from TGFRt15-TGFRs-treated mice against tumor cells was also evaluated. Mouse Moloney leukemia cells (Yac-1) were labeled with CellTrace Violet and were used as tumor target cells. Splenocytes were prepared from TGFRt15-TGFRs (3 mg/kg)-treated mouse spleens at various time points post treatment and were used as effector cells. The target cells were mixed with effector cells at an E:T ratio=10:1 and incubated at 37° C. for 20 hours. Target cell viability was assessed by analysis of propidium iodide positive, violet-labeled Yac-1 cells using flow cytometry. Percentage of Yac-1 tumor inhibition was calculated using the formula, (1-[viable Yac-1 cell number in experimental sample]/[viable Yac-1 cell number in the sample without splenocytes])×100. As shown in FIG. 14, splenocytes from TGFRt15-TGFRs-treated mice had stronger cytotoxicity against Yac-1 cells than the control mouse splenocytes.

Tumor Size Analysis in Response to Chemotherapy and/or TGFRt15-TGFRs

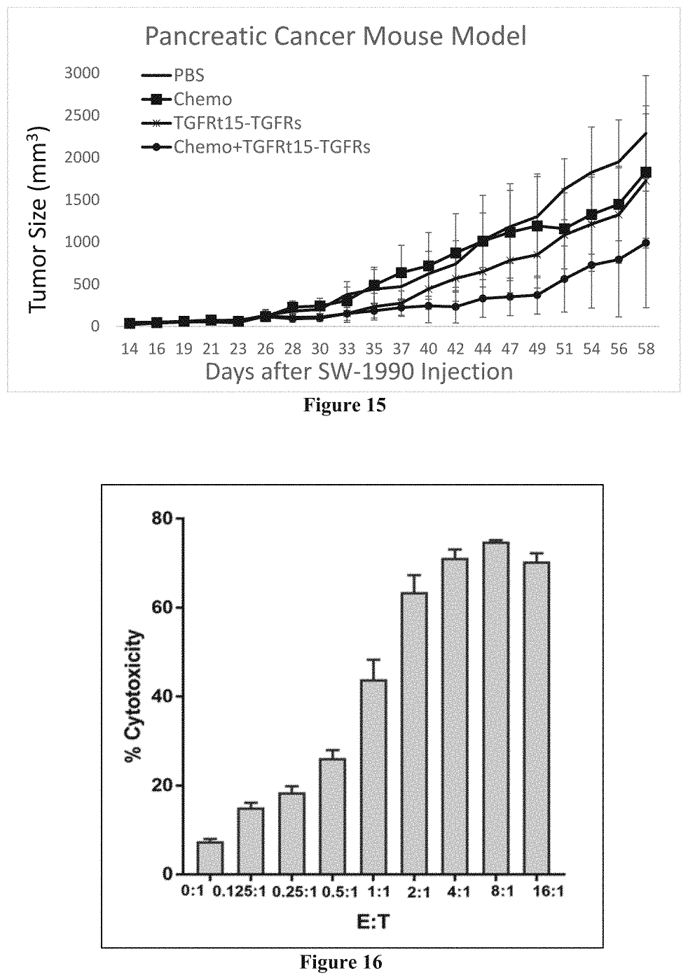

Pancreatic cancer cells (SW1990, ATCC® CRL-2172) were subcutaneously (s.c.) injected into C57BL/6 scid mice (The Jackson Laboratory, 001913, 2×10⁶ cells/mouse, in 1004 μL HBSS) to establish the pancreatic cancer mouse model. Two weeks after tumor cell injection, chemotherapy was initiated in these mice intraperitoneally with a combination of Abraxane (Celgene, 68817-134, 5 mg/kg, i.p.) and Gemcitabine (Sigma Aldrich, G6423, 40 mg/kg, i.p.), followed by immunotherapy with TGFRt15-TGFRs (3 mg/kg, s.c.) in 2 days. The procedure above was considered one treatment cycle and was repeated for another 3 cycles (1 cycle/week). Control groups were set up as the SW1990-injected mice that received PBS, chemotherapy (Gemcitabine and Abraxane), or TGFRt15-TGFRs alone. Along with the treatment cycles, tumor size of each animal was measured and recorded every other day, until the termination of the experiment 2 months after the SW1990 cells were injected. Measurement of the tumor volumes were analyzed by group and the results indicated that the animals receiving a combination of chemotherapy and TGFRt15-TGFRs had significantly smaller tumors comparing to the PBS group, whereas neither chemotherapy nor TGFRt15-TGFRs therapy alone work as sufficiently as the combination (FIG. 15).

In vitro Senescent B 16F10 Melanoma Model

Next, in vitro killing of senescent B16F10 melanoma cells by activated mouse NK cells was evaluated. B16F10 senescence cells (B16F10-SNC) cells were labelled with CellTrace violet and incubated for 16 hrs with different E:T ratio of in vitro 2t2-activated mouse NK cells (isolated from spleen of C57BL/6 mice injected with TGFRt15-TGFRs10 mg/kg for 4 days). The cells were trypsinized, washed and resuspended in complete media containing propidium iodide (PI) solution. The cytotoxicity was assessed by flow cytometry (FIG. 16).

### Example 3

### Stimulation of NK Cells In Vivo by TGFRt15-TGFRs

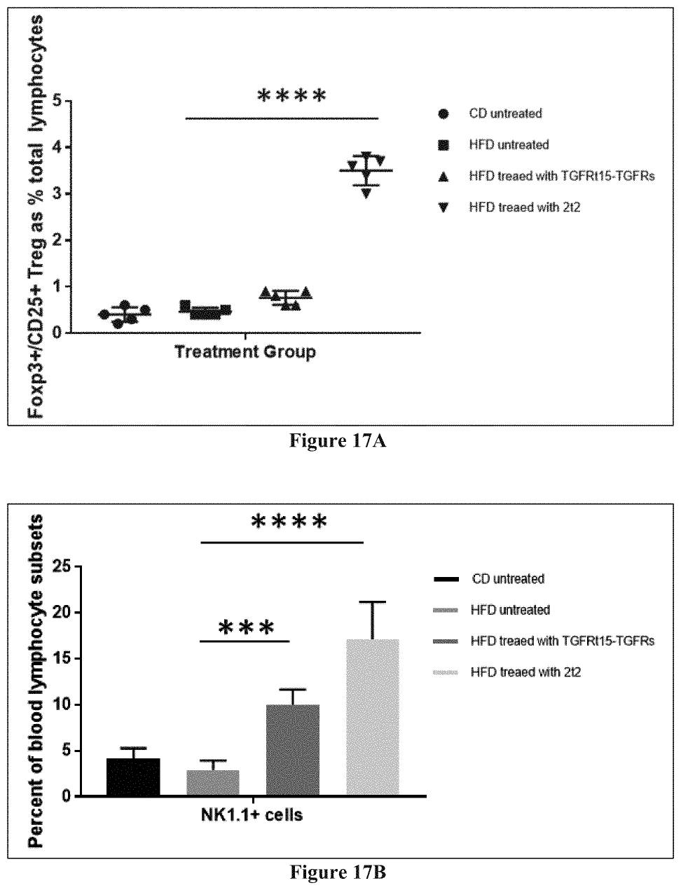

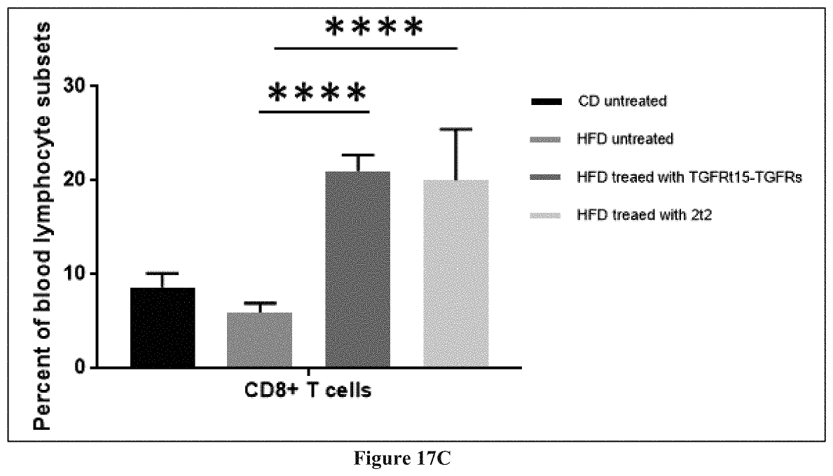

A set of experiments was performed to determine the effect of the TGFRt15-TGFRs construct on immune stimulation in ApoE^{-/-} mice fed with a Western diet. In these experiments, 6-week-old female B6.129P2-ApoE^{tmlUnc}/J mice (Jackson Laboratory) were fed with a Western diet containing 21% fat, 0.15% cholesterol, 34.1% sucrose, 19.5% casein, and 15% starch (TD88137, Envigo Laboratories). After 8-weeks of the Western diet, the mice were injected subcutaneously with TGFRt15-TGFRs at 3 mg/kg. Three days post treatment, mice were fasted for 16 hours and then blood samples were collected through retro-orbital venous plexus puncture. The blood was mixed with 10 μL 0.5 M EDTA, and 20 μL blood was taken for lymphocyte subsets analysis. The red blood cells were lysed with ACK (0.15 M NH₄Cl, 1.0 mM KHCO₃, 0.1 mM Na₂EDTA, pH 7.4) and the lymphocytes were stained with anti-mouse CD8a and anti-mouse NK1.1 antibodies for 30 minutes at 4 ° C. in FACS staining buffer (1% BSA in PBS). The cells were washed once and analyzed with a BD FACS Celesta. For Treg staining, ACK treated blood lymphocytes were stained with anti-mouse CD4 and anti-mouse CD25 antibodies for 30 minutes at 4 ° C. in FACS staining buffer. The cells were washed once and resuspended in fixation/permeabilization working solution and incubated at room temperature for 60 minutes. The cells were washed once and resuspended in permeabilization buffer. The samples were centrifuged at 300-400 x g for 5 minutes at room temperature and the supernatant was then discarded. The cell pellet was resuspended in residual volume and the volume adjusted to about 100 μL with 1 x permeabilization buffer. Anti-Foxp3 antibody was added to the cells, and the cells were incubated for 30 minutes at room temperature. Permeabilization buffer (200 μL) was added to the cells, and the cells were centrifuged at 300-400 x g for 5 minutes at room temperature. The cells were resuspended in flow cytometry staining buffer and analyzed on a flow cytometer. FIGS. 17A-17C show that treatment with TGFRt15-TGFRs increased the percentage of NK cells and CD8⁺ T cells in ApoE^{-/-} mice fed with Western diet.

### Example 4

### Induction of Proliferation of Immune Cells In Vivo

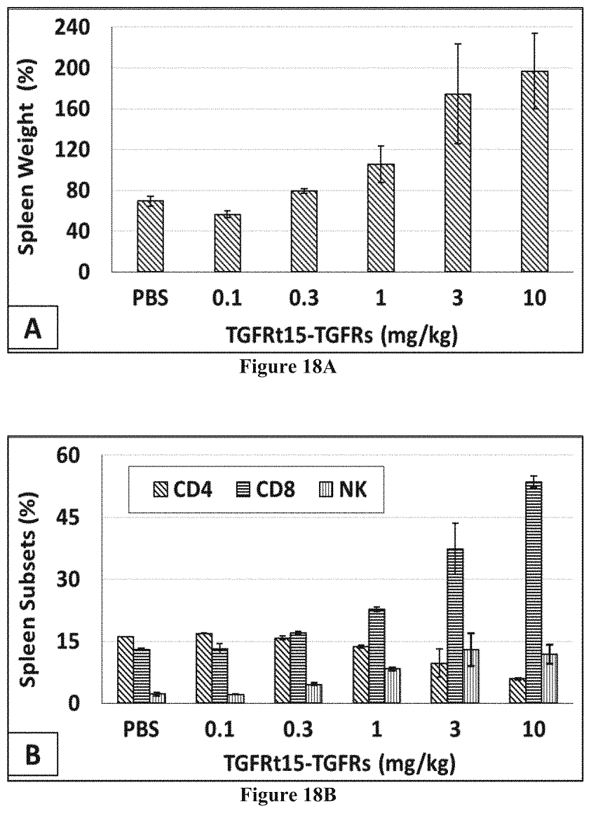

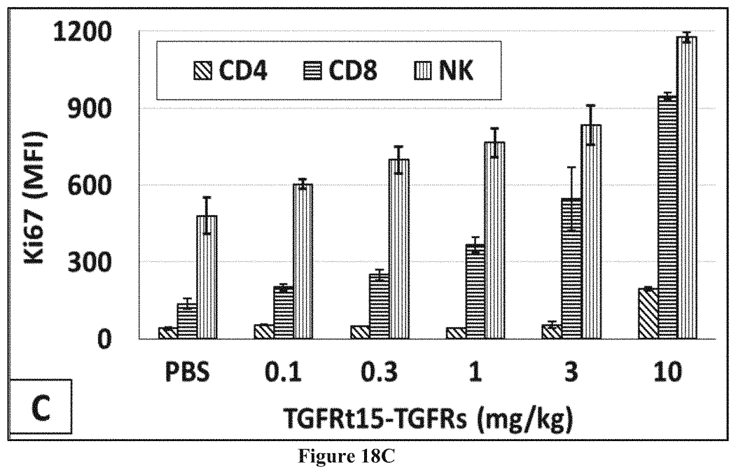

A set of experiments was performed to determine the effect of the TGFRt15-TGFRs construct on immune stimulation in C57BL/6 mice. In these experiments, C57BL/6 mice were subcutaneously treated with control solution (PBS) or TGFRt15-TGFRs at 0.1, 0.3, 1, 3, and 10 mg/kg. The treated mice were euthanized 4 days post-treatment. Spleen weight was measured and splenocyte suspensions were prepared. The splenocyte suspensions were stained with conjugated anti-CD4, anti-CD8, and anti-NK1.1 (NK) antibodies. The cells were additionally stained for proliferation marker Ki67. FIG. 18A shows that spleen weight in mice treated with TGFRt15-TGFRs increased with increasing dosage of TGFRt15-TGFRs. Additionally, spleen weight in mice treated with 1 mg/kg, 3 mg/kg, and 10 mg/kg of TGFRt15-TGFRs was higher as compared to mice treated with just the control solution. The percentages of CD8⁺ T cells and NK cells both increased with increasing dosage of TGFRt15-TGFRs (FIG. 18B). Finally, TGFRt15-TGFRs significantly upregulated expression of cell proliferation marker Ki67 in both CD8⁺ T cells and NK cells at all doses of TGFRt15-TGFRs tested (FIG. 18C). These results demonstrate that TGFRt15-TGFRs treatment induced proliferation of both CD8⁺ T cells and NK cells in C57BL/6 mice.

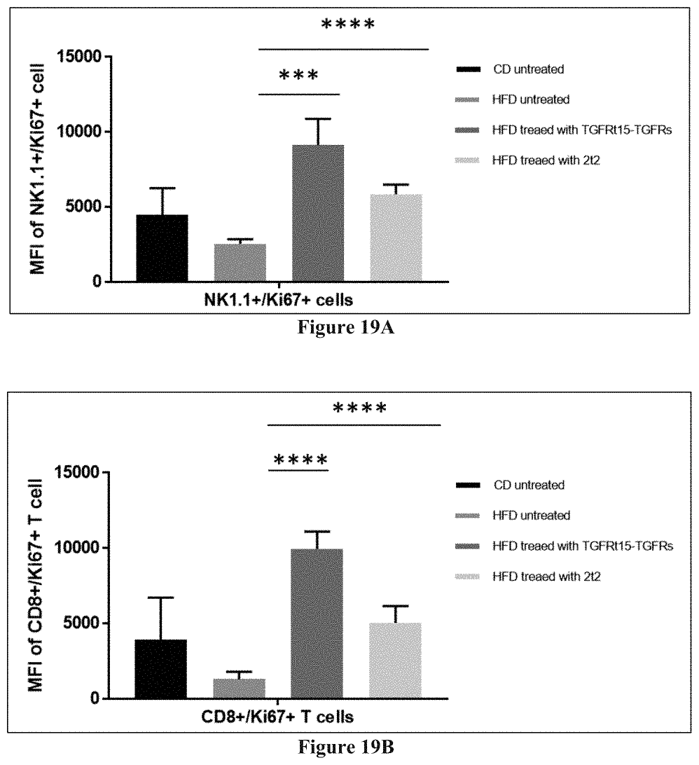

A set of experiments was performed to determine the effect of the TGFRt15-TGFRs construct on immune stimulation in ApoE^{-/-} mice fed with a Western diet. In these experiments, 6-week-old female B6.129P2-ApoE^{tmlUnc}/J mice (Jackson Laboratory) were fed with a Western diet containing 21% fat, 0.15% cholesterol, 34.1% sucrose, 19.5% casein, and 15% starch (TD88137, Envigo Laboratories). After 8-week of the Western diet, the mice were injected subcutaneously with TGFRt15-TGFRs at 3 mg/kg. Three days post-treatment, the mice were fasted for 16 hours and then blood samples were collected through retro-orbital venous plexus puncture. The blood was mixed with 10 μL 0.5 M EDTA and 20 μL blood was taken for lymphocyte subsets analysis. The red blood cells were lysed with ACK (0.15 M NH₄Cl, 1.0 mM KHCO₃, 0.1 mM Na₂EDTA, pH 7.4) and the lymphocytes were stained with anti-mouse CD8a and anti-mouse NK1.1 antibodies for 30 minutes at 4 ° C. in FACS staining buffer (1% BSA in PBS). The cells were washed once and resuspended in Fixation Buffer (BioLegend Cat# 420801) for 20 minutes at room temperature. The cells were centrifuged at 350 x g for 5 minutes, the fixed cells were resuspended in Intracellular Staining Permeabilization Wash Buffer (BioLegend Cat# 421002) and then centrifuged at 350 x g for 5 minutes. The cells were then stained with anti-Ki67 antibody for 20 minutes at RT. The cells were washed twice with Intracellular Staining Permeabilization Wash Buffer and centrifuged at 350 x g for 5 minutes. The cells were then resuspended in FACS staining buffer. Lymphocyte subsets were analyzed with a BD FACS Celesta. As described in FIG. 19A and 19B, treatment of ApoE^{-/-} mice with TGFRt15-TGFRs induced proliferation (Ki67-positive staining) in NK and CD8⁺ T cells.

### Example 5

### NK-Mediated Cytotoxicity Following Treatment with Multi-Chain Construct

A set of experiments was performed to determine if treatment of NK cells with TGFRt15-TGFRs enhanced cytotoxicity of NK cells. In these experiments, Human

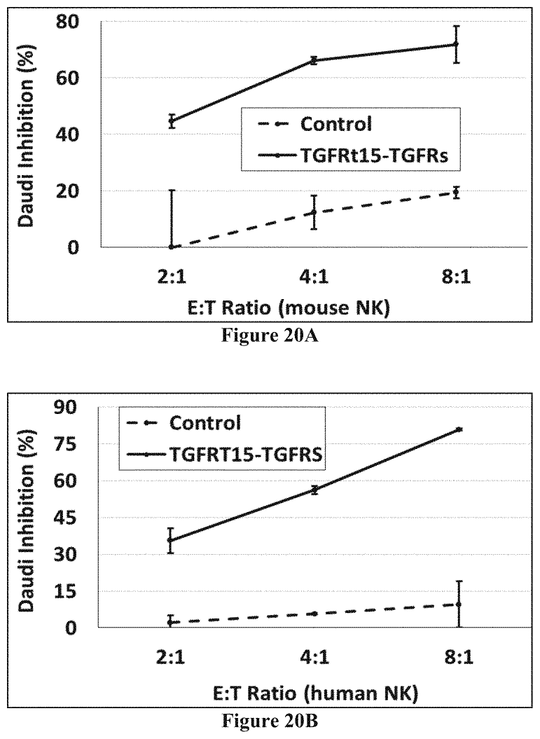

Daudi B lymphoma cells were labeled with CellTrace Violet (CTV) and used as tumor target cells. Mouse NK effector cells were isolated with NK1.1-positive selection using a magnetic cell sorting method (Miltenyi Biotec) of C57BL/6 female mouse spleens 4 days post TGFRt15-TGFRs subcutaneous treatment at 3 mg/kg. Human NK effector cells were isolated from peripheral blood mononuclear cells derived from human blood buffy coats with the RosetteSep/human NK cell reagent (Stemcell Technologies). The target cells (Human Daudi B lymphoma cells) were mixed with effector cells (either mouse NK effector cells or human NK effector cells) in the presence of 50 nM TGFRt15-TGFRs or in the absence of TGFRt15-TGFRs (control) and incubated at 37° C. for 44 hours for mouse NK cells and for 20 hours for human NK cells. Target cell (Daudi) viability was assessed by analysis of propidium iodide-positive, CTV-labeled cells using flow cytometry. The percentage of Daudi inhibition was calculated using the formula (1-viable tumor cell number in experimental sample/viable tumor cell number in the sample without NK cells)×100. FIG. 20 shows that mouse (FIG. 20A) and human (FIG. 20B) NK cells had significantly stronger cytotoxicity against Daudi B cells following NK cell activation with TGFRt15-TGFRs than in the absence of TGFRt15-TGFRs activation.

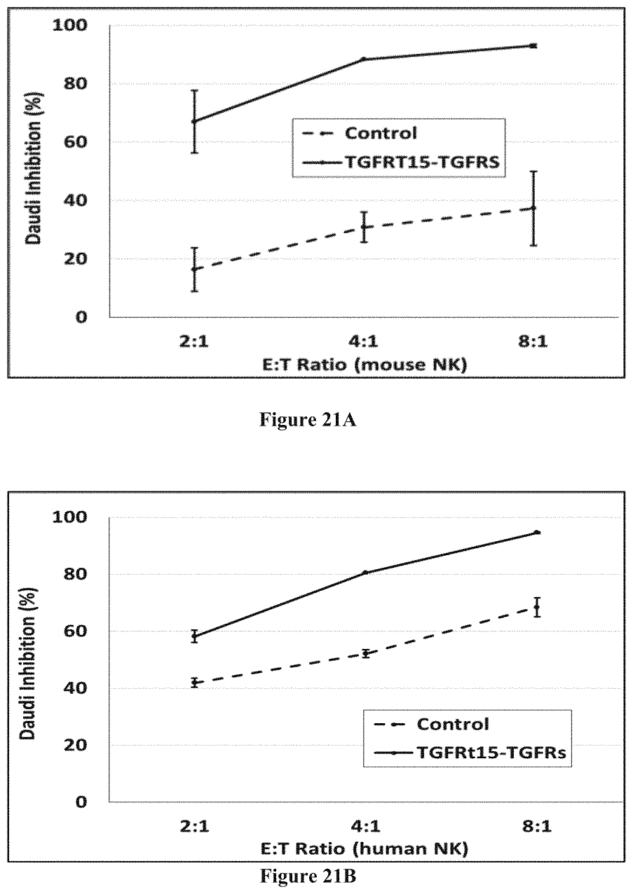

A set of experiments was performed to determine antibody-dependent cellular cytotoxicity (ADCC) of mouse and human NK cells following treatment with TGFRt15-TGFRs. In these experiments, human Daudi B lymphoma cells were labeled with CellTrace Violet (CTV) and used as tumor target cells. Mouse NK effector cells were isolated with NK1.1-positive selection using a magnetic cell sorting method (Miltenyi Biotec) of C57BL/6 female mouse spleens 4 days post-TGFRt15-TGFRs subcutaneous treatment at 3 mg/kg. Human NK effector cells were isolated from peripheral blood mononuclear cells derived from human blood buffy coats with the RosetteSep/human NK cell reagent (Stemcell Technologies). The target cells (Daudi B cells) were mixed with effector cells (either mouse NK effector cells or human NK effector cells) in the presence of anti-CD20 antibody (10 nM Rituximab, Genentech) and in the presence of 50 nM TGFRt15-TGFRs, or in the absence of TGFRt15-TGFRs (control) and incubated at 37° C. for 44 hours for mouse NK cells and for 20 hours for human NK cells. The Daudi B cells express the CD20 targets for the anti-CD20 antibody. Target cell viability was assessed after incubation by analysis of propidium iodide-positive, CTV-labeled target cells using flow cytometry. The percentage of Daudi inhibition was calculated using the formula (1-viable tumor cell number in experimental sample/viable tumor cell number in the sample without NK cells)×100. FIG. 21 shows that mouse NK cells (FIG. 21A) and human NK cells (FIG. 21B) had stronger ADCC activity against Daudi B cells following NK cell activation with TGFRt15-TGFRs than in the absence of TGFRt15-TGFRs activation.

### Example 6

### Treatment of Cancer

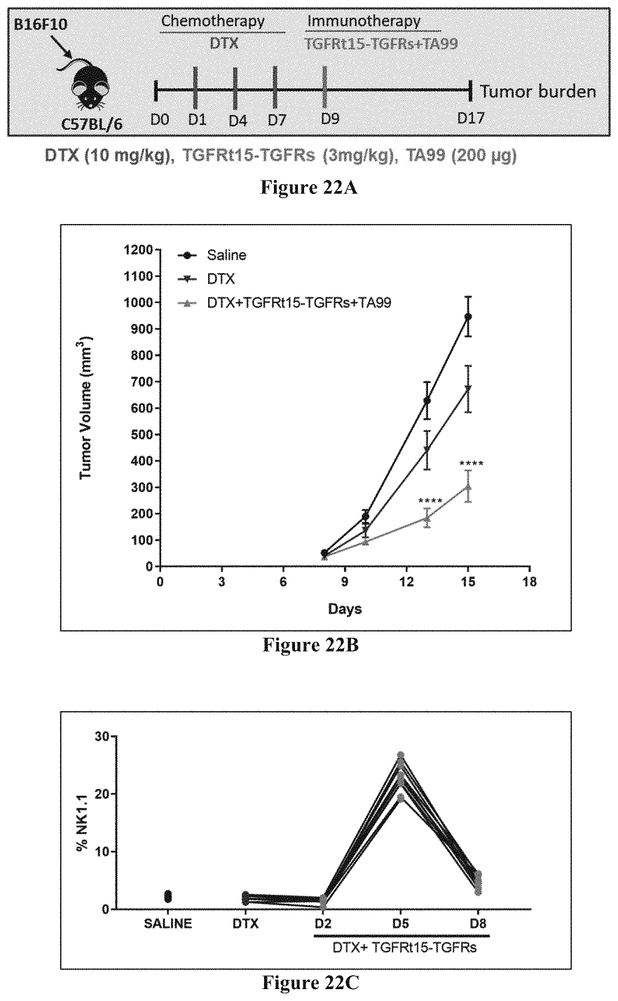

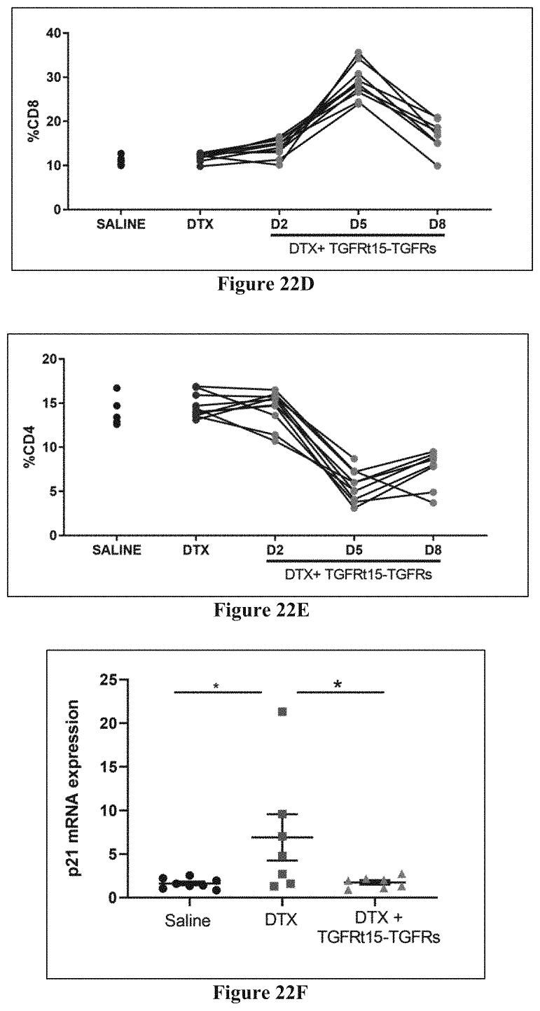

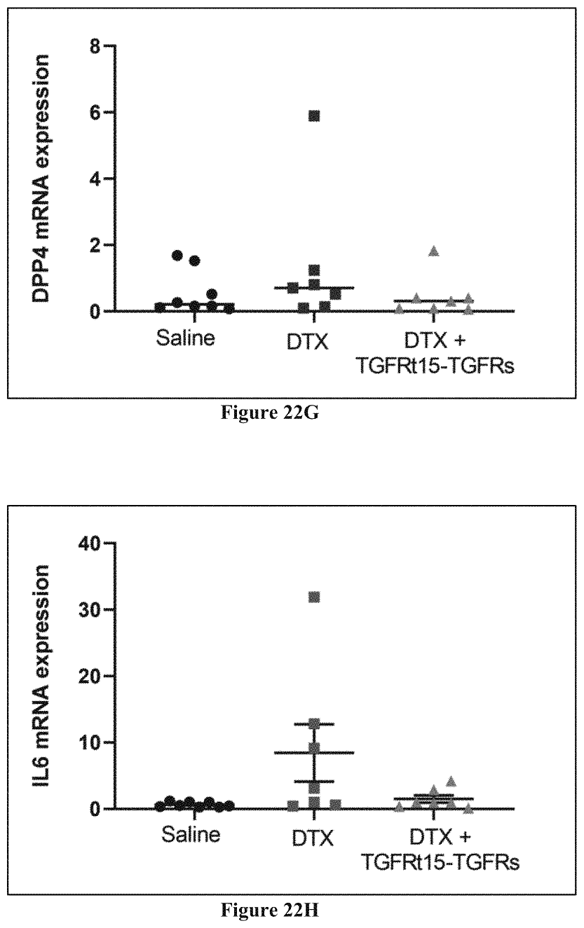

A set of experiments was performed to assess antitumor activity of TGFRt15-TGFRs plus anti-TRP1 antibody (TA99) in combination with chemotherapy in a melanoma mouse model. In these experiments, C57BL/6 mice were subcutaneously injected with 0.5×10⁶ B16F10 melanoma cells. The mice were treated with three doses of chemotherapy docetaxel (10 mg/kg) (DTX) on day 1, day 4, and day 7, followed by treatment with single dose of combination immunotherapy TGFRt15-TGFRs (3 mg/kg)+anti-TRP1 antibody TA99 (200 μg) on day 9. FIG. 22A shows a schematic of the treatement regimen. Tumor growth was monitored by caliper measurement, and tumor volume was calculated using the formula V=(L×W²)/2, where L is the largest tumor diameter and W is the perpendicular tumor diameter. FIG. 22B shows that treatment with DTX+TGFRt15-TGFRs+TA99 significantly reduced tumor growth compared to saline control and DTX treatment groups (N=10, ****p <0.001, Multiple t test analyses).

To assess immune cell subsets in the B16F10 tumor model, peripheral blood analysis was performed. In these experiments, C57BL/6 mice were injected with B16F10 cells and treated with DTX, DTX +TGFRt15-TGFRs +TA99, or saline. Blood was drawn from the submandibular vein of B16F10 tumor-bearing mice on days 2, 5, and 8 post-immunotherapy for the DTX +TGFRt15-TGFRs +TA99 group and day 11 post-tumor injection for the DTX and saline groups. RBCs were lysed in ACK lysis buffer and the lymphocytes were washed and stained with anti-NK1.1, anti-CD8, and anti-CD4 antibodies. The cells were analyzed by flow cytometry (Celesta-BD Bioscience). FIGS. 22C-22E show that DTX +TGFRt15-TGFRs +TA99 treatment induced an increase in the percentage of NK cells and CD8⁺T cells in the tumors compared to the saline and DTX treatment groups.

On day 17, total RNA was extracted from tumors of mice treated with saline, DTX or DTX+TGFRI-15-TGFRs+TA99 using Trizoi. Total RNA (1 μg) was used for cDNA synthesis using the QuantiTect Reverse Transcription Kit (Qiagen). Real-time PCR was carried out with CFX96 Detection System (Bio-Rad) using FAM-labeled predesigned primers for senescence cell markers, (F) p21 (G) DPP4 and (H) IL6. The housekeeping gene 18S ribosomal RNA was used as an internal control to normalize the variability in expression levels. The expression of each target mRNA relative to 18S rRNA was calculated based on Ct as 2 ^{-Δ(ΔCt)}, in which ΔCt=Ct_{target}−Ct_{18S}. The data is presented as fold-change as compared to saline control. FIG. 22F-22H show that DTX treatment induced an increase in senescent tumor cells that were subsequently reduced following treatment with TGFRt15-TGFRs+TA99 immunotherapy.

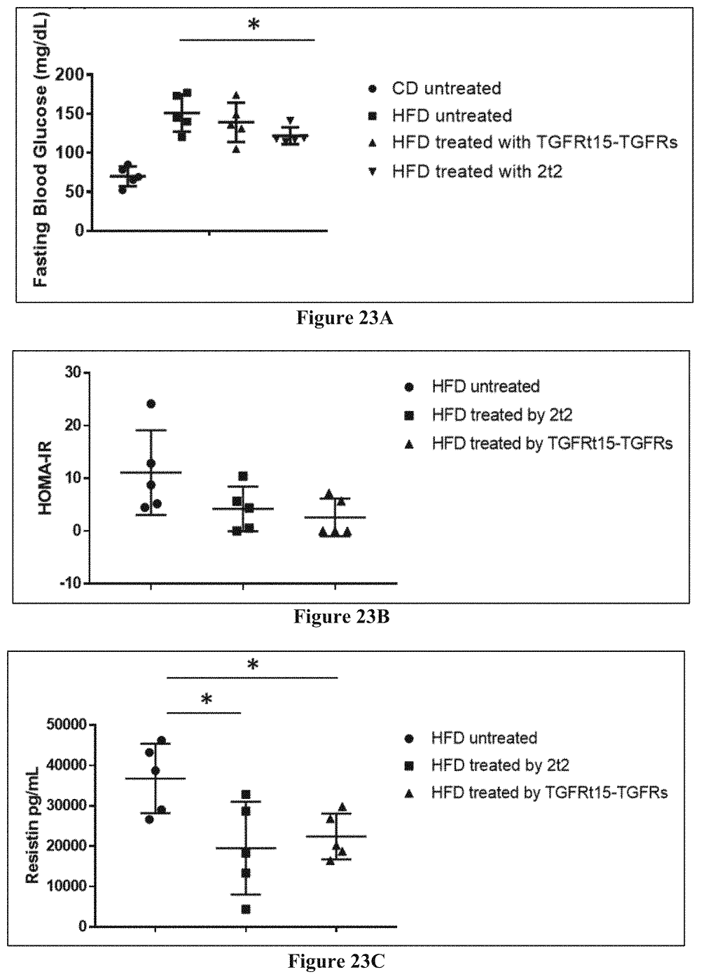

A set of experiments was performed to investigate amelioration of Western diet-induced hyperglycemia in ApoE^{-/-} mice by TGFRt15-TGFRs. In these experiments, 6-week-old female B6.129P2-ApoE^{tm1Unc}/J mice (Jackson Laboratory) were fed with a Western diet containing 21% fat, 0.15% cholesterol, 34.1% sucrose, 19.5% casein, and 15% starch (TD88137, Envigo Laboratories). After 8-weeks of the Western diet, the mice were injected subcutaneously with TGFRt15-TGFRs at 3 mg/kg. Three days post-treatment, the mice were fasted for 16 hours and then blood samples were collected through retro-orbital venous plexus puncture. Blood glucose was detected with a glucose meter (OneTouch UltraMini) and GenUltimated test strips using a drop of fresh blood. As shown in FIG. 23A, TGFRt15-TGFRs treatment reduced hyperglycemia induced by the Western diet. The plasma insulin and resistin levels were analyzed with Mouse Rat Metabolic Array by Eve Technologies. HOMA-IR was calculated using the following formula: homeostatic model assessment-insulin resistance =Glucose (mg/dL) * Insulin (mU/mL)/405. As shown in FIG. 23B, TGFRt15-TGFRs treatment reduced insulin resistance compared to the untreated group.

### Example 7

### Upregulation of CD44 Memory T Cells

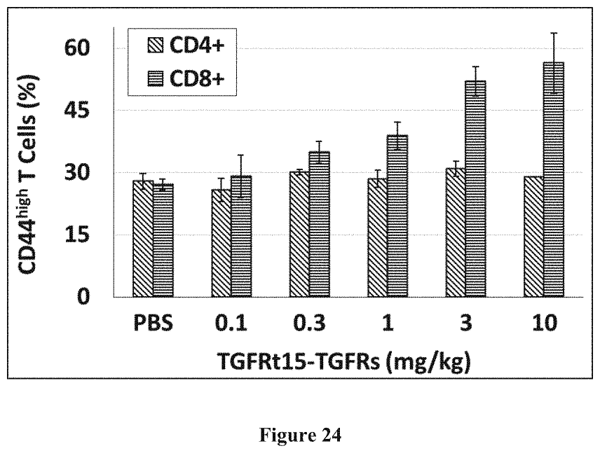

A set of experiments was performed to assess upregulation of CD44 memory T cells upon treatment with TGFRt15-TGFRs. In these experiments, C57BL/6 mice were subcutaneously treated with TGFRt15-TGFRs. The treated mice were euthanized and the single splenocyte suspensions were prepared 4 days (TGFRt15-TGFRs) following the treatment. The prepared splenocytes were stained with fluorochrome-conjugated anti-CD4, anti-CD8 and anti-CD44 antibodies and the percentages of CD44^{high} T cells in CD4⁺ T cells or CD8⁺ T cells were analyzed by flow cytometry. The results show that TGFRt15-TGFRs upregulated expression of the memory marker CD44 on CD4⁺ and CD8⁺ T cells (FIG. 24). These findings indicate that TGFRt15-TGFRs was able to induce mouse T cells to differentiate into memory T cells.

### Example 8

### Regulation of Transcriptomes in the Liver of db/db Mice Following Treatment with TGFRt15-TGFRs

Five-week-old male BKS.Cg-Dock7m +/+ Leprdb/J (db/db) mice were fed with standard chow diet and received drinking water ad libitum. At the age of six weeks, mice were randomly assigned to control and treatment groups (n=5/group). The treatment group received TGFRt15-TGFRs by subcutaneous injection at 3 mg/kg at 6 and 12 weeks of age, while control group received vehicle (PBS) only. At end of study (4-weeks post the 2^{nd} dose), mice were euthanized and livers were collected. The half of liver was homogenized with the TRIzol reagent (Invitrogen) and total tissue RNA was purified with RNeasy Mini Kit (Qiagen). Extracted RNA samples were quantified using Qubit 2.0Fluorometer (Life Technologies, Carlsbad, Calif., USA) and RNA integrity was checked using Agilent TapeStation 4200 (Agilent Technologies, Palo Alto, Calif., USA). RNA sequencing libraries were prepared using the NEBNext Ultra II RNA Library Prep Kit for Illumina following manufacturer's instructions (NEB, Ipswich, Mass., USA). Briefly, mRNAs were first enriched with Oligo(dT) beads. Enriched mRNAs were fragmented for 15 minutes at 94° C. First strand and second strand cDNAs were subsequently synthesized. cDNA fragments were end repaired and adenylated at 3′ends, and universal adapters were ligated to cDNA fragments, followed by index addition and library enrichment by limited-cycle PCR. The sequencing libraries were validated on the Agilent TapeStation (Agilent Technologies, Palo Alto, Calif., USA), and quantified by using Qubit 2.0 Fluorometer (Invitrogen, Carlsbad, Calif.) as well as by quantitative PCR (KAPA Biosystems, Wilmington, Mass., USA).

The sequencing libraries were clustered on 1 flowcell lane. After clustering, the flowcell was loaded on the Illumina HiSeq instrument (4000 or equivalent) according to manufacturer's instructions. The samples were sequenced using a 2×150bp Paired End

(PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification.

Sequence reads were trimmed to remove possible adapter sequences and nucleotides with poor quality using Trimmomatic v.0.36. The trimmed reads were mapped to the Mus musculus GRCm38 reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using featureCounts from the Subread package v.1.5.2. The hit counts were summarized and reported using the gene id feature in the annotation file. Only unique reads that fell within exon regions were counted. If a strand-specific library preparation was performed, the reads were strand-specifically counted. After extraction of gene hit counts, the gene hit counts table was used for downstream differential expression analysis. Using DESeq2, a comparison of gene expression between the treatment-specific groups of samples was performed. The Wald test was used to generate p-values and log2 fold changes. Genes with an adjusted p-value <0.05 and absolute log2 fold change >1 were called as differentially expressed genes for each comparison.

A gene ontology analysis was performed on the statistically significant set of genes by implementing the software GeneSCF v.1.1-p2. The mgi GO list was used to cluster the set of genes based on their biological processes and determine their statistical significance.

To estimate the expression levels of alternatively spliced transcripts, the splice variant hit counts were extracted from the RNA-seq reads mapped to the genome. Differentially spliced genes were identified for groups with more than one sample by testing for significant differences in read counts on exons (and junctions) of the genes using DEXSeq. For groups with only one sample, the exon hit count tables were provided.

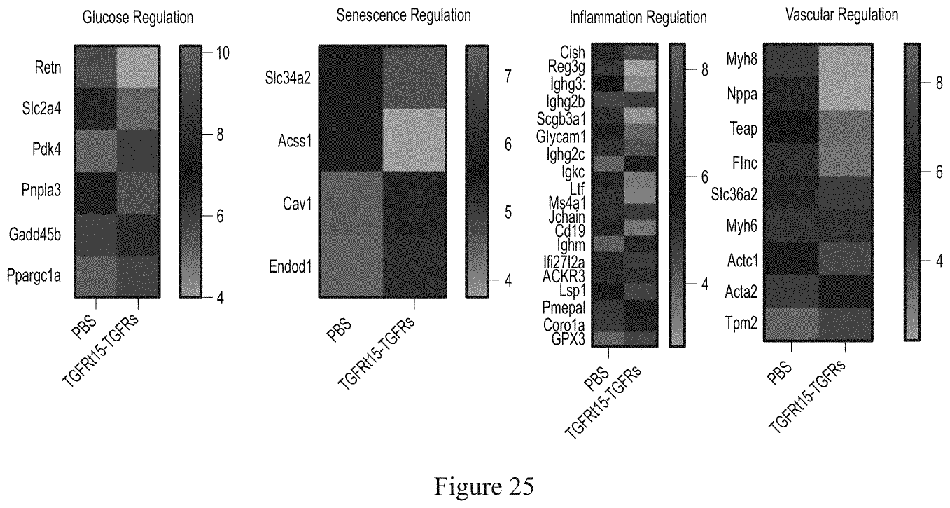

The significant genes downregulated or upregulated were divided into four groups according to the function. The heatmaps were constructed with GraphPad in accordance with gene functions. As shown in FIG. 25 and Tables 1 and 2, the six genes involved in glucose regulation were downregulated; the three genes related to senescence regulation were downregulated and one gene was upregulated; the nineteen genes involved in inflammation were mostly downregulated excepting one gene; the nine genes related to vascular regulation were downregulated.

Among six genes regulating glucose, four of them (Pdk4, Pnpla3, Gadd45b, and Ppargcla) were related to the gluconeogenesis. Downregulation of these four genes may cause the reduction of gluconeogenesis and therefore reduce the circulating glucose.

Downregulation of Retn was related to the reduction of insulin resistance. Downregulation of Slc2a4 may slow glucose transported to adipose tissue and striate muscle.

Downregulation of Cavl and Endodl along with upregulation of Slc34a2 promote cell proliferation and reduce senescence. Downregulation of Acssl may reduce glucose-independent acetate-mediated cell survival and tumor growth.

Downregulation of eighteen genes and upregulation of Cish are associated with downregulation of the cells and molecules involved in inflammatory responses. Downregulation of nine genes related to vascular regulation may reflect a different vascular environment in the liver changed by TGFRt15-TGFRs treatment.

These findings indicate that TGFRt15-TGFRs treatment suppresses gene expression related to glucose regulation, senescence, inflammation and vascular regulation in the liver of db/db mice.

TABLE 1
Regulation of transciptomes in the liver of db/db
mice following treatment with TGFRt15-TGFRs

| | | log2 Fold | | | | TGFRt15- |
|---|---|---|---|---|---|---|
| | Regulation | Change | Adj. Pval | Symbol | Control | TGFRs |
| Glucose | Down | −2.096289344 | 3.42E−02 | Retn | 5.61934746 | 3.974617084 |
| regulation | Down | −1.804543391 | 8.02E−03 | Slc2a4 | 6.648145442 | 5.116575093 |
| | Down | −1.346756214 | 9.80E−07 | Pdk4 | 10.15283618 | 8.812808986 |
| | Down | −1.319230698 | 1.82E−03 | Pnpla3 | 6.820864253 | 5.58617444 |
| | Down | −1.049951428 | 3.42E−02 | Gadd45b | 8.902818086 | 7.936747039 |
| | Down | −1.037624414 | 2.49E−03 | Ppargc1a | 9.94205237 | 8.965423502 |
| Senenscence | Up | 1.471480309 | 7.29E−02 | Slc34a2 | 5.768859731 | 7.064236232 |
| regulation | Down | −2.169290256 | 7.11E−02 | Acss1 | 5.443295282 | 3.721690488 |
| | Down | −1.610400236 | 4.18E−02 | Cav1 | 7.326547509 | 5.988279063 |
| | Down | −1.229365392 | 1.47E−02 | Endod1 | 7.446602038 | 6.192808898 |
| Inflammation | Up | 1.45707262 | 7.62E−05 | Cish | 6.321861131 | 7.704905566 |
| regulation | Down | −3.06345987 | 3.82E−02 | Reg3g | 4.932412574 | 2.829725656 |
| | Down | −2.886992871 | 8.02E−03 | lghg3 | 5.715159646 | 3.106258191 |
| | Down | −2.826778789 | 3.00E−14 | lghg2b | 7.437317137 | 4.709171503 |
| | Down | −2.791929301 | 3.76E−02 | Scgb3a1 | 5.023078231 | 3.024485524 |
| | Down | −2.604831835 | 8.38E−04 | Glycam1 | 6.095276793 | 3.867397849 |
| | Down | −2.562008178 | 5.67E−05 | lghg2c | 6.725829809 | 4.258477821 |
| | Down | −2.469457569 | 1.41E−11 | lgkc | 8.457634718 | 6.060534075 |
| | Down | −2.425601648 | 9.11E−02 | Ltf | 5.274677778 | 3.477435403 |
| | Down | −2.399351243 | 4.85E−02 | Ms4a1 | 5.017374783 | 3.304853422 |
| | Down | −2.047374161 | 8.63E−05 | Jchain | 6.660474662 | 4.797517709 |
| | Down | −2.039004438 | 7.47E−02 | Cd19 | 5.303977874 | 3.593322805 |
| | Down | −2.036901274 | 3.47E−07 | lghm | 8.299180551 | 6.411154088 |
| | Down | −1.909054054 | 1.65E−03 | Ifi27l2a | 6.420560573 | 4.753540666 |
| | Down | −1.706656106 | 4.71E−03 | ACKR3 | 6.465216788 | 4.982147331 |
| | Down | −1.467633284 | 3.97E−02 | Lsp1 | 5.884533344 | 4.544074275 |
| | Down | −1.11979923 | 4.71E−03 | Pmepa1 | 6.90863891 | 5.841136195 |
| | Down | −1.046058196 | 7.11E−02 | Coro1a | 7.076869734 | 6.124311808 |
| | Down | −1.038663712 | 2.19E−02 | GPX3 | 8.488888196 | 7.517625851 |
| Vascular | Down | −5.614042335 | 2.87E−02 | Myh8 | 4.469324747 | 2.214290342 |
| regulation | Down | −5.507281406 | 1.65E−03 | Nppa | 5.06772092 | 2.19472816 |
| | Down | −3.436742187 | 2.65E−03 | Tcap | 5.595187029 | 3.260187895 |
| | Down | −2.764903666 | 9.49E−02 | Flnc | 4.862671393 | 3.129487218 |
| | Down | −2.518127305 | 8.16E−03 | Slc36a2 | 6.244224409 | 4.402606708 |
| | Down | −2.317372389 | 3.00E−10 | Myh6 | 6.928245867 | 4.797511367 |
| | Down | −2.093059568 | 2.02E−02 | Actc1 | 5.784720054 | 4.123883209 |
| | Down | −1.550587332 | 2.25E−02 | Acta2 | 7.25724117 | 5.925475021 |
| | Down | −1.281068362 | 1.07E−03 | Tpm2 | 8.878431842 | 7.68227915 |

TABLE 2
Regulation of transcriptomes in the liver of db/db mice following treatment with TGFRt15-TGFRs

| Group | Regulation | Ensemble ID | Log2 Fold Change | Adj. Pval | Symbol | Control-6 | Control-7 |
|---|---|---|---|---|---|---|---|
| Glucose | Down | ENSMUSG00000027452 | −2.169290256 | 7.11E−02 | Acss1 | 5.855305552 | 5.084407867 |
| regulation | Down | ENSMUSG00000012705 | −2.096289344 | 3.42E−02 | Retn | 5.974583707 | 5.30069916 |
| | Down | ENSMUSG00000018566 | −1.804543391 | 8.02E−03 | Slc2a4 | 7.116144815 | 7.216009636 |
| | Down | ENSMUSG00000019577 | −1.346756214 | 9.80E−07 | Pdk4 | 9.972985223 | 9.92306323 |
| | Down | ENSMUSG00000041653 | −1.319230698 | 1.82E−03 | Pnpla3 | 6.916079316 | 7.041246469 |
| | Down | ENSMUSG00000015312 | −1.049951428 | 3.42E−02 | Gadd45b | 8.817325274 | 8.34108978 |
| | Down | ENSMUSG00000029167 | −1.037624414 | 2.49E−03 | Ppargcia | 9.941112425 | 9.427225266 |
| Senenscent cell | Up | ENSMUSG00000029188 | 1.471480309 | 7.29E−02 | Slc34a2 | 5.282818592 | 6.24485242 |
| regulation | Down | ENSMUSG00000007655 | −1.610400236 | 4.18E−02 | Cav1 | 7.962951204 | 7.996804652 |
| | Down | ENSMUSG00000037419 | −1.229365392 | 1.47E−02 | Endod1 | 7.497030385 | 7.216009636 |
| Inflammation | Up | ENSMUSG00000032578 | 1.45707262 | 7.62E−05 | Cish | 6.611948282 | 6.206382894 |
| regulation | Down | ENSMUSG00000030017 | −3.06345987 | 3.82E−02 | Reg3g | 5.167366003 | 5.724919575 |
| | Down | ENSMUSG00000076615 | −2.886992871 | 8.02E−03 | Ighg3; | 6.041689587 | 5.551619277 |
| | Down | ENSMUSG00000076613 | −2.826778789 | 3.00E−14 | Ighg2b | 7.58214335 | 7.456259351 |
| | Down | ENSMUSG00000064057 | −2.791929301 | 3.76E−02 | Scgb3a1 | 5.489230064 | 5.581987166 |
| | Down | ENSMUSG00000022491 | −2.604831835 | 8.38E−04 | Glycam1 | 6.536403573 | 6.581851777 |
| | Down | ENSMUSG00000076612 | −2.562008178 | 5.67E−05 | Ighg2c | 6.904107468 | 7.12612974 |
| | Down | ENSMUSG00000076609 | −2.469457569 | 1.41E−11 | Igkc | 8.738614728 | 8.835986045 |
| | Down | ENSMUSG00000032496 | −2.425601648 | 9.11E−02 | Ltf | 5.75222178 | 6.166859409 |
| | Down | ENSMUSG00000024673 | −2.399351243 | 4.85E−02 | Ms4a1 | 5.354953962 | 5.611728983 |
| | Down | ENSMUSG00000067149 | −2.047374161 | 8.63E−05 | Jchain | 6.85520118 | 7.084312295 |
| | Down | ENSMUSG00000030724 | −2.039004438 | 7.47E−02 | Cd19 | 5.75222178 | 5.354622897 |
| | Down | ENSMUSG00000076617 | −2.036901274 | 3.47E−07 | Ighm | 8.69063561 | 8.774860615 |
| | Down | ENSMUSG00000079017 | −1.909054054 | 1.65E−03 | Ifi27l2a | 6.765410892 | 6.854846927 |
| | Down | ENSMUSG00000044337 | −1.706656106 | 4.71E−03 | ACKR3 | 5.904203528 | 6.354464397 |
| | Down | ENSMUSG00000018819 | −1.467633284 | 3.97E−02 | Lsp1 | 5.904203528 | 5.996956972 |
| | Down | ENSMUSG00000038400 | −1.11979923 | 4.71E−03 | Pmepa1 | 6.87986155 | 6.804224856 |
| | Down | ENSMUSG00000030707 | −1.046058196 | 7.11E−02 | Coro1a | 7.319120952 | 7.406221275 |
| | Down | ENSMUSG00000018339 | −1.038663712 | 2.19E−02 | GPX3 | 8.731857489 | 8.879424465 |
| Vascular | Down | ENSMUSG00000055775 | −5.614042335 | 2.87E−02 | Myh8 | 5.456814408 | 5.951159834 |
| regulation | Down | ENSMUSG00000041616 | −5.507281406 | 1.65E−03 | Nppa | 4.641438054 | 5.206558526 |
| | Down | ENSMUSG00000007877 | −3.436742187 | 2.65E−03 | Tcap | 6.505042126 | 6.69731858 |
| | Down | ENSMUSG00000068699 | −2.764903666 | 9.49E−02 | Flnc | 5.641207544 | 5.778349917 |
| | Down | ENSMUSG00000020264 | −2.518127305 | 8.16E−03 | Slc36a2 | 7.126486166 | 7.216009636 |
| | Down | ENSMUSG00000040752 | −2.317372389 | 3.00E−10 | Myh6 | 6.985896265 | 7.186667466 |
| | Down | ENSMUSG00000068614 | −2.093059568 | 2.02E−02 | Actc1 | 6.319192998 | 6.336764352 |
| | Down | ENSMUSG00000035783 | −1.550587332 | 2.25E−02 | Acta2 | 7.380894129 | 8.089558631 |
| | Down | ENSMUSG00000028464 | −1.281068362 | 1.07E−03 | Tpm2 | 9.256882877 | 9.21109488 |

| Group | Regulation | Ensemble ID | Control-8 | C-Mean | X9218-11 | X9218-12 |
|---|---|---|---|---|---|---|
| Glucose | Down | ENSMUSG00000027452 | 4.390177426 | 5.443295282 | 1.168135905 | 2.998685697 |
| regulation | Down | ENSMUSG00000012705 | 4.582759514 | 5.61934 | 3.698843224 | 4.319824634 |
| | Down | ENSMUSG00000018566 | 5.612231874 | 5.64814 | 5.35547 | 4.951906104 |
| | Down | ENSMUSG00000019577 | 10.56246508 | 10.15283618 | 3.39860728 | 8.910281375 |
| | Down | ENSMUSG00000041653 | 6.505266974 | 6.820864253 | 5.390237181 | 5.670001969 |
| | Down | ENSMUSG00000015312 | 9.550039205 | 8.902818086 | 7.811522712 | 8.226223981 |
| | Down | ENSMUSG00000029167 | 10.45781942 | 9.94205237 | 9.13982396 | 8.842883134 |
| Senenscent cell | Up | ENSMUSG00000029188 | 5.77890818 | 5.768859731 | 6.805137717 | 7.980405385 |
| regulation | Down | ENSMUSG00000007655 | 6.019886672 | 7.326547509 | 6.390127612 | 5.90443626 |
| | Down | ENSMUSG00000037419 | 7.626766094 | 7.446602038 | 6.843270702 | 5.830444913 |
| Inflammation | Up | ENSMUSG00000032578 | 6.147252218 | 6.321861131 | 7.886482699 | 8.030833382 |
| regulation | Down | ENSMUSG00000030017 | 3.904952145 | 4.932412574 | 3.320548901 | 2 |
| | Down | ENSMUSG00000076615 | 5.552170073 | 5.715159646 | 4.320088877 | 2.998685697 |
| | Down | ENSMUSG00000076613 | 7.273548711 | 7.437317137 | 4.457550573 | 5.423879785 |
| | Down | ENSMUSG00000064057 | 3.998017464 | 5.023078231 | 3.168648016 | 2.321402517 |
| | Down | ENSMUSG00000022491 | 5.16757503 | 6.095276793 | 4.583046598 | 3.698619594 |
| | Down | ENSMUSG00000076612 | 6.147252218 | 6.725829809 | 4.521662724 | 5.085142674 |
| | Down | ENSMUSG00000076609 | 7.798303381 | 8.457634718 | 6.187651259 | 6.536642214 |
| | Down | ENSMUSG00000032496 | 3.904952145 | 5.274677778 | 3.457968795 | 2.80622845 |
| | Down | ENSMUSG00000024673 | 4.085441405 | 5.017374783 | 3.457968795 | 3.457758661 |
| | Down | ENSMUSG00000067149 | 6.041910512 | 6.660474662 | 5.167881281 | 4.904611708 |
| | Down | ENSMUSG00000030724 | 4.805088944 | 5.303977874 | 4.168136905 | 3.805476979 |
| | Down | ENSMUSG00000076617 | 7.432045428 | 8.299180551 | 6.670216194 | 6.456921454 |
| | Down | ENSMUSG00000079017 | 5.6414239 | 6.420560573 | 4.997988235 | 4.457280316 |
| | Down | ENSMUSG00000044337 | 7.136982438 | 6.465216788 | 4.952193823 | 4.951906104 |
| | Down | ENSMUSG00000018819 | 5.752439532 | 5.884533344 | 4.904898004 | 4.641647476 |
| | Down | ENSMUSG00000038400 | 7.041830324 | 6.90863891 | 5.805219897 | 5.698012376 |
| | Down | ENSMUSG00000030707 | 6.505266974 | 7.076869734 | 6.042234192 | 6.245436239 |
| | Down | ENSMUSG00000018339 | 7.855382635 | 8.488888196 | 7.711989961 | 7.23584466 |
| Vascular | Down | ENSMUSG00000055775 | 2 | 4.469324747 | 2.32146851 | 2.321402517 |
| regulation | Down | ENSMUSG00000041616 | 5.355166181 | 5.06772092 | 2 | 2 |
| | Down | ENSMUSG00000007877 | 3.583200381 | 5.595187029 | 3.168648016 | 2.80622845 |
| | Down | ENSMUSG00000068699 | 3.168456718 | 4.862671393 | 2.806369918 | 2.998685697 |
| | Down | ENSMUSG00000020264 | 4.390177426 | 6.244224409 | 4.246112511 | 4.641647476 |
| | Down | ENSMUSG00000040752 | 6.612173869 | 6.928245867 | 4.997988235 | 4.641647476 |
| | Down | ENSMUSG00000068614 | 4.698202813 | 5.784720054 | 4.246112511 | 3.805476979 |
| | Down | ENSMUSG00000035783 | 6.30127075 | 7.25724117 | 6.245749236 | 5.725489293 |
| | Down | ENSMUSG00000028464 | 8.167317769 | 8.878431842 | 7.663081872 | 7.473162775 |

| Group | Regulation | Ensemble ID | X9218-13 | X-Mean | Function |
|---|---|---|---|---|---|
| Glucose | Down | ENSMUSG00000027452 | 3.998243862 | 3.721699488 | Essential for glucose-independent |
| regulation | | | | | acetate-mediated cell survival and |
| | | | | | tumor growth. Glucose-starved |
| | | | | | melanoma cells are highly |
| | | | | | dependent on acetate to sustain |
| | | | | | ATP levels, cell viability and |
| | | | | | proliferation. Conversely, de- |
| | | | | | pletion of ACSS1 or ACSS2 reduced |
| | | | | | melanoma tumor growth in mice. |
| | Down | ENSMUSG00000012705 | 3.905178395 | 3.974617084 | Resistin is related to the |
| | | | | | pathogenesis of inflammation. |
| | Down | ENSMUSG00000018566 | 5.042342067 | 5.116575093 | Solute carrier family 2 member 4, |
| | | | | | also known as glucose transporter |
| | | | | | found primarily in adipose tissue |
| | | | | | and striated muscle (skeletal |
| | | | | | and cardiac). At the cell surface, |
| | | | | | GLUT4 permits the facilitated. |
| | | | | | diffusion of circulating glucose. |
| | Down | ENSMUSG00000019577 | 9.129538302 | 8.812808986 | Pyruvate dehydrogenase lipoamide |
| | | | | | kinase isozyme 4, is located in |
| | | | | | the matrix of the mitochondria |
| | | | | | and inhibits the pyruvate dehydro- |
| | | | | | genase complex by phosphorylating |
| | | | | | one of its subunits, reducing the |
| | | | | | conversion of pyruvate. Starvation |
| | | | | | and diabetes increase pyruvate |
| | | | | | dehydrogenase kinase-4 (PDK4) |
| | | | | | expression. |
| | Down | ENSMUSG00000041653 | 5.698284169 | 5.58617444 | Patatin-like phospholipase domain- |
| | | | | | containing protein 3 also known as |
| | | | | | adiponutrin (ADPN) is a triacylgly- |
| | | | | | cerol lipase that mediates tri- |
| | | | | | acylglycerolhydrosis in adipocytes |
| | | | | | and may also play a role in energy |
| | | | | | metabolism. In NASH biopsies, |
| | | | | | PNPLA3 significantly correlated |
| | | | | | with fibrosis stage and SMA levels |
| | | | | | independently of PNPLA3 genotype. |
| | | | | | In line, PNPLA3 expression was |
| | | | | | higher in SMA positive cells. Low |
| | | | | | glucose increased |
| | Down | ENSMUSG00000015312 | 7.772494424 | 7.936747039 | Growth arrest and DNA-damage- |
| | | | | | inducible, beta is involved in the |
| | | | | | regulation of growth and apoptosis. |
| | | | | | Both whole-body knockout (KO) mice |
| | | | | | and adenovirus-mediated knockdown |
| | | | | | (KD) mice of GADD45 exhibited de- |
| | | | | | creased hepatic gluconeiogenic gene |
| | | | | | expression concomitant with reduced |
| | | | | | blood glucose levels under fasting |
| | | | | | and HFD conditions, but showed a |
| | | | | | more pronounced effect in GADD45 KD |
| | | | | | mice. Further, in primary hepato- |
| | | | | | cytes, GADD45 KD reduced glucose |
| | | | | | output, whereas GADD45 overexpression |
| | | | | | increased it. Mechanistically, GADD45 |
| | | | | | did not affect Akt-mediated forkhead |
| | | | | | box protein O1 (FoxO1) phosphoryl- |
| | | | | | ation and forskolin-induced cAMP |
| | | | | | response element-binding protein |
| | | | | | (CREB) phosphorylation. Rather it |
| | | | | | increased FoxO1 transcriptional |
| | | | | | activity via enhanced protein |
| | | | | | stability of FoxO1. Further GADD45 |
| | | | | | colocalized and physically interacted |
| | | | | | with FoxO1. Additionally, GADD45 |
| | | | | | deficiency potentiated |
| | Down | ENSMUSG000000529167 | 8.913563411 | 8.965423502 | Peroxisome proliferation-activated |
| | | | | | receptor gamma coactivator 1-alpha |
| | | | | | (PGC-1a) is the master regulator of |
| | | | | | mitochondrial biogenesis and is also |
| | | | | | the primary regulator of |
| | | | | | gluconeogenesis in liver. The protein |
| | | | | | encoded by this gene is a |
| | | | | | transcriptional coactivator that |
| | | | | | regulates the genes involved in |
| | | | | | energy metabolism. This protein |
| | | | | | interacts with PPARgamma, which |
| | | | | | permits the interaction of this |
| | | | | | protein with multiple transcription |
| | | | | | factors. This protein can interact |
| | | | | | with, and regulate the activities of |
| | | | | | cAMP response element binding protein |
| | | | | | (CREB) and nuclear respiratory |
| | | | | | factors (NRFs). It provides a direct |
| | | | | | link between external physiological |
| | | | | | stimuli and the regulation of |
| | | | | | mitochondrial biogenesis, and is a |
| | | | | | major factor that regulates muscle |
| | | | | | fiber type determination. This |
| | | | | | protein may be also involved in |
| | | | | | controlling blood pressure, |
| | | | | | regulating cellular cholesterol |
| | | | | | homeostasis, and the development of |
| | | | | | obesity. PGC1A is key for insulin- |
| | | | | | mediated suppression of hepatic |
| | | | | | glucose production. While increased |
| | | | | | hepatic PGC1A levels are largely |
| | | | | | believed to negatively impact blood |
| | | | | | glucose control due to induction of |
| | | | | | enzymes that drive gluconeogenesis. |
| Senenscent cell | Up | ENSMUSG00000029188 | 6.407165595 | 7.064236232 | Solute carrier family 34m3mber 2, |
| regulation | | | | | (SLC34A2), a member of the SLC34 |
| | | | | | family, was initially isolated from a |
| | | | | | human small intestine. SLC34A2 is a |
| | | | | | multipass membrane protein and |
| | | | | | encodes a type 2b sodium-dependent |
| | | | | | phosphate transporter, NaPiIIb. It is |
| | | | | | known that SLC34A2 can mediate |
| | | | | | transporting inorganic phosphate into |
| | | | | | epithelial cells via sodium ion |
| | | | | | cotransport. Knockdown of SLC34A2 |
| | | | | | inhibits proliferation and migration |
| | | | | | by suppressing activation of the |
| | | | | | PI3K/AKT signaling pathway in |
| | | | | | hepatocellular carcinoma cells (HCC). |
| | Down | ENSMUSG00000007655 | 5.670273318 | 5.988279063 | Caveolin-1 is a scaffolding protein |
| | | | | | as the main component of the caveolae |
| | | | | | plasma membranes found in most cell |
| | | | | | types. The protein links integrin to |
| | | | | | promote cell cycle progression. |
| | | | | | Caveolin-1 plays a central role in |
| | | | | | the deveopment of a senescent |
| | | | | | phenotype and the regulation of both |
| | | | | | the anti-tumorigenicand proterties |
| | | | | | of cellular senescence. Caveolin-1 in |
| | | | | | expression controls on specific TGF-?1/ |
| | | | | | p53 responsive growth arrest genes. |
| | | | | | Indeed, up-regulation of caveolin-1 |
| | | | | | appears to stall cells in G0/G1 via |
| | | | | | activation of the p53/p21 cell cycle |
| | | | | | arrest pathway. The liver expresses |
| | | | | | deteactable CAV1 levels and, although |
| | | | | | some curvature has been described in |
| | | | | | the sinusoidal plasma membranes, |
| | | | | | hepatocytes do not form abundant |
| | Down | ENSMUSG00000037419 | 5.904711078 | 6.192808898 | Endonuclease domain containing 1 is a |
| | | | | | novel tumor suppressor in prostate |
| | | | | | cancer. Endonuclease domain |
| | | | | | containing 1 (ENDOD1) is a member of |
| | | | | | nucleases, which hydrolyze phosphodi- |
| | | | | | ester linkage in nucleic acids. It |
| | | | | | has been reported that nucleases |
| | | | | | participate in mutation avoidance, DNA |
| | | | | | repair |
| Inflammation | Up | ENSMUSG00000032578 | 7.197400617 | 7.704905566 | Cytokine-inducible SH2-containing |
| regulation | | | | | protein negatively regulates TCR |
| | | | | | signaling |
| | Down | ENSMUSG00000030017 | 3.168628066 | 2.829725656 | Regenerating islet-derived protein 3 |
| | | | | | gamma is one of several antimicrobial |
| | | | | | peptides. Among Reg family genes, |
| | | | | | Reg III? and III? were alternatively |
| | | | | | overexpressed in the colonic tissues |
| | | | | | of mice with DSS-induced colitis. The |
| | | | | | expression of STAT3-associated |
| | | | | | cytokines (IL-6, IL-17, and IL-22) |
| | | | | | was also significantly increased in |
| | | | | | those tissues, being significantly |
| | | | | | correlated with that of Reg III?/?. |
| | | | | | In the normal pancreas, Reg3? staining |
| | | | | | is absent or very minimally observed |
| | | | | | as small focal areas of |
| | Down | ENSMUSG00000076615 | 2 | 3.106258191 | |
| | Down | ENSMUSG00000076613 | 4.246084151 | 4.709171503 | |
| | Down | ENSMUSG00000064057 | 3.583406038 | 3.024485524 | Secretoglobin family 3 A member 1, |
| | | | | | SCGB3A1, also called UGRP2 or high |
| | | | | | in normal-1 (HIN-1), was described as |
| | | | | | a tumor suppressor in various human |
| | | | | | tumors including breast, prostate, |
| | | | | | lung, and pancreatic carcinomas. |
| | | | | | Ugrp2 gene is localized at chromosome |
| | | | | | 11B1 [3], a homologous region to |
| | | | | | 5q31-q35 in human, in which many genes |
| | | | | | encoding inflammatory cytokines such |
| | | | | | as interleukin-3, -4, -5, -13 |
| | | | | | and colony-stimulating factor 2 |
| | | | | | are located. These facts together |
| | | | | | with the sites of UGRP2 |
| | Down | ENSMUSG00000022491 | 3.320527354 | 3.867397849 | GlyCAM1 (Glycosylation-dependent cell |
| | | | | | adhesion molecule 1) is a proteoglycan |
| | | | | | ligand for L-selectin, modulating |
| | | | | | transendothelial migration of |
| | | | | | leukocytes. Existing evidence supports |
| | | | | | a role for GlyCAM1 as a negative |
| | | | | | regulator of extravasation. GlyCAM1 |
| | | | | | levels (protein and mRNA) decrease |
| | | | | | during acute antigen-primed |
| | | | | | inflammation and depletion of soluble |
| | | | | | L-selectin ligands (using L-selectin- |
| | | | | | IgG affinity columns) |
| | Down | ENSMUSG00000076612 | 3.168628066 | 4.258477821 | |
| | Down | ENSMUSG00000076609 | 5.457308753 | 6.060534075 | |
| | Down | ENSMUSG00000032496 | 4.168108965 | 3.477435403 | Lactoferrin (Lf) is a conserved iron- |
| | | | | | binding glycoprotein with |
| | | | | | antimicrobial activity. The |
| | | | | | infiltration of neutrophils into |
| | | | | | intestine tissues was changed |
| | | | | | similarly to Lf expression. It |
| | | | | | indicated that the variations of Lf |
| | | | | | expression were rather due to |
| | Down | ENSMUSG00000024673 | 2.998832811 | 3.304853422 | Membrane spanning 4-domain A1 encodes |
| | | | | | CD20. |
| | Down | ENSMUSG00000067149 | 4.320060138 | 4.797517709 | |
| | Down | ENSMUSG00000030724 | 2.806354532 | 3.593322805 | CD19 plays an essential role in |
| | | | | | regulating B-cell activation |
| | | | | | thresholds and thereby influences |
| | | | | | B-cell selection and differentiation. |
| | | | | | Altering CD19 surface expression in |
| | | | | | knockout or transgenic mice |
| | | | | | significantly changes B-cell |
| | | | | | development and function. CD19 |
| | | | | | overexpression results in B cells that |
| | | | | | are hyper-responsive to BCR triggering, |
| | | | | | leading to a lupus-like autoimmune |
| | | | | | disease with the production of anti- |
| | | | | | nuclear antibodies (ANAs) in the serum |
| | | | | | of transgenic mice (7). The |
| | Down | ENSMUSG00000076617 | 6.106324617 | 6.411154088 | |
| | Down | ENSMUSG00000079017 | 4.805353448 | 4.753540666 | Interferon alpha-inducible protein 27 |
| | | | | | like 2a is strongly up-regulated in |
| | | | | | the lung after influenza A infection. |
| | Down | ENSMUSG00000044337 | 5.042342067 | 4.982147331 | Atypical chemokine receptor 3 also |
| | | | | | known as CXCR-7 and GPR159 can bind |
| | | | | | the chemokines CXCL 12/SDF-1 and |
| | | | | | CXCL-11. ACKR3 functions primarily by |
| | | | | | sequestering the chemokine CXC-12 and |
| | | | | | endogenous opioid peptides. ACKR3 |
| | | | | | expression is usually faint or |
| | | | | | undetectable at steady state in the |
| | | | | | endothelium and in myeloid cells, but |
| | | | | | it can be upregulated during |
| | | | | | inflammation, for instance, by |
| | | | | | proinflammatory cytokines, such as |
| | | | | | interleukin8 (Singh and Lokeshwar, |
| | | | | | 2011) or IL-1b in vitro (Watanabe et |
| | | | | | al., 2010) and by environmental cues, |
| | | | | | such as lipopolysaccharide (Cao et |
| | | | | | al., 2016; Konrad et al., 2017; |
| | | | | | Ngamsri et al., 2017) or during |
| | | | | | infection by oncoviruses [reviewed |
| | | | | | in Freitas et al. (2014)]. Along |
| | | | | | this line, ACKR3 is highly |
| | | | | | upregulated during monocyte-to- |
| | | | | | macrophage differentiation in vitro, |
| | | | | | switching to a more pro-inflammatory |
| | | | | | cell phenotype (Ma et al., 2013; |
| | | | | | Chatterjee et al., 2015). Another |
| | | | | | example can be found during central |
| | | | | | nervous system inflammation, where |
| | | | | | ACKR3 is upregulated in endothelial |
| | | | | | cells of the blood-brain barrier(Cruz- |
| | | | | | Orengo et al., 2011). Antagonizing |
| | | | | | the scavenging activity of |
| | Down | ENSMUSG00000018819 | 4.085677346 | 4.544074275 | Lymphocyte-specific protein 1 may |
| | | | | | regulate neutrophil mobility, adhesion |
| | | | | | to fibrinogen matrix protein, and |
| | | | | | transendothelial migration. LSP |
| | | | | | (lymphocyte-specific protein) 1 as a |
| | | | | | critical regulator of actomyosin |
| | | | | | contractility in primary macrophages. |
| | | | | | LSP1 regulates adhesion and migration, |
| | | | | | including the |
| | Down | ENSMUSG00000038400 | 6.020176311 | 5.841136195 | Prostate transmembrane protein, |
| | | | | | andeogen induced 1 (PMEPA1), is |
| | | | | | induced by the TGF?? signalling, |
| | | | | | but meanwhile, it inhibits the |
| | | | | | phosphorylation of Smad2 and Smad3 to |
| | | | | | antagonize TGF?? signalling. PMEPA1 |
| | | | | | activates the bone morphogenetic |
| | | | | | proteins (BMP) signalling of TGF?? |
| | | | | | signalling resulting in |
| | Down | ENSMUSG00000030707 | 6.085264994 | 6.124311808 | Coro1A belongs to a family of |
| | | | | | evolutionary conserved actinbinding |
| | | | | | proteins that regulate actin |
| | | | | | cytoskeleton-dependent processes such |
| | | | | | as cytokinesis, cell polarization, |
| | | | | | migration, and phagocytosis. In the |
| | | | | | mammalian system, Coro1A is |
| | | | | | predominantly expressed in leukocytes |
| | | | | | and plays an important role, for |
| | | | | | example, in Ca2+ signaling in |
| | | | | | macrophages, TCR signaling, and |
| | | | | | lymphocyte |
| | Down | ENSMUSG00000018339 | 7.605042932 | 7.517625851 | Gltathione peroxidase-3 is an enzyme |
| | | | | | that functions in detoxification of |
| | | | | | hydrogen peroxide. GPX3 plays a |
| | | | | | pivotal role in arterial and venous |
| | | | | | thrombosis. GPX3 maintains the |
| | | | | | bioavailability of nitric oxide (NO) |
| | | | | | in the vascular system, and GPX3 |
| | | | | | deficiency leads to the decreased |
| | | | | | vascular bioavailability of NO, which |
| | | | | | attenuates its effect on platelet |
| | | | | | function and subsequently results in |
| | | | | | a prothrombotic state. Decreased GPX3 |
| Vascular | Down | ENSMUSG00000055775 | 2 | 2.214290342 | Myosin heavy chain 8 encodes a member |
| regulation | | | | | of the class II or conventional myosin |
| | | | | | heavy chain and functions in skeletal |
| | Down | ENSMUSG00000041616 | 2.584184479 | 2.19472816 | Natriuretic peptide A (Nppa) encoding |
| | | | | | arterial natruretic peptide (ANP) |
| | | | | | belongs to the natriuretic peptide |
| | | | | | family, is implicated in the decrease |
| | | | | | of blood preasure and inhibition of |
| | Down | ENSMUSG00000007877 | 3.80568722 | 3.260187895 | Telethinin, also known as Tcap, is |
| | | | | | expressed in cardiac and skeletal |
| | | | | | muscle at Z-disc and functions to |
| | | | | | regulate sarcomere |
| | Down | ENSMUSG00000068699 | 3.583406038 | 3.129487218 | Filamin C expression is restricted to |
| | | | | | striated muscles and localizes around |
| | | | | | the Z?disc, the sarcolemma, the |
| | | | | | myotendinous junction, and the |
| | | | | | intercalated discs. Its main role is |
| | | | | | maintaining the structural integrity |
| | | | | | of the sarcomere. This is through |
| | | | | | crosslinking actin filaments and the |
| | | | | | anchoring of sarcolemmal |
| | Down | ENSMUSG00000020264 | 4.320060138 | 4.402606708 | Solute carrier family 36 member 2 is |
| | | | | | a pH-dependent proton-coupled amino |
| | | | | | acid transporter that belongs to the |
| | | | | | amino acid auxin permease 1 protein |
| | | | | | family and primarily transports small |
| | | | | | amino acids such as glycine, alanine |
| | | | | | and proline.. SLC36A2 is expressed at |
| | | | | | the apical surface of the human renal |
| | | | | | proximal tubule where it functions in |
| | | | | | the reabsorption of glycine, proline |
| | | | | | and hydroxyproline. SLC36A2 also |
| | | | | | transports amino acid |
| | Down | ENSMUSG00000040752 | 4.752898391 | 4.797511367 | Myosin heavy chain 6 gene provides |
| | | | | | instruction for making a protein |
| | | | | | known as the cariac alpha-myosin |
| | | | | | heavy chain. |
| | Down | ENSMUSG00000068614 | 4.320060138 | 4.123883209 | Cardiac muscle alpha actin is the |
| | | | | | major protein of the thin filament |
| | | | | | in cardiac sarcomeres, which are |
| | | | | | responsible for muscle contraction and |
| | | | | | generation of force to support the |
| | | | | | pump |
| | Down | ENSMUSG000000035783 | 5.805186533 | 5.925475021 | smooth muscle actin or a-SMA, often |
| | | | | | used as a marker of myofibroblast |
| | | | | | formation. |
| | Down | ENSMUSG00000028464 | 7.910592804 | 7.68227915 | Tropomyosin beta chain is striated |
| | | | | | muscle-specific coiled coil dimer |
| | | | | | that functions to stabilize actin |
| | | | | | filaments and regulate |

| Group | Regulation | Ensemble ID | References | Potential Effects |
|---|---|---|---|---|
| Glucose | Down | ENSMUSG00000027452 | Lakhter, A J., et al., JBC VOL | Reduce glucose- |
| regulation | | | 291, NO. 42, pp. 21869-21879, | independent acetate |
| | | | Oct. 14, 2016 | related cell survival |
| | | | | and proliferation |
| | Down | ENSMUSG00000012705 | J. Biol. Chem. (2019) 294(30) | Increase circulating |
| | | | 11369-11381 | glucose by reducing |
| | | | | diffusion to fat and |
| | | | | muscle |
| | Down | ENSMUSG00000018566 | Diabetes 53: 899-910, 2004 | Increase glucose to |
| | | | | convert pyruvate reduce |
| | | | | circulating glucose by |
| | | | | consuming more. |
| | Down | ENSMUSG00000019577 | Liver International. 2020; | Reduce circulating |
| | | | 40: 1098-1110. | glucose by reducing |
| | | | | gluconeogenesis from |
| | | | | glycerol by triacylglycerol |
| | | | | hydrosis. |
| | Down | ENSMUSG00000041653 | Kim H. et al., GADD45 | Reduced liver |
| | | | regulates hepatic | gluconeogenesis and |
| | | | gluconeogenesis via | reduced blood glucose |
| | | | modulating the protein | levels. |
| | | | stability of FoxO1; | |
| | | | Biomolecules 2021; 9: 50. | |
| | Down | ENSMUSG00000015312 | Besse-Patin A et al., PGC1A | Reduce circulating |
| | | | regulates the IRS1:IRS2 ratio | glucose by reducing |
| | | | during fasting to influence | liver gluconeogenesis. |
| | | | hepatic metabolism | |
| | | | downstream of insulin PNAS | |
| | | | 2019; 116: 4284-4290. | |
| | Down | ENSMUSG00000029167 | | |
| Senenscent cell | Up | ENSMUSG00000029188 | Li, Y., et al., Knockdown of | Promote hepatocyte |
| regulation | | | SLC34A2 Inhibits Hepatocellular | proliferation and |
| | | | Carcinoma Cell Proliferation | migration |
| | | | and Invasion. Oncology | |
| | | | Research, Vol. 24, pp. | |
| | | | 511-519, 2016. | |
| | Down | ENSMUSG00000007655 | Pol A. et al., Non-caveolar | reduce senescent cells |
| | | | caveolins-duites outside the | |
| | | | caves J Cell Sci 2020; 133, | |
| | | | jcs241562; 2. Volonte D. et | |
| | | | al., Caveolin-1, a master | |
| | | | regulator of cellular | |
| | | | senescence; 3. Samarakoon, | |
| | | | R., et al., The TGF-?1/p53/ | |
| | | | PAI-1 Signaling Axis in | |
| | | | Vascular Senescence: Role of | |
| | | | Caveolin-1. | |
| | | | Biomolecules 2019, 9, 341; | |
| | Down | ENSMUSG00000037419 | Qiu, J., et al., Identification | reduce G0/G1 cell |
| | | | of endonuclease domaincontaining | cycle arrest |
| | | | 1 as a novel tumor suppressor | |
| | | | in prostate cancer. BMC Cancer | |
| | | | (2017) 17: 360 | |
| Inflammation | Up | ENSMUSG00000032578 | Palmer, DC., et al., Cish | inhibit T cell |
| regulation | | | actively silences TCR signaling | activation |
| | | | in CD8+ T cells to maintain | |
| | Down | ENSMUSG00000030017 | 1. Xu, X., et al., The Link | May reflect a reduction |
| | | | between Type III Reg and STAT3- | of inflammation |
| | | | Associated Cytokines in | |
| | | | Inflamed Colonic Tissues. | |
| | | | Mediators of Inflammation 2019; | |
| | | | 2019: 7859460. 2. Detection | |
| | | | of Reg3? by Immunohisto- | |
| | | | chemistry in Cerulein-Induced | |
| | | | Model of Acute Pancreatic Injury | |
| | | | in Mice and Rats Pancreas 48: 8, | |
| | Down | ENSMUSG00000076615 | | downregulation of B |
| | Down | ENSMUSG00000076613 | | downregulation of B |
| | Down | ENSMUSG00000064057 | 1. Xu, N., et al., Spatiotemporal | |
| | | | Expression of Three Secretoglobin | |
| | | | Proteins, SCGB1A1, SCGB3A1, and | |
| | | | SCGB3A2, in Mouse Airway | |
| | | | Epithelia. Journal of | |
| | | | Histochemistry & Cytochemistry | |
| | | | 2019, Vol. 67(6) 453-463; 2. | |
| | | | Yamada A. and Kimura, S. | reduce inflammation |
| | | | Induction of uteroglobin- | |
| | | | related protein 2 (Ugrp2) | |
| | | | expression by EGF and TGF?. | |
| | | | FEBS Lett. | |
| | Down | ENSMUSG00000022491 | Williams, P A., et al., | Increase lymphocyte |
| | | | GlyCAM1 negatively regulates | extravasation. |
| | | | monocyte entry into the optic | |
| | | | nerve head and contributes to | |
| | | | radiation-based protection in | |
| | | | glaucoma. Journal of | |
| | | | Neuroinflammation (2017) 14: 93 | |
| | Down | ENSMUSG00000076612 | | may relate to |
| | | | | downregulation of B |
| | Down | ENSMUSG00000076609 | | may relate to |
| | | | | downregulation of B |
| | Down | ENSMUSG00000032496 | Liang L., et al., Distribution | reduce neutrophil |
| | | | of Lactoferrin Is Related with | infiltration |
| | | | Dynamics of Neutrophils in | |
| | | | Bacterial Infected Mice | |
| | | | Intestine. Molecules 2020, 25, | |
| | | | 1496 | |
| | Down | ENSMUSG00000024673 | Cell Immunol 360, 2021, 104260 | may relate to |
| | | | | downregulation of B |
| | Down | ENSMUSG00000067149 | | may relate to |
| | | | | downregulation of B |
| | Down | ENSMUSG00000030724 | | may relate to |
| | | | | downregulation of B |
| | Down | ENSMUSG00000076617 | Morbach, H., et al., CD19 | cell function |
| | | | controls TLR9 responses in | may relate to |
| | | | human B cells. J Allergy Clin | downregulation of B |
| | | | Immunol. 2016 March; 137(3): | |
| | | | 889-898. | |
| | Down | ENSMUSG00000079017 | PLoS One 2014; 9(9): e106392 | may relate to |
| | | | | downregulation of |
| | Down | ENSMUSG00000044337 | Mol Pharmacol 96: 809-818, | ACKR3 is down that |
| | | | December 2019 | may be a sign of |
| | | | | inflammation is down |
| | Down | ENSMUSG00000018819 | NATURE COMMUNICATIONS \| | reduce inflammation |
| | | | (2018) 9: 515 | |
| | Down | ENSMUSG00000038400 | Zhang, L., et al., PMEPA1 | reflect a lower TGFB1 |
| | | | induces EMT via a non?canonical | levels |
| | | | TGF?? signalling in | |
| | | | colorectal cancer. J Cell Mol | |
| | | | Med. 2019; 23: 3603-3615. | |
| | Down | ENSMUSG00000030707 | Pick, R., et al., Coronin 1A, | reduce adoptive and |
| | | | a novel player in integrin | innate immunity |
| | | | biology, controls neutrophil | |
| | | | trafficking in innate immunity. | |
| | | | BLOOD, 17 Aug. 2017 | |
| | | | VOLUME 130, NUMBER 7 | |
| | Down | ENSMUSG00000018339 | Chen-Yu Chien, CY., et al., | increase ROS levels |
| | | | Glutathione peroxidase 3 gene | |
| | | | polymorphisms and the risk of | |
| | | | sudden sensorineural hearing | |
| | | | loss. Kaohsiung Journal of | |
| | | | Medical Sciences (2017) 33, | |
| | | | 359e364 | |
| Vascular | Down | ENSMUSG00000055775 | https://www.ncbi.nlm.nih.gov/ | muscle related |
| regulation | | | gtr/genes/4626/ | |
| | Down | ENSMUSG00000041616 | Handb Exp Pharmacol. 2009; | Increase blood |
| | | | (191): 341-366. | preasure and cardiac |
| | | | | hypertrophy/fibrosis |
| | Down | ENSMUSG00000007877 | https://en.wikipedia.org/wiki/ | muscle related |
| | | | Telethonin | |
| | Down | ENSMUSG00000068699 | Verdonschot, J A J., et al., A | muscle related |
| | | | mutation update for the FLNC | |
| | | | gene in myopathies and | |
| | | | cardiomyopathies. Human | |
| | | | Mutation. 2020; 41: 1091-1111. | |
| | Down | ENSMUSG00000020264 | Thwaites, D T., and Anderson, | reduce amino acid |
| | | | CMH., The SLC36 family of | derivatives |
| | | | proton-coupled amino acid | transportation |
| | | | transporters and their | |
| | | | potential role in drug | |
| | | | transport. British Journal of | |
| | | | Pharmacology (2011) 164 | |
| | | | 1802-1816 | |
| | Down | ENSMUSG00000040752 | https://medlineplus.gov/ | muscle related |
| | | | genetics/gene/myh6/ | |
| | Down | ENSMUSG00000068614 | https://en.wikipedia.org/ | Muscle related |
| | | | wiki/ACTC1 | |
| | Down | ENSMUSG00000035783 | https://en.wikipedia.org/wiki/ | muscle related |
| | | | ACTA2 | |
| | Down | ENSMUSG00000028464 | https://en.wikipedia.org/wiki/ | muscle related |
| | | | TPM2 | |

### Example 9

### RNA-seq Analysis of Differentially Expressed Genes Between the PBS (Control group) or TGFRt15-TGFRs (TGFRt15-TGFRs Group) In Aged Mice Liver

C57BL/6, 76-week-old mice were purchased from the Jackson Laboratory. Mice were housed in a temperature and light controlled environment. Mice were divided into two groups and treated subcutaneously with either PBS (PBS control group) or TGFRt15-TGFRs at a dosage of 3 mg/kg (TGFRt15-TGFRs group). At day 60 post treatment, mice were euthanized, and livers were harvested. Harvested livers were stored in liquid nitrogen in 1.7 mL Eppendorf tubes. Samples were homogenized by using homogenizer in 1 mL of Trizol (Thermo Fischer). Homogenized tissues were transferred in fresh Eppendorf tubes. Total RNA was extracted using RNeasy Mini Kit (Qiagen #74106) according to the manufacturer's instructions.

Library preparations, sequencing reactions and bioinformatic analysis were conducted at GENEWIZ, LLC. (South Plainfield, N.J., USA) as follows: Library preparation with poly A selection and HiSeq sequencing extracted RNA samples were quantified using Qubit 2.0 Fluorometer (Life Technologies, Carlsbad, Calif., USA) and

RNA integrity was checked using Agilent TapeStation 4200 (Agilent Technologies, Palo Alto, Calif., USA). RNA sequencing libraries were prepared using the NEBNext Ultra II RNA Library Prep Kit for Illumina following manufacturer's instructions (NEB, Ipswich, MA, USA). Briefly, mRNAs were first enriched with oligo(dT) beads. Enriched mRNAs were fragmented for 15 minutes at 94° C. First strand and second strand cDNAs were subsequently synthesized and cDNA fragments were end repaired and adenylated at 3′ ends. Universal adapters were ligated to cDNA fragments, followed by index addition and library enrichment by limited-cycle PCR. The sequencing libraries were validated on the Agilent TapeStation (Agilent Technologies, Palo Alto, Calif., USA), and quantified by using Qubit 2.0 Fluorometer (Invitrogen, Carlsbad, Calif.) as well as by quantitative PCR (KAPA Biosystems, Wilmington, Mass., USA). The sequencing libraries were clustered on 1 flowcell lane. After clustering, the flowcell was loaded on the Illumina HiSeq instrument (4000 or equivalent) according to manufacturer's instructions. The samples were sequenced using a 2×150bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification. Sequence reads were trimmed to remove possible adapter sequences and nucleotides with poor quality using Trimmomatic v.0.36. The trimmed reads were mapped to the Mus musculus GRCm38 reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using feature counts from the Subread package v.1.5.2. The hit counts were summarized and reported using the gene id feature in the annotation file. Only unique reads that fell within exon regions were counted. If a strand-specific library preparation was performed, the reads were strand-specifically counted. After extraction of gene hit counts, the gene hit counts table was used for downstream differential expression analysis. Using DESeq2, a comparison of gene expression between the treatment-specific groups of samples was performed. The Wald test was used to generate p-values and log2 fold changes. Genes with an adjusted p-value <0.05 and absolute log2 fold change >1 were called as differentially expressed genes for each comparison. A gene ontology analysis was performed on the statistically significant set of genes by implementing the software GeneSCF v.1.1-p2. The mgi GO list was used to cluster the set of genes based on their biological processes and determine their statistical significance. To estimate the expression levels of alternatively spliced transcripts, the splice variant hit counts were extracted from the RNA-seq reads mapped to the genome. Differentially spliced genes were identified for groups with more than one sample by testing for significant differences in read counts on exons (and junctions) of the genes using DEXSeq. For groups with only one sample, the exon hit count tables were provided.

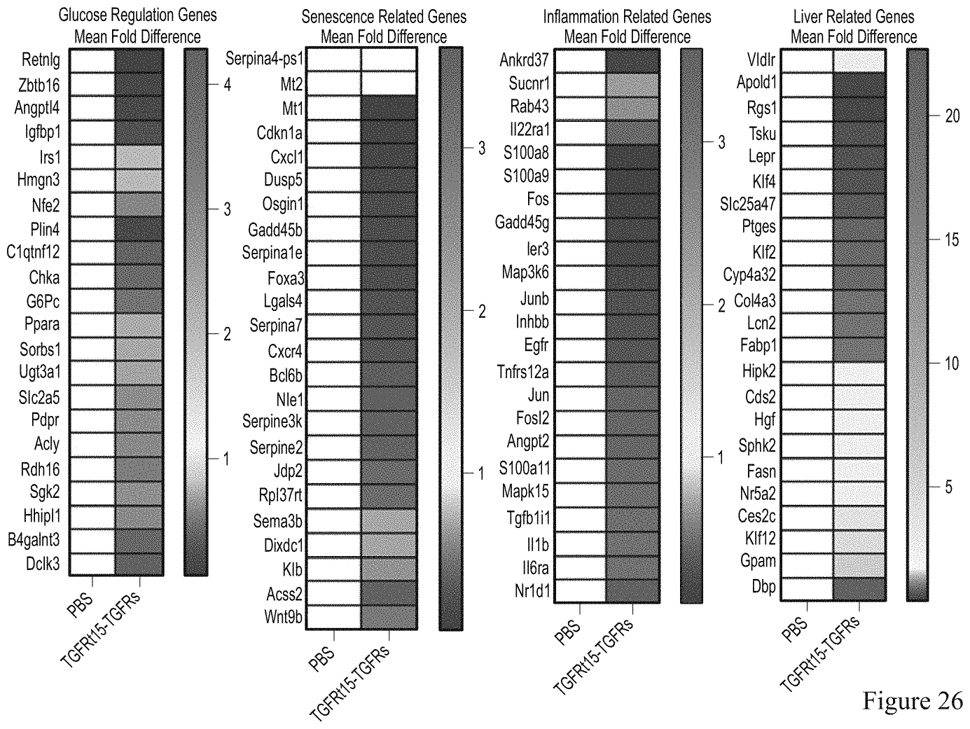

The significant genes downregulated or upregulated were divided into four groups according to the function. The mean fold change was calculated by dividing the experimental group by the mean the control group. The heatmaps were constructed with GraphPad in accordance with gene functions. As showed in FIG. 26 and Tables 3 and 4, most senescence and inflammation genes were downregulated in livers of the TGFRt15-TGFRs treated group compared to the PBS control group.

TABLE 3
RNA-seq analysis of differentially expressed genes between the PBS (Control
Group) or TGFRt15-TGFRs (TGFRt15-TGFRs group) in aged mice liver

| ID | log2FoldChange | pvalue | padj | agedliver92181tox | agedliver92182tox | agedliver92183tox |
|---|---|---|---|---|---|---|
| ENSMUSG00000000204 | −2.005879896 | 3.88E−05 | 0.000867087 | 8.974563618 | 11.65468178 | 11.93770314 |
| ENSMUSG00000000317 | −1.462090887 | 1.76E−06 | 7.11E−05 | 60.82759785 | 43.70505667 | 55.0970914 |
| ENSMUSG00000000686 | 1.151464201 | 2.84E−06 | 0.000104278 | 355.9910235 | 571.0794072 | 469.2435618 |
| ENSMUSG00000001227 | −1.099312542 | 1.49E−06 | 6.21E−05 | 100.7145473 | 67.98564371 | 92.74677053 |
| ENSMUSG00000001403 | −1.612087212 | 0.002110809 | 0.017174878 | 16.9519535 | 6.798564371 | 11.01941828 |
| ENSMUSG00000001983 | 1.013926416 | 1.20E−05 | 0.000332241 | 308.1266842 | 343.8131125 | 299.3608633 |
| ENSMUSG00000002233 | −1.270303057 | 1.05E−10 | 1.46E−08 | 133.6212805 | 104.892136 | 141.4158679 |
| ENSMUSG00000002250 | −1.142106413 | 9.77E−06 | 0.000282853 | 303.1408155 | 156.3669805 | 415.9830401 |
| ENSMUSG00000002289 | −2.433615292 | 5.35E−35 | 1.82E−31 | 340.0362437 | 320.5037489 | 528.0137926 |
| ENSMUSG00000002831 | −2.428567233 | 6.40E−14 | 1.90E−11 | 67.807814 | 71.87053764 | 51.42395197 |
| ENSMUSG00000003032 | −1.814709673 | 6.30E−10 | 7.35E−08 | 33.903907 | 28.16548097 | 44.99595798 |
| ENSMUSG00000003348 | 1.035062446 | 0.006673687 | 0.038727262 | 47.86433929 | 51.47484453 | 59.68851569 |
| ENSMUSG00000003500 | −1.169914769 | 9.39E−05 | 0.001721093 | 55.84172918 | 53.41729149 | 52.34223683 |
| ENSMUSG00000003541 | −2.579058324 | 2.05E−08 | 1.65E−06 | 9.971737353 | 11.65468178 | 11.01941828 |
| ENSMUSG00000003848 | −1.114660438 | 1.53E−05 | 0.000400378 | 168.5223613 | 191.3310259 | 135.9061588 |
| ENSMUSG00000004100 | −2.152000208 | 2.40E−05 | 0.000578522 | 177.4969249 | 181.6187911 | 165.2912742 |
| ENSMUSG00000004933 | −1.661199536 | 0.00641801 | 0.037574902 | 11.96608482 | 8.741011335 | 3.673139427 |
| ENSMUSG00000004951 | −1.245761383 | 7.16E−05 | 0.001407433 | 177.4969249 | 194.2446963 | 114.7856071 |
| ENSMUSG00000005148 | −1.641216558 | 0.00407345 | 0.027163637 | 8.974563618 | 3.884893927 | 9.182848567 |
| ENSMUSG00000005547 | 1.316837452 | 9.30E−15 | 3.02E−12 | 9129.125547 | 13948.71164 | 12101.15784 |
| ENSMUSG00000005580 | 1.4493211 | 2.75E−08 | 2.14E−06 | 248.2962601 | 371.00737 | 337.0105424 |
| ENSMUSG00000006050 | −1.042916307 | 2.32E−07 | 1.27E−05 | 992.1878666 | 1015.899762 | 869.6157593 |
| ENSMUSG00000006134 | 1.201348738 | 1.44E−09 | 1.52E−07 | 677.0809663 | 610.8995699 | 678.6125091 |
| ENSMUSG00000006517 | 1.182376585 | 1.39E−07 | 8.18E−06 | 2010.30225 | 1472.374798 | 1714.437827 |
| ENSMUSG00000006587 | 1.922052303 | 0.003206819 | 0.022932036 | 30.91238579 | 14.56835222 | 21.1205517 |
| ENSMUSG00000006711 | 1.246016282 | 4.07E−11 | 5.91E−09 | 435.7649223 | 410.8275327 | 410.4733309 |
| ENSMUSG00000006777 | 1.982368887 | 0.000106088 | 0.001901083 | 22.93499591 | 53.41729149 | 33.05825484 |
| ENSMUSG00000008153 | 1.444860417 | 1.72E−08 | 1.42E−06 | 278.2114721 | 339.9282186 | 185.4935411 |
| ENSMUSG00000009013 | −1.025154388 | 6.49E−07 | 3.04E−05 | 138.6071492 | 163.1655449 | 192.8398199 |
| ENSMUSG00000009633 | −1.150697898 | 7.41E−07 | 3.38E−05 | 1177.662181 | 1721.008009 | 1060.619009 |
| ENSMUSG00000014547 | 1.47853344 | 5.88E−06 | 0.00018548 | 76.78237762 | 107.8058065 | 108.3576131 |
| ENSMUSG00000014609 | 1.198939285 | 0.004891473 | 0.030982358 | 34.90108074 | 33.99282186 | 34.89482455 |
| ENSMUSG00000015224 | −1.47719251 | 7.85E−07 | 3.55E−05 | 57.83607665 | 125.2878291 | 79.89078253 |
| ENSMUSG00000015312 | −2.170653759 | 1.83E−07 | 1.04E−05 | 27.92086459 | 16.51079919 | 10.10113342 |
| ENSMUSG00000016128 | 1.194768611 | 1.81E−09 | 1.86E−07 | 486.6207828 | 535.1441384 | 631.7799814 |
| ENSMUSG00000016356 | −1.303561468 | 6.94E−06 | 0.00021464 | 50.8558605 | 59.24463238 | 46.83252769 |
| ENSMUSG00000017737 | −1.495519922 | 0.005185903 | 0.032307064 | 19.94347471 | 7.769787853 | 5.50970914 |
| ENSMUSG00000017868 | 1.520950393 | 2.42E−16 | 1.03E−13 | 1574.537328 | 1919.1376 | 1179.077756 |
| ENSMUSG00000018486 | 1.561119026 | 0.006908525 | 0.039768921 | 26.92369085 | 21.3669166 | 27.5485457 |
| ENSMUSG00000019082 | −1.214544808 | 1.93E−12 | 4.05E−10 | 2583.677148 | 3303.131061 | 2721.796315 |
| ENSMUSG00000019726 | 1.436923585 | 3.39E−10 | 4.31E−08 | 430.7790536 | 552.626161 | 627.1885571 |
| ENSMUSG00000019737 | −1.672592181 | 0.00414668 | 0.027410702 | 8.974563618 | 9.712234816 | 6.427993997 |
| ENSMUSG00000019883 | 1.383035463 | 1.73E−09 | 1.79E−07 | 790.7587721 | 685.683778 | 707.0793397 |
| ENSMUSG00000020018 | −1.224865075 | 5.55E−07 | 2.68E−05 | 76.78237762 | 100.0360186 | 117.5404617 |
| ENSMUSG00000020027 | −2.148540831 | 0.000201757 | 0.00309142 | 308.1266842 | 206.8706016 | 291.0962996 |
| ENSMUSG00000020091 | 1.323362559 | 0.005934886 | 0.035409378 | 1482.797344 | 1694.784975 | 1814.530877 |
| ENSMUSG00000020122 | −1.730853584 | 6.04E−21 | 5.49E−18 | 534.4851221 | 692.4823424 | 689.6319274 |
| ENSMUSG00000020335 | 1.099463556 | 0.002629472 | 0.020167667 | 41.88129688 | 88.38133683 | 80.80906739 |
| ENSMUSG00000020429 | −1.914789163 | 0.005628577 | 0.034083881 | 1082.930677 | 219.4965068 | 977.0550875 |
| ENSMUSG00000020441 | −1.319584205 | 1.17E−07 | 7.17E−06 | 96.72585232 | 114.6043708 | 87.23706139 |
| ENSMUSG00000020532 | 1.125678465 | 6.99E−06 | 0.000215157 | 6805.710743 | 2936.008585 | 5607.047335 |
| ENSMUSG00000020641 | −1.035196027 | 2.26E−05 | 0.00055792 | 307.1295105 | 181.6187911 | 139.5792982 |
| ENSMUSG00000020656 | −2.17958622 | 1.43E−08 | 1.21E−06 | 16.9519535 | 7.769787853 | 23.87540627 |
| ENSMUSG00000020681 | 1.270736177 | 0.003452779 | 0.024158824 | 30.91238579 | 35.93526882 | 52.34223683 |
| ENSMUSG00000020692 | −1.373008874 | 7.68E−06 | 0.000232298 | 53.84738171 | 65.07197327 | 76.21764311 |
| ENSMUSG00000020812 | −1.318469089 | 0.000692163 | 0.007551219 | 32.90673326 | 43.70505667 | 47.75081255 |
| ENSMUSG00000020889 | 1.857219295 | 5.65E−19 | 3.50E−16 | 1005.151125 | 1714.209445 | 1803.511459 |
| ENSMUSG00000020917 | 1.579449336 | 7.98E−12 | 1.40E−09 | 34685.69121 | 17873.42573 | 22782.64729 |
| ENSMUSG00000020948 | 1.248898073 | 0.003935164 | 0.026500655 | 67.807814 | 53.41729149 | 56.93366111 |
| ENSMUSG00000020961 | 1.570215029 | 7.42E−07 | 3.38E−05 | 116.669327 | 116.5468178 | 141.4158679 |
| ENSMUSG00000021250 | −3.196150425 | 1.40E−18 | 7.95E−16 | 14.95760603 | 8.741011335 | 25.71197599 |
| ENSMUSG00000021260 | 1.563177947 | 0.002093122 | 0.017081929 | 31.90955953 | 29.13670445 | 23.87540627 |
| ENSMUSG00000021416 | 1.171425747 | 1.31E−08 | 1.13E−06 | 910.4196203 | 675.9715432 | 629.0251268 |
| ENSMUSG00000021453 | −2.706070458 | 5.59E−23 | 6.28E−20 | 149.5760603 | 61.18707934 | 130.3964497 |
| ENSMUSG00000021611 | 1.027773708 | 1.21E−09 | 1.30E−07 | 416.8186214 | 423.453438 | 475.6715558 |
| ENSMUSG00000021670 | 1.206582161 | 2.43E−07 | 1.30E−05 | 4843.272832 | 4623.994996 | 5239.733392 |
| ENSMUSG00000021684 | −1.138260866 | 0.001182935 | 0.011115132 | 32.90673326 | 25.25181052 | 22.03883656 |
| ENSMUSG00000021773 | −1.294178405 | 0.002327317 | 0.01846284 | 17.94912724 | 12.62590526 | 22.03883656 |
| ENSMUSG00000021775 | 1.550962326 | 2.41E−10 | 3.16E−08 | 1168.687618 | 1405.360378 | 1119.38924 |
| ENSMUSG00000021804 | 1.339955479 | 0.00532128 | 0.032924815 | 26.92369085 | 35.93526882 | 37.64967912 |
| ENSMUSG00000021958 | −1.041099878 | 0.007096785 | 0.040595156 | 25.92651712 | 21.3669166 | 47.75081255 |
| ENSMUSG00000022383 | 1.183413475 | 3.19E−08 | 2.41E−06 | 1160.710228 | 1260.648079 | 1669.441869 |
| ENSMUSG00000022389 | 1.619123522 | 2.95E−16 | 1.22E−13 | 1357.153454 | 2316.368004 | 1561.084256 |
| ENSMUSG00000022408 | 1.528188008 | 2.54E−06 | 9.66E−05 | 74.78803015 | 107.8058065 | 82.6456371 |
| ENSMUSG00000022528 | −1.557702695 | 1.66E−19 | 1.13E−16 | 205.4177895 | 185.503685 | 197.4312442 |
| ENSMUSG00000022651 | −4.171544663 | 2.34E−09 | 2.36E−07 | 3.988694941 | 2.913670445 | 0 |
| ENSMUSG00000022704 | −1.016808687 | 0.000177103 | 0.002808323 | 94.73150485 | 108.7770299 | 100.0930494 |
| ENSMUSG00000022853 | 1.04479536 | 2.29E−11 | 3.59E−09 | 9525.00352 | 10073.52995 | 7460.146176 |
| ENSMUSG00000022883 | 1.330834067 | 1.00E−09 | 1.09E−07 | 280.2058196 | 336.0433246 | 355.3762395 |
| ENSMUSG00000022887 | 1.363326654 | 1.68E−07 | 9.74E−06 | 1196.608482 | 1241.22361 | 1538.127135 |
| ENSMUSG00000022911 | 1.024722285 | 3.96E−05 | 0.000880701 | 133.6212805 | 188.4173554 | 193.7581048 |
| ENSMUSG00000023034 | −2.875895339 | 0.000200077 | 0.003084527 | 58.83325038 | 34.96404534 | 22.95712142 |
| ENSMUSG00000023044 | −1.350199698 | 7.38E−12 | 1.31E−09 | 1875.683796 | 2461.080302 | 2315.914409 |
| ENSMUSG00000023052 | −1.334599319 | 0.004914148 | 0.031082668 | 13.96043229 | 11.65468178 | 17.44741228 |
| ENSMUSG00000023067 | −3.022867976 | 2.66E−57 | 3.63E−53 | 136.6128017 | 95.1799012 | 134.9878739 |
| ENSMUSG00000023073 | 1.225822592 | 2.02E−05 | 0.00050363 | 443.7423122 | 525.4319036 | 592.2937326 |
| ENSMUSG00000023341 | −1.14606456 | 1.95E−06 | 7.71E−05 | 89.74563618 | 99.06479513 | 85.40049167 |
| ENSMUSG00000023571 | −1.317554169 | 0.002212852 | 0.017761424 | 18.94630097 | 21.3669166 | 14.69255771 |
| ENSMUSG00000023800 | 1.312092825 | 2.45E−12 | 4.99E−10 | 459.697092 | 410.8275327 | 456.3875738 |
| ENSMUSG00000023905 | −1.475845141 | 9.44E−06 | 0.000275028 | 54.84455544 | 95.1799012 | 81.72735225 |
| ENSMUSG00000023927 | −1.218211099 | 0.003077602 | 0.022366209 | 27.92086459 | 26.223034 | 12.85598799 |
| ENSMUSG00000023968 | −1.267291327 | 0.008148531 | 0.04468095 | 10.96891109 | 11.65468178 | 15.61084256 |
| ENSMUSG00000024118 | −1.757053931 | 5.31E−18 | 2.79E−15 | 442.7451385 | 592.4463238 | 352.621385 |
| ENSMUSG00000024130 | 1.042830636 | 1.30E−05 | 0.000354612 | 2989.526858 | 2745.648783 | 3530.805274 |
| ENSMUSG00000024136 | −1.225506878 | 0.002002269 | 0.016518413 | 27.92086459 | 19.42446963 | 17.44741228 |
| ENSMUSG00000024190 | −1.579039564 | 2.17E−13 | 5.48E−11 | 281.2029934 | 502.12254 | 460.0607132 |
| ENSMUSG00000024236 | 1.389584652 | 9.49E−13 | 2.12E−10 | 1344.190195 | 1041.151572 | 1299.373072 |
| ENSMUSG00000024411 | 1.68012077 | 5.07E−05 | 0.001068968 | 63.81911906 | 48.56117408 | 35.81310941 |
| ENSMUSG00000024440 | 1.007637941 | 0.000827422 | 0.008613399 | 107.6947634 | 103.9209125 | 147.8438619 |
| ENSMUSG00000024665 | 1.18951247 | 3.11E−12 | 6.05E−10 | 9244.7977 | 8635.147975 | 9906.457034 |
| ENSMUSG00000024843 | −1.175648724 | 2.45E−11 | 3.79E−09 | 1421.969747 | 1016.870985 | 1263.559963 |
| ENSMUSG00000024887 | 1.067323547 | 1.55E−06 | 6.39E−05 | 398.8694941 | 436.0793433 | 565.6634717 |
| ENSMUSG00000024924 | 1.265415535 | 2.60E−12 | 5.20E−10 | 642.1798855 | 816.7989481 | 589.538878 |
| ENSMUSG00000024970 | −1.174479585 | 0.000322102 | 0.004379373 | 31.90955953 | 39.82016275 | 25.71197599 |
| ENSMUSG00000024978 | 2.527860907 | 4.51E−08 | 3.23E−06 | 10405.50793 | 5475.757989 | 8311.396238 |
| ENSMUSG00000025003 | 1.045766059 | 5.74E−07 | 2.75E−05 | 301.1464681 | 286.5109271 | 410.4733309 |
| ENSMUSG00000025006 | 1.229102652 | 2.44E−06 | 9.41E−05 | 288.1832095 | 294.2807149 | 416.9013249 |
| ENSMUSG00000025153 | 1.113433094 | 9.44E−07 | 4.18E−05 | 85604.37365 | 41744.15646 | 57005.28733 |
| ENSMUSG00000025161 | −1.470478397 | 0.001962044 | 0.016285081 | 18.94630097 | 9.712234816 | 14.69255771 |
| ENSMUSG00000025240 | 1.333245056 | 1.72E−06 | 6.99E−05 | 553.4314231 | 682.7701076 | 696.0599214 |
| ENSMUSG00000025323 | 1.252220218 | 7.14E−07 | 3.28E−05 | 138.6071492 | 127.2302761 | 172.6375531 |
| ENSMUSG00000025402 | −1.340941683 | 6.59E−15 | 2.25E−12 | 217.3838743 | 245.7195409 | 214.8786565 |
| ENSMUSG00000025429 | 1.693186246 | 2.17E−13 | 5.48E−11 | 624.2307583 | 540.0002558 | 651.9822483 |
| ENSMUSG00000025450 | −1.257708301 | 2.83E−05 | 0.000669372 | 30.91238579 | 62.15830282 | 55.0970914 |
| ENSMUSG00000025997 | −1.284615532 | 0.009041141 | 0.048049118 | 26.92369085 | 9.712234816 | 12.85598799 |
| ENSMUSG00000026020 | −1.156643623 | 9.59E−08 | 6.11E−06 | 250.2906076 | 294.2807149 | 269.9757479 |
| ENSMUSG00000026249 | −1.326755053 | 0.000451208 | 0.005606296 | 36.89542821 | 50.50362104 | 26.63026084 |
| ENSMUSG00000026358 | −2.793399328 | 5.66E−13 | 1.33E−10 | 12.96325856 | 11.65468178 | 12.85598799 |
| ENSMUSG00000026398 | 1.137826301 | 6.48E−12 | 1.16E−09 | 1403.023446 | 1667.590718 | 1539.04542 |
| ENSMUSG00000026471 | 1.000583106 | 1.46E−05 | 0.000384933 | 269.2369085 | 237.949753 | 315.8899907 |
| ENSMUSG00000026475 | −1.56210025 | 0.004964709 | 0.031300593 | 735.9142167 | 388.4893927 | 994.5024998 |
| ENSMUSG00000026525 | −1.790734538 | 0.001947167 | 0.0161813 | 7.977389882 | 8.741011335 | 6.427993997 |
| ENSMUSG00000026822 | −1.02651459 | 5.14E−06 | 0.00016724 | 122.6523694 | 119.4604882 | 110.1941828 |
| ENSMUSG00000026826 | −1.483020728 | 0.008821676 | 0.047306802 | 15.95477976 | 10.6834583 | 11.93770314 |
| ENSMUSG00000026832 | −1.174113684 | 0.001522033 | 0.013425591 | 30.91238579 | 34.96404534 | 25.71197599 |
| ENSMUSG00000027360 | −1.979964656 | 1.70E−12 | 3.69E−10 | 54.84455544 | 93.23745424 | 72.54450368 |
| ENSMUSG00000027398 | −1.057691022 | 0.001937845 | 0.016123489 | 78.77672509 | 37.87771578 | 32.13996998 |
| ENSMUSG00000027405 | −1.241634744 | 7.40E−06 | 0.000226395 | 225.3612642 | 254.4605522 | 248.8551962 |
| ENSMUSG00000027496 | −1.007831872 | 0.004782077 | 0.030501955 | 38.88977568 | 37.87771578 | 17.44741228 |
| ENSMUSG00000027513 | −1.115800194 | 1.04E−10 | 1.46E−08 | 16805.36896 | 18645.5484 | 18901.97549 |
| ENSMUSG00000027605 | 1.86697993 | 9.67E−16 | 3.77E−13 | 27327.54622 | 18714.50527 | 21561.32844 |
| ENSMUSG00000027762 | 1.17286147 | 2.98E−15 | 1.13E−12 | 1319.260852 | 1520.935972 | 1404.975831 |
| ENSMUSG00000027907 | −1.140239221 | 0.000303295 | 0.004203282 | 47.86433929 | 56.33096193 | 106.5210434 |
| ENSMUSG00000027947 | −1.05537492 | 2.15E−06 | 8.45E−05 | 434.7677486 | 468.1297181 | 461.8972829 |
| ENSMUSG00000028008 | 1.245889 | 0.000553467 | 0.00652119 | 54.84455544 | 81.58277246 | 55.0970914 |
| ENSMUSG00000028339 | 1.011959422 | 2.14E−08 | 1.71E−06 | 706.9961783 | 505.0362104 | 521.5857986 |
| ENSMUSG00000028445 | −1.767301879 | 0.003925136 | 0.026446185 | 70.79933521 | 71.87053764 | 91.82848567 |
| ENSMUSG00000028630 | 1.331166045 | 1.46E−07 | 8.59E−06 | 373.9401507 | 358.3814647 | 406.8001915 |
| ENSMUSG00000028838 | 1.693729472 | 3.36E−09 | 3.22E−07 | 304.1379893 | 151.5108631 | 198.349529 |
| ENSMUSG00000028859 | −1.001034699 | 0.004539011 | 0.029294129 | 74.78803015 | 57.30218542 | 29.38511541 |
| ENSMUSG00000028862 | −2.278242573 | 1.62E−07 | 9.45E−06 | 8.974563618 | 9.712234816 | 17.44741228 |
| ENSMUSG00000028864 | 1.08474786 | 0.000792949 | 0.008337279 | 131.6269331 | 134.0288405 | 203.8592382 |
| ENSMUSG00000028957 | 2.966674755 | 1.69E−27 | 3.84E−24 | 314.1097266 | 609.9283465 | 341.6019667 |
| ENSMUSG00000028976 | 1.532387058 | 3.09E−07 | 1.58E−05 | 157.5534502 | 94.20867772 | 101.9296191 |
| ENSMUSG00000029086 | 1.631659452 | 1.01E−18 | 6.00E−16 | 713.9763945 | 617.6981343 | 593.2120174 |
| ENSMUSG00000029135 | −1.266157753 | 3.55E−07 | 1.81E−05 | 179.4912724 | 127.2302761 | 136.8244436 |
| ENSMUSG00000029188 | −2.354954255 | 1.89E−12 | 4.02E−10 | 42.87847062 | 12.62590526 | 51.42395197 |
| ENSMUSG00000029195 | 1.272024305 | 6.31E−09 | 5.74E−07 | 705.0018309 | 838.1658646 | 788.8066919 |
| ENSMUSG00000029370 | 1.683633222 | 1.05E−19 | 7.55E−17 | 546.4512069 | 472.9858356 | 579.4377446 |
| ENSMUSG00000029373 | −1.650047987 | 0.002725238 | 0.020577678 | 7.977389882 | 5.82734089 | 9.182848567 |
| ENSMUSG00000029380 | −2.803403219 | 8.38E−26 | 1.43E−22 | 53.84738171 | 128.2014996 | 71.62621882 |
| ENSMUSG00000029580 | −1.162469269 | 9.11E−12 | 1.55E−09 | 8321.414821 | 7155.974613 | 6927.540959 |
| ENSMUSG00000029591 | −1.593385799 | 3.01E−08 | 2.29E−06 | 43.87564435 | 60.21585586 | 35.81310941 |
| ENSMUSG00000029656 | −1.019716092 | 0.000134204 | 0.002299171 | 189.4630097 | 409.8563092 | 189.1666805 |
| ENSMUSG00000030032 | −1.061169015 | 0.007631272 | 0.042668167 | 23.93216965 | 14.56835222 | 25.71197599 |
| ENSMUSG00000030055 | 1.305889925 | 4.54E−06 | 0.000151075 | 239.3216965 | 263.2015635 | 292.9328693 |
| ENSMUSG00000030691 | 1.035229834 | 1.01E−08 | 8.79E−07 | 365.9627609 | 443.8491311 | 415.0647552 |
| ENSMUSG00000030782 | −1.087204324 | 0.00053618 | 0.006369234 | 38.88977568 | 39.82016275 | 40.40453369 |
| ENSMUSG00000030814 | −1.357189589 | 8.78E−10 | 9.81E−08 | 261.2595186 | 283.5972566 | 291.0962996 |
| ENSMUSG00000030827 | −1.631143551 | 1.45E−05 | 0.000383464 | 26.92369085 | 20.39569311 | 33.05825484 |
| ENSMUSG00000030934 | 1.434845811 | 0.000702548 | 0.007652274 | 13671.25191 | 24780.76713 | 9134.17947 |
| ENSMUSG00000030968 | −1.140744433 | 0.000133867 | 0.002296287 | 34.90108074 | 72.84176112 | 54.17880654 |
| ENSMUSG00000031010 | 1.281779593 | 3.26E−07 | 1.66E−05 | 953.2980909 | 1398.561814 | 1420.586673 |
| ENSMUSG00000031271 | −1.719181951 | 2.91E−23 | 3.97E−20 | 339.03907 | 470.0721651 | 364.5590881 |
| ENSMUSG00000031378 | 1.154152634 | 6.32E−15 | 2.21E−12 | 1014.125689 | 912.9500727 | 885.2266019 |
| ENSMUSG00000031465 | −1.254345174 | 0.0029265 | 0.021654196 | 21.93782218 | 11.65468178 | 20.20226685 |
| ENSMUSG00000031762 | −4.701992063 | 1.08E−05 | 0.000305681 | 384.9090618 | 188.4173554 | 32.13996998 |
| ENSMUSG00000031765 | −4.008275951 | 6.98E−08 | 4.64E−06 | 1129.797842 | 691.5111189 | 337.0105424 |
| ENSMUSG00000032009 | 1.096135097 | 4.98E−08 | 3.50E−06 | 816.6852892 | 873.12991 | 1044.089882 |
| ENSMUSG00000032064 | 1.076164714 | 3.75E−10 | 4.69E−08 | 659.131839 | 690.5398954 | 661.1650968 |
| ENSMUSG00000032083 | −1.056710751 | 2.91E−09 | 2.86E−07 | 57416.2665 | 56826.28591 | 68671.17815 |
| ENSMUSG00000032091 | 2.476316256 | 2.69E−06 | 0.000101175 | 102.7088947 | 50.50362104 | 32.13996998 |
| ENSMUSG00000032285 | 1.014640865 | 0.008899206 | 0.047498424 | 51.85303424 | 70.89931416 | 52.34223683 |
| ENSMUSG00000032417 | 1.412452049 | 0.000231229 | 0.003394264 | 102.7088947 | 50.50362104 | 80.80906739 |
| ENSMUSG00000032418 | 1.488424738 | 1.96E−09 | 2.00E−07 | 17569.20404 | 7712.485668 | 13799.06654 |
| ENSMUSG00000032500 | 2.09734091 | 1.65E−20 | 1.25E−17 | 413.8271001 | 367.1224761 | 372.8236518 |
| ENSMUSG00000032561 | 1.808480512 | 0.000942567 | 0.009458265 | 1361.142149 | 660.4319675 | 1176.322901 |
| ENSMUSG00000032702 | 1.072973429 | 8.45E−08 | 5.48E−06 | 1328.235415 | 1308.23803 | 1494.049462 |
| ENSMUSG00000032724 | 1.34458697 | 3.25E−10 | 4.18E−08 | 647.1657542 | 540.0002558 | 680.4490788 |
| ENSMUSG00000032735 | −1.26252931 | 1.21E−08 | 1.04E−06 | 191.4573572 | 257.3742226 | 294.769439 |
| ENSMUSG00000032786 | −1.26794906 | 3.89E−08 | 2.81E−06 | 1778.957944 | 2029.857077 | 2206.638511 |
| ENSMUSG00000032849 | 1.170479352 | 7.00E−10 | 8.09E−08 | 403.8553628 | 338.9569951 | 399.4539127 |
| ENSMUSG00000032860 | 1.046181292 | 6.20E−08 | 4.19E−06 | 339.03907 | 263.2015635 | 290.1780147 |
| ENSMUSG00000032883 | 1.139189345 | 1.88E−11 | 3.09E−09 | 1754.0286 | 1475.288469 | 1528.944286 |
| ENSMUSG00000033105 | 1.210153299 | 1.43E−10 | 1.97E−08 | 3593.814142 | 2741.763889 | 3240.627259 |
| ENSMUSG00000033594 | −1.045179866 | 5.30E−08 | 3.67E−06 | 379.9231931 | 342.841889 | 348.9482455 |
| ENSMUSG00000033624 | 1.545958738 | 3.43E−10 | 4.33E−08 | 290.177557 | 397.230404 | 428.8390281 |
| ENSMUSG00000033792 | 1.183185841 | 0.000561984 | 0.006589659 | 63.81911906 | 67.98564371 | 74.38107339 |
| ENSMUSG00000033855 | 1.291796081 | 0.000477464 | 0.005865923 | 117.6665008 | 69.92809068 | 198.349529 |
| ENSMUSG00000033967 | −2.822222129 | 4.36E−05 | 0.000952114 | 4.985868677 | 3.884893927 | 1.836569713 |
| ENSMUSG00000034066 | 1.2566902 | 2.38E−07 | 1.29E−05 | 275.2199509 | 346.7267829 | 310.3802816 |
| ENSMUSG00000034110 | 1.131917427 | 3.20E−05 | 0.000736514 | 99.71737353 | 110.7194769 | 115.7038919 |
| ENSMUSG00000034271 | −1.155793079 | 0.004733656 | 0.030334993 | 36.89542821 | 29.13670445 | 40.40453369 |
| ENSMUSG00000034755 | −1.51005032 | 0.006510973 | 0.037993212 | 7.977389882 | 5.82734089 | 12.85598799 |
| ENSMUSG00000034765 | −2.457798355 | 3.78E−06 | 0.000131464 | 23.93216965 | 12.62590526 | 9.182848567 |
| ENSMUSG00000034853 | 1.437062013 | 9.64E−14 | 2.63E−11 | 450.7225284 | 335.0721012 | 434.3487372 |
| ENSMUSG00000034926 | 1.093495822 | 1.31E−06 | 5.56E−05 | 17902.26007 | 19066.08817 | 17627.39611 |
| ENSMUSG00000035078 | 1.209517873 | 6.16E−05 | 0.001249206 | 322.0871165 | 384.6044987 | 346.193391 |
| ENSMUSG00000035112 | −1.146398835 | 0.004042609 | 0.02701081 | 23.93216965 | 14.56835222 | 18.36569713 |
| ENSMUSG00000035164 | 1.06213892 | 8.60E−06 | 0.000256206 | 131.6269331 | 166.0792154 | 141.4158679 |
| ENSMUSG00000035165 | −1.798528938 | 1.18E−06 | 5.05E−05 | 32.90673326 | 22.33814008 | 28.46683056 |
| ENSMUSG00000035284 | 1.432291641 | 4.22E−09 | 3.95E−07 | 725.9424793 | 940.1443302 | 1017.459621 |
| ENSMUSG00000035900 | 1.041020638 | 2.29E−08 | 1.81E−06 | 343.0277649 | 318.561302 | 367.3139427 |
| ENSMUSG00000035933 | 1.189062904 | 7.63E−07 | 3.47E−05 | 188.465836 | 221.4389538 | 244.2637719 |
| ENSMUSG00000035948 | 1.741049509 | 1.78E−10 | 2.38E−08 | 816.6852892 | 893.5256031 | 857.6780562 |
| ENSMUSG00000036062 | −1.708320866 | 0.004715534 | 0.030261525 | 7.977389882 | 9.712234816 | 3.673139427 |
| ENSMUSG00000036120 | −1.340248384 | 3.85E−09 | 3.64E−07 | 263.2538661 | 219.4965068 | 224.9797899 |
| ENSMUSG00000036611 | 1.04665245 | 4.08E−07 | 2.03E−05 | 846.6005013 | 616.7269108 | 585.8657386 |
| ENSMUSG00000037035 | −1.785829601 | 2.14E−10 | 2.84E−08 | 59.83042412 | 30.10792793 | 50.50566712 |
| ENSMUSG00000037071 | 1.600161226 | 3.89E−11 | 5.71E−09 | 372268.8876 | 280319.3774 | 340820.5063 |
| ENSMUSG00000037095 | −1.027049761 | 7.90E−10 | 8.97E−08 | 3492.102421 | 3177.843232 | 2991.772063 |
| ENSMUSG00000037157 | 1.742314913 | 8.42E−06 | 0.000251859 | 51.85303424 | 60.21585586 | 58.77023083 |
| ENSMUSG00000037336 | −1.65964734 | 0.000936801 | 0.009424872 | 14.95760603 | 7.769787853 | 8.26456371 |
| ENSMUSG00000037443 | −1.349128808 | 2.89E−12 | 5.72E−10 | 750.8718227 | 1078.058065 | 1089.08584 |
| ENSMUSG00000037447 | −1.380120078 | 0.000135203 | 0.002310483 | 30.91238579 | 23.30936356 | 25.71197599 |
| ENSMUSG00000037465 | −1.250556349 | 2.44E−12 | 4.99E−10 | 342.0305912 | 272.9137983 | 297.5242936 |
| ENSMUSG00000037583 | −1.178511493 | 2.71E−06 | 0.000101645 | 181.4856198 | 270.0001279 | 213.9603716 |
| ENSMUSG00000037709 | 1.621036788 | 2.49E−19 | 1.62E−16 | 850.5891962 | 1019.784656 | 857.6780562 |
| ENSMUSG00000037887 | −1.887921138 | 0.000434734 | 0.005464027 | 15.95477976 | 8.741011335 | 11.01941828 |
| ENSMUSG00000038217 | 1.023783748 | 9.74E−10 | 1.07E−07 | 3279.704415 | 3214.749724 | 3049.624009 |
| ENSMUSG00000038233 | 1.132248981 | 2.75E−05 | 0.000651686 | 765.8294287 | 340.8994421 | 856.7597713 |
| ENSMUSG00000038253 | −2.17957789 | 5.49E−08 | 3.76E−06 | 19.94347471 | 18.45324615 | 14.69255771 |
| ENSMUSG00000038370 | −1.863884427 | 6.22E−10 | 7.32E−08 | 95.72867859 | 197.1583668 | 141.4158679 |
| ENSMUSG00000038415 | −1.598903332 | 5.74E−10 | 6.80E−08 | 144.5901916 | 136.9425109 | 205.6958079 |
| ENSMUSG00000038418 | −2.528561862 | 1.50E−17 | 7.58E−15 | 95.72867859 | 79.64032549 | 131.3147345 |
| ENSMUSG00000038473 | 1.076370271 | 0.000625033 | 0.007061263 | 117.6665008 | 63.12952631 | 89.99191596 |
| ENSMUSG00000038530 | −1.722746304 | 4.58E−05 | 0.00098969 | 17.94912724 | 16.51079919 | 13.77427285 |
| ENSMUSG00000038583 | −1.702074711 | 0.00088397 | 0.009002758 | 12.96325856 | 13.59712874 | 11.93770314 |
| ENSMUSG00000038587 | −1.31455808 | 3.12E−06 | 0.000113043 | 97.72302606 | 73.8129846 | 141.4158679 |
| ENSMUSG00000038751 | 1.774904606 | 6.46E−12 | 1.16E−09 | 167.5251875 | 125.2878291 | 171.7192682 |
| ENSMUSG00000038768 | 1.297337324 | 1.64E−05 | 0.000422782 | 329.0673326 | 229.2087417 | 124.8867405 |
| ENSMUSG00000038774 | 1.007283954 | 3.14E−06 | 0.00011311 | 450.7225284 | 497.2664226 | 488.5275438 |
| ENSMUSG00000038844 | 1.003328537 | 1.78E−06 | 7.13E−05 | 364.9655871 | 380.7196048 | 386.5979247 |
| ENSMUSG00000038895 | −1.113485639 | 6.66E−08 | 4.45E−06 | 100.7145473 | 103.9209125 | 94.58334024 |
| ENSMUSG00000039103 | −1.109845105 | 0.002277587 | 0.018174053 | 37.89260194 | 21.3669166 | 26.63026084 |
| ENSMUSG00000039304 | 1.09876453 | 1.10E−06 | 4.76E−05 | 197.4403996 | 176.7626737 | 181.8204016 |
| ENSMUSG00000039533 | 1.597388363 | 4.59E−08 | 3.26E−06 | 367.9571083 | 378.7771578 | 293.8511541 |
| ENSMUSG00000039601 | 1.185885146 | 6.77E−14 | 1.97E−11 | 707.9933521 | 644.8923918 | 732.7913156 |
| ENSMUSG00000039704 | 1.942291849 | 0.00175377 | 0.014994461 | 267.2425611 | 391.4030631 | 463.7338526 |
| ENSMUSG00000039741 | 1.087450189 | 5.12E−08 | 3.58E−06 | 264.2510399 | 270.0001279 | 292.0145844 |
| ENSMUSG00000039853 | 1.374596414 | 3.52E−09 | 3.36E−07 | 477.6462192 | 438.0217902 | 491.2823983 |
| ENSMUSG00000039981 | 1.024216498 | 5.34E−05 | 0.001113305 | 116.669327 | 128.2014996 | 147.8438619 |
| ENSMUSG00000040093 | 1.487860932 | 2.33E−07 | 1.27E−05 | 234.3358278 | 188.4173554 | 357.2128093 |
| ENSMUSG00000040128 | −1.050165381 | 1.95E−11 | 3.16E−09 | 1378.094102 | 1310.180477 | 1381.100424 |
| ENSMUSG00000040152 | −1.254644006 | 0.001007039 | 0.009915514 | 35.89825447 | 19.42446963 | 35.81310941 |
| ENSMUSG00000040435 | −1.205190782 | 4.71E−06 | 0.000155637 | 119.6608482 | 131.11517 | 127.6415951 |
| ENSMUSG00000040584 | 1.185136822 | 2.65E−06 | 9.97E−05 | 302.1436418 | 375.8634874 | 406.8001915 |
| ENSMUSG00000040855 | 1.071792937 | 4.60E−07 | 2.25E−05 | 584.3438089 | 647.8060622 | 645.5542543 |
| ENSMUSG00000040891 | −2.088765162 | 3.05E−41 | 2.08E−37 | 406.846884 | 431.2232258 | 360.8859487 |
| ENSMUSG00000041134 | 1.050155799 | 0.006573499 | 0.03826156 | 41.88129688 | 57.30218542 | 84.48220682 |
| ENSMUSG00000041372 | 2.023614953 | 1.33E−05 | 0.000359411 | 51.85303424 | 49.53239756 | 28.46683056 |
| ENSMUSG00000041695 | −1.532466883 | 9.09E−05 | 0.001683702 | 33.903907 | 18.45324615 | 23.87540627 |
| ENSMUSG00000041702 | 1.159708962 | 1.79E−05 | 0.000451095 | 125.6438906 | 178.7051206 | 179.0655471 |
| ENSMUSG00000041920 | −1.612549242 | 5.78E−11 | 8.29E−09 | 130.6297593 | 116.5468178 | 162.5364196 |
| ENSMUSG00000041930 | −2.069670842 | 7.78E−10 | 8.92E−08 | 29.91521206 | 25.25181052 | 29.38511541 |
| ENSMUSG00000041945 | 2.034436558 | 0.000645414 | 0.007184911 | 35.89825447 | 23.30936356 | 20.20226685 |
| ENSMUSG00000042010 | 1.335313237 | 1.72E−08 | 1.42E−06 | 9652.641758 | 5265.973717 | 7079.05796 |
| ENSMUSG00000042115 | −1.249312452 | 6.34E−07 | 3.01E−05 | 57.83607665 | 47.5899506 | 51.42395197 |
| ENSMUSG00000042246 | −1.458803387 | 0.000333225 | 0.004503661 | 16.9519535 | 19.42446963 | 23.87540627 |
| ENSMUSG00000042333 | 1.17659167 | 0.000212792 | 0.003210009 | 83.76259377 | 76.72665505 | 64.27993997 |
| ENSMUSG00000042354 | −1.004338102 | 0.000195353 | 0.003017019 | 312.1153791 | 284.5684801 | 269.057463 |
| ENSMUSG00000042379 | −1.128744357 | 0.001182685 | 0.011115132 | 63.81911906 | 39.82016275 | 89.99191596 |
| ENSMUSG00000042444 | 1.165704626 | 4.84E−10 | 5.85E−08 | 592.3211988 | 520.5757862 | 674.0210848 |
| ENSMUSG00000042510 | −1.154445577 | 0.002024484 | 0.016651324 | 28.91803832 | 29.13670445 | 26.63026084 |
| ENSMUSG00000042607 | −1.584091426 | 0.001074406 | 0.010435667 | 12.96325856 | 6.798564371 | 14.69255771 |
| ENSMUSG00000042622 | −1.343729967 | 0.000106867 | 0.001912519 | 29.91521206 | 22.33814008 | 31.22168513 |
| ENSMUSG00000042680 | 1.086900564 | 0.000262276 | 0.00374505 | 262.2566924 | 328.2735368 | 358.1310941 |
| ENSMUSG00000042743 | 1.143476031 | 0.005459928 | 0.033421057 | 39.88694941 | 35.93526882 | 50.50566712 |
| ENSMUSG00000042745 | −1.243661764 | 2.94E−08 | 2.25E−06 | 425.793185 | 215.6116129 | 317.7265604 |
| ENSMUSG00000043165 | −1.65740279 | 0.002445867 | 0.019147761 | 6.980216147 | 13.59712874 | 9.182848567 |
| ENSMUSG00000043421 | −1.515224307 | 3.07E−05 | 0.000711553 | 31.90955953 | 28.16548097 | 33.9765397 |
| ENSMUSG00000043639 | 1.042566076 | 0.00209562 | 0.017092091 | 83.76259377 | 70.89931416 | 87.23706139 |
| ENSMUSG00000043681 | −1.124534949 | 2.43E−09 | 2.42E−07 | 201.4290945 | 238.9209765 | 202.0226685 |
| ENSMUSG00000044042 | 1.154056163 | 3.06E−05 | 0.000711225 | 258.2679974 | 274.8562453 | 353.5396698 |
| ENSMUSG00000044186 | 1.148916053 | 0.009243418 | 0.048746497 | 35.89825447 | 33.99282186 | 30.30340027 |
| ENSMUSG00000044339 | −1.202694639 | 0.000240284 | 0.0035008 | 44.87281809 | 27.19425749 | 28.46683056 |
| ENSMUSG00000044349 | 1.020247724 | 0.000864482 | 0.008883909 | 170.5167087 | 218.5252834 | 182.7386865 |
| ENSMUSG00000044359 | 1.419594491 | 2.01E−07 | 1.12E−05 | 221.3725692 | 303.9929497 | 263.5477539 |
| ENSMUSG00000044676 | −1.235532178 | 3.06E−06 | 0.000111153 | 173.5082299 | 206.8706016 | 146.0072922 |
| ENSMUSG00000044749 | 1.260739468 | 0.003720434 | 0.025546608 | 2581.682801 | 3424.533996 | 3631.816608 |
| ENSMUSG00000044948 | −1.380324526 | 9.28E−05 | 0.001711935 | 28.91803832 | 18.45324615 | 23.87540627 |
| ENSMUSG00000045045 | 1.563883127 | 9.68E−08 | 6.14E−06 | 106.6975897 | 117.5180413 | 171.7192682 |
| ENSMUSG00000045294 | 1.216661032 | 2.49E−17 | 1.21E−14 | 16820.32657 | 15644.46784 | 18334.47545 |
| ENSMUSG00000045348 | −1.535176629 | 0.002140436 | 0.017319005 | 17.94912724 | 8.741011335 | 9.182848567 |
| ENSMUSG00000045382 | −1.705364762 | 8.30E−05 | 0.001575106 | 17.94912724 | 15.53957571 | 23.87540627 |
| ENSMUSG00000045411 | −1.265704961 | 6.98E−06 | 0.000215157 | 217.3838743 | 180.6475676 | 227.7346445 |
| ENSMUSG00000045776 | 1.435732199 | 4.30E−15 | 1.59E−12 | 1029.083295 | 1166.439401 | 1214.890865 |
| ENSMUSG00000045875 | 1.205673082 | 0.000172777 | 0.002749319 | 91.73998365 | 131.11517 | 117.5404617 |
| ENSMUSG00000046541 | 1.523461987 | 5.84E−06 | 0.000185032 | 69.80216147 | 95.1799012 | 84.48220682 |
| ENSMUSG00000046721 | −1.016110295 | 0.006334733 | 0.037187581 | 17.94912724 | 26.223034 | 22.95712142 |
| ENSMUSG00000046908 | −1.354651164 | 0.005396039 | 0.033236578 | 15.95477976 | 8.741011335 | 10.10113342 |
| ENSMUSG00000047496 | 1.093888518 | 1.32E−05 | 0.00035939 | 433.7705749 | 520.5757862 | 451.7961495 |
| ENSMUSG00000047649 | −1.227032883 | 5.45E−06 | 0.000175137 | 72.79368268 | 67.01442023 | 64.27993997 |
| ENSMUSG00000047875 | 1.019701978 | 0.000156296 | 0.002567963 | 101.711721 | 104.892136 | 85.40049167 |
| ENSMUSG00000048191 | −1.584188448 | 0.000509157 | 0.006122909 | 10.96891109 | 18.45324615 | 11.01941828 |
| ENSMUSG00000048644 | −2.138371107 | 3.35E−05 | 0.00076585 | 5.983042412 | 8.741011335 | 8.26456371 |
| ENSMUSG00000048856 | −1.529839383 | 8.95E−23 | 8.72E−20 | 3855.073661 | 3458.526818 | 4005.558545 |
| ENSMUSG00000049044 | −1.523296713 | 7.27E−17 | 3.42E−14 | 783.7785559 | 1199.461 | 1266.314817 |
| ENSMUSG00000049313 | 1.020089742 | 3.99E−06 | 0.000136353 | 328.0701589 | 236.9785295 | 312.2168513 |
| ENSMUSG00000049580 | −1.860685334 | 2.05E−30 | 5.59E−27 | 548.4455544 | 407.9138623 | 479.3446952 |
| ENSMUSG00000049791 | 1.116973741 | 1.01E−06 | 4.43E−05 | 636.1968431 | 678.8852137 | 759.4215765 |
| ENSMUSG00000049950 | −1.164244145 | 0.007174451 | 0.040867998 | 18.94630097 | 37.87771578 | 21.1205517 |
| ENSMUSG00000050390 | 1.148547066 | 4.80E−15 | 1.72E−12 | 1574.537328 | 1571.439593 | 1292.026793 |
| ENSMUSG00000050503 | −1.747508686 | 0.001612805 | 0.014098606 | 6.980216147 | 9.712234816 | 7.346278854 |
| ENSMUSG00000050663 | 1.366962359 | 0.003061462 | 0.022321797 | 20.94064844 | 51.47484453 | 44.07767312 |
| ENSMUSG00000050737 | −1.262831751 | 0.008424555 | 0.045643887 | 26.92369085 | 13.59712874 | 44.99595798 |
| ENSMUSG00000050914 | −2.867341906 | 0.000724516 | 0.007829023 | 13.96043229 | 23.30936356 | 14.69255771 |
| ENSMUSG00000051149 | 1.736018564 | 0.001879809 | 0.015833824 | 31.90955953 | 33.02159838 | 39.48624884 |
| ENSMUSG00000051339 | 1.051356464 | 2.17E−16 | 9.53E−14 | 1923.548135 | 1976.439785 | 1850.343986 |
| ENSMUSG00000051452 | 1.412708477 | 1.55E−10 | 2.11E−08 | 276.2171247 | 240.8634234 | 283.7500207 |
| ENSMUSG00000051674 | 1.054513908 | 9.53E−09 | 8.39E−07 | 515.5388212 | 743.9571869 | 608.82286 |
| ENSMUSG00000051998 | −1.5364341 | 0.002646663 | 0.020230437 | 9.971737353 | 19.42446963 | 6.427993997 |
| ENSMUSG00000052085 | 1.272820714 | 3.30E−11 | 4.99E−09 | 423.7988375 | 546.7988202 | 446.2864404 |
| ENSMUSG00000052595 | 1.233431784 | 0.003777269 | 0.025781088 | 1666.277312 | 2467.878867 | 2847.601341 |
| ENSMUSG00000052656 | 1.063492715 | 4.09E−12 | 7.63E−10 | 4427.451385 | 3448.814583 | 3382.043127 |
| ENSMUSG00000052684 | −1.26752533 | 1.09E−07 | 6.78E−06 | 161.5421451 | 161.223098 | 181.8204016 |
| ENSMUSG00000052713 | 1.631079765 | 2.29E−06 | 8.94E−05 | 74.78803015 | 84.4964429 | 102.8479039 |
| ENSMUSG00000052837 | −1.91589408 | 1.98E−18 | 1.08E−15 | 205.4177895 | 302.0505028 | 276.4037419 |
| ENSMUSG00000053560 | −1.145431158 | 4.41E−10 | 5.44E−08 | 324.081464 | 268.0576809 | 242.4272022 |
| ENSMUSG00000053964 | −1.860738134 | 2.78E−14 | 8.62E−12 | 100.7145473 | 119.4604882 | 153.3535711 |
| ENSMUSG00000053977 | −1.428502316 | 0.007562752 | 0.042337131 | 8.974563618 | 28.16548097 | 7.346278854 |
| ENSMUSG00000054008 | 1.359856014 | 0.005424595 | 0.033352211 | 3190.955953 | 3047.699285 | 3392.144261 |
| ENSMUSG00000054150 | 1.583267643 | 1.23E−07 | 7.44E−06 | 98.72019979 | 105.8633595 | 78.97249768 |
| ENSMUSG00000054422 | −1.006699851 | 5.17E−07 | 2.51E−05 | 21378.40771 | 26089.97639 | 27702.81756 |
| ENSMUSG00000054453 | 1.090734081 | 0.000462815 | 0.005711685 | 93.73433112 | 87.41011335 | 126.7233102 |
| ENSMUSG00000054659 | 1.244126007 | 0.002259781 | 0.018053098 | 79.77389882 | 73.8129846 | 100.0930494 |
| ENSMUSG00000054932 | 1.219377704 | 9.42E−05 | 0.001724207 | 87.75128871 | 202.0144842 | 233.2443536 |
| ENSMUSG00000055148 | −1.210391107 | 1.04E−07 | 6.56E−06 | 210.4036581 | 153.4533101 | 137.7427285 |
| ENSMUSG00000055254 | 1.702350158 | 1.36E−13 | 3.56E−11 | 1260.427601 | 1100.396205 | 2139.603716 |
| ENSMUSG00000055491 | −1.429157403 | 5.36E−08 | 3.69E−06 | 191.4573572 | 205.8993781 | 144.1707225 |
| ENSMUSG00000055660 | 1.21539125 | 9.47E−05 | 0.001725945 | 86.75411497 | 97.12234816 | 112.0307525 |
| ENSMUSG00000055692 | −1.507668302 | 6.91E−05 | 0.001367589 | 23.93216965 | 24.28058704 | 19.28398199 |
| ENSMUSG00000055980 | 1.007478258 | 8.74E−05 | 0.001638705 | 336.0475488 | 377.8059344 | 381.0882155 |
| ENSMUSG00000056054 | −4.00774487 | 1.39E−20 | 1.11E−17 | 6.980216147 | 6.798564371 | 4.591424283 |
| ENSMUSG00000056071 | −3.639131514 | 1.15E−24 | 1.74E−21 | 15.95477976 | 8.741011335 | 12.85598799 |
| ENSMUSG00000056091 | −1.031452251 | 1.62E−09 | 1.68E−07 | 1990.358776 | 1481.115809 | 2190.109383 |
| ENSMUSG00000056148 | 1.3596028 | 0.000119043 | 0.002094688 | 75.78520388 | 108.7770299 | 71.62621882 |
| ENSMUSG00000056313 | −1.726808914 | 2.85E−11 | 4.37E−09 | 435.7649223 | 244.7483174 | 415.0647552 |
| ENSMUSG00000057342 | 1.09304789 | 8.77E−15 | 2.92E−12 | 1269.402165 | 1384.964685 | 1236.011417 |
| ENSMUSG00000057604 | −1.272122676 | 0.008168023 | 0.044709526 | 10.96891109 | 10.6834583 | 11.93770314 |
| ENSMUSG00000057722 | −1.840565518 | 1.78E−07 | 1.02E−05 | 45.86999182 | 35.93526882 | 110.1941828 |
| ENSMUSG00000057969 | 1.066228289 | 0.000219235 | 0.003267437 | 95.72867859 | 74.78420809 | 99.17476452 |
| ENSMUSG00000058207 | −1.338541988 | 1.08E−13 | 2.88E−11 | 23432.58561 | 23413.28447 | 28330.9244 |
| ENSMUSG00000058503 | −1.136818503 | 6.67E−07 | 3.10E−05 | 130.6297593 | 164.1367684 | 153.3535711 |
| ENSMUSG00000058793 | 1.056167061 | 4.57E−10 | 5.57E−08 | 3494.096768 | 3071.008649 | 3164.409616 |
| ENSMUSG00000058794 | 1.61977926 | 2.23E−06 | 8.73E−05 | 144.5901916 | 102.9496891 | 144.1707225 |
| ENSMUSG00000058921 | 1.372756574 | 4.51E−07 | 2.22E−05 | 911.4167941 | 1126.619239 | 1263.559963 |
| ENSMUSG00000059149 | 1.084964814 | 0.00015449 | 0.002547498 | 343.0277649 | 296.2231619 | 348.9482455 |
| ENSMUSG00000059824 | 4.510710868 | 5.51E−13 | 1.32E−10 | 248.2962601 | 730.3600582 | 248.8551962 |
| ENSMUSG00000060429 | 1.717674566 | 1.22E−12 | 2.68E−10 | 734.9170429 | 831.3673003 | 944.9151175 |
| ENSMUSG00000061175 | 1.152852085 | 3.29E−14 | 9.98E−12 | 1100.879804 | 976.079599 | 989.9110755 |
| ENSMUSG00000061292 | 1.112980568 | 1.04E−05 | 0.000296719 | 1260.427601 | 1520.935972 | 1350.797024 |
| ENSMUSG00000061436 | 1.042732241 | 2.40E−05 | 0.000578522 | 326.0758114 | 431.2232258 | 663.9199514 |
| ENSMUSG00000061536 | 1.045389319 | 1.00E−05 | 0.000287463 | 219.3782218 | 260.2878931 | 199.2678139 |
| ENSMUSG00000061825 | 1.826944112 | 9.41E−14 | 2.63E−11 | 718.9622632 | 690.5398954 | 678.6125091 |
| ENSMUSG00000062901 | 1.319446488 | 6.22E−09 | 5.70E−07 | 788.7644246 | 893.5256031 | 992.6659301 |
| ENSMUSG00000063535 | 1.201850675 | 0.000151738 | 0.002508185 | 62.82194532 | 71.87053764 | 70.70793397 |
| ENSMUSG00000063704 | −1.127471271 | 2.47E−07 | 1.32E−05 | 294.1662519 | 311.7627376 | 224.061505 |
| ENSMUSG00000063929 | −1.198836957 | 5.83E−06 | 0.000185032 | 50.8558605 | 105.8633595 | 82.6456371 |
| ENSMUSG00000065126 | −1.713055356 | 1.38E−07 | 8.18E−06 | 29.91521206 | 48.56117408 | 40.40453369 |
| ENSMUSG00000065147 | −1.294541943 | 0.007255788 | 0.041125179 | 14.95760603 | 12.62590526 | 15.61084256 |
| ENSMUSG00000065952 | 1.57267976 | 1.62E−10 | 2.19E−08 | 199.4347471 | 157.338204 | 188.2483956 |
| ENSMUSG00000066456 | 1.032022635 | 0.005405213 | 0.033278057 | 76.78237762 | 64.10074979 | 150.5987165 |
| ENSMUSG00000066477 | −1.128543552 | 2.29E−05 | 0.000561201 | 57.83607665 | 44.67628015 | 49.58738226 |
| ENSMUSG00000066687 | −2.591322343 | 5.98E−23 | 6.28E−20 | 139.6043229 | 122.3741587 | 161.6181348 |
| ENSMUSG00000066944 | 1.544707207 | 0.000412457 | 0.005261624 | 43.87564435 | 49.53239756 | 40.40453369 |
| ENSMUSG00000067149 | −1.843573184 | 0.000282252 | 0.003980428 | 113.6778058 | 169.9641093 | 162.5364196 |
| ENSMUSG00000068463 | −2.382320454 | 0.000472976 | 0.005821273 | 4.985868677 | 3.884893927 | 5.50970914 |
| ENSMUSG00000068742 | 1.190690012 | 3.19E−12 | 6.14E−10 | 611.2674997 | 770.1802209 | 691.4684971 |
| ENSMUSG00000068877 | −1.095647878 | 6.50E−07 | 3.04E−05 | 98.72019979 | 98.09357164 | 136.8244436 |
| ENSMUSG00000069456 | 1.727916701 | 1.48E−26 | 2.89E−23 | 6191.451722 | 5969.139518 | 5731.934075 |
| ENSMUSG00000069804 | −2.067148134 | 1.72E−05 | 0.000437213 | 3.988694941 | 13.59712874 | 13.77427285 |
| ENSMUSG00000070576 | 1.095801322 | 1.06E−07 | 6.63E−06 | 253.2821288 | 274.8562453 | 226.8163596 |
| ENSMUSG00000070583 | 1.188428844 | 0.006226465 | 0.036681868 | 41.88129688 | 47.5899506 | 40.40453369 |
| ENSMUSG00000071076 | −1.243260003 | 3.56E−08 | 2.62E−06 | 1324.24672 | 1340.288405 | 938.4871235 |
| ENSMUSG00000071456 | 1.345823726 | 0.001263422 | 0.011712633 | 45.86999182 | 70.89931416 | 48.6690974 |
| ENSMUSG00000071547 | −1.168738292 | 0.00025901 | 0.003706323 | 44.87281809 | 25.25181052 | 47.75081255 |
| ENSMUSG00000071637 | −1.028910519 | 0.000250761 | 0.0036106 | 107.6947634 | 64.10074979 | 89.99191596 |
| ENSMUSG00000071645 | −1.14015686 | 5.54E−07 | 2.68E−05 | 206.4149632 | 273.8850218 | 188.2483956 |
| ENSMUSG00000072294 | 2.084673623 | 3.53E−08 | 2.62E−06 | 97.72302606 | 148.5971927 | 196.5129593 |
| ENSMUSG00000072571 | 1.01455077 | 0.003907572 | 0.026379983 | 56.83890291 | 47.5899506 | 55.0970914 |
| ENSMUSG00000072664 | 1.275253331 | 1.57E−06 | 6.41E−05 | 951.3037435 | 1401.475484 | 1131.326943 |
| ENSMUSG00000072692 | −1.135811828 | 0.000365333 | 0.004818223 | 34.90108074 | 29.13670445 | 28.46683056 |
| ENSMUSG00000072849 | −2.106422019 | 7.14E−21 | 6.08E−18 | 100.7145473 | 96.15112468 | 102.8479039 |
| ENSMUSG00000072999 | −1.145851135 | 1.86E−07 | 1.05E−05 | 116.669327 | 176.7626737 | 168.0461288 |
| ENSMUSG00000073460 | −1.28577089 | 0.005211876 | 0.032439227 | 18.94630097 | 39.82016275 | 10.10113342 |
| ENSMUSG00000073835 | 2.295482419 | 0.000631649 | 0.007089542 | 332.0588539 | 185.503685 | 325.0728393 |
| ENSMUSG00000074024 | −1.08003527 | 4.28E−05 | 0.000939631 | 43.87564435 | 61.18707934 | 53.26052169 |
| ENSMUSG00000074063 | −2.210318446 | 1.23E−07 | 7.44E−06 | 989.1963454 | 819.7126185 | 1493.131177 |
| ENSMUSG00000074213 | 1.225081814 | 0.003513884 | 0.024460869 | 87.75128871 | 38.84893927 | 37.64967912 |
| ENSMUSG00000074345 | 1.610694413 | 1.86E−06 | 7.43E−05 | 92.73715738 | 95.1799012 | 56.93366111 |
| ENSMUSG00000074375 | 1.123374183 | 1.71E−16 | 7.76E−14 | 1739.070994 | 2059.965005 | 1850.343986 |
| ENSMUSG00000074876 | 1.013446872 | 0.000205306 | 0.003120937 | 101.711721 | 119.4604882 | 117.5404617 |
| ENSMUSG00000075470 | 1.495450545 | 0.008033121 | 0.044154642 | 346.0192861 | 387.5181692 | 422.4110341 |
| ENSMUSG00000075552 | 1.02486804 | 2.18E−07 | 1.20E−05 | 1632.373405 | 1558.813688 | 2640.068963 |
| ENSMUSG00000075590 | −1.36961476 | 1.11E−14 | 3.51E−12 | 556.4229443 | 571.0794072 | 507.8115258 |
| ENSMUSG00000076490 | −1.690670704 | 0.002848332 | 0.021272015 | 9.971737353 | 4.856117408 | 8.26456371 |
| ENSMUSG00000076569 | −1.340585852 | 0.000314538 | 0.004311858 | 26.92369085 | 70.89931416 | 46.83252769 |
| ENSMUSG00000076596 | −3.365323343 | 1.13E−07 | 6.96E−06 | 3.988694941 | 6.798564371 | 9.182848567 |
| ENSMUSG00000076609 | −2.421594655 | 3.22E−06 | 0.000115075 | 484.6264354 | 675.0003197 | 704.3244851 |
| ENSMUSG00000076613 | −1.082440472 | 0.000721125 | 0.007817157 | 31.90955953 | 61.18707934 | 74.38107339 |
| ENSMUSG00000076617 | −1.218011643 | 6.44E−08 | 4.32E−06 | 1755.025774 | 1885.144778 | 1859.526835 |
| ENSMUSG00000076934 | −3.037010136 | 1.44E−09 | 1.52E−07 | 7.977389882 | 2.913670445 | 10.10113342 |
| ENSMUSG00000077148 | 1.938039708 | 8.47E−06 | 0.000252598 | 48.86151303 | 38.84893927 | 69.78964911 |
| ENSMUSG00000078193 | −1.269506646 | 0.004582659 | 0.029506003 | 13.96043229 | 9.712234816 | 21.1205517 |
| ENSMUSG00000078234 | 1.031251449 | 6.47E−07 | 3.04E−05 | 557.420118 | 503.0937635 | 599.6400114 |
| ENSMUSG00000078650 | −1.017019231 | 3.99E−07 | 2.00E−05 | 5816.514398 | 3628.490927 | 4180.950953 |
| ENSMUSG00000078651 | −2.099102297 | 6.20E−07 | 2.95E−05 | 8.974563618 | 10.6834583 | 16.52912742 |
| ENSMUSG00000078672 | −1.051061583 | 1.66E−05 | 0.000428362 | 108.6919371 | 197.1583668 | 152.4352862 |
| ENSMUSG00000078688 | 1.787709931 | 3.76E−11 | 5.58E−09 | 119.6608482 | 143.7410753 | 173.5558379 |
| ENSMUSG00000078817 | −1.628755428 | 1.37E−11 | 2.31E−09 | 107.6947634 | 79.64032549 | 80.80906739 |
| ENSMUSG00000079017 | −1.001509959 | 0.000541038 | 0.006404635 | 74.78803015 | 60.21585586 | 44.07767312 |
| ENSMUSG00000079036 | −1.378588057 | 0.008915895 | 0.047568881 | 170.5167087 | 124.3166056 | 106.5210434 |
| ENSMUSG00000079065 | −1.153356662 | 4.39E−06 | 0.000146637 | 123.6495432 | 164.1367684 | 125.8050254 |
| ENSMUSG00000079465 | −1.030415111 | 0.006780187 | 0.03914539 | 18.94630097 | 27.19425749 | 22.03883656 |
| ENSMUSG00000079470 | 1.222302248 | 2.09E−11 | 3.33E−09 | 498.5868677 | 463.2736007 | 474.7532709 |
| ENSMUSG00000080059 | −1.546208951 | 0.008392591 | 0.045561211 | 7.977389882 | 9.712234816 | 6.427993997 |
| ENSMUSG00000081344 | −1.007584184 | 0.007335419 | 0.041406148 | 22.93499591 | 19.42446963 | 38.56796398 |
| ENSMUSG00000082065 | 2.441656125 | 0.003600075 | 0.02496215 | 179.4912724 | 141.7986283 | 255.2831902 |
| ENSMUSG00000082173 | 2.591199516 | 2.10E−11 | 3.33E−09 | 150.573234 | 114.6043708 | 224.061505 |
| ENSMUSG00000082586 | 1.935822229 | 3.52E−06 | 0.000123572 | 53.84738171 | 55.35973845 | 47.75081255 |
| ENSMUSG00000082658 | −1.204694651 | 0.004700851 | 0.030195718 | 67.807814 | 23.30936356 | 23.87540627 |
| ENSMUSG00000083327 | 1.031096879 | 0.000937166 | 0.009424872 | 102.7088947 | 96.15112468 | 82.6456371 |
| ENSMUSG00000083621 | −1.163358375 | 0.003315291 | 0.023547198 | 19.94347471 | 19.42446963 | 20.20226685 |
| ENSMUSG00000083716 | −1.168157812 | 0.001666635 | 0.01443041 | 25.92651712 | 25.25181052 | 35.81310941 |
| ENSMUSG00000083813 | −1.588176086 | 8.35E−05 | 0.001580916 | 11.96608482 | 53.41729149 | 22.03883656 |
| ENSMUSG00000083863 | −1.215047324 | 0.000627696 | 0.007061263 | 24.92934338 | 19.42446963 | 27.5485457 |
| ENSMUSG00000083992 | −1.283026783 | 0.003457083 | 0.024176532 | 11.96608482 | 12.62590526 | 20.20226685 |
| ENSMUSG00000084822 | −1.954137616 | 1.52E−07 | 8.88E−06 | 16.9519535 | 25.25181052 | 13.77427285 |
| ENSMUSG00000084883 | 1.839966406 | 9.14E−09 | 8.09E−07 | 157.5534502 | 141.7986283 | 126.7233102 |
| ENSMUSG00000085001 | −2.14213554 | 3.42E−08 | 2.56E−06 | 29.91521206 | 16.51079919 | 21.1205517 |
| ENSMUSG00000085156 | −1.793249778 | 0.00014396 | 0.002408808 | 24.92934338 | 31.07915141 | 16.52912742 |
| ENSMUSG00000085445 | −1.469856228 | 8.55E−08 | 5.52E−06 | 45.86999182 | 46.61872712 | 72.54450368 |
| ENSMUSG00000085834 | −3.692490083 | 4.07E−39 | 1.85E−35 | 50.8558605 | 56.33096193 | 71.62621882 |
| ENSMUSG00000085995 | −1.230019641 | 9.11E−06 | 0.000268289 | 189.4630097 | 167.0504388 | 353.5396698 |
| ENSMUSG00000086140 | 1.216209638 | 0.003436989 | 0.024085413 | 34.90108074 | 60.21585586 | 44.99595798 |
| ENSMUSG00000086446 | −2.158985376 | 0.000113634 | 0.002012508 | 17.94912724 | 6.798564371 | 9.182848567 |
| ENSMUSG00000086529 | 2.155075918 | 4.95E−05 | 0.001053778 | 27.92086459 | 38.84893927 | 44.07767312 |
| ENSMUSG00000086786 | 1.909493382 | 0.004138057 | 0.027366965 | 31.90955953 | 14.56835222 | 33.05825484 |
| ENSMUSG00000086844 | −1.797314635 | 1.20E−05 | 0.000331862 | 10.96891109 | 17.48202267 | 18.36569713 |
| ENSMUSG00000087382 | −1.074896574 | 1.10E−05 | 0.000308734 | 161.5421451 | 285.5397036 | 136.8244436 |
| ENSMUSG00000087445 | −1.629934605 | 4.98E−05 | 0.001058506 | 21.93782218 | 12.62590526 | 15.61084256 |
| ENSMUSG00000087595 | −1.356715257 | 0.000782501 | 0.008272069 | 15.95477976 | 21.3669166 | 17.44741228 |
| ENSMUSG00000087613 | 1.300460925 | 0.002649524 | 0.020230437 | 53.84738171 | 60.21585586 | 82.6456371 |
| ENSMUSG00000087616 | −2.549514093 | 7.92E−06 | 0.000238545 | 14.95760603 | 13.59712874 | 8.26456371 |
| ENSMUSG00000087658 | −1.652423913 | 0.001338341 | 0.012240744 | 12.96325856 | 5.82734089 | 9.182848567 |
| ENSMUSG00000089726 | −1.506918387 | 4.30E−05 | 0.000942053 | 73.79085641 | 59.24463238 | 45.91424283 |
| ENSMUSG00000089943 | 1.766690487 | 7.03E−13 | 1.62E−10 | 3075.2838 | 2604.821378 | 3147.880489 |
| ENSMUSG00000090021 | −1.656392969 | 0.002988368 | 0.021957099 | 5.983042412 | 7.769787853 | 19.28398199 |
| ENSMUSG00000090145 | 1.252091278 | 1.05E−06 | 4.56E−05 | 514.5416474 | 669.1729788 | 606.0680054 |
| ENSMUSG00000090175 | 1.660039728 | 0.006419999 | 0.037574902 | 840.6174589 | 1098.453758 | 415.0647552 |
| ENSMUSG00000090264 | −2.18334921 | 1.38E−07 | 8.18E−06 | 7.977389882 | 12.62590526 | 21.1205517 |
| ENSMUSG00000090369 | 1.713925914 | 3.56E−08 | 2.62E−06 | 82.76542003 | 126.2590526 | 106.5210434 |
| ENSMUSG00000090555 | −2.901973235 | 9.10E−10 | 1.01E−07 | 121.6551957 | 110.7194769 | 103.7661888 |
| ENSMUSG00000090610 | 1.076721434 | 0.00270073 | 0.020438322 | 79.77389882 | 53.41729149 | 47.75081255 |
| ENSMUSG00000090698 | −2.55687976 | 1.21E−08 | 1.04E−06 | 20.94064844 | 19.42446963 | 24.79369113 |
| ENSMUSG00000091021 | −1.527245218 | 0.003008341 | 0.022044462 | 10.96891109 | 16.51079919 | 18.36569713 |
| ENSMUSG00000091509 | −1.002937779 | 0.000805178 | 0.008420412 | 69.80216147 | 64.10074979 | 68.87136425 |
| ENSMUSG00000092075 | −5.716673555 | 4.92E−16 | 1.97E−13 | 2.991521206 | 2.913670445 | 0 |
| ENSMUSG00000094410 | 1.220382244 | 0.001002234 | 0.009882478 | 53.84738171 | 132.0863935 | 116.6221768 |
| ENSMUSG00000095280 | −1.650205335 | 4.03E−05 | 0.000893784 | 58.83325038 | 25.25181052 | 24.79369113 |
| ENSMUSG00000095351 | −3.092804449 | 0.006028299 | 0.035789253 | 6.980216147 | 60.21585586 | 166.2095591 |
| ENSMUSG00000096833 | 3.208484861 | 0.004128622 | 0.02736411 | 10.96891109 | 124.3166056 | 23.87540627 |
| ENSMUSG00000096910 | 1.124675865 | 0.006036 | 0.035819378 | 64.81629279 | 67.01442023 | 72.54450368 |
| ENSMUSG00000096954 | 1.206343759 | 0.003120612 | 0.022529545 | 33.903907 | 87.41011335 | 81.72735225 |
| ENSMUSG00000097124 | 2.16107918 | 3.03E−13 | 7.50E−11 | 133.6212805 | 103.9209125 | 130.3964497 |
| ENSMUSG00000097221 | 1.550011559 | 0.000809985 | 0.008457711 | 46.86716556 | 44.67628015 | 42.24110341 |
| ENSMUSG00000097312 | −1.433041605 | 0.000236458 | 0.003456142 | 49.85868677 | 34.96404534 | 33.9765397 |
| ENSMUSG00000097536 | 1.179506676 | 0.003789911 | 0.025815692 | 36.89542821 | 55.35973845 | 40.40453369 |
| ENSMUSG00000097615 | −1.278009058 | 7.24E−05 | 0.001413729 | 33.903907 | 25.25181052 | 25.71197599 |
| ENSMUSG00000097660 | −2.932543755 | 3.83E−06 | 0.000131806 | 4.985868677 | 4.856117408 | 6.427993997 |
| ENSMUSG00000097691 | 1.274551418 | 4.53E−08 | 3.23E−06 | 235.3330015 | 259.3166696 | 175.3924076 |
| ENSMUSG00000097743 | −1.078551643 | 0.000185309 | 0.002900208 | 55.84172918 | 55.35973845 | 50.50566712 |
| ENSMUSG00000097908 | 1.156232879 | 5.54E−05 | 0.001144881 | 154.561929 | 95.1799012 | 98.25647967 |
| ENSMUSG00000097971 | −1.77227667 | 3.92E−13 | 9.56E−11 | 2017.282467 | 2103.670061 | 1313.147345 |
| ENSMUSG00000097994 | −1.156597047 | 0.008206265 | 0.04482173 | 14.95760603 | 25.25181052 | 11.93770314 |
| ENSMUSG00000098041 | −1.469996023 | 0.000332181 | 0.004498467 | 22.93499591 | 20.39569311 | 17.44741228 |
| ENSMUSG00000098661 | −1.627002493 | 0.000277688 | 0.003922434 | 19.94347471 | 6.798564371 | 14.69255771 |
| ENSMUSG00000098814 | −4.705165719 | 0.001128634 | 0.010794205 | 2.991521206 | 1.942446963 | 1.836569713 |
| ENSMUSG00000098882 | 1.905068974 | 0.000139968 | 0.002360908 | 128.6354119 | 27.19425749 | 93.66505538 |
| ENSMUSG00000099568 | −2.251364762 | 0.000320113 | 0.004365382 | 1.994347471 | 4.856117408 | 19.28398199 |
| ENSMUSG00000099858 | 1.088333588 | 4.80E−05 | 0.001024996 | 154.561929 | 202.0144842 | 168.0461288 |
| ENSMUSG00000100094 | 1.758512646 | 5.46E−23 | 6.28E−20 | 3175.001173 | 2305.684545 | 3195.631301 |
| ENSMUSG00000100468 | 1.995883006 | 0.000292618 | 0.004105376 | 17.94912724 | 36.9064923 | 34.89482455 |
| ENSMUSG00000101939 | −1.264629649 | 0.000784437 | 0.008279693 | 14.95760603 | 28.16548097 | 21.1205517 |
| ENSMUSG00000102275 | −1.950030022 | 0.000225327 | 0.003321924 | 4.985868677 | 13.59712874 | 14.69255771 |
| ENSMUSG00000102577 | −1.649520189 | 1.70E−08 | 1.42E−06 | 40.88412315 | 64.10074979 | 82.6456371 |
| ENSMUSG00000102719 | −1.730894623 | 0.006769906 | 0.039102589 | 12.96325856 | 5.82734089 | 4.591424283 |
| ENSMUSG00000102869 | 1.362067771 | 5.85E−06 | 0.000185032 | 292.1719044 | 335.0721012 | 361.8042335 |
| ENSMUSG00000102882 | 2.19227753 | 3.68E−07 | 1.87E−05 | 88.74846244 | 63.12952631 | 216.7152262 |
| ENSMUSG00000102918 | 1.26420677 | 0.002814898 | 0.021114835 | 30.91238579 | 47.5899506 | 66.11650968 |
| ENSMUSG00000103285 | −1.692405475 | 0.00269655 | 0.020438322 | 9.971737353 | 5.82734089 | 8.26456371 |
| ENSMUSG00000103546 | −1.97976669 | 0.000414043 | 0.005267075 | 6.980216147 | 6.798564371 | 6.427993997 |
| ENSMUSG00000104030 | −3.028455317 | 2.18E−07 | 1.20E−05 | 3.988694941 | 4.856117408 | 5.50970914 |
| ENSMUSG00000104388 | −2.61061807 | 5.15E−05 | 0.001078975 | 6.980216147 | 1.942446963 | 6.427993997 |
| ENSMUSG00000104399 | −1.177892321 | 0.00272173 | 0.020574411 | 23.93216965 | 29.13670445 | 18.36569713 |
| ENSMUSG00000104445 | 1.012908858 | 0.003001159 | 0.022015494 | 68.80498774 | 51.47484453 | 89.99191596 |
| ENSMUSG00000104973 | −2.007621104 | 9.36E−05 | 0.001719883 | 6.980216147 | 13.59712874 | 4.591424283 |
| ENSMUSG00000105161 | −1.279752452 | 0.00691512 | 0.039789659 | 21.93782218 | 8.741011335 | 13.77427285 |
| ENSMUSG00000105434 | −2.02241612 | 0.000742647 | 0.007975668 | 5.983042412 | 7.769787853 | 6.427993997 |
| ENSMUSG00000105547 | −2.707072316 | 4.65E−06 | 0.000153843 | 5.983042412 | 2.913670445 | 8.26456371 |
| ENSMUSG00000105556 | 1.504518371 | 0.00309422 | 0.022420764 | 21.93782218 | 29.13670445 | 33.05825484 |
| ENSMUSG00000105703 | −1.801332207 | 3.38E−12 | 6.40E−10 | 242.3132177 | 122.3741587 | 160.6998499 |
| ENSMUSG00000105881 | 1.259909682 | 7.19E−05 | 0.001410701 | 247.2990864 | 245.7195409 | 252.5283356 |
| ENSMUSG00000105906 | −2.671616891 | 0.003078263 | 0.022366209 | 6.980216147 | 20.39569311 | 14.69255771 |
| ENSMUSG00000106030 | 1.145150269 | 5.54E−05 | 0.001144881 | 109.6891109 | 147.6259692 | 198.349529 |
| ENSMUSG00000106664 | −1.606191029 | 0.000152255 | 0.002513686 | 9.971737353 | 17.48202267 | 19.28398199 |
| ENSMUSG00000106705 | −2.05446715 | 2.41E−09 | 2.41E−07 | 18.94630097 | 32.05037489 | 31.22168513 |
| ENSMUSG00000106706 | −1.806072384 | 0.002749571 | 0.020715964 | 9.971737353 | 8.741011335 | 4.591424283 |
| ENSMUSG00000106943 | −1.230168101 | 0.009404129 | 0.049324655 | 9.971737353 | 13.59712874 | 13.77427285 |
| ENSMUSG00000107168 | −2.535260536 | 5.76E−05 | 0.00118059 | 1.994347471 | 9.712234816 | 5.50970914 |
| ENSMUSG00000107225 | −3.314781204 | 7.31E−09 | 6.61E−07 | 3.988694941 | 1.942446963 | 6.427993997 |
| ENSMUSG00000107304 | −1.772473058 | 0.001145745 | 0.010886341 | 7.977389882 | 10.6834583 | 11.93770314 |
| ENSMUSG00000107390 | −1.580957843 | 0.000625578 | 0.007061263 | 12.96325856 | 23.30936356 | 11.93770314 |
| ENSMUSG00000107624 | −2.865902972 | 2.57E−10 | 3.34E−08 | 8.974563618 | 13.59712874 | 11.93770314 |
| ENSMUSG00000108368 | −1.782724603 | 5.57E−05 | 0.001147878 | 20.94064844 | 11.65468178 | 7.346278854 |
| ENSMUSG00000108633 | −1.682864395 | 0.001365295 | 0.012400579 | 8.974563618 | 12.62590526 | 9.182848567 |
| ENSMUSG00000108820 | −1.905612833 | 0.000888445 | 0.00904159 | 6.980216147 | 3.884893927 | 8.26456371 |
| ENSMUSG00000108825 | 1.256782178 | 0.000111922 | 0.001987343 | 104.7032422 | 117.5180413 | 191.0032502 |
| ENSMUSG00000109089 | −1.779914445 | 1.06E−06 | 4.61E−05 | 42.87847062 | 34.96404534 | 96.41990995 |
| ENSMUSG00000109115 | 2.077699358 | 0.000502824 | 0.006083704 | 26.92369085 | 27.19425749 | 19.28398199 |
| ENSMUSG00000109157 | −1.885864825 | 0.000870552 | 0.00892677 | 4.985868677 | 6.798564371 | 8.26456371 |
| ENSMUSG00000109262 | 1.329001341 | 0.002840836 | 0.0212393 | 48.86151303 | 45.64750364 | 24.79369113 |
| ENSMUSG00000109291 | −1.949878064 | 0.001232984 | 0.01148511 | 1.994347471 | 9.712234816 | 9.182848567 |
| ENSMUSG00000109536 | −1.039899156 | 0.001827878 | 0.015463254 | 63.81911906 | 37.87771578 | 29.38511541 |
| ENSMUSG00000109555 | −1.255499819 | 0.007385438 | 0.041635066 | 10.96891109 | 14.56835222 | 15.61084256 |
| ENSMUSG00000109807 | −2.757236067 | 2.47E−09 | 2.44E−07 | 11.96608482 | 5.82734089 | 12.85598799 |
| ENSMUSG00000109836 | 1.535086391 | 0.000730915 | 0.007885666 | 46.86716556 | 68.9568672 | 89.0736311 |
| ENSMUSG00000109841 | 1.421032277 | 0.000627493 | 0.007061263 | 46.86716556 | 57.30218542 | 33.05825484 |
| ENSMUSG00000110588 | −5.501119532 | 2.73E−06 | 0.000102051 | 31.90955953 | 14.56835222 | 3.673139427 |
| ENSMUSG00000110613 | 1.168711997 | 0.002147567 | 0.017360028 | 64.81629279 | 58.2734089 | 38.56796398 |
| ENSMUSG00000110702 | −1.389252147 | 0.004080864 | 0.027194141 | 18.94630097 | 11.65468178 | 8.26456371 |
| ENSMUSG00000110755 | 1.852337964 | 4.42E−06 | 0.000147317 | 194.4488784 | 92.26623075 | 176.3106925 |
| ENSMUSG00000111282 | −1.321989153 | 0.002140143 | 0.017319005 | 21.93782218 | 18.45324615 | 30.30340027 |
| ENSMUSG00000111312 | 1.254957582 | 0.001977069 | 0.016387107 | 45.86999182 | 33.99282186 | 56.93366111 |
| ENSMUSG00000111631 | −1.948317608 | 1.41E−05 | 0.000376102 | 11.96608482 | 15.53957571 | 11.01941828 |
| ENSMUSG00000111709 | 2.018073354 | 0.00211263 | 0.017179449 | 31.90955953 | 69.92809068 | 7.346278854 |
| ENSMUSG00000111774 | 2.335615682 | 0.000501244 | 0.006070575 | 17.94912724 | 15.53957571 | 39.48624884 |

| ID | agedliverpbs1 | agedliverpbs2 | agedliverpbs3 | Gene.name |
|---|---|---|---|---|
| ENSMUSG00000000204 | 61.79909158 | 46.97322185 | 22.5130033 | Slfn4 |
| ENSMUSG00000000317 | 92.69863737 | 140.9196656 | 205.5535084 | Bcl6b |
| ENSMUSG00000000686 | 171.5457542 | 161.796653 | 294.6266953 | Abhd15 |
| ENSMUSG00000001227 | 171.5457542 | 203.550628 | 184.9981575 | Sema6b |
| ENSMUSG00000001403 | 51.14407579 | 37.57857748 | 17.61887214 | Ube2c |
| ENSMUSG00000001983 | 128.9256911 | 122.1303768 | 219.2570756 | Taco1 |
| ENSMUSG00000002233 | 330.3054895 | 288.1024274 | 298.5420002 | Rhoc |
| ENSMUSG00000002250 | 627.58043 | 657.6251059 | 647.9829644 | Ppard |
| ENSMUSG00000002289 | 1865.693265 | 2130.496573 | 2428.467877 | Angptl4 |
| ENSMUSG00000002831 | 228.0173379 | 574.117156 | 226.1088592 | Plin4 |
| ENSMUSG00000003032 | 155.5632305 | 107.5164856 | 114.5226689 | Klf4 |
| ENSMUSG00000003348 | 13.85152053 | 30.27163186 | 33.28009183 | Mob3a |
| ENSMUSG00000003500 | 174.742259 | 99.16569058 | 90.05201318 | Impdh1 |
| ENSMUSG00000003541 | 105.4846563 | 45.92937248 | 44.04718036 | Ier3 |
| ENSMUSG00000003848 | 522.0957737 | 291.2339755 | 260.3677772 | Nob1 |
| ENSMUSG00000004100 | 1249.833352 | 729.6507128 | 351.3986167 | Ppan |
| ENSMUSG00000004933 | 42.62006316 | 14.61389124 | 19.57652461 | Matk |
| ENSMUSG00000004951 | 237.6068521 | 636.7481184 | 278.9654756 | Hspb1 |
| ENSMUSG00000005148 | 19.17902842 | 27.14008374 | 22.5130033 | Klf5 |
| ENSMUSG00000005547 | 5271.036311 | 4402.956662 | 4447.78639 | Cyp2a5 |
| ENSMUSG00000005580 | 86.3056279 | 93.9464437 | 169.3369378 | Adcy9 |
| ENSMUSG00000006050 | 2542.286767 | 2021.936238 | 1365.462591 | Sra1 |
| ENSMUSG00000006134 | 225.8863347 | 252.6115486 | 375.8692724 | Crkl |
| ENSMUSG00000006517 | 1097.466626 | 579.3364028 | 613.7240464 | Mvd |
| ENSMUSG00000006587 | 10.65501579 | 2.087698749 | 4.894131151 | Snai3 |
| ENSMUSG00000006711 | 210.9693126 | 141.9635149 | 177.1675477 | D130043K22Rik |
| ENSMUSG00000006777 | 8.524012632 | 8.350794996 | 10.76708853 | Krt23 |
| ENSMUSG00000008153 | 96.96064369 | 120.0426781 | 78.30609842 | Clstn3 |
| ENSMUSG00000009013 | 329.2399879 | 396.6627623 | 281.9019543 | Dynll1 |
| ENSMUSG00000009633 | 2971.683904 | 3862.242686 | 1956.673634 | G0s2 |
| ENSMUSG00000014547 | 21.31003158 | 36.53472811 | 46.98365905 | Wdfy2 |
| ENSMUSG00000014609 | 14.91702211 | 14.61389124 | 15.66121968 | Chrne |
| ENSMUSG00000015224 | 318.5849721 | 255.7430968 | 158.5698493 | Cyp2j9 |
| ENSMUSG00000015312 | 121.46718 | 59.49941435 | 63.62370497 | Gadd45b |
| ENSMUSG00000016128 | 199.2487953 | 218.1645193 | 304.4149576 | Stard13 |
| ENSMUSG00000016356 | 90.56763421 | 172.2351468 | 124.3109312 | Col20a1 |
| ENSMUSG00000017737 | 38.35805684 | 32.35933061 | 22.5130033 | Mmp9 |
| ENSMUSG00000017868 | 543.4058053 | 586.6433485 | 498.2225512 | Sgk2 |
| ENSMUSG00000018486 | 3.196504737 | 15.65774062 | 6.851783612 | Wnt9b |
| ENSMUSG00000019082 | 7723.820946 | 7053.290223 | 5201.482588 | Slc25a22 |
| ENSMUSG00000019726 | 179.0042653 | 147.1827618 | 268.1983871 | Lyst |
| ENSMUSG00000019737 | 45.8165679 | 15.65774062 | 18.59769837 | Syne4 |
| ENSMUSG00000019883 | 200.3142968 | 230.6907118 | 405.2340593 | Echdc1 |
| ENSMUSG00000020018 | 268.5063979 | 250.5238499 | 170.3157641 | Snrpf |
| ENSMUSG00000020027 | 2138.461669 | 846.5618427 | 589.2533906 | Socs2 |
| ENSMUSG00000020091 | 393.1700826 | 471.8199173 | 1129.56547 | Eif4ebp2 |
| ENSMUSG00000020122 | 2670.146957 | 1807.947117 | 1885.219319 | Egfr |
| ENSMUSG00000020335 | 24.50653632 | 41.75397498 | 32.3012656 | Zfp354b |
| ENSMUSG00000020429 | 2643.509417 | 2014.629293 | 3936.839098 | Igfbp1 |
| ENSMUSG00000020441 | 303.66795 | 273.4885361 | 168.3581116 | 2310033P09Rik |
| ENSMUSG00000020532 | 2810.793165 | 1739.053058 | 2484.260972 | Acaca |
| ENSMUSG00000020641 | 408.0871047 | 425.8905448 | 452.2177184 | Rsad2 |
| ENSMUSG00000020656 | 71.38860579 | 76.20100434 | 73.41196727 | Grhl1 |
| ENSMUSG00000020681 | 14.91702211 | 21.92083686 | 12.72474099 | Ace |
| ENSMUSG00000020692 | 249.3273695 | 152.4020087 | 104.7344066 | Nle1 |
| ENSMUSG00000020812 | 180.0697668 | 73.06945621 | 57.75074759 | 1810032O08Rik |
| ENSMUSG00000020889 | 443.2486569 | 371.6103773 | 433.62002 | Nr1d1 |
| ENSMUSG00000020917 | 9135.610538 | 6014.660096 | 10059.39717 | Acly |
| ENSMUSG00000020948 | 9.589514211 | 20.87698749 | 44.04718036 | Klhl28 |
| ENSMUSG00000020961 | 25.5720379 | 35.49087873 | 64.6025312 | Ston2 |
| ENSMUSG00000021250 | 204.5763032 | 115.8672806 | 135.0780198 | Fos |
| ENSMUSG00000021260 | 6.393009474 | 11.48234312 | 10.76708853 | Hhipl1 |
| ENSMUSG00000021416 | 404.8906 | 324.6371555 | 254.4948199 | Eci3 |
| ENSMUSG00000021453 | 979.1959511 | 568.8979091 | 678.3265776 | Gadd45g |
| ENSMUSG00000021611 | 220.5588268 | 227.5591636 | 197.7228985 | Tert |
| ENSMUSG00000021670 | 3051.596522 | 1273.496237 | 2047.704474 | Hmgcr |
| ENSMUSG00000021684 | 54.34058053 | 72.02560684 | 49.92013774 | Pde8b |
| ENSMUSG00000021773 | 44.75106632 | 55.32401685 | 29.36478691 | Comtd1 |
| ENSMUSG00000021775 | 635.0389411 | 274.5323855 | 351.3986167 | Nr1d2 |
| ENSMUSG00000021804 | 7.458511053 | 11.48234312 | 20.55535084 | Rgr |
| ENSMUSG00000021958 | 56.47158369 | 91.85874496 | 47.96248528 | Pinx1 |
| ENSMUSG00000022383 | 440.0521521 | 565.766361 | 794.806899 | Ppara |
| ENSMUSG00000022389 | 599.877389 | 502.0915491 | 601.9781316 | Tef |
| ENSMUSG00000022408 | 38.35805684 | 32.35933061 | 21.53417707 | Fam83f |
| ENSMUSG00000022528 | 643.5629537 | 588.7310472 | 500.1802037 | Hes1 |
| ENSMUSG00000022651 | 52.20957737 | 37.57857748 | 32.3012656 | Retnlg |
| ENSMUSG00000022704 | 291.9474326 | 188.9367368 | 134.0991935 | Qtrt2 |
| ENSMUSG00000022853 | 4279.054341 | 3944.706786 | 4891.194673 | Ehhadh |
| ENSMUSG00000022883 | 166.2182463 | 116.9111299 | 103.7555804 | Robo1 |
| ENSMUSG00000022887 | 350.5500195 | 374.7419254 | 819.2775547 | Masp1 |
| ENSMUSG00000022911 | 105.4846563 | 73.06945621 | 75.36961973 | Arl13b |
| ENSMUSG00000023034 | 572.1743479 | 134.6565693 | 149.7604132 | Nr4a1 |
| ENSMUSG00000023044 | 4109.63959 | 5560.585618 | 7290.297763 | Csad |
| ENSMUSG00000023052 | 36.22705368 | 52.19246872 | 20.55535084 | Npff |
| ENSMUSG00000023067 | 1114.514652 | 1009.402345 | 858.4306039 | Cdkn1a |
| ENSMUSG00000023073 | 369.7290479 | 140.9196656 | 157.5910231 | Slc10a2 |
| ENSMUSG00000023341 | 264.2443916 | 178.498243 | 164.4428067 | Mx2 |
| ENSMUSG00000023571 | 53.27507895 | 58.45556497 | 25.44948199 | C1qtnf12 |
| ENSMUSG00000023800 | 160.8907384 | 217.1206699 | 156.6121968 | Tiam2 |
| ENSMUSG00000023905 | 328.1744863 | 207.7260255 | 109.6285378 | Tnfrsf12a |
| ENSMUSG00000023927 | 61.79909158 | 60.54326372 | 33.28009183 | Satb1 |
| ENSMUSG00000023968 | 20.24453 | 38.62242686 | 33.28009183 | Crip3 |
| ENSMUSG00000024118 | 1463.99917 | 1896.674313 | 1329.246021 | Tedc2 |
| ENSMUSG00000024130 | 1006.898992 | 1228.610714 | 2261.088592 | Abca3 |
| ENSMUSG00000024136 | 64.99559632 | 52.19246872 | 34.25891806 | Dnase1l2 |
| ENSMUSG00000024190 | 1247.702349 | 1464.520672 | 1004.275712 | Dusp1 |
| ENSMUSG00000024236 | 484.8032184 | 345.514143 | 575.5498234 | Svil |
| ENSMUSG00000024411 | 14.91702211 | 13.57004187 | 17.61887214 | Aqp4 |
| ENSMUSG00000024440 | 35.16155211 | 70.98175747 | 72.43314104 | Pcdh12 |
| ENSMUSG00000024665 | 3625.901873 | 3398.773563 | 5157.435407 | Fads2 |
| ENSMUSG00000024843 | 3171.998201 | 2942.611387 | 2249.342677 | Chka |
| ENSMUSG00000024887 | 191.7902842 | 179.5420924 | 296.5843478 | Asah2 |
| ENSMUSG00000024924 | 269.5718995 | 262.006193 | 320.0761773 | Vldlr |
| ENSMUSG00000024970 | 90.56763421 | 63.67481184 | 65.58135743 | Al846148 |
| ENSMUSG00000024978 | 1324.418463 | 791.2378259 | 2079.026913 | Gpam |
| ENSMUSG00000025003 | 152.3667258 | 144.0512137 | 186.95581 | Cyp2c39 |
| ENSMUSG00000025006 | 107.6156595 | 110.6480337 | 207.5111608 | Sorbs1 |
| ENSMUSG00000025153 | 35454.56504 | 23677.63536 | 26074.95195 | Fasn |
| ENSMUSG00000025161 | 61.79909158 | 26.09623436 | 32.3012656 | Slc16a3 |
| ENSMUSG00000025240 | 183.2662716 | 163.8843518 | 418.9376266 | Sacm1l |
| ENSMUSG00000025323 | 71.38860579 | 52.19246872 | 60.68722628 | Sp4 |
| ENSMUSG00000025402 | 646.7594584 | 568.8979091 | 502.1378561 | Nab2 |
| ENSMUSG00000025429 | 188.5937795 | 118.9988287 | 253.5159936 | Pstpip2 |
| ENSMUSG00000025450 | 145.9737163 | 103.3410881 | 105.7132329 | Gm9752 |
| ENSMUSG00000025997 | 39.42355842 | 58.45556497 | 22.5130033 | Ikzf2 |
| ENSMUSG00000026020 | 828.9602284 | 520.8808379 | 466.9001118 | Nop58 |
| ENSMUSG00000026249 | 45.8165679 | 122.1303768 | 117.4591476 | Serpine2 |
| ENSMUSG00000026358 | 104.4191547 | 58.45556497 | 96.9037968 | Rgs1 |
| ENSMUSG00000026398 | 615.8599126 | 631.5288716 | 846.6846892 | Nr5a2 |
| ENSMUSG00000026471 | 181.1352684 | 104.3849374 | 126.2685837 | Mr1 |
| ENSMUSG00000026475 | 1039.929541 | 2826.744106 | 2390.293654 | Rgs16 |
| ENSMUSG00000026525 | 43.68556474 | 19.83313812 | 16.64004591 | Opn3 |
| ENSMUSG00000026822 | 304.7334516 | 223.3837661 | 189.8922887 | Lcn2 |
| ENSMUSG00000026826 | 69.25760263 | 25.05238499 | 13.70356722 | Nr4a2 |
| ENSMUSG00000026832 | 101.22265 | 42.79782435 | 62.64487874 | Cytip |
| ENSMUSG00000027360 | 370.7945495 | 319.4179086 | 181.0828526 | Hdc |
| ENSMUSG00000027398 | 98.02614527 | 82.46410059 | 128.2262362 | Il1b |
| ENSMUSG00000027405 | 920.5933642 | 450.9429298 | 352.3774429 | Nop56 |
| ENSMUSG00000027496 | 55.40608211 | 66.80635997 | 66.56018366 | Aurka |
| ENSMUSG00000027513 | 48173.45739 | 29794.5927 | 39823.54518 | Pck1 |
| ENSMUSG00000027605 | 8380.169919 | 3799.611723 | 6353.561061 | Acss2 |
| ENSMUSG00000027762 | 637.1699442 | 555.3278672 | 690.0724923 | Sucnr1 |
| ENSMUSG00000027907 | 207.7728079 | 117.9549793 | 139.9721509 | S100a11 |
| ENSMUSG00000027947 | 713.8860579 | 763.0538928 | 1358.610808 | Il6ra |
| ENSMUSG00000028008 | 20.24453 | 24.00853561 | 36.21657052 | Asic5 |
| ENSMUSG00000028339 | 295.1439374 | 274.5323855 | 289.7325642 | Col15a1 |
| ENSMUSG00000028445 | 91.63313579 | 280.7954817 | 425.7894102 | Enho |
| ENSMUSG00000028630 | 124.6636847 | 101.2533893 | 226.1088592 | Dyrk2 |
| ENSMUSG00000028838 | 74.58511053 | 49.0609206 | 78.30609842 | Extl1 |
| ENSMUSG00000028859 | 152.3667258 | 96.03414245 | 74.3907935 | Csf3r |
| ENSMUSG00000028862 | 77.78161526 | 40.71012561 | 57.75074759 | Map3k6 |
| ENSMUSG00000028864 | 39.42355842 | 69.93790809 | 111.5861902 | Hgf |
| ENSMUSG00000028957 | 62.86459316 | 53.2363181 | 46.00483282 | Per3 |
| ENSMUSG00000028976 | 42.62006316 | 34.44702936 | 45.02600659 | Slc2a5 |
| ENSMUSG00000029086 | 210.9693126 | 165.9720505 | 243.7277313 | Prom1 |
| ENSMUSG00000029135 | 499.7202405 | 272.4446867 | 294.6266953 | Fosl2 |
| ENSMUSG00000029188 | 186.4627763 | 179.5420924 | 182.0616788 | Slc34a2 |
| ENSMUSG00000029195 | 332.4364926 | 209.8137243 | 422.8529315 | Klb |
| ENSMUSG00000029370 | 190.7247826 | 137.7881174 | 169.3369378 | Rassf6 |
| ENSMUSG00000029373 | 25.5720379 | 28.18393311 | 18.59769837 | Pf4 |
| ENSMUSG00000029380 | 615.8599126 | 454.0744779 | 700.8395809 | Cxcl1 |
| ENSMUSG00000029580 | 16369.30076 | 20515.81561 | 13266.0319 | Actb |
| ENSMUSG00000029591 | 177.9387637 | 131.5250212 | 112.5650165 | Ung |
| ENSMUSG00000029656 | 429.3971363 | 614.8272816 | 554.0156463 | C8b |
| ENSMUSG00000030032 | 43.68556474 | 55.32401685 | 35.23774429 | Wdr54 |
| ENSMUSG00000030055 | 75.65061211 | 74.11330559 | 171.2945903 | Rab43 |
| ENSMUSG00000030691 | 189.6592811 | 180.5859418 | 227.0876854 | Fchsd2 |
| ENSMUSG00000030782 | 115.0741705 | 67.85020934 | 70.47548858 | Tgfb1i1 |
| ENSMUSG00000030814 | 814.0432063 | 869.526529 | 459.069502 | Bcl7c |
| ENSMUSG00000030827 | 120.4016784 | 80.37640184 | 48.94131151 | Fgf21 |
| ENSMUSG00000030934 | 5865.586192 | 5683.759844 | 6052.082582 | Oat |
| ENSMUSG00000030968 | 131.0566942 | 132.5688706 | 93.9673181 | Pdilt |
| ENSMUSG00000031010 | 414.4801142 | 361.1718836 | 775.2303744 | Usp9x |
| ENSMUSG00000031271 | 1418.182602 | 1115.874981 | 1330.224847 | Serpina7 |
| ENSMUSG00000031378 | 429.3971363 | 392.4873648 | 441.4506298 | Abcd1 |
| ENSMUSG00000031465 | 50.07857421 | 30.27163186 | 47.96248528 | Angpt2 |
| ENSMUSG00000031762 | 9477.636545 | 4672.2698 | 1607.23267 | Mt2 |
| ENSMUSG00000031765 | 20312.7221 | 10174.39985 | 4243.211708 | Mt1 |
| ENSMUSG00000032009 | 377.187559 | 338.2071973 | 562.8250824 | Sesn3 |
| ENSMUSG00000032064 | 268.5063979 | 302.7163186 | 381.7422298 | Dixdc1 |
| ENSMUSG00000032083 | 163165.5843 | 112907.9676 | 104422.1611 | Apoa1 |
| ENSMUSG00000032091 | 20.24453 | 8.350794996 | 4.894131151 | Tmprss4 |
| ENSMUSG00000032285 | 26.63753947 | 15.65774062 | 44.04718036 | Dnaja4 |
| ENSMUSG00000032417 | 26.63753947 | 43.84167373 | 17.61887214 | Rwdd2a |
| ENSMUSG00000032418 | 6053.11447 | 3739.068459 | 4136.519649 | Me1 |
| ENSMUSG00000032500 | 75.65061211 | 69.93790809 | 123.332105 | Dclk3 |
| ENSMUSG00000032561 | 453.9036726 | 141.9635149 | 317.1396986 | Acpp |
| ENSMUSG00000032702 | 468.8206947 | 629.4411728 | 864.3035613 | Kank1 |
| ENSMUSG00000032724 | 234.4103474 | 176.4105443 | 323.9914822 | Abtb2 |
| ENSMUSG00000032735 | 436.8556474 | 741.1330559 | 606.8722628 | Ablim3 |
| ENSMUSG00000032786 | 7150.581096 | 4035.521682 | 3301.580875 | Alas1 |
| ENSMUSG00000032849 | 168.3492495 | 143.0073643 | 195.7652461 | Abcc4 |
| ENSMUSG00000032860 | 155.5632305 | 134.6565693 | 141.9298034 | P2ry2 |
| ENSMUSG00000032883 | 856.6632695 | 580.3802522 | 723.3525842 | Acsl3 |
| ENSMUSG00000033105 | 1574.811334 | 996.8761526 | 1567.100795 | Lss |
| ENSMUSG00000033594 | 852.4012632 | 820.4656084 | 539.3332529 | Spata2l |
| ENSMUSG00000033624 | 105.4846563 | 98.1218412 | 178.1463739 | Pdpr |
| ENSMUSG00000033792 | 21.31003158 | 26.09623436 | 43.06835413 | Atp7a |
| ENSMUSG00000033855 | 37.29255526 | 42.79782435 | 77.32727219 | Ston1 |
| ENSMUSG00000033967 | 15.98252368 | 19.83313812 | 39.15304921 | Rnf225 |
| ENSMUSG00000034066 | 96.96064369 | 107.5164856 | 184.9981575 | Farp2 |
| ENSMUSG00000034110 | 55.40608211 | 37.57857748 | 55.79309512 | Kctd7 |
| ENSMUSG00000034271 | 135.3187005 | 37.57857748 | 64.6025312 | Jdp2 |
| ENSMUSG00000034755 | 19.17902842 | 36.53472811 | 20.55535084 | Pcdh11x |
| ENSMUSG00000034765 | 166.2182463 | 53.2363181 | 31.32243937 | Dusp5 |
| ENSMUSG00000034853 | 165.1527447 | 137.7881174 | 147.8027608 | Acot11 |
| ENSMUSG00000034926 | 6118.110066 | 6691.074491 | 12774.66113 | Dhcr24 |
| ENSMUSG00000035078 | 94.82964053 | 101.2533893 | 258.4101248 | Mtmr9 |
| ENSMUSG00000035112 | 43.68556474 | 45.92937248 | 36.21657052 | Wnk4 |
| ENSMUSG00000035164 | 61.79909158 | 70.98175747 | 77.32727219 | Zc3h12c |
| ENSMUSG00000035165 | 144.9082147 | 55.32401685 | 91.03083941 | Kcne3 |
| ENSMUSG00000035284 | 293.0129342 | 218.1645193 | 482.5613315 | Vps13c |
| ENSMUSG00000035900 | 147.0392179 | 160.7528037 | 191.8499411 | Gramd4 |
| ENSMUSG00000035933 | 77.78161526 | 81.42025121 | 127.2474099 | Cog5 |
| ENSMUSG00000035948 | 193.9212874 | 154.4897074 | 418.9376266 | Acss3 |
| ENSMUSG00000036062 | 27.70304105 | 29.22778249 | 12.72474099 | Phf24 |
| ENSMUSG00000036120 | 773.5541463 | 626.3096247 | 392.5093183 | Rfxank |
| ENSMUSG00000036611 | 415.5456158 | 267.2254399 | 309.3090888 | Eepd1 |
| ENSMUSG00000037035 | 141.71171 | 149.2704606 | 192.8287674 | Inhbb |
| ENSMUSG00000037071 | 157237.1335 | 63623.66322 | 106805.6029 | Scd1 |
| ENSMUSG00000037095 | 5413.813523 | 8132.630477 | 6142.134595 | Lrg1 |
| ENSMUSG00000037157 | 11.72051737 | 14.61389124 | 24.47065576 | Il22ra1 |
| ENSMUSG00000037336 | 23.44103474 | 42.79782435 | 31.32243937 | Mfsd2b |
| ENSMUSG00000037443 | 3091.020081 | 2288.117829 | 2056.51391 | Cep85 |
| ENSMUSG00000037447 | 99.09164684 | 54.28016747 | 54.81426889 | Arid5a |
| ENSMUSG00000037465 | 592.4188779 | 817.3340602 | 760.5479809 | Klf10 |
| ENSMUSG00000037583 | 304.7334516 | 631.5288716 | 569.676866 | Nr0b2 |
| ENSMUSG00000037709 | 239.7378553 | 289.1462767 | 357.271574 | Fam13a |
| ENSMUSG00000037887 | 80.97812 | 20.87698749 | 30.34361314 | Dusp8 |
| ENSMUSG00000038217 | 1346.793996 | 1375.793476 | 1970.377202 | Tlcd2 |
| ENSMUSG00000038233 | 284.4889216 | 256.7869461 | 354.3350954 | Fam198a |
| ENSMUSG00000038253 | 112.9431674 | 83.50794996 | 44.04718036 | Hoxa5 |
| ENSMUSG00000038370 | 277.0304105 | 627.3534741 | 676.3689251 | Pcp4l1 |
| ENSMUSG00000038415 | 331.3709911 | 448.855231 | 695.9454497 | Foxq1 |
| ENSMUSG00000038418 | 515.7027642 | 342.3825948 | 912.2660466 | Egr1 |
| ENSMUSG00000038473 | 45.8165679 | 35.49087873 | 46.98365905 | Nos1ap |
| ENSMUSG00000038530 | 64.99559632 | 30.27163186 | 63.62370497 | Rgs4 |
| ENSMUSG00000038583 | 67.12659947 | 41.75397498 | 16.64004591 | Pln |
| ENSMUSG00000038587 | 349.4845179 | 176.4105443 | 253.5159936 | Akap12 |
| ENSMUSG00000038751 | 46.88206947 | 44.8855231 | 44.04718036 | Ptk6 |
| ENSMUSG00000038768 | 106.5501579 | 77.24485371 | 93.9673181 | 9130409I23Rik |
| ENSMUSG00000038774 | 194.986789 | 189.9805862 | 328.8856134 | Ascc3 |
| ENSMUSG00000038844 | 169.4147511 | 146.1389124 | 248.6218625 | Kif16b |
| ENSMUSG00000038895 | 230.1483411 | 226.5153143 | 190.8711149 | Zfp653 |
| ENSMUSG00000039103 | 68.19210105 | 74.11330559 | 43.06835413 | Nexn |
| ENSMUSG00000039304 | 105.4846563 | 73.06945621 | 81.24257711 | Tnfsf10 |
| ENSMUSG00000039533 | 167.2837479 | 54.28016747 | 122.3532788 | Mmd2 |
| ENSMUSG00000039601 | 337.7640005 | 298.5409211 | 280.9231281 | Rcan2 |
| ENSMUSG00000039704 | 59.66808842 | 50.10476998 | 182.0616788 | Lmbrd2 |
| ENSMUSG00000039741 | 105.4846563 | 146.1389124 | 137.0356722 | Bahcc1 |
| ENSMUSG00000039853 | 126.7946879 | 156.5774062 | 258.4101248 | Trim14 |
| ENSMUSG00000039981 | 53.27507895 | 77.24485371 | 62.64487874 | Zc3h12d |
| ENSMUSG00000040093 | 75.65061211 | 69.93790809 | 132.1415411 | Bmf |
| ENSMUSG00000040128 | 3368.050491 | 2671.210549 | 2388.336002 | Pnrc1 |
| ENSMUSG00000040152 | 91.63313579 | 40.71012561 | 85.15788203 | Thbs1 |
| ENSMUSG00000040435 | 430.4626379 | 241.1292055 | 201.6382034 | Ppp1r15a |
| ENSMUSG00000040584 | 118.2706753 | 124.2180756 | 233.939469 | Abcb1a |
| ENSMUSG00000040855 | 259.9823853 | 223.3837661 | 409.1493642 | Reps2 |
| ENSMUSG00000040891 | 1650.461946 | 1922.770548 | 1525.990093 | Foxa3 |
| ENSMUSG00000041134 | 18.11352684 | 31.31548123 | 39.15304921 | Cyyr1 |
| ENSMUSG00000041372 | 9.589514211 | 12.52619249 | 9.788262303 | B4galnt3 |
| ENSMUSG00000041695 | 101.22265 | 78.28870309 | 41.11070167 | Kcnj2 |
| ENSMUSG00000041702 | 59.66808842 | 58.45556497 | 97.88262303 | Btbd7 |
| ENSMUSG00000041920 | 586.0258684 | 355.9526367 | 312.2455675 | Slc16a6 |
| ENSMUSG00000041930 | 157.6942337 | 126.3057743 | 71.45431481 | Fam222a |
| ENSMUSG00000041945 | 3.196504737 | 5.219246872 | 10.76708853 | Mfsd9 |
| ENSMUSG00000042010 | 4018.006454 | 2149.285862 | 2550.821156 | Acacb |
| ENSMUSG00000042115 | 117.2051737 | 124.2180756 | 131.1627149 | Klhdc8a |
| ENSMUSG00000042246 | 77.78161526 | 33.40317998 | 54.81426889 | Tmc7 |
| ENSMUSG00000042333 | 30.89954579 | 27.14008374 | 41.11070167 | Tnfrsf14 |
| ENSMUSG00000042354 | 922.7243674 | 424.8466954 | 389.5728396 | Gnl3 |
| ENSMUSG00000042379 | 140.6462084 | 79.33255246 | 203.5958559 | Esm1 |
| ENSMUSG00000042444 | 255.720379 | 221.2960674 | 319.0973511 | Mindy2 |
| ENSMUSG00000042510 | 94.82964053 | 45.92937248 | 47.96248528 | AA986860 |
| ENSMUSG00000042607 | 37.29255526 | 24.00853561 | 42.0895279 | Asb4 |
| ENSMUSG00000042622 | 89.50213263 | 50.10476998 | 72.43314104 | Maff |
| ENSMUSG00000042680 | 87.37112948 | 114.8234312 | 243.7277313 | Garem1 |
| ENSMUSG00000042743 | 15.98252368 | 14.61389124 | 26.42830822 | Sgtb |
| ENSMUSG00000042745 | 726.6720769 | 889.3596671 | 654.834748 | Id1 |
| ENSMUSG00000043165 | 13.85152053 | 41.75397498 | 38.17422298 | Lor |
| ENSMUSG00000043421 | 138.5152053 | 55.32401685 | 75.36961973 | Hilpda |
| ENSMUSG00000043639 | 20.24453 | 46.97322185 | 49.92013774 | Rbm20 |
| ENSMUSG00000043681 | 543.4058053 | 474.9514654 | 382.721056 | Fam25c |
| ENSMUSG00000044042 | 108.6811611 | 85.59564871 | 203.5958559 | Fmn1 |
| ENSMUSG00000044186 | 17.04802526 | 10.43849374 | 17.61887214 | Nkx2-6 |
| ENSMUSG00000044339 | 61.79909158 | 89.77104621 | 79.28492465 | Alkbh2 |
| ENSMUSG00000044349 | 148.1047195 | 83.50794996 | 50.89896397 | Snhg11 |
| ENSMUSG00000044359 | 69.25760263 | 76.20100434 | 148.781587 | P2ry4 |
| ENSMUSG00000044676 | 609.4669032 | 360.1280342 | 270.1560396 | Zfp612 |
| ENSMUSG00000044749 | 1050.584557 | 874.7457758 | 2096.645785 | Abca6 |
| ENSMUSG00000044948 | 73.51960895 | 52.19246872 | 59.70840005 | Cfap43 |
| ENSMUSG00000045045 | 38.35805684 | 38.62242686 | 56.77192136 | Lrfn4 |
| ENSMUSG00000045294 | 8335.418852 | 7066.860265 | 6456.337815 | Insig1 |
| ENSMUSG00000045348 | 38.35805684 | 44.8855231 | 20.55535084 | Nyap1 |
| ENSMUSG00000045382 | 89.50213263 | 68.89405872 | 29.36478691 | Cxcr4 |
| ENSMUSG00000045411 | 798.0606826 | 403.9697079 | 303.4361314 | 2410002F23Rik |
| ENSMUSG00000045776 | 514.6372626 | 321.5056073 | 424.8105839 | Lrtm1 |
| ENSMUSG00000045875 | 28.76854263 | 45.92937248 | 72.43314104 | Adra1a |
| ENSMUSG00000046541 | 22.37553316 | 22.96468624 | 41.11070167 | Zfp526 |
| ENSMUSG00000046721 | 46.88206947 | 46.97322185 | 42.0895279 | Rpl14-ps1 |
| ENSMUSG00000046908 | 36.22705368 | 28.18393311 | 24.47065576 | Ltb4r1 |
| ENSMUSG00000047496 | 124.6636847 | 234.8661093 | 298.5420002 | Rnf152 |
| ENSMUSG00000047649 | 210.9693126 | 155.5335568 | 111.5861902 | Cd3eap |
| ENSMUSG00000047875 | 49.01307263 | 46.97322185 | 47.96248528 | Gpr157 |
| ENSMUSG00000048191 | 53.27507895 | 40.71012561 | 27.40713445 | Muc6 |
| ENSMUSG00000048644 | 40.48906 | 37.57857748 | 23.49182953 | Ctxn1 |
| ENSMUSG00000048856 | 8878.824658 | 12487.57007 | 11318.1677 | Slc25a47 |
| ENSMUSG00000049044 | 3411.736056 | 3040.733228 | 2890.473858 | Rapgef4 |
| ENSMUSG00000049313 | 152.3667258 | 109.6041843 | 170.3157641 | Sorl1 |
| ENSMUSG00000049580 | 1707.999031 | 1943.647535 | 1562.206663 | Tsku |
| ENSMUSG00000049791 | 215.231319 | 290.1901261 | 450.2600659 | Fzd4 |
| ENSMUSG00000049950 | 94.82964053 | 44.8855231 | 35.23774429 | Rpp38 |
| ENSMUSG00000050390 | 692.5760263 | 634.6604197 | 674.4112726 | C77080 |
| ENSMUSG00000050503 | 25.5720379 | 38.62242686 | 16.64004591 | Fbxl22 |
| ENSMUSG00000050663 | 15.98252368 | 13.57004187 | 15.66121968 | Trhde |
| ENSMUSG00000050737 | 119.3361768 | 55.32401685 | 31.32243937 | Ptges |
| ENSMUSG00000050914 | 273.8339058 | 58.45556497 | 46.98365905 | Ankrd37 |
| ENSMUSG00000051149 | 6.393009474 | 3.131548123 | 21.53417707 | Adnp |
| ENSMUSG00000051339 | 917.3968595 | 914.4120521 | 942.6096597 | 2900026A02Rik |
| ENSMUSG00000051452 | 92.69863737 | 80.37640184 | 127.2474099 | Gm11437 |
| ENSMUSG00000051674 | 295.1439374 | 270.356988 | 333.7797445 | Dcun1d4 |
| ENSMUSG00000051998 | 42.62006316 | 39.66627623 | 21.53417707 | Lax1 |
| ENSMUSG00000052085 | 197.1177921 | 161.796653 | 227.0876854 | Dock8 |
| ENSMUSG00000052595 | 821.5017174 | 661.8005034 | 1485.858218 | A1cf |
| ENSMUSG00000052656 | 1950.933391 | 1650.325861 | 1785.379044 | Rnf103 |
| ENSMUSG00000052684 | 547.6678116 | 268.2692892 | 399.3611019 | Jun |
| ENSMUSG00000052713 | 23.44103474 | 18.78928874 | 42.0895279 | Zfp608 |
| ENSMUSG00000052837 | 1233.850828 | 697.2913822 | 1027.767542 | Junb |
| ENSMUSG00000053560 | 725.6065753 | 569.9417585 | 550.1003414 | Ier2 |
| ENSMUSG00000053964 | 348.4190163 | 621.0903778 | 388.5940134 | Lgals4 |
| ENSMUSG00000053977 | 57.53708526 | 21.92083686 | 40.13187544 | Cd8a |
| ENSMUSG00000054008 | 753.3096163 | 842.3864452 | 2156.354185 | Ndst1 |
| ENSMUSG00000054150 | 33.03054895 | 29.22778249 | 32.3012656 | Syne3 |
| ENSMUSG00000054422 | 57173.74923 | 60050.56682 | 33819.42508 | Fabp1 |
| ENSMUSG00000054453 | 69.25760263 | 36.53472811 | 39.15304921 | Sytl5 |
| ENSMUSG00000054659 | 15.98252368 | 25.05238499 | 65.58135743 | Pm20d2 |
| ENSMUSG00000054932 | 72.45410737 | 72.02560684 | 80.26375088 | Afp |
| ENSMUSG00000055148 | 476.2792058 | 390.3996661 | 293.6478691 | Klf2 |
| ENSMUSG00000055254 | 409.1526063 | 418.5835992 | 554.9944726 | Ntrk2 |
| ENSMUSG00000055491 | 706.4275469 | 442.5921348 | 309.3090888 | Pprc1 |
| ENSMUSG00000055660 | 29.83404421 | 36.53472811 | 60.68722628 | Mettl4 |
| ENSMUSG00000055692 | 90.56763421 | 57.4117156 | 44.04718036 | Tmem191c |
| ENSMUSG00000055980 | 114.0086689 | 159.7089543 | 270.1560396 | Irs1 |
| ENSMUSG00000056054 | 100.1571484 | 98.1218412 | 95.92497057 | S100a8 |
| ENSMUSG00000056071 | 140.6462084 | 203.550628 | 123.332105 | S100a9 |
| ENSMUSG00000056091 | 3671.718441 | 3506.290049 | 4394.929774 | St3gal5 |
| ENSMUSG00000056148 | 23.44103474 | 25.05238499 | 50.89896397 | Rdh9 |
| ENSMUSG00000056313 | 1762.339612 | 990.6130564 | 874.0918236 | Tcim |
| ENSMUSG00000057342 | 564.7158369 | 644.0550641 | 614.7028726 | Sphk2 |
| ENSMUSG00000057604 | 25.5720379 | 28.18393311 | 27.40713445 | Lmcd1 |
| ENSMUSG00000057722 | 323.91248 | 199.3752305 | 166.4004591 | Lepr |
| ENSMUSG00000057969 | 49.01307263 | 42.79782435 | 37.19539675 | Sema3b |
| ENSMUSG00000058207 | 48372.70618 | 79528.79614 | 62218.1105 | Serpina3k |
| ENSMUSG00000058503 | 376.1220574 | 383.0927204 | 227.0876854 | Fam133b |
| ENSMUSG00000058793 | 1397.938072 | 1320.469459 | 1959.610113 | Cds2 |
| ENSMUSG00000058794 | 71.38860579 | 26.09623436 | 30.34361314 | Nfe2 |
| ENSMUSG00000058921 | 261.0478868 | 329.8564023 | 683.2207087 | Slc10a5 |
| ENSMUSG00000059149 | 147.0392179 | 80.37640184 | 237.854774 | Mfsd4a |
| ENSMUSG00000059824 | 24.50653632 | 16.70158999 | 12.72474099 | Dbp |
| ENSMUSG00000060429 | 201.3797984 | 180.5859418 | 380.7634036 | Sntb1 |
| ENSMUSG00000061175 | 495.4582342 | 405.0135573 | 478.6460266 | Fnip2 |
| ENSMUSG00000061292 | 523.1612753 | 399.7943104 | 986.6568401 | Cyp3a59 |
| ENSMUSG00000061436 | 228.0173379 | 186.849038 | 275.0501707 | Hipk2 |
| ENSMUSG00000061536 | 88.43663105 | 94.99029308 | 144.866282i | Sec22c |
| ENSMUSG00000061825 | 298.3404421 | 160.7528037 | 130.1838886 | Ces2c |
| ENSMUSG00000062901 | 297.2749405 | 259.9184942 | 513.8837709 | Klhl24 |
| ENSMUSG00000063535 | 27.70304105 | 29.22778249 | 32.3012656 | Zfp773 |
| ENSMUSG00000063704 | 581.7638621 | 784.9747296 | 446.344761 | Mapk15 |
| ENSMUSG00000063929 | 208.8383095 | 167.0158999 | 174.231069 | Cyp4a32 |
| ENSMUSG00000065126 | 154.497729 | 158.6651049 | 77.32727219 | Snord104 |
| ENSMUSG00000065147 | 54.34058053 | 32.35933061 | 19.57652461 | Snora31 |
| ENSMUSG00000065952 | 51.14407579 | 55.32401685 | 76.34844596 | C330021F23Rik |
| ENSMUSG00000066456 | 70.32310421 | 34.44702936 | 38.17422298 | Hmgn3 |
| ENSMUSG00000066477 | 93.76413895 | 118.9988287 | 119.416800i | Gm16551 |
| ENSMUSG00000066687 | 472.0171995 | 943.6398345 | 1137.39608 | Zbtb16 |
| ENSMUSG00000066944 | 10.65501579 | 10.43849374 | 24.47065576 | NA |
| ENSMUSG00000067149 | 437.921149 | 878.9211733 | 284.838433 | Jchain |
| ENSMUSG00000068463 | 46.88206947 | 15.65774062 | 12.72474099 | B630019A10Rik |
| ENSMUSG00000068742 | 312.1919626 | 260.9623436 | 334.7585707 | Cry2 |
| ENSMUSG00000068877 | 201.3797984 | 256.7869461 | 255.473646i | Selenbp2 |
| ENSMUSG00000069456 | 2190.671246 | 1455.126028 | 1756.014257 | Rdh16 |
| ENSMUSG00000069804 | 42.62006316 | 55.32401685 | 34.25891806 | Gm10277 |
| ENSMUSG00000070576 | 98.02614527 | 132.5688706 | 122.3532788 | Mn1 |
| ENSMUSG00000070583 | 13.85152053 | 11.48234312 | 31.32243937 | Fv1 |
| ENSMUSG00000071076 | 3733.517533 | 2948.874483 | 1847.045097 | Jund |
| ENSMUSG00000071456 | 12.78601895 | 16.70158999 | 35.23774429 | 1110002L01Rik |
| ENSMUSG00000071547 | 90.56763421 | 104.3849374 | 70.47548858 | Nt5dc2 |
| ENSMUSG00000071637 | 239.7378553 | 123.1742262 | 171.2945903 | Cebpd |
| ENSMUSG00000071645 | 605.2048969 | 525.0562354 | 343.5680068 | Tut1 |
| ENSMUSG00000072294 | 24.50653632 | 18.78928874 | 60.68722628 | Klf12 |
| ENSMUSG00000072571 | 20.24453 | 29.22778249 | 29.36478691 | Tmem253 |
| ENSMUSG00000072664 | 273.8339058 | 440.504436 | 724.3314104 | Ugt3a1 |
| ENSMUSG00000072692 | 73.51960895 | 70.98175747 | 58.72957382 | Rpl37rt |
| ENSMUSG00000072849 | 570.0433447 | 342.3825948 | 378.8057511 | Serpina1e |
| ENSMUSG00000072999 | 294.0784358 | 417.5397498 | 310.287915 | Gm15401 |
| ENSMUSG00000073460 | 51.14407579 | 79.33255246 | 37.19539675 | Pnldc1 |
| ENSMUSG00000073835 | 85.24012632 | 69.93790809 | 16.64004591 | Mup-ps12 |
| ENSMUSG00000074024 | 118.2706753 | 117.9549793 | 98.86144926 | 4632427E13Rik |
| ENSMUSG00000074063 | 2843.823714 | 6567.900264 | 5870.020903 | Osgin1 |
| ENSMUSG00000074213 | 24.50653632 | 27.14008374 | 18.59769837 | Gm10642 |
| ENSMUSG00000074345 | 26.63753947 | 24.00853561 | 29.36478691 | Tnfaip8l3 |
| ENSMUSG00000074375 | 860.9252758 | 876.8334746 | 855.4941252 | Sult2a3 |
| ENSMUSG00000074876 | 71.38860579 | 41.75397498 | 54.81426889 | Spata5l1 |
| ENSMUSG00000075470 | 77.78161526 | 80.37640184 | 251.5583412 | Deaf1 |
| ENSMUSG00000075552 | 1023.947017 | 947.815232 | 894.6471745 | Cyp3a41b |
| ENSMUSG00000075590 | 1682.426993 | 1456.169877 | 1087.475942 | Nrbp2 |
| ENSMUSG00000076490 | 35.16155211 | 20.87698749 | 18.59769837 | Trbc1 |
| ENSMUSG00000076569 | 72.45410737 | 110.6480337 | 183.0405051 | Igkv5-39 |
| ENSMUSG00000076596 | 152.3667258 | 28.18393311 | 26.42830822 | Igkv3-10 |
| ENSMUSG00000076609 | 4135.211628 | 4467.675323 | 1384.06029 | Igkc |
| ENSMUSG00000076613 | 100.1571484 | 155.5335568 | 99.84027549 | Ighg2b |
| ENSMUSG00000076617 | 5037.691465 | 5276.658588 | 2480.345667 | Ighm |
| ENSMUSG00000076934 | 77.78161526 | 36.53472811 | 58.72957382 | Iglv1 |
| ENSMUSG00000077148 | 9.589514211 | 19.83313812 | 11.74591476 | Gm22935 |
| ENSMUSG00000078193 | 42.62006316 | 35.49087873 | 30.34361314 | Gm2000 |
| ENSMUSG00000078234 | 197.1177921 | 263.0500424 | 351.3986167 | Klhdc7a |
| ENSMUSG00000078650 | 6922.563759 | 10279.82864 | 10371.64274 | G6pc |
| ENSMUSG00000078651 | 55.40608211 | 57.4117156 | 43.06835413 | Aoc2 |
| ENSMUSG00000078672 | 337.7640005 | 374.7419254 | 237.854774 | Mup20 |
| ENSMUSG00000078688 | 49.01307263 | 38.62242686 | 39.15304921 | Mup2 |
| ENSMUSG00000078817 | 204.5763032 | 280.7954817 | 342.5891806 | Nlrp12 |
| ENSMUSG00000079017 | 145.9737163 | 124.2180756 | 88.09436072 | Ifi27l2a |
| ENSMUSG00000079036 | 575.3708526 | 288.1024274 | 180.1040264 | Alkbh1 |
| ENSMUSG00000079065 | 371.8600511 | 353.864938 | 194.7864198 | BC005561 |
| ENSMUSG00000079465 | 46.88206947 | 56.36786622 | 36.21657052 | Col4a3 |
| ENSMUSG00000079470 | 201.3797984 | 169.1035987 | 244.7065576 | Utp14b |
| ENSMUSG00000080059 | 37.29255526 | 13.57004187 | 19.57652461 | Rps19-ps3 |
| ENSMUSG00000081344 | 64.99559632 | 55.32401685 | 43.06835413 | Gm14303 |
| ENSMUSG00000082065 | 60.73359 | 39.66627623 | 5.872957382 | Mup-ps14 |
| ENSMUSG00000082173 | 41.55456158 | 31.31548123 | 8.809436072 | Mup-ps10 |
| ENSMUSG00000082586 | 15.98252368 | 7.306945621 | 17.61887214 | Sult2a-ps1 |
| ENSMUSG00000082658 | 85.24012632 | 129.4373224 | 49.92013774 | Fau-ps2 |
| ENSMUSG00000083327 | 36.22705368 | 34.44702936 | 66.56018366 | Vcp-rs |
| ENSMUSG00000083621 | 54.34058053 | 48.01707123 | 31.32243937 | Gm14586 |
| ENSMUSG00000083716 | 70.32310421 | 86.63949808 | 39.15304921 | Gm13436 |
| ENSMUSG00000083813 | 86.3056279 | 84.55179933 | 92.00966564 | Gm15502 |
| ENSMUSG00000083863 | 64.99559632 | 54.28016747 | 47.96248528 | Gm13341 |
| ENSMUSG00000083992 | 39.42355842 | 40.71012561 | 29.36478691 | Gm11478 |
| ENSMUSG00000084822 | 66.0610979 | 91.85874496 | 58.72957382 | Myadml2os |
| ENSMUSG00000084883 | 24.50653632 | 30.27163186 | 63.62370497 | Ccdc85c |
| ENSMUSG00000085001 | 156.6287321 | 70.98175747 | 70.47548858 | Rapgef4os2 |
| ENSMUSG00000085156 | 157.6942337 | 59.49941435 | 34.25891806 | Snhg15 |
| ENSMUSG00000085445 | 126.7946879 | 143.0073643 | 187.9346362 | Gm16348 |
| ENSMUSG00000085834 | 1120.907661 | 744.264604 | 450.2600659 | Gm15622 |
| ENSMUSG00000085995 | 444.3141584 | 772.4485371 | 450.2600659 | Gm2788 |
| ENSMUSG00000086140 | 11.72051737 | 22.96468624 | 25.44948199 | Hnf1aos2 |
| ENSMUSG00000086446 | 27.70304105 | 97.07799183 | 26.42830822 | Prkag2os1 |
| ENSMUSG00000086529 | 13.85152053 | 8.350794996 | 2.936478691 | Acss2os |
| ENSMUSG00000086786 | 2.131003158 | 3.131548123 | 15.66121968 | Gm15908 |
| ENSMUSG00000086844 | 73.51960895 | 42.79782435 | 46.98365905 | B230206H07Rik |
| ENSMUSG00000087382 | 414.4801142 | 369.5226786 | 445.3659348 | Ctcflos |
| ENSMUSG00000087445 | 61.79909158 | 54.28016747 | 39.15304921 | Gm14286 |
| ENSMUSG00000087595 | 41.55456158 | 62.63096247 | 36.21657052 | 1810012K08Rik |
| ENSMUSG00000087613 | 12.78601895 | 17.74543937 | 48.94131151 | Gm13855 |
| ENSMUSG00000087616 | 154.497729 | 34.44702936 | 26.42830822 | Gm14257 |
| ENSMUSG00000087658 | 36.22705368 | 28.18393311 | 23.49182953 | Hotairm1 |
| ENSMUSG00000089726 | 293.0129342 | 129.4373224 | 86.13670826 | Mir17hg |
| ENSMUSG00000089943 | 781.0126574 | 515.661591 | 1296.944755 | Ugt1a5 |
| ENSMUSG00000090021 | 15.98252368 | 39.66627623 | 48.94131151 | Gm6493 |
| ENSMUSG00000090145 | 154.497729 | 220.252218 | 375.8692724 | Ugt1a6b |
| ENSMUSG00000090175 | 125.7291863 | 209.8137243 | 409.1493642 | Ugt1a9 |
| ENSMUSG00000090264 | 57.53708526 | 80.37640184 | 52.85661643 | Eif4ebp3 |
| ENSMUSG00000090369 | 27.70304105 | 27.14008374 | 41.11070167 | 4933411K16Rik |
| ENSMUSG00000090555 | 1396.87257 | 624.2219259 | 491.3707676 | Gm8893 |
| ENSMUSG00000090610 | 35.16155211 | 28.18393311 | 22.5130033 | Gm3571 |
| ENSMUSG00000090698 | 247.1963663 | 60.54326372 | 76.34844596 | Apold1 |
| ENSMUSG00000091021 | 82.04362158 | 27.14008374 | 23.49182953 | Gm17300 |
| ENSMUSG00000091509 | 143.8427132 | 183.7174899 | 79.28492465 | Gm17066 |
| ENSMUSG00000092075 | 83.10912316 | 163.8843518 | 57.75074759 | Serpina4-ps1 |
| ENSMUSG00000094410 | 47.94757105 | 25.05238499 | 56.77192136 | Gm38394 |
| ENSMUSG00000095280 | 126.7946879 | 158.6651049 | 55.79309512 | Gm21738 |
| ENSMUSG00000095351 | 629.7114332 | 1051.15632 | 311.2667412 | Igkv3-2 |
| ENSMUSG00000096833 | 3.196504737 | 4.175397498 | 9.788262303 | Igkv4-55 |
| ENSMUSG00000096910 | 34.09605053 | 10.43849374 | 48.94131151 | Zfp955b |
| ENSMUSG00000096954 | 33.03054895 | 17.74543937 | 37.19539675 | Gdap10 |
| ENSMUSG00000097124 | 23.44103474 | 33.40317998 | 25.44948199 | A530020G20Rik |
| ENSMUSG00000097221 | 8.524012632 | 9.39464437 | 27.40713445 | 1810049J17Rik |
| ENSMUSG00000097312 | 114.0086689 | 161.796653 | 45.02600659 | Gm26870 |
| ENSMUSG00000097536 | 13.85152053 | 26.09623436 | 18.59769837 | 2610037D02Rik |
| ENSMUSG00000097615 | 69.25760263 | 65.76251059 | 70.47548858 | Gm2061 |
| ENSMUSG00000097660 | 60.73359 | 53.2363181 | 10.76708853 | Gm26762 |
| ENSMUSG00000097691 | 75.65061211 | 98.1218412 | 102.7767542 | 9030616G12Rik |
| ENSMUSG00000097743 | 155.5632305 | 98.1218412 | 88.09436072 | Gm16973 |
| ENSMUSG00000097908 | 52.20957737 | 45.92937248 | 57.75074759 | 4933404O12Rik |
| ENSMUSG00000097971 | 8345.008366 | 6440.550641 | 3776.311596 | Gm26917 |
| ENSMUSG00000097994 | 29.83404421 | 48.01707123 | 38.17422298 | Gm26982 |
| ENSMUSG00000098041 | 68.19210105 | 69.93790809 | 30.34361314 | Gm26981 |
| ENSMUSG00000098661 | 43.68556474 | 48.01707123 | 36.21657052 | Mir7052 |
| ENSMUSG00000098814 | 13.85152053 | 157.6212555 | 4.894131151 | Igkv19-93 |
| ENSMUSG00000098882 | 37.29255526 | 18.78928874 | 10.76708853 | Mir6392 |
| ENSMUSG00000099568 | 18.11352684 | 59.49941435 | 47.96248528 | Gm28513 |
| ENSMUSG00000099858 | 56.47158369 | 75.15715496 | 114.5226689 | Gm6652 |
| ENSMUSG00000100094 | 1017.554008 | 731.7384115 | 815.3622498 | 1810008l18Rik |
| ENSMUSG00000100468 | 6.393009474 | 6.263096247 | 9.788262303 | Tmem167-ps1 |
| ENSMUSG00000101939 | 52.20957737 | 56.36786622 | 46.00483282 | Gm28438 |
| ENSMUSG00000102275 | 56.47158369 | 54.28016747 | 18.59769837 | Gm37144 |
| ENSMUSG00000102577 | 137.4497037 | 252.6115486 | 199.680551 | Gm37969 |
| ENSMUSG00000102719 | 38.35805684 | 30.27163186 | 8.809436072 | Gm37760 |
| ENSMUSG00000102869 | 86.3056279 | 81.42025121 | 216.3205969 | 2900097C17Rik |
| ENSMUSG00000102882 | 26.63753947 | 38.62242686 | 15.66121968 | Gm2065 |
| ENSMUSG00000102918 | 18.11352684 | 14.61389124 | 27.40713445 | Pcdhgc3 |
| ENSMUSG00000103285 | 27.70304105 | 35.49087873 | 14.68239345 | Gm37274 |
| ENSMUSG00000103546 | 28.76854263 | 33.40317998 | 17.61887214 | Gm37666 |
| ENSMUSG00000104030 | 56.47158369 | 39.66627623 | 21.53417707 | 5330406M23Rik |
| ENSMUSG00000104388 | 46.88206947 | 34.44702936 | 12.72474099 | Gm37033 |
| ENSMUSG00000104399 | 77.78161526 | 41.75397498 | 42.0895279 | Gm37963 |
| ENSMUSG00000104445 | 38.35805684 | 40.71012561 | 25.44948199 | Rhbg |
| ENSMUSG00000104973 | 36.22705368 | 32.35933061 | 32.3012656 | A530041M06Rik |
| ENSMUSG00000105161 | 44.75106632 | 39.66627623 | 23.49182953 | Gm42595 |
| ENSMUSG00000105434 | 26.63753947 | 42.79782435 | 12.72474099 | Gm43359 |
| ENSMUSG00000105547 | 23.44103474 | 27.14008374 | 61.66605251 | Iglc3 |
| ENSMUSG00000105556 | 7.458511053 | 9.39464437 | 12.72474099 | Gm43080 |
| ENSMUSG00000105703 | 712.8205563 | 708.7737253 | 409.1493642 | Gm43305 |
| ENSMUSG00000105881 | 43.68556474 | 106.4726362 | 160.5275018 | 4932422M17Rik |
| ENSMUSG00000105906 | 182.20077 | 62.63096247 | 23.49182953 | Iglc1 |
| ENSMUSG00000106030 | 62.86459316 | 88.72719683 | 54.81426889 | Gm43611 |
| ENSMUSG00000106664 | 54.34058053 | 55.32401685 | 33.28009183 | Gm17936 |
| ENSMUSG00000106705 | 87.37112948 | 163.8843518 | 91.03083941 | Gm2602 |
| ENSMUSG00000106706 | 31.96504737 | 38.62242686 | 10.76708853 | C530043K16Rik |
| ENSMUSG00000106943 | 30.89954579 | 34.44702936 | 22.5130033 | Dancr |
| ENSMUSG00000107168 | 46.88206947 | 39.66627623 | 13.70356722 | Gm42507 |
| ENSMUSG00000107225 | 46.88206947 | 43.84167373 | 33.28009183 | Gm43637 |
| ENSMUSG00000107304 | 28.76854263 | 59.49941435 | 16.64004591 | Gm43775 |
| ENSMUSG00000107390 | 46.88206947 | 70.98175747 | 26.42830822 | Gm43323 |
| ENSMUSG00000107624 | 103.3536532 | 111.6918831 | 37.19539675 | Gm44005 |
| ENSMUSG00000108368 | 46.88206947 | 41.75397498 | 47.96248528 | Gm45053 |
| ENSMUSG00000108633 | 49.01307263 | 33.40317998 | 16.64004591 | Gm44694 |
| ENSMUSG00000108820 | 21.31003158 | 25.05238499 | 25.44948199 | Gm44620 |
| ENSMUSG00000108825 | 37.29255526 | 51.14861935 | 84.1790558 | Gm45838 |
| ENSMUSG00000109089 | 112.9431674 | 204.5944774 | 281.9019543 | 4833411C07Rik |
| ENSMUSG00000109115 | 4.262006316 | 4.175397498 | 8.809436072 | Gm44669 |
| ENSMUSG00000109157 | 25.5720379 | 30.27163186 | 18.59769837 | Gm44829 |
| ENSMUSG00000109262 | 14.91702211 | 19.83313812 | 12.72474099 | Gm44744 |
| ENSMUSG00000109291 | 17.04802526 | 38.62242686 | 25.44948199 | Gm2814 |
| ENSMUSG00000109536 | 109.7466626 | 90.81489558 | 68.51783612 | 9330162G02Rik |
| ENSMUSG00000109555 | 43.68556474 | 3.40317998 | 21.53417707 | Gm44891 |
| ENSMUSG00000109807 | 75.65061211 | 94.99029308 | 37.19539675 | Gm45244 |
| ENSMUSG00000109836 | 12.78601895 | 12.52619249 | 45.02600659 | Gm45884 |
| ENSMUSG00000109841 | 13.85152053 | 15.65774062 | 21.53417707 | E330011O21Rik |
| ENSMUSG00000110588 | 1747.42259 | 289.1462767 | 231.9818166 | Gm45774 |
| ENSMUSG00000110613 | 26.63753947 | 16.70158999 | 28.38596068 | Lncbate1 |
| ENSMUSG00000110702 | 45.8165679 | 30.27163186 | 25.44948199 | Gm45767 |
| ENSMUSG00000110755 | 79.91261842 | 28.18393311 | 20.55535084 | BC049987 |
| ENSMUSG00000111282 | 87.37112948 | 62.63096247 | 27.40713445 | Gm47528 |
| ENSMUSG00000111312 | 13.85152053 | 20.87698749 | 22.5130033 | Gm47205 |
| ENSMUSG00000111631 | 28.76854263 | 66.80635997 | 52.85661643 | Gm32017 |
| ENSMUSG00000111709 | 12.78601895 | 8.350794996 | 5.872957382 | Gm3776 |
| ENSMUSG00000111774 | 5.327507895 | 7.306945621 | 1.957652461 | AC166078.1 |

TABLE 4
RNA-seq analysis of differentially expressed genes between the PBS (Control
Group) or TGFRt15-TGFRs (TGFRt15-TGFRs group) in aged mice liver

| ID | log2FoldChange | pvalue | padj | agedliver92181tox | agedliver92182tox |
|---|---|---|---|---|---|
| ENSMUSG00000000204 | −2.005879896 | 3.88E−05 | 0.000867087 | 8.974563618 | 11.65468178 |
| ENSMUSG00000000317 | −1.462090887 | 1.76E−06 | 7.11E−05 | 60.82759785 | 43.70505667 |
| ENSMUSG00000000686 | 1.151464201 | 2.84E−06 | 0.000104278 | 355.9910235 | 571.0794072 |
| ENSMUSG00000001227 | −1.099312542 | 1.49E−06 | 6.21E−05 | 100.7145473 | 67.98564371 |
| ENSMUSG00000001403 | −1.612087212 | 0.002110809 | 0.017174878 | 16.9519535 | 6.798564371 |
| ENSMUSG00000001983 | 1.013926416 | 1.20E−05 | 0.000332241 | 308.1266842 | 343.8131125 |
| ENSMUSG00000002233 | −1.270303057 | 1.05E−10 | 1.46E−08 | 133.6212805 | 104.892136 |
| ENSMUSG00000002250 | −1.142106413 | 9.77E−06 | 0.000282853 | 303.1408155 | 156.3669805 |
| ENSMUSG00000002289 | −2.433615292 | 5.35E−35 | 1.82E−31 | 340.0362437 | 320.5037489 |
| ENSMUSG00000002831 | −2.428567233 | 6.40E−14 | 1.90E−11 | 67.807814 | 71.87053764 |
| ENSMUSG00000003032 | −1.814709673 | 6.30E−10 | 7.35E−08 | 33.903907 | 28.16548097 |
| ENSMUSG00000003348 | 1.035062446 | 0.006673687 | 0.038727262 | 47.86433929 | 51.47484453 |
| ENSMUSG00000003500 | −1.169914769 | 9.39E−05 | 0.001721093 | 55.84172918 | 53.41729149 |
| ENSMUSG00000003541 | −2.579058324 | 2.05E−08 | 1.65E−06 | 9.971737353 | 11.65468178 |
| ENSMUSG00000003848 | −1.114660438 | 1.53E−05 | 0.000400378 | 168.5223613 | 191.3310259 |
| ENSMUSG00000004100 | −2.152000208 | 2.40E−05 | 0.000578522 | 177.4969249 | 181.6187911 |
| ENSMUSG00000004933 | −1.661199536 | 0.00641801 | 0.037574902 | 11.96608482 | 8.741011335 |
| ENSMUSG00000004951 | −1.245761383 | 7.16E−05 | 0.001407433 | 177.4969249 | 194.2446963 |
| ENSMUSG00000005148 | −1.641216558 | 0.00407345 | 0.027163637 | 8.974563618 | 3.884893927 |
| ENSMUSG00000005547 | 1.316837452 | 9.30E−15 | 3.02E−12 | 9129.125547 | 13948.71164 |
| ENSMUSG00000005580 | 1.4493211 | 2.75E−08 | 2.14E−06 | 248.2962601 | 371.00737 |
| ENSMUSG00000006050 | −1.042916307 | 2.32E−07 | 1.27E−05 | 992.1878666 | 1015.899762 |
| ENSMUSG00000006134 | 1.201348738 | 1.44E−09 | 1.52E−07 | 677.0809663 | 610.8995699 |
| ENSMUSG00000006517 | 1.182376585 | 1.39E−07 | 8.18E−06 | 2010.30225 | 1472.374798 |
| ENSMUSG00000006587 | 1.922052303 | 0.003206819 | 0.022932036 | 30.91238579 | 14.56835222 |
| ENSMUSG00000006711 | 1.246016282 | 4.07E−11 | 5.91E−09 | 435.7649223 | 410.8275327 |
| ENSMUSG00000006777 | 1.982368887 | 0.000106088 | 0.001901083 | 22.93499591 | 53.41729149 |
| ENSMUSG00000008153 | 1.444860417 | 1.72E−08 | 1.42E−06 | 278.2114721 | 339.9282186 |
| ENSMUSG00000009013 | −1.025154388 | 6.49E−07 | 3.04E−05 | 138.6071492 | 163.1655449 |
| ENSMUSG00000009633 | −1.150697898 | 7.41E−07 | 3.38E−05 | 1177.662181 | 1721.008009 |
| ENSMUSG00000014547 | 1.47853344 | 5.88E−06 | 0.00018548 | 76.78237762 | 107.8058065 |
| ENSMUSG00000014609 | 1.198939285 | 0.004891473 | 0.030982358 | 34.90108074 | 33.99282186 |
| ENSMUSG00000015224 | −1.47719251 | 7.85E−07 | 3.55E−05 | 57.83607665 | 125.2878291 |
| ENSMUSG00000015312 | −2.170653759 | 1.83E−07 | 1.04E−05 | 27.92086459 | 16.51079919 |
| ENSMUSG00000016128 | 1.194768611 | 1.81E−09 | 1.86E−07 | 486.6207828 | 535.1441384 |
| ENSMUSG00000016356 | −1.303561468 | 6.94E−06 | 0.00021464 | 50.8558605 | 59.24463238 |
| ENSMUSG00000017737 | −1.495519922 | 0.005185903 | 0.032307064 | 19.94347471 | 7.769787853 |
| ENSMUSG00000017868 | 1.520950393 | 2.42E−16 | 1.03E−13 | 1574.537328 | 1919.1376 |
| ENSMUSG00000018486 | 1.561119026 | 0.006908525 | 0.039768921 | 26.92369085 | 21.3669166 |
| ENSMUSG00000019082 | −1.214544808 | 1.93E−12 | 4.05E−10 | 2583.677148 | 3303.131061 |
| ENSMUSG00000019726 | 1.436923585 | 3.39E−10 | 4.31E−08 | 430.7790536 | 552.626161 |
| ENSMUSG00000019737 | −1.672592181 | 0.00414668 | 0.027410702 | 8.974563618 | 9.712234816 |
| ENSMUSG00000019883 | 1.383035463 | 1.73E−09 | 1.79E−07 | 790.7587721 | 685.683778 |
| ENSMUSG00000020018 | −1.224865075 | 5.55E−07 | 2.68E−05 | 76.78237762 | 100.0360186 |
| ENSMUSG00000020027 | −2.148540831 | 0.000201757 | 0.00309142 | 308.1266842 | 206.8706016 |
| ENSMUSG00000020091 | 1.323362559 | 0.005934886 | 0.035409378 | 1482.797344 | 1694.784975 |
| ENSMUSG00000020122 | −1.730853584 | 6.04E−21 | 5.49E−18 | 534.4851221 | 692.4823424 |
| ENSMUSG00000020335 | 1.099463556 | 0.002629472 | 0.020167667 | 41.88129688 | 88.38133683 |
| ENSMUSG00000020429 | −1.914789163 | 0.005628577 | 0.034083881 | 1082.930677 | 219.4965068 |
| ENSMUSG00000020441 | −1.319584205 | 1.17E−07 | 7.17E−06 | 96.72585232 | 114.6043708 |
| ENSMUSG00000020532 | 1.125678465 | 6.99E−06 | 0.000215157 | 6805.710743 | 2936.008585 |
| ENSMUSG00000020641 | −1.035196027 | 2.26E−05 | 0.00055792 | 307.1295105 | 181.6187911 |
| ENSMUSG00000020656 | −2.17958622 | 1.43E−08 | 1.21E−06 | 16.9519535 | 7.769787853 |
| ENSMUSG00000020681 | 1.270736177 | 0.003452779 | 0.024158824 | 30.91238579 | 35.93526882 |
| ENSMUSG00000020692 | −1.373008874 | 7.68E−06 | 0.000232298 | 53.84738171 | 65.07197327 |
| ENSMUSG00000020812 | −1.318469089 | 0.000692163 | 0.007551219 | 32.90673326 | 43.70505667 |
| ENSMUSG00000020889 | 1.857219295 | 5.65E−19 | 3.50E−16 | 1005.151125 | 1714.209445 |
| ENSMUSG00000020917 | 1.579449336 | 7.98E−12 | 1.40E−09 | 34685.69121 | 17873.42573 |
| ENSMUSG00000020948 | 1.248898073 | 0.003935164 | 0.026500655 | 67.807814 | 53.41729149 |
| ENSMUSG00000020961 | 1.570215029 | 7.42E−07 | 3.38E−05 | 116.669327 | 116.5468178 |
| ENSMUSG00000021250 | −3.196150425 | 1.40E−18 | 7.95E−16 | 14.95760603 | 8.741011335 |
| ENSMUSG00000021260 | 1.563177947 | 0.002093122 | 0.017081929 | 31.90955953 | 29.13670445 |
| ENSMUSG00000021416 | 1.171425747 | 1.31E−08 | 1.13E−06 | 910.4196203 | 675.9715432 |
| ENSMUSG00000021453 | −2.706070458 | 5.59E−23 | 6.28E−20 | 149.5760603 | 61.18707934 |
| ENSMUSG00000021611 | 1.027773708 | 1.21E−09 | 1.30E−07 | 416.8186214 | 423.453438 |
| ENSMUSG00000021670 | 1.206582161 | 2.43E−07 | 1.30E−05 | 4843.272832 | 4623.994996 |
| ENSMUSG00000021684 | −1.138260866 | 0.001182935 | 0.011115132 | 32.90673326 | 25.25181052 |
| ENSMUSG00000021773 | −1.294178405 | 0.002327317 | 0.01846284 | 17.94912724 | 12.62590526 |
| ENSMUSG00000021775 | 1.550962326 | 2.41E−10 | 3.16E−08 | 1168.687618 | 1405.360378 |
| ENSMUSG00000021804 | 1.339955479 | 0.00532128 | 0.032924815 | 26.92369085 | 35.93526882 |
| ENSMUSG00000021958 | −1.041099878 | 0.007096785 | 0.040595156 | 25.92651712 | 21.3669166 |
| ENSMUSG00000022383 | 1.183413475 | 3.19E−08 | 2.41E−06 | 1160.710228 | 1260.648079 |
| ENSMUSG00000022389 | 1.619123522 | 2.95E−16 | 1.22E−13 | 1357.153454 | 2316.368004 |
| ENSMUSG00000022408 | 1.528188008 | 2.54E−06 | 9.66E−05 | 74.78803015 | 107.8058065 |
| ENSMUSG00000022528 | −1.557702695 | 1.66E−19 | 1.13E−16 | 205.4177895 | 185.503685 |
| ENSMUSG00000022651 | −4.171544663 | 2.34E−09 | 2.36E−07 | 3.988694941 | 2.913670445 |
| ENSMUSG00000022704 | −1.016808687 | 0.000177103 | 0.002808323 | 94.73150485 | 108.7770299 |
| ENSMUSG00000022853 | 1.04479536 | 2.29E−11 | 3.59E−09 | 9525.00352 | 10073.52995 |
| ENSMUSG00000022883 | 1.330834067 | 1.00E−09 | 1.09E−07 | 280.2058196 | 336.0433246 |
| ENSMUSG00000022887 | 1.363326654 | 1.68E−07 | 9.74E−06 | 1196.608482 | 1241.22361 |
| ENSMUSG00000022911 | 1.024722285 | 3.96E−05 | 0.000880701 | 133.6212805 | 188.4173554 |
| ENSMUSG00000023034 | −2.875895339 | 0.000200077 | 0.003084527 | 58.83325038 | 34.96404534 |
| ENSMUSG00000023044 | −1.350199698 | 7.38E−12 | 1.31E−09 | 1875.683796 | 2461.080302 |
| ENSMUSG00000023052 | −1.334599319 | 0.004914148 | 0.031082668 | 13.96043229 | 11.65468178 |
| ENSMUSG00000023067 | −3.022867976 | 2.66E−57 | 3.63E−53 | 136.6128017 | 95.1799012 |
| ENSMUSG00000023073 | 1.225822592 | 2.02E−05 | 0.00050363 | 443.7423122 | 525.4319036 |
| ENSMUSG00000023341 | −1.14606456 | 1.95E−06 | 7.71E−05 | 89.74563618 | 99.06479513 |
| ENSMUSG00000023571 | −1.317554169 | 0.002212852 | 0.017761424 | 18.94630097 | 21.3669166 |
| ENSMUSG00000023800 | 1.312092825 | 2.45E−12 | 4.99E−10 | 459.697092 | 410.8275327 |
| ENSMUSG00000023905 | −1.475845141 | 9.44E−06 | 0.000275028 | 54.84455544 | 95.1799012 |
| ENSMUSG00000023927 | −1.218211099 | 0.003077602 | 0.022366209 | 27.92086459 | 26.223034 |
| ENSMUSG00000023968 | −1.267291327 | 0.008148531 | 0.04468095 | 10.96891109 | 11.65468178 |
| ENSMUSG00000024118 | −1.757053931 | 5.31E−18 | 2.79E−15 | 442.7451385 | 592.4463238 |
| ENSMUSG00000024130 | 1.042830636 | 1.30E−05 | 0.000354612 | 2989.526858 | 2745.648783 |
| ENSMUSG00000024136 | −1.225506878 | 0.002002269 | 0.016518413 | 27.92086459 | 19.42446963 |
| ENSMUSG00000024190 | −1.579039564 | 2.17E−13 | 5.48E−11 | 281.2029934 | 502.12254 |
| ENSMUSG00000024236 | 1.389584652 | 9.49E−13 | 2.12E−10 | 1344.190195 | 1041.151572 |
| ENSMUSG00000024411 | 1.68012077 | 5.07E−05 | 0.001068968 | 63.81911906 | 48.56117408 |
| ENSMUSG00000024440 | 1.007637941 | 0.000827422 | 0.008613399 | 107.6947634 | 103.9209125 |
| ENSMUSG00000024665 | 1.18951247 | 3.11E−12 | 6.05E−10 | 9244.7977 | 8635.147975 |
| ENSMUSG00000024843 | −1.175648724 | 2.45E−11 | 3.79E−09 | 1421.969747 | 1016.870985 |
| ENSMUSG00000024887 | 1.067323547 | 1.55E−06 | 6.39E−05 | 398.8694941 | 436.0793433 |
| ENSMUSG00000024924 | 1.265415535 | 2.60E−12 | 5.20E−10 | 642.1798855 | 816.7989481 |
| ENSMUSG00000024970 | −1.174479585 | 0.000322102 | 0.004379373 | 31.90955953 | 39.82016275 |
| ENSMUSG00000024978 | 2.527860907 | 4.51E−08 | 3.23E−06 | 10405.50793 | 5475.757989 |
| ENSMUSG00000025003 | 1.045766059 | 5.74E−07 | 2.75E−05 | 301.1464681 | 286.5109271 |
| ENSMUSG00000025006 | 1.229102652 | 2.44E−06 | 9.41E−05 | 288.1832095 | 294.2807149 |
| ENSMUSG00000025153 | 1.113433094 | 9.44E−07 | 4.18E−05 | 85604.37365 | 41744.15646 |
| ENSMUSG00000025161 | −1.470478397 | 0.001962044 | 0.016285081 | 18.94630097 | 9.712234816 |
| ENSMUSG00000025240 | 1.333245056 | 1.72E−06 | 6.99E−05 | 553.4314231 | 682.7701076 |
| ENSMUSG00000025323 | 1.252220218 | 7.14E−07 | 3.28E−05 | 138.6071492 | 127.2302761 |
| ENSMUSG00000025402 | −1.340941683 | 6.59E−15 | 2.25E−12 | 217.3838743 | 245.7195409 |
| ENSMUSG00000025429 | 1.693186246 | 2.17E−13 | 5.48E−11 | 624.2307583 | 540.0002558 |
| ENSMUSG00000025450 | −1.257708301 | 2.83E−05 | 0.000669372 | 30.91238579 | 62.15830282 |
| ENSMUSG00000025997 | −1.284615532 | 0.009041141 | 0.048049118 | 26.92369085 | 9.712234816 |
| ENSMUSG00000026020 | −1.156643623 | 9.59E−08 | 6.11E−06 | 250.2906076 | 294.2807149 |
| ENSMUSG00000026249 | −1.326755053 | 0.000451208 | 0.005606296 | 36.89542821 | 50.50362104 |
| ENSMUSG00000026358 | −2.793399328 | 5.66E−13 | 1.33E−10 | 12.96325856 | 11.65468178 |
| ENSMUSG00000026398 | 1.137826301 | 6.48E−12 | 1.16E−09 | 1403.023446 | 1667.590718 |
| ENSMUSG00000026471 | 1.000583106 | 1.46E−05 | 0.000384933 | 269.2369085 | 237.949753 |
| ENSMUSG00000026475 | −1.56210025 | 0.004964709 | 0.031300593 | 735.9142167 | 388.4893927 |
| ENSMUSG00000026525 | −1.790734538 | 0.001947167 | 0.0161813 | 7.977389882 | 8.741011335 |
| ENSMUSG00000026822 | −1.02651459 | 5.14E−06 | 0.00016724 | 122.6523694 | 119.4604882 |
| ENSMUSG00000026826 | −1.483020728 | 0.008821676 | 0.047306802 | 15.95477976 | 10.6834583 |
| ENSMUSG00000026832 | −1.174113684 | 0.001522033 | 0.013425591 | 30.91238579 | 34.96404534 |
| ENSMUSG00000027360 | −1.979964656 | 1.70E−12 | 3.69E−10 | 54.84455544 | 93.23745424 |
| ENSMUSG00000027398 | −1.057691022 | 0.001937845 | 0.016123489 | 78.77672509 | 37.87771578 |
| ENSMUSG00000027405 | −1.241634744 | 7.40E−06 | 0.000226395 | 225.3612642 | 254.4605522 |
| ENSMUSG00000027496 | −1.007831872 | 0.004782077 | 0.030501955 | 38.88977568 | 37.87771578 |
| ENSMUSG00000027513 | −1.115800194 | 1.04E−10 | 1.46E−08 | 16805.36896 | 18645.5484 |
| ENSMUSG00000027605 | 1.86697993 | 9.67E−16 | 3.77E−13 | 27327.54622 | 18714.50527 |
| ENSMUSG00000027762 | 1.17286147 | 2.98E−15 | 1.13E−12 | 1319.260852 | 1520.935972 |
| ENSMUSG00000027907 | −1.140239221 | 0.000303295 | 0.004203282 | 47.86433929 | 56.33096193 |
| ENSMUSG00000027947 | −1.05537492 | 2.15E−06 | 8.45E−05 | 434.7677486 | 468.1297181 |
| ENSMUSG00000028008 | 1.245889 | 0.000553467 | 0.00652119 | 54.84455544 | 81.58277246 |
| ENSMUSG00000028339 | 1.011959422 | 2.14E−08 | 1.71E−06 | 706.9961783 | 505.0362104 |
| ENSMUSG00000028445 | −1.767301879 | 0.003925136 | 0.026446185 | 70.79933521 | 71.87053764 |
| ENSMUSG00000028630 | 1.331166045 | 1.46E−07 | 8.59E−06 | 373.9401507 | 358.3814647 |
| ENSMUSG00000028838 | 1.693729472 | 3.36E−09 | 3.22E−07 | 304.1379893 | 151.5108631 |
| ENSMUSG00000028859 | −1.001034699 | 0.004539011 | 0.029294129 | 74.78803015 | 57.30218542 |
| ENSMUSG00000028862 | −2.278242573 | 1.62E−07 | 9.45E−06 | 8.974563618 | 9.712234816 |
| ENSMUSG00000028864 | 1.08474786 | 0.000792949 | 0.008337279 | 131.6269331 | 134.0288405 |
| ENSMUSG00000028957 | 2.966674755 | 1.69E−27 | 3.84E−24 | 314.1097266 | 609.9283465 |
| ENSMUSG00000028976 | 1.532387058 | 3.09E−07 | 1.58E−05 | 157.5534502 | 94.20867772 |
| ENSMUSG00000029086 | 1.631659452 | 1.01E−18 | 6.00E−16 | 713.9763945 | 617.6981343 |
| ENSMUSG00000029135 | −1.266157753 | 3.55E−07 | 1.81E−05 | 179.4912724 | 127.2302761 |
| ENSMUSG00000029188 | −2.354954255 | 1.89E−12 | 4.02E−10 | 42.87847062 | 12.62590526 |
| ENSMUSG00000029195 | 1.272024305 | 6.31E−09 | 5.74E−07 | 705.0018309 | 838.1658646 |
| ENSMUSG00000029370 | 1.683633222 | 1.05E−19 | 7.55E−17 | 546.4512069 | 472.9858356 |
| ENSMUSG00000029373 | −1.650047987 | 0.002725238 | 0.020577678 | 7.977389882 | 5.82734089 |
| ENSMUSG00000029380 | −2.803403219 | 8.38E−26 | 1.43E−22 | 53.84738171 | 128.2014996 |
| ENSMUSG00000029580 | −1.162469269 | 9.11E−12 | 1.55E−09 | 8321.414821 | 7155.974613 |
| ENSMUSG00000029591 | −1.593385799 | 3.01E−08 | 2.29E−06 | 43.87564435 | 60.21585586 |
| ENSMUSG00000029656 | −1.019716092 | 0.000134204 | 0.002299171 | 189.4630097 | 409.8563092 |
| ENSMUSG00000030032 | −1.061169015 | 0.007631272 | 0.042668167 | 23.93216965 | 14.56835222 |
| ENSMUSG00000030055 | 1.305889925 | 4.54E−06 | 0.000151075 | 239.3216965 | 263.2015635 |
| ENSMUSG00000030691 | 1.035229834 | 1.01E−08 | 8.79E−07 | 365.9627609 | 443.8491311 |
| ENSMUSG00000030782 | −1.087204324 | 0.00053618 | 0.006369234 | 38.88977568 | 39.82016275 |
| ENSMUSG00000030814 | −1.357189589 | 8.78E−10 | 9.81E−08 | 261.2595186 | 283.5972566 |
| ENSMUSG00000030827 | −1.631143551 | 1.45E−05 | 0.000383464 | 26.92369085 | 20.39569311 |
| ENSMUSG00000030934 | 1.434845811 | 0.000702548 | 0.007652274 | 13671.25191 | 24780.76713 |
| ENSMUSG00000030968 | −1.140744433 | 0.000133867 | 0.002296287 | 34.90108074 | 72.84176112 |
| ENSMUSG00000031010 | 1.281779593 | 3.26E−07 | 1.66E−05 | 953.2980909 | 1398.561814 |
| ENSMUSG00000031271 | −1.719181951 | 2.91E−23 | 3.97E−20 | 339.03907 | 470.0721651 |
| ENSMUSG00000031378 | 1.154152634 | 6.32E−15 | 2.21E−12 | 1014.125689 | 912.9500727 |
| ENSMUSG00000031465 | −1.254345174 | 0.0029265 | 0.021654196 | 21.93782218 | 11.65468178 |
| ENSMUSG00000031762 | −4.701992063 | 1.08E−05 | 0.000305681 | 384.9090618 | 188.4173554 |
| ENSMUSG00000031765 | −4.008275951 | 6.98E−08 | 4.64E−06 | 1129.797842 | 691.5111189 |
| ENSMUSG00000032009 | 1.096135097 | 4.98E−08 | 3.50E−06 | 816.6852892 | 873.12991 |
| ENSMUSG00000032064 | 1.076164714 | 3.75E−10 | 4.69E−08 | 659.131839 | 690.5398954 |
| ENSMUSG00000032083 | −1.056710751 | 2.91E−09 | 2.86E−07 | 57416.2665 | 56826.28591 |
| ENSMUSG00000032091 | 2.476316256 | 2.69E−06 | 0.000101175 | 102.7088947 | 50.50362104 |
| ENSMUSG00000032285 | 1.014640865 | 0.008899206 | 0.047498424 | 51.85303424 | 70.89931416 |
| ENSMUSG00000032417 | 1.412452049 | 0.000231229 | 0.003394264 | 102.7088947 | 50.50362104 |
| ENSMUSG00000032418 | 1.488424738 | 1.96E−09 | 2.00E−07 | 17569.20404 | 7712.485668 |
| ENSMUSG00000032500 | 2.09734091 | 1.65E−20 | 1.25E−17 | 413.8271001 | 367.1224761 |
| ENSMUSG00000032561 | 1.808480512 | 0.000942567 | 0.009458265 | 1361.142149 | 660.4319675 |
| ENSMUSG00000032702 | 1.072973429 | 8.45E−08 | 5.48E−06 | 1328.235415 | 1308.23803 |
| ENSMUSG00000032724 | 1.34458697 | 3.25E−10 | 4.18E−08 | 647.1657542 | 540.0002558 |
| ENSMUSG00000032735 | −1.26252931 | 1.21E−08 | 1.04E−06 | 191.4573572 | 257.3742226 |
| ENSMUSG00000032786 | −1.26794906 | 3.89E−08 | 2.81E−06 | 1778.957944 | 2029.857077 |
| ENSMUSG00000032849 | 1.170479352 | 7.00E−10 | 8.09E−08 | 403.8553628 | 338.9569951 |
| ENSMUSG00000032860 | 1.046181292 | 6.20E−08 | 4.19E−06 | 339.03907 | 263.2015635 |
| ENSMUSG00000032883 | 1.139189345 | 1.88E−11 | 3.09E−09 | 1754.0286 | 1475.288469 |
| ENSMUSG00000033105 | 1.210153299 | 1.43E−10 | 1.97E−08 | 3593.814142 | 2741.763889 |
| ENSMUSG00000033594 | −1.045179866 | 5.30E−08 | 3.67E−06 | 379.9231931 | 342.841889 |
| ENSMUSG00000033624 | 1.545958738 | 3.43E−10 | 4.33E−08 | 290.177557 | 397.230404 |
| ENSMUSG00000033792 | 1.183185841 | 0.000561984 | 0.006589659 | 63.81911906 | 67.98564371 |
| ENSMUSG00000033855 | 1.291796081 | 0.000477464 | 0.005865923 | 117.6665008 | 69.92809068 |
| ENSMUSG00000033967 | −2.822222129 | 4.36E−05 | 0.000952114 | 4.985868677 | 3.884893927 |
| ENSMUSG00000034066 | 1.2566902 | 2.38E−07 | 1.29E−05 | 275.2199509 | 346.7267829 |
| ENSMUSG00000034110 | 1.131917427 | 3.20E−05 | 0.000736514 | 99.71737353 | 110.7194769 |
| ENSMUSG00000034271 | −1.155793079 | 0.004733656 | 0.030334993 | 36.89542821 | 29.13670445 |
| ENSMUSG00000034755 | −1.51005032 | 0.006510973 | 0.037993212 | 7.977389882 | 5.82734089 |
| ENSMUSG00000034765 | −2.457798355 | 3.78E−06 | 0.000131464 | 23.93216965 | 12.62590526 |
| ENSMUSG00000034853 | 1.437062013 | 9.64E−14 | 2.63E−11 | 450.7225284 | 335.0721012 |
| ENSMUSG00000034926 | 1.093495822 | 1.31E−06 | 5.56E−05 | 17902.26007 | 19066.08817 |
| ENSMUSG00000035078 | 1.209517873 | 6.16E−05 | 0.001249206 | 322.0871165 | 384.6044987 |
| ENSMUSG00000035112 | −1.146398835 | 0.004042609 | 0.02701081 | 23.93216965 | 14.56835222 |
| ENSMUSG00000035164 | 1.06213892 | 8.60E−06 | 0.000256206 | 131.6269331 | 166.0792154 |
| ENSMUSG00000035165 | −1.798528938 | 1.18E−06 | 5.05E−05 | 32.90673326 | 22.33814008 |
| ENSMUSG00000035284 | 1.432291641 | 4.22E−09 | 3.95E−07 | 725.9424793 | 940.1443302 |
| ENSMUSG00000035900 | 1.041020638 | 2.29E−08 | 1.81E−06 | 343.0277649 | 318.561302 |
| ENSMUSG00000035933 | 1.189062904 | 7.63E−07 | 3.47E−05 | 188.465836 | 221.4389538 |
| ENSMUSG00000035948 | 1.741049509 | 1.78E−10 | 2.38E−08 | 816.6852892 | 893.5256031 |
| ENSMUSG00000036062 | −1.708320866 | 0.004715534 | 0.030261525 | 7.977389882 | 9.712234816 |
| ENSMUSG00000036120 | −1.340248384 | 3.85E−09 | 3.64E−07 | 263.2538661 | 219.4965068 |
| ENSMUSG00000036611 | 1.04665245 | 4.08E−07 | 2.03E−05 | 846.6005013 | 616.7269108 |
| ENSMUSG00000037035 | −1.785829601 | 2.14E−10 | 2.84E−08 | 59.83042412 | 30.10792793 |
| ENSMUSG00000037071 | 1.600161226 | 3.89E−11 | 5.71E−09 | 372268.8876 | 280319.3774 |
| ENSMUSG00000037095 | −1.027049761 | 7.90E−10 | 8.97E−08 | 3492.102421 | 3177.843232 |
| ENSMUSG00000037157 | 1.742314913 | 8.42E−06 | 0.000251859 | 51.85303424 | 60.21585586 |
| ENSMUSG00000037336 | −1.65964734 | 0.000936801 | 0.009424872 | 14.95760603 | 7.769787853 |
| ENSMUSG00000037443 | −1.349128808 | 2.89E−12 | 5.72E−10 | 750.8718227 | 1078.058065 |
| ENSMUSG00000037447 | −1.380120078 | 0.000135203 | 0.002310483 | 30.91238579 | 23.30936356 |
| ENSMIJSG00000037465 | −1.250556349 | 2.44E−12 | 4.99E−10 | 342.0305912 | 272.9137983 |
| ENSMUSG00000037583 | −1.178511493 | 2.71E−06 | 0.000101645 | 181.4856198 | 270.0001279 |
| ENSMUSG00000037709 | 1.621036788 | 2.49E−19 | 1.62E−16 | 850.5891962 | 1019.784656 |
| ENSMUSG00000037887 | −1.887921138 | 0.000434734 | 0.005464027 | 15.95477976 | 8.741011335 |
| ENSMUSG00000038217 | 1.023783748 | 9.74E−10 | 1.07E−07 | 3279.704415 | 3214.749724 |
| ENSMUSG00000038233 | 1.132248981 | 2.75E−05 | 0.000651686 | 765.8294287 | 340.8994421 |
| ENSMUSG00000038253 | −2.17957789 | 5.49E−08 | 3.76E−06 | 19.94347471 | 18.45324615 |
| ENSMUSG00000038370 | −1.863884427 | 6.22E−10 | 7.32E−08 | 95.72867859 | 197.1583668 |
| ENSMUSG00000038415 | −1.598903332 | 5.74E−10 | 6.80E−08 | 144.5901916 | 136.9425109 |
| ENSMUSG00000038418 | −2.528561862 | 1.50E−17 | 7.58E−15 | 95.72867859 | 79.64032549 |
| ENSMUSG00000038473 | 1.076370271 | 0.000625033 | 0.007061263 | 117.6665008 | 63.12952631 |
| ENSMUSG00000038530 | −1.722746304 | 4.58E−05 | 0.00098969 | 17.94912724 | 16.51079919 |
| ENSMUSG00000038583 | −1.702074711 | 0.00088397 | 0.009002758 | 12.96325856 | 13.59712874 |
| ENSMUSG00000038587 | −1.31455808 | 3.12E−06 | 0.000113043 | 97.72302606 | 73.8129846 |
| ENSMUSG00000038751 | 1.774904606 | 6.46E−12 | 1.16E−09 | 167.5251875 | 125.2878291 |
| ENSMUSG00000038768 | 1.297337324 | 1.64E−05 | 0.000422782 | 329.0673326 | 229.2087417 |
| ENSMUSG00000038774 | 1.007283954 | 3.14E−06 | 0.00011311 | 450.7225284 | 497.2664226 |
| ENSMUSG00000038844 | 1.003328537 | 1.78E−06 | 7.13E−05 | 364.9655871 | 380.7196048 |
| ENSMUSG00000038895 | −1.113485639 | 6.66E−08 | 4.45E−06 | 100.7145473 | 103.9209125 |
| ENSMUSG00000039103 | −1.109845105 | 0.002277587 | 0.018174053 | 37.89260194 | 21.3669166 |
| ENSMUSG00000039304 | 1.09876453 | 1.10E−06 | 4.76E−05 | 197.4403996 | 176.7626737 |
| ENSMUSG00000039533 | 1.597388363 | 4.59E−08 | 3.26E−06 | 367.9571083 | 378.7771578 |
| ENSMUSG00000039601 | 1.185885146 | 6.77E−14 | 1.97E−11 | 707.9933521 | 644.8923918 |
| ENSMUSG00000039704 | 1.942291849 | 0.00175377 | 0.014994461 | 267.2425611 | 391.4030631 |
| ENSMUSG00000039741 | 1.087450189 | 5.12E−08 | 3.58E−06 | 264.2510399 | 270.0001279 |
| ENSMUSG00000039853 | 1.374596414 | 3.52E−09 | 3.36E−07 | 477.6462192 | 438.0217902 |
| ENSMUSG00000039981 | 1.024216498 | 5.34E−05 | 0.001113305 | 116.669327 | 128.2014996 |
| ENSMUSG00000040093 | 1.487860932 | 2.33E−07 | 1.27E−05 | 234.3358278 | 188.4173554 |
| ENSMUSG00000040128 | −1.050165381 | 1.95E−11 | 3.16E−09 | 1378.094102 | 1310.180477 |
| ENSMUSG00000040152 | −1.254644006 | 0.001007039 | 0.009915514 | 35.89825447 | 19.42446963 |
| ENSMUSG00000040435 | −1.205190782 | 4.71E−06 | 0.000155637 | 119.6608482 | 131.11517 |
| ENSMUSG00000040584 | 1.185136822 | 2.65E−06 | 9.97E−05 | 302.1436418 | 375.8634874 |
| ENSMUSG00000040855 | 1.071792937 | 4.60E−07 | 2.25E−05 | 584.3438089 | 647.8060622 |
| ENSMUSG00000040891 | −2.088765162 | 3.05E−41 | 2.08E−37 | 406.846884 | 431.2232258 |
| ENSMUSG00000041134 | 1.050155799 | 0.006573499 | 0.03826156 | 41.88129688 | 57.30218542 |
| ENSMUSG00000041372 | 2.023614953 | 1.33E−05 | 0.000359411 | 51.85303424 | 49.53239756 |
| ENSMUSG00000041695 | −1.532466883 | 9.09E−05 | 0.001683702 | 33.903907 | 18.45324615 |
| ENSMUSG00000041702 | 1.159708962 | 1.79E−05 | 0.000451095 | 125.6438906 | 178.7051206 |
| ENSMUSG00000041920 | −1.612549242 | 5.78E−11 | 8.29E−09 | 130.6297593 | 116.5468178 |
| ENSMUSG00000041930 | −2.069670842 | 7.78E−10 | 8.92E−08 | 29.91521206 | 25.25181052 |
| ENSMUSG00000041945 | 2.034436558 | 0.000645414 | 0.007184911 | 35.89825447 | 23.30936356 |
| ENSMUSG00000042010 | 1.335313237 | 1.72E−08 | 1.42E−06 | 9652.641758 | 5265.973717 |
| ENSMUSG00000042115 | −1.249312452 | 6.34E−07 | 3.01E−05 | 57.83607665 | 47.5899506 |
| ENSMUSG00000042246 | −1.458803387 | 0.000333225 | 0.004503661 | 16.9519535 | 19.42446963 |
| ENSMUSG00000042333 | 1.17659167 | 0.000212792 | 0.003210009 | 83.76259377 | 76.72665505 |
| ENSMUSG00000042354 | −1.004338102 | 0.000195353 | 0.003017019 | 312.1153791 | 284.5684801 |
| ENSMUSG00000042379 | −1.128744357 | 0.001182685 | 0.011115132 | 63.81911906 | 39.82016275 |
| ENSMUSG00000042444 | 1.165704626 | 4.84E−10 | 5.85E−08 | 592.3211988 | 520.5757862 |
| ENSMUSG00000042510 | −1.154445577 | 0.002024484 | 0.016651324 | 28.91803832 | 29.13670445 |
| ENSMUSG00000042607 | −1.584091426 | 0.001074406 | 0.010435667 | 12.96325856 | 6.798564371 |
| ENSMUSG00000042622 | −1.343729967 | 0.000106867 | 0.001912519 | 29.91521206 | 22.33814008 |
| ENSMUSG00000042680 | 1.086900564 | 0.000262276 | 0.00374505 | 262.2566924 | 328.2735368 |
| ENSMUSG00000042743 | 1.143476031 | 0.005459928 | 0.033421057 | 39.88694941 | 35.93526882 |
| ENSMUSG00000042745 | −1.243661764 | 2.94E−08 | 2.25E−06 | 425.793185 | 215.6116129 |
| ENSMUSG00000043165 | −1.65740279 | 0.002445867 | 0.019147761 | 6.980216147 | 13.59712874 |
| ENSMUSG00000043421 | −1.515224307 | 3.07E−05 | 0.000711553 | 31.90955953 | 28.16548097 |
| ENSMUSG00000043639 | 1.042566076 | 0.00209562 | 0.017092091 | 83.76259377 | 70.89931416 |
| ENSMUSG00000043681 | −1.124534949 | 2.43E−09 | 2.42E−07 | 201.4290945 | 238.9209765 |
| ENSMUSG00000044042 | 1.154056163 | 3.06E−05 | 0.000711225 | 258.2679974 | 274.8562453 |
| ENSMUSG00000044186 | 1.148916053 | 0.009243418 | 0.048746497 | 35.89825447 | 33.99282186 |
| ENSMUSG00000044339 | −1.202694639 | 0.000240284 | 0.0035008 | 44.87281809 | 27.19425749 |
| ENSMUSG00000044349 | 1.020247724 | 0.000864482 | 0.008883909 | 170.5167087 | 218.5252834 |
| ENSMUSG00000044359 | 1.419594491 | 2.01E−07 | 1.12E−05 | 221.3725692 | 303.9929497 |
| ENSMUSG00000044676 | −1.235532178 | 3.06E−06 | 0.000111153 | 173.5082299 | 206.8706016 |
| ENSMUSG00000044749 | 1.260739468 | 0.003720434 | 0.025546608 | 2581.682801 | 3424.533996 |
| ENSMUSG00000044948 | −1.380324526 | 9.28E−05 | 0.001711935 | 28.91803832 | 18.45324615 |
| ENSMUSG00000045045 | 1.563883127 | 9.68E−08 | 6.14E−06 | 106.6975897 | 117.5180413 |
| ENSMUSG00000045294 | 1.216661032 | 2.49E−17 | 1.21E−14 | 16820.32657 | 15644.46784 |
| ENSMUSG00000045348 | −1.535176629 | 0.002140436 | 0.017319005 | 17.94912724 | 8.741011335 |
| ENSMUSG00000045382 | −1.705364762 | 8.30E−05 | 0.001575106 | 17.94912724 | 15.53957571 |
| ENSMUSG00000045411 | −1.265704961 | 6.98E−06 | 0.000215157 | 217.3838743 | 180.6475676 |
| ENSMUSG00000045776 | 1.435732199 | 4.30E−15 | 1.59E−12 | 1029.083295 | 1166.439401 |
| ENSMUSG00000045875 | 1.205673082 | 0.000172777 | 0.002749319 | 91.73998365 | 131.11517 |
| ENSMUSG00000046541 | 1.523461987 | 5.84E−06 | 0.000185032 | 69.80216147 | 95.1799012 |
| ENSMUSG00000046721 | −1.016110295 | 0.006334733 | 0.037187581 | 17.94912724 | 26.223034 |
| ENSMUSG00000046908 | −1.354651164 | 0.005396039 | 0.033236578 | 15.95477976 | 8.741011335 |
| ENSMUSG00000047496 | 1.093888518 | 1.32E−05 | 0.00035939 | 433.7705749 | 520.5757862 |
| ENSMUSG00000047649 | −1.227032883 | 5.45E−06 | 0.000175137 | 72.79368268 | 67.01442023 |
| ENSMUSG00000047875 | 1.019701978 | 0.000156296 | 0.002567963 | 101.711721 | 104.892136 |
| ENSMUSG00000048191 | −1.584188448 | 0.000509157 | 0.006122909 | 10.96891109 | 18.45324615 |
| ENSMUSG00000048644 | −2.138371107 | 3.35E−05 | 0.00076585 | 5.983042412 | 8.741011335 |
| ENSMUSG00000048856 | −1.529839383 | 8.95E−23 | 8.72E−20 | 3855.073661 | 3458.526818 |
| ENSMUSG00000049044 | −1.523296713 | 7.27E−17 | 3.42E−14 | 783.7785559 | 1199.461 |
| ENSMUSG00000049313 | 1.020089742 | 3.99E−06 | 0.000136353 | 328.0701589 | 236.9785295 |
| ENSMUSG00000049580 | −1.860685334 | 2.05E−30 | 5.59E−27 | 548.4455544 | 407.9138623 |
| ENSMUSG00000049791 | 1.116973741 | 1.01E−06 | 4.43E−05 | 636.1968431 | 678.8852137 |
| ENSMUSG00000049950 | −1.164244145 | 0.007174451 | 0.040867998 | 18.94630097 | 37.87771578 |
| ENSMUSG00000050390 | 1.148547066 | 4.80E−15 | 1.72E−12 | 1574.537328 | 1571.439593 |
| ENSMUSG00000050503 | −1.747508686 | 0.001612805 | 0.014098606 | 6.980216147 | 9.712234816 |
| ENSMUSG00000050663 | 1.366962359 | 0.003061462 | 0.022321797 | 20.94064844 | 51.47484453 |
| ENSMUSG00000050737 | −1.262831751 | 0.008424555 | 0.045643887 | 26.92369085 | 13.59712874 |
| ENSMUSG00000050914 | −2.867341906 | 0.000724516 | 0.007829023 | 13.96043229 | 23.30936356 |
| ENSMUSG00000051149 | 1.736018564 | 0.001879809 | 0.015833824 | 31.90955953 | 33.02159838 |
| ENSMUSG00000051339 | 1.051356464 | 2.17E−16 | 9.53E−14 | 1923.548135 | 1976.439785 |
| ENSMUSG00000051452 | 1.412708477 | 1.55E−10 | 2.11E−08 | 276.2171247 | 240.8634234 |
| ENSMUSG00000051674 | 1.054513908 | 9.53E−09 | 8.39E−07 | 515.5388212 | 743.9571869 |
| ENSMUSG00000051998 | −1.5364341 | 0.002646663 | 0.020230437 | 9.971737353 | 19.42446963 |
| ENSMUSG00000052085 | 1.272820714 | 3.30E−11 | 4.99E−09 | 423.7988375 | 546.7988202 |
| ENSMUSG00000052595 | 1.233431784 | 0.003777269 | 0.025781088 | 1666.277312 | 2467.878867 |
| ENSMUSG00000052656 | 1.063492715 | 4.09E−12 | 7.63E−10 | 4427.451385 | 3448.814583 |
| ENSMUSG00000052684 | −1.26752533 | 1.09E−07 | 6.78E−06 | 161.5421451 | 161.223098 |
| ENSMUSG00000052713 | 1.631079765 | 2.29E−06 | 8.94E−05 | 74.78803015 | 84.4964429 |
| ENSMUSG00000052837 | −1.91589408 | 1.98E−18 | 1.08E−15 | 205.4177895 | 302.0505028 |
| ENSMUSG00000053560 | −1.145431158 | 4.41E−10 | 5.44E−08 | 324.081464 | 268.0576809 |
| ENSMUSG00000053964 | −1.860738134 | 2.78E−14 | 8.62E−12 | 100.7145473 | 119.4604882 |
| ENSMUSG00000053977 | −1.428502316 | 0.007562752 | 0.042337131 | 8.974563618 | 28.16548097 |
| ENSMUSG00000054008 | 1.359856014 | 0.005424595 | 0.033352211 | 3190.955953 | 3047.699285 |
| ENSMUSG00000054150 | 1.583267643 | 1.23E−07 | 7.44E−06 | 98.72019979 | 105.8633595 |
| ENSMUSG00000054422 | −1.006699851 | 5.17E−07 | 2.51E−05 | 21378.40771 | 26089.97639 |
| ENSMUSG00000054453 | 1.090734081 | 0.000462815 | 0.005711685 | 93.73433112 | 87.41011335 |
| ENSMUSG00000054659 | 1.244126007 | 0.002259781 | 0.018053098 | 79.77389882 | 73.8129846 |
| ENSMUSG00000054932 | 1.219377704 | 9.42E−05 | 0.001724207 | 87.75128871 | 202.0144842 |
| ENSMUSG00000055148 | −1.210391107 | 1.04E−07 | 6.56E−06 | 210.4036581 | 153.4533101 |
| ENSMUSG00000055254 | 1.702350158 | 1.36E−13 | 3.56E−11 | 1260.427601 | 1100.396205 |
| ENSMUSG00000055491 | −1.429157403 | 5.36E−08 | 3.69E−06 | 191.4573572 | 205.8993781 |
| ENSMUSG00000055660 | 1.21539125 | 9.47E−05 | 0.001725945 | 86.75411497 | 97.12234816 |
| ENSMUSG00000055692 | −1.507668302 | 6.91E−05 | 0.001367589 | 23.93216965 | 24.28058704 |
| ENSMUSG00000055980 | 1.007478258 | 8.74E−05 | 0.001638705 | 336.0475488 | 377.8059344 |
| ENSMUSG00000056054 | −4.00774487 | 1.39E−20 | 1.11E−17 | 6.980216147 | 6.798564371 |
| ENSMUSG00000056071 | −3.639131514 | 1.15E−24 | 1.74E−21 | 15.95477976 | 8.741011335 |
| ENSMUSG00000056091 | −1.031452251 | 1.62E−09 | 1.68E−07 | 1990.358776 | 1481.115809 |
| ENSMUSG00000056148 | 1.3596028 | 0.000119043 | 0.002094688 | 75.78520388 | 108.7770299 |
| ENSMUSG00000056313 | −1.726808914 | 2.85E−11 | 4.37E−09 | 435.7649223 | 244.7483174 |
| ENSMUSG00000057342 | 1.09304789 | 8.77E−15 | 2.92E−12 | 1269.402165 | 1384.964685 |
| ENSMUSG00000057604 | −1.272122676 | 0.008168023 | 0.044709526 | 10.96891109 | 10.6834583 |
| ENSMUSG00000057722 | −1.840565518 | 1.78E−07 | 1.02E−05 | 45.86999182 | 35.93526882 |
| ENSMUSG00000057969 | 1.066228289 | 0.000219235 | 0.003267437 | 95.72867859 | 74.78420809 |
| ENSMUSG00000058207 | −1.338541988 | 1.08E−13 | 2.88E−11 | 23432.58561 | 23413.28447 |
| ENSMUSG00000058503 | −1.136818503 | 6.67E−07 | 3.10E−05 | 130.6297593 | 164.1367684 |
| ENSMUSG00000058793 | 1.056167061 | 4.57E−10 | 5.57E−08 | 3494.096768 | 3071.008649 |
| ENSMUSG00000058794 | 1.61977926 | 2.23E−06 | 8.73E−05 | 144.5901916 | 102.9496891 |
| ENSMUSG00000058921 | 1.372756574 | 4.51E−07 | 2.22E−05 | 911.4167941 | 1126.619239 |
| ENSMUSG00000059149 | 1.084964814 | 0.00015449 | 0.002547498 | 343.0277649 | 296.2231619 |
| ENSMUSG00000059824 | 4.510710868 | 5.51E−13 | 1.32E−10 | 248.2962601 | 730.3600582 |
| ENSMUSG00000060429 | 1.717674566 | 1.22E−12 | 2.68E−10 | 734.9170429 | 831.3673003 |
| ENSMUSG00000061175 | 1.152852085 | 3.29E−14 | 9.98E−12 | 1100.879804 | 976.079599 |
| ENSMUSG00000061292 | 1.112980568 | 1.04E−05 | 0.000296719 | 1260.427601 | 1520.935972 |
| ENSMUSG00000061436 | 1.042732241 | 2.40E−05 | 0.000578522 | 326.0758114 | 431.2232258 |
| ENSMUSG00000061536 | 1.045389319 | 1.00E−05 | 0.000287463 | 219.3782218 | 260.2878931 |
| ENSMUSG00000061825 | 1.826944112 | 9.41E−14 | 2.63E−11 | 718.9622632 | 690.5398954 |
| ENSMUSG00000062901 | 1.319446488 | 6.22E−09 | 5.70E−07 | 788.7644246 | 893.5256031 |
| ENSMUSG00000063535 | 1.201850675 | 0.000151738 | 0.002508185 | 62.82194532 | 71.87053764 |
| ENSMUSG00000063704 | −1.127471271 | 2.47E−07 | 1.32E−05 | 294.1662519 | 311.7627376 |
| ENSMUSG00000063929 | −1.198836957 | 5.83E−06 | 0.000185032 | 50.8558605 | 105.8633595 |
| ENSMUSG00000065126 | −1.713055356 | 1.38E−07 | 8.18E−06 | 29.91521206 | 48.56117408 |
| ENSMUSG00000065147 | −1.294541943 | 0.007255788 | 0.041125179 | 14.95760603 | 12.62590526 |
| ENSMUSG00000065952 | 1.57267976 | 1.62E−10 | 2.19E−08 | 199.4347471 | 157.338204 |
| ENSMUSG00000066456 | 1.032022635 | 0.005405213 | 0.033278057 | 76.78237762 | 64.10074979 |
| ENSMUSG00000066477 | −1.128543552 | 2.29E−05 | 0.000561201 | 57.83607665 | 44.67628015 |
| ENSMUSG00000066687 | −2.591322343 | 5.98E−23 | 6.28E−20 | 139.6043229 | 122.3741587 |
| ENSMUSG00000066944 | 1.544707207 | 0.000412457 | 0.005261624 | 43.87564435 | 49.53239756 |
| ENSMUSG00000067149 | −1.843573184 | 0.000282252 | 0.003980428 | 113.6778058 | 169.9641093 |
| ENSMUSG00000068463 | −2.382320454 | 0.000472976 | 0.005821273 | 4.985868677 | 3.884893927 |
| ENSMUSG00000068742 | 1.190690012 | 3.19E−12 | 6.14E−10 | 611.2674997 | 770.1802209 |
| ENSMUSG00000068877 | −1.095647878 | 6.50E−07 | 3.04E−05 | 98.72019979 | 98.09357164 |
| ENSMUSG00000069456 | 1.727916701 | 1.48E−26 | 2.89E−23 | 6191.451722 | 5969.139518 |
| ENSMUSG00000069804 | −2.067148134 | 1.72E−05 | 0.000437213 | 3.988694941 | 13.59712874 |
| ENSMUSG00000070576 | 1.095801322 | 1.06E−07 | 6.63E−06 | 253.2821288 | 274.8562453 |
| ENSMUSG00000070583 | 1.188428844 | 0.006226465 | 0.036681868 | 41.88129688 | 47.5899506 |
| ENSMUSG00000071076 | −1.243260003 | 3.56E−08 | 2.62E−06 | 1324.24672 | 1340.288405 |
| ENSMUSG00000071456 | 1.345823726 | 0.001263422 | 0.011712633 | 45.86999182 | 70.89931416 |
| ENSMUSG00000071547 | −1.168738292 | 0.00025901 | 0.003706323 | 44.87281809 | 25.25181052 |
| ENSMUSG00000071637 | −1.028910519 | 0.000250761 | 0.0036106 | 107.6947634 | 64.10074979 |
| ENSMUSG00000071645 | −1.14015686 | 5.54E−07 | 2.68E−05 | 206.4149632 | 273.8850218 |
| ENSMUSG00000072294 | 2.084673623 | 3.53E−08 | 2.62E−06 | 97.72302606 | 148.5971927 |
| ENSMUSG00000072571 | 1.01455077 | 0.003907572 | 0.026379983 | 56.83890291 | 47.5899506 |
| ENSMUSG00000072664 | 1.275253331 | 1.57E−06 | 6.41E−05 | 951.3037435 | 1401.475484 |
| ENSMUSG00000072692 | −1.135811828 | 0.000365333 | 0.004818223 | 34.90108074 | 29.13670445 |
| ENSMUSG00000072849 | −2.106422019 | 7.14E−21 | 6.08E−18 | 100.7145473 | 96.15112468 |
| ENSMUSG00000072999 | −1.145851135 | 1.86E−07 | 1.05E−05 | 116.669327 | 176.7626737 |
| ENSMUSG00000073460 | −1.28577089 | 0.005211876 | 0.032439227 | 18.94630097 | 39.82016275 |
| ENSMUSG00000073835 | 2.295482419 | 0.000631649 | 0.007089542 | 332.0588539 | 185.503685 |
| ENSMUSG00000074024 | −1.08003527 | 4.28E−05 | 0.000939631 | 43.87564435 | 61.18707934 |
| ENSMUSG00000074063 | −2.210318446 | 1.23E−07 | 7.44E−06 | 989.1963454 | 819.7126185 |
| ENSMUSG00000074213 | 1.225081814 | 0.003513884 | 0.024460869 | 87.75128871 | 38.84893927 |
| ENSMUSG00000074345 | 1.610694413 | 1.86E−06 | 7.43E−05 | 92.73715738 | 95.1799012 |
| ENSMUSG00000074375 | 1.123374183 | 1.71E−16 | 7.76E−14 | 1739.070994 | 2059.965005 |
| ENSMUSG00000074876 | 1.013446872 | 0.000205306 | 0.003120937 | 101.711721 | 119.4604882 |
| ENSMUSG00000075470 | 1.495450545 | 0.008033121 | 0.044154642 | 346.0192861 | 387.5181692 |
| ENSMUSG00000075552 | 1.02486804 | 2.18E−07 | 1.20E−05 | 1632.373405 | 1558.813688 |
| ENSMUSG00000075590 | −1.36961476 | 1.11E−14 | 3.51E−12 | 556.4229443 | 571.0794072 |
| ENSMUSG00000076490 | −1.690670704 | 0.002848332 | 0.021272015 | 9.971737353 | 4.856117408 |
| ENSMUSG00000076569 | −1.340585852 | 0.000314538 | 0.004311858 | 26.92369085 | 70.89931416 |
| ENSMUSG00000076596 | −3.365323343 | 1.13E−07 | 6.96E−06 | 3.988694941 | 6.798564371 |
| ENSMUSG00000076609 | −2.421594655 | 3.22E−06 | 0.000115075 | 484.6264354 | 675.0003197 |
| ENSMUSG00000076613 | −1.082440472 | 0.000721125 | 0.007817157 | 31.90955953 | 61.18707934 |
| ENSMUSG00000076617 | −1.218011643 | 6.44E−08 | 4.32E−06 | 1755.025774 | 1885.144778 |
| ENSMUSG00000076934 | −3.037010136 | 1.44E−09 | 1.52E−07 | 7.977389882 | 2.913670445 |
| ENSMUSG00000077148 | 1.938039708 | 8.47E−06 | 0.000252598 | 48.86151303 | 38.84893927 |
| ENSMUSG00000078193 | −1.269506646 | 0.004582659 | 0.029506003 | 13.96043229 | 9.712234816 |
| ENSMUSG00000078234 | 1.031251449 | 6.47E−07 | 3.04E−05 | 557.420118 | 503.0937635 |
| ENSMUSG00000078650 | −1.017019231 | 3.99E−07 | 2.00E−05 | 5816.514398 | 3628.490927 |
| ENSMUSG00000078651 | −2.099102297 | 6.20E−07 | 2.95E−05 | 8.974563618 | 10.6834583 |
| ENSMUSG00000078672 | −1.051061583 | 1.66E−05 | 0.000428362 | 108.6919371 | 197.1583668 |
| ENSMUSG00000078688 | 1.787709931 | 3.76E−11 | 5.58E−09 | 119.6608482 | 143.7410753 |
| ENSMUSG00000078817 | −1.628755428 | 1.37E−11 | 2.31E−09 | 107.6947634 | 79.64032549 |
| ENSMUSG00000079017 | −1.001509959 | 0.000541038 | 0.006404635 | 74.78803015 | 60.21585586 |
| ENSMUSG00000079036 | −1.378588057 | 0.008915895 | 0.047568881 | 170.5167087 | 124.3166056 |
| ENSMUSG00000079065 | −1.153356662 | 4.39E−06 | 0.000146637 | 123.6495432 | 164.1367684 |
| ENSMUSG00000079465 | −1.030415111 | 0.006780187 | 0.03914539 | 18.94630097 | 27.19425749 |
| ENSMUSG00000079470 | 1.222302248 | 2.09E−11 | 3.33E−09 | 498.5868677 | 463.2736007 |
| ENSMUSG00000080059 | −1.546208951 | 0.008392591 | 0.045561211 | 7.977389882 | 9.712234816 |
| ENSMUSG00000081344 | −1.007584184 | 0.007335419 | 0.041406148 | 22.93499591 | 19.42446963 |
| ENSMUSG00000082065 | 2.441656125 | 0.003600075 | 0.02496215 | 179.4912724 | 141.7986283 |
| ENSMUSG00000082173 | 2.591199516 | 2.10E−11 | 3.33E−09 | 150.573234 | 114.6043708 |
| ENSMUSG00000082586 | 1.935822229 | 3.52E−06 | 0.000123572 | 53.84738171 | 55.35973845 |
| ENSMUSG00000082658 | −1.204694651 | 0.004700851 | 0.030195718 | 67.807814 | 23.30936356 |
| ENSMUSG00000083327 | 1.031096879 | 0.000937166 | 0.009424872 | 102.7088947 | 96.15112468 |
| ENSMUSG00000083621 | −1.163358375 | 0.003315291 | 0.023547198 | 19.94347471 | 19.42446963 |
| ENSMUSG00000083716 | −1.168157812 | 0.001666635 | 0.01443041 | 25.92651712 | 25.25181052 |
| ENSMUSG00000083813 | −1.588176086 | 8.35E−05 | 0.001580916 | 11.96608482 | 53.41729149 |
| ENSMUSG00000083863 | −1.215047324 | 0.000627696 | 0.007061263 | 24.92934338 | 19.42446963 |
| ENSMUSG00000083992 | −1.283026783 | 0.003457083 | 0.024176532 | 11.96608482 | 12.62590526 |
| ENSMUSG00000084822 | −1.954137616 | 1.52E−07 | 8.88E−06 | 16.9519535 | 25.25181052 |
| ENSMUSG00000084883 | 1.839966406 | 9.14E−09 | 8.09E−07 | 157.5534502 | 141.7986283 |
| ENSMUSG00000085001 | −2.14213554 | 3.42E−08 | 2.56E−06 | 29.91521206 | 16.51079919 |
| ENSMUSG00000085156 | −1.793249778 | 0.00014396 | 0.002408808 | 24.92934338 | 31.07915141 |
| ENSMUSG00000085445 | −1.469856228 | 8.55E−08 | 5.52E−06 | 45.86999182 | 46.61872712 |
| ENSMUSG00000085834 | −3.692490083 | 4.07E−39 | 1.85E−35 | 50.8558605 | 56.33096193 |
| ENSMUSG00000085995 | −1.230019641 | 9.11E−06 | 0.000268289 | 189.4630097 | 167.0504388 |
| ENSMUSG00000086140 | 1.216209638 | 0.003436989 | 0.024085413 | 34.90108074 | 60.21585586 |
| ENSMUSG00000086446 | −2.158985376 | 0.000113634 | 0.002012508 | 17.94912724 | 6.798564371 |
| ENSMUSG00000086529 | 2.155075918 | 4.95E−05 | 0.001053778 | 27.92086459 | 38.84893927 |
| ENSMUSG00000086786 | 1.909493382 | 0.004138057 | 0.027366965 | 31.90955953 | 14.56835222 |
| ENSMUSG00000086844 | −1.797314635 | 1.20E−05 | 0.000331862 | 10.96891109 | 17.48202267 |
| ENSMUSG00000087382 | −1.074896574 | 1.10E−05 | 0.000308734 | 161.5421451 | 285.5397036 |
| ENSMUSG00000087445 | −1.629934605 | 4.98E−05 | 0.001058506 | 21.93782218 | 12.62590526 |
| ENSMUSG00000087595 | −1.356715257 | 0.000782501 | 0.008272069 | 15.95477976 | 21.3669166 |
| ENSMUSG00000087613 | 1.300460925 | 0.002649524 | 0.020230437 | 53.84738171 | 60.21585586 |
| ENSMUSG00000087616 | −2.549514093 | 7.92E−06 | 0.000238545 | 14.95760603 | 13.59712874 |
| ENSMUSG00000087658 | −1.652423913 | 0.001338341 | 0.012240744 | 12.96325856 | 5.82734089 |
| ENSMUSG00000089726 | −1.506918387 | 4.30E−05 | 0.000942053 | 73.79085641 | 59.24463238 |
| ENSMUSG00000089943 | 1.766690487 | 7.03E−13 | 1.62E−10 | 3075.2838 | 2604.821378 |
| ENSMUSG00000090021 | −1.656392969 | 0.002988368 | 0.021957099 | 5.983042412 | 7.769787853 |
| ENSMUSG00000090145 | 1.252091278 | 1.05E−06 | 4.56E−05 | 514.5416474 | 669.1729788 |
| ENSMUSG00000090175 | 1.660039728 | 0.006419999 | 0.037574902 | 840.6174589 | 1098.453758 |
| ENSMUSG00000090264 | −2.18334921 | 1.38E−07 | 8.18E−06 | 7.977389882 | 12.62590526 |
| ENSMUSG00000090369 | 1.713925914 | 3.56E−08 | 2.62E−06 | 82.76542003 | 126.2590526 |
| ENSMUSG00000090555 | −2.901973235 | 9.10E−10 | 1.01E−07 | 121.6551957 | 110.7194769 |
| ENSMUSG00000090610 | 1.076721434 | 0.00270073 | 0.020438322 | 79.77389882 | 53.41729149 |
| ENSMUSG00000090698 | −2.55687976 | 1.21E−08 | 1.04E−06 | 20.94064844 | 19.42446963 |
| ENSMUSG00000091021 | −1.527245218 | 0.003008341 | 0.022044462 | 10.96891109 | 16.51079919 |
| ENSMUSG00000091509 | −1.002937779 | 0.000805178 | 0.008420412 | 69.80216147 | 64.10074979 |
| ENSMUSG00000092075 | −5.716673555 | 4.92E−16 | 1.97E−13 | 2.991521206 | 2.913670445 |
| ENSMUSG00000094410 | 1.220382244 | 0.001002234 | 0.009882478 | 53.84738171 | 132.0863935 |
| ENSMUSG00000095280 | −1.650205335 | 4.03E−05 | 0.000893784 | 58.83325038 | 25.25181052 |
| ENSMUSG00000095351 | −3.092804449 | 0.006028299 | 0.035789253 | 6.980216147 | 60.21585586 |
| ENSMUSG00000096833 | 3.208484861 | 0.004128622 | 0.02736411 | 10.96891109 | 124.3166056 |
| ENSMUSG00000096910 | 1.124675865 | 0.006036 | 0.035819378 | 64.81629279 | 67.01442023 |
| ENSMUSG00000096954 | 1.206343759 | 0.003120612 | 0.022529545 | 33.903907 | 87.41011335 |
| ENSMUSG00000097124 | 2.16107918 | 3.03E−13 | 7.50E−11 | 133.6212805 | 103.9209125 |
| ENSMUSG00000097221 | 1.550011559 | 0.000809985 | 0.008457711 | 46.86716556 | 44.67628015 |
| ENSMUSG00000097312 | −1.433041605 | 0.000236458 | 0.003456142 | 49.85868677 | 34.96404534 |
| ENSMUSG00000097536 | 1.179506676 | 0.003789911 | 0.025815692 | 36.89542821 | 55.35973845 |
| ENSMUSG00000097615 | −1.278009058 | 7.24E−05 | 0.001413729 | 33.903907 | 25.25181052 |
| ENSMUSG00000097660 | −2.932543755 | 3.83E−06 | 0.000131806 | 4.985868677 | 4.856117408 |
| ENSMUSG00000097691 | 1.274551418 | 4.53E−08 | 3.23E−06 | 235.3330015 | 259.3166696 |
| ENSMUSG00000097743 | −1.078551643 | 0.000185309 | 0.002900208 | 55.84172918 | 55.35973845 |
| ENSMUSG00000097908 | 1.156232879 | 5.54E−05 | 0.001144881 | 154.561929 | 95.1799012 |
| ENSMUSG00000097971 | −1.77227667 | 3.92E−13 | 9.56E−11 | 2017.282467 | 2103.670061 |
| ENSMUSG00000097994 | −1.156597047 | 0.008206265 | 0.04482173 | 14.95760603 | 25.25181052 |
| ENSMUSG00000098041 | −1.469996023 | 0.000332181 | 0.004498467 | 22.93499591 | 20.39569311 |
| ENSMUSG00000098661 | −1.627002493 | 0.000277688 | 0.003922434 | 19.94347471 | 6.798564371 |
| ENSMUSG00000098814 | −4.705165719 | 0.001128634 | 0.010794205 | 2.991521206 | 1.942446963 |
| ENSMUSG00000098882 | 1.905068974 | 0.000139968 | 0.002360908 | 128.6354119 | 27.19425749 |
| ENSMUSG00000099568 | −2.251364762 | 0.000320113 | 0.004365382 | 1.994347471 | 4.856117408 |
| ENSMUSG00000099858 | 1.088333588 | 4.80E−05 | 0.001024996 | 154.561929 | 202.0144842 |
| ENSMUSG00000100094 | 1.758512646 | 5.46E−23 | 6.28E−20 | 3175.001173 | 2305.684545 |
| ENSMUSG00000100468 | 1.995883006 | 0.000292618 | 0.004105376 | 17.94912724 | 36.9064923 |
| ENSMUSG00000101939 | −1.264629649 | 0.000784437 | 0.008279693 | 14.95760603 | 28.16548097 |
| ENSMUSG00000102275 | −1.950030022 | 0.000225327 | 0.003321924 | 4.985868677 | 13.59712874 |
| ENSMUSG00000102577 | −1.649520189 | 1.70E−08 | 1.42E−06 | 40.88412315 | 64.10074979 |
| ENSMUSG00000102719 | −1.730894623 | 0.006769906 | 0.039102589 | 12.96325856 | 5.82734089 |
| ENSMUSG00000102869 | 1.362067771 | 5.85E−06 | 0.000185032 | 292.1719044 | 335.0721012 |
| ENSMUSG00000102882 | 2.19227753 | 3.68E−07 | 1.87E−05 | 88.74846244 | 63.12952631 |
| ENSMUSG00000102918 | 1.26420677 | 0.002814898 | 0.021114835 | 30.91238579 | 47.5899506 |
| ENSMUSG00000103285 | −1.692405475 | 0.00269655 | 0.020438322 | 9.971737353 | 5.82734089 |
| ENSMUSG00000103546 | −1.97976669 | 0.000414043 | 0.005267075 | 6.980216147 | 6.798564371 |
| ENSMUSG00000104030 | −3.028455317 | 2.18E−07 | 1.20E−05 | 3.988694941 | 4.856117408 |
| ENSMUSG00000104388 | −2.61061807 | 5.15E−05 | 0.001078975 | 6.980216147 | 1.942446963 |
| ENSMUSG00000104399 | −1.177892321 | 0.00272173 | 0.020574411 | 23.93216965 | 29.13670445 |
| ENSMUSG00000104445 | 1.012908858 | 0.003001159 | 0.022015494 | 68.80498774 | 51.47484453 |
| ENSMUSG00000104973 | −2.007621104 | 9.36E−05 | 0.001719883 | 6.980216147 | 13.59712874 |
| ENSMUSG00000105161 | −1.279752452 | 0.00691512 | 0.039789659 | 21.93782218 | 8.741011335 |
| ENSMUSG00000105434 | −2.02241612 | 0.000742647 | 0.007975668 | 5.983042412 | 7.769787853 |
| ENSMUSG00000105547 | −2.707072316 | 4.65E−06 | 0.000153843 | 5.983042412 | 2.913670445 |
| ENSMUSG00000105556 | 1.504518371 | 0.00309422 | 0.022420764 | 21.93782218 | 29.13670445 |
| ENSMUSG00000105703 | −1.801332207 | 3.38E−12 | 6.40E−10 | 242.3132177 | 122.3741587 |
| ENSMUSG00000105881 | 1.259909682 | 7.19E−05 | 0.001410701 | 247.2990864 | 245.7195409 |
| ENSMUSG00000105906 | −2.671616891 | 0.003078263 | 0.022366209 | 6.980216147 | 20.39569311 |
| ENSMUSG00000106030 | 1.145150269 | 5.54E−05 | 0.001144881 | 109.6891109 | 147.6259692 |
| ENSMUSG00000106664 | −1.606191029 | 0.000152255 | 0.002513686 | 9.971737353 | 17.48202267 |
| ENSMUSG00000106705 | −2.05446715 | 2.41E−09 | 2.41E−07 | 18.94630097 | 32.05037489 |
| ENSMUSG00000106706 | −1.806072384 | 0.002749571 | 0.020715964 | 9.971737353 | 8.741011335 |
| ENSMUSG00000106943 | −1.230168101 | 0.009404129 | 0.049324655 | 9.971737353 | 13.59712874 |
| ENSMUSG00000107168 | −2.535260536 | 5.76E−05 | 0.00118059 | 1.994347471 | 9.712234816 |
| ENSMUSG00000107225 | −3.314781204 | 7.31E−09 | 6.61E−07 | 3.988694941 | 1.942446963 |
| ENSMUSG00000107304 | −1.772473058 | 0.001145745 | 0.010886341 | 7.977389882 | 10.6834583 |
| ENSMUSG00000107390 | −1.580957843 | 0.000625578 | 0.007061263 | 12.96325856 | 23.30936356 |
| ENSMUSG00000107624 | −2.865902972 | 2.57E−10 | 3.34E−08 | 8.974563618 | 13.59712874 |
| ENSMUSG00000108368 | −1.782724603 | 5.57E−05 | 0.001147878 | 20.94064844 | 11.65468178 |
| ENSMUSG00000108633 | −1.682864395 | 0.001365295 | 0.012400579 | 8.974563618 | 12.62590526 |
| ENSMUSG00000108820 | −1.905612833 | 0.000888445 | 0.00904159 | 6.980216147 | 3.884893927 |
| ENSMUSG00000108825 | 1.256782178 | 0.000111922 | 0.001987343 | 104.7032422 | 117.5180413 |
| ENSMUSG00000109089 | −1.779914445 | 1.06E−06 | 4.61E−05 | 42.87847062 | 34.96404534 |
| ENSMUSG00000109115 | 2.077699358 | 0.000502824 | 0.006083704 | 26.92369085 | 27.19425749 |
| ENSMUSG00000109157 | −1.885864825 | 0.000870552 | 0.00892677 | 4.985868677 | 6.798564371 |
| ENSMUSG00000109262 | 1.329001341 | 0.002840836 | 0.0212393 | 48.86151303 | 45.64750364 |
| ENSMUSG00000109291 | −1.949878064 | 0.001232984 | 0.01148511 | 1.994347471 | 9.712234816 |
| ENSMUSG00000109536 | −1.039899156 | 0.001827878 | 0.015463254 | 63.81911906 | 37.87771578 |
| ENSMUSG00000109555 | −1.255499819 | 0.007385438 | 0.041635066 | 10.96891109 | 14.56835222 |
| ENSMUSG00000109807 | −2.757236067 | 2.47E−09 | 2.44E−07 | 11.96608482 | 5.82734089 |
| ENSMUSG00000109836 | 1.535086391 | 0.000730915 | 0.007885666 | 46.86716556 | 68.9568672 |
| ENSMUSG00000109841 | 1.421032277 | 0.000627493 | 0.007061263 | 46.86716556 | 57.30218542 |
| ENSMUSG00000110588 | −5.501119532 | 2.73E−06 | 0.000102051 | 31.90955953 | 14.56835222 |
| ENSMUSG00000110613 | 1.168711997 | 0.002147567 | 0.017360028 | 64.81629279 | 58.2734089 |
| ENSMUSG00000110702 | −1.389252147 | 0.004080864 | 0.027194141 | 18.94630097 | 11.65468178 |
| ENSMUSG00000110755 | 1.852337964 | 4.42E−06 | 0.000147317 | 194.4488784 | 92.26623075 |
| ENSMUSG00000111282 | −1.321989153 | 0.002140143 | 0.017319005 | 21.93782218 | 18.45324615 |
| ENSMUSG00000111312 | 1.254957582 | 0.001977069 | 0.016387107 | 45.86999182 | 33.99282186 |
| ENSMUSG00000111631 | −1.948317608 | 1.41E−05 | 0.000376102 | 11.96608482 | 15.53957571 |
| ENSMUSG00000111709 | 2.018073354 | 0.00211263 | 0.017179449 | 31.90955953 | 69.92809068 |
| ENSMUSG00000111774 | 2.335615682 | 0.000501244 | 0.006070575 | 17.94912724 | 15.53957571 |

| ID | agedliver92183tox | agedliverpbs1 | agedliverpbs2 | agedliverpbs3 | Gene.name |
|---|---|---|---|---|---|
| ENSMUSG00000000204 | 11.93770314 | 61.79909158 | 46.97322185 | 22.5130033 | Slfn4 |
| ENSMUSG00000000317 | 55.0970914 | 92.69863737 | 140.9196656 | 205.5535084 | Bcl6b |
| ENSMUSG00000000686 | 469.2435618 | 171.5457542 | 161.796653 | 294.6266953 | Abhd15 |
| ENSMUSG00000001227 | 92.74677053 | 171.5457542 | 203.550628 | 184.9981575 | Sema6b |
| ENSMUSG00000001403 | 11.01941828 | 51.14407579 | 37.57857748 | 17.61887214 | Ube2c |
| ENSMUSG00000001983 | 299.3608633 | 128.9256911 | 122.1303768 | 219.2570756 | Taco1 |
| ENSMUSG00000002233 | 141.4158679 | 330.3054895 | 288.1024274 | 298.5420002 | Rhoc |
| ENSMUSG00000002250 | 415.9830401 | 627.58043 | 657.6251059 | 647.9829644 | Ppard |
| ENSMUSG00000002289 | 528.0137926 | 1865.693265 | 2130.496573 | 2428.467877 | Angptl4 |
| ENSMUSG00000002831 | 51.42395197 | 228.0173379 | 574.117156 | 226.1088592 | Plin4 |
| ENSMUSG00000003032 | 44.99595798 | 155.5632305 | 107.5164856 | 114.5226689 | Klf4 |
| ENSMUSG00000003348 | 59.68851569 | 13.85152053 | 30.27163186 | 33.28009183 | Mob3a |
| ENSMUSG00000003500 | 52.34223683 | 174.742259 | 99.16569058 | 90.05201318 | Impdh1 |
| ENSMUSG00000003541 | 11.01941828 | 105.4846563 | 45.92937248 | 44.04718036 | Ier3 |
| ENSMUSG00000003848 | 135.9061588 | 522.0957737 | 291.2339755 | 260.3677772 | Nob1 |
| ENSMUSG00000004100 | 165.2912742 | 1249.833352 | 729.6507128 | 351.3986167 | Ppan |
| ENSMUSG00000004933 | 3.673139427 | 42.62006316 | 14.61389124 | 19.57652461 | Matk |
| ENSMUSG00000004951 | 114.7856071 | 237.6068521 | 636.7481184 | 278.9654756 | Hspb1 |
| ENSMUSG00000005148 | 9.182848567 | 19.17902842 | 27.14008374 | 22.5130033 | Klf5 |
| ENSMUSG00000005547 | 12101.15784 | 5271.036311 | 4402.956662 | 4447.78639 | Cyp2a5 |
| ENSMUSG00000005580 | 337.0105424 | 86.3056279 | 93.9464437 | 169.3369378 | Adcy9 |
| ENSMUSG00000006050 | 869.6157593 | 2542.286767 | 2021.936238 | 1365.462591 | Sra1 |
| ENSMUSG00000006134 | 678.6125091 | 225.8863347 | 252.6115486 | 375.8692724 | Crkl |
| ENSMUSG00000006517 | 1714.437827 | 1097.466626 | 579.3364028 | 613.7240464 | Mvd |
| ENSMUSG00000006587 | 21.1205517 | 10.65501579 | 2.087698749 | 4.894131151 | Snai3 |
| ENSMUSG00000006711 | 410.4733309 | 210.9693126 | 141.9635149 | 177.1675477 | D130043K22Rik |
| ENSMUSG00000006777 | 33.05825484 | 8.524012632 | 8.350794996 | 10.76708853 | Krt23 |
| ENSMUSG00000008153 | 185.4935411 | 96.96064369 | 120.0426781 | 78.30609842 | Clstn3 |
| ENSMUSG00000009013 | 192.8398199 | 329.2399879 | 396.6627623 | 281.9019543 | Dynll1 |
| ENSMUSG00000009633 | 1060.619009 | 2971.683904 | 3862.242686 | 1956.673634 | G0s2 |
| ENSMUSG00000014547 | 108.3576131 | 21.31003158 | 36.53472811 | 46.98365905 | Wdfy2 |
| ENSMUSG00000014609 | 34.89482455 | 14.91702211 | 14.61389124 | 15.66121968 | Chrne |
| ENSMUSG00000015224 | 79.89078253 | 318.5849721 | 255.7430968 | 158.5698493 | Cyp2j9 |
| ENSMUSG00000015312 | 10.10113342 | 121.46718 | 59.49941435 | 63.62370497 | Gadd45b |
| ENSMUSG00000016128 | 631.7799814 | 199.2487953 | 218.1645193 | 304.4149576 | Stard13 |
| ENSMUSG00000016356 | 46.83252769 | 90.56763421 | 172.2351468 | 124.3109312 | Col20a1 |
| ENSMUSG00000017737 | 5.50970914 | 38.35805684 | 32.35933061 | 22.5130033 | Mmp9 |
| ENSMUSG00000017868 | 1179.077756 | 543.4058053 | 586.6433485 | 498.2225512 | Sgk2 |
| ENSMUSG00000018486 | 27.5485457 | 3.196504737 | 15.65774062 | 6.851783612 | Wnt9b |
| ENSMUSG00000019082 | 2721.796315 | 7723.820946 | 7053.290223 | 5201.482588 | Slc25a22 |
| ENSMUSG00000019726 | 627.1885571 | 179.0042653 | 147.1827618 | 268.1983871 | Lyst |
| ENSMUSG00000019737 | 6.427993997 | 45.8165679 | 15.65774062 | 18.59769837 | Syne4 |
| ENSMUSG00000019883 | 707.0793397 | 200.3142968 | 230.6907118 | 405.2340593 | Echdc1 |
| ENSMUSG00000020018 | 117.5404617 | 268.5063979 | 250.5238499 | 170.3157641 | Snrpf |
| ENSMUSG00000020027 | 291.0962996 | 2138.461669 | 846.5618427 | 589.2533906 | Socs2 |
| ENSMUSG00000020091 | 1814.530877 | 393.1700826 | 471.8199173 | 1129.56547 | Eif4ebp2 |
| ENSMUSG00000020122 | 689.6319274 | 2670.146957 | 1807.947117 | 1885.219319 | Egfr |
| ENSMUSG00000020335 | 80.80906739 | 24.50653632 | 41.75397498 | 32.3012656 | Zfp354b |
| ENSMUSG00000020429 | 977.0550875 | 2643.509417 | 2014.629293 | 3936.839098 | Igfbp1 |
| ENSMUSG00000020441 | 87.23706139 | 303.66795 | 273.4885361 | 168.3581116 | 2310033P09Rik |
| ENSMUSG00000020532 | 5607.047335 | 2810.793165 | 1739.053058 | 2484.260972 | Acaca |
| ENSMUSG00000020641 | 139.5792982 | 408.0871047 | 425.8905448 | 452.2177184 | Rsad2 |
| ENSMUSG00000020656 | 23.87540627 | 71.38860579 | 76.20100434 | 73.41196727 | Grhl1 |
| ENSMUSG00000020681 | 52.34223683 | 14.91702211 | 21.92083686 | 12.72474099 | Ace |
| ENSMUSG00000020692 | 76.21764311 | 249.3273695 | 152.4020087 | 104.7344066 | Nle1 |
| ENSMUSG00000020812 | 47.75081255 | 180.0697668 | 73.06945621 | 57.75074759 | 1810032O08Rik |
| ENSMUSG00000020889 | 1803.511459 | 443.2486569 | 371.6103773 | 433.62002 | Nr1d1 |
| ENSMUSG00000020917 | 22782.64729 | 9135.610538 | 6014.660096 | 10059.39717 | Acly |
| ENSMUSG00000020948 | 56.93366111 | 9.589514211 | 20.87698749 | 44.04718036 | Klhl28 |
| ENSMUSG00000020961 | 141.4158679 | 25.5720379 | 35.49087873 | 64.6025312 | Ston2 |
| ENSMUSG00000021250 | 25.71197599 | 204.5763032 | 115.8672806 | 135.0780198 | Fos |
| ENSMUSG00000021260 | 23.87540627 | 6.393009474 | 11.48234312 | 10.76708853 | Hhipl1 |
| ENSMUSG00000021416 | 629.0251268 | 404.8906 | 324.6371555 | 254.4948199 | Eci3 |
| ENSMUSG00000021453 | 130.3964497 | 979.1959511 | 568.8979091 | 678.3265776 | Gadd45g |
| ENSMUSG00000021611 | 475.6715558 | 220.5588268 | 227.5591636 | 197.7228985 | Tert |
| ENSMUSG00000021670 | 5239.733392 | 3051.596522 | 1273.496237 | 2047.704474 | Hmgcr |
| ENSMUSG00000021684 | 22.03883656 | 54.34058053 | 72.02560684 | 49.92013774 | Pde8b |
| ENSMUSG00000021773 | 22.03883656 | 44.75106632 | 55.32401685 | 29.36478691 | Comtd1 |
| ENSMUSG00000021775 | 1119.38924 | 635.0389411 | 274.5323855 | 351.3986167 | Nr1d2 |
| ENSMUSG00000021804 | 37.64967912 | 7.458511053 | 11.48234312 | 20.55535084 | Rgr |
| ENSMUSG00000021958 | 47.75081255 | 56.47158369 | 91.85874496 | 47.96248528 | Pinx1 |
| ENSMUSG00000022383 | 1669.441869 | 440.0521521 | 565.766361 | 794.806899 | Ppara |
| ENSMUSG00000022389 | 1561.084256 | 599.877389 | 502.0915491 | 601.9781316 | Tef |
| ENSMUSG00000022408 | 82.6456371 | 38.35805684 | 32.35933061 | 21.53417707 | Fam83f |
| ENSMUSG00000022528 | 197.4312442 | 643.5629537 | 588.7310472 | 500.1802037 | Hes1 |
| ENSMUSG00000022651 | 0 | 52.20957737 | 37.57857748 | 32.3012656 | Retnlg |
| ENSMUSG00000022704 | 100.0930494 | 291.9474326 | 188.9367368 | 134.0991935 | Qtrt2 |
| ENSMUSG00000022853 | 7460.146176 | 4279.054341 | 3944.706786 | 4891.194673 | Ehhadh |
| ENSMUSG00000022883 | 355.3762395 | 166.2182463 | 116.9111299 | 103.7555804 | Robo1 |
| ENSMUSG00000022887 | 1538.127135 | 350.5500195 | 374.7419254 | 819.2775547 | Masp1 |
| ENSMUSG00000022911 | 193.7581048 | 105.4846563 | 73.06945621 | 75.36961973 | Arl13b |
| ENSMUSG00000023034 | 22.95712142 | 572.1743479 | 134.6565693 | 149.7604132 | Nr4a1 |
| ENSMUSG00000023044 | 2315.914409 | 4109.63959 | 5560.585618 | 729O.297763 | Csad |
| ENSMUSG00000023052 | 17.44741228 | 36.22705368 | 52.19246872 | 20.55535084 | Npff |
| ENSMUSG00000023067 | 134.9878739 | 1114.514652 | 1009.402345 | 858.4306039 | Cdkn1a |
| ENSMUSG00000023073 | 592.2937326 | 369.7290479 | 140.9196656 | 157.5910231 | Slc10a2 |
| ENSMUSG00000023341 | 85.40049167 | 264.2443916 | 178.498243 | 164.4428067 | Mx2 |
| ENSMUSG00000023571 | 14.69255771 | 53.27507895 | 58.45556497 | 25.44948199 | C1qtnf12 |
| ENSMUSG00000023800 | 456.3875738 | 160.8907384 | 217.1206699 | 156.6121968 | Tiam2 |
| ENSMUSG00000023905 | 81.72735225 | 328.1744863 | 207.7260255 | 109.6285378 | Tnfrsf12a |
| ENSMUSG00000023927 | 12.85598799 | 61.79909158 | 60.54326372 | 33.28009183 | Satb1 |
| ENSMUSG00000023968 | 15.61084256 | 20.24453 | 38.62242686 | 33.28009183 | Crip3 |
| ENSMUSG00000024118 | 352.621385 | 1463.99917 | 1896.674313 | 1329.246021 | Tedc2 |
| ENSMUSG00000024130 | 3530.805274 | 1006.898992 | 1228.610714 | 2261.088592 | Abca3 |
| ENSMUSG00000024136 | 17.44741228 | 64.99559632 | 52.19246872 | 34.25891806 | Dnase1l2 |
| ENSMUSG00000024190 | 460.0607132 | 1247.702349 | 1464.520672 | 1004.275712 | Dusp1 |
| ENSMUSG00000024236 | 1299.373072 | 484.8032184 | 345.514143 | 575.5498234 | Svil |
| ENSMUSG00000024411 | 35.81310941 | 14.91702211 | 13.57004187 | 17.61887214 | Aqp4 |
| ENSMUSG00000024440 | 147.8438619 | 35.16155211 | 70.98175747 | 72.43314104 | Pcdh12 |
| ENSMUSG00000024665 | 9906.457034 | 3625.901873 | 3398.773563 | 5157.435407 | Fads2 |
| ENSMUSG00000024843 | 1263.559963 | 3171.998201 | 2942.611387 | 2249.342677 | Chka |
| ENSMUSG00000024887 | 565.6634717 | 191.7902842 | 179.5420924 | 296.5843478 | Asah2 |
| ENSMUSG00000024924 | 589.538878 | 269.5718995 | 262.006193 | 320.0761773 | Vldlr |
| ENSMUSG00000024970 | 25.71197599 | 90.56763421 | 63.67481184 | 65.58135743 | Al846148 |
| ENSMUSG00000024978 | 8311.396238 | 1324.418463 | 791.2378259 | 2079.026913 | Gpam |
| ENSMUSG00000025003 | 410.4733309 | 152.3667258 | 144.0512137 | 186.95581 | Cyp2c39 |
| ENSMUSG00000025006 | 416.9013249 | 107.6156595 | 110.6480337 | 207.5111608 | Sorbs1 |
| ENSMUSG00000025153 | 57005.28733 | 35454.56504 | 23677.63536 | 26074.95195 | Fasn |
| ENSMUSG00000025161 | 14.69255771 | 61.79909158 | 26.09623436 | 32.3012656 | Slc16a3 |
| ENSMUSG00000025240 | 696.0599214 | 183.2662716 | 163.8843518 | 418.9376266 | Sacm1l |
| ENSMUSG00000025323 | 172.6375531 | 71.38860579 | 52.19246872 | 60.68722628 | Sp4 |
| ENSMUSG00000025402 | 214.8786565 | 646.7594584 | 568.8979091 | 502.1378561 | Nab2 |
| ENSMUSG00000025429 | 651.9822483 | 188.5937795 | 118.9988287 | 253.5159936 | Pstpip2 |
| ENSMUSG00000025450 | 55.0970914 | 145.9737163 | 103.3410881 | 105.7132329 | Gm9752 |
| ENSMUSG00000025997 | 12.85598799 | 39.42355842 | 58.45556497 | 22.5130033 | Ikzf2 |
| ENSMUSG00000026020 | 269.9757479 | 828.9602284 | 520.8808379 | 466.9001118 | Nop58 |
| ENSMUSG00000026249 | 26.63026084 | 45.8165679 | 122.1303768 | 117.4591476 | Serpine2 |
| ENSMUSG00000026358 | 12.85598799 | 104.4191547 | 58.45556497 | 96.9037968 | Rgs1 |
| ENSMUSG00000026398 | 1539.04542 | 615.8599126 | 631.5288716 | 846.6846892 | Nr5a2 |
| ENSMUSG00000026471 | 315.8899907 | 181.1352684 | 104.3849374 | 126.2685837 | Mr1 |
| ENSMUSG00000026475 | 994.5024998 | 1039.929541 | 2826.744106 | 2390.293654 | Rgs16 |
| ENSMUSG00000026525 | 6.427993997 | 43.68556474 | 19.83313812 | 16.64004591 | Opn3 |
| ENSMUSG00000026822 | 110.1941828 | 304.7334516 | 223.3837661 | 189.8922887 | Lcn2 |
| ENSMUSG00000026826 | 11.93770314 | 69.25760263 | 25.05238499 | 13.70356722 | Nr4a2 |
| ENSMUSG00000026832 | 25.71197599 | 101.22265 | 42.79782435 | 62.64487874 | Cytip |
| ENSMUSG00000027360 | 72.54450368 | 370.7945495 | 319.4179086 | 181.0828526 | Hdc |
| ENSMUSG00000027398 | 32.13996998 | 98.02614527 | 82.46410059 | 128.2262362 | Il1b |
| ENSMUSG00000027405 | 248.8551962 | 920.5933642 | 450.9429298 | 352.3774429 | Nop56 |
| ENSMUSG00000027496 | 17.44741228 | 55.40608211 | 66.80635997 | 66.56018366 | Aurka |
| ENSMUSG00000027513 | 18901.97549 | 48173.45739 | 29794.5927 | 39823.54518 | Pck1 |
| ENSMUSG00000027605 | 21561.32844 | 8380.169919 | 3799.611723 | 6353.561061 | Acss2 |
| ENSMUSG00000027762 | 1404.975831 | 637.1699442 | 555.3278672 | 690.0724923 | Sucnr1 |
| ENSMUSG00000027907 | 106.5210434 | 207.7728079 | 117.9549793 | 139.9721509 | S100a11 |
| ENSMUSG00000027947 | 461.8972829 | 713.8860579 | 763.0538928 | 1358.610808 | Il6ra |
| ENSMUSG00000028008 | 55.0970914 | 20.24453 | 24.00853561 | 36.21657052 | Asic5 |
| ENSMUSG00000028339 | 521.5857986 | 295.1439374 | 274.5323855 | 289.7325642 | Col15a1 |
| ENSMUSG00000028445 | 91.82848567 | 91.63313579 | 280.7954817 | 425.7894102 | Enho |
| ENSMUSG00000028630 | 406.8001915 | 124.6636847 | 101.2533893 | 226.1088592 | Dyrk2 |
| ENSMUSG00000028838 | 198.349529 | 74.58511053 | 49.0609206 | 78.30609842 | Extl1 |
| ENSMUSG00000028859 | 29.38511541 | 152.3667258 | 96.03414245 | 74.3907935 | Csf3r |
| ENSMUSG00000028862 | 17.44741228 | 77.78161526 | 40.71012561 | 57.75074759 | Map3k6 |
| ENSMUSG00000028864 | 203.8592382 | 39.42355842 | 69.93790809 | 111.5861902 | Hgf |
| ENSMUSG00000028957 | 341.6019667 | 62.86459316 | 53.2363181 | 46.00483282 | Per3 |
| ENSMUSG00000028976 | 101.9296191 | 42.62006316 | 34.44702936 | 45.02600659 | Slc2a5 |
| ENSMUSG00000029086 | 593.2120174 | 210.9693126 | 165.9720505 | 243.7277313 | Prom1 |
| ENSMUSG00000029135 | 136.8244436 | 499.7202405 | 272.4446867 | 294.6266953 | Fosl2 |
| ENSMUSG00000029188 | 51.42395197 | 186.4627763 | 179.5420924 | 182.0616788 | Slc34a2 |
| ENSMUSG00000029195 | 788.8066919 | 332.4364926 | 209.8137243 | 422.8529315 | Klb |
| ENSMUSG00000029370 | 579.4377446 | 190.7247826 | 137.7881174 | 169.3369378 | Rassf6 |
| ENSMUSG00000029373 | 9.182848567 | 25.5720379 | 28.18393311 | 18.59769837 | Pf4 |
| ENSMUSG00000029380 | 71.62621882 | 615.8599126 | 454.0744779 | 700.8395809 | Cxcl1 |
| ENSMUSG00000029580 | 6927.540959 | 16369.30076 | 20515.81561 | 13266.0319 | Actb |
| ENSMUSG00000029591 | 35.81310941 | 177.9387637 | 131.5250212 | 112.5650165 | Ung |
| ENSMUSG00000029656 | 189.1666805 | 429.3971363 | 614.8272816 | 554.0156463 | C8b |
| ENSMUSG00000030032 | 25.71197599 | 43.68556474 | 55.32401685 | 35.23774429 | Wdr54 |
| ENSMUSG00000030055 | 292.9328693 | 75.65061211 | 74.11330559 | 171.2945903 | Rab43 |
| ENSMUSG00000030691 | 415.0647552 | 189.6592811 | 180.5859418 | 227.0876854 | Fchsd2 |
| ENSMUSG00000030782 | 40.40453369 | 115.0741705 | 67.85020934 | 70.47548858 | Tgfb1i1 |
| ENSMUSG00000030814 | 291.0962996 | 814.0432063 | 869.526529 | 459.069502 | Bcl7c |
| ENSMUSG00000030827 | 33.05825484 | 120.4016784 | 80.37640184 | 48.94131151 | Fgf21 |
| ENSMUSG00000030934 | 9134.17947 | 5865.586192 | 5683.759844 | 6052.082582 | Oat |
| ENSMUSG00000030968 | 54.17880654 | 131.0566942 | 132.5688706 | 93.9673181 | Pdilt |
| ENSMUSG00000031010 | 1420.586673 | 414.4801142 | 361.1718836 | 775.2303744 | Usp9x |
| ENSMUSG00000031271 | 364.5590881 | 1418.182602 | 1115.874981 | 1330.224847 | Serpina7 |
| ENSMUSG00000031378 | 885.2266019 | 429.3971363 | 392.4873648 | 441.4506298 | Abcd1 |
| ENSMUSG00000031465 | 20.20226685 | 50.07857421 | 30.27163186 | 47.96248528 | Angpt2 |
| ENSMUSG00000031762 | 32.13996998 | 9477.636545 | 4672.2698 | 1607.23267 | Mt2 |
| ENSMUSG00000031765 | 337.0105424 | 20312.7221 | 10174.39985 | 4243.211708 | Mt1 |
| ENSMUSG00000032009 | 1044.089882 | 377.187559 | 338.2071973 | 562.8250824 | Sesn3 |
| ENSMUSG00000032064 | 661.1650968 | 268.5063979 | 302.7163186 | 381.7422298 | Dixdc1 |
| ENSMUSG00000032083 | 68671.17815 | 163165.5843 | 112907.9676 | 104422.1611 | Apoa1 |
| ENSMUSG00000032091 | 32.13996998 | 20.24453 | 8.350794996 | 4.894131151 | Tmprss4 |
| ENSMUSG00000032285 | 52.34223683 | 26.63753947 | 15.65774062 | 44.04718036 | Dnaja4 |
| ENSMUSG00000032417 | 80.80906739 | 26.63753947 | 43.84167373 | 17.61887214 | Rwdd2a |
| ENSMUSG00000032418 | 13799.06654 | 6053.11447 | 3739.068459 | 4136.519649 | Me1 |
| ENSMUSG00000032500 | 372.8236518 | 75.65061211 | 69.93790809 | 123.332105 | Dclk3 |
| ENSMUSG00000032561 | 1176.322901 | 453.9036726 | 141.9635149 | 317.1396986 | Acpp |
| ENSMUSG00000032702 | 1494.049462 | 468.8206947 | 629.4411728 | 864.3035613 | Kank1 |
| ENSMUSG00000032724 | 680.4490788 | 234.4103474 | 176.4105443 | 323.9914822 | Abtb2 |
| ENSMUSG00000032735 | 294.769439 | 436.8556474 | 741.1330559 | 606.8722628 | Ablim3 |
| ENSMUSG00000032786 | 2206.638511 | 7150.581096 | 4035.521682 | 3301.580875 | Alas1 |
| ENSMUSG00000032849 | 399.4539127 | 168.3492495 | 143.0073643 | 195.7652461 | Abcc4 |
| ENSMUSG00000032860 | 290.1780147 | 155.5632305 | 134.6565693 | 141.9298034 | P2ry2 |
| ENSMUSG00000032883 | 1528.944286 | 856.6632695 | 580.3802522 | 723.3525842 | Acsl3 |
| ENSMUSG00000033105 | 3240.627259 | 1574.811334 | 996.8761526 | 1567.100795 | Lss |
| ENSMUSG00000033594 | 348.9482455 | 852.4012632 | 820.4656084 | 539.3332529 | Spata2l |
| ENSMUSG00000033624 | 428.8390281 | 105.4846563 | 98.1218412 | 178.1463739 | Pdpr |
| ENSMUSG00000033792 | 74.38107339 | 21.31003158 | 26.09623436 | 43.06835413 | Atp7a |
| ENSMUSG00000033855 | 198.349529 | 37.29255526 | 42.79782435 | 77.32727219 | Ston1 |
| ENSMUSG00000033967 | 1.836569713 | 15.98252368 | 19.83313812 | 39.15304921 | Rnf225 |
| ENSMUSG00000034066 | 310.3802816 | 96.96064369 | 107.5164856 | 184.9981575 | Farp2 |
| ENSMUSG00000034110 | 115.7038919 | 55.40608211 | 37.57857748 | 55.79309512 | Kctd7 |
| ENSMUSG00000034271 | 40.40453369 | 135.3187005 | 37.57857748 | 64.6025312 | Jdp2 |
| ENSMUSG00000034755 | 12.85598799 | 19.17902842 | 36.53472811 | 20.55535084 | Pcdh11x |
| ENSMUSG00000034765 | 9.182848567 | 166.2182463 | 53.2363181 | 31.32243937 | Dusp5 |
| ENSMUSG00000034853 | 434.3487372 | 165.1527447 | 137.7881174 | 147.8027608 | Acot11 |
| ENSMUSG00000034926 | 17627.39611 | 6118.110066 | 6691.074491 | 12774.66113 | Dhcr24 |
| ENSMUSG00000035078 | 346.193391 | 94.82964053 | 101.2533893 | 258.4101248 | Mtmr9 |
| ENSMUSG00000035112 | 18.36569713 | 43.68556474 | 45.92937248 | 36.21657052 | Wnk4 |
| ENSMUSG00000035164 | 141.4158679 | 61.79909158 | 70.98175747 | 77.32727219 | Zc3h12c |
| ENSMUSG00000035165 | 28.46683056 | 144.9082147 | 55.32401685 | 91.03083941 | Kcne3 |
| ENSMUSG00000035284 | 1017.459621 | 293.0129342 | 218.1645193 | 482.5613315 | Vps13c |
| ENSMUSG00000035900 | 367.3139427 | 147.0392179 | 160.7528037 | 191.8499411 | Gramd4 |
| ENSMUSG00000035933 | 244.2637719 | 77.78161526 | 81.42025121 | 127.2474099 | Cog5 |
| ENSMUSG00000035948 | 857.6780562 | 193.9212874 | 154.4897074 | 418.9376266 | Acss3 |
| ENSMUSG00000036062 | 3.673139427 | 27.70304105 | 29.22778249 | 12.72474099 | Phf24 |
| ENSMUSG00000036120 | 224.9797899 | 773.5541463 | 626.3096247 | 392.5093183 | Rfxank |
| ENSMUSG00000036611 | 585.8657386 | 415.5456158 | 267.2254399 | 309.3090888 | Eepd1 |
| ENSMUSG00000037035 | 50.50566712 | 141.71171 | 149.2704606 | 192.8287674 | Inhbb |
| ENSMUSG00000037071 | 340820.5063 | 157237.1335 | 63623.66322 | 106805.6029 | Scd1 |
| ENSMUSG00000037095 | 2991.772063 | 5413.813523 | 8132.630477 | 6142.134595 | Lrg1 |
| ENSMUSG00000037157 | 58.77023083 | 11.72051737 | 14.61389124 | 24.47065576 | Il22ra1 |
| ENSMUSG00000037336 | 8.26456371 | 23.44103474 | 42.79782435 | 31.32243937 | Mfsd2b |
| ENSMUSG00000037443 | 1089.08584 | 3091.020081 | 2288.117829 | 2056.51391 | Cep85 |
| ENSMUSG00000037447 | 25.71197599 | 99.09164684 | 54.28016747 | 54.81426889 | Arid5a |
| ENSMUSG00000037465 | 297.5242936 | 592.4188779 | 817.3340602 | 760.5479809 | Klf10 |
| ENSMUSG00000037583 | 213.9603716 | 304.7334516 | 631.5288716 | 569.676866 | Nr0b2 |
| ENSMUSG00000037709 | 857.6780562 | 239.7378553 | 289.1462767 | 357.271574 | Fam13a |
| ENSMUSG00000037887 | 11.01941828 | 80.97812 | 20.87698749 | 30.34361314 | Dusp8 |
| ENSMUSG00000038217 | 3049.624009 | 1346.793996 | 1375.793476 | 1970.377202 | Tlcd2 |
| ENSMUSG00000038233 | 856.7597713 | 284.4889216 | 256.7869461 | 354.3350954 | Fam198a |
| ENSMUSG00000038253 | 14.69255771 | 112.9431674 | 83.50794996 | 44.04718036 | Hoxa5 |
| ENSMUSG00000038370 | 141.4158679 | 277.0304105 | 627.3534741 | 676.3689251 | Pcp4l1 |
| ENSMUSG00000038415 | 205.6958079 | 331.3709911 | 448.855231 | 695.9454497 | Foxq1 |
| ENSMUSG00000038418 | 131.3147345 | 515.7027642 | 342.3825948 | 912.2660466 | Egr1 |
| ENSMUSG00000038473 | 89.99191596 | 45.8165679 | 35.49087873 | 46.98365905 | Nos1ap |
| ENSMUSG00000038530 | 13.77427285 | 64.99559632 | 30.27163186 | 63.62370497 | Rgs4 |
| ENSMUSG00000038583 | 11.93770314 | 67.12659947 | 41.75397498 | 16.64004591 | Pln |
| ENSMUSG00000038587 | 141.4158679 | 349.4845179 | 176.4105443 | 253.5159936 | Akap12 |
| ENSMUSG00000038751 | 171.7192682 | 46.88206947 | 44.8855231 | 44.04718036 | Ptk6 |
| ENSMUSG00000038768 | 124.8867405 | 106.5501579 | 77.24485371 | 93.9673181 | 9130409l23Rik |
| ENSMUSG00000038774 | 488.5275438 | 194.986789 | 189.9805862 | 328.8856134 | Ascc3 |
| ENSMUSG00000038844 | 386.5979247 | 169.4147511 | 146.1389124 | 248.6218625 | Kif16b |
| ENSMUSG00000038895 | 94.58334024 | 230.1483411 | 226.5153143 | 190.8711149 | Zfp653 |
| ENSMUSG00000039103 | 26.63026084 | 68.19210105 | 74.11330559 | 43.06835413 | Nexn |
| ENSMUSG00000039304 | 181.8204016 | 105.4846563 | 73.06945621 | 81.24257711 | Tnfsf10 |
| ENSMUSG00000039533 | 293.8511541 | 167.2837479 | 54.28016747 | 122.3532788 | Mmd2 |
| ENSMUSG00000039601 | 732.7913156 | 337.7640005 | 298.5409211 | 280.9231281 | Rcan2 |
| ENSMUSG00000039704 | 463.7338526 | 59.66808842 | 50.10476998 | 182.0616788 | Lmbrd2 |
| ENSMUSG00000039741 | 292.0145844 | 105.4846563 | 146.1389124 | 137.0356722 | Bahcc1 |
| ENSMUSG00000039853 | 491.2823983 | 126.7946879 | 156.5774062 | 258.4101248 | Trim14 |
| ENSMUSG00000039981 | 147.8438619 | 53.27507895 | 77.24485371 | 62.64487874 | Zc3h12d |
| ENSMUSG00000040093 | 357.2128093 | 75.65061211 | 69.93790809 | 132.1415411 | Bmf |
| ENSMUSG00000040128 | 1381.100424 | 3368.050491 | 2671.210549 | 2388.336002 | Pnrc1 |
| ENSMUSG00000040152 | 35.81310941 | 91.63313579 | 40.71012561 | 85.15788203 | Thbs1 |
| ENSMUSG00000040435 | 127.6415951 | 430.4626379 | 241.1292055 | 201.6382034 | Ppp1r15a |
| ENSMUSG00000040584 | 406.8001915 | 118.2706753 | 124.2180756 | 233.939469 | Abcb1a |
| ENSMUSG00000040855 | 645.5542543 | 259.9823853 | 223.3837661 | 409.1493642 | Reps2 |
| ENSMUSG00000040891 | 360.8859487 | 1650.461946 | 1922.770548 | 1525.990093 | Foxa3 |
| ENSMUSG00000041134 | 84.48220682 | 18.11352684 | 31.31548123 | 39.15304921 | Cyyr1 |
| ENSMUSG00000041372 | 28.46683056 | 9.589514211 | 12.52619249 | 9.788262303 | B4galnt3 |
| ENSMUSG00000041695 | 23.87540627 | 101.22265 | 78.28870309 | 41.11070167 | Kcnj2 |
| ENSMUSG00000041702 | 179.0655471 | 59.66808842 | 58.45556497 | 97.88262303 | Btbd7 |
| ENSMUSG00000041920 | 162.5364196 | 586.0258684 | 355.9526367 | 312.2455675 | Slc16a6 |
| ENSMUSG00000041930 | 29.38511541 | 157.6942337 | 126.3057743 | 71.45431481 | Fam222a |
| ENSMUSG00000041945 | 20.20226685 | 3.196504737 | 5.219246872 | 10.76708853 | Mfsd9 |
| ENSMUSG00000042010 | 7079.05796 | 4018.006454 | 2149.285862 | 2550.821156 | Acacb |
| ENSMUSG00000042115 | 51.42395197 | 117.2051737 | 124.2180756 | 131.1627149 | Klhdc8a |
| ENSMUSG00000042246 | 23.87540627 | 77.78161526 | 33.40317998 | 54.81426889 | Tmc7 |
| ENSMUSG00000042333 | 64.27993997 | 30.89954579 | 27.14008374 | 41.11070167 | Tnfrsf14 |
| ENSMUSG00000042354 | 269.057463 | 922.7243674 | 424.8466954 | 389.5728396 | Gnl3 |
| ENSMUSG00000042379 | 89.99191596 | 140.6462084 | 79.33255246 | 203.5958559 | Esm1 |
| ENSMUSG00000042444 | 674.0210848 | 255.720379 | 221.2960674 | 319.0973511 | Mindy2 |
| ENSMUSG00000042510 | 26.63026084 | 94.82964053 | 45.92937248 | 47.96248528 | AA986860 |
| ENSMUSG00000042607 | 14.69255771 | 37.29255526 | 24.00853561 | 42.0895279 | Asb4 |
| ENSMUSG00000042622 | 31.22168513 | 89.50213263 | 50.10476998 | 72.43314104 | Maff |
| ENSMUSG00000042680 | 358.1310941 | 87.37112948 | 114.8234312 | 243.7277313 | Garem1 |
| ENSMUSG00000042743 | 50.50566712 | 15.98252368 | 14.61389124 | 26.42830822 | Sgtb |
| ENSMUSG00000042745 | 317.7265604 | 726.6720769 | 889.3596671 | 654.834748 | Id1 |
| ENSMUSG00000043165 | 9.182848567 | 13.85152053 | 41.75397498 | 38.17422298 | Lor |
| ENSMUSG00000043421 | 33.9765397 | 138.5152053 | 55.32401685 | 75.36961973 | Hilpda |
| ENSMUSG00000043639 | 87.23706139 | 20.24453 | 46.97322185 | 49.92013774 | Rbm20 |
| ENSMUSG00000043681 | 202.0226685 | 543.4058053 | 474.9514654 | 382.721056 | Fam25c |
| ENSMUSG00000044042 | 353.5396698 | 108.6811611 | 85.59564871 | 203.5958559 | Fmn1 |
| ENSMUSG00000044186 | 30.30340027 | 17.04802526 | 10.43849374 | 17.61887214 | Nkx2-6 |
| ENSMUSG00000044339 | 28.46683056 | 61.79909158 | 89.77104621 | 79.28492465 | Alkbh2 |
| ENSMUSG00000044349 | 182.7386865 | 148.1047195 | 83.50794996 | 50.89896397 | Snhg11 |
| ENSMUSG00000044359 | 263.5477539 | 69.25760263 | 76.20100434 | 148.781587 | P2ry4 |
| ENSMUSG00000044676 | 146.0072922 | 609.4669032 | 360.1280342 | 270.1560396 | Zfp612 |
| ENSMUSG00000044749 | 3631.816608 | 1050.584557 | 874.7457758 | 2096.645785 | Abca6 |
| ENSMUSG00000044948 | 23.87540627 | 73.51960895 | 52.19246872 | 59.70840005 | Cfap43 |
| ENSMUSG00000045045 | 171.7192682 | 38.35805684 | 38.62242686 | 56.77192136 | Lrfn4 |
| ENSMUSG00000045294 | 18334.47545 | 8335.418852 | 7066.860265 | 6456.337815 | Insig1 |
| ENSMUSG00000045348 | 9.182848567 | 38.35805684 | 44.8855231 | 20.55535084 | Nyap1 |
| ENSMUSG00000045382 | 23.87540627 | 89.50213263 | 68.89405872 | 29.36478691 | Cxcr4 |
| ENSMUSG00000045411 | 227.7346445 | 798.0606826 | 403.9697079 | 303.4361314 | 2410002F23Rik |
| ENSMUSG00000045776 | 1214.890865 | 514.6372626 | 321.5056073 | 424.8105839 | Lrtm1 |
| ENSMUSG00000045875 | 117.5404617 | 28.76854263 | 45.92937248 | 72.43314104 | Adra1a |
| ENSMUSG00000046541 | 84.48220682 | 22.37553316 | 22.96468624 | 41.11070167 | Zfp526 |
| ENSMUSG00000046721 | 22.95712142 | 46.88206947 | 46.97322185 | 42.0895279 | Rpl14-ps1 |
| ENSMUSG00000046908 | 10.10113342 | 36.22705368 | 28.18393311 | 24.47065576 | Ltb4r1 |
| ENSMUSG00000047496 | 451.7961495 | 124.6636847 | 234.8661093 | 298.5420002 | Rnf152 |
| ENSMUSG00000047649 | 64.27993997 | 210.9693126 | 155.5335568 | 111.5861902 | Cd3eap |
| ENSMUSG00000047875 | 85.40049167 | 49.01307263 | 46.97322185 | 47.96248528 | Gpr157 |
| ENSMUSG00000048191 | 11.01941828 | 53.27507895 | 40.71012561 | 27.40713445 | Muc6 |
| ENSMUSG00000048644 | 8.26456371 | 40.48906 | 37.57857748 | 23.49182953 | Ctxn1 |
| ENSMUSG00000048856 | 4005.558545 | 8878.824658 | 12487.57007 | 11318.1677 | Slc25a47 |
| ENSMUSG00000049044 | 1266.314817 | 3411.736056 | 3040.733228 | 2890.473858 | Rapgef4 |
| ENSMUSG00000049313 | 312.2168513 | 152.3667258 | 109.6041843 | 170.3157641 | Sorl1 |
| ENSMUSG00000049580 | 479.3446952 | 1707.999031 | 1943.647535 | 1562.206663 | Tsku |
| ENSMUSG00000049791 | 759.4215765 | 215.231319 | 290.1901261 | 450.2600659 | Fzd4 |
| ENSMUSG00000049950 | 21.1205517 | 94.82964053 | 44.8855231 | 35.23774429 | Rpp38 |
| ENSMUSG00000050390 | 1292.026793 | 692.5760263 | 634.6604197 | 674.4112726 | C77080 |
| ENSMUSG00000050503 | 7.346278854 | 25.5720379 | 38.62242686 | 16.64004591 | Fbxl22 |
| ENSMUSG00000050663 | 44.07767312 | 15.98252368 | 13.57004187 | 15.66121968 | Trhde |
| ENSMUSG00000050737 | 44.99595798 | 119.3361768 | 55.32401685 | 31.32243937 | Ptges |
| ENSMUSG00000050914 | 14.69255771 | 273.8339058 | 58.45556497 | 46.98365905 | Ankrd37 |
| ENSMUSG00000051149 | 39.48624884 | 6.393009474 | 3.131548123 | 21.53417707 | Adnp |
| ENSMUSG00000051339 | 1850.343986 | 917.3968595 | 914.4120521 | 942.6096597 | 2900026A02Rik |
| ENSMUSG00000051452 | 283.7500207 | 92.69863737 | 80.37640184 | 127.2474099 | Gm11437 |
| ENSMUSG00000051674 | 608.82286 | 295.1439374 | 270.356988 | 333.7797445 | Dcun1d4 |
| ENSMUSG00000051998 | 6.427993997 | 42.62006316 | 39.66627623 | 21.53417707 | Lax1 |
| ENSMUSG00000052085 | 446.2864404 | 197.1177921 | 161.796653 | 227.0876854 | Dock8 |
| ENSMUSG00000052595 | 2847.601341 | 821.5017174 | 661.8005034 | 1485.858218 | A1cf |
| ENSMUSG00000052656 | 3382.043127 | 1950.933391 | 1650.325861 | 1785.379044 | Rnf103 |
| ENSMUSG00000052684 | 181.8204016 | 547.6678116 | 268.2692892 | 399.3611019 | Jun |
| ENSMUSG00000052713 | 102.8479039 | 23.44103474 | 18.78928874 | 42.0895279 | Zfp608 |
| ENSMUSG00000052837 | 276.4037419 | 1233.850828 | 697.2913822 | 1027.767542 | Junb |
| ENSMUSG00000053560 | 242.4272022 | 725.6065753 | 569.9417585 | 550.1003414 | Ier2 |
| ENSMUSG00000053964 | 153.3535711 | 348.4190163 | 621.0903778 | 388.5940134 | Lgals4 |
| ENSMUSG00000053977 | 7.346278854 | 57.53708526 | 21.92083686 | 40.13187544 | Cd8a |
| ENSMUSG00000054008 | 3392.144261 | 753.3096163 | 842.3864452 | 2156.354185 | Ndst1 |
| ENSMUSG00000054150 | 78.97249768 | 33.03054895 | 29.22778249 | 32.3012656 | Syne3 |
| ENSMUSG00000054422 | 27702.81756 | 57173.74923 | 60050.56682 | 33819.42508 | Fabp1 |
| ENSMUSG00000054453 | 126.7233102 | 69.25760263 | 36.53472811 | 39.15304921 | Sytl5 |
| ENSMUSG00000054659 | 100.0930494 | 15.98252368 | 25.05238499 | 65.58135743 | Pm20d2 |
| ENSMUSG00000054932 | 233.2443536 | 72.45410737 | 72.02560684 | 80.26375088 | Afp |
| ENSMUSG00000055148 | 137.7427285 | 476.2792058 | 390.3996661 | 293.6478691 | Klf2 |
| ENSMUSG00000055254 | 2139.603716 | 409.1526063 | 418.5835992 | 554.9944726 | Ntrk2 |
| ENSMUSG00000055491 | 144.1707225 | 706.4275469 | 442.5921348 | 309.3090888 | Pprc1 |
| ENSMUSG00000055660 | 112.0307525 | 29.83404421 | 36.53472811 | 60.68722628 | Mettl4 |
| ENSMUSG00000055692 | 19.28398199 | 90.56763421 | 57.4117156 | 44.04718036 | Tmem191c |
| ENSMUSG00000055980 | 381.0882155 | 114.0086689 | 159.7089543 | 270.1560396 | Irs1 |
| ENSMUSG00000056054 | 4.591424283 | 100.1571484 | 98.1218412 | 95.92497057 | S100a8 |
| ENSMUSG00000056071 | 12.85598799 | 140.6462084 | 203.550628 | 123.332105 | S100a9 |
| ENSMUSG00000056091 | 2190.109383 | 3671.718441 | 3506.290049 | 4394.929774 | St3gal5 |
| ENSMUSG00000056148 | 71.62621882 | 23.44103474 | 25.05238499 | 50.89896397 | Rdh9 |
| ENSMUSG00000056313 | 415.0647552 | 1762.339612 | 990.6130564 | 874.0918236 | Tcim |
| ENSMUSG00000057342 | 1236.011417 | 564.7158369 | 644.0550641 | 614.7028726 | Sphk2 |
| ENSMUSG00000057604 | 11.93770314 | 25.5720379 | 28.18393311 | 27.40713445 | Lmcd1 |
| ENSMUSG00000057722 | 110.1941828 | 323.91248 | 199.3752305 | 166.4004591 | Lepr |
| ENSMUSG00000057969 | 99.17476452 | 49.01307263 | 42.79782435 | 37.19539675 | Sema3b |
| ENSMUSG00000058207 | 28330.9244 | 48372.70618 | 79528.79614 | 62218.1105 | Serpina3k |
| ENSMUSG00000058503 | 153.3535711 | 376.1220574 | 383.0927204 | 227.0876854 | Fam133b |
| ENSMUSG00000058793 | 3164.409616 | 1397.938072 | 1320.469459 | 1959.610113 | Cds2 |
| ENSMUSG00000058794 | 144.1707225 | 71.38860579 | 26.09623436 | 30.34361314 | Nfe2 |
| ENSMUSG00000058921 | 1263.559963 | 261.0478868 | 329.8564023 | 683.2207087 | Slc10a5 |
| ENSMUSG00000059149 | 348.9482455 | 147.0392179 | 80.37640184 | 237.854774 | Mfsd4a |
| ENSMUSG00000059824 | 248.8551962 | 24.50653632 | 16.70158999 | 12.72474099 | Dbp |
| ENSMUSG00000060429 | 944.9151175 | 201.3797984 | 180.5859418 | 380.7634036 | Sntb1 |
| ENSMUSG00000061175 | 989.9110755 | 495.4582342 | 405.0135573 | 478.6460266 | Fnip2 |
| ENSMUSG00000061292 | 1350.797024 | 523.1612753 | 399.7943104 | 986.6568401 | Cyp3a59 |
| ENSMUSG00000061436 | 663.9199514 | 228.0173379 | 186.849038 | 275.0501707 | Hipk2 |
| ENSMUSG00000061536 | 199.2678139 | 88.43663105 | 94.99029308 | 144.8662821 | Sec22c |
| ENSMUSG00000061825 | 678.6125091 | 298.3404421 | 160.7528037 | 130.1838886 | Ces2c |
| ENSMUSG00000062901 | 992.6659301 | 297.2749405 | 259.9184942 | 513.8837709 | Klhl24 |
| ENSMUSG00000063535 | 70.70793397 | 27.70304105 | 29.22778249 | 32.3012656 | Zfp773 |
| ENSMUSG00000063704 | 224.061505 | 581.7638621 | 784.9747296 | 446.344761 | Mapk15 |
| ENSMUSG00000063929 | 82.6456371 | 208.8383095 | 167.0158999 | 174.231069 | Cyp4a32 |
| ENSMUSG00000065126 | 40.40453369 | 154.497729 | 158.6651049 | 77.32727219 | Snord104 |
| ENSMUSG00000065147 | 15.61084256 | 54.34058053 | 32.35933061 | 19.57652461 | Snora31 |
| ENSMUSG00000065952 | 188.2483956 | 51.14407579 | 55.32401685 | 76.34844596 | C330021F23Rik |
| ENSMUSG00000066456 | 150.5987165 | 70.32310421 | 34.44702936 | 38.17422298 | Hmgn3 |
| ENSMUSG00000066477 | 49.58738226 | 93.76413895 | 118.9988287 | 119.4168001 | Gm16551 |
| ENSMUSG00000066687 | 161.6181348 | 472.0171995 | 943.6398345 | 1137.39608 | Zbtb16 |
| ENSMUSG00000066944 | 40.40453369 | 10.65501579 | 10.43849374 | 24.47065576 | NA |
| ENSMUSG00000067149 | 162.5364196 | 437.921149 | 878.9211733 | 284.838433 | Jchain |
| ENSMUSG00000068463 | 5.50970914 | 46.88206947 | 15.65774062 | 12.72474099 | B630019A10Rik |
| ENSMUSG00000068742 | 691.4684971 | 312.1919626 | 260.9623436 | 334.7585707 | Cry2 |
| ENSMUSG00000068877 | 136.8244436 | 201.3797984 | 256.7869461 | 255.4736461 | Selenbp2 |
| ENSMUSG00000069456 | 5731.934075 | 2190.671246 | 1455.126028 | 1756.014257 | Rdh16 |
| ENSMUSG00000069804 | 13.77427285 | 42.62006316 | 55.32401685 | 34.25891806 | Gm10277 |
| ENSMUSG00000070576 | 226.8163596 | 98.02614527 | 132.5688706 | 122.3532788 | Mn1 |
| ENSMUSG00000070583 | 40.40453369 | 13.85152053 | 11.48234312 | 31.32243937 | Fv1 |
| ENSMUSG00000071076 | 938.4871235 | 3733.517533 | 2948.874483 | 1847.045097 | Jund |
| ENSMUSG00000071456 | 48.6690974 | 12.78601895 | 16.70158999 | 35.23774429 | 1110002L01Rik |
| ENSMUSG00000071547 | 47.75081255 | 90.56763421 | 104.3849374 | 70.47548858 | Nt5dc2 |
| ENSMUSG00000071637 | 89.99191596 | 239.7378553 | 123.1742262 | 171.2945903 | Cebpd |
| ENSMUSG00000071645 | 188.2483956 | 605.2048969 | 525.0562354 | 343.5680068 | Tut1 |
| ENSMUSG00000072294 | 196.5129593 | 24.50653632 | 18.78928874 | 60.68722628 | Klf12 |
| ENSMUSG00000072571 | 55.0970914 | 20.24453 | 29.22778249 | 29.36478691 | Tmem253 |
| ENSMUSG00000072664 | 1131.326943 | 273.8339058 | 440.504436 | 724.3314104 | Ugt3a1 |
| ENSMUSG00000072692 | 28.46683056 | 73.51960895 | 70.98175747 | 58.72957382 | Rpl37rt |
| ENSMUSG00000072849 | 102.8479039 | 570.0433447 | 342.3825948 | 378.8057511 | Serpina1e |
| ENSMUSG00000072999 | 168.0461288 | 294.0784358 | 417.5397498 | 310.287915 | Gm15401 |
| ENSMUSG00000073460 | 10.10113342 | 51.14407579 | 79.33255246 | 37.19539675 | Pnldc1 |
| ENSMUSG00000073835 | 325.0728393 | 85.24012632 | 69.93790809 | 16.64004591 | Mup-ps12 |
| ENSMUSG00000074024 | 53.26052169 | 118.2706753 | 117.9549793 | 98.86144926 | 4632427E13Rik |
| ENSMUSG00000074063 | 1493.131177 | 2843.823714 | 6567.900264 | 5870.020903 | Osgin1 |
| ENSMUSG00000074213 | 37.64967912 | 24.50653632 | 27.14008374 | 18.59769837 | Gm10642 |
| ENSMUSG00000074345 | 56.93366111 | 26.63753947 | 24.00853561 | 29.36478691 | Tnfaip8l3 |
| ENSMUSG00000074375 | 1850.343986 | 860.9252758 | 876.8334746 | 855.4941252 | Sult2a3 |
| ENSMUSG00000074876 | 117.5404617 | 71.38860579 | 41.75397498 | 54.81426889 | Spata5l1 |
| ENSMUSG00000075470 | 422.4110341 | 77.78161526 | 80.37640184 | 251.5583412 | Deaf1 |
| ENSMUSG00000075552 | 2640.068963 | 1023.947017 | 947.815232 | 894.6471745 | Cyp3a41b |
| ENSMUSG00000075590 | 507.8115258 | 1682.426993 | 1456.169877 | 1087.475942 | Nrbp2 |
| ENSMUSG00000076490 | 8.26456371 | 35.16155211 | 20.87698749 | 18.59769837 | Trbc1 |
| ENSMUSG00000076569 | 46.83252769 | 72.45410737 | 110.6480337 | 183.0405051 | Igkv5-39 |
| ENSMUSG00000076596 | 9.182848567 | 152.3667258 | 28.18393311 | 26.42830822 | Igkv3-10 |
| ENSMUSG00000076609 | 704.3244851 | 4135.211628 | 4467.675323 | 1384.06029 | Igkc |
| ENSMUSG00000076613 | 74.38107339 | 100.1571484 | 155.5335568 | 99.84027549 | Ighg2b |
| ENSMUSG00000076617 | 1859.526835 | 5037.691465 | 5276.658588 | 2480.345667 | Ighm |
| ENSMUSG00000076934 | 10.10113342 | 77.78161526 | 36.53472811 | 58.72957382 | Iglv1 |
| ENSMUSG00000077148 | 69.78964911 | 9.589514211 | 19.83313812 | 11.74591476 | Gm22935 |
| ENSMUSG00000078193 | 21.1205517 | 42.62006316 | 35.49087873 | 30.34361314 | Gm2000 |
| ENSMUSG00000078234 | 599.6400114 | 197.1177921 | 263.0500424 | 351.3986167 | Klhdc7a |
| ENSMUSG00000078650 | 4180.950953 | 6922.563759 | 10279.82864 | 10371.64274 | G6pc |
| ENSMUSG00000078651 | 16.52912742 | 55.40608211 | 57.4117156 | 43.06835413 | Aoc2 |
| ENSMUSG00000078672 | 152.4352862 | 337.7640005 | 374.7419254 | 237.854774 | Mup20 |
| ENSMUSG00000078688 | 173.5558379 | 49.01307263 | 38.62242686 | 39.15304921 | Mup2 |
| ENSMUSG00000078817 | 80.80906739 | 204.5763032 | 280.7954817 | 342.5891806 | Nlrp12 |
| ENSMUSG00000079017 | 44.07767312 | 145.9737163 | 124.2180756 | 88.09436072 | Ifi27l2a |
| ENSMUSG00000079036 | 106.5210434 | 575.3708526 | 288.1024274 | 180.1040264 | Alkbh1 |
| ENSMUSG00000079065 | 125.8050254 | 371.8600511 | 353.864938 | 194.7864198 | BC005561 |
| ENSMUSG00000079465 | 22.03883656 | 46.88206947 | 56.36786622 | 36.21657052 | Col4a3 |
| ENSMUSG00000079470 | 474.7532709 | 201.3797984 | 169.1035987 | 244.7065576 | Utp14b |
| ENSMUSG00000080059 | 6.427993997 | 37.29255526 | 13.57004187 | 19.57652461 | Rps19-ps3 |
| ENSMUSG00000081344 | 38.56796398 | 64.99559632 | 55.32401685 | 43.06835413 | Gm14303 |
| ENSMUSG00000082065 | 255.2831902 | 60.73359 | 39.66627623 | 5.872957382 | Mup-ps14 |
| ENSMUSG00000082173 | 224.061505 | 41.55456158 | 31.31548123 | 8.809436072 | Mup-ps10 |
| ENSMUSG00000082586 | 47.75081255 | 15.98252368 | 7.306945621 | 17.61887214 | Sult2a-ps1 |
| ENSMUSG00000082658 | 23.87540627 | 85.24012632 | 129.4373224 | 49.92013774 | Fau-ps2 |
| ENSMUSG00000083327 | 82.6456371 | 36.22705368 | 34.44702936 | 66.56018366 | Vcp-rs |
| ENSMUSG00000083621 | 20.20226685 | 54.34058053 | 48.01707123 | 31.32243937 | Gm14586 |
| ENSMUSG00000083716 | 35.81310941 | 70.32310421 | 86.63949808 | 39.15304921 | Gm13436 |
| ENSMUSG00000083813 | 22.03883656 | 86.3056279 | 84.55179933 | 92.00966564 | Gm15502 |
| ENSMUSG00000083863 | 27.5485457 | 64.99559632 | 54.28016747 | 47.96248528 | Gm13341 |
| ENSMUSG00000083992 | 20.20226685 | 39.42355842 | 40.71012561 | 29.36478691 | Gm11478 |
| ENSMUSG00000084822 | 13.77427285 | 66.0610979 | 91.85874496 | 58.72957382 | Myadml2os |
| ENSMUSG00000084883 | 126.7233102 | 24.50653632 | 30.27163186 | 63.62370497 | Ccdc85c |
| ENSMUSG00000085001 | 21.1205517 | 156.6287321 | 70.98175747 | 70.47548858 | Rapgef4os2 |
| ENSMUSG00000085156 | 16.52912742 | 157.6942337 | 59.49941435 | 34.25891806 | Snhg15 |
| ENSMUSG00000085445 | 72.54450368 | 126.7946879 | 143.0073643 | 187.9346362 | Gm16348 |
| ENSMUSG00000085834 | 71.62621882 | 1120.907661 | 744.264604 | 450.2600659 | Gm15622 |
| ENSMUSG00000085995 | 353.5396698 | 444.3141584 | 772.4485371 | 450.2600659 | Gm2788 |
| ENSMUSG00000086140 | 44.99595798 | 11.72051737 | 22.96468624 | 25.44948199 | Hnf1aos2 |
| ENSMUSG00000086446 | 9.182848567 | 27.70304105 | 97.07799183 | 26.42830822 | Prkag2os1 |
| ENSMUSG00000086529 | 44.07767312 | 13.85152053 | 8.350794996 | 2.936478691 | Acss2os |
| ENSMUSG00000086786 | 33.05825484 | 2.131003158 | 3.131548123 | 15.66121968 | Gm15908 |
| ENSMUSG00000086844 | 18.36569713 | 73.51960895 | 42.79782435 | 46.98365905 | B230206H07Rik |
| ENSMUSG00000087382 | 136.8244436 | 414.4801142 | 369.5226786 | 445.3659348 | Ctcflos |
| ENSMUSG00000087445 | 15.61084256 | 61.79909158 | 54.28016747 | 39.15304921 | Gm14286 |
| ENSMUSG00000087595 | 17.44741228 | 41.55456158 | 62.63096247 | 36.21657052 | 1810012K08Rik |
| ENSMUSG00000087613 | 82.6456371 | 12.78601895 | 17.74543937 | 48.94131151 | Gm13855 |
| ENSMUSG00000087616 | 8.26456371 | 154.497729 | 34.44702936 | 26.42830822 | Gm14257 |
| ENSMUSG00000087658 | 9.182848567 | 36.22705368 | 28.18393311 | 23.49182953 | Hotairm1 |
| ENSMUSG00000089726 | 45.91424283 | 293.0129342 | 129.4373224 | 86.13670826 | Mir17hg |
| ENSMUSG00000089943 | 3147.880489 | 781.0126574 | 515.661591 | 1296.944755 | Ugt1a5 |
| ENSMUSG00000090021 | 19.28398199 | 15.98252368 | 39.66627623 | 48.94131151 | Gm6493 |
| ENSMUSG00000090145 | 606.0680054 | 154.497729 | 220.252218 | 375.8692724 | Ugt1a6b |
| ENSMUSG00000090175 | 415.0647552 | 125.7291863 | 209.8137243 | 409.1493642 | Ugt1a9 |
| ENSMUSG00000090264 | 21.1205517 | 57.53708526 | 80.37640184 | 52.85661643 | Eif4ebp3 |
| ENSMUSG00000090369 | 106.5210434 | 27.70304105 | 27.14008374 | 41.11070167 | 4933411K16Rik |
| ENSMUSG00000090555 | 103.7661888 | 1396.87257 | 624.2219259 | 491.3707676 | Gm8893 |
| ENSMUSG00000090610 | 47.75081255 | 35.16155211 | 28.18393311 | 22.5130033 | Gm3571 |
| ENSMUSG00000090698 | 24.79369113 | 247.1963663 | 60.54326372 | 76.34844596 | Apold1 |
| ENSMUSG00000091021 | 18.36569713 | 82.04362158 | 27.14008374 | 23.49182953 | Gm17300 |
| ENSMUSG00000091509 | 68.87136425 | 143.8427132 | 183.7174899 | 79.28492465 | Gm17066 |
| ENSMUSG00000092075 | 0 | 83.10912316 | 163.8843518 | 57.75074759 | Serpina4-ps1 |
| ENSMUSG00000094410 | 116.6221768 | 47.94757105 | 25.05238499 | 56.77192136 | Gm38394 |
| ENSMUSG00000095280 | 24.79369113 | 126.7946879 | 158.6651049 | 55.79309512 | Gm21738 |
| ENSMUSG00000095351 | 166.2095591 | 629.7114332 | 1051.15632 | 311.2667412 | Igkv3-2 |
| ENSMUSG00000096833 | 23.87540627 | 3.196504737 | 4.175397498 | 9.788262303 | Igkv4-55 |
| ENSMUSG00000096910 | 72.54450368 | 34.09605053 | 10.43849374 | 48.94131151 | Zfp955b |
| ENSMUSG00000096954 | 81.72735225 | 33.03054895 | 17.74543937 | 37.19539675 | Gdap10 |
| ENSMUSG00000097124 | 130.3964497 | 23.44103474 | 33.40317998 | 25.44948199 | A530020G20Rik |
| ENSMUSG00000097221 | 42.24110341 | 8.524012632 | 9.39464437 | 27.40713445 | 1810049J17Rik |
| ENSMUSG00000097312 | 33.9765397 | 114.0086689 | 161.796653 | 45.02600659 | Gm26870 |
| ENSMUSG00000097536 | 40.40453369 | 13.85152053 | 26.09623436 | 18.59769837 | 2610037D02Rik |
| ENSMUSG00000097615 | 25.71197599 | 69.25760263 | 65.76251059 | 70.47548858 | Gm2061 |
| ENSMUSG00000097660 | 6.427993997 | 60.73359 | 53.2363181 | 10.76708853 | Gm26762 |
| ENSMUSG00000097691 | 175.3924076 | 75.65061211 | 98.1218412 | 102.7767542 | 9030616G12Rik |
| ENSMUSG00000097743 | 50.50566712 | 155.5632305 | 98.1218412 | 88.09436072 | Gm16973 |
| ENSMUSG00000097908 | 98.25647967 | 52.20957737 | 45.92937248 | 57.75074759 | 4933404O12Rik |
| ENSMUSG00000097971 | 1313.147345 | 8345.008366 | 6440.550641 | 3776.311596 | Gm26917 |
| ENSMUSG00000097994 | 11.93770314 | 29.83404421 | 48.01707123 | 38.17422298 | Gm26982 |
| ENSMUSG00000098041 | 17.44741228 | 68.19210105 | 69.93790809 | 30.34361314 | Gm26981 |
| ENSMUSG00000098661 | 14.69255771 | 43.68556474 | 48.01707123 | 36.21657052 | Mir7052 |
| ENSMUSG00000098814 | 1.836569713 | 13.85152053 | 157.6212555 | 4.894131151 | Igkv19-93 |
| ENSMUSG00000098882 | 93.66505538 | 37.29255526 | 18.78928874 | 10.76708853 | Mir6392 |
| ENSMUSG00000099568 | 19.28398199 | 18.11352684 | 59.49941435 | 47.96248528 | Gm28513 |
| ENSMUSG00000099858 | 168.0461288 | 56.47158369 | 75.15715496 | 114.5226689 | Gm6652 |
| ENSMUSG00000100094 | 3195.631301 | 1017.554008 | 731.7384115 | 815.3622498 | 1810008I18Rik |
| ENSMUSG00000100468 | 34.89482455 | 6.393009474 | 6.263096247 | 9.788262303 | Tmem167-ps1 |
| ENSMUSG00000101939 | 21.1205517 | 52.20957737 | 56.36786622 | 46.00483282 | Gm28438 |
| ENSMUSG00000102275 | 14.69255771 | 56.47158369 | 54.28016747 | 18.59769837 | Gm37144 |
| ENSMUSG00000102577 | 82.6456371 | 137.4497037 | 252.6115486 | 199.680551 | Gm37969 |
| ENSMUSG00000102719 | 4.591424283 | 38.35805684 | 30.27163186 | 8.809436072 | Gm37760 |
| ENSMUSG00000102869 | 361.8042335 | 86.3056279 | 81.42025121 | 216.3205969 | 2900097C17Rik |
| ENSMUSG00000102882 | 216.7152262 | 26.63753947 | 38.62242686 | 15.66121968 | Gm2065 |
| ENSMUSG00000102918 | 66.11650968 | 18.11352684 | 14.61389124 | 27.40713445 | Pcdhgc3 |
| ENSMUSG00000103285 | 8.26456371 | 27.70304105 | 35.49087873 | 14.68239345 | Gm37274 |
| ENSMUSG00000103546 | 6.427993997 | 28.76854263 | 33.40317998 | 17.61887214 | Gm37666 |
| ENSMUSG00000104030 | 5.50970914 | 56.47158369 | 39.66627623 | 21.53417707 | 5330406M23Rik |
| ENSMUSG00000104388 | 6.427993997 | 46.88206947 | 34.44702936 | 12.72474099 | Gm37033 |
| ENSMUSG00000104399 | 18.36569713 | 77.78161526 | 41.75397498 | 42.0895279 | Gm37963 |
| ENSMUSG00000104445 | 89.99191596 | 38.35805684 | 40.71012561 | 25.44948199 | Rhbg |
| ENSMUSG00000104973 | 4.591424283 | 36.22705368 | 32.35933061 | 32.3012656 | A530041M06Rik |
| ENSMUSG00000105161 | 13.77427285 | 44.75106632 | 39.66627623 | 23.49182953 | Gm42595 |
| ENSMUSG00000105434 | 6.427993997 | 26.63753947 | 42.79782435 | 12.72474099 | Gm43359 |
| ENSMUSG00000105547 | 8.26456371 | 23.44103474 | 27.14008374 | 61.66605251 | Iglc3 |
| ENSMUSG00000105556 | 33.05825484 | 7.458511053 | 9.39464437 | 12.72474099 | Gm43080 |
| ENSMUSG00000105703 | 160.6998499 | 712.8205563 | 708.7737253 | 409.1493642 | Gm43305 |
| ENSMUSG00000105881 | 252.5283356 | 43.68556474 | 106.4726362 | 160.5275018 | 4932422M17Rik |
| ENSMUSG00000105906 | 14.69255771 | 182.20077 | 62.63096247 | 23.49182953 | Iglc1 |
| ENSMUSG00000106030 | 198.349529 | 62.86459316 | 88.72719683 | 54.81426889 | Gm43611 |
| ENSMUSG00000106664 | 19.28398199 | 54.34058053 | 55.32401685 | 33.28009183 | Gm17936 |
| ENSMUSG00000106705 | 31.22168513 | 87.37112948 | 163.8843518 | 91.03083941 | Gm2602 |
| ENSMUSG00000106706 | 4.591424283 | 31.96504737 | 38.62242686 | 10.76708853 | C530043K16Rik |
| ENSMUSG00000106943 | 13.77427285 | 30.89954579 | 34.44702936 | 22.5130033 | Dancr |
| ENSMUSG00000107168 | 5.50970914 | 46.88206947 | 39.66627623 | 13.70356722 | Gm42507 |
| ENSMUSG00000107225 | 6.427993997 | 46.88206947 | 43.84167373 | 33.28009183 | Gm43637 |
| ENSMUSG00000107304 | 11.93770314 | 28.76854263 | 59.49941435 | 16.64004591 | Gm43775 |
| ENSMUSG00000107390 | 11.93770314 | 46.88206947 | 70.98175747 | 26.42830822 | Gm43323 |
| ENSMUSG00000107624 | 11.93770314 | 103.3536532 | 111.6918831 | 37.19539675 | Gm44005 |
| ENSMUSG00000108368 | 7.346278854 | 46.88206947 | 41.75397498 | 47.96248528 | Gm45053 |
| ENSMUSG00000108633 | 9.182848567 | 49.01307263 | 33.40317998 | 16.64004591 | Gm44694 |
| ENSMUSG00000108820 | 8.26456371 | 21.31003158 | 25.05238499 | 25.44948199 | Gm44620 |
| ENSMUSG00000108825 | 191.0032502 | 37.29255526 | 51.14861935 | 84.1790558 | Gm45838 |
| ENSMUSG00000109089 | 96.41990995 | 112.9431674 | 204.5944774 | 281.9019543 | 4833411C07Rik |
| ENSMUSG00000109115 | 19.28398199 | 4.262006316 | 4.175397498 | 8.809436072 | Gm44669 |
| ENSMUSG00000109157 | 8.26456371 | 25.5720379 | 30.27163186 | 18.59769837 | Gm44829 |
| ENSMUSG00000109262 | 24.79369113 | 14.91702211 | 19.83313812 | 12.72474099 | Gm44744 |
| ENSMUSG00000109291 | 9.182848567 | 17.04802526 | 38.62242686 | 25.44948199 | Gm2814 |
| ENSMUSG00000109536 | 29.38511541 | 109.7466626 | 90.81489558 | 68.51783612 | 9330162G02Rik |
| ENSMUSG00000109555 | 15.61084256 | 43.68556474 | 33.40317998 | 21.53417707 | Gm44891 |
| ENSMUSG00000109807 | 12.85598799 | 75.65061211 | 94.99029308 | 37.19539675 | Gm45244 |
| ENSMUSG00000109836 | 89.0736311 | 12.78601895 | 12.52619249 | 45.02600659 | Gm45884 |
| ENSMUSG00000109841 | 33.05825484 | 13.85152053 | 15.65774062 | 21.53417707 | E330011O21Rik |
| ENSMUSG00000110588 | 3.673139427 | 1747.42259 | 289.1462767 | 231.9818166 | Gm45774 |
| ENSMUSG00000110613 | 38.56796398 | 26.63753947 | 16.70158999 | 28.38596068 | Lncbate1 |
| ENSMUSG00000110702 | 8.26456371 | 45.8165679 | 30.27163186 | 25.44948199 | Gm45767 |
| ENSMUSG00000110755 | 176.3106925 | 79.91261842 | 28.18393311 | 20.55535084 | BC049987 |
| ENSMUSG00000111282 | 30.30340027 | 87.37112948 | 62.63096247 | 27.40713445 | Gm47528 |
| ENSMUSG00000111312 | 56.93366111 | 13.85152053 | 20.87698749 | 22.5130033 | Gm47205 |
| ENSMUSG00000111631 | 11.01941828 | 28.76854263 | 66.80635997 | 52.85661643 | Gm32017 |
| ENSMUSG00000111709 | 7.346278854 | 12.78601895 | 8.350794996 | 5.872957382 | Gm3776 |
| ENSMUSG00000111774 | 39.48624884 | 5.327507895 | 7.306945621 | 1.957652461 | AC166078.1 |

### Example 10

### TGFRt15-TGFRs Treatment Downregulates Genes Related to Glucose Metabolism, Lipid Mtabolism, and Amino Acid Metabolism in Liver

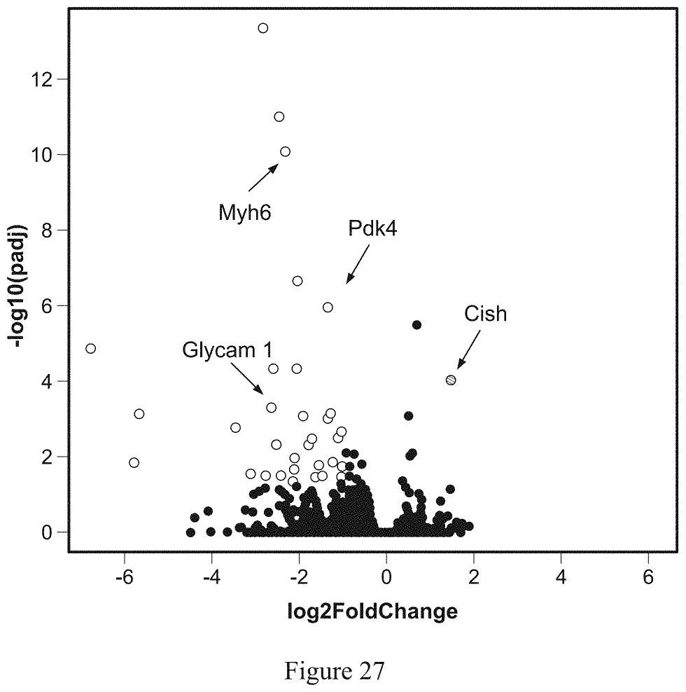

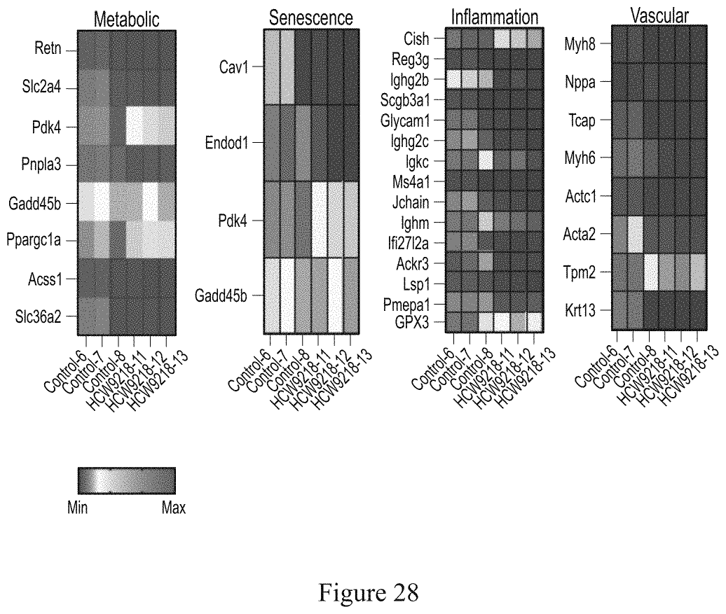

In light of the fact that type-II diabetes (T2D) is a metabolic disease and the liver is a key metabolic organ governing body energy metabolism, RNA-seq analysis on the livers of db/db mice was performed following TGFRt15-TGFRs treatment. Differentially expressed liver genes were detected in treated db/db mice and untreated control db/db mice. One gene was upregulated and 32 genes were downregulated, which together were grouped into four clusters based on function, as shown in FIG. 27. Expression of 8 genes related to glucose, lipid, or amino acid metabolism were significantly reduced in the liver following TGFRt15-TGFRs treatment, as shown in FIG. 28. For example,

Resistin (Retn) has been shown to induce insulin resistance in mice partially through toll-like receptor 4 signaling pathway and downregulation of Retn after TGFRt15-TGFRs treatment can contribute to the reduction of insulin resistance. As shown in FIG. 28, the expression of cellular senescence related genes, Cavl, Endodl, Pdk4, and Gadd45b, was also downregulated after TGFRt15-TGFRs treatment, indicating TGFRt15-TGFRs treatment can reduce senescent cell levels in livers. As shown in FIG. 28, fourteen pro-inflammation genes were downregulated and one gene was upregulated (Cish) indicating that TGFRt15-TGFRs treatment reduced liver inflammation. As shown in FIG. 28, expression of nine genes related to vascular regulation was also reduced. This result further indicates that the reduction of SNCs and SASP in db/db mice may favorably impact vascular health in diabetes. Taken together, these RNA-seq results indicate that TGFRt15-TGFRs treatment reduces the cellular senescence, SASP, and gluconeogenesis induced by metabolic dysfunction to improve glucose metabolism, metabolic homeostasis, and lower sterile inflammation in the livers of T2D db/db mice.

### Example 11

### Senescence-Associated Genes are Downregulated In Livers of Aged Mice Treated with TGFRt15-TGFRs

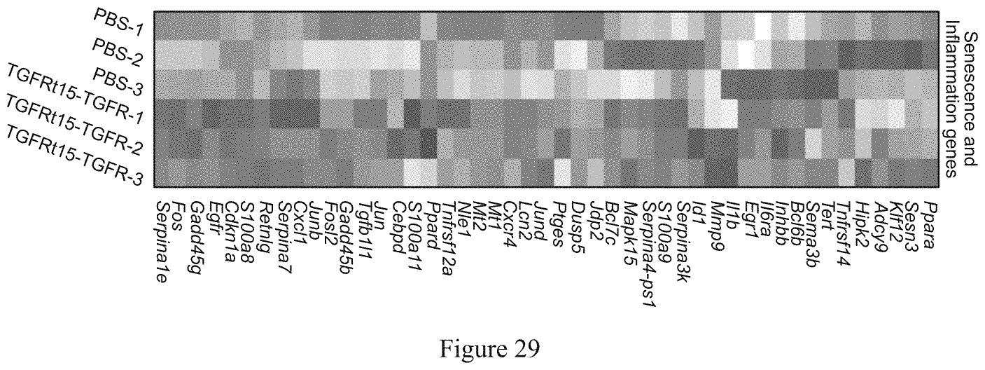

In order to investigate the effect of TGFRt15-TGFRs treatment on senescent cells (SNCs) and senescence-associated secretory phenotype (SASP) of peripheral organs, expression of inflammation and senescence-associate genes in aged mice (76 weeks) were interrogated by RNA-seq. Aged mice received either one or two subcutaneous doses of TGFRt15-TGFRs (3 mg/kg) or PBS (negative control). RNA-seq analysis was performed on the liver isolated at 60 or 90 days after TGFRt15-TGFRs treatment to determine the global transcriptional changes. Significant differentially expressed genes were clustered by their gene ontology and the enrichment of gene ontology terms was tested using Fisher exact test (GeneSCF v1.1-p2). The livers of TGFRt15-TGFRs-treated aged mice showed significant changes in gene expression with a total of 539 differentially expressed mRNAs compared to PBS-treated mice. As shown in FIG. 29, RNA-seq analysis indicated significant downregulation of genes including Cdkn1a, Nle1, Jund, Sema3b, Bcl6, Bcl7c, and Gadd45β and upregulation of senescence and inflammation associated genes (e.g., cytokines: Il6ra, Il1a, Il-6, Tnfa, S100a8, S100a9, S100a11 , Lcn2, Retnlg, Inhbb; chemokines: Cxcl1, Cxcr4, Mt1, and Mt2; metalloproteinases: Mmp9; gene expression and signaling pathways: e.g., Cebpd, Klf12, Egr1, Egfr, Gadd45β, Gadd45g, Ppara, Pparδ, Fos, Fosl2, Jun, Junb, Mapkl5, Adcy9).

### Example 12

### Cellular Senescence in Peripheral Organs of Aged Mice Treated with TGFRt15-TGFRs

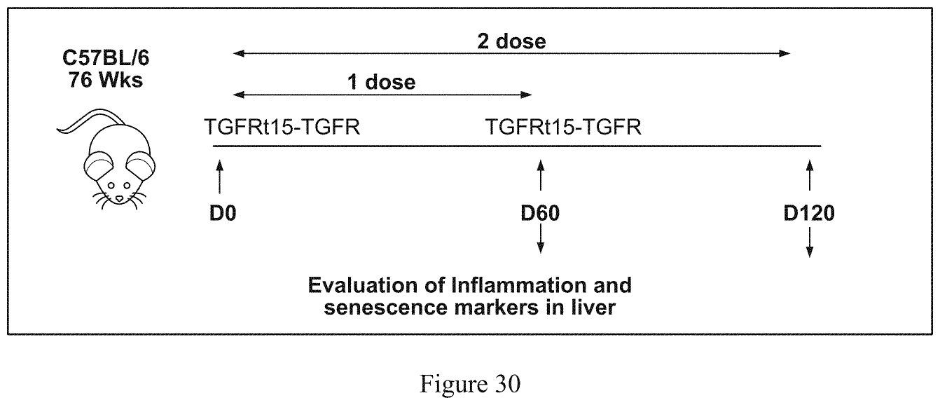

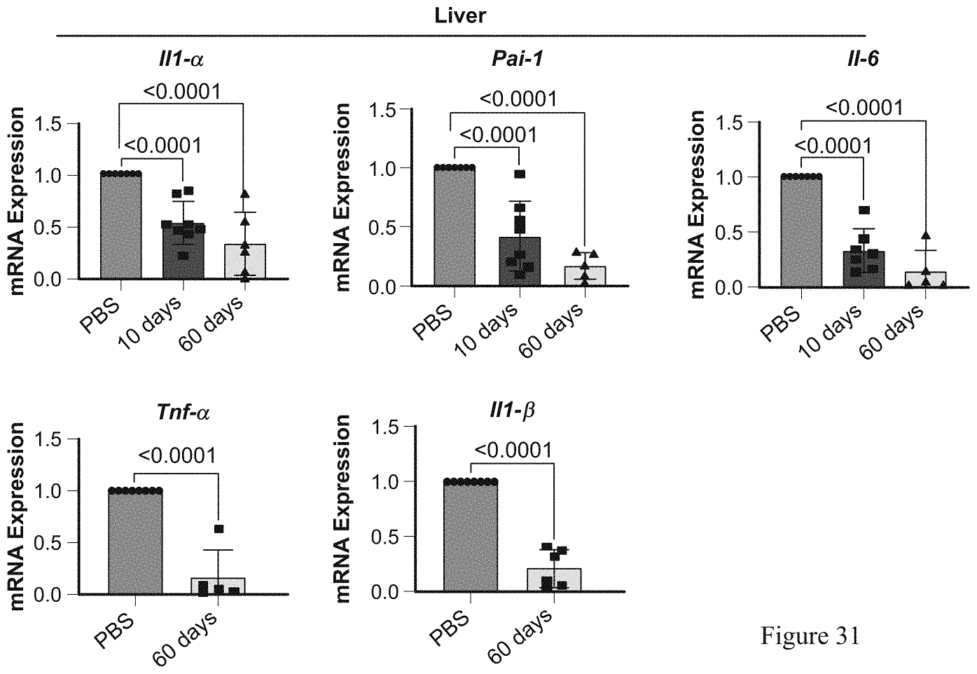

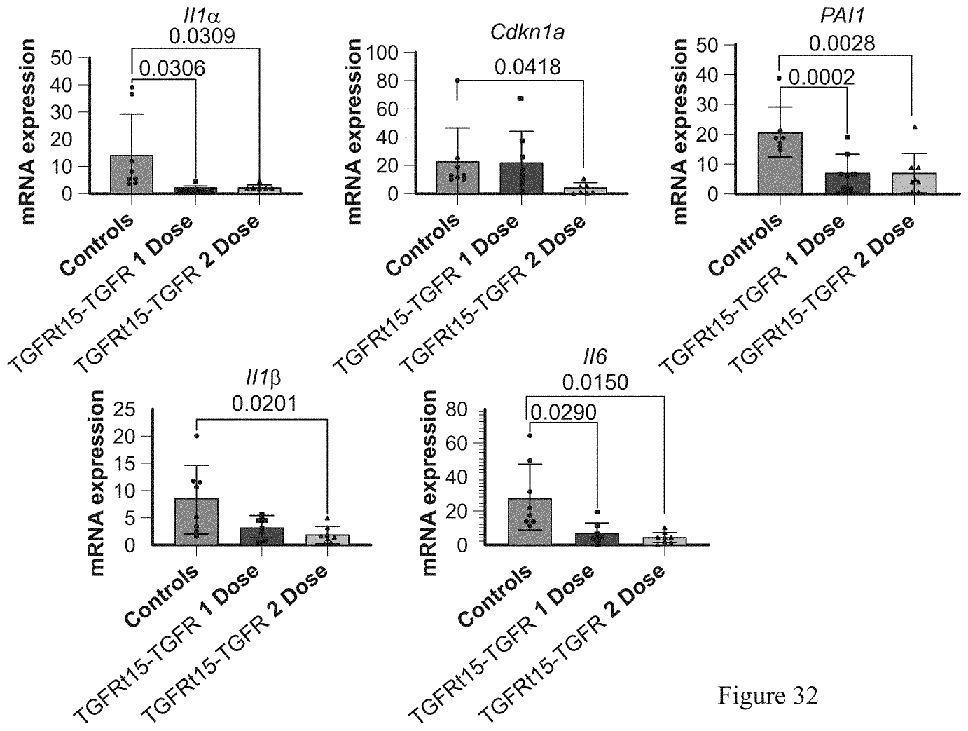

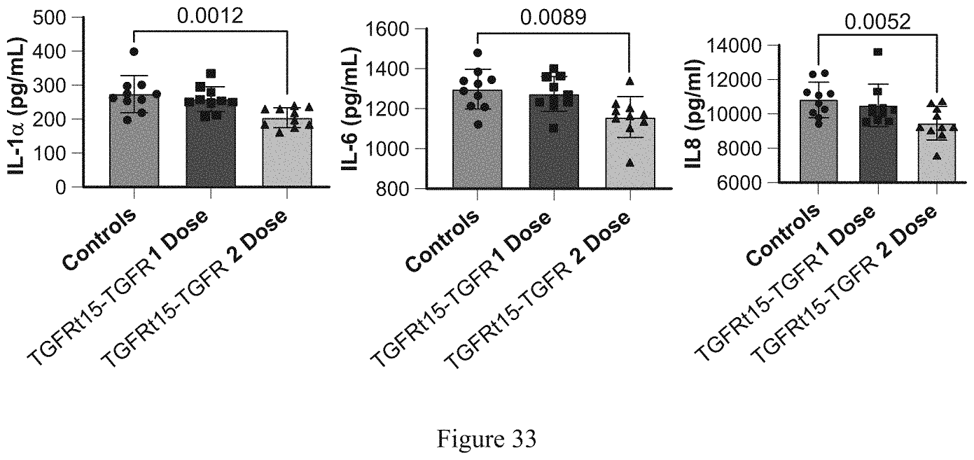

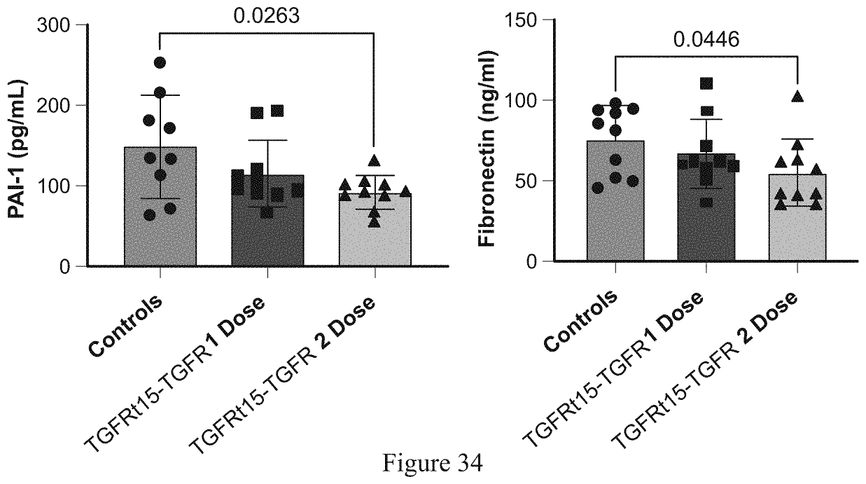

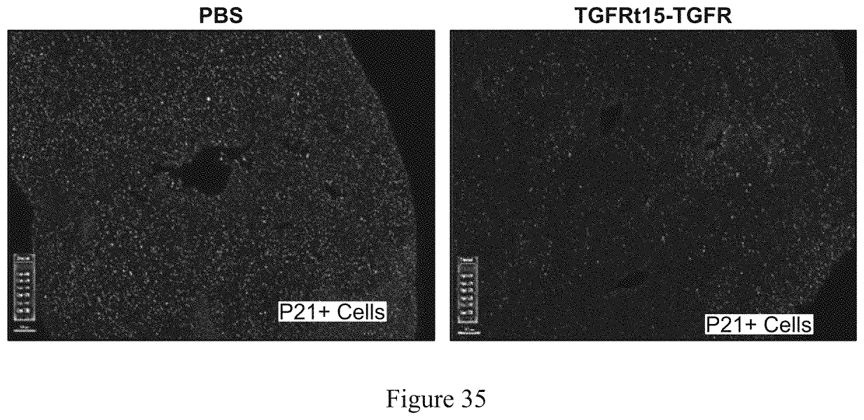

In order to further analyze the impacts of TGFRt15-TGFRs treatment on cellular senescence and senescence-associate secretory phenotype (SASP) in the peripheral organs of aged mice, qRT-PCR, ELISA, and immunofluorescence studies were performed for selected markers. As shown in FIG. 30, either one or two doses of TGFRt15-TGFRs treatment was given to aged mice. As shown in FIG. 31, qRT-PCR analysis of liver of aged mice either 10 days or 60 days after a single-dose TGFRt15-TGFRs treatment showed a significant reduction in gene expression for the cellular senescence and SASP signature genes, PAI-1, I1ia, Ilβ, 113, and Tnfa compared to the PBS control mice. As shown in FIG. 32, two-dose TGFRt15-TGFR treatment also provided significant reduction in Ilia, Cdkn1a, PAI, Il 1b, and Il6 transcripts in the liver at 120 days post-treatment initiation versus the control group. As shown in FIG. 33, reduction of liver IL-1α, IL-6 and IL-8 were also observed at protein levels by ELISA. As shown in FIG. 34, in a two-dose treatment regimen, treatment with TGFRt15-TGFRs lowered biomarkers PAI-1 and fibronectin, indicating that treatment with TGFRt15-TGFRs can reduce liver fibrosis in aged mice, consistent with significant down-regulation of Col4a3 and Col20a1 expression observed in the RNA-seq study of Example 10. As shown in FIG. 35, immunofluorescence staining of aged mice liver sections confirmed accumulation of p21+ SNCs which were reduced with TGFRt15-TGFRs treatment.

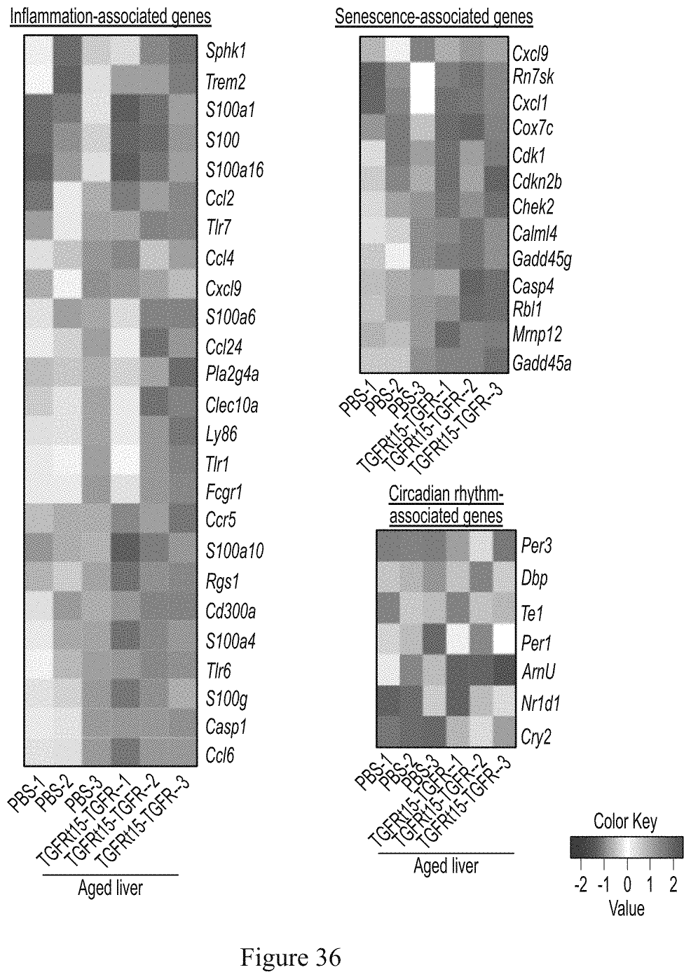

To further investigate the durability of the senolytic and senomorphic activities of TGFRt15-TGFRs treatment on gene expression in the livers of aged mice, RNA-seq studies were performed. Significant downregulation (e.g., Cdkn1a) or upregulation (e.g., Tert) of sensescence and inflammation associated (SASP) genes (e.g., cytokine: Il7, Il15, Il18, S100g, S100a1, S100a4, S100a6, S100a10, S100a16, S100g; chemokines: Ccl2, Clc4, Ccl6, Ccl7, Ccl8, Ccl9, Cc124, Ccl25, Ccl27, Cxcl1, Cxcl10, Cxcl11; metalloproteins: Mmp12, Mmp13, Mmp27; gene expression and signaling pathways: Klfl, Klf3, Klf7, Klf9, Klf13, Egrl, Ppara, Jun, Fos12; Mapk3, Mapk6, Mapk7, Mapk9, Mapk12, Mapk15, Adcyl, Adcy3, Adcy5, Adcy6, Adcy9, Adcyl0), and gene associated liver functions (e.g., Dbp, Tef) and immune stimulation (e.g., Lyst, Sesn2, Sesn3) were observed following TGFRt15-TGFRs treatment. Results of these RNA-seq studies are shown as heatmaps in FIG. 36.

### Example 13

### Senolytic and Senomorphic Function of TGFRt15-TGFRs In Livers of Young and Aged Mice

To further evaluate whether the TGFPRII component of TGFRt15-TGFRs exhibited senolytic and senomorphic function, young and aged mice were treated with a single-dose of TGFRt15-TGFRs and RNA-seq analysis on livers were performed 10 days after treatment. TGFRt15-TGFRs treatment significantly lowered the expression of Cdkn1a and many circadian clock genes in the liver. A comparison of impacts of TGFRt15-TGFRs and TGFRt15*-TGFRs 120 days after treatment was also performed. RNA-seq analysis on liver from treated mice showed that TGFRt15-TGFRs, but not TGFRt15*-TGFRs, maintained the downregulation of Cdkn1a expression and both treatments continued to upregulate the Tert gene expression compared with PBS treatment as shown in FIG. 36. Interestingly, TGFRt15*-TGFRs treatment significantly increased circadian molecular clock activator genes Arntl and Npas2 compared to TGFRt15-TGFRs-treated or the control group. Since TGFRt15*-TGFRs did not activate or promote proliferation of immune cells, this suggests that direct neutralization of TGF-fβ by the TGFβRII component of TGFRt15-TGFRs may contribute to the senolytic and senomorphic activities of TGFRt15-TGFRs. This also suggests that the IL-15 component of TGFRt15-TGFRs provides long lasting senolytic activity.

Taken together, these Examples indicates that TGFRt15-TGFRs treatment durably reduces genes associated with SNCs and SASP, and enhances the immune-cell activities in naturally aged mice. It also suggests that TGFRt15-TGFRs treatment improves the metabolic function, fibrosis, and circadian rhythms of liver cells of naturally aged mice.

### Example 14

### TGFRt15-TGFRs Treatment is Safe and Tolerated In Mice and Non-Human Primates

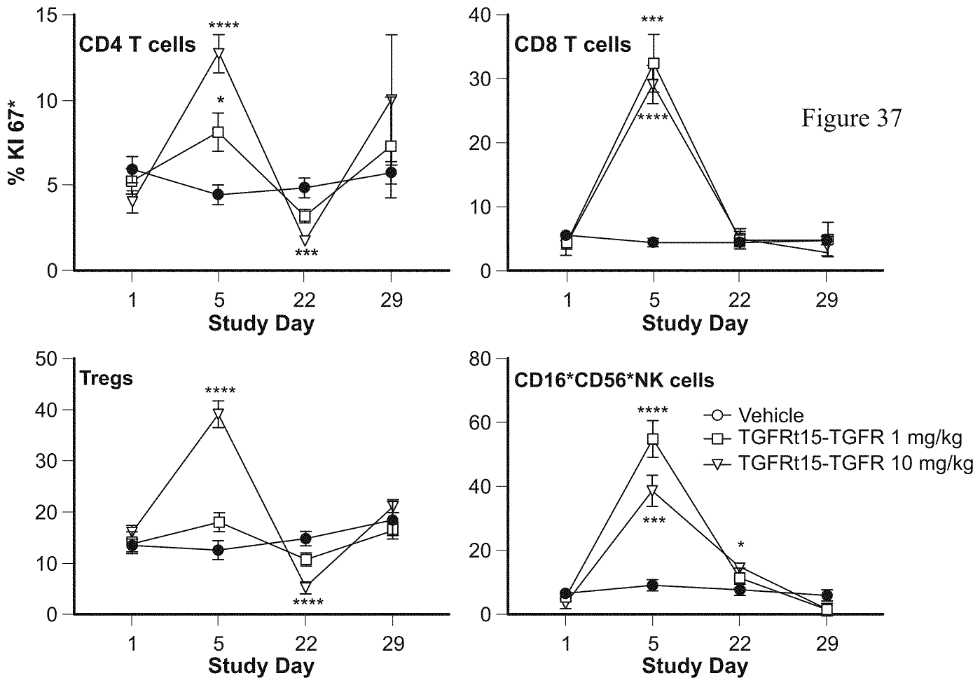

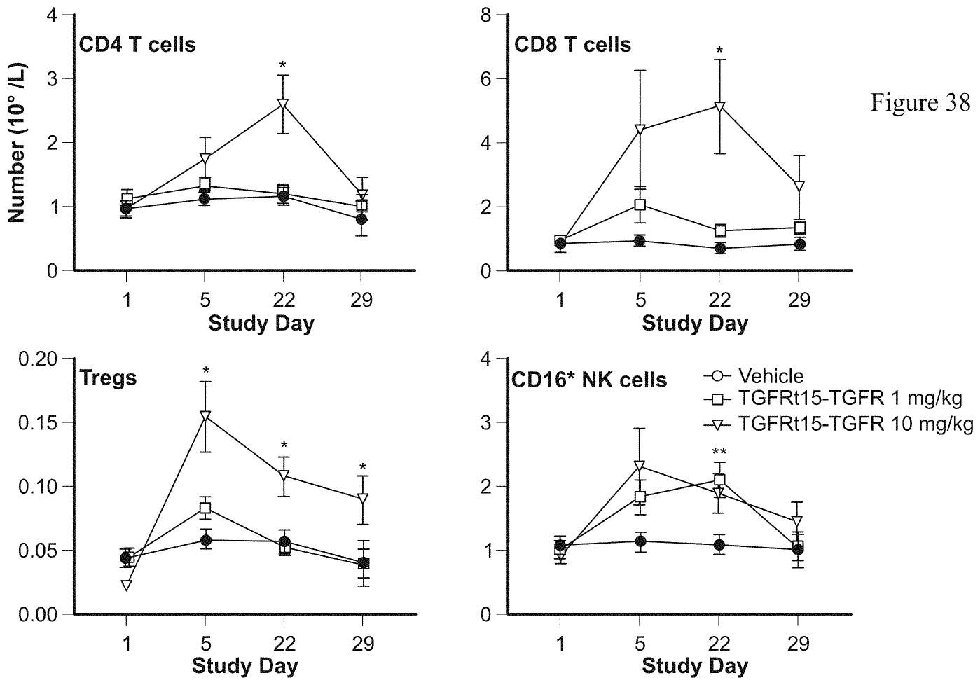

Short-term and long-term toxicity studies of TGFRt15-TGFRs treatment were performed in mice and non-human primates. Subcutaneous administration of TGFRt15-TGFRs at 5 to 100 mg/kg in two doses on days 1 and 15 was well tolerated in a GLP toxicity study in C57BL/6 mice with no observed mortality and no test article related changes in clinical signs or clinical pathology. In a GLP toxicology study in cynomolgus monkeys, subcutaneous administration of TGFRt15-TGFRs at 1 to 10 mg/kg in two doses on days 1 and 15 was also well tolerated. There was no test article related changes in clinical signs, body weight, ophthalmology, ECG, blood pressure, or gross pathology. Dose-dependent increases of MCP-1 and decreases of TGFβ1 and TGFβ2 in the serum were observed. Immunophenotyping indicated that TGFRt15-TGFRs induced dose-dependent increases in the percentage of Ki67+ cells and absolute cell numbers of CD4+, CD8+, Treg and CD16+ NK cells (FIGS. 37 and 38). There was no observed adverse effect of multidose subcutaneous TGFRt15-TGFRs administration in cynomolgus monkeys even at a dose level as high as 10 mg/kg.

Pharmacokinetic analysis showed a half-life of 12 to 21 hours for 1 mg/kg to 10 mg/kg subcutaneously administered TGFRt15-TGFRs in cynomolgus monkeys. The results also confirm that exposure to TGFRt15-TGFRs increased serum levels in a dose-dependent manner with no apparent accumulation of TGFRt15-TGFRs following repeated dosing at 14-day intervals.

The activity and tolerability of TGFRt15-TGFRs was also assessed in naturally aged C57BL/6 mice. 76-week-old mice treated subcutaneously with 3 mg/kg TGFRt15-TGFRs (N=20) or PBS (control; N=20) were observed weekly for changes in body weight and overall survival. In subsequent studies, 90-week-old mice were treated with two subcutaneous 3 mg/kg doses of TGFRt15-TGFRs 45 days apart. Blood was drawn at various time points to assess immune cell subset frequencies. As expected, TGFRt15-TGFRs treatment mediated significant increases in the percentage of CD8+ T cells and NK cells in the blood which returned to baseline 4 weeks post treatment.

TGFRt15-TGFRs treatment was well-tolerated by mice and non-human primates at dose levels significantly higher than the therapeutic dosage (3 mg/kg). There was also no long-term adverse effect of TGFRt15-TGFRs treatment observed on the health span of naturally aged mice.

### Example 15

### TGFRt15-TGFRs Treatment Enhances Immune Cell Populations in db/db Mice

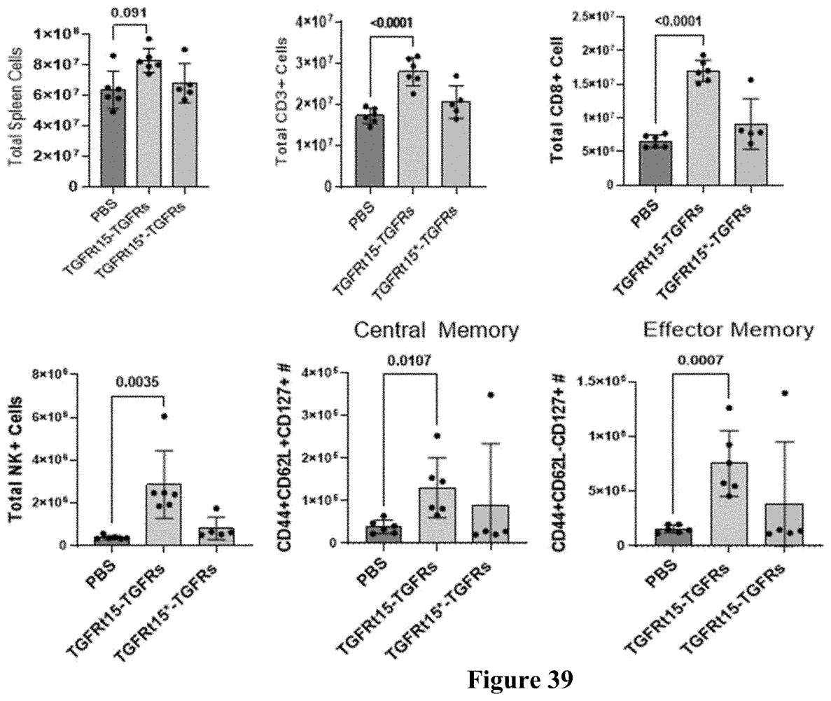

Five-week-old male db/db mice [BKS.Cg-Dock7^{m} +/+ Lepr^{db}/J (Wildtype for Dock7^{m}, Homozygous for Lepr^{db}), strain#000642] from Jackson Lab (Bar Harbor, Me.) were fed with standard chow diet (Irradiated 2018 Teklad global 18% protein rodent diet, Envigo) and received drinking water ad libitum. Mice were divided into three groups as follows: PBS control group (n=6), TGFRt15-TGFRs group (n=6) and TGFRt15*-TGFRs group (n=6). Mice were treated subcutaneously with PBS, TGFRt15-TGFRs (3 mg/kg) and TGFRt15*-TGFRs (3 mg/kg). The mice were euthanized, and spleen was harvested and processed to a single cell suspension. Single cells suspension was prepared in order to evaluate the different subsets of immune cells after treatment with TGFRt15-TGFRs.RBCs were lysed in ACK buffer for 5 minutes at room temperature. The remaining cells were washed in FACS buffer (1X PBS (Hyclone) with 0.5% BSA (EMD Millipore) and 0.001% Sodium Azide (Sigma)). To assess the different types of immune cells in spleen, cells were stained with antibodies specific to cell-surface CD3, CD45, CD8 and NK1.1 (BioLegend) for 30 minutes at RT. After surface staining, cells were washed (1500 RPM for 5 minutes at room temperature) in FACS buffer (1X PBS (Hyclone) with 0.5% BSA (EMD Millipore) and 0.001% Sodium Azide (Sigma)). After two washes, cells were resuspended in fixation buffer and analyzed by Flow Cytometry (Celesta-BD Bioscience). The results in FIG. 39 indicate that treatment with TGFRt15-TGFRs an increase total spleen cells and also increase in the percentages of CD3⁺ CD8⁺, CD3⁻NK1.1⁺, and CD3⁺CD45⁺ immune cells in the spleen subsets, whereas treatment with TGFRt15*-TGFRs had no effect on the percentage of these cell populations. TGFRt15-TGFRs treatment also increase the central and effector memory cells population (FIG. 39). These results suggest that IL-15 activity of TGFRt15-TGFRs plays a role in increasing CD8⁺ T cells and NK cells in the blood of db/db mice and able to proliferate CD3⁺CD8⁺, CD3⁻NK1.1⁺, and CD3⁺CD45⁺immune cells.

### Example 16

### TGFRt15-TGFRs Treatment Enhances Cytotoxic Activity of Splenocytes in db/db Mice after Day 4 Post-Treatment

Five-week-old male db/db mice were purchased from the Jackson Laboratory. Mice were housed in a controlled temperature and controlled light environment. Mice were divided into three groups as follows: Saline control group (n=5), TGFRt15-TGFRs group (n=5) and TGFRt15*-TGFRs group (n=6). Mice were treated subcutaneously with PBS, TGFRt15-TGFRs (3 mg/kg) and TGFRt15*-TGFRs (3 mg/kg). The mice were euthanized, and spleen was harvested and processed to a single cell suspension. Single cells suspension was prepared in order to evaluate cytotoxic activity of splenocytes against Yac-1 cells. Yac-1 cells were labelled with CellTrace Violet and mixed with splenocytes (E:T 20:1) and incubated for 20 hours. The cells were washed and resuspended in complete media containing propidium iodide (PI) solution (Sigma Aldrich, St. Louis, Mo.). The cytotoxicity was assessed by flow cytometry as previously described.

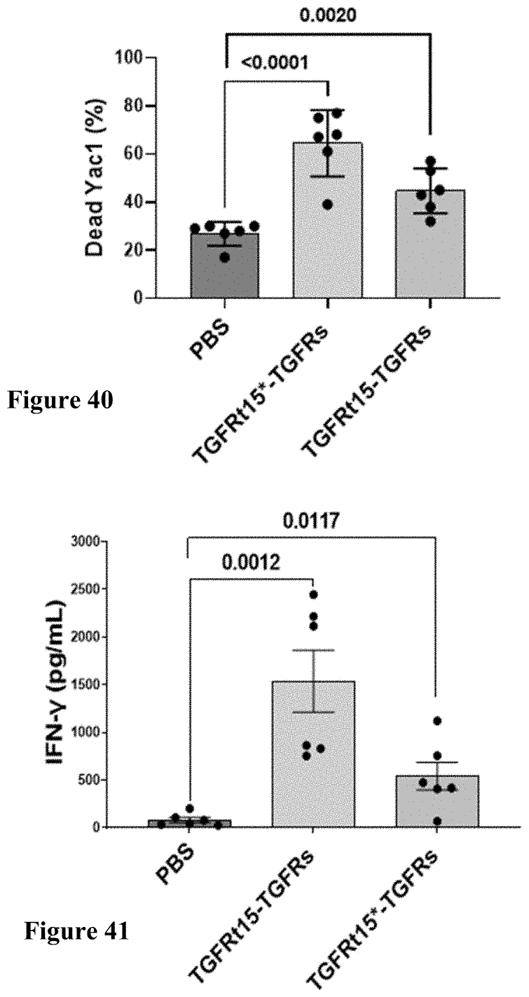

The results in FIG. 40 indicate that treatment with TGFRt15-TGFRs significantly increase the cytotoxic activity of splenocytes compared to TGFRt15*-TGFRs treated splenocytes.

### Example 17

### TGFRt15-TGFRs Treatment Enhances IFN-gamma Production of Splenocytes in db/db Mice After Day 4 Post-Treatment and In Vitro aCD3/CD28 Stimulation Assays

Five-week-old male db/db mice were purchased from the Jackson Laboratory. Mice were housed in a controlled temperature and controlled light environment. Mice were divided into three groups as follows: Saline control group (n =5), TGFRt15-TGFRs group (n =5) and TGFRt15*-TGFRs group (n =6). Mice were treated subcutaneously with PBS, TGFRt15-TGFRs (3 mg/kg) and TGFRt15*-TGFRs (3 mg/kg). The mice were euthanized, and spleen was harvested and processed to a single cell suspension. Single cells suspension were plated at 2×10⁵ cells/well in 96 well U-bottom plate and stimulated with Miltyeni T Cell Activation/Expansion Kit at a 1:1 ratio of beads to cells. Cells were cultured for 4 days, and supernatant was collected to measure TNF-a or IFN-y cytokine released by using

Magpix multiplexing cytokine assay.

The data in FIG. 41 show that both TGFRt15-TGFRs and TGFRt15*-TGFRs enhance interferon-gamma production of splenocytes in db/db mice after day 4 post-treatment and in vitro aCD3/CD28 stimulation assays.

### Example 18

### TGFRt15-TGFRs Treatment Enhances Glycolytic Activity of Splenocytes in db/db Mice After Day 4 Post-Treatment

Five-week-old male db/db mice were purchased from the Jackson Laboratory. Mice were housed in a controlled temperature and controlled light environment. Mice were divided into three groups as follows: Saline control group (n=5), TGFRt15-TGFRs group (n=5) and TGFRt15*-TGFRs group (n =6). Mice were treated subcutaneously with PBS, TGFRt15-TGFRs (3 mg/kg) and TGFRt15*-TGFRs (3 mg/kg). The mice were euthanized at day 4, and spleen was harvested and processed to a single cell suspension. Single cells suspension was prepared in order to measure the glycolytic activity of the splenocytes, the cells were washed and resuspended in seahorse media and resuspended in 4×10⁶ cells/mL. Cells were seeded at 50 μI/well in Cell-Tak-coated Seahorse Bioanalyzer XFe96 culture plates in Seahorse XF RPMI medium, pH 7.4 supplemented with 2 mM L-glutamine for glycolysis stress test. The cells were allowed to attach to the plate for 30 mins at 37° C. Additionally, 130 μl of the assay medium was added to each well of the plate (also the background wells). The plate was incubated in 37° C., non-Co₂ incubator for 1 hr. For glycolysis stress test the calibration plate contained 10x solution of Glucose/oligomycin/2DG prepared in Seahorse assay media and 20 μL of Glucose/oligomycin/2DG were added to each of the ports of the extracellular flux plate that was calibrated overnight. The glycolysis stress test is based on extracellular acidification rate (ECAR) and measures three key parameters of glycolytic function including glycolysis, glycolytic capacity and glycolytic reserve. Complete ECAR analysis consisted of four stages: non glycolytic acidification (without drugs), glycolysis (10 mM glucose), maximal glycolysis induction/glycolytic capacity (2 μM oligomycin), and glycolysis reserve (100 mM 2-DG). At the end of the experiment the data was exported as a Graph Pad Prism file. The XF glycolysis stress test report generator automatically calculated the XF cell glycolysis stress test parameters from the Wave data.

The data was analyzed using the Wave software (Agilent).

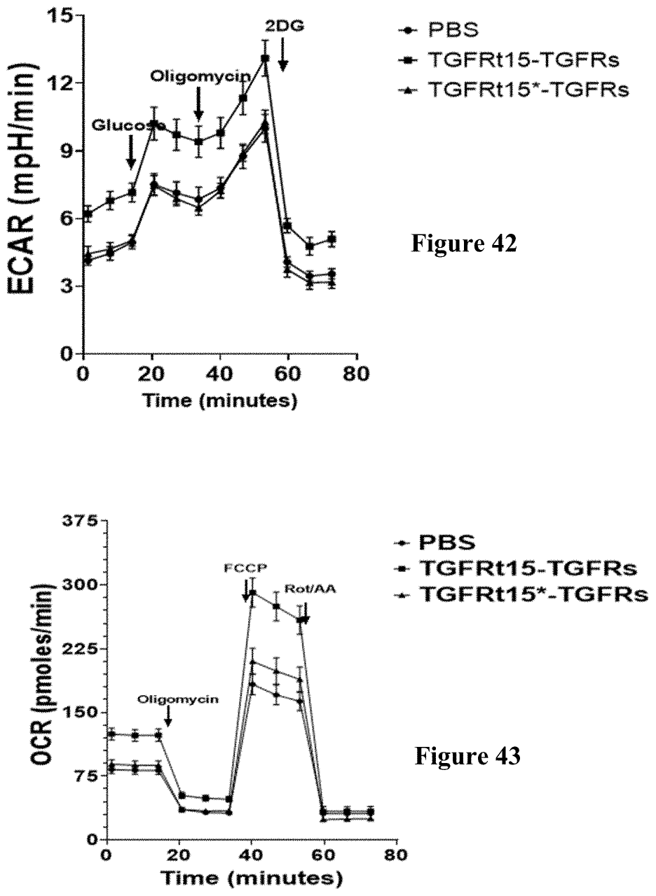

As shown in FIG. 42, the splenocytes isolated from db/db mice at day 4 after TGFRt15-TGFRs therapy showed enhanced basal glycolysis, capacity, and reserve rate, when compared to splenocytes of the saline or TGFRt15*-TGFRs treatment groups.

### Example 19

### TGFRt15-TGFRs Treatment Enhances Mitochondrial Respiration of Splenocytes in db/db Mice After Day 4 Post-Treatment

Five-week-old male db/db mice were purchased from the Jackson Laboratory. Mice were housed in a controlled temperature and controlled light environment. Mice were divided into three groups as follows: Saline control group (n=5), TGFRt15-TGFRs group (n=5) and

TGFRt15*-TGFRs group (n =6). Mice were treated subcutaneously with PBS, TGFRt15-TGFRs (3 mg/kg) and TGFRt15*-TGFRs (3 mg/kg). The mice were euthanized at day 4, and spleen was harvested and processed to a single cell suspension. Single cells suspension was prepared in order to measure the glycolytic activity of the splenocytes, the cells were washed and resuspended in seahorse media and resuspended in 4×10⁶ cells/mL. Cells were seeded at 50 μl/well in Cell-Tak-coated Seahorse Bioanalyzer XFe96 culture plates in Seahorse XF RPMI medium, pH 7.4 supplemented with 2 mM L-glutamine for glycolysis stress test. The cells were allowed to attach to the plate for 30 mins at 37° C. Additionally, 130 μl of the assay medium was added to each well of the plate (also the background wells). The plate was incubated in 37° C., non-CO₂ incubator for 1 hr. For mitochondrial stress test, the Calibration plate contained 10× solution of oligomycin/FCCP/Rotenone prepared in Seahorse assay media and 20 μL of oligomycin, FCCP and Rotenone was added to each of the ports of the extracellular flux plate that was calibrated overnight. Oxygen Consumption Rate (OCR) was measured using an XFe96 Extracellular Flux Analyzer. Complete OCR analysis consisted of four stages: basal respiration (without drugs), ATP-linked respiration/Proton leak (1.5 μM mM Oligomycin), maximal respiration (2 μM FCCP), and spare respiration (0.5 μM Rotenone). At the end of the experiment, the data was exported as a Graph Pad Prism file. The XF mitochondrial stress test report generator automatically calculates the XF mitochondrial stress test parameters from the Wave data that have been exported to Excel. The data was analyzed by using the Wave software (Agilent).

As shown in FIG. 43, the splenocytes isolated from db/db mice at day 4 after TGFRt15-TGFRs therapy showed enhanced basal respiration, mitochondria respiration, capacity, and ATP production, when compared to splenocytes of the saline or TGFRt15*-TGFRs treatment groups.

### Example 20

### TGFRt15-TGFRs Treatment Decreases Plasma TGFI31 and TGFI32 Levels in db/db Mice After Day Post-Treatment

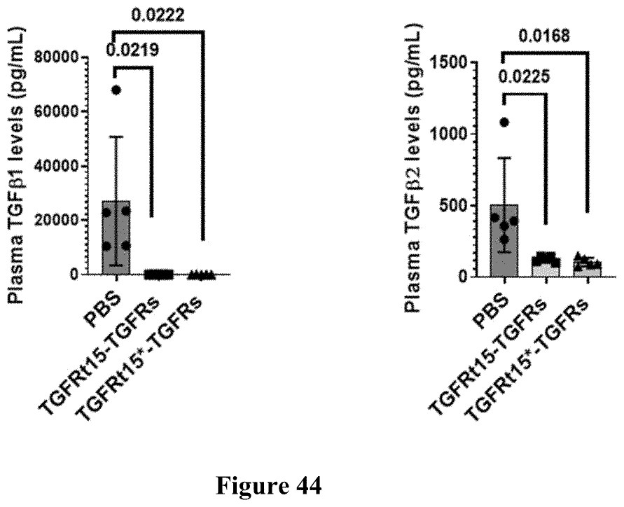

Five-week-old male db/db mice were purchased from the Jackson Laboratory. Mice were housed in a controlled temperature and controlled light environment. Mice were divided into three groups as follows: Saline control group (n=5), TGFRt15-TGFRs group (n=5) and TGFRt15*-TGFRs group (n=6). Mice were treated subcutaneously with PBS, TGFRt15-TGFRs (3 mg/kg) and TGFRt15*-TGFRs (3 mg/kg). Blood was collected from the submandibular vein in tubes containing EDTA and plasma was isolated by centrifugation. The plasma TGF-f3 levels were analyzed by using cytokine array, TGFβ3-plex (TGFβ 1-3) (Eve Technologies, Calgary, AL, Canada). As shown in FIG. 44, plasma TGFβ1 and 2 levels were decrease in db/db mice at day 4 after TGFRt15-TGFRs treatment compared to PBS or TGFRt15*-TGFRs treatment groups.

### Example 21

### Generation of TGFRt15*-TGFRs

A fusion protein complex was generated comprising of TGFR/IL15RαSu and

TGFR/TF/IL-15D8N fusion proteins. The human TGF-b receptor (TGFR), IL-15 alpha receptor sushi domain (IL15RaSu), tissue factor (TF) and IL-15 with D8N mutant (IL15D8N) sequences were obtained from the GenBank website and DNA fragments for these sequences were synthesized by Genewiz. Specifically, a construct was made linking the TGFR sequence to the N-terminus coding region of IL15RaSu and the TGFR sequence to the N-terminus of tissue factor 219 followed by the N-terminus coding region of IL-15D8N.

The nucleic acid sequence of the TGFR/IL15RaSu construct (including signal peptide sequence) is as follows:

(Signal peptide)
ATGAAGTGGGTGACCTTCATCAGCCTGCTGTTCCT
GTTCTCCAGCGCCTACTCC
(Single chain Human TGF-beta
Receptor II homodimer)
ATCCCCCCCCATGTGCAAAAGAGCGTGAACAACGA
TATGATCGTGACCGACAACAACGGCGCCGTGAAGT
TTCCCCAGCTCTGCAAGTTCTGCGATGTCAGGTTC
AGCACCTGCGATAATCAGAAGTCCTGCATGTCCAA
CTGCAGCATCACCTCCATCTGCGAGAAGCCCCAAG
AAGTGTGCGTGGCCGTGTGGCGGAAAAATGACGAG
AACATCACCCTGGAGACCGTGTGTCACGACCCCAA
GCTCCCTTATCACGACTTCATTCTGGAGGACGCTG
CCTCCCCCAAATGCATCATGAAGGAGAAGAAGAAG
CCCGGAGAGACCTTCTTTATGTGTTCCTGTAGCAG
CGACGAGTGTAACGACAACATCATCTTCAGCGAAG
AGTACAACACCAGCAACCCTGATGGAGGTGGCGGA
TCCGGAGGTGGAGGTTCTGGTGGAGGTGGGAGTAT
TCCTCCCCACGTGCAGAAGAGCGTGAATAATGACA
TGATCGTGACCGATAACAATGGCGCCGTGAAATTT
CCCCAGCTGTGCAAATTCTGCGATGTGAGGTTTTC
CACCTGCGACAACCAGAAGTCCTGTATGAGCAACT
GCTCCATCACCTCCATCTGTGAGAAGCCTCAGGAG
GTGTGCGTGGCTGTCTGGCGGAAGAATGACGAGAA
TATCACCCTGGAAACCGTCTGCCACGATCCCAAGC
TGCCCTACCACGATTTCATCCTGGAAGACGCCGCC
AGCCCTAAGTGCATCATGAAAGAGAAAAAGAAGCC
TGGCGAGACCTTTTTCATGTGCTCCTGCAGCAGCG
ACGAATGCAACGACAATATCATCTTTAGCGAGGAA
TACAATACCAGCAACCCCGAC
(Sushi domain of IL 15 receptor
alpha chain)
(SEQ ID NO: 61)
ATTACATGCCCCCCTCCCATGAGCGTGGAGCACGC
CGACATCTGGGTGAAGAGCTATAGCCTCTACAGCC
GGGAGAGGTATATCTGTAACAGCGGCTTCAAGAGG
AAGGCCGGCACCAGCAGCCTCACCGAGTGCGTGCT
GAATAAGGCTACCAACGTGGCTCACTGGACAACAC
CCTCTTTAAAGTGCATCCGG

The nucleic acid sequence of the TGFR/TF/IL15D8N construct (including signal peptide sequence) is as follows:

(Signal peptide)
ATGGGAGTGAAAGTTCTTTTTGCCCTTATTTGTAT
TGCTGTGGCCGAGGCC
(Single chain Human TGF-beta
Receptor II homodimer)
ATCCCACCGCACGTTCAGAAGTCGGTGAATAACGA
CATGATAGTCACTGACAACAACGGTGCAGTCAAGT
TTCCACAACTGTGTAAATTTTGTGATGTGAGATTT
TCCACCTGTGACAACCAGAAATCCTGCATGAGCAA
CTGCAGCATCACCTCCATCTGTGAGAAGCCACAGG
AAGTCTGTGTGGCTGTATGGAGAAAGAATGACGAG
AACATAACACTAGAGACAGTTTGCCATGACCCCAA
GCTCCCCTACCATGACTTTATTCTGGAAGATGCTG
CTTCTCCAAAGTGCATTATGAAGGAAAAAAAAAAG
CCTGGTGAGACTTTCTTCATGTGTTCCTGTAGCTC
TGATGAGTGCAATGACAACATCATCTTCTCAGAAG
AATATAACACCAGCAATCCTGACGGAGGTGGCGGA
TCCGGAGGTGGAGGTTCTGGTGGAGGTGGGAGTAT
TCCTCCCCACGTGCAGAAGAGCGTGAATAATGACA
TGATCGTGACCGATAACAATGGCGCCGTGAAATTT
CCCCAGCTGTGCAAATTCTGCGATGTGAGGTTTTC
CACCTGCGACAACCAGAAGTCCTGTATGAGCAACT
GCTCCATCACCTCCATCTGTGAGAAGCCTCAGGAG
GTGTGCGTGGCTGTCTGGCGGAAGAATGACGAGAA
TATCACCCTGGAAACCGTCTGCCACGATCCCAAGC
TGCCCTACCACGATTTCATCCTGGAAGACGCCGCC
AGCCCTAAGTGCATCATGAAAGAGAAAAAGAAGCC
TGGCGAGACCTTTTTCATGTGCTCCTGCAGCAGCG
ACGAATGCAACGACAATATCATCTTTAGCGAGGAA
TACAATACCAGCAACCCCGAC
(Human Tissue Factor 219)
TCAGGCACTACAAATACTGTGGCAGCATATAATTT
AACTTGGAAATCAACTAATTTCAAGACAATTTTGG
AGTGGGAACCCAAACCCGTCAATCAAGTCTACACT
GTTCAAATAAGCACTAAGTCAGGAGATTGGAAAAG
CAAATGCTTTTACACAACAGACACAGAGTGTGACC
TCACCGACGAGATTGTGAAGGATGTGAAGCAGACG
TACTTGGCACGGGTCTTCTCCTACCCGGCAGGGAA
TGTGGAGAGCACCGGTTCTGCTGGGGAGCCTCTGT
ATGAGAACTCCCCAGAGTTCACACCTTACCTGGAG
ACAAACCTCGGACAGCCAACAATTCAGAGTTTTGA
ACAGGTGGGAACAAAAGTGAATGTGACCGTAGAAG
ATGAACGGACTTTAGTCAGAAGGAACAACACTTTC
CTAAGCCTCCGGGATGTTTTTGGCAAGGACTTAAT
TTATACACTTTATTATTGGAAATCTTCAAGTTCAG
GAAAGAAAACAGCCAAAACAAACACTAATGAGTTT
TTGATTGATGTGGATAAAGGAGAAAACTACTGTTT
CAGTGTTCAAGCAGTGATTCCCTCCCGAACAGTTA
ACCGGAAGAGTACAGACAGCCCGGTAGAGTGTATG
GGCCAGGAGAAAGGGGAATTCAGAGAA
(Human IL-15D8N)
AACTGGGTGAATGTAATAAGTAATTTGAAAAAAAT
TGAAGATCTTATTCAATCTATGCATATTGATGCTA
CTTTATATACGGAAAGTGATGTTCACCCCAGTTGC
AAAGTAACAGCAATGAAGTGCTTTCTCTTGGAGTT
ACAAGTTATTTCACTTGAGTCCGGAGATGCAAGTA
TTCATGATACAGTAGAAAATCTGATCATCCTAGCA
AACAACAGTTTGTCTTCTAATGGGAATGTAACAGA
ATCTGGATGCAAAGAATGTGAGGAACTGGAGGAAA
AAAATATTAAAGAATTTTTGCAGAGTTTTGTACAT
ATTGTCCAAATGTTCATCAACACTTCT

The amino acid sequence of TGFR/IL15RaSu fusion protein (including signal peptide sequence) is as follows:

(Signal peptide)
MKWVTFISLLFLFSSAYS
(Single chain Human TGF-beta
Receptor II homodimer)
IPPHVQKSVNNDMIVTDNNGAVKFPQLCKFCDVRF
STCDNQKSCMSNCSITSICEKPQEVCVAVWRKNDE
NITLETVCHDPKLPYHDFILEDAASPKCIMKEKKK
PGETFFMCSCSSDECNDNIIFSEEYNTSNPDGGGG
SGGGGSGGGGSIPPHVQKSVNNDMIVTDNNGAVKF
PQLCKFCDVRFSTCDNQKSCMSNCSITSICEKPQE
VCVAVWRKNDENITLETVCHDPKLPYHDFILEDAA
SPKCIMKEKKKPGETFFMCSCSSDECNDNIIFSEE
YNTSNPD
(Human IL-15 receptor a sushi domain)
(SEQ ID NO: 8)
ITCPPPMSVEHADIWVKSYSLYSRERYICNSGFKR
KAGTSSLTECVLNKATNVAHWTTPSLKCIR

The amino acid sequence of TGFR/TF/IL15D8N fusion protein (including signal peptide sequence) is as follows:

(Signal peptide)
MGVKVLFALICIAVAEA
(Single chain Human TGF-beta
Receptor II homodimer)
IPPHVQKSVNNDMIVTDNNGAVKFPQLCKFCDVRF
STCDNQKSCMSNCSITSICEKPQEVCVAVWRKNDE
NITLETVCHDPKLPYHDFILEDAASPKCIMKEKKK
PGETFFMCSCSSDECNDNIIFSEEYNTSNPDGGGG
SGGGGSGGGGSIPPHVQKSVNNDMIVTDNNGAVKF
PQLCKFCDVRFSTCDNQKSCMSNCSITSICEKPQE
VCVAVWRKNDENITLETVCHDPKLPYHDFILEDAA
SPKCIMKEKKKPGETFFMCSCSSDECNDNIIFSEE
YNTSNPD
(Tissue factor)
SGTTNTVAAYNLTWKSTNFKTILEWEPKPVNQVYT
VQISTKSGDWKSKCFYTTDTECDLTDEIVKDVKQT
YLARVFSYPAGNVESTGSAGEPLYENSPEFTPYLE
TNLGQPTIQSFEQVGTKVNVTVEDERTLVRRNNTF
LSLRDVFGKDLIYTLYYWKSSSSGKKTAKTNTNEF
LIDVDKGENYCFSVQAVIPSRTVNRKSTDSPVECM
GQEKGEFRE
(IL-15D8N)
(SEQ ID NO: 68)
NWVNVISNLKKIEDLIQSMHIDATLYTESDVHPSC
KVTAMKCFLLELQVISLESGDASIHDTVENLIILA
NNSLSSNGNVTESGCKECEELEEKNIKEFLQSFVH
IVQMFINTS

The TGFR/IL15RaSu and TGFR/TF/IL-15D8N constructs were cloned into a modified retrovirus expression vectors as described previously (Hughes MS, Yu YY, Dudley ME, Zheng Z, Robbins PF, Li Y, et al). The expression vectors were transfected into CHO-K1 cells. Co-expression of the two constructs in CHO-K1 cells allowed for formation and secretion of the soluble TGFR/IL15RαSu-TGFR/TF/IL-15D8N protein complex (referred to as TGFRt15*-TGFRs), which can be purified by anti-TF antibody affinity.

### Example 22

### Protection of TGFRt15-TGFRs from Chemical Induced Liver Damages

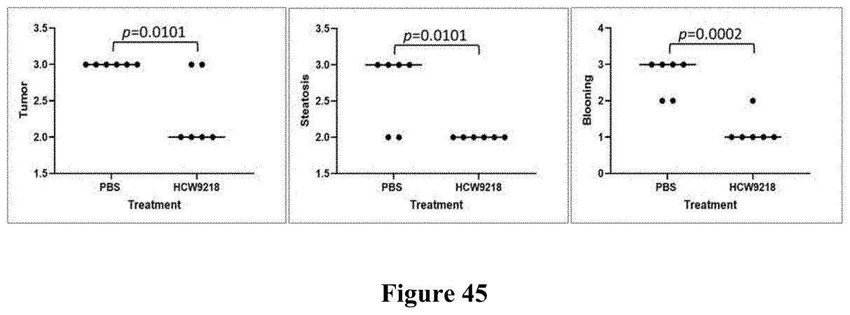

B6C3F1 male mice were purchased from The Jackson Laboratory. The mice were divided into two groups as follows: saline control group (n=6) and TGFRt15-TGFRs group (n=6). All mice (14-day-old) were peritoneally treated with DEN (1 mg/kg, diethylnitrosamine) on study day zero (SDO). CC₄ (0.2 mL/kg, carbon tetrachloride) was peritoneally injected into the mice at 8 weeks of age (SD42) and continued to treat at twice a week for up to 14 additional weeks. TGFRt15-TGFRs was subcutaneously injected (3 mg/kg) on SD43 and SD71. The treated mice were euthanized on SD161 and the livers were harvested and embedded in 4% formalin. The liver sections were stained with hematoxylin and eosin. Tumor, steatosis and hepatocellular ballooning were examined under the light microscope. The severity of liver damage was expressed as mild (1), moderate (2) and extensive (3). Statistical analyses were performed using GraphPad Prism 9 by unpaired t test. For each test, a P value of less than 0.05 was considered statistically significant. As shown in FIG. 45, TGFRt15-TGFRs significantly inhibited liver tumor development and growth, steatosis and hepatocellular ballooning induced by DEN and CCl₄ in B6C3F1 mice.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a liver disease or a metabolic syndrome in a subject, wherein the method comprises administering to the subject a therapeutically effective amount of a multi-chain chimeric polypeptide comprising:
(a) a first chimeric polypeptide comprising:
(i) a first target-binding domain;
(ii) soluble tissue factor domain; and
(iii) a first domain of a pair of affinity domains;
(b) a second chimeric polypeptide comprising:
(i) a second domain of a pair of affinity domains; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and the second target-binding domain binds specifically to a ligand of TGF-βRII.

2. The method of claim 1, wherein the liver disease is selected from the group consisting of: fatty liver disease, hepatic steatosis, acute hepatic porphyria, Alagille syndrome, alcohol-related liver disease, alpha-1 anti-trypsin deficiency, autoimmune hepatitis, benign liver tumors, cholangiocarcinoma, biliary atresia, Budd-Chiari syndrome, cirrhosis, Crigler-Najjar syndrome, galactosemia, Gilbert syndrome, hemochromatosis, hepatic encephalopathy, hepatitis A, hepatitis B, hepatitis C, hepatorenal syndrome, intrahepatic cholestasis of pregnancy (ICP), lysosomal acid lipase deficiency (LAL-D), liver cysts, liver cancer, newborn jaundice, non-alcoholic fatty liver disease, non-alcoholic steatohepatitis, primary biliary cholangitis (PBC), primary sclerosing cholangitis (PSC), progressive familial intrahepatic cholestasis (PFIC), Reye's syndrome, type 1 glycogen storage disease, and Wilson's disease.

3. The method of claim 1, wherein the metabolic syndrome is selected from the group consisting of: coronary heart disease, pulmonary disease, gall bladder disease, dyslipidemia, hypertension, type 2 diabetes, dementia, cancer, gynecological abnormalities including polycystic ovarian syndrome, osteoarthritis, pancreatitis, idiopathic intracranial hypertension, stroke, and cataracts.

4. A method of reducing one or more of the rate of: progression from non-alcoholic fatty liver disease (NAFL) to non-alcoholic steatohepatitis (NASH), progression from NASH to cirrhosis, and progression from cirrhosis to hepatocellular carcinoma, comprising administering to a subject identified or diagnosed as having NAFL, NASH, or cirrhosis, a therapeutically effective amount of a multi-chain chimeric polypeptide comprising:
(a) a first chimeric polypeptide comprising:
(i) a first target-binding domain;
(ii) soluble tissue factor domain; and
(iii) a first domain of a pair of affinity domains;
(b) a second chimeric polypeptide comprising:
(i) a second domain of a pair of affinity domains; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and the second target-binding domain binds specifically to a ligand of TGF-PRII.

5.-7. (canceled)

8. A method of reducing inflammation in a liver of a subject, wherein the method comprises administering to the subject a therapeutically effective amount of a multi-chain chimeric polypeptide comprising:
(a) a first chimeric polypeptide comprising:
(i) a first target-binding domain;
(ii) soluble tissue factor domain; and
(iii) a first domain of a pair of affinity domains;
(b) a second chimeric polypeptide comprising:
(i) a second domain of a pair of affinity domains; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and the second target-binding domain binds specifically to a ligand of TGF-βRII.

9. A method of decreasing gluconeogenesis in a liver of a subject, wherein the method comprises administering to the subject a therapeutically effective amount of a multi-chain chimeric polypeptide comprising:
(a) a first chimeric polypeptide comprising:
(i) a first target-binding domain;
(ii) soluble tissue factor domain; and
(iii) a first domain of a pair of affinity domains;
(b) a second chimeric polypeptide comprising:
(i) a second domain of a pair of affinity domains; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and the second target-binding domain binds specifically to a ligand of TGF-βRII.

10. A method of decreasing lipogenesis in a liver of a subject, wherein the method comprises administering to the subject a therapeutically effective amount of a multi-chain chimeric polypeptide comprising:
(a) a first chimeric polypeptide comprising:
(i) a first target-binding domain;
(ii) soluble tissue factor domain; and
(iii) a first domain of a pair of affinity domains;
(b) a second chimeric polypeptide comprising:
(i) a second domain of a pair of affinity domains; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and the second target-binding domain binds specifically to a ligand of TGF-βRII.

11. A method of decreasing hepatocytic senescence in a liver of a subject, wherein the method comprises administering to the subject a therapeutically effective amount of a multi-chain chimeric polypeptide comprising:
(a) a first chimeric polypeptide comprising:
(i) a first target-binding domain;
(ii) soluble tissue factor domain; and
(iii) a first domain of a pair of affinity domains;
(b) a second chimeric polypeptide comprising:
(i) a second domain of a pair of affinity domains; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and the second target-binding domain binds specifically to a ligand of TGF-βRII.

12. A method of rebalancing metabolic function in a liver of a subject, wherein the method comprises administering to the subject a therapeutically effective amount of a multi-chain chimeric polypeptide comprising:
(a) a first chimeric polypeptide comprising:
(i) a first target-binding domain;
(ii) soluble tissue factor domain; and
(iii) a first domain of a pair of affinity domains;
(b) a second chimeric polypeptide comprising:
(i) a second domain of a pair of affinity domains; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and the second target-binding domain binds specifically to a ligand of TGF-βRII.

13. A method of modulating expression of one or more genes in Tables 1-4 in a liver of a subject, wherein the method comprises administering to the subject a therapeutically effective amount of a multi-chain chimeric polypeptide comprising:
(a) a first chimeric polypeptide comprising:
(i) a first target-binding domain;
(ii) soluble tissue factor domain; and
(iii) a first domain of a pair of affinity domains;
(b) a second chimeric polypeptide comprising:
(i) a second domain of a pair of affinity domains; and
(ii) a second target-binding domain,
wherein:
the first chimeric polypeptide and the second chimeric polypeptide associate through the binding of the first domain and the second domain of the pair of affinity domains; and
the first target-binding domain binds specifically to a ligand of TGF-β receptor II (TGF-βRII) and the second target-binding domain binds specifically to a ligand of TGF-βRII.

14.-17. (canceled)

18. The method of claim 8, wherein the subject has been previously identified or diagnosed as having a liver disease or a metabolic syndrome.

19.-22. (canceled)

23. The method of claim 1, wherein the first target-binding domain and the soluble tissue factor domain directly abut each other in the first chimeric polypeptide.

24. The method of claim 1, wherein the first chimeric polypeptide further comprises a linker sequence between the first target-binding domain and the soluble tissue factor domain in the first chimeric polypeptide.

25. The method of claim 1, wherein the soluble tissue factor domain and the first domain of the pair of affinity domains directly abut each other in the first chimeric polypeptide.

26. The method of claim 1, wherein the first chimeric polypeptide further comprises a linker sequence between the soluble tissue factor domain and the first domain of the pair of affinity domains in the first chimeric polypeptide.

27. The method of claim 1, wherein the second domain of the pair of affinity domains and the second target-binding domain directly abut each other in the second chimeric polypeptide.

28. The method of claim 1, wherein second chimeric polypeptide further comprises a linker sequence between the second domain of the pair of affinity domains and the second target-binding domain in the second chimeric polypeptide.

29. The method of claim 1, wherein one or both of the first target-binding domain and the second target-binding domain is an antigen-binding domain.

30. The method of claim 1, wherein one or both of the first target-binding domain and the second target-binding domain is a soluble interleukin or cytokine receptor.

31.-32. (canceled)

33. The method of claim 1, wherein the soluble tissue factor domain is a soluble human tissue factor domain.

34. The method of claim 33, wherein the soluble human tissue factor domain comprises a sequence that is at least 80% identical to SEQ ID NO: 1.

35. The method of claim 1, wherein the pair of affinity domains is a sushi domain from an alpha chain of human IL-15 receptor (IL-15Rα) and a soluble IL-15.

36. The method of claim 1, wherein the first target-binding domain comprises a soluble TGF-βRII.

37. The method of claim 36, wherein the first target-binding domain comprises a first sequence that is at least 80% identical to SEQ ID NO: 2 and a second sequence that is at least 80% identical to SEQ ID NO: 2, wherein the first and second sequence are separated by a linker.

38.-39. (canceled)

40. The method of claim 37, wherein the linker comprises a sequence of SEQ ID NO: 3.

41. The method of claim 36, wherein the first target-binding domain comprises a sequence that is at least 80% identical to SEQ ID NO: 4.

42.-43. (canceled)

44. The method of claim 36, wherein the first chimeric polypeptide comprises a sequence that is at least 80% identical to SEQ ID NO: 6.

45.-47. (canceled)

48. The method of claim 1, wherein the second target-binding domain comprises a soluble TGF-βRII.

49. The method of claim 48, wherein the second target-binding domain comprises a first sequence that is at least 80% identical to SEQ ID NO: 2 and a second sequence that is at least 80% identical to SEQ ID NO: 2, wherein the first and second sequence are separated by a linker.

50.-51. (canceled)

52. The method of claim 49, wherein the linker comprises a sequence of SEQ ID NO: 3.

53. The method of claim 48, wherein the second target-binding domain comprises a sequence that is at least 80% identical to SEQ ID NO: 4.

54.-55. (canceled)

56. The method of claim 48, wherein the second chimeric polypeptide comprises a sequence that is at least 80% identical to SEQ ID NO: 5.

57. The method of claim 56, wherein the first chimeric polypeptide comprises a sequence that is at least 80% identical to SEQ ID NO: 6.

58.-61. (canceled)
